SEQUENCE LISTING (54 sequences)

SEQ ID NO: 1 - DNA, 645 bp, synthetic construct
  source 1..645: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 2109 bp, synthetic construct
  source 1..2109: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 4 - protein, 702 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 5 - DNA, 660 bp, synthetic construct
  source 1..660: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 219 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 2121 bp, synthetic construct
  source 1..2121: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcacaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 8 - protein, 706 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 9 - DNA, 1422 bp, synthetic construct
  source 1..1422: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggttgtgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtggcggcggaagtggcggtggaggatccggcggtggtggatcacaggtgcagctgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 10 - protein, 473 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 11 - DNA, 1347 bp, synthetic construct
  source 1..1347: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagtgcctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 12 - protein, 448 aa, synthetic construct
QVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - DNA, 645 bp, synthetic construct; identical to SEQ ID NO: 1
  source 1..645: mol_type = other DNA

SEQ ID NO: 14 - protein, 214 aa, synthetic construct; identical to SEQ ID NO: 2

SEQ ID NO: 15 - DNA, 2109 bp, synthetic construct
  source 1..2109: mol_type = other DNA
caggtgcagctgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggcggtggaggatccggcggtggtggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 16 - protein, 702 aa, synthetic construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - DNA, 1407 bp, synthetic construct
  source 1..1407: mol_type = other DNA
caggtgcagctgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggcggtggaggatccggcggtggtggatcagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggttgtgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 18 - protein, 468 aa, synthetic construct
QVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1347 bp, synthetic construct; identical to SEQ ID NO: 11
  source 1..1347: mol_type = other DNA

SEQ ID NO: 20 - protein, 448 aa, synthetic construct; identical to SEQ ID NO: 12

SEQ ID NO: 21 - DNA, 1437 bp, synthetic construct
  source 1..1437: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggttgcggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtggcggcggaagtggcggtggaggatccggcggtggtggatcacaggtgcagctgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagcggtggaggcggttcaggcggaggtggttccggcggtggcggctcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctataa

SEQ ID NO: 22 - protein, 478 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGCGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 23 - DNA, 1359 bp, synthetic construct
  source 1..1359: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagtgtctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 24 - protein, 452 aa, synthetic construct
QVQLQQSGAEVKKPGSSVKVSCKASGYTFTNYYIYWVRQAPGQCLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 11 aa, synthetic construct
RASQSIGTNIH

SEQ ID NO: 26 - protein, 7 aa, synthetic construct
YASESIS

SEQ ID NO: 27 - protein, 9 aa, synthetic construct
QQNNNWPTT

SEQ ID NO: 28 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 29 - protein, 5 aa, synthetic construct
NYGVH

SEQ ID NO: 30 - protein, 16 aa, synthetic construct
VIWSGGNTDYNTPFTS

SEQ ID NO: 31 - protein, 11 aa, synthetic construct
ALTYYDYEFAY

SEQ ID NO: 32 - protein, 5 aa, synthetic construct
SYWMS

SEQ ID NO: 33 - protein, 17 aa, synthetic construct
NINRDGSASYYVDSVKG

SEQ ID NO: 34 - protein, 9 aa, synthetic construct
DRGVGYFDL

SEQ ID NO: 35 - protein, 14 aa, synthetic construct
TGTSSDVGGYNFVS

SEQ ID NO: 36 - protein, 7 aa, synthetic construct
DVSDRPS

SEQ ID NO: 37 - protein, 11 aa, synthetic construct
SSYGSSSTHVI

SEQ ID NO: 38 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 39 - protein, 118 aa, synthetic construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 40 - protein, 111 aa, synthetic construct
QSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVL

SEQ ID NO: 41 - protein, 16 aa, synthetic construct
RSSQNIVHSNGNTYLD

SEQ ID NO: 42 - protein, 7 aa, synthetic construct
KVSNRFS

SEQ ID NO: 43 - protein, 9 aa, synthetic construct
FQYSHVPWT

SEQ ID NO: 44 - protein, 112 aa, synthetic construct [sequence omitted]

SEQ ID NO: 45 - protein, 5 aa, synthetic construct
NYYIY

SEQ ID NO: 46 - protein, 17 aa, synthetic construct
GINPTSGGSNFNEKFKT

SEQ ID NO: 47 - protein, 14 aa, synthetic construct
QGLWFDSDGRGFDF

SEQ ID NO: 48 - protein, 123 aa, synthetic construct [sequence omitted]

SEQ ID NO: 49 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELK

SEQ ID NO: 50 - protein, 118 aa, synthetic construct
QVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 51 - protein, 111 aa, synthetic construct; identical to SEQ ID NO: 40

SEQ ID NO: 52 - protein, 119 aa, synthetic construct
QVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSA

SEQ ID NO: 53 - protein, 112 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGCGTKLQIT

SEQ ID NO: 54 - protein, 123 aa, synthetic construct
QVQLQQSGAEVKKPGSSVKVSCKASGYTFTNYYIYWVRQAPGQCLEWIGGINPTSGGSNFNEKFKTRVTITADESSTTAYMELSSLRSEDTAFYFCTRQGLWFDSDGRGFDFWGQGTTVTVSS

(19) United States
(12) Patent Application Publication
(10) Pub. No.: US 2026/0234288 A1
(43) Pub. Date: Aug. 13, 2026

(54) BISPECIFIC ANTIBODY-CAMPTOTHECIN DRUG CONJUGATE AND PHARMACEUTICAL USE THEREOF

(71) Applicant: SystImmune, Inc., Redmond, WA (US)

(72) Inventors: Yi ZHU, Chengdu (CN); Weili WAN, Chengdu (CN); Tianzi YU, Chengdu (CN); Guili ZHU, Chengdu (CN); Yiying ZHANG, Chengdu (CN); Shi ZHUO, Chengdu (CN); Yong ZHANG, Chengdu (CN); Gangrui LI, Chengdu (CN)

(21) Appl. No.: 19/431,550

(22) Filed: Dec. 23, 2025

Related U.S. Application Data
(62) Division of application No. 18/957,324, filed on Nov. 22, 2024, now Pat. No. 12,577,325.
Application No. 18/957,324 is a continuation of application No. 18/710,044, filed on Nov. 15, 2022, now abandoned.

(30) Foreign Application Priority Data
Nov. 15, 2021 (CN) 202111351599.X

(51) Int. Cl. C07K 16/32; A61K 47/68; A61P 35/00; C07K 16/28

(52) U.S. Cl. CPC C07K 16/32; A61K 47/68037; A61K 47/6851; A61P 35/00; C07K 16/2863; C07K 2317/24; C07K 2317/31

(57) ABSTRACT

A bispecific antibody simultaneously targeting two different epitopes or targets is coupled with a camptothecin drug to form a bispecific antibody-toxin conjugate that is stable in treatment and excellent in uniformity, and a drug-to-antibody ratio (DAR) thereof is 6.0-8.0. The antibody-toxin conjugate has a structure as represented by general formula (I), wherein Ab represents the bispecific antibody simultaneously targeting two different epitopes or targets, which is coupled with a linker-camptothecin drug. In addition, the present invention further relates to a preparation and purification method for the antibody-toxin conjugate, and an application thereof in tumor treatment. Furthermore, the present invention further relates to a linker-drug compound capable of being coupled with Ab to form the antibody-toxin conjugate.

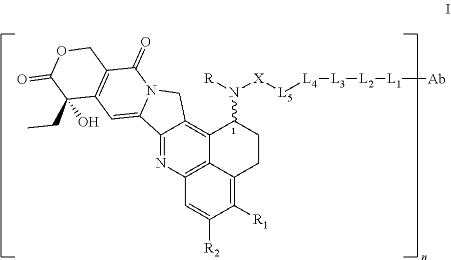

### CROSS-REFERENCE TO RELATED APPLICATIONS

This application is a divisional of U.S. patent application Ser. No. 18/957,324, filed Nov. 22, 2024, now U.S. Pat. No. 12,577,325, which is a continuation application of U.S. patent application Ser. No. 18/710,044, filed May 14, 2024, now abandoned, which is a U.S. National Stage Application under 35 U.S.C. § 371 of International Patent Application No. PCT/CN2022/132027, filed Nov. 15, 2022, which claims priority to CN 202111351599.X, filed Nov. 15, 2021, each of which is incorporated by reference herein in its entirety.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14247-888-999_SEQLISTING.xml”, was created on Dec. 19, 2025, and is 71,054 bytes in size.

### TECHNICAL FIELD

The present invention relates to the field of biopharmaceuticals, and specifically to an antibody-drug conjugate formed by a bispecific antibody and a camptothecin drug, and to a method of preparation and application of the antibody-drug conjugate. The present invention also relates to linker-drug compounds that can be coupled with Ab to form an antibody-toxin conjugate.

### TECHNICAL BACKGROUND

Epidermal Growth Factor Receptor (EGFR) and human epidermal growth factor receptor 3 (also known as HER3 and ErbB3) are receptor protein tyrosine kinases and belong to the epidermal growth factor receptor (EGFR) subfamily of receptor protein tyrosine kinases, which includes EGFR (ErbB-1), HER2/c-neu (ErbB-2), HER3 (ErbB-3) and HER4 (ErbB-4).

The epidermal growth factor receptor is a glycoprotein with a molecular weight of 170 kDa that crosses cell membranes and is activated by binding to ligands. Upon activation, EGFR is converted from a monomer to an auto-dimer or forms a heterodimer with other HER family members. Dimer formation activates intracellular kinase pathways that direct the phosphorylation of downstream pathways, including the MAPK, Akt, and JNK pathways, to induce cell proliferation. Studies have shown that high expression of EGFR is associated with tumor cell proliferation, angiogenesis, tumor invasion, etc. EGFR-related signaling pathways play a very important role in the maintenance and growth of epidermal tissues. Especially in breast cancer, malignant glioma and lung cancer, EGFR can promote tumorigenesis. In lung cancer tissues, the EGFR signaling pathway shows a stimulated state, and there is a positive correlation between the expression level of EGFR and the stage of cancer development. Meanwhile, more and more studies are using EGFR as a biomarker of tumor drug resistance due to the finding of secondary mutations of EGFR under drug stress.

Epidermal growth factor receptor 3 (HER3 or ErbB3) also has the structure of a typical epidermal growth factor receptor, but HER3 lacks the structural domain of the intracellular protein tyrosine kinase and thus cannot autophosphorylate. HER3 can bind to ligand proteins and promote their heterodimerization with other human epidermal growth factor receptor family members to activate receptor-mediated signaling pathways, which not only acts as a signal diversification means, but also plays a role of signal amplification and accelerates tumor progression. Heregulins (glial cell growth factor, neu differentiation factor) can activate intracellular kinase-dependent multistep signaling pathway responses upon binding to the transmembrane receptors HER3 and HER4. Down-regulation of this signaling pathway often leads to Alzheimer's disease, heart failure, atherosclerosis, and cancer, etc. Up-regulation of HER3 expression level can promote tumorigenesis and growth by interacting with receptor tyrosine kinases (RTKs). Also, since HER3 is a heterodimeric molecular chaperone for other EGFR family members, it has the potential to modulate EGFR/HER2 signaling pathway-mediated drug resistance in cancer cells. Studies have also shown that HER3 defeats cancer therapy by activating the PI3K/AKT, MAPK/ERK and JAK/STAT signaling pathways.

Overexpression/dysregulation of EGFR and HER3 has been closely associated with the development of a variety of tumors. EGFR and HER3 have been shown to drive tumor progression in solid tumors such as breast, lung, gastric, and pancreatic cancers. Several studies have shown that high expression of HER3 is associated with clinical failure of EGFR antibodies and inhibitors. Several combination drug therapies targeting EGFR and HER3 are already underway in clinical settings.

Monoclonal antibodies have been widely used in anti-tumor therapy in recent years, but their efficacy leaves much to be desired. A large number of tumor patients have poor clinical responses, and some patients with clinical responses develop drug resistance after a sustained period of monoclonal antibody therapy, leading to tumor recurrence. A bispecific monoclonal antibody is a monoclonal antibody molecule with two different antigen recognition sequences, which can bind protein molecules of two antigenic epitopes, and thus can achieve, for example, a variety of new and unique anti-tumor mechanisms, such as mediating the killing of tumor cells by immune cells, mediating the killing of tumor cells by toxic small molecules, or blocking the signaling pathways that promote the growth of tumors. The development of bispecific monoclonal antibodies is mainly in consideration of the fact that multiple mediators participate in the pathogenesis of tumors through specific or overlapping mechanisms, and if multiple targets are blocked at the same time, this will produce better therapeutic effects than inhibition of a single target, and at the same time, the action of multiple targets greatly reduces the probability of developing drug resistance. Currently, two bispecific antibodies, Catumaxomab and Blinatumomab, have been approved for marketing in the U.S., and more than fifty bispecific antibody molecules are in clinical trials.

Antibody-Drug Conjugates (ADCs) are molecules with specifically targeted killing effects, obtained by attaching small molecule toxins with cell-killing effects to antibodies, and are mainly used in the treatment of tumors and other diseases. Antibody-drug coupled drugs use antibodies that can specifically bind to proteins on the surface of tumor cells, and therefore have tumor specificity and potential unachievable by conventional drugs. Currently, 12 ADC drugs have received marketing approval worldwide and hundreds of programs are in clinical trials. However, most ADC programs currently on the market or in clinical settings are directed at a single target and cannot achieve the synergistic benefits of dual-target therapy.

### SUMMARY OF THE INVENTION

The inventors, based on a comprehensive understanding of the ADC class of drugs, disclose a bispecific antibody-drug conjugate and a method of preparing the same, a pharmaceutical composition comprising said conjugate and a use of said conjugate or pharmaceutical composition. The present invention also relates to linker-drug compounds that can be coupled to a bispecific antibody to form an antibody-toxin conjugate.

A first aspect of the present invention discloses a ligand-camptothecin derivative conjugate as shown in general formula I or a pharmaceutically acceptable salt or solvate thereof,

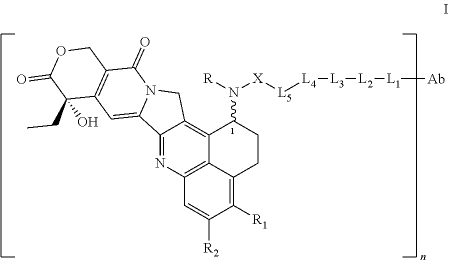

wherein:
Ab is a bispecific antibody or antigen-binding fragment thereof that simultaneously targets two different epitopes or targets;
L₁ is selected without limitation from the group:

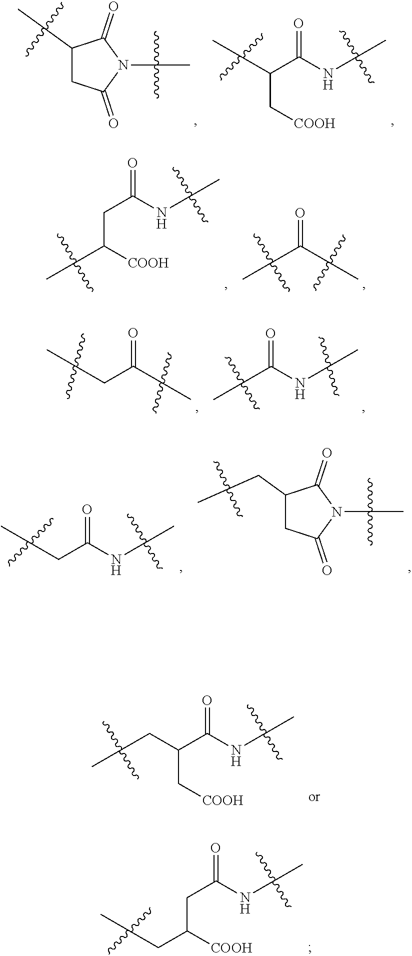

preferably L₁ is

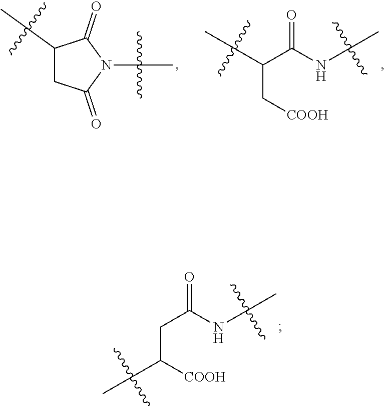 [SMILES: *C1CC(=O)N(*)C1=O ; *NC(=O)C(*)CC(=O)O ; *NC(=O)CC(*)C(=O)O]

preferably L₁ is

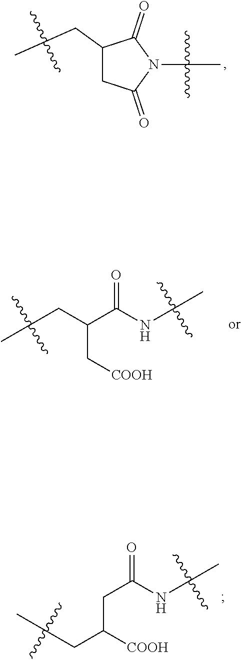 [SMILES: *CC1CC(=O)N(*)C1=O ; *CC(CC(=O)O)C(=O)N* ; *CC(CC(=O)N*)C(=O)O]

L₂ has the structure shown in formula A below,

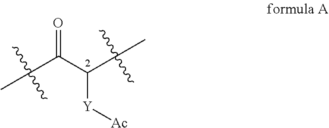

wherein Y is a scaffold selected from C1-C6 alkyl, substituted C1-C6 alkyl, or C3-C8 cycloalkyl; preferably Y is C1-C6 alkyl; Ac is a hydrophilic structural unit; and the carbon No. 2 attached to Y has absolute chirality in the R configuration or the S configuration;
L₃ is present or absent, and when present, L₃ is selected from PEG hydrophilic units:

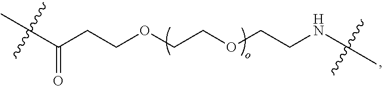

o is selected from an integer in the range of 1-10 (e.g., 1, 2, 3, 4, 5, 6, 7, 8, 9, or 10), preferably an integer in the range of 2-8;
L₄ is an enzymatic cutting unit;
L₅ is a linking unit;
in formula I, the No. 1 chiral carbon atom attached to N has absolute chirality in the R configuration or the S configuration;
R is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, C6-C10 aryl, substituted C6-C10 aryl, 5-10-membered heteroaryl, substituted 5-10-membered heteroaryl;
preferably R is selected from a hydrogen atom or a C1-C6 alkyl group;
R₁ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7-membered heterocyclyl, substituted 3-7-membered heterocyclyl, C6-C10 aryl, substituted C6-C10 aryl, 5-10-membered-heteroaryl, or substituted 5-10-membered heteroaryl;
preferably R₁ is selected from a hydrogen atom or a C1-C6 alkyl group;
more preferably R₁ is selected from C1-C6 alkyl;
R₂ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7-membered heterocyclyl, substituted 3-7-membered heterocyclyl, C6-C10 aryl, substituted C6-C10 aryl, 5-10-membered heteroaryl, substituted 5-10-membered heteroaryl;
preferably R₂ is selected from a hydrogen atom, a halogen or a C1-C6 alkyl group;
more preferably R₂ is selected from halogen;
X is selected from —C(O)—CRₐR_{b}—(CR₃R₄)ₘ—O—, —C(O)—CRₐR_{b}—(CR₃R₄)ₘ—NH— or —C(O)—CRₐR_{b}—(CR₃R₄)ₘ—S—;
preferably X is selected from —C(O)—CRₐR_{b}—(CR₃R₄)ₘ—O—;
Rₐ and R_{b} are each independently selected from a hydrogen atom, a deuterium atom, a halogen, a C1-C6 alkyl, a deuterated C1-C6 alkyl, a halogenated C1-C6 alkyl, a C3-C8 cycloalkyl, a C3-C8 cycloalkyl C1-C6 alkyl, a C6-C10 aryl C1-C6 alkyl, a C1-C6 alkoxy C1-C6 alkyl, a 3-7-membered heterocyclic group, a substituted 3-7-membered heterocyclic group, a C6-C10 aryl, substituted C6-C10 aryl, 5-10-membered heteroaryl, substituted 5-10-membered heteroaryl;
preferably, Rₐ and R_{b} are each independently selected from a hydrogen atom, a C1-C6 alkyl, a halo-C1-C6 alkyl, a C3-C8 cycloalkyl C1-C6 alkyl or a C6-C10 aryl C1-C6 alkyl;
alternatively, Rₐ, R_{b} and the carbon atoms to which they are attached constitute a C3-C8 cycloalkyl group, a C3-C8 cycloalkyl C1-C6 alkyl group, a 3-7-membered heterocyclyl group, a substituted 3-7-membered heterocyclyl group; preferably Rₐ, R_{b} and the carbon atoms to which they are attached constitute a C3-C8 cycloalkyl group;
R₃, R₄ are identical or different and are each independently a hydrogen atom, deuterium atom, halogen, C1-C6 alkyl, halogenated C1-C6 alkyl, deuterated C1-C6 alkyl, C1-C6 alkoxy, hydroxyl, amino, cyano, nitro, hydroxy C1-C6 alkyl, C3-C8 cycloalkyl, 3-7-membered heterocyclyl, or substituted 3-7-membered heterocyclyl;
preferably, R₃, R₄ are each independently a hydrogen atom or C1-C6 alkyl group;
alternatively, R₃, R₄ and the carbon atoms attached thereto constitute a C3-C8 cycloalkyl group, a C3-C8 cycloalkyl C1-C6 alkyl group, a 3-7-membered heterocyclic group, or a substituted 3-7-membered heterocyclic group;
m is selected from integers 0-4 (e.g., 0, 1, 2, 3, or 4), preferably 0, 1; n is selected from integers 1-10 (e.g., 1, 2, 3, 4, 5, 6, 7, 8, 9, or 10).

In some embodiments of the first aspect of the present invention, ligand-camptothecin derivative conjugates as shown in general formula I or pharmaceutically acceptable salts or solvates thereof are disclosed, characterized in that Ab is a bispecific antibody or antigen-binding fragment thereof that simultaneously targets two different epitopes or targets, preferably a bispecific antibody or antigen-binding fragment thereof that simultaneously targets EGFR and HER3.

In some embodiments of the first aspect of the present invention, the disclosure of a ligand-camptothecin derivative conjugate as shown in general formula I, or a pharmaceutically acceptable salt or solvate thereof, is characterized in that the Ab antibody comprises: an IgG1 heavy chain, a κ light chain, and a single-chain Fv (scFv) structural domain; wherein said single-chain Fv (scFv) structural domain forms a construct with the IgG1 heavy chain or the κ light chain; wherein said IgG1 heavy chain and κ light chain form an IgG portion with binding specificity for EGFR; said scFv structural domain has binding specificity for HER3, and the scFv structural domain is connected by a linker (e.g., having an amino acid sequence of (gly-gly-gly-gly-ser)n, wherein n is an integer of at least 1, preferably n is an integer of 1 to 10) to the C-terminus or N-terminus of said IgG1 heavy chain or C-terminus or N-terminus of said κ light chain; and wherein the single-chain Fv structural domain has a structural order of N-terminus-heavy chain variable region-joint-light chain variable region-C-terminus or N-terminus-light chain variable region-joint-heavy chain variable region-C-terminus (e.g., said joint consists of an amino acid sequence of (gly-gly-gly-gly-ser)m, wherein m is an integer of at least 3, preferably m is 3, 4, 5, or 6).

In some embodiments of the first aspect of the present invention, the disclosure of a ligand-camptothecin derivative conjugate as shown in general formula I, or a pharmaceutically acceptable salt or solvate thereof, is characterized in that the t light chain of the Ab antibody comprises CDRs as shown in SEQ ID NO: 25, SEQ ID NO: 26, and SEQ ID NO: 27, and the IgG1 heavy chain comprises CDRs as shown in SEQ ID NO: 29, SEQ ID NO: 30, and SEQ ID NO: 31, and the single-chain Fv (scFv) structural domain comprises the heavy chain variable region CDRs as shown in SEQ ID NO: 32, SEQ ID NO: 33, and SEQ ID NO: 34, and light chain variable region CDRs as shown in SEQ ID NO: 35, SEQ ID NO: 36, and SEQ ID NO: 37.

In some embodiments of the first aspect of the present invention, the ligand-camptothecin derivative conjugate shown in general formula I, or a pharmaceutically acceptable salt or solvate thereof, is disclosed, characterized in that the light chain of the Ab antibody comprises a variable region as shown in SEQ ID NO: 28, the IgG1 heavy chain comprises a variable region as shown in SEQ ID NO: 38, and the single chain Fv (scFv) structural domain comprises a heavy chain variable region as shown in SEQ ID NO: 39 and a light chain variable region as shown in SEQ ID NO: 40 In some embodiments of the first aspect of the present invention, a ligand-camptothecin derivative conjugate shown in general formula I, or a pharmaceutically acceptable salt or solvate thereof, is disclosed, characterized in that the light chain amino acid sequence of the Ab antibody is SEQ ID NO: 2, and the amino acid sequence of the construct of the antibody heavy chain and the single-chain Fv (scFv) structural domain is SEQ ID NO: 4.

In some embodiments of the first aspect of the present invention, the ligand-camptothecin derivative conjugate shown in general formula I, or a pharmaceutically acceptable salt or solvate thereof, is disclosed, characterized in that the light chain nucleic acid coding sequence of the Ab antibody is SEQ ID NO: 1, and the nucleic acid coding sequence of the construct of the antibody heavy chain and the single-chain Fv (scFv) structural domain is SEQ ID NO: 3.

In some embodiments of the first aspect of the present invention, a ligand-camptothecin derivative conjugate shown in general formula I, or a pharmaceutically acceptable salt or solvate thereof, is disclosed, characterized in that the κ light chain of the Ab antibody comprises CDRs as shown in SEQ ID NO: 41, SEQ ID NO: 42, and SEQ ID NO: 43, the IgG1 heavy chain comprises CDRs as shown in SEQ ID NO: 45, SEQ ID NO: 46, and SEQ ID NO: 47, and the single-chain Fv (scFv) structural domain comprises heavy chain variable region CDRs as shown in SEQ ID NO: 32, SEQ ID NO: 33, and SEQ ID NO: 34, and light chain variable region CDRs as shown in SEQ ID NO: 35, SEQ ID NO: 36, and SEQ ID NO: 37.

In some embodiments of the first aspect of the present invention, the ligand-camptothecin derivative conjugate shown in general formula I, or a pharmaceutically acceptable salt or solvate thereof, is disclosed, characterized in that the light chain of the Ab antibody comprises a variable region as shown in SEQ ID NO: 44, the IgG1 heavy chain comprises a variable region as shown in SEQ ID NO: 48, and the single-chain Fv (scFv) structural domain comprises a heavy chain variable region as shown in SEQ ID NO: 39 and a light chain variable region as shown in SEQ ID NO: 40.

In some embodiments of the first aspect of the present invention, a ligand-camptothecin derivative conjugate shown in general formula I, or a pharmaceutically acceptable salt or solvate thereof, is disclosed, characterized in that the light chain amino acid sequence of the Ab antibody is SEQ ID NO: 6, and the amino acid sequence of the construct of the heavy chain of the antibody and the single-chain Fv (scFv) structural domain is SEQ ID NO: 8.

In some embodiments of the first aspect of the present invention, the ligand-camptothecin derivative conjugate shown in general formula I, or a pharmaceutically acceptable salt or solvate thereof, is disclosed, characterized in that the light chain nucleic acid coding sequence of the Ab antibody is SEQ ID NO: 5, and the nucleic acid coding sequence of the construct of the antibody heavy chain and the single-chain Fv (scFv) structural domain is SEQ ID NO: 7.

In some embodiments of the first aspect of the present invention, the ligand-camptothecin derivative conjugate shown in general formula I, or a pharmaceutically acceptable salt or solvate thereof, is disclosed, characterized in that the light chain of the Ab antibody comprises a variable region as shown in SEQ ID NO: 49, the IgG1 heavy chain comprises a variable region as shown in SEQ ID NO: 52, and a single-chain Fv (scFv) structural domain comprises a heavy chain variable region as shown in SEQ ID NO: 50 and a light chain variable region as shown in SEQ ID NO: 51.

In some embodiments of the first aspect of the present invention, a ligand-camptothecin derivative conjugate shown in general formula I, or a pharmaceutically acceptable salt or solvate thereof, is disclosed, characterized in that an Ab antibody has a heavy chain amino acid sequence of SEQ ID NO: 12, and a construct of the antibody light chain and a single-chain Fv (scFv) structural domain has an amino acid sequence of SEQ ID NO: 10.

In some embodiments of the first aspect of the present invention, a ligand-camptothecin derivative conjugate shown in general formula I, or a pharmaceutically acceptable salt or solvate thereof, is disclosed, characterized in that the Ab antibody has a heavy chain nucleic acid coding sequence of SEQ ID NO: 11, and a construct of the light chain of the antibody and a single-chain Fv (scFv) structural domain has a nucleic acid coding sequence of SEQ ID NO: 9.

In some embodiments of the first aspect of the present invention, the ligand-camptothecin derivative conjugate shown in general formula I, or a pharmaceutically acceptable salt or solvate thereof, is disclosed, characterized in that the Ab antibody has a light chain amino acid sequence of SEQ ID NO: 14, and a construct of the antibody heavy chain and a single-chain Fv (scFv) structural domain has an amino acid sequence of SEQ ID NO: 16.

In some embodiments of the first aspect of the present invention, the ligand-camptothecin derivative conjugate shown in general formula I, or a pharmaceutically acceptable salt or solvate thereof, is disclosed, characterized in that the Ab antibody has a light chain nucleic acid coding sequence of SEQ ID NO: 13, and a construct of the antibody heavy chain and a single-chain Fv (scFv) structural domain has a nucleic acid coding sequence of SEQ ID NO: 15.

In some embodiments of the first aspect of the present invention, the ligand-camptothecin derivative conjugate shown in general formula I, or a pharmaceutically acceptable salt or solvate thereof, is disclosed, characterized in that the Ab antibody has a heavy chain amino acid sequence of SEQ ID NO: 20, and a construct of the antibody light chain and a single-chain Fv (scFv) structural domain has an amino acid sequence of SEQ ID NO: 18.

In some embodiments of the first aspect of the present invention, the ligand-camptothecin derivative conjugate shown in general formula I, or a pharmaceutically acceptable salt or solvate thereof, is disclosed, characterized in that the Ab antibody has a heavy chain nucleic acid coding sequence of SEQ ID NO: 19, and a construct of the light chain of the antibody and a single-chain Fv (scFv) structural domain has a nucleic acid coding sequence of SEQ ID NO: 17.

In some embodiments of the first aspect of the present invention, the ligand-camptothecin derivative conjugate shown in general formula I, or a pharmaceutically acceptable salt or solvate thereof, is disclosed, characterized in that the light chain of the Ab antibody comprises a variable region as shown in SEQ ID NO: 53, an IgG1 heavy chain comprises a variable region as shown in SEQ ID NO: 54, and a single-chain Fv (scFv) structural domain comprises a heavy chain variable region as shown in SEQ ID NO: 50 and a light chain variable region as shown in SEQ ID NO: 51.

In some embodiments of the first aspect of the present invention, the ligand-camptothecin derivative conjugate shown in general formula I, or a pharmaceutically acceptable salt or solvate thereof, is disclosed, characterized in that the Ab antibody has a heavy chain amino acid sequence of SEQ ID NO: 24, and the amino acid sequence of a construct of the light chain of the antibody and a single-chain Fv (scFv) structural domain is SEQ ID NO: 22.

In some embodiments of the first aspect of the present invention, the ligand-camptothecin derivative conjugate shown in general formula I, or a pharmaceutically acceptable salt or solvate thereof, is disclosed, characterized in that the Ab antibody has a heavy chain nucleic acid coding sequence of SEQ ID NO: 23, and a construct of the antibody light chain and a single-chain Fv (scFv) structural domain has a nucleic acid coding sequence of SEQ ID NO: 21.

In some embodiments of the first aspect of the present invention, a ligand-camptothecin derivative conjugate shown in general formula I, or a pharmaceutically acceptable salt or solvate thereof, is disclosed, characterized in that the Ab antibody comprises: two IgG1 heavy chains; two κ light chains; and two single-chain Fv (scFv) structural domains.

In some embodiments of the first aspect of the present invention, ligand-camptothecin derivative conjugates as shown in general formula I or pharmaceutically acceptable salts or solvates thereof are disclosed, characterized in that said X is selected without limitation from the following structures or isomers thereof:

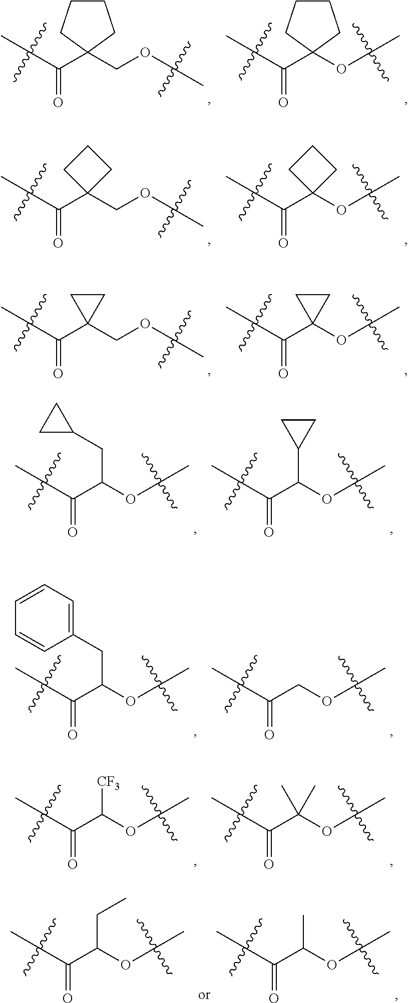

where the left wavy line is linked to a camptothecin derivative portion and the right wavy line is linked to L₅.

In some embodiments of the first aspect of the present invention, ligand-camptothecin derivative conjugates as shown in general formula I or pharmaceutically acceptable salts or solvates thereof are disclosed, characterized in that L₄ is selected without limitation from peptide residues formed of amino acids;
wherein optionally, said amino acid is further substituted with one or more substituents selected from deuterium atoms, halogens, hydroxyl, cyano, amino, nitro, carboxyl, C1-C6 alkyl, substituted C1-C6 alkyl, C1-C6 alkoxy and C3-C8 cycloalkyl or substituted C3-C8 cycloalkyl;
preferably, said peptide residue is a peptide residue formed from one, two or more amino acids selected from phenylalanine (F), glycine (G), valine (V), lysine (K), citrulline (C), serine (S), glutamic acid (E) or aspartic acid (D);
more preferably, said peptide residue is a tetrapeptide residue consisting of glycine (G)-glycine (G)-phenylalanine (F)-glycine (G).

Particularly preferably, said peptide residue is -GGFG-.

In some embodiments of the first aspect of the present invention, ligand-camptothecin derivative conjugates as shown in general formula I or pharmaceutically acceptable salts or solvates thereof are disclosed, characterized as follows:
L₅ is non-limitatively selected from —NR₅(CR₆R₇)_{q}— or a chemical bond, q is selected from an integer from 0-6 (e.g., 0, 1, 2, 3, 4, 5, or 6);
R₅, R₆ and R₇ are identical or different and are each independently selected from a hydrogen atom, a deuterium atom, a halogen, a C1-C6 alkyl group, a substituted C1-C6 alkyl group, a deuterated C1-C6 alkyl group, a C3-C8 cycloalkyl group, a C3-C8 cycloalkyl C1-C6 alkyl group, a C1-C6 alkoxy C1-C6 alkyl group, a 3-7-membered heterocyclyl group, a substituted 3-7-membered heterocyclyl group, a C6-C10 aryl group, a substituted C6-C10 aryl, 5-10-membered heteroaryl, substituted 5-10-membered heteroaryl;
preferably, R₅, R₆ and R₇ are each independently selected from a hydrogen atom or a C1-C6 alkyl group;
more preferably, R₅, R₆ and R₇ are each independently selected from a hydrogen atom.

In certain embodiments, L₁ is selected without limitation from:

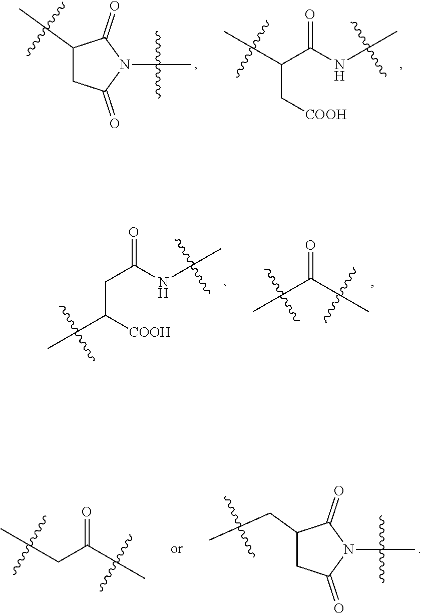

In some embodiments of the first aspect of the present invention, ligand-camptothecin derivative conjugates as shown in general formula I or pharmaceutically acceptable salts or solvates thereof are disclosed, characterized in that said linking unit -L₁-L₂-L₃-L₄-L₅- is selected without limitation from the following structures;

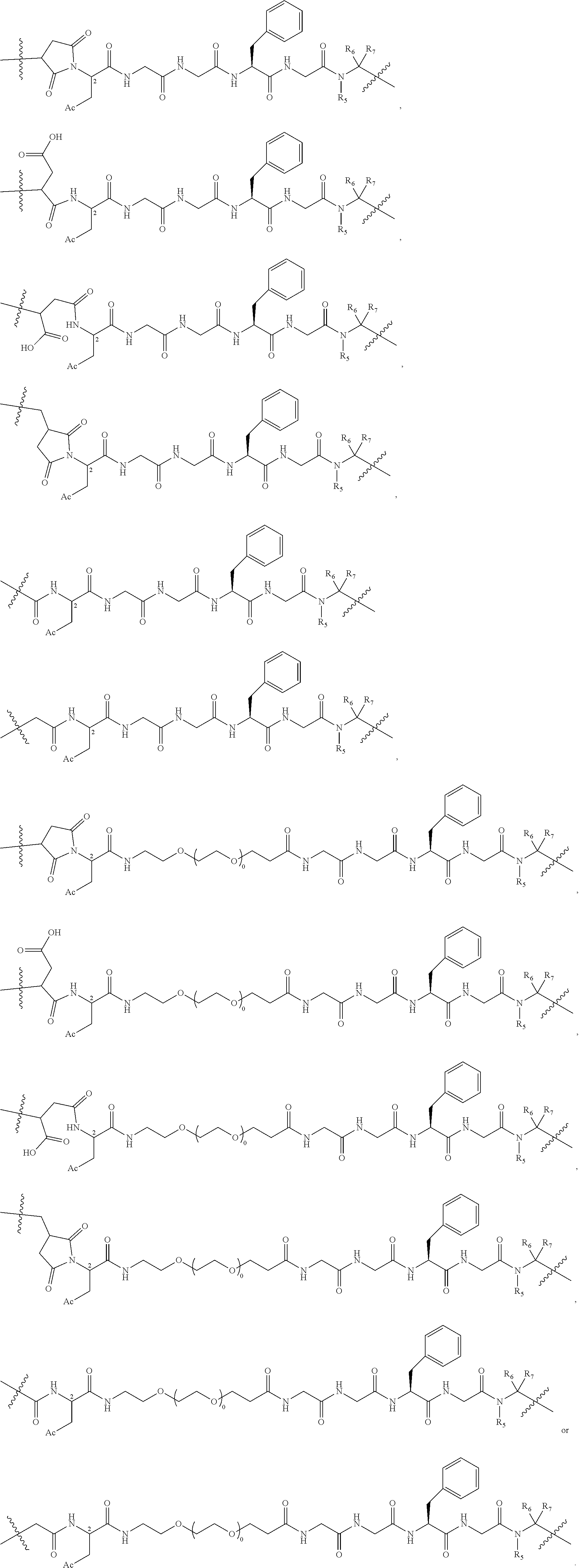

preferably

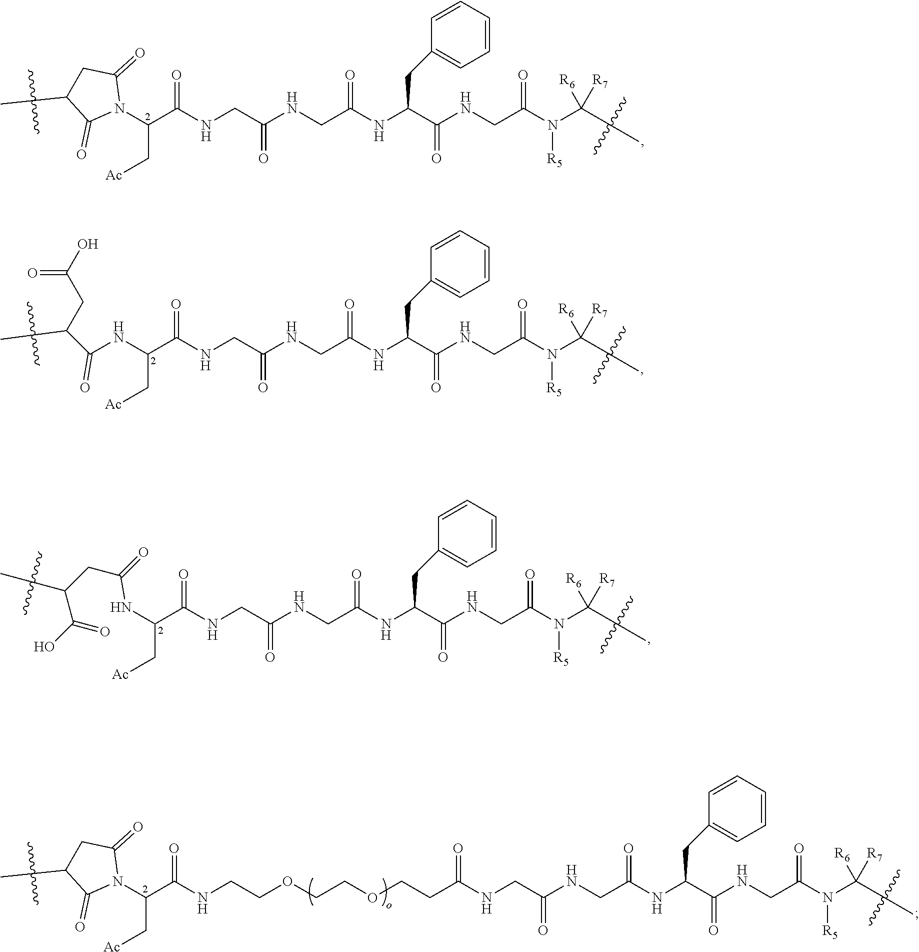

wherein:
Ac is a hydrophilic structural unit;
R₅, R₆ and R₇ are identical or different and are each independently selected from hydrogen atom, deuterium atom, halogen, C1-C6 alkyl, substituted C1-C6 alkyl, deuterated C1-C6 alkyl, C3-C8 cycloalkyl, C3-C8 cycloalkyl C1-C6 alkyl, C1-C6 alkoxy C1-C6 alkyl, 3-7-membered heterocyclyl, substituted 3-7-membered heterocyclyl, C6-C10 aryl, substituted C6-C10 aryl, 5-10-membered heteroaryl, substituted 5-10-membered heteroaryl;
preferably, R₅, R₆ and R₇ are each independently selected from a hydrogen atom or a C1-C6 alkyl group;
more preferably, R₅, R₆ and R₇ are each independently selected from a hydrogen atom;
the carbon atom No. 2 attached to N has absolute chirality in the R configuration or the S configuration;
the left wavy line is linked to the antibody or its antigen-binding fragment portion, and the right wavy line is connected to the X;
is selected from integers 1-10 (i.e., 1, 2, 3, 4, 5, 6, 7, 8, 9, or 10).

The second aspect of the present invention discloses a ligand-camptothecin derivative conjugate as shown in general formula II, or a pharmaceutically acceptable salt or solvate thereof;

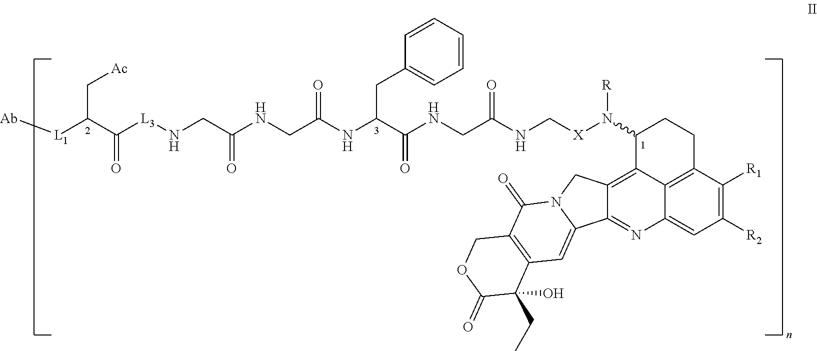

wherein:
Ab is a bispecific antibody or antigen-binding fragment thereof that simultaneously targets EGFR and HER3;
L₁ is a linking unit that is connected to Ab, selected without limitation from:

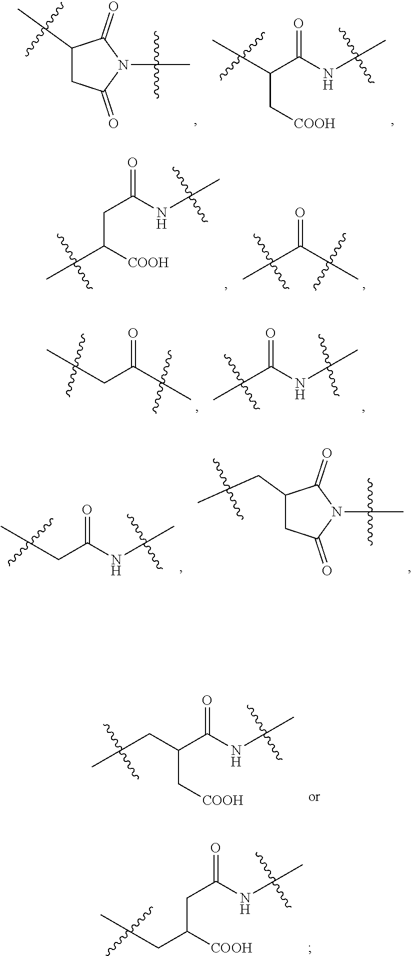

preferably, L₁ is:

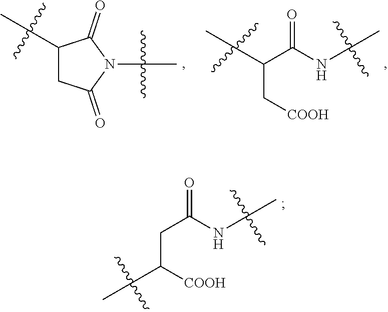 [SMILES: *C1CC(=O)N(*)C1=O ; *NC(=O)C(*)CC(=O)O ; *NC(=O)CC(*)C(=O)O]

preferably, L₁ is:

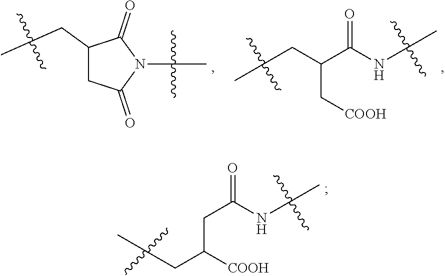 [SMILES: *CC1CC(=O)N(*)C1=O ; *CC(CC(=O)O)C(=O)N* ; *CC(CC(=O)N*)C(=O)O]

L₃ is present or absent, and when L₃ is present, L₃ is selected from

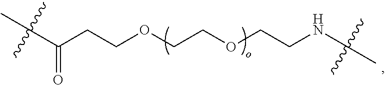

o is selected from integers 1-10 (e.g., 1, 2, 3, 4, 5, 6, 7, 8, 9, or 10), preferably integers 2-8;
Ac is a hydrophilic structural unit;
the chiral carbon atoms at position 1, position 2 and position 3 have two chiral configurations, namely the R absolute configuration or the S absolute configuration;
R is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, C6-C10 aryl, substituted C6-C10 aryl, 5-10-membered heteroaryl, and substituted 5-10-membered heteroaryl;
preferably R is selected from a hydrogen atom or a C1-C6 alkyl group;
R₁ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7-membered heterocyclyl, substituted 3-7-membered heterocyclyl, C6-C10 aryl, substituted C6-C10 aryl, 5-10-membered heteroaryl, substituted 5-10-membered heteroaryl;
preferably R₁ is selected from a hydrogen atom or a C1-C6 alkyl group;
more preferably, R₁ is selected from C1-C6 alkyl;
R₂ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7-membered heterocyclyl, substituted 3-7-membered heterocyclyl, C6-C10 aryl, substituted C6-C10 aryl, 5-10-membered heteroaryl, substituted 5-10-membered heteroaryl;
preferably R₂ is selected from a hydrogen atom, a halogen or a C1-C6 alkyl group;
more preferably R₂ is selected from halogen;
X is selected from —C(O)—CRₐR_{b}—(CR₃R₄)ₘ—O—, —C(O)—CRₐR_{b}—(CR₃R₄)ₘ—NH— or —C(O)—CRₐR_{b}—(CR₃R₄)ₘ—S—;
preferably X is selected from —C(O)—CRₐR_{b}—(CR₃R₄)ₘ—O—;
Rₐ and R_{b} are each independently selected from a hydrogen atom, a deuterium atom, a halogen, a C1-C6 alkyl group, a deuterated C1-C6 alkyl group, a halogenated C1-C6 alkyl group, a C3-C8 cycloalkyl group, a C3-C8 cycloalkyl C1-C6 alkyl group, a C1-C6 alkoxy C1-C6 alkyl group, a 3-7-membered heterocyclic group, a substituted 3-7-membered heterocyclic group, a C6-C10 aryl group, a substituted C6-C10 aryl group, a 5-10-membered heteroaryl, a substituted 5-10-membered heteroaryl;
preferably, Rₐ and R_{b} are each independently selected from a hydrogen atom, a C1-C6 alkyl, a halo-C1-C6 alkyl, a C3-C8 cycloalkyl C1-C6 alkyl or a C6-C10 aryl C1-C6 alkyl;
alternatively, Rₐ, R_{b} and the carbon atoms to which they are attached constitute a C3-C8 cycloalkyl group, a C3-C8 cycloalkyl C1-C6 alkyl group, a 3-7-membered heterocyclyl group, a substituted 3-7-membered heterocyclyl group; preferably Rₐ, R_{b} and the carbon atoms to which they are attached constitute a C3-C8 cycloalkyl group;
R₃, R₄ are identical or different and are independently hydrogen atoms, deuterium atoms, halogens, C1-C6 alkyl, halogenated C1-C6 alkyl, deuterated C1-C6 alkyl, C1-C6 alkoxy, hydroxyl, amino, cyano, nitro, hydroxy C1-C6 alkyl, C3-C8 cycloalkyl, 3-7-membered heterocyclyl, substituted 3-7-membered heterocyclyl, respectively;
preferably, R₃, R₄ are independently hydrogen atoms or C1-C6 alkyl groups, respectively;
alternatively, R₃, R₄ and the carbon atoms attached thereto constitute a C3-C8 cycloalkyl group, a C3-C8 cycloalkyl C1-C6 alkyl group, a 3-7-membered heterocyclic group, a substituted 3-7-membered heterocyclic group;
m is selected from integers 0-4 (i.e., 0, 1, 2, 3 or 4), preferably 0, 1;
n is selected from the integers 1-10 (e.g., 1, 2, 3, 4, 5, 6, 7, 8, 9, or 10).

In some embodiments of the first and second aspects of the present invention, it is disclosed that said ligand-camptothecin derivative conjugate, or a pharmaceutically acceptable salt or solvate thereof, is characterized in that: said Ac has the structure shown in formula B, as follows,

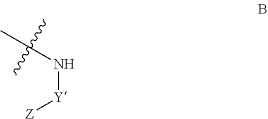

wherein:
Z is selected without limitation from the group consisting of one or more of a hydrophilic structural carboxyl group, phosphoric acid, polyphosphoric acid, phosphorous acid, sulfonic acid, sulfinic acid, or polyethylene glycol (PEG);
preferably Z is selected from a hydrophilic structural carboxyl group, phosphoric acid or polyethylene glycol (PEG);
Y′ is optionally a scaffold connecting the amino group to Z; preferably Y′ is a C1-C6 alkylene group (e.g. methylene);
Ac is connected to the 2-position carbon that has been labeled in structural formula I by means of a scaffold Y.

In some embodiments of the first aspect and the second aspect of the present invention, it is disclosed that said ligand-camptothecin derivative conjugate, or a pharmaceutically acceptable salt or solvate thereof, is characterized in that: said Ac is selected without limitation from glycine, (D/L) alanine, (D/L) leucine, (D/L) isoleucine, (D/L) valine, (D/L) phenylalanine, (D/L) proline, (D/L) tryptophan, (D/L) serine, (D/L) tyrosine, (D/L) cysteine, (D/L) cystine, (D/L) arginine, (D/L) histidine, (D/L) methionine, (D/L) asparagine, (D/L) glutamine, (D/L) threonine, (D/L) aspartic acid, (D/L) glutamic acid, natural or unnatural amino acid derivatives or the following structures or isomers thereof,

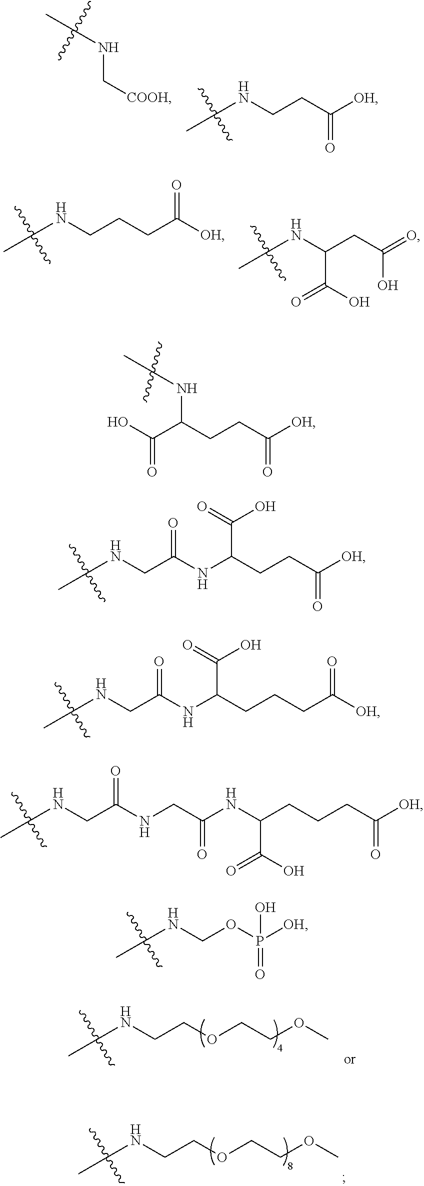

preferably

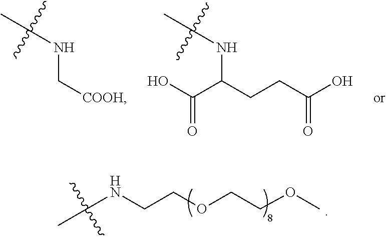

In some embodiments of the first and second aspects of the present invention, the disclosure of said ligand-camptothecin derivative conjugates or pharmaceutically acceptable salts or solvates thereof is characterized in that: said Ac is selected without limitation from a glycine, phosphoric acid, (D/L) glutamic acid, or polyethylene glycol hydrophilic structure.

In some embodiments of the first and second aspects of the present invention, the disclosure of said ligand-camptothecin derivative conjugates or pharmaceutically acceptable salts or solvates thereof is characterized in that said

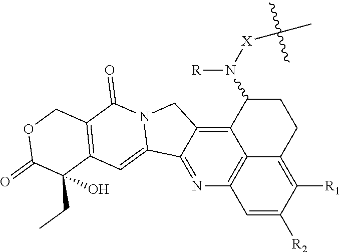

has the structure shown in formula d below;

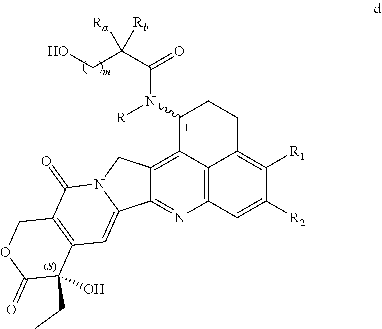

wherein:
R is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, C6-C10 aryl, substituted C6-C10 aryl, 5-10-membered heteroaryl, substituted 5-10-membered heteroaryl;
preferably R is selected from a hydrogen atom or a C1-C6 alkyl group;
R₁ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7-membered heterocyclyl, substituted 3-7-membered heterocyclyl, C6-C10 aryl, substituted C6-C10 aryl, 5-10-membered heteroaryl, substituted 5-10-membered heteroaryl;
preferably R₁ is selected from a hydrogen atom or a C1-C6 alkyl group;
more preferably R₁ is selected from C1-C6 alkyl;
R₂ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7-membered heterocyclyl, substituted 3-7-membered heterocyclyl, C6-C10 aryl, substituted C6-C10 aryl, 5-10-membered heteroaryl, substituted 5-10-membered heteroaryl;
preferably R₂ is selected from a hydrogen atom, a halogen or a C1-C6 alkyl group;
more preferably R₂ is selected from halogen;
Rₐ and R_{b} are each independently selected from a hydrogen atom, a deuterium atom, a halogen, a C1-C6 alkyl, a deuterated C1-C6 alkyl, a halogenated C1-C6 alkyl, a C3-C8 cycloalkyl, a C3-C8 cycloalkyl C1-C6 alkyl, a C1-C6 alkoxy C1-C6 alkyl, a 3-7-membered heterocyclic group, a substituted 3-7-membered heterocyclic group, a C6-C10 aryl, a substituted C6-C10 aryl, a 5-10-membered heteroaryl, a substituted 5-10-membered heteroaryl;
preferably, Rₐ and R_{b} are each independently selected from a hydrogen atom, a C1-C6 alkyl, a halo-C1-C6 alkyl, a C3-C8 cycloalkyl C1-C6 alkyl or a C6-C10 aryl C1-C6 alkyl;
preferably Rₐ and R_{b} are each independently selected from a hydrogen atom, a C1-C6 alkyl, a halo-C1-C6 alkyl, a C3-C8 cycloalkyl C1-C6 alkyl, a C6-C10 aryl; preferably Rₐ and R_{b} are each independently selected from a hydrogen atom, a methyl group, an ethyl group, a trifluoromethyl group, a cyclopropylmethyl group, a phenyl group;
alternatively, Rₐ, R_{b} and the carbon atoms to which they are attached constitute a C3-C8 cycloalkyl group, a C3-C8 cycloalkyl C1-C6 alkyl group, a 3-7-membered heterocyclyl group, a substituted 3-7-membered heterocyclyl group; preferably, Rₐ, R_{b} and the carbon atoms to which they are attached constitute a C3-C8 cycloalkyl group (e.g., a C3-C5 cycloalkyl group);
the 1-position chiral carbon atom has two chiral configurations, namely the R absolute configuration or the S absolute configuration;
m is selected from 0 or 1.

In some embodiments of the first and second aspects of the present invention, the disclosure of said ligand-camptothecin derivative conjugate or a pharmaceutically acceptable salt or solvate thereof is characterized in that: said structural formula d is selected without limitation from the following compounds:

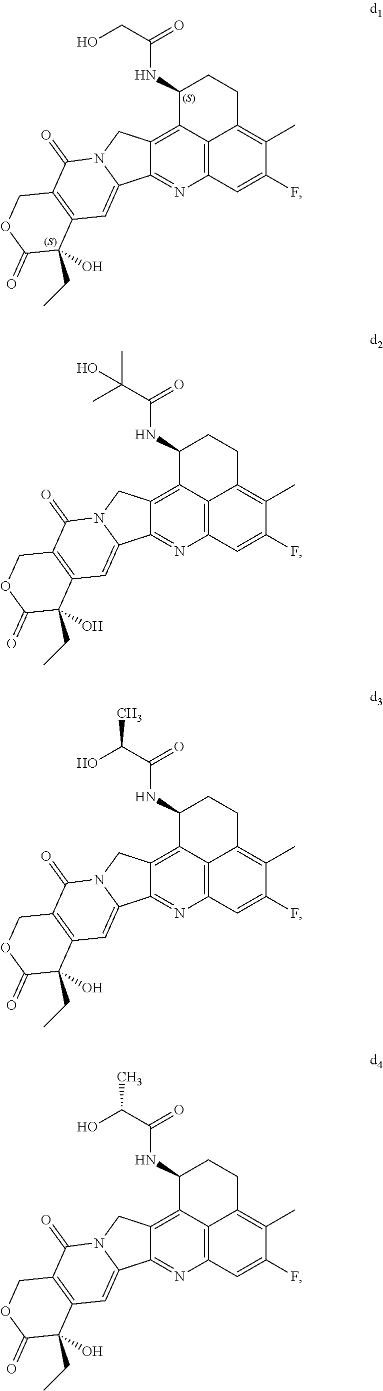

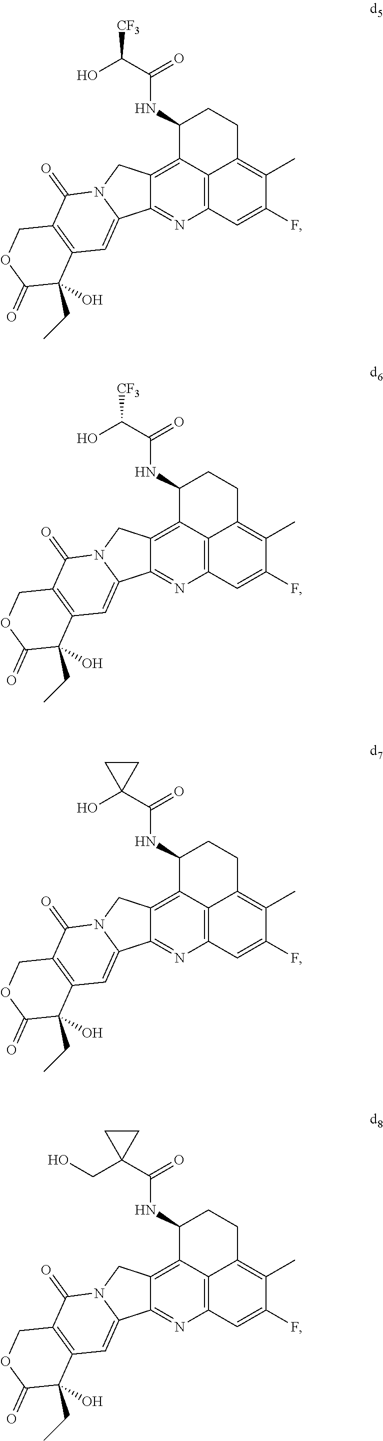 [SMILES: CC[C@@]1(O)C(=O)OCc2c1cc1n(c2=O)Cc2c-1nc1cc(F)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(C)c3c1c2[C@@H](NC(=O)C1(O)CC1)CC3 ; CC[C@@]1(O)C(=O)OCc2c1cc1n(c2=O)Cc2c-1nc1cc(F)c(C)c3c1c2[C@@H](NC(=O)C1(CO)CC1)CC3]

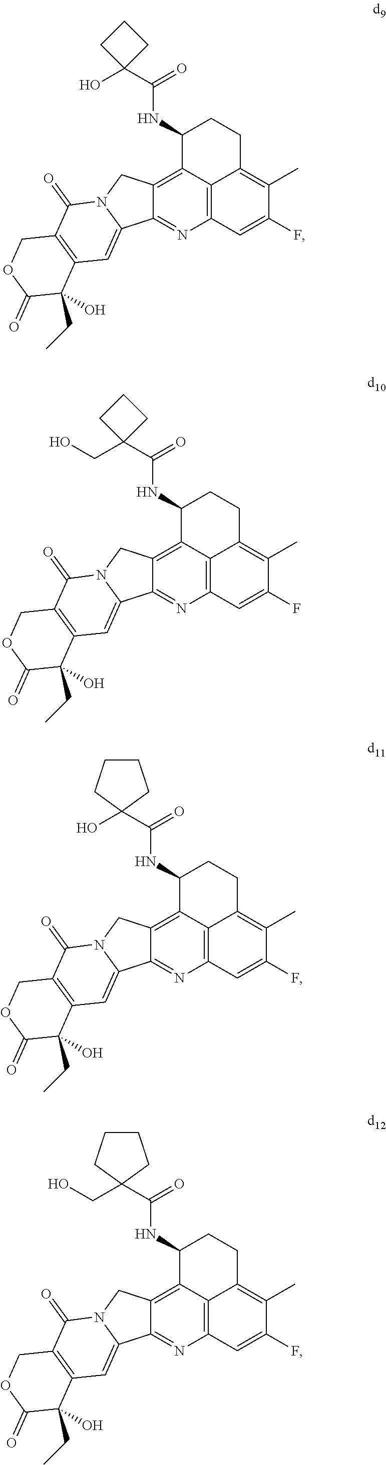 [SMILES: CC[C@@]1(O)C(=O)OCc2c1cc1n(c2=O)Cc2c-1nc1cc(F)c(C)c3c1c2[C@@H](NC(=O)C1(O)CCC1)CC3 ; CC[C@@]1(O)C(=O)OCc2c1cc1n(c2=O)Cc2c-1nc1cc(F)c(C)c3c1c2[C@@H](NC(=O)C1(CO)CCC1)CC3 ; CC[C@@]1(O)C(=O)OCc2c1cc1n(c2=O)Cc2c-1nc1cc(F)c(C)c3c1c2[C@@H](NC(=O)C1(O)CCCC1)CC3 ; CC[C@@]1(O)C(=O)OCc2c1cc1n(c2=O)Cc2c-1nc1cc(F)c(C)c3c1c2[C@@H](NC(=O)C1(CO)CCCC1)CC3]

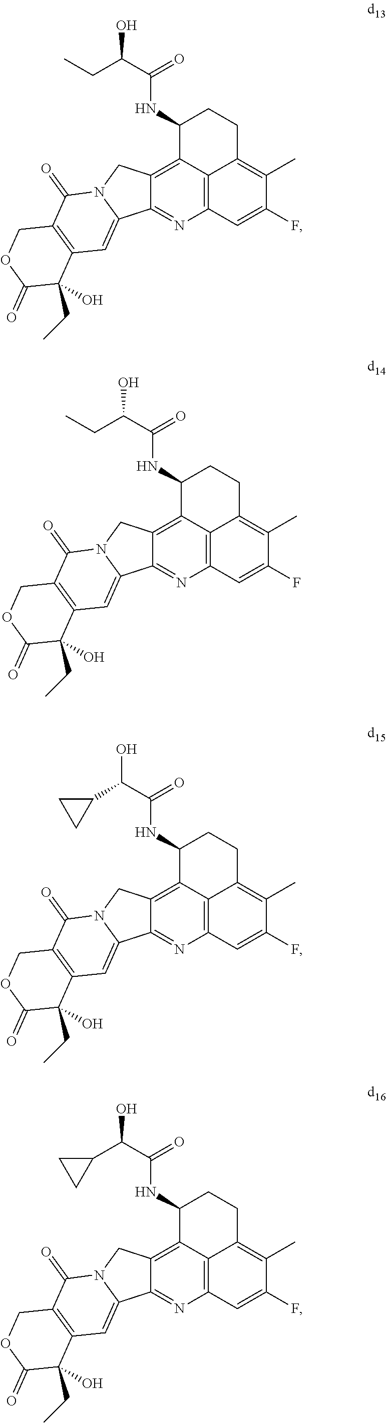 [SMILES: CC[C@@H](O)C(=O)N[C@H]1CCc2c(C)c(F)cc3nc4c(c1c23)Cn1c-4cc2c(c1=O)COC(=O)[C@]2(O)CC ; CC[C@H](O)C(=O)N[C@H]1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)[C@@H](O)C1CC1)CC3 ; CC[C@@]1(O)C(=O)OCc2c1cc1n(c2=O)Cc2c-1nc1cc(F)c(C)c3c1c2[C@@H](NC(=O)[C@H](O)C1CC1)CC3]

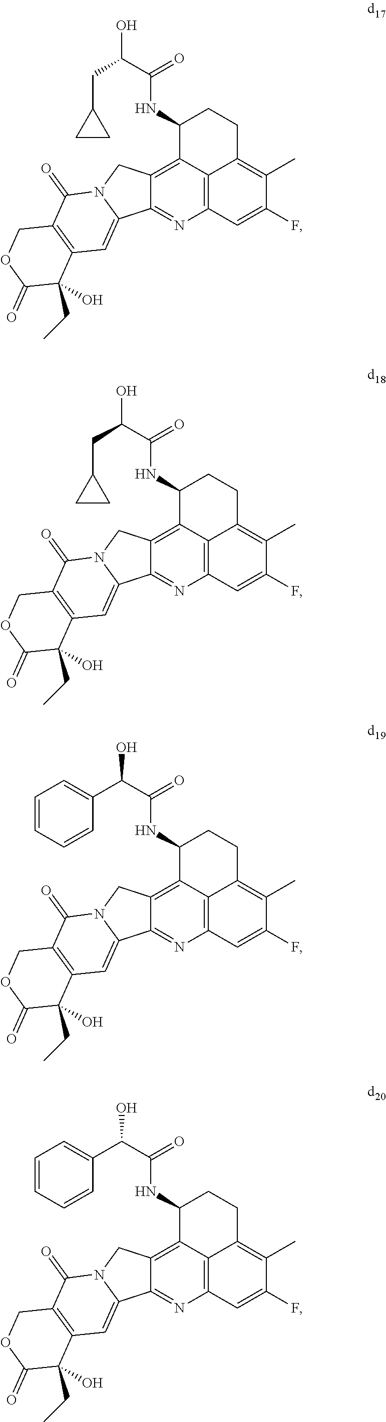 [SMILES: CC[C@@]1(O)C(=O)OCc2c1cc1n(c2=O)Cc2c-1nc1cc(F)c(C)c3c1c2[C@@H](NC(=O)[C@@H](O)CC1CC1)CC3 ; CC[C@@]1(O)C(=O)OCc2c1cc1n(c2=O)Cc2c-1nc1cc(F)c(C)c3c1c2[C@@H](NC(=O)[C@H](O)CC1CC1)CC3 ; CC[C@@]1(O)C(=O)OCc2c1cc1n(c2=O)Cc2c-1nc1cc(F)c(C)c3c1c2[C@@H](NC(=O)[C@H](O)c1ccccc1)CC3 ; CC[C@@]1(O)C(=O)OCc2c1cc1n(c2=O)Cc2c-1nc1cc(F)c(C)c3c1c2[C@@H](NC(=O)[C@@H](O)c1ccccc1)CC3]

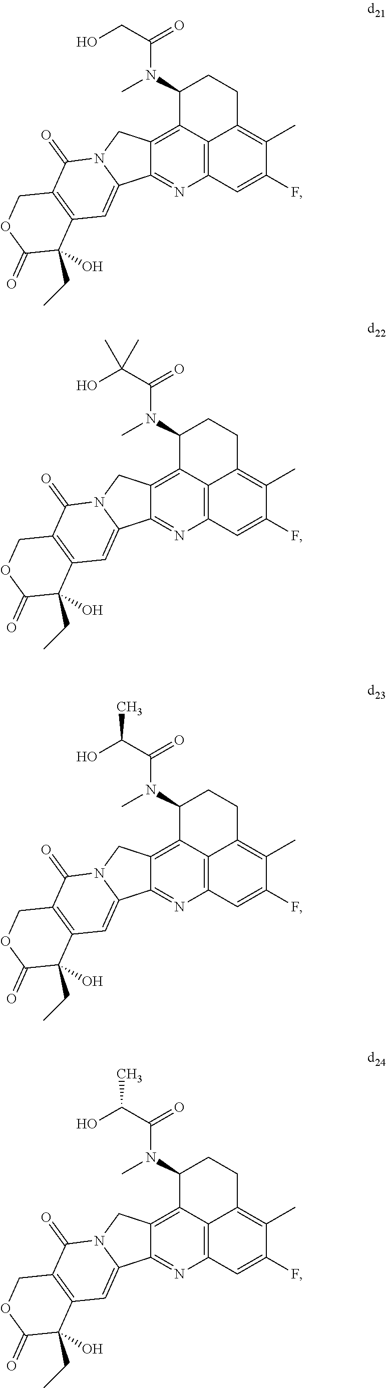 [SMILES: CC[C@@]1(O)C(=O)OCc2c1cc1n(c2=O)Cc2c-1nc1cc(F)c(C)c3c1c2[C@@H](N(C)C(=O)CO)CC3 ; CC[C@@]1(O)C(=O)OCc2c1cc1n(c2=O)Cc2c-1nc1cc(F)c(C)c3c1c2[C@@H](N(C)C(=O)C(C)(C)O)CC3 ; CC[C@@]1(O)C(=O)OCc2c1cc1n(c2=O)Cc2c-1nc1cc(F)c(C)c3c1c2[C@@H](N(C)C(=O)[C@H](C)O)CC3 ; CC[C@@]1(O)C(=O)OCc2c1cc1n(c2=O)Cc2c-1nc1cc(F)c(C)c3c1c2[C@@H](N(C)C(=O)[C@@H](C)O)CC3]

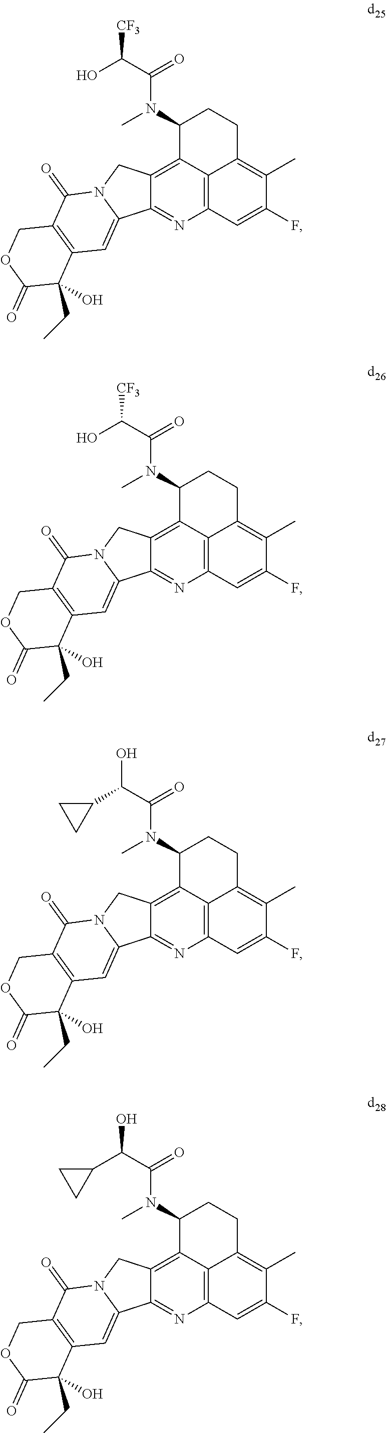 [SMILES: CC[C@@]1(O)C(=O)OCc2c1cc1n(c2=O)Cc2c-1nc1cc(F)c(C)c3c1c2[C@@H](N(C)C(=O)[C@@H](O)C(F)(F)F)CC3 ; CC[C@@]1(O)C(=O)OCc2c1cc1n(c2=O)Cc2c-1nc1cc(F)c(C)c3c1c2[C@@H](N(C)C(=O)[C@H](O)C(F)(F)F)CC3 ; CC[C@@]1(O)C(=O)OCc2c1cc1n(c2=O)Cc2c-1nc1cc(F)c(C)c3c1c2[C@@H](N(C)C(=O)[C@@H](O)C1CC1)CC3 ; CC[C@@]1(O)C(=O)OCc2c1cc1n(c2=O)Cc2c-1nc1cc(F)c(C)c3c1c2[C@@H](N(C)C(=O)[C@H](O)C1CC1)CC3]

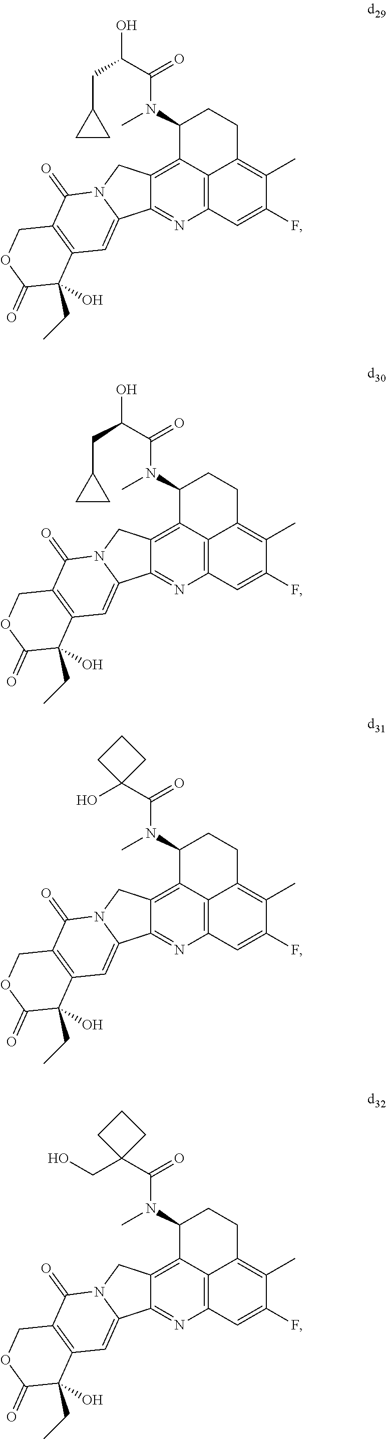 [SMILES: CC[C@@]1(O)C(=O)OCc2c1cc1n(c2=O)Cc2c-1nc1cc(F)c(C)c3c1c2[C@@H](N(C)C(=O)[C@@H](O)CC1CC1)CC3 ; CC[C@@]1(O)C(=O)OCc2c1cc1n(c2=O)Cc2c-1nc1cc(F)c(C)c3c1c2[C@@H](N(C)C(=O)[C@H](O)CC1CC1)CC3 ; CC[C@@]1(O)C(=O)OCc2c1cc1n(c2=O)Cc2c-1nc1cc(F)c(C)c3c1c2[C@@H](N(C)C(=O)C1(O)CCC1)CC3 ; CC[C@@]1(O)C(=O)OCc2c1cc1n(c2=O)Cc2c-1nc1cc(F)c(C)c3c1c2[C@@H](N(C)C(=O)C1(CO)CCC1)CC3]

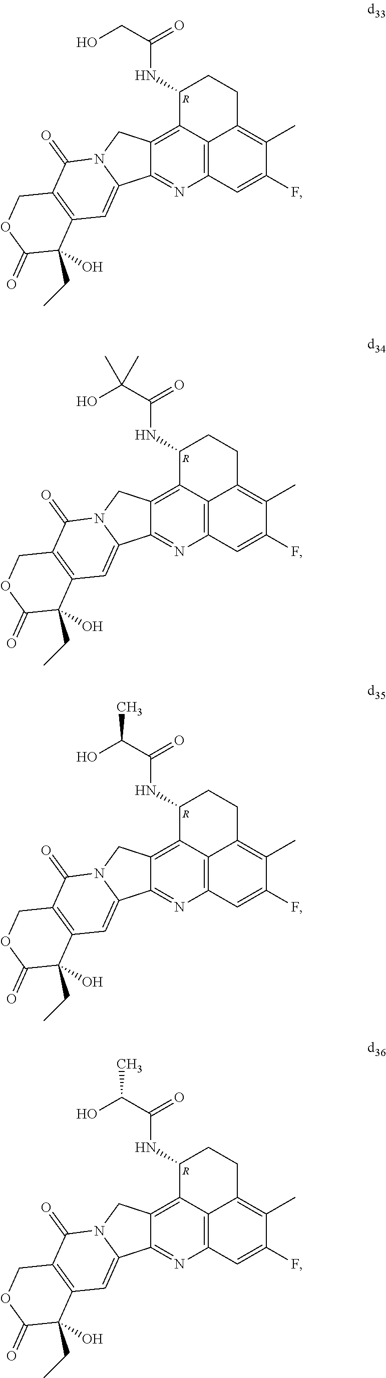

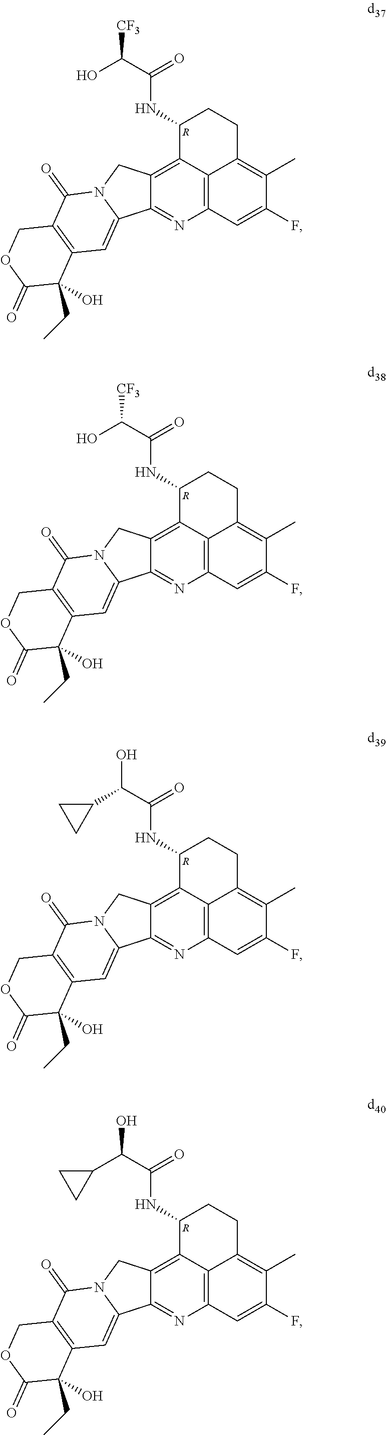

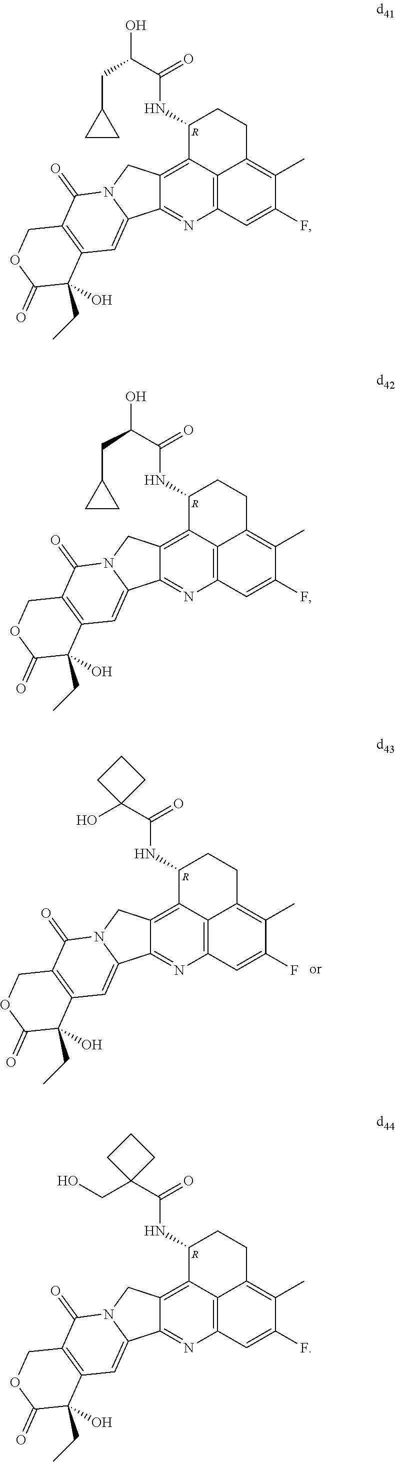

In some embodiments of the present invention, L₁ can comprise a succinimide group. In these embodiments, the ligand-drug conjugate can undergo hydrolysis under readily hydrolyzable conditions, with the site of hydrolysis being the succinimide group of the linker unit. When the ligand contains multiple linker-drugs, the following scenarios can occur with varying degrees of hydrolysis:
the succinimide groups are completely non-hydrolyzed, i.e., the succinimide groups are all in a closed ring form

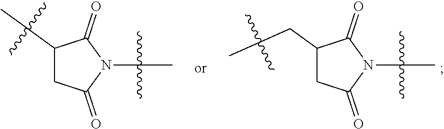 [SMILES: *C1CC(=O)N(*)C1=O ; *CC1CC(=O)N(*)C1=O]

incomplete hydrolysis of the succinimide groups, i.e., some of the succinimide groups are in a closed ring form

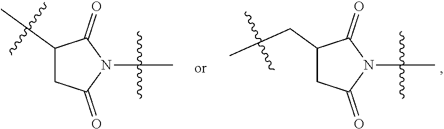 [SMILES: *C1CC(=O)N(*)C1=O ; *CC1CC(=O)N(*)C1=O]

and some of the succinimide groups are in an open ring form

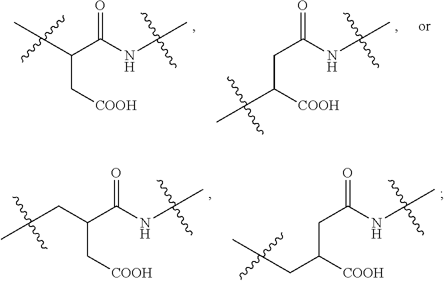 [SMILES: *NC(=O)C(*)CC(=O)O ; *NC(=O)CC(*)C(=O)O ; *CC(CC(=O)O)C(=O)N* ; *CC(CC(=O)N*)C(=O)O]

complete hydrolysis of the succinimide groups, i.e., the succinimide groups are all in an open ring form

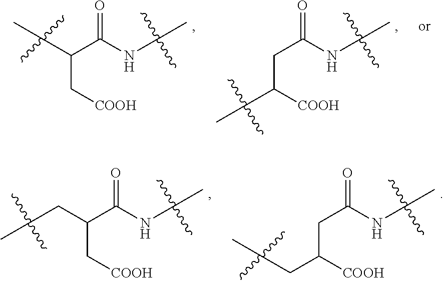 [SMILES: *NC(=O)C(*)CC(=O)O ; *NC(=O)CC(*)C(=O)O ; *CC(CC(=O)O)C(=O)N* ; *CC(CC(=O)N*)C(=O)O]

Thus, when multiple L₁ containing succinimide groups are present in the ADC at the same time (i.e., Ab is connected to multiple drug-linkers containing succinimide groups), these succinimide groups may be all in a closed ring form, partially in an open ring form, or all in an open ring form.

It will be appreciated that the present application, even though the succinimide group appearing in the chemical structural formula of the ADC is in the closed ring form, actually covers the three scenarios of the succinimide being all in a closed ring form, partially in an open ring form, and all in an open ring form. A third aspect of the present invention discloses a linker-drug compound or a pharmaceutically acceptable salt or solvate thereof, characterized by having the structure shown in formula III below,

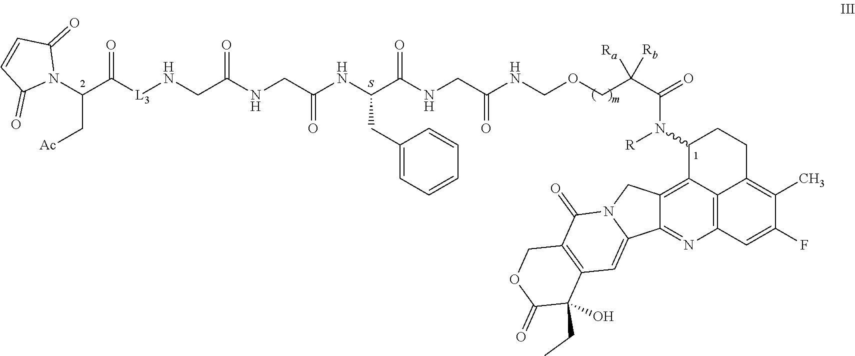

wherein:
R is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, C6-C10 aryl, substituted C6-C10 aryl, 5-10-membered heteroaryl, substituted 5-10-membered heteroaryl;
Rₐ is selected from hydrogen atom, deuterium atom, halogen, C1-C6 alkyl, deuterated C1-C6 alkyl, halogenated C1-C6 alkyl, C3-C8 cycloalkyl, C3-C8 cycloalkyl C1-C6 alkyl, C1-C6 alkoxy C1-C6 alkyl, 3-7-membered heterocyclic group, substituted 3-7 membered heterocyclic group, C6-C10 aryl, substituted C6-C10 aryl, 5-10 membered heteroaryl, substituted 5-10-membered heteroaryl;
R_{b} is selected from hydrogen atom, deuterium atom, halogen, C1-C6 alkyl, deuterated C1-C6 alkyl, halogenated C1-C6 alkyl, C3-C8 cycloalkyl, C3-C8 cycloalkyl C1-C6 alkyl, C1-C6 alkoxy C1-C6 alkyl, 3-7 membered heterocyclic group, substituted 3-7 membered heterocyclic group, C6-C10 aryl, substituted C6-C10 aryl, 5-10 membered heteroaryl, substituted 5-10-membered heteroaryl;
alternatively, Rₐ, R_{b} and the carbon atoms attached thereto constitute a C3-C8 cycloalkyl group, a C3-C8 cycloalkyl C1-C6 alkyl group, a 3-7-membered heterocyclic group, a substituted 3-7-membered heterocyclic group;
preferably Rₐ and R_{b} are each independently selected from a hydrogen atom, a C1-C6 alkyl, a halo-C1-C6 alkyl, a C3-C8 cycloalkyl C1-C6 alkyl, a C6-C10 aryl; preferably Rₐ and R_{b} are each independently selected from a hydrogen atom, a methyl group, an ethyl group, a trifluoromethyl group, a cyclopropylmethyl group, a phenyl group;
alternatively, Rₐ, R_{b} and the carbon atoms to which they are attached constitute a C3-C8 cycloalkyl group, a C3-C8 cycloalkyl C1-C6 alkyl group, a 3-7-membered heterocyclyl group, a substituted 3-7-membered heterocyclyl group; preferably, Rₐ, R_{b} and the carbon atoms to which they are attached constitute a C3-C8 cycloalkyl group (e.g., a C3-C5 cycloalkyl group);
L₃ is present or absent, and when L₃ is present, it is selected from

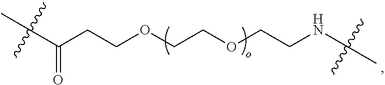

o is selected from an integer from 1 to 10 (e.g., 1, 2, 3, 4, 5, 6, 7, 8, 9, or 10);
the 1-position or 2-position chiral carbon atom has two chiralities, namely the R absolute configuration or S absolute configuration;
Ac is a hydrophilic structural unit;
m is selected from 0 or 1.

In some embodiments of the third aspect of the present invention, it is disclosed that said linker-drug compounds or pharmaceutically acceptable salts or solvates thereof are characterized in that: said Ac has the structure shown in formula B below,

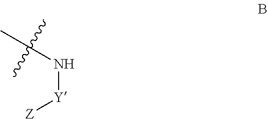

wherein:
Z is selected without limitation from the group consisting of one or more of a hydrophilic structural carboxyl group, a phosphoric acid, a polyphosphoric acid, a phosphorous acid, a sulfonic acid, a sulfinic acid, or a polyethylene glycol (PEG);
Y′ is an optional scaffold connecting the amino group to Z; preferably Y′ is a C1-C6 alkylene group (e.g. methylene);
Ac is connected to the 2-position carbon that has been labeled in structural formula I by means of scaffold Y.

In some embodiments of the third aspect of the present invention, disclosed are the linker-drug compounds or pharmaceutically acceptable salts or solvates thereof, characterized in that said Ac is selected without limitation from glycine, (D/L) alanine, (D/L) leucine, (D/L) isoleucine, (D/L) valine, (D/L) phenylalanine, (D/L) proline, (D/L) tryptophan, (D/L) serine, (D/L) tyrosine, (D/L) cysteine, (D/L) cystine, (D/L) arginine, (D/L) histidine, (D/L) methionine, (D/L) asparagine, (D/L) glutamine, (D/L) threonine, (D/L) aspartic acid, (D/L) glutamic acid, natural or unnatural amino acid derivatives, or the following structures,

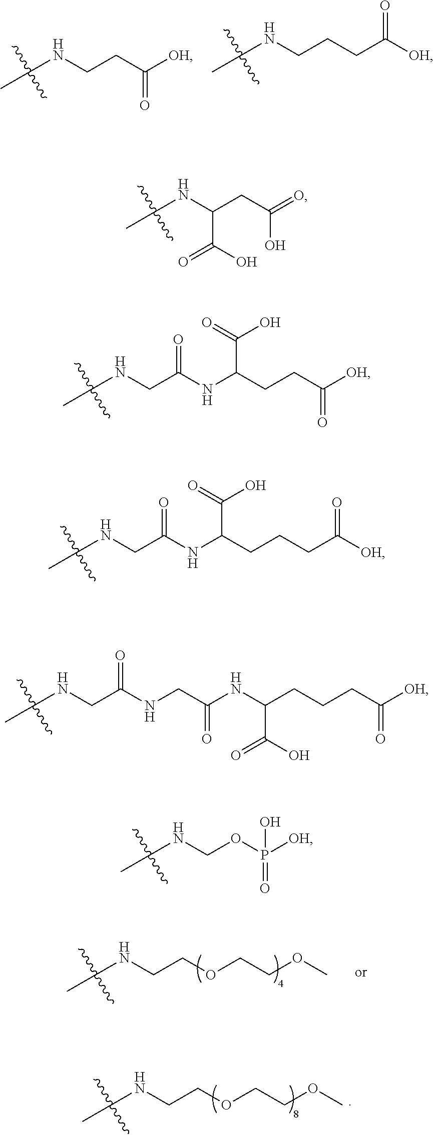

In some embodiments of the third aspect of the present invention, disclosed are the linker-drug compounds or pharmaceutically acceptable salts or solvates thereof, characterized in that: the Ac is selected without limitation from a glycine, phosphoric acid, (D/L) glutamic acid, or polyethylene glycol hydrophilic structure.

In some embodiments of the third aspect of the present invention, disclosed are the linker-drug compounds or pharmaceutically acceptable salts or solvates thereof, characterized in that said linker-drug compounds are selected without limitation from the following structures or isomers thereof,

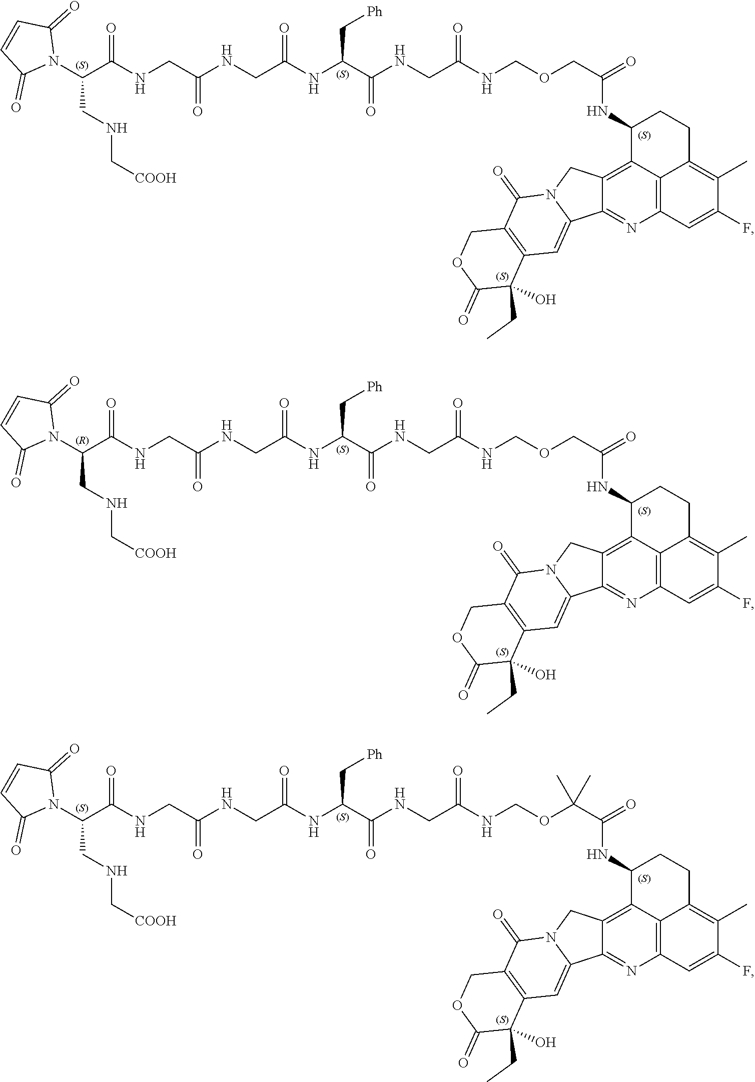

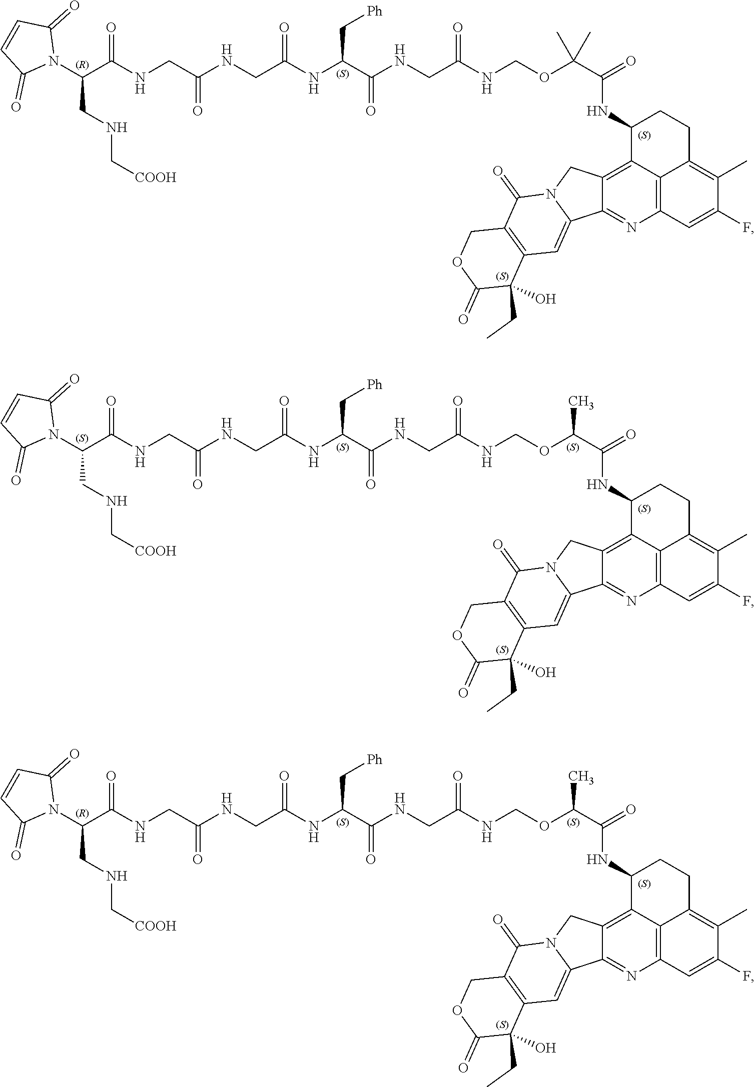

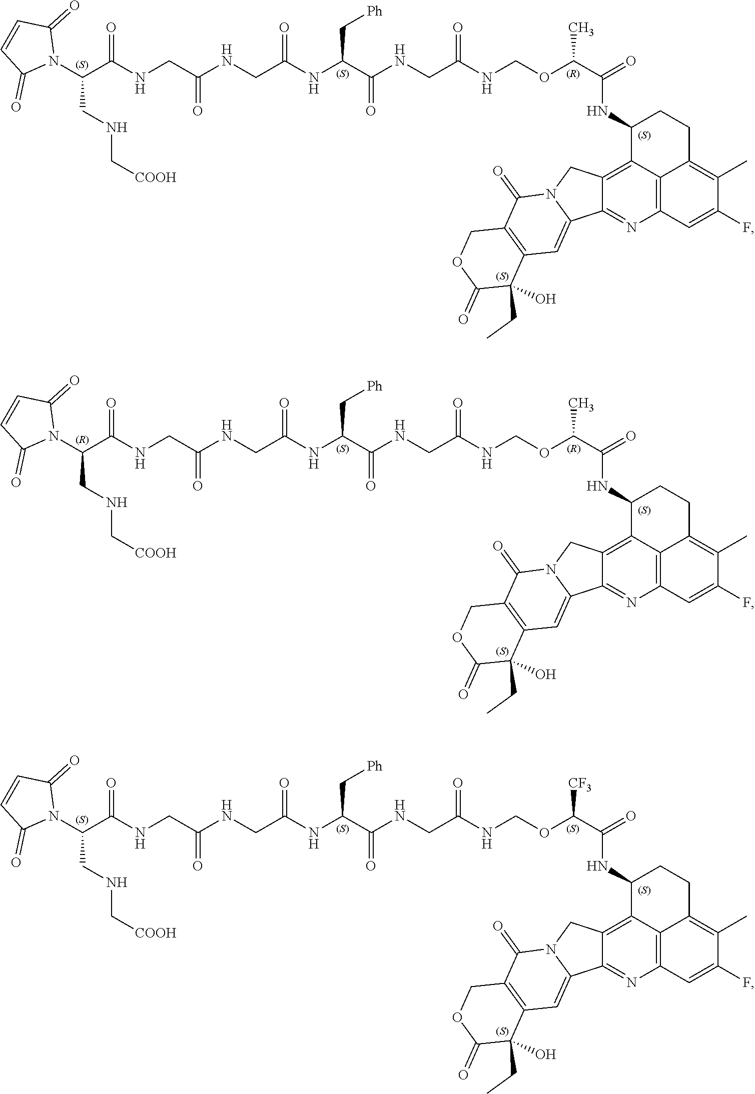

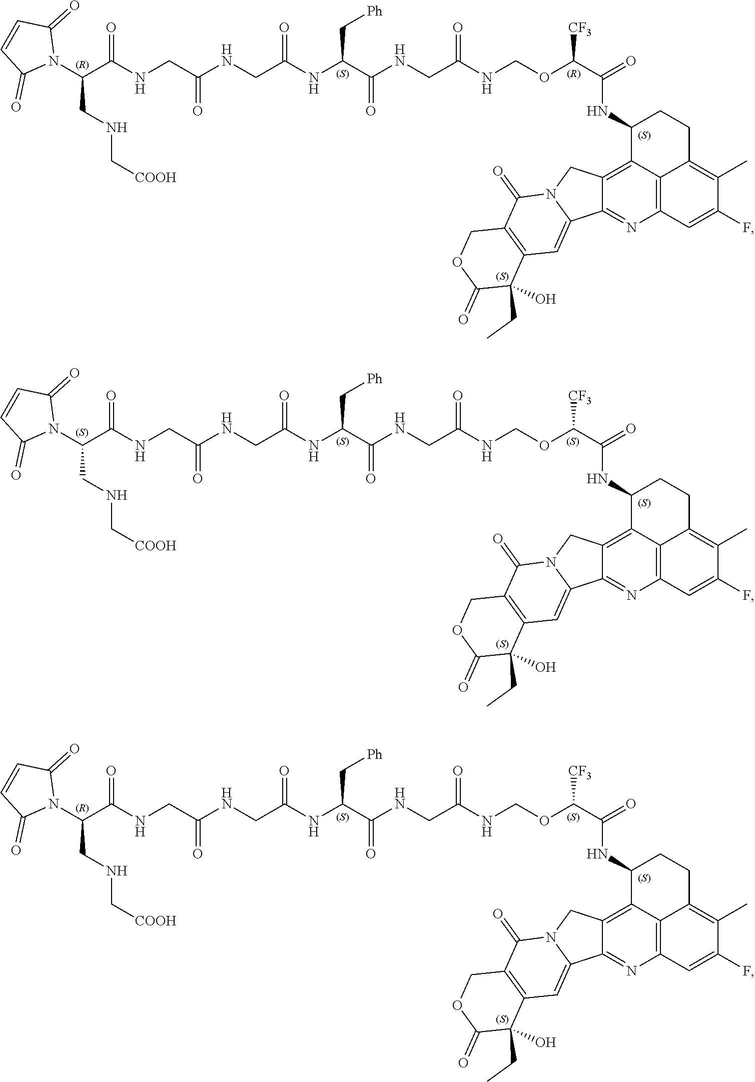

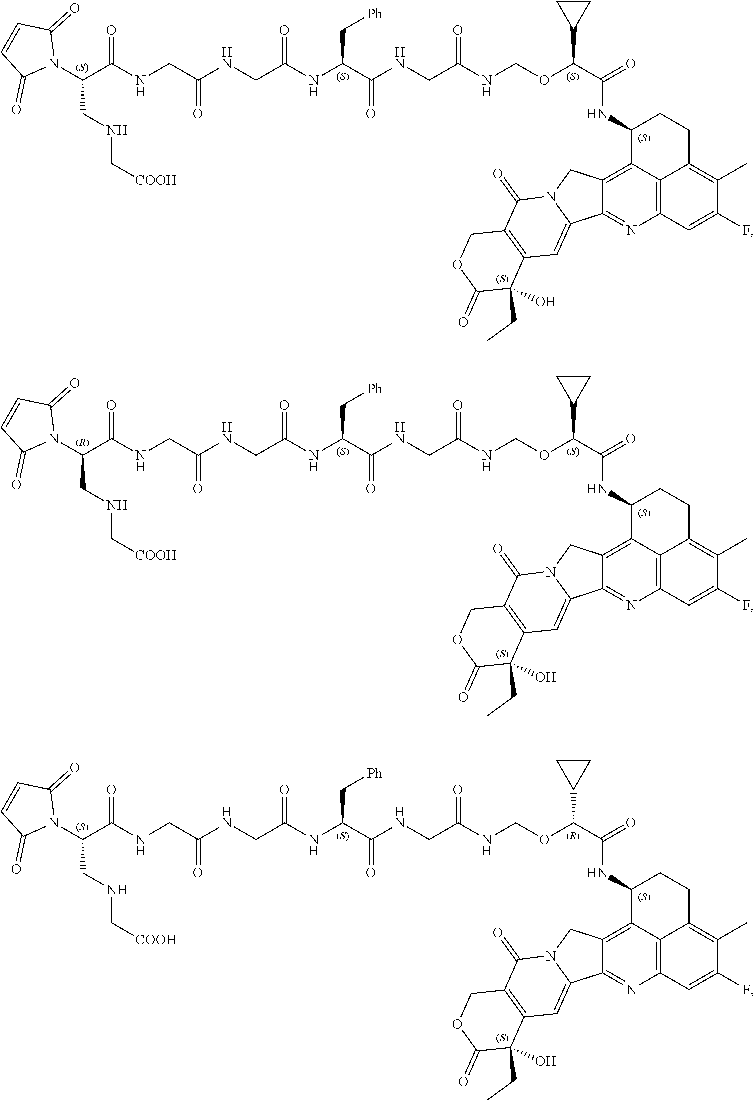

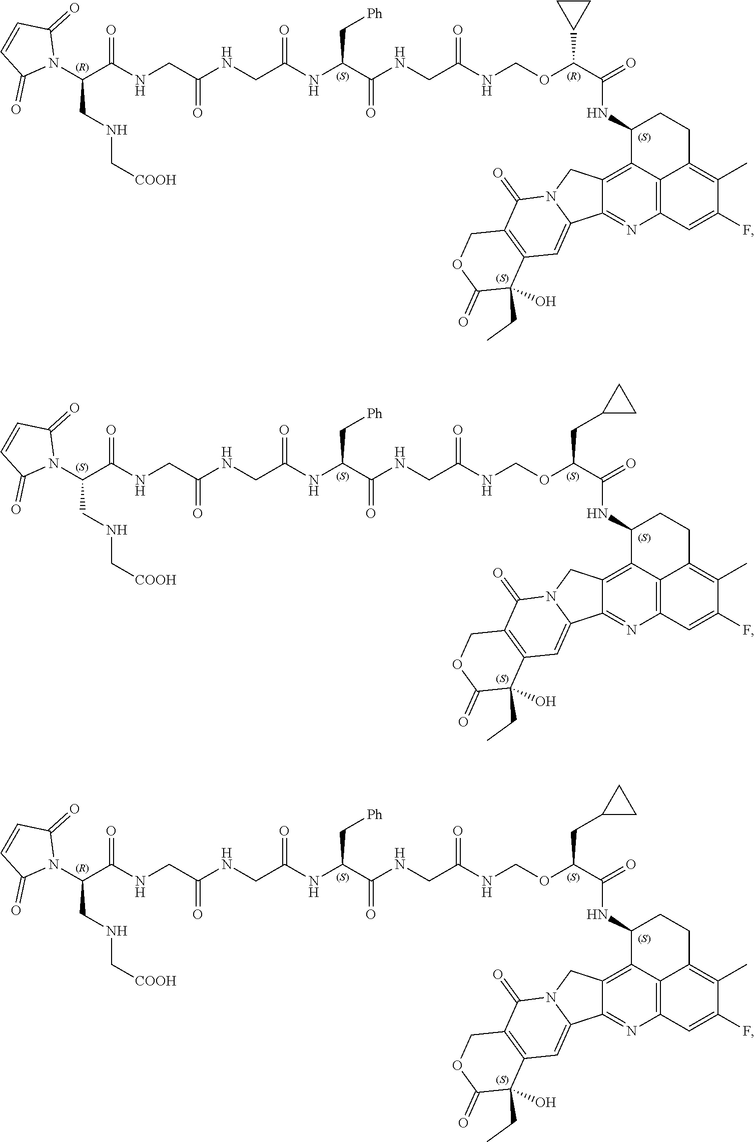

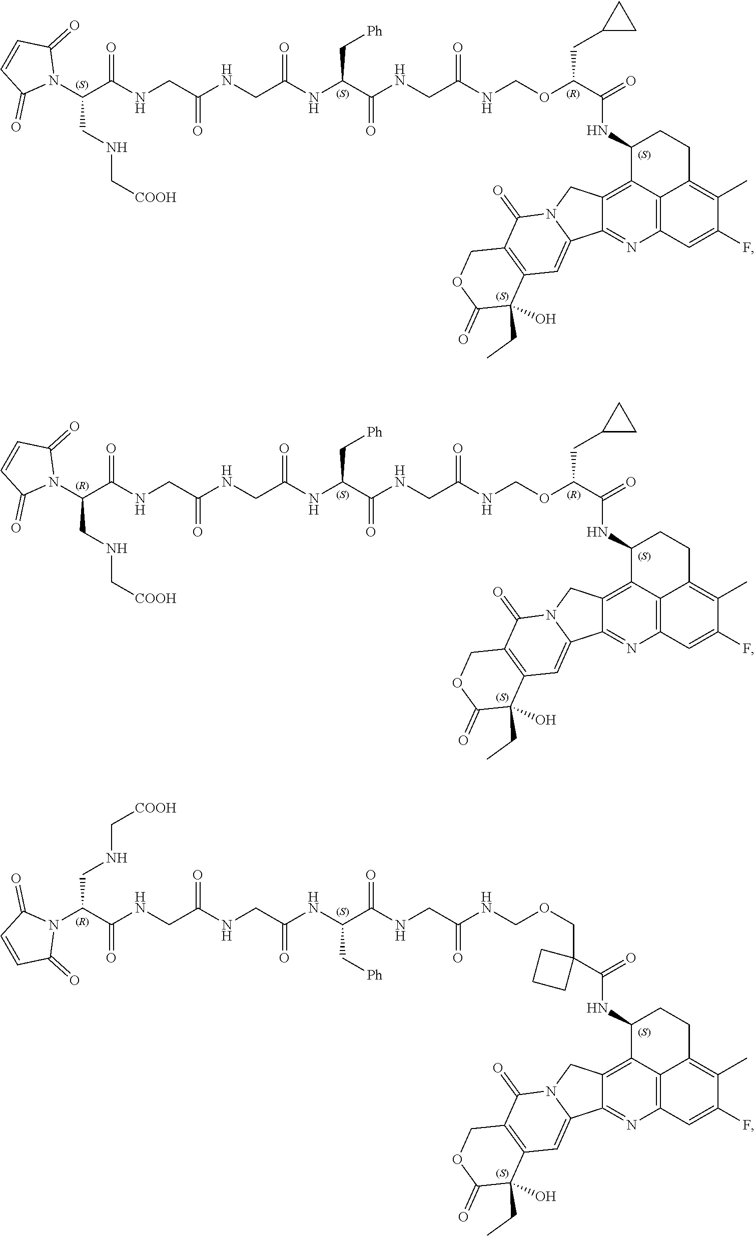

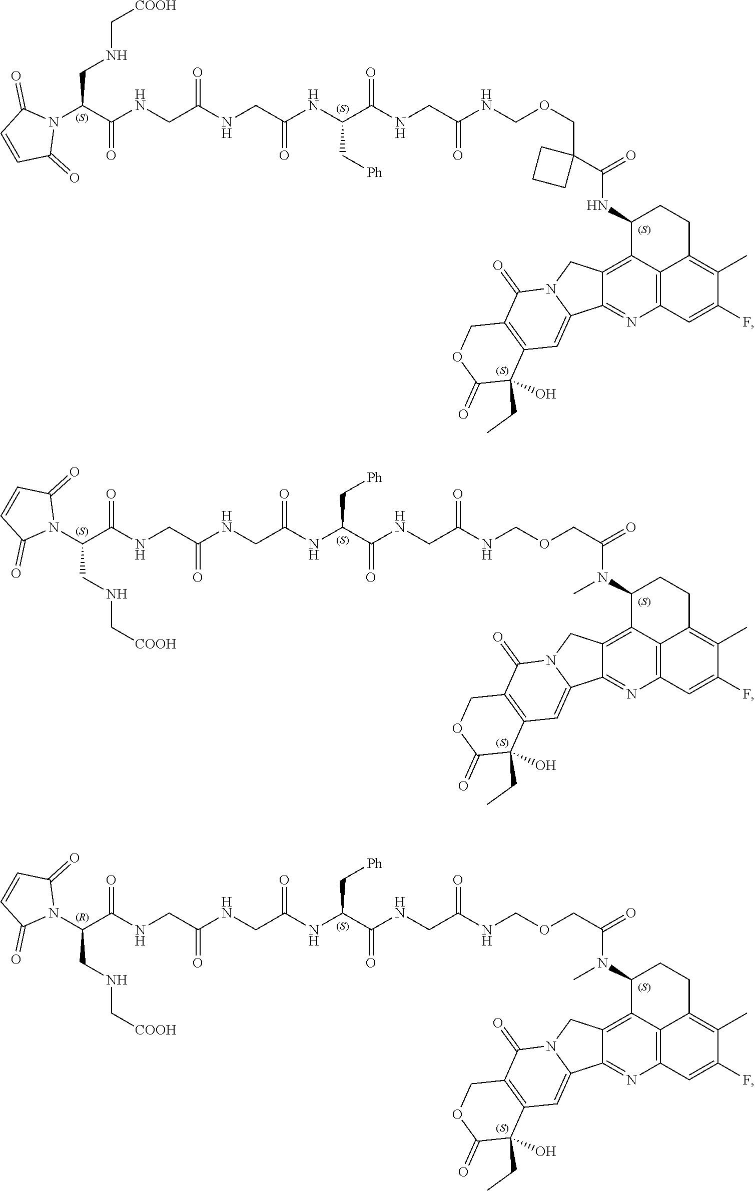

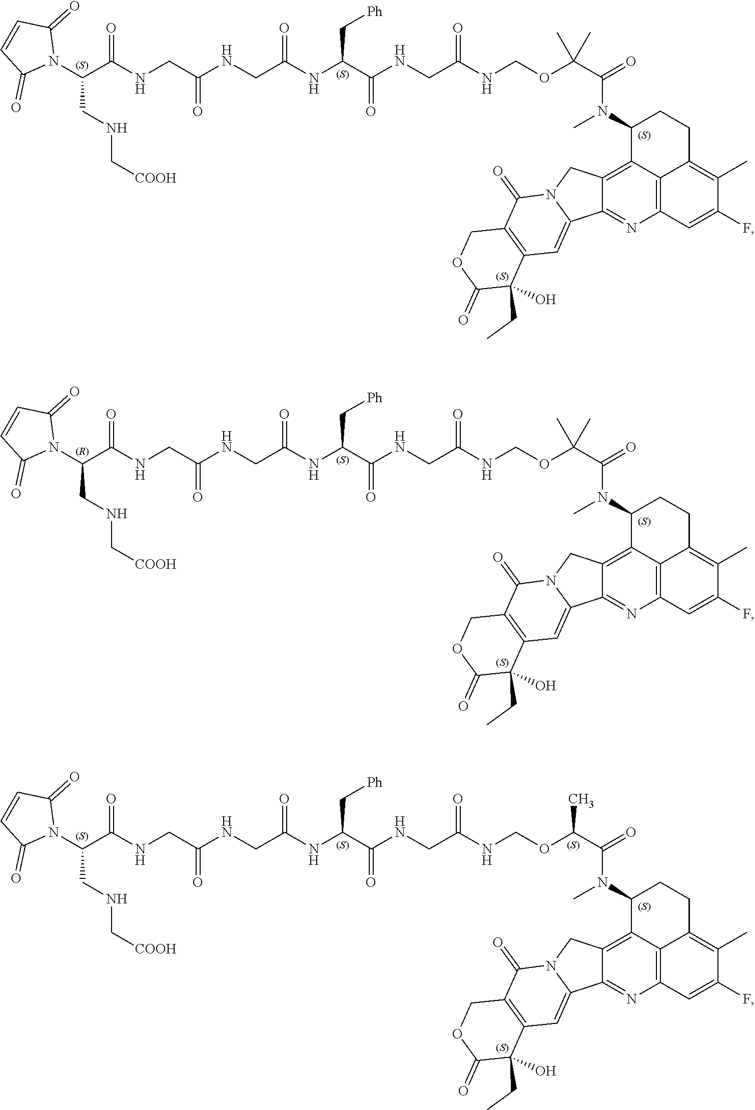

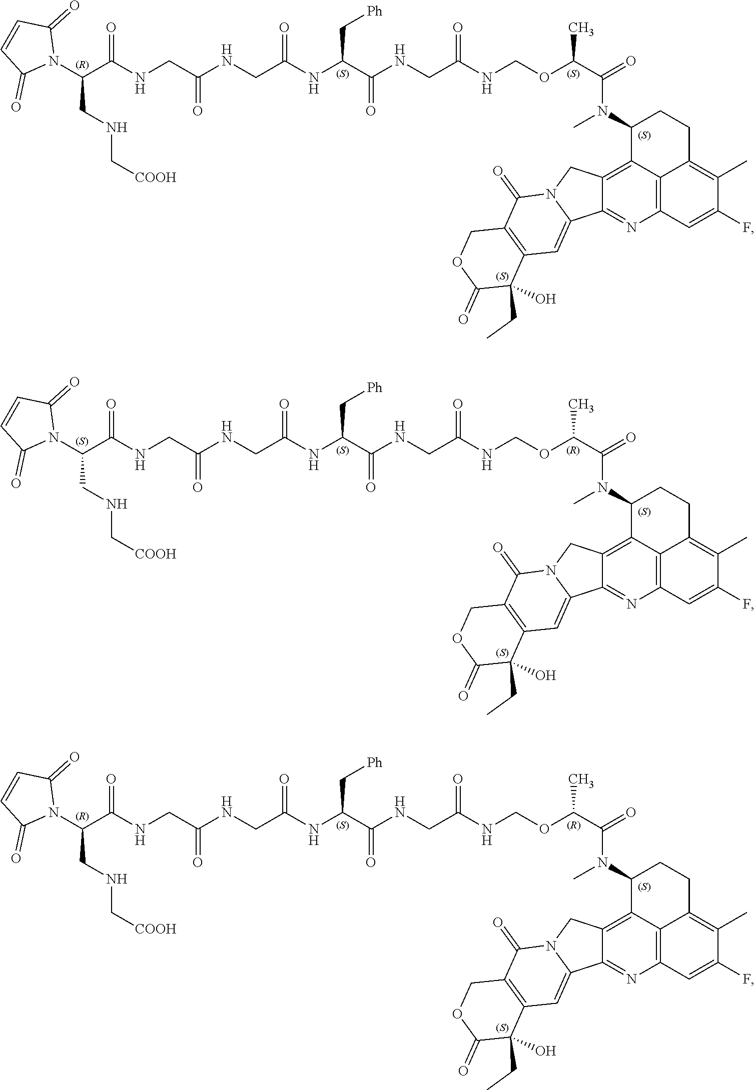

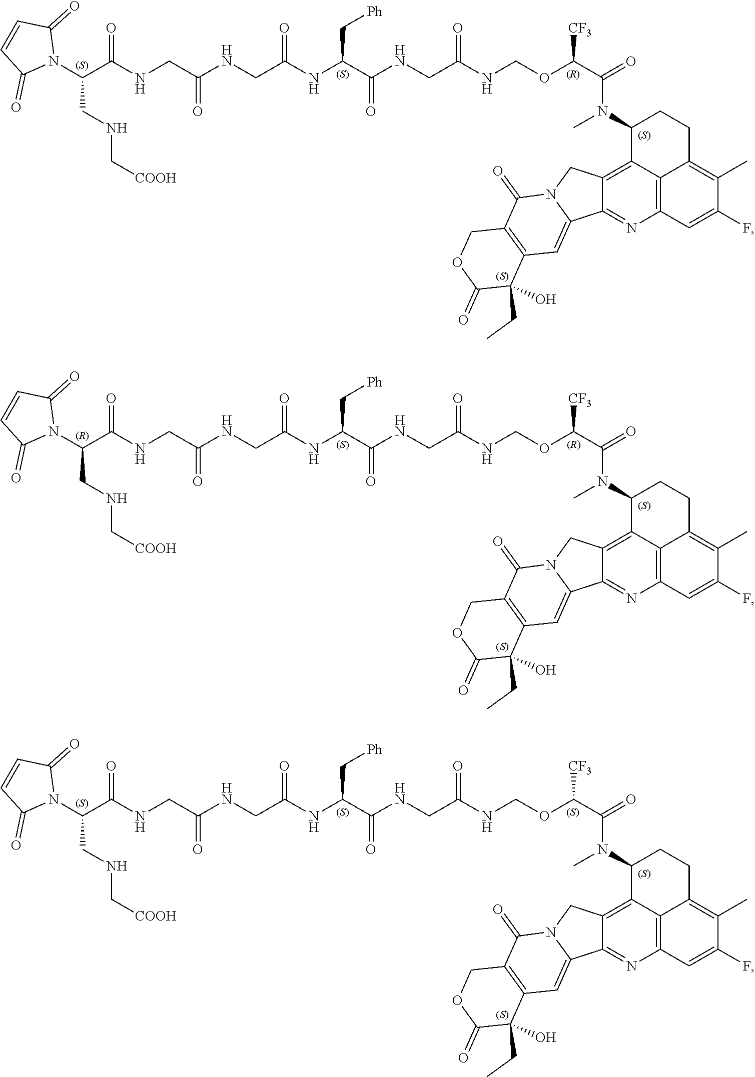

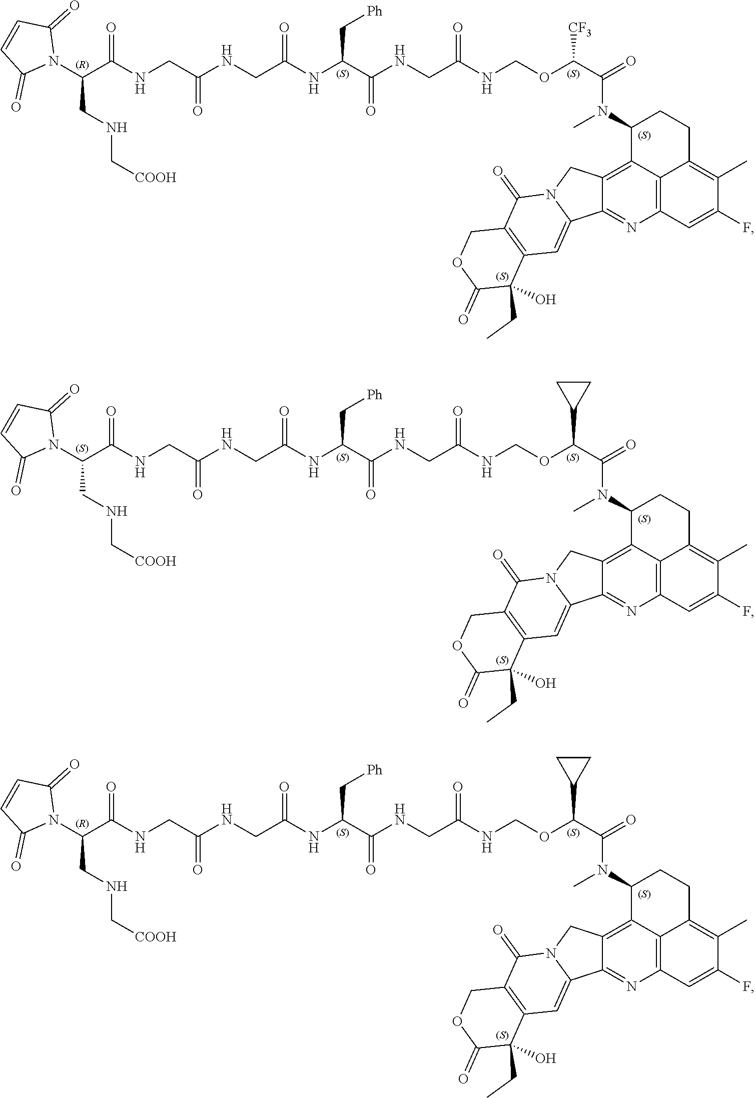

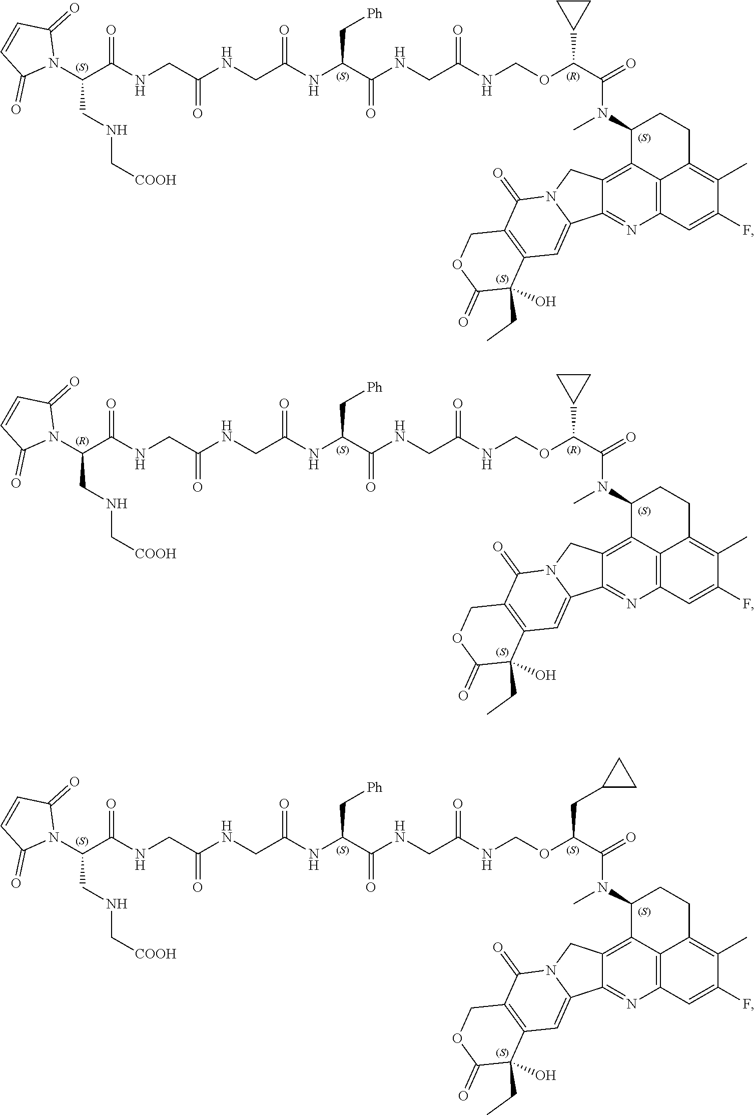

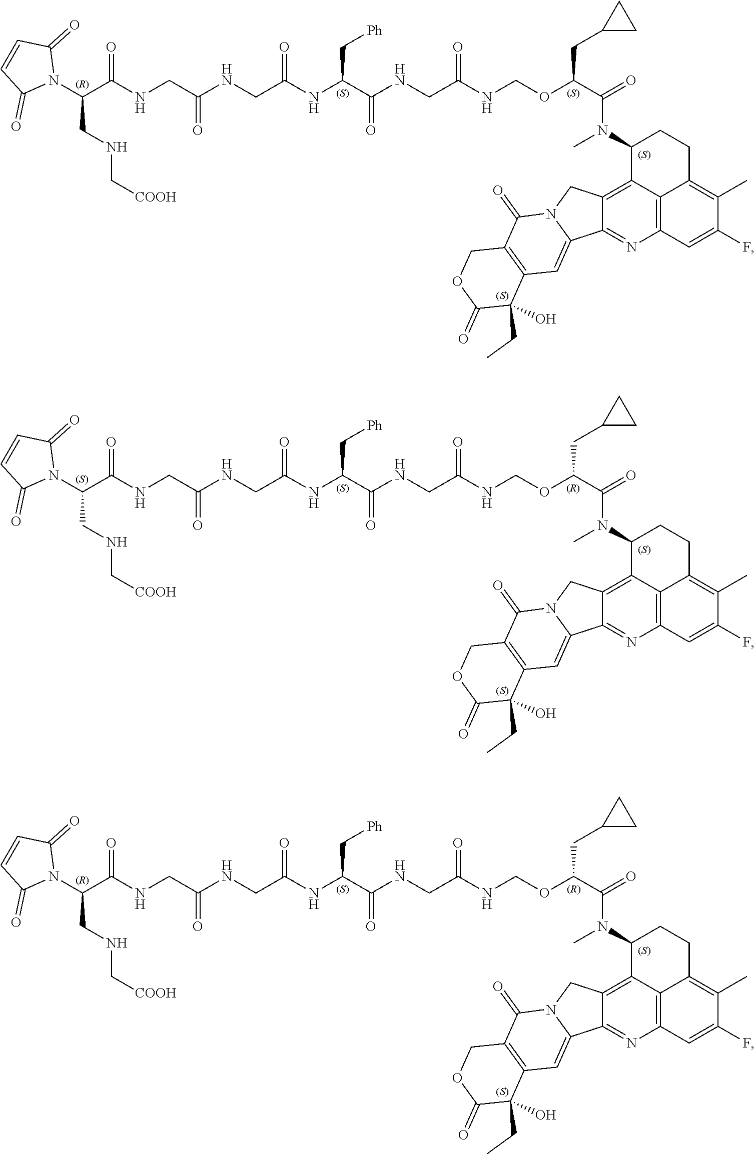

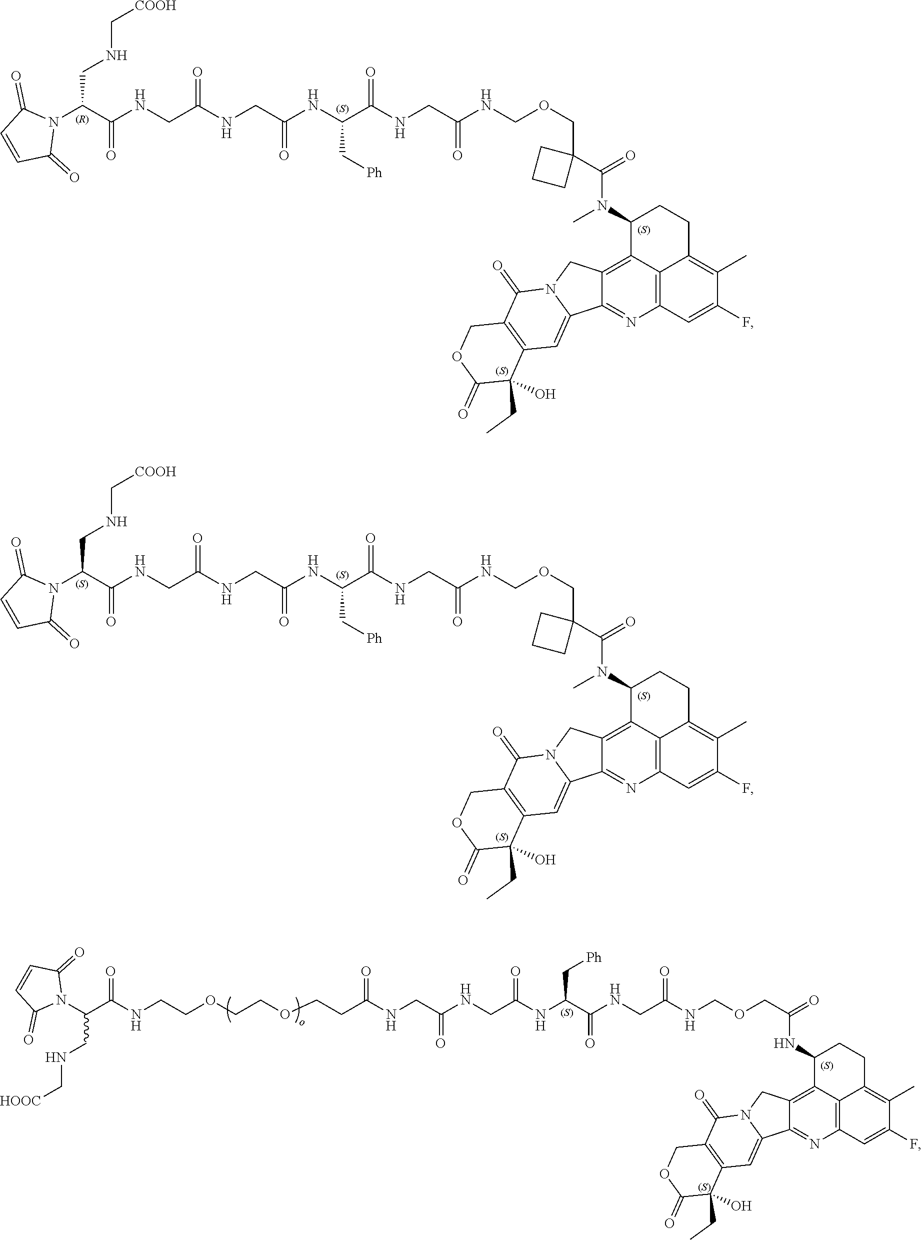

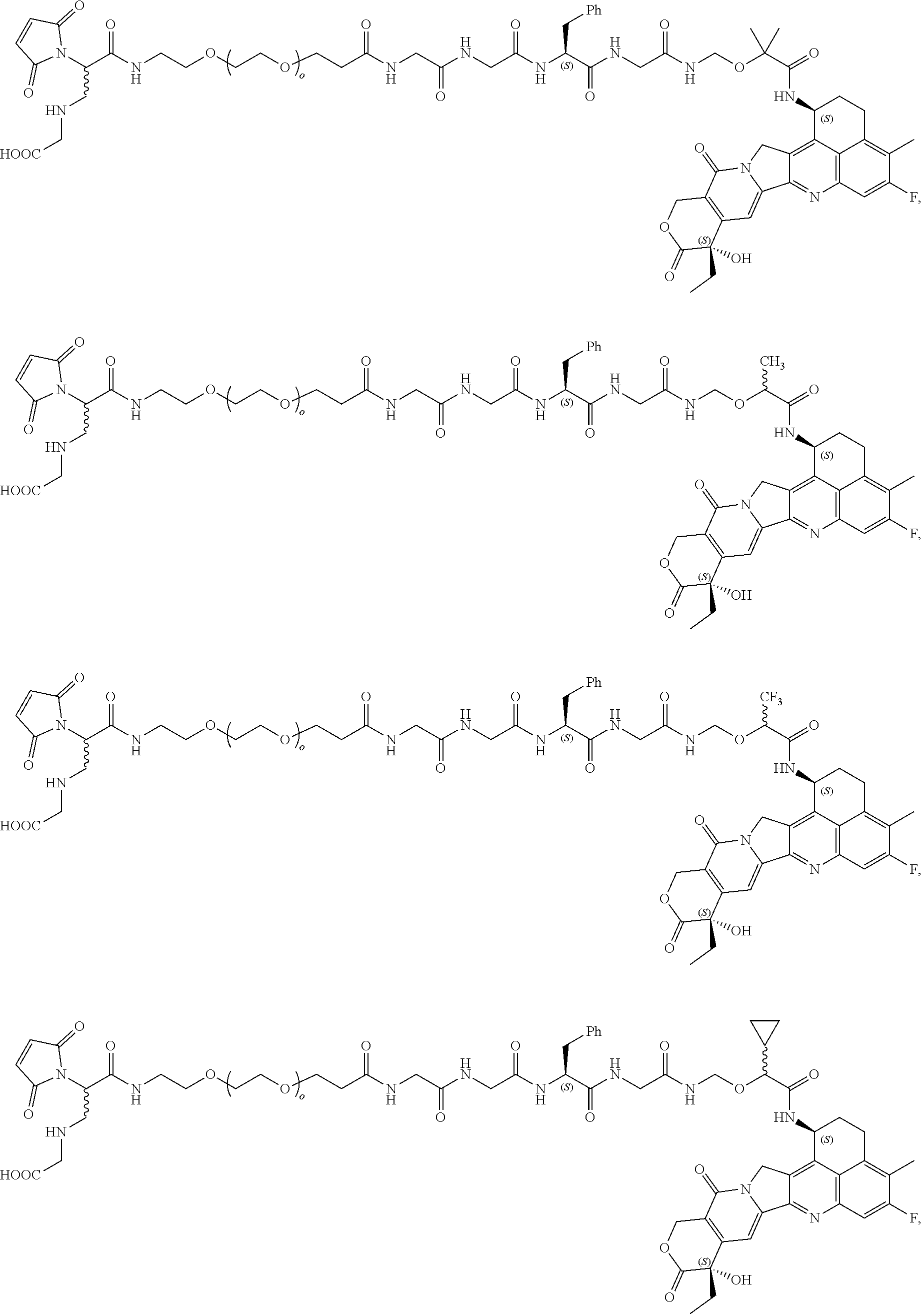

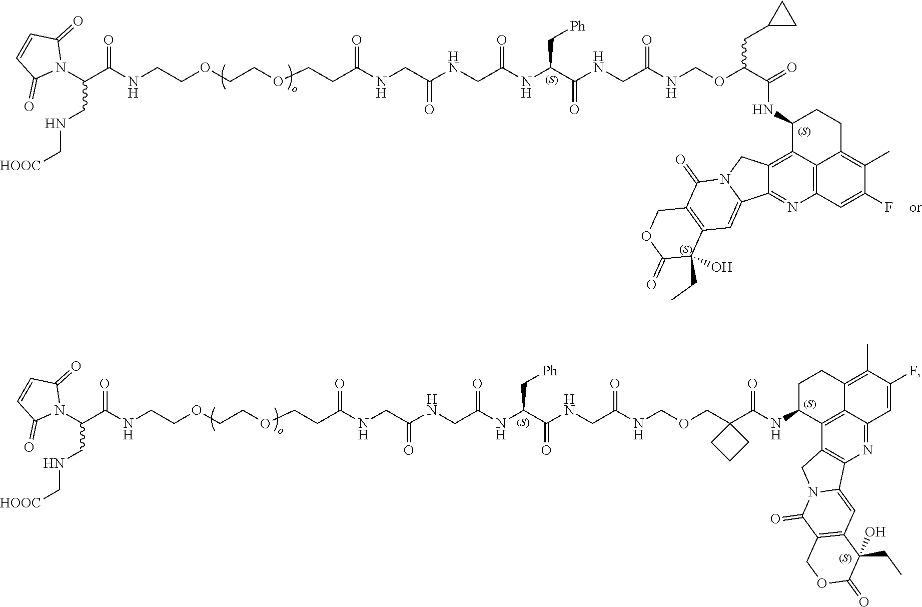

where: o is selected from the integers 1-10 (e.g., 1, 2, 3, 4, 5, 6, 7, 8, 9, or 10).

Said linker-drug compounds or pharmaceutically acceptable salts or solvates thereof disclosed in the third aspect of the present invention can be used as intermediates for coupling with the ligand Ab to form ligand-camptothecin derivative conjugates of formula I or formula II described in the first aspect and the second aspect.

A fourth aspect of the present invention discloses a method of preparing a ligand-camptothecin derivative conjugate or a pharmaceutically acceptable salt or solvate thereof as shown in general formula I or general formula II as described in the first and second aspects, characterized in that the method comprises the following steps,

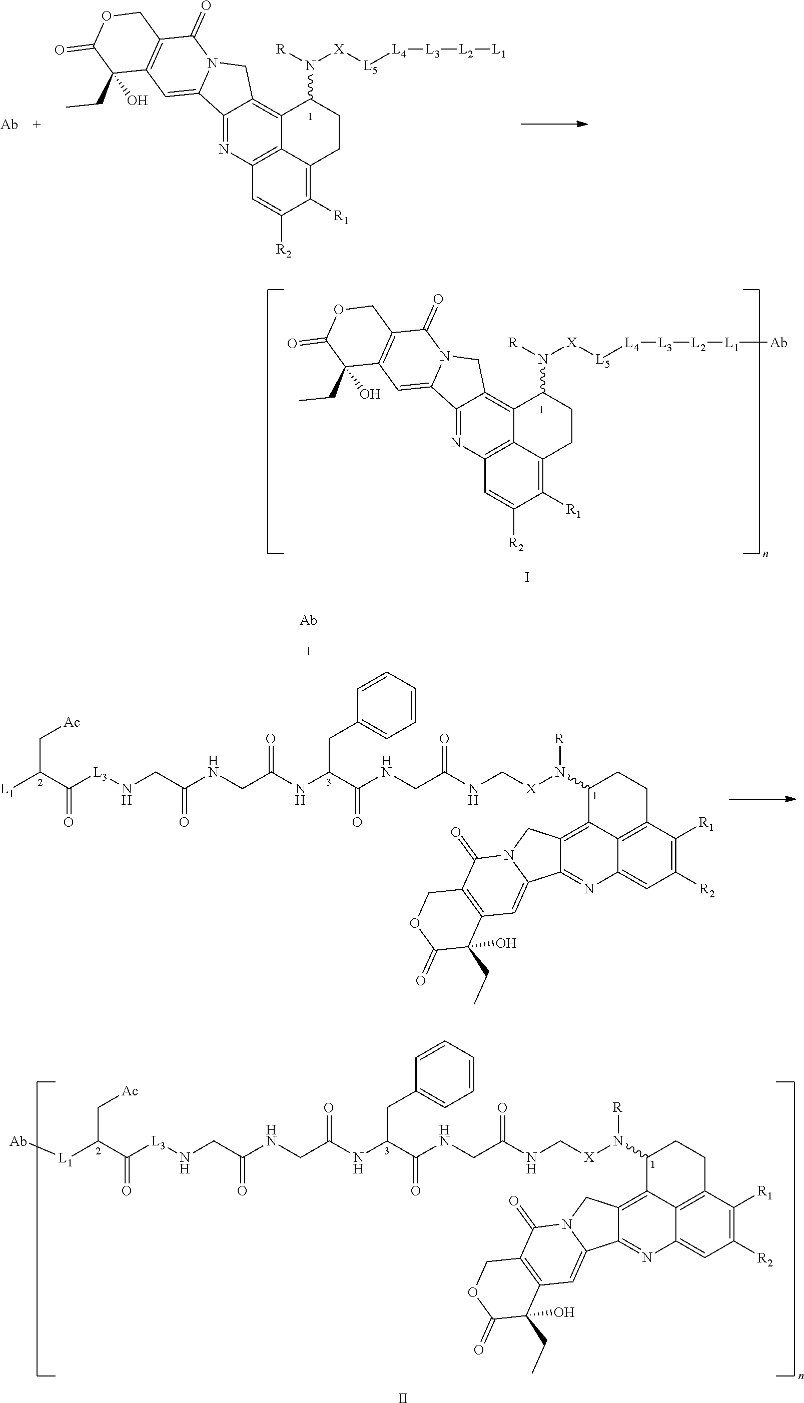

a ligand-camptothecin derivative conjugate as shown in general formula I or general formula II is obtained by a coupling reaction of a reduced antibody or antigen-binding fragment thereof with a linker-drug compound;
the chiral carbon atom at position 1, position 2, or position 3 has absolute chirality in the R configuration or S configuration;
Ab, L₁, L₂, L₃, L₄, L₅, X, R, R₁, R₂ and n are as previously described.

The present application also relates to the use of the linker-drug compounds or pharmaceutically acceptable salts or solvates thereof, disclosed and described in the third aspect, as intermediates in the preparation of ligand-camptothecin derivative conjugates or pharmaceutically acceptable salts or solvates thereof. In certain embodiments, said ligand-camptothecin derivative conjugates or pharmaceutically acceptable salts or solvates thereof are the ligand-camptothecin derivative conjugates or pharmaceutically acceptable salts or solvates thereof disclosed in the first, second and fourth aspects of the present invention. In certain embodiments, said preparation is carried out according to the method of preparation disclosed in the fourth aspect.

In some embodiments of the first aspect, the second aspect and the fourth aspect of the present invention, the disclosure of said ligand-camptothecin derivative conjugate, or a pharmaceutically acceptable salt or solvate thereof, is characterized in that said ligand-camptothecin derivative conjugate, or a pharmaceutically acceptable salt or solvate thereof, is selected without limitation from the following structures, or succinimide open-ring structures thereof, or isomers thereof,

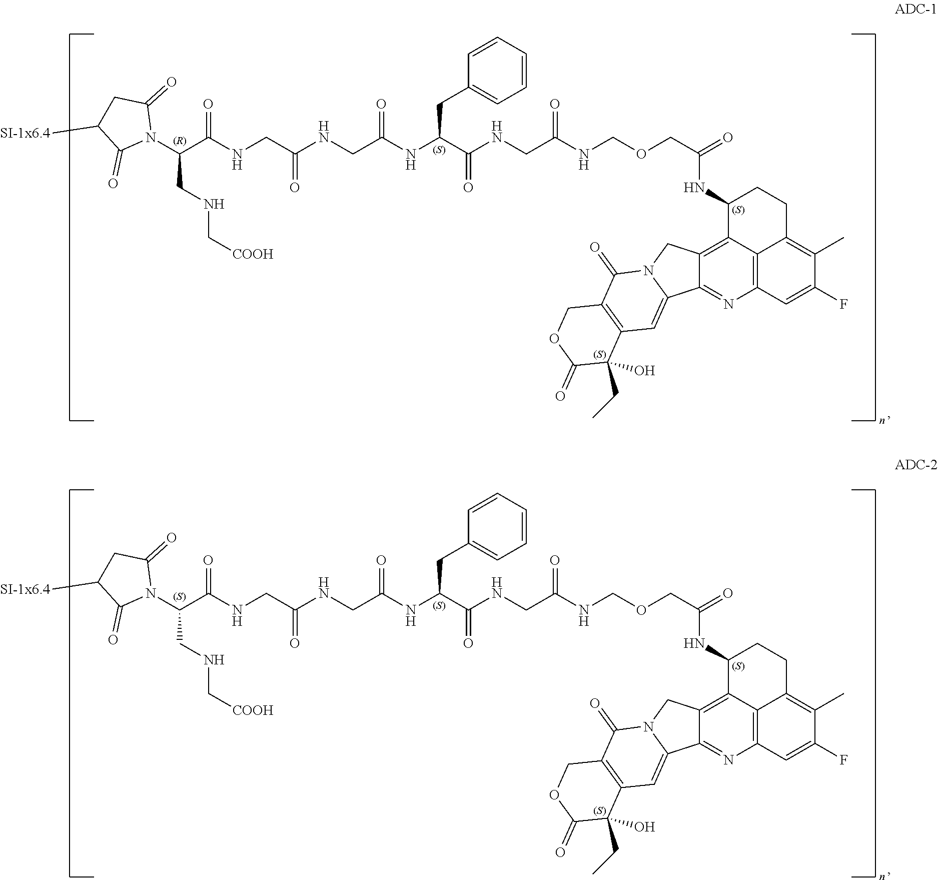

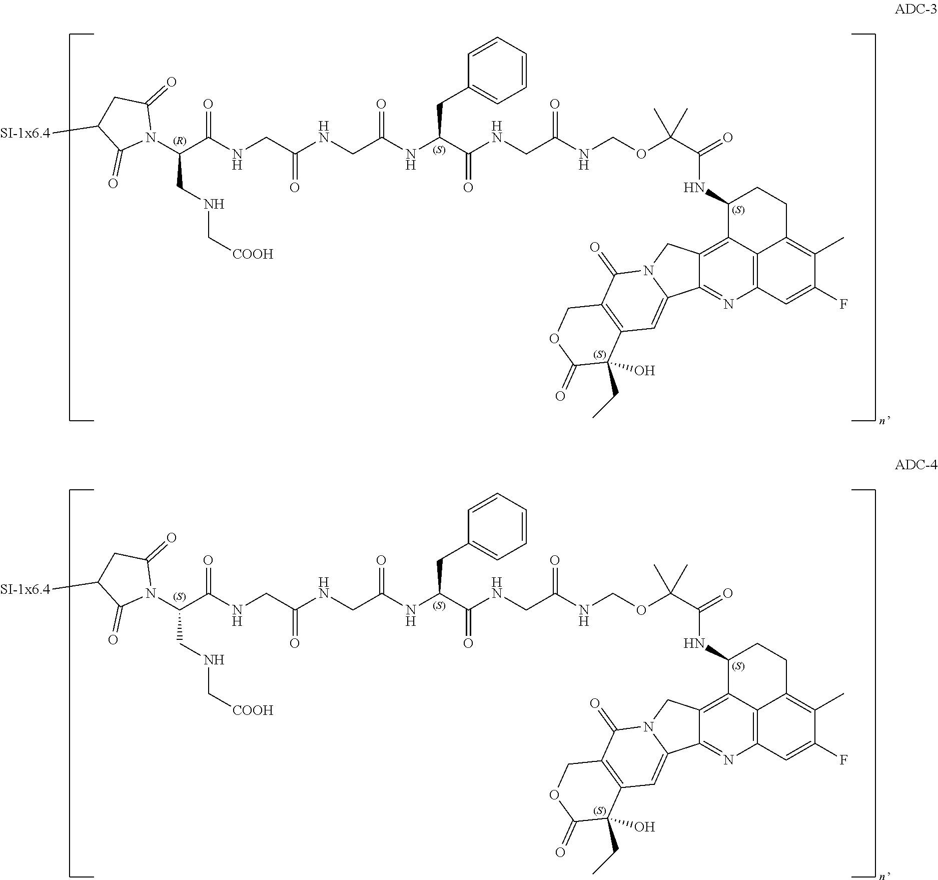

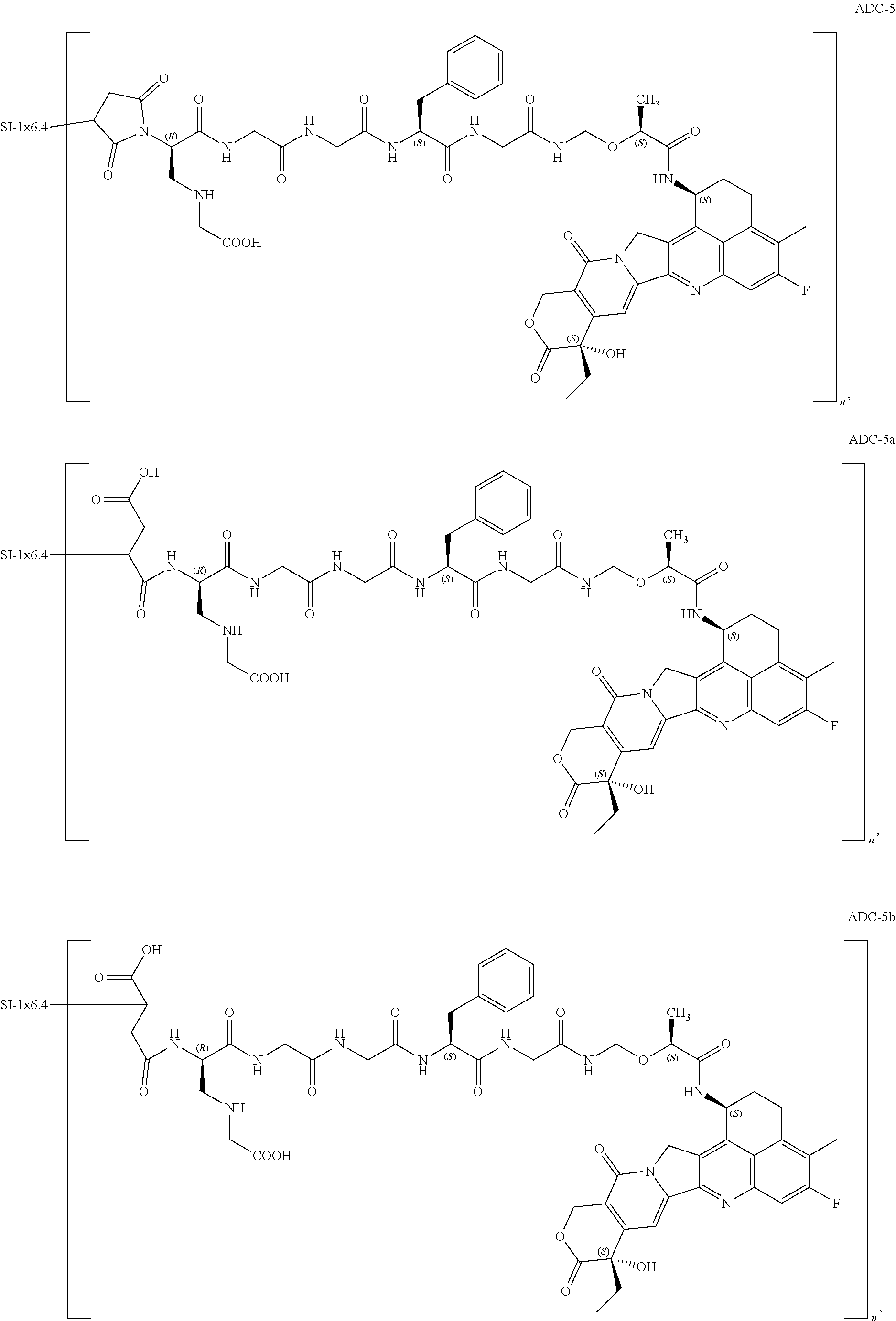

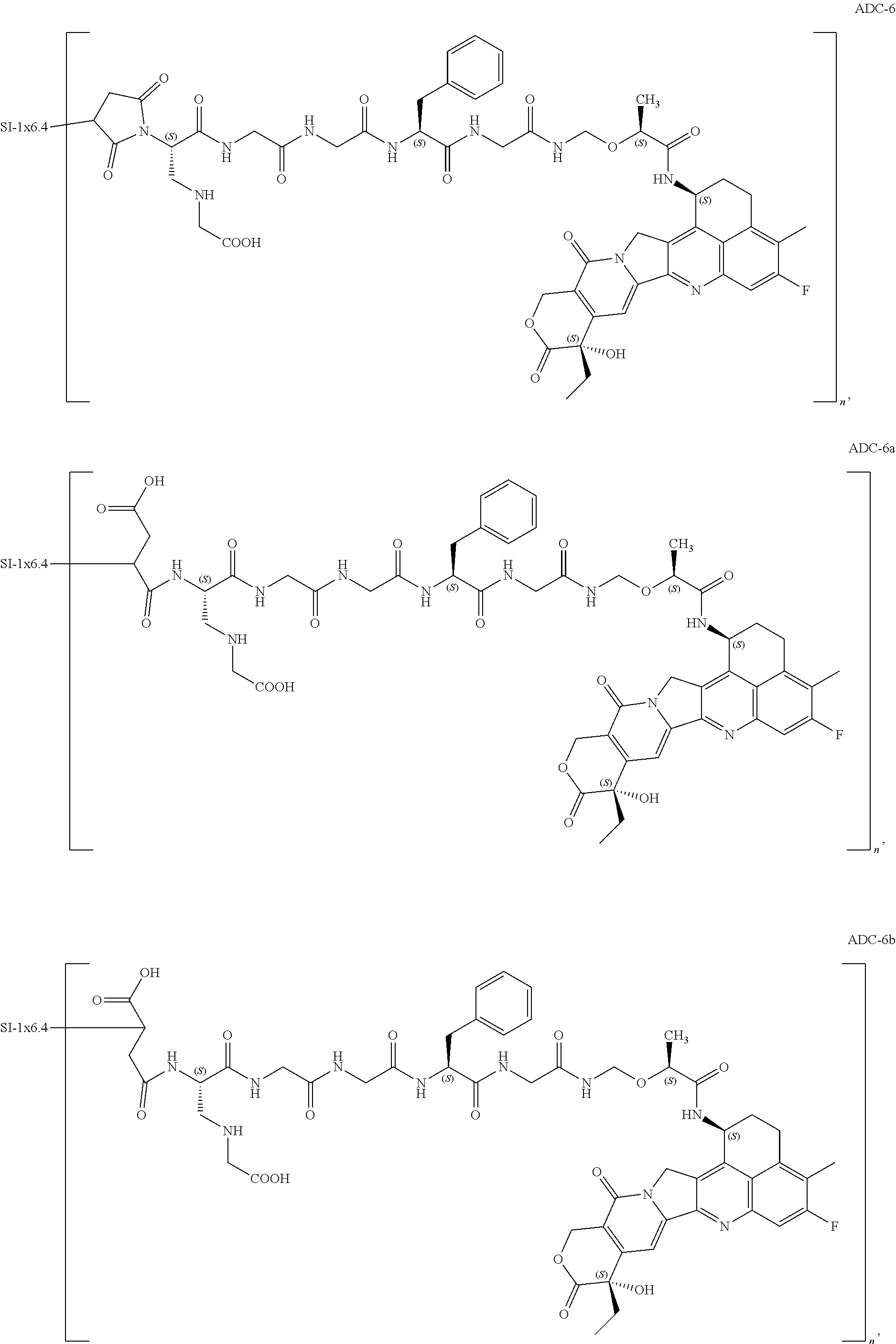

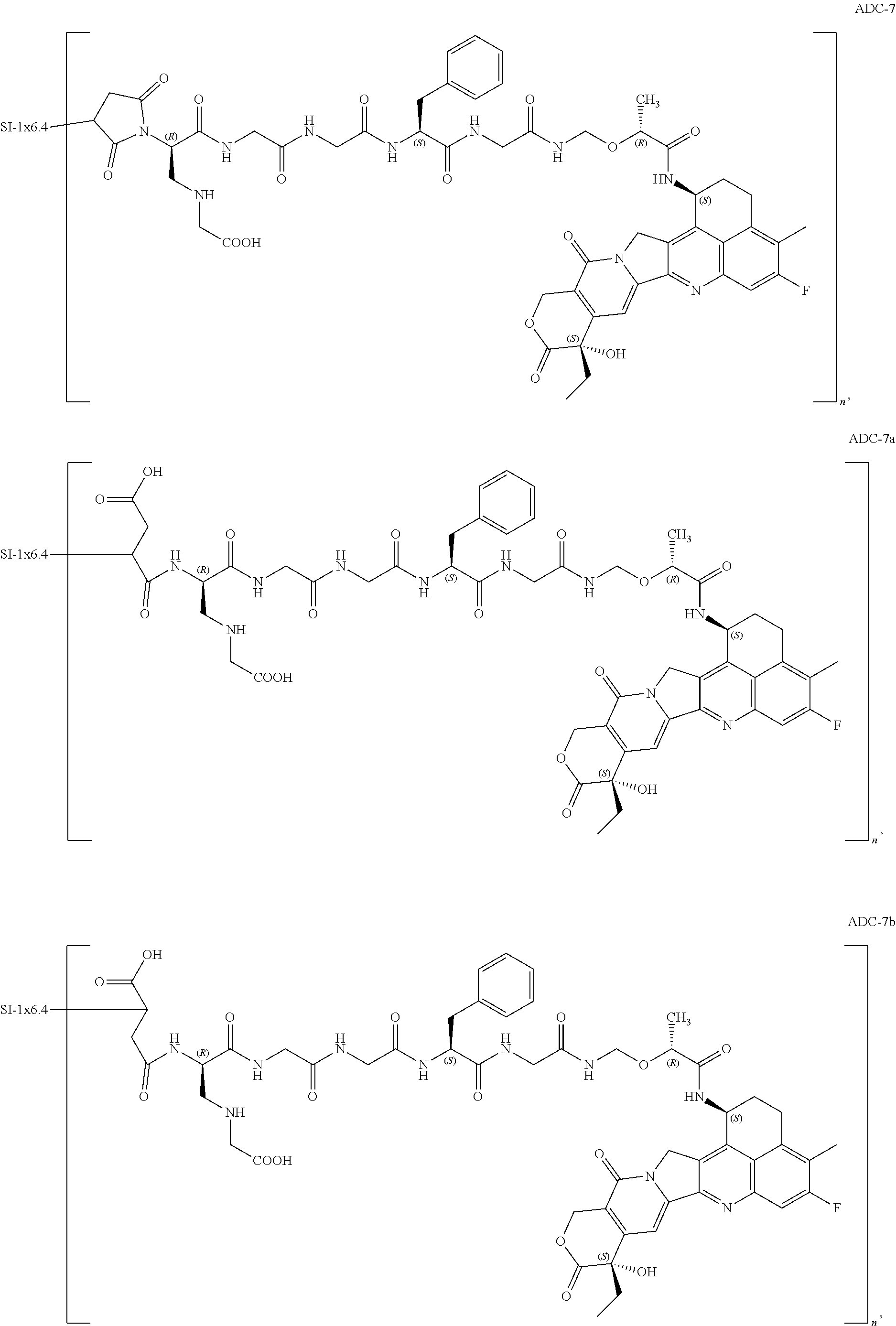

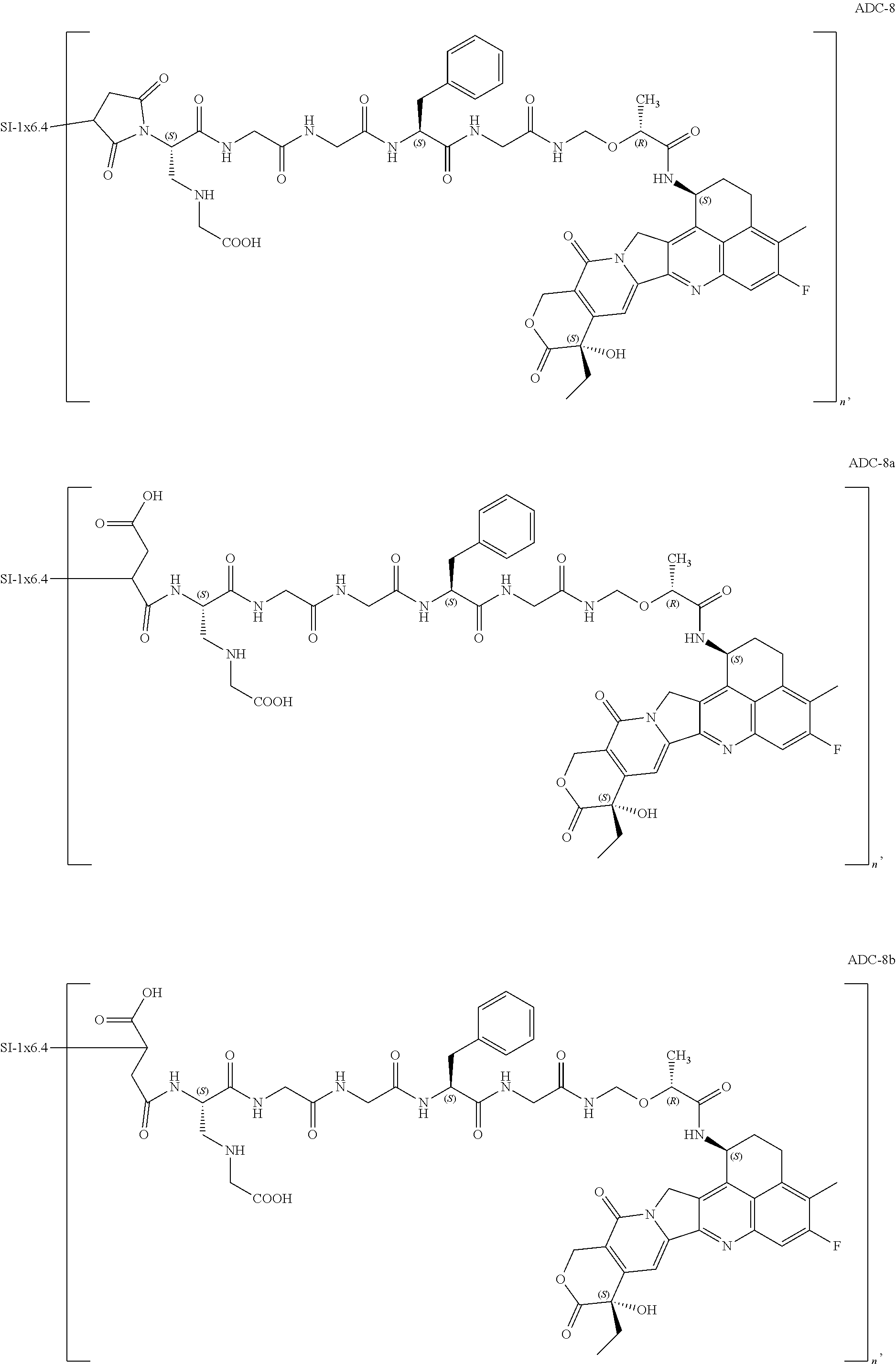

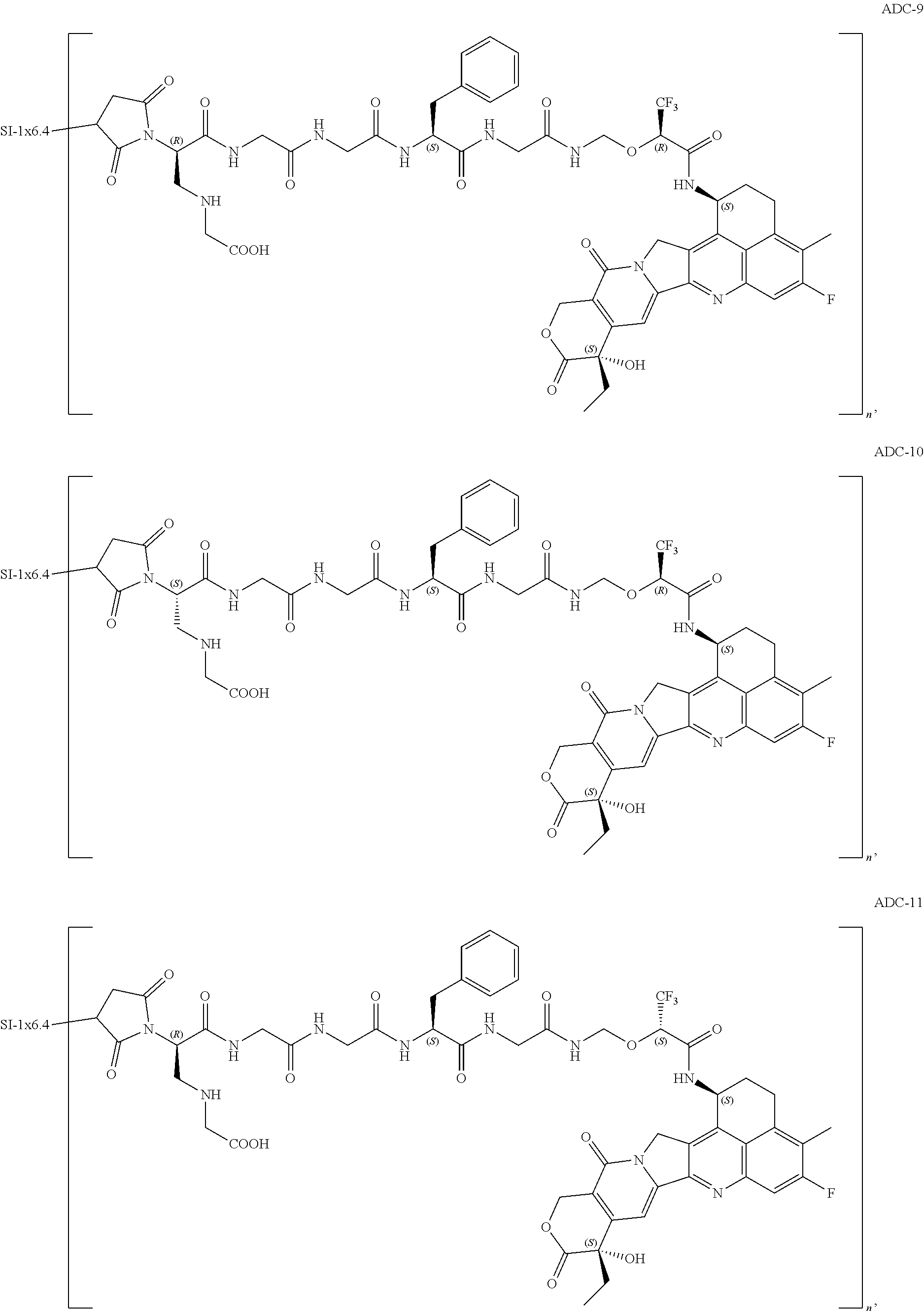

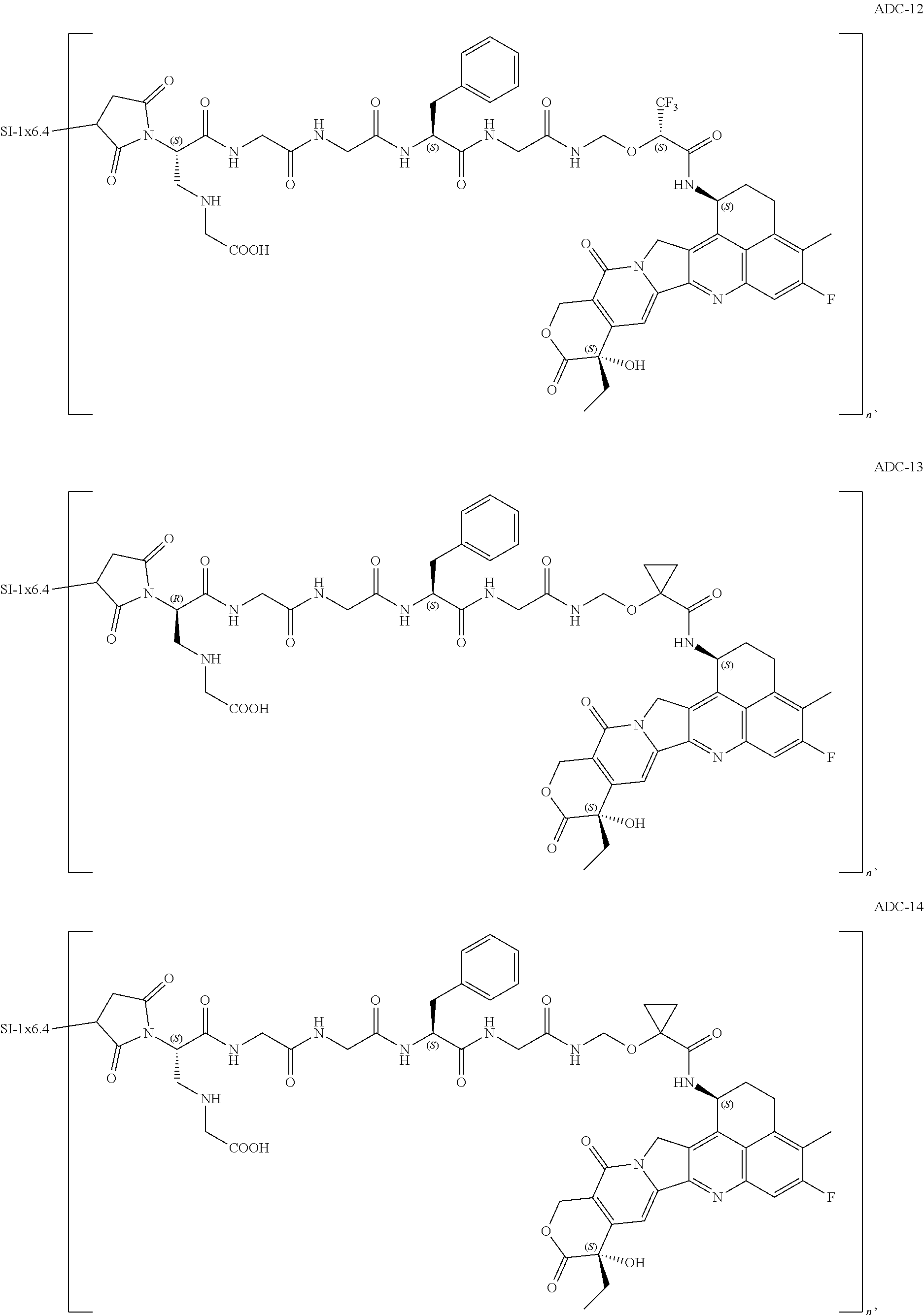

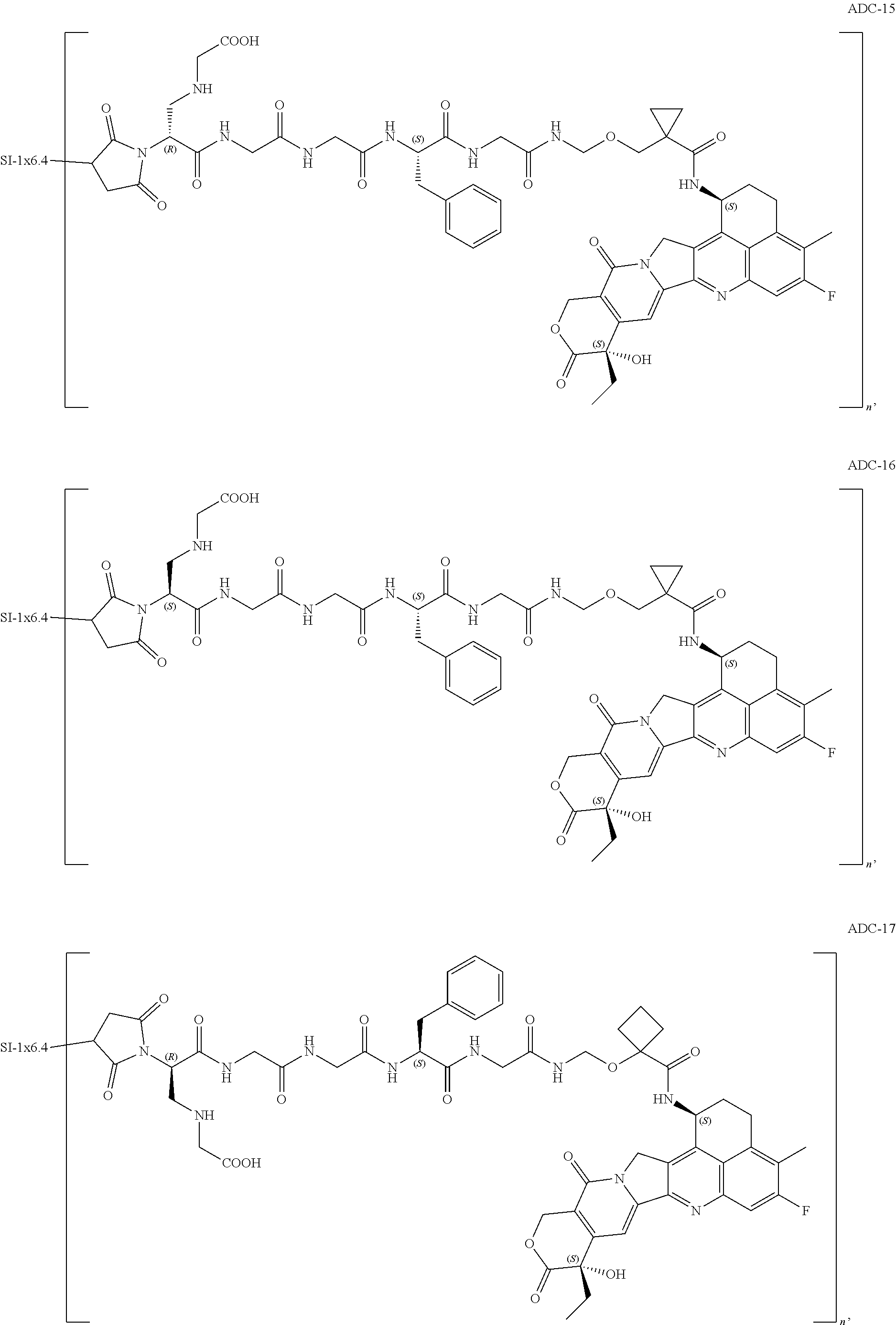

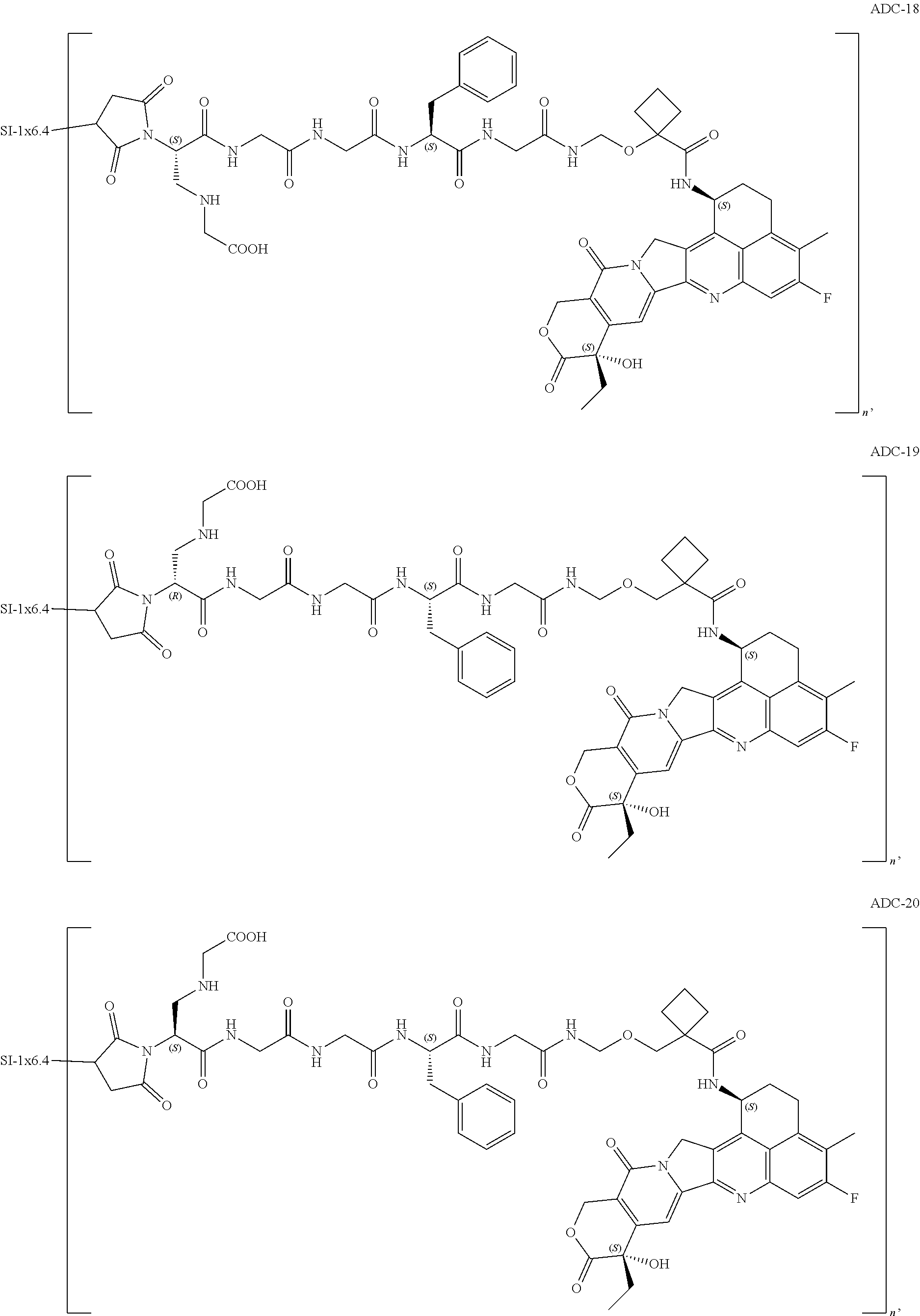

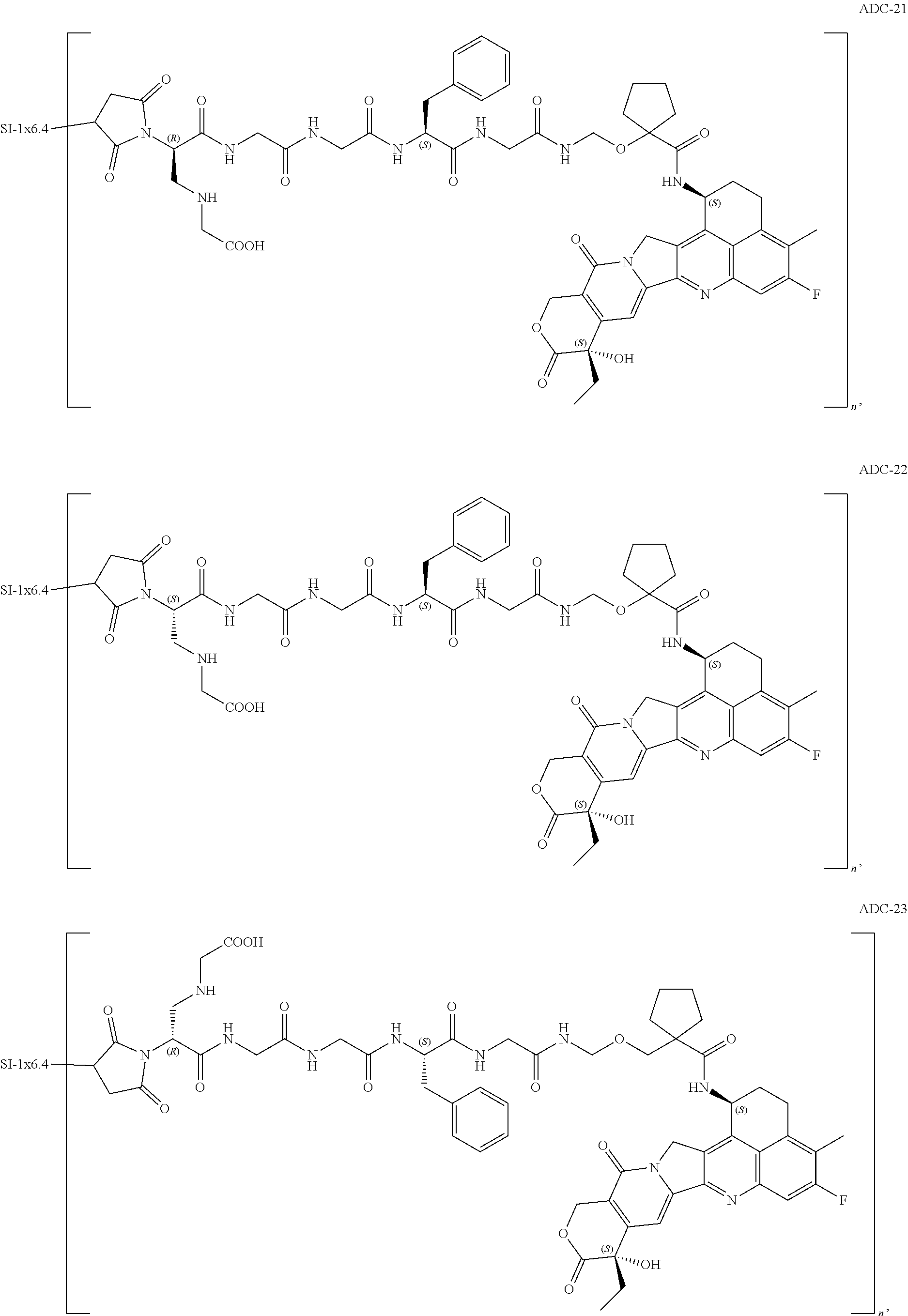

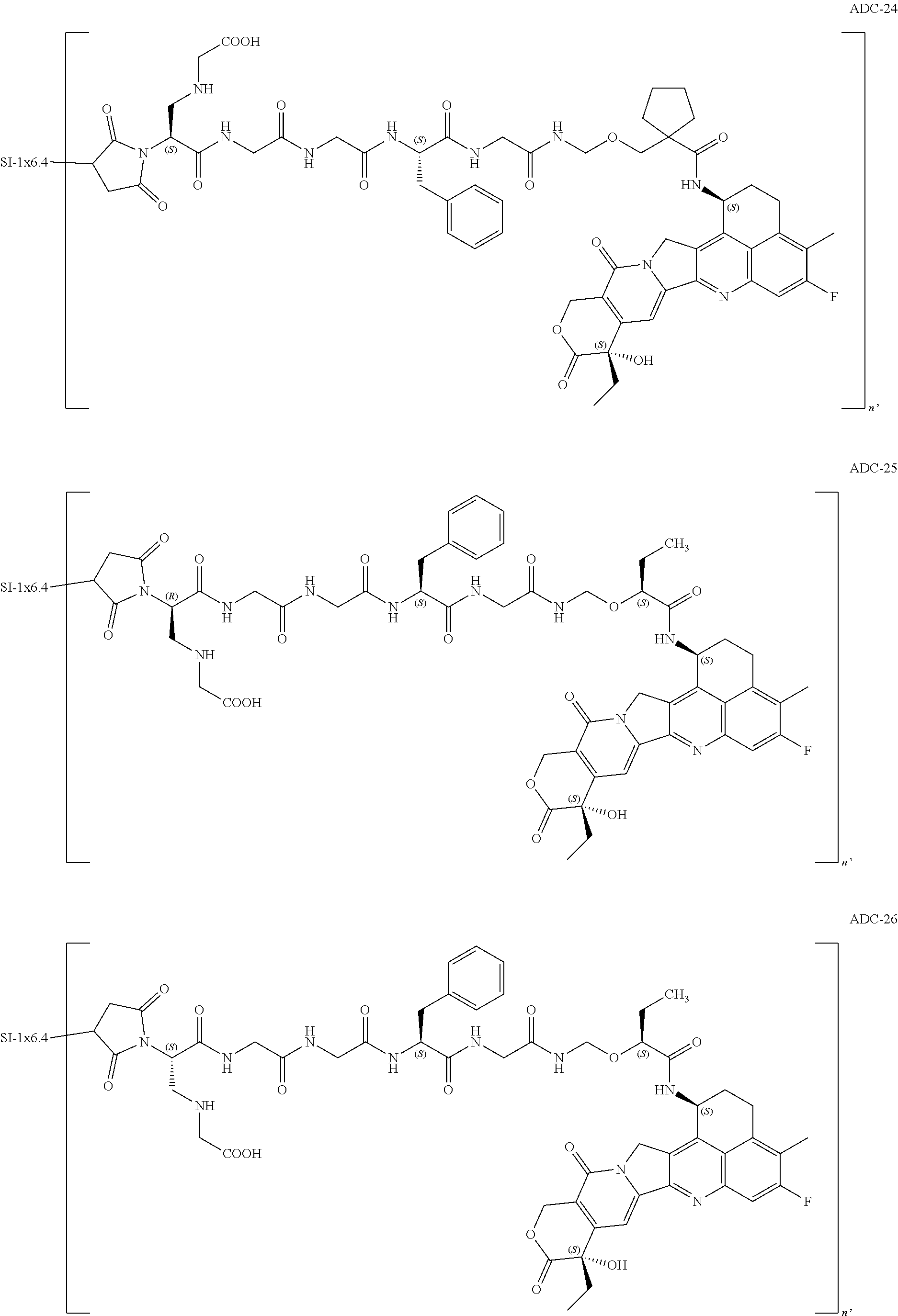

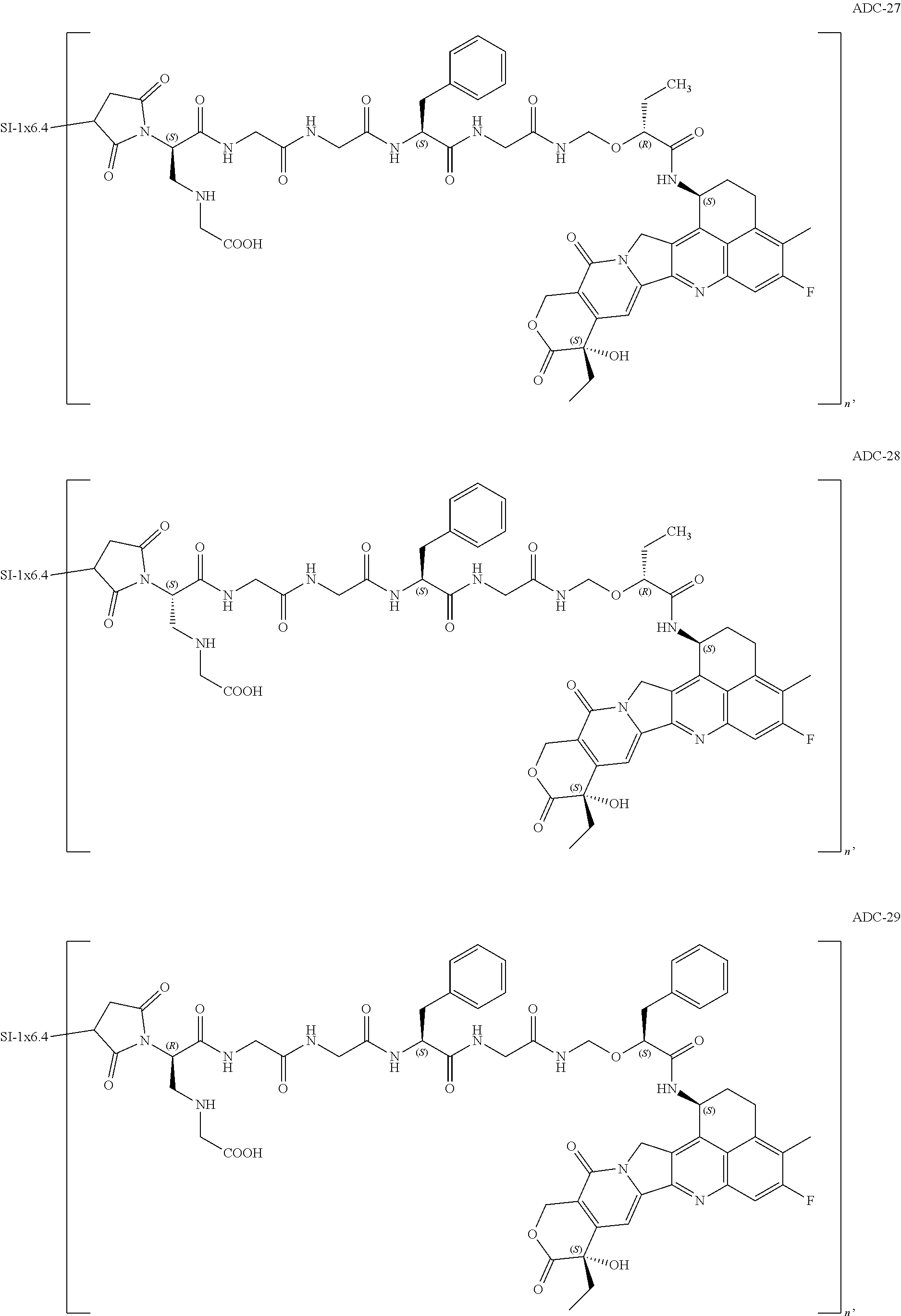

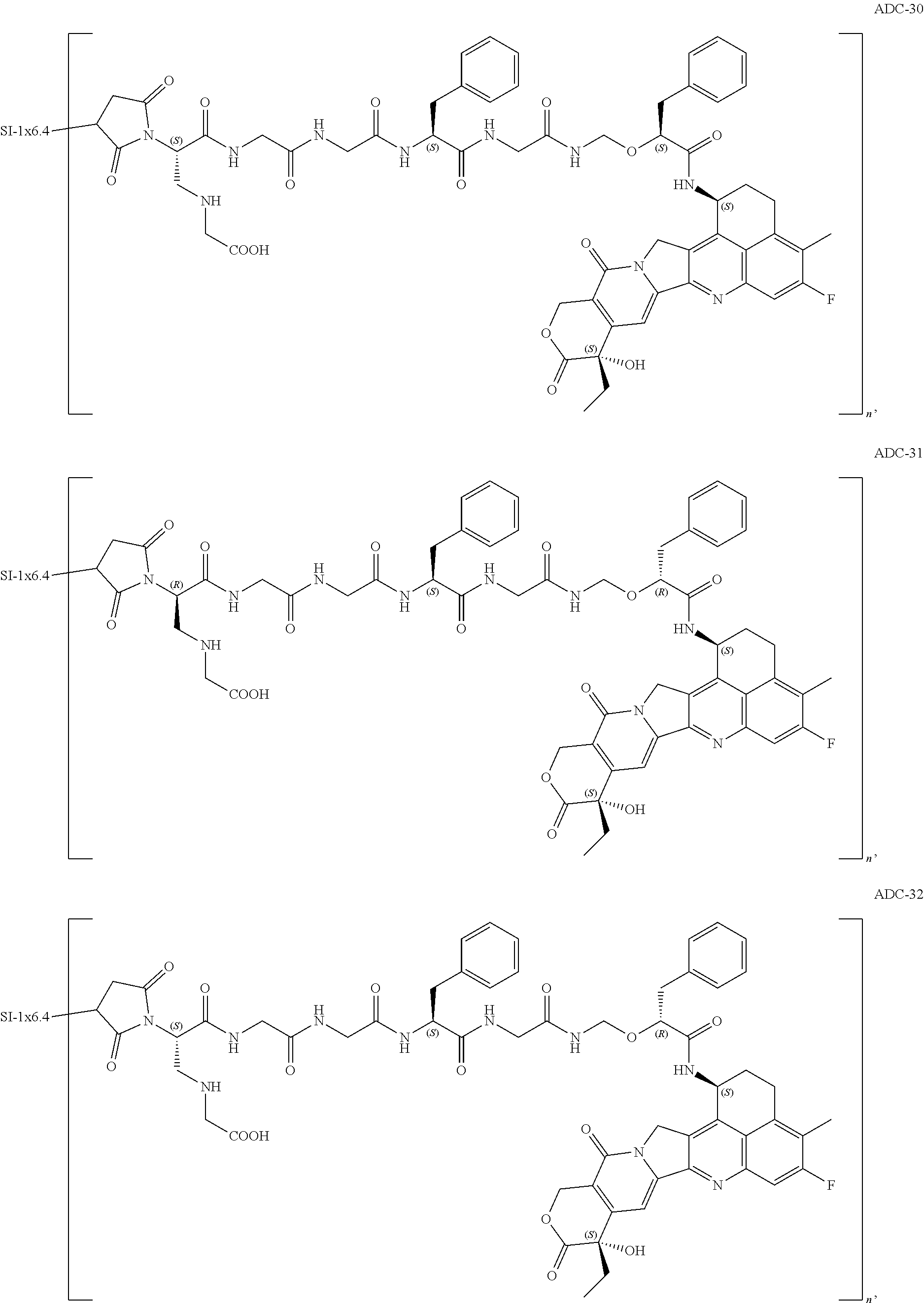

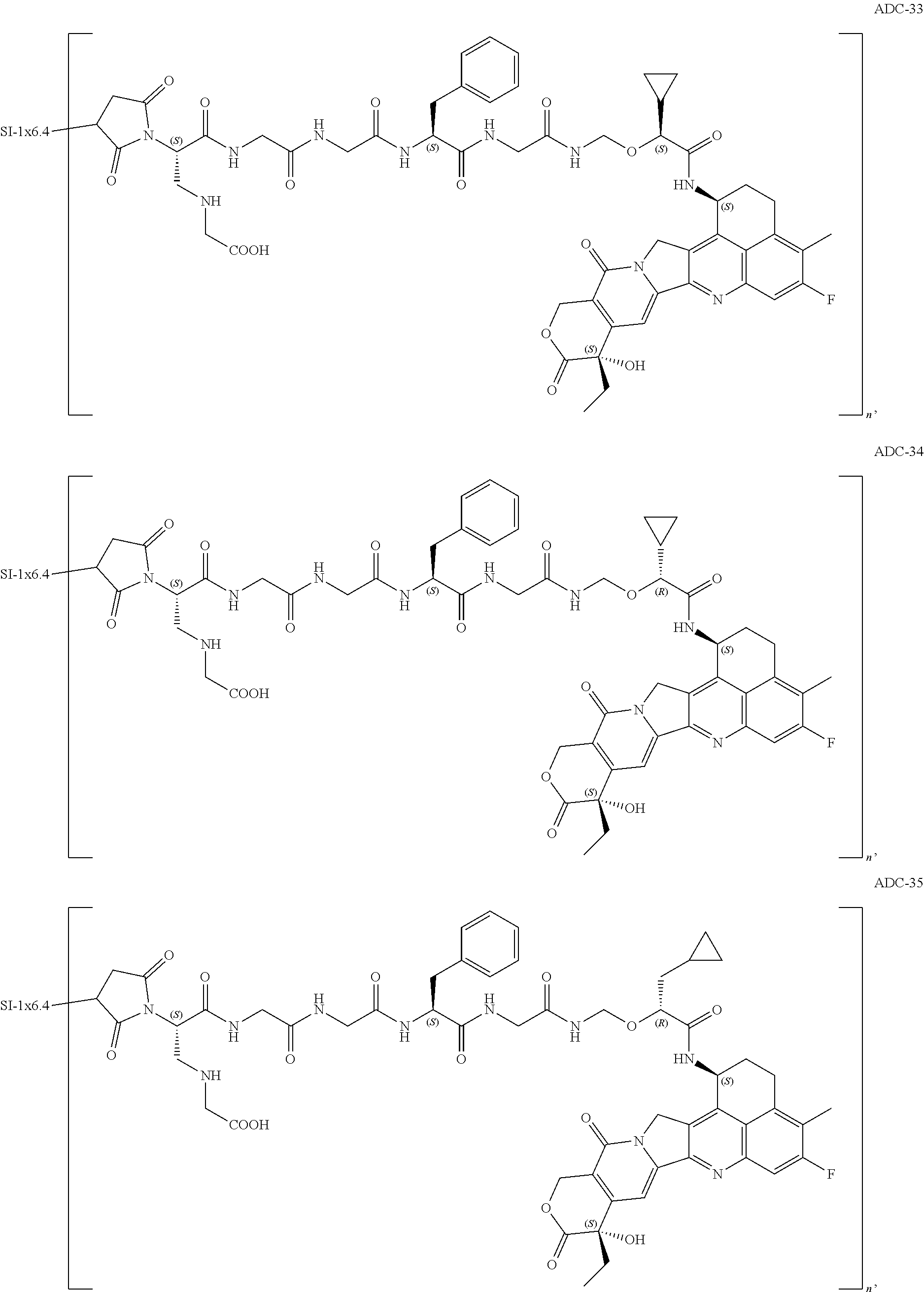

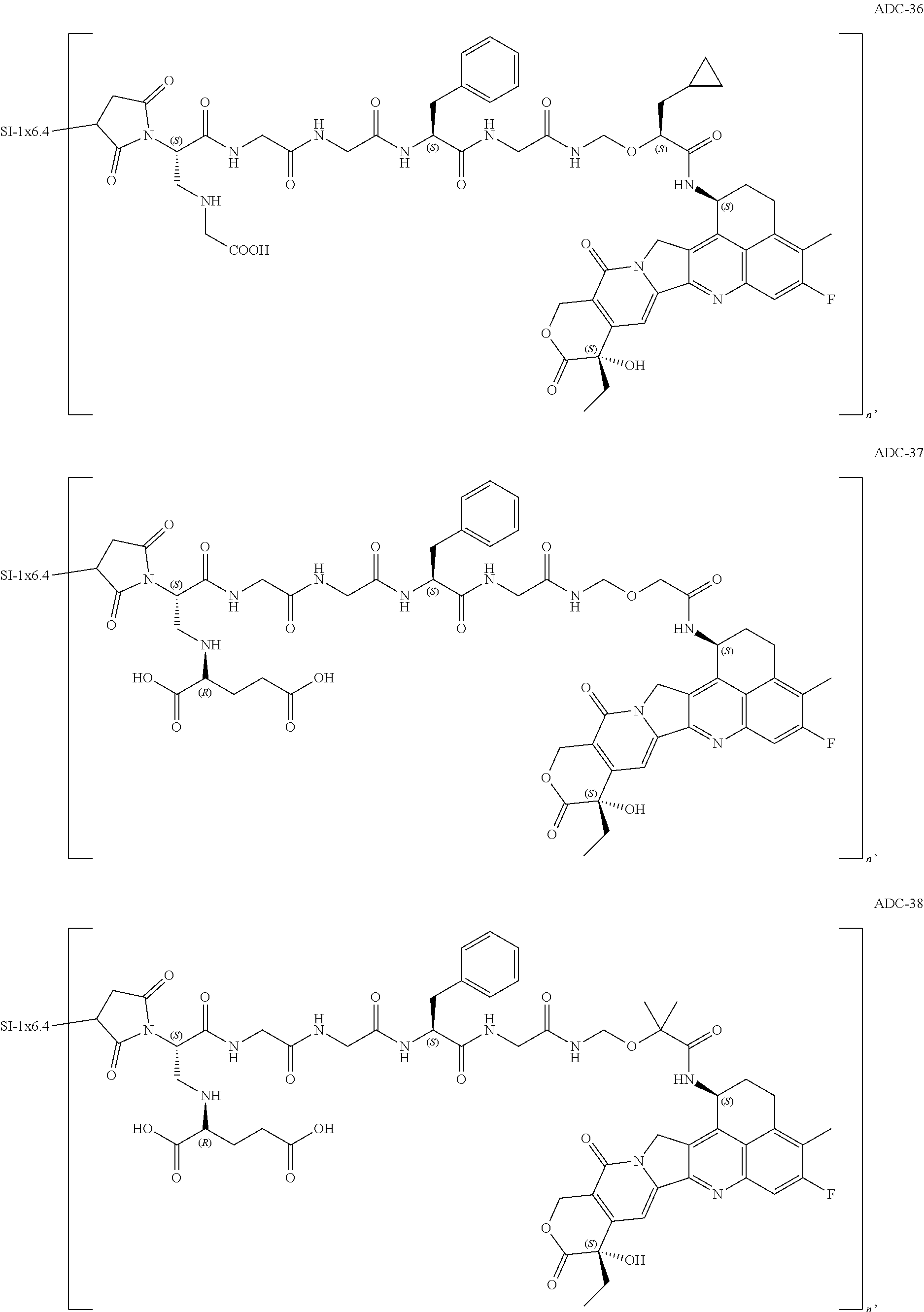

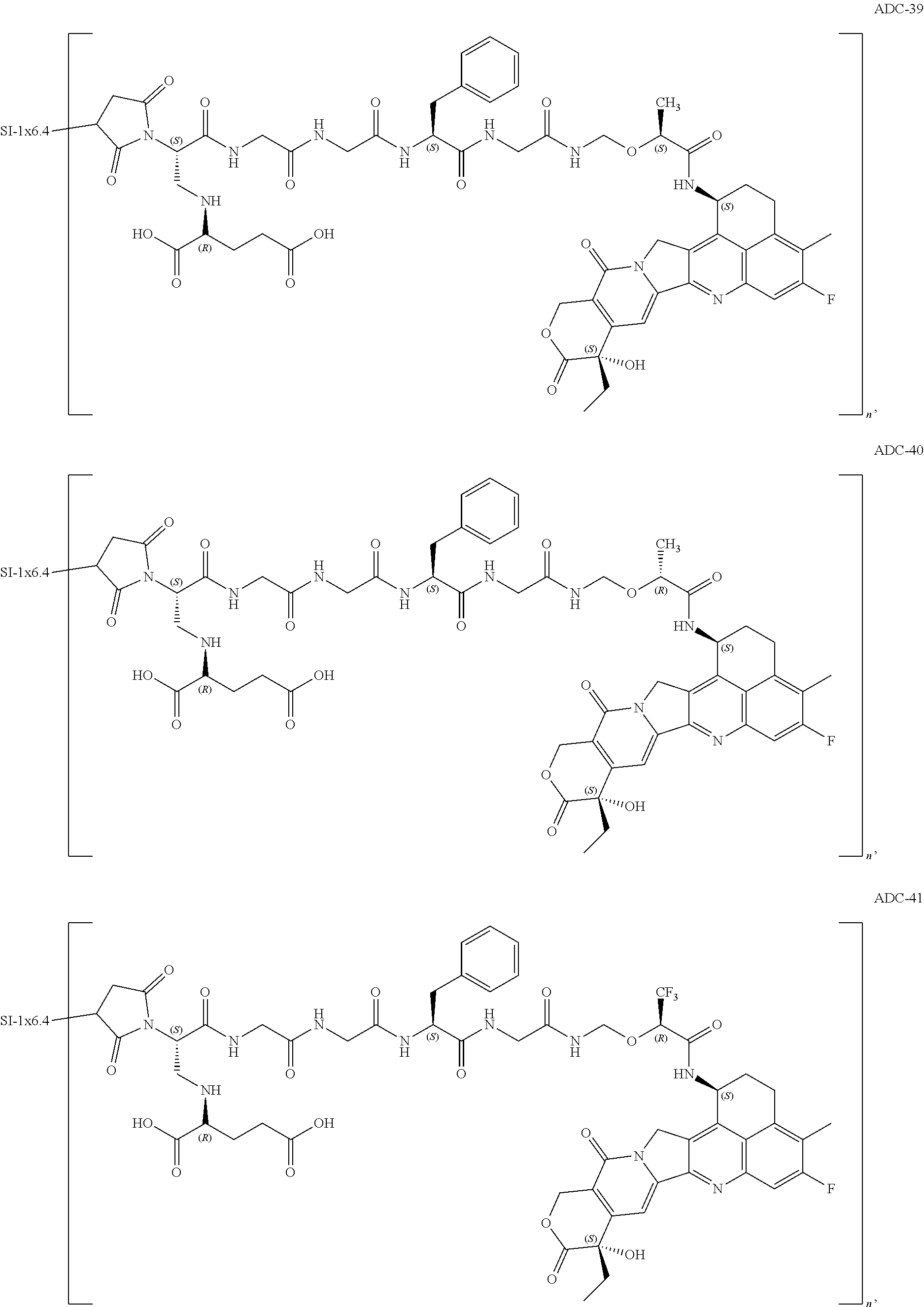

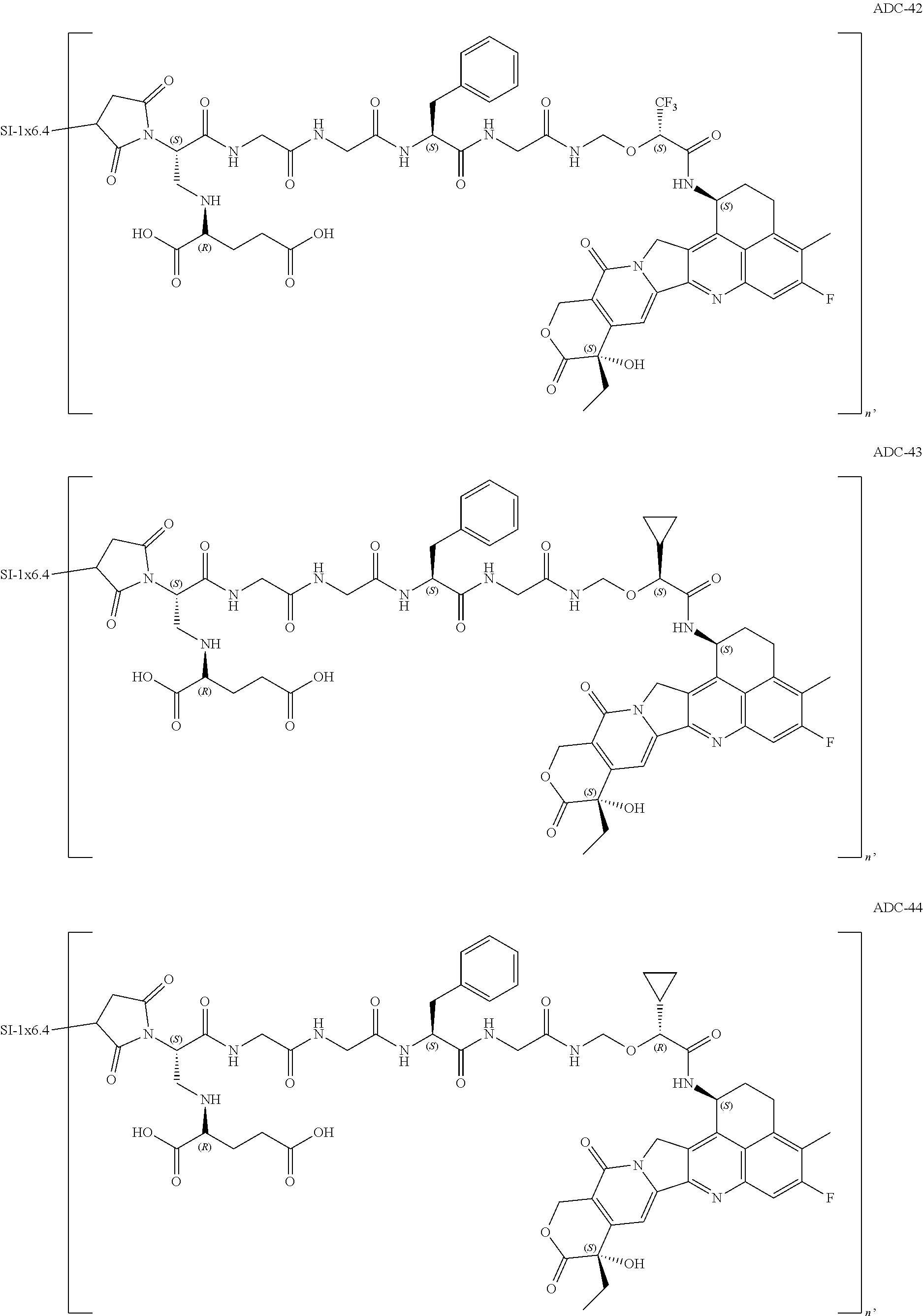

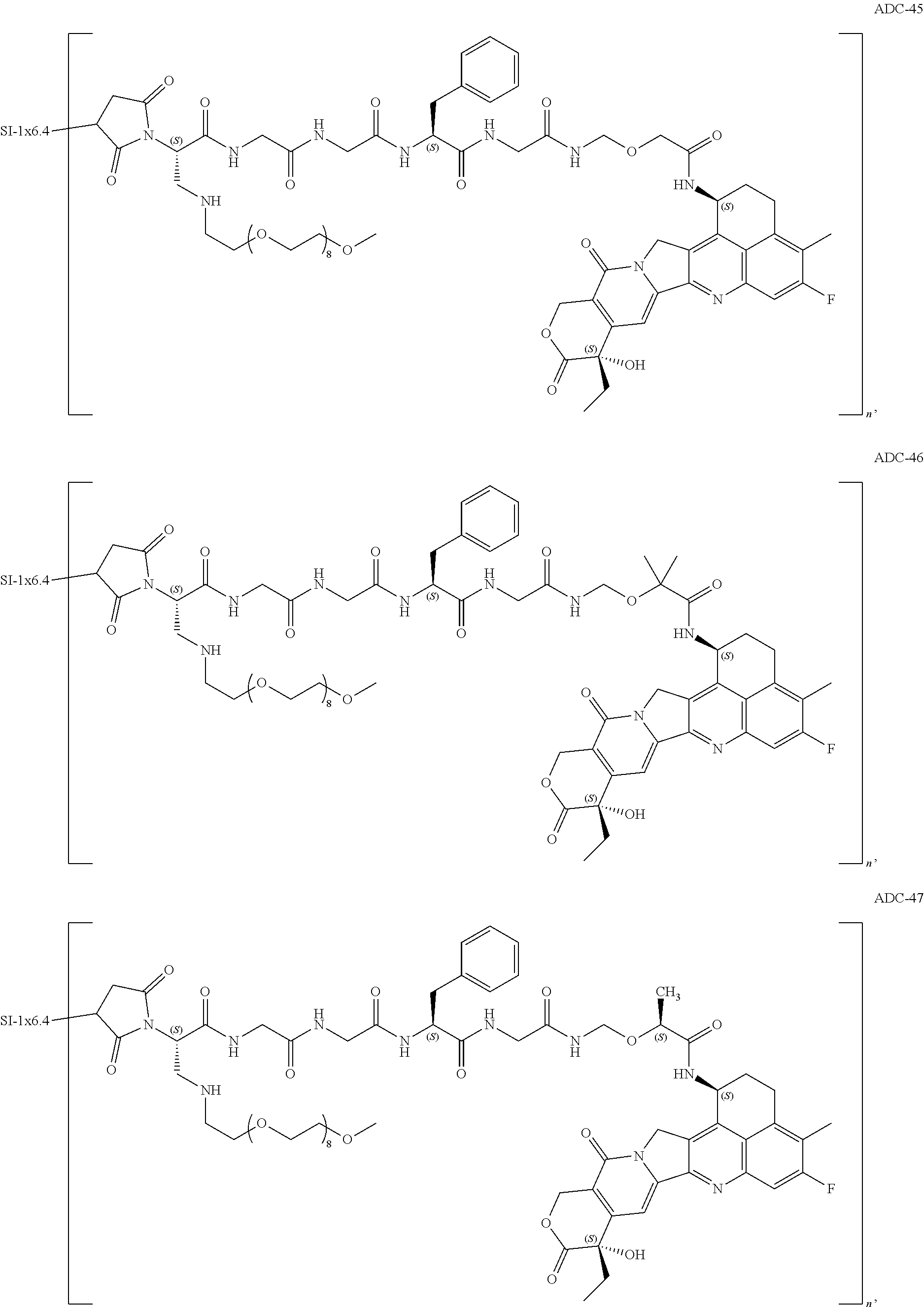

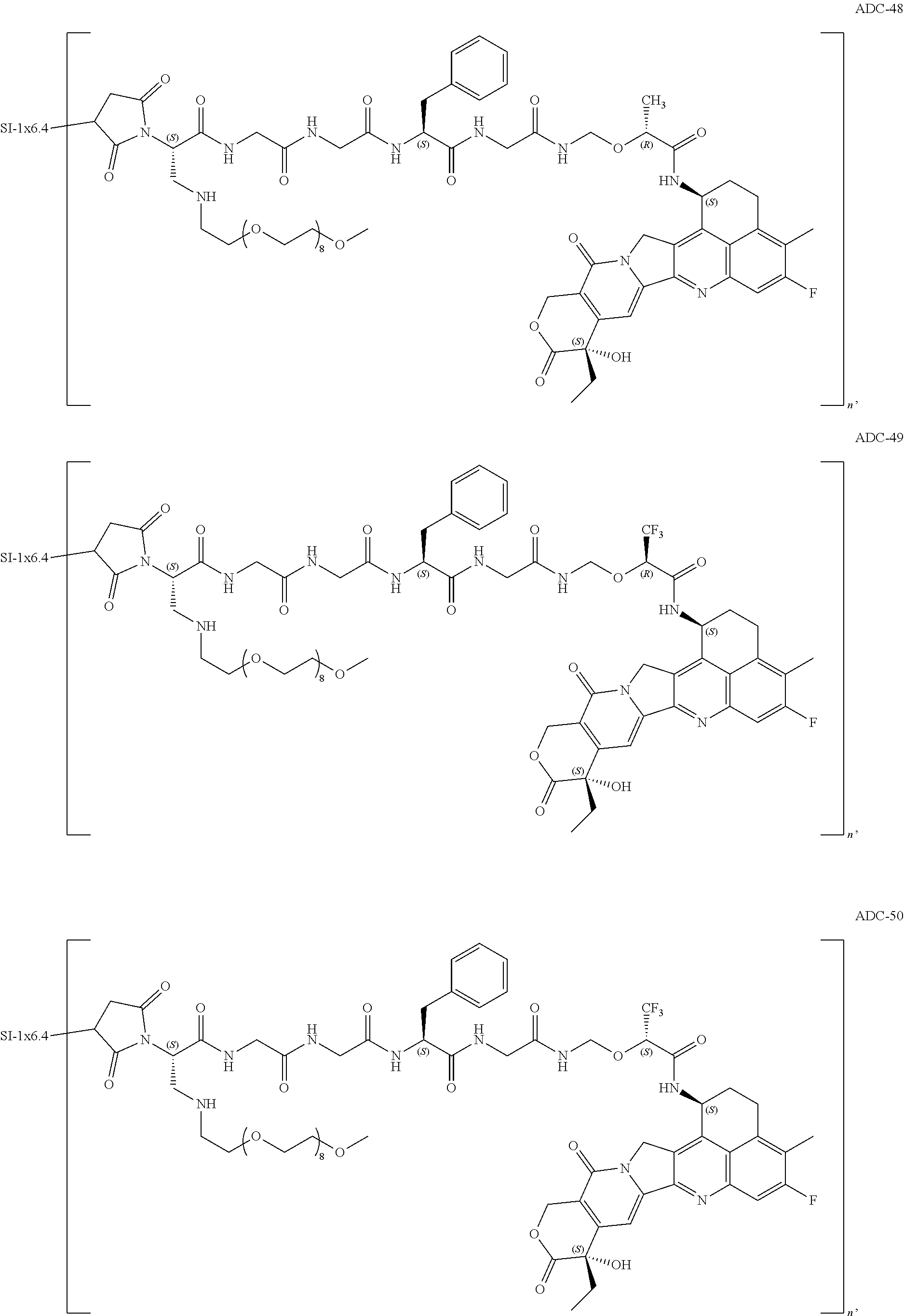

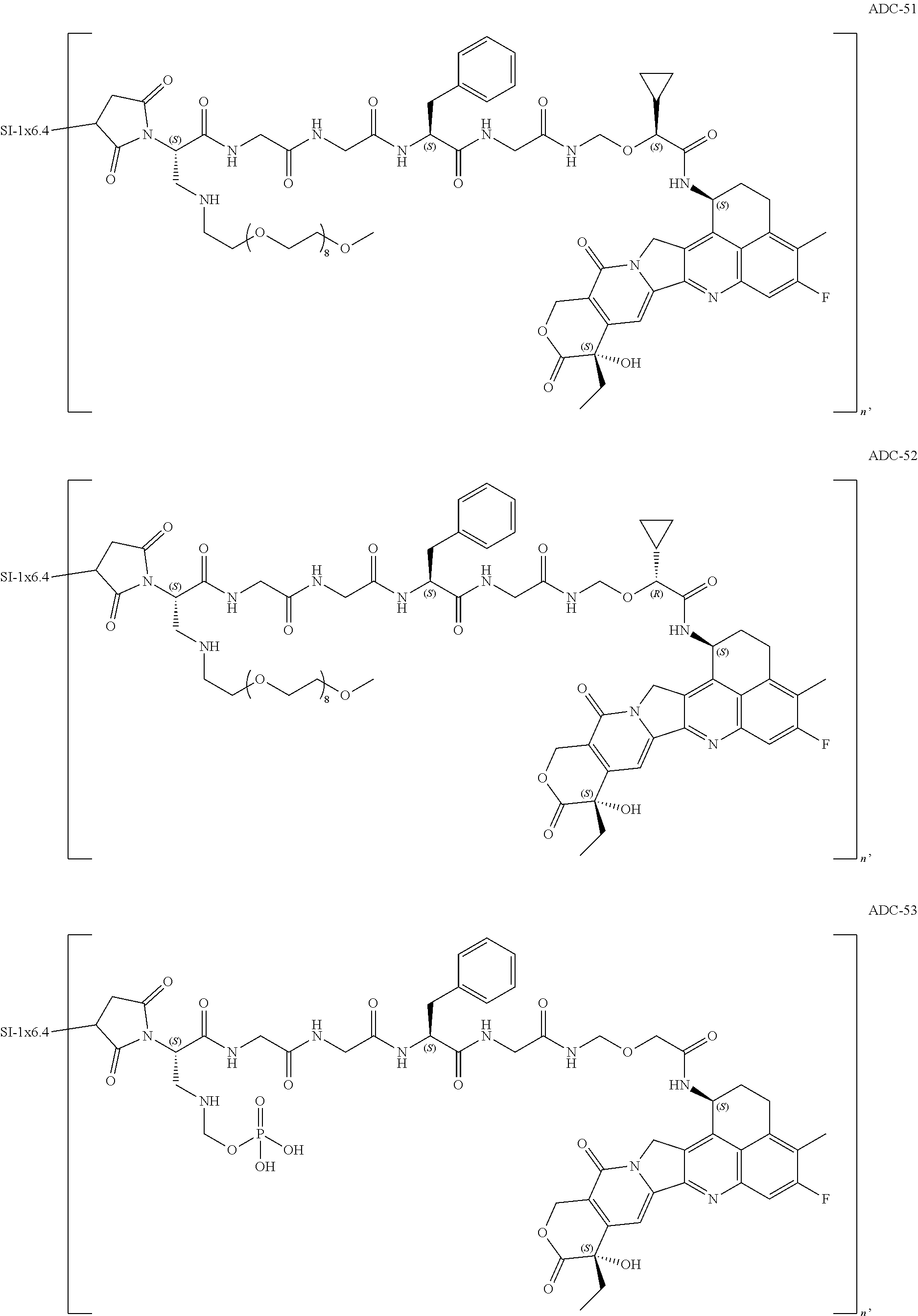

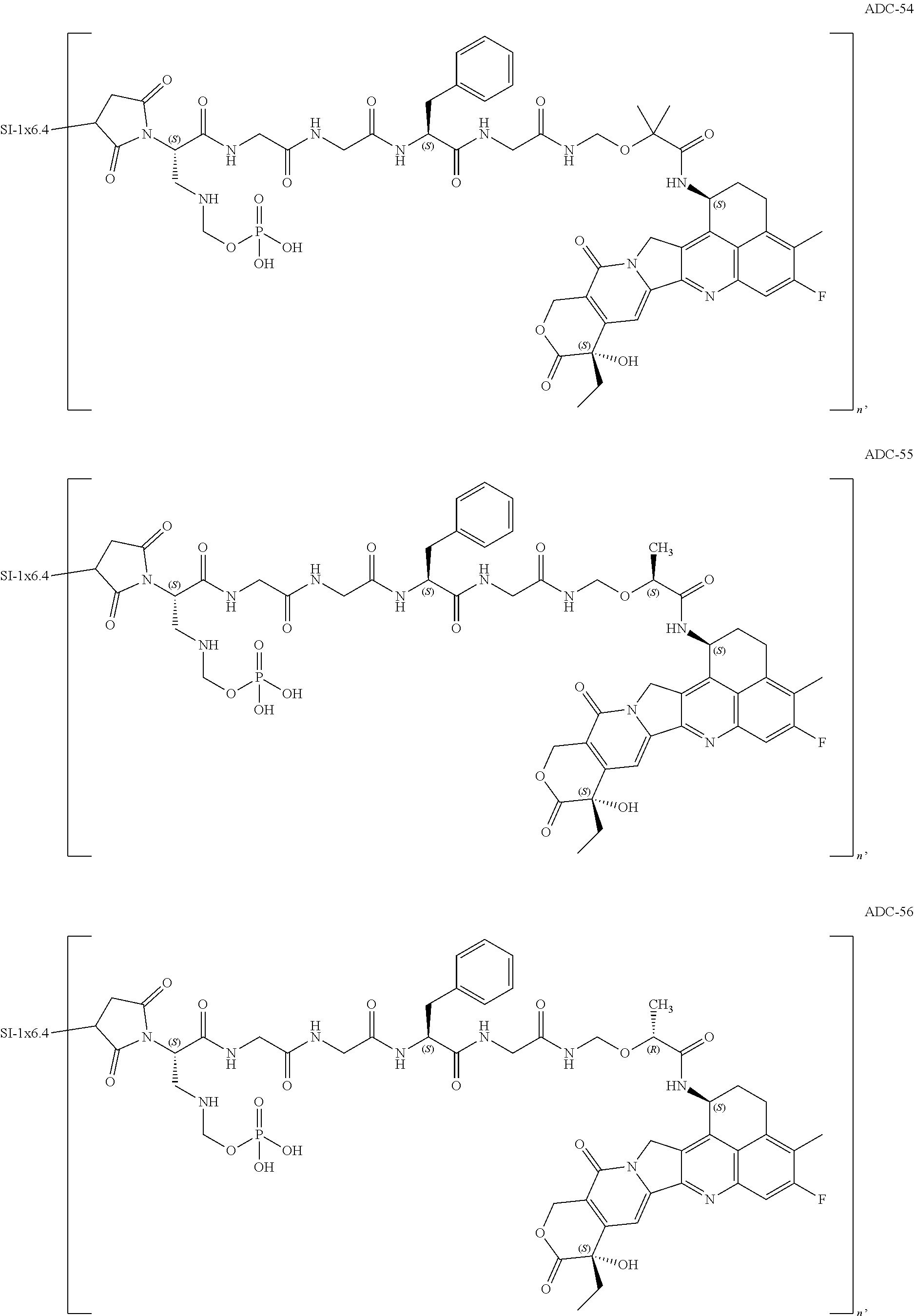

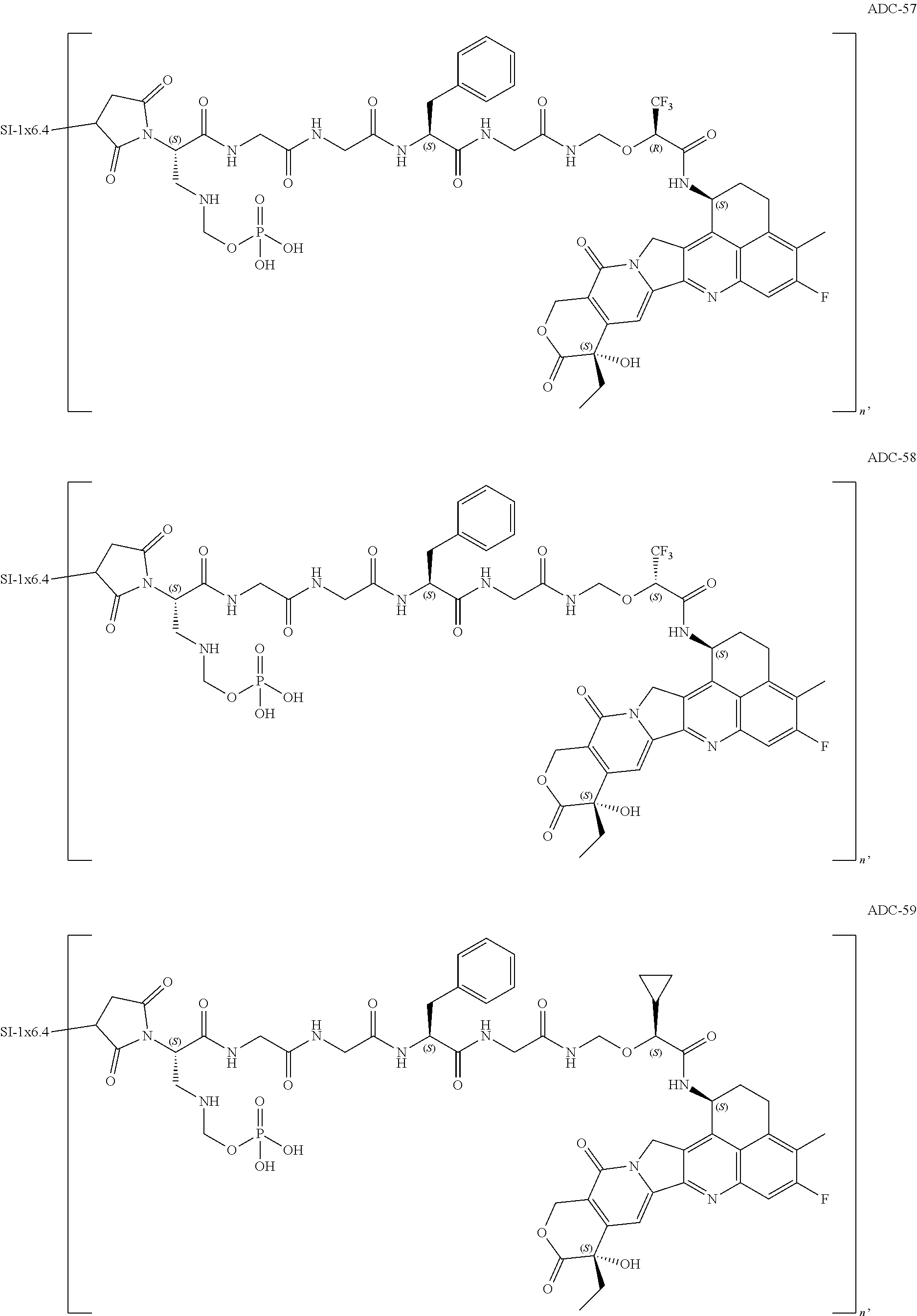

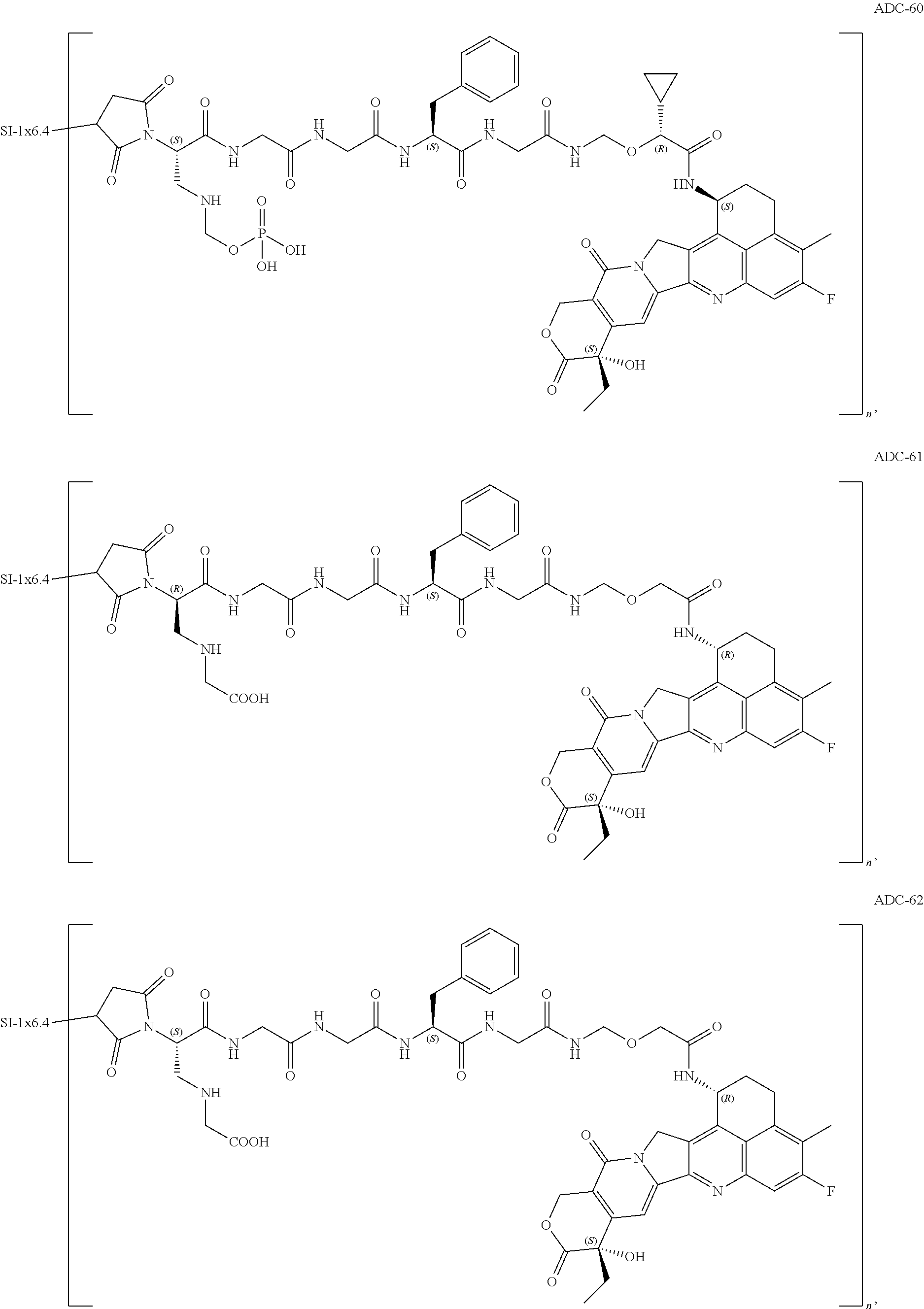

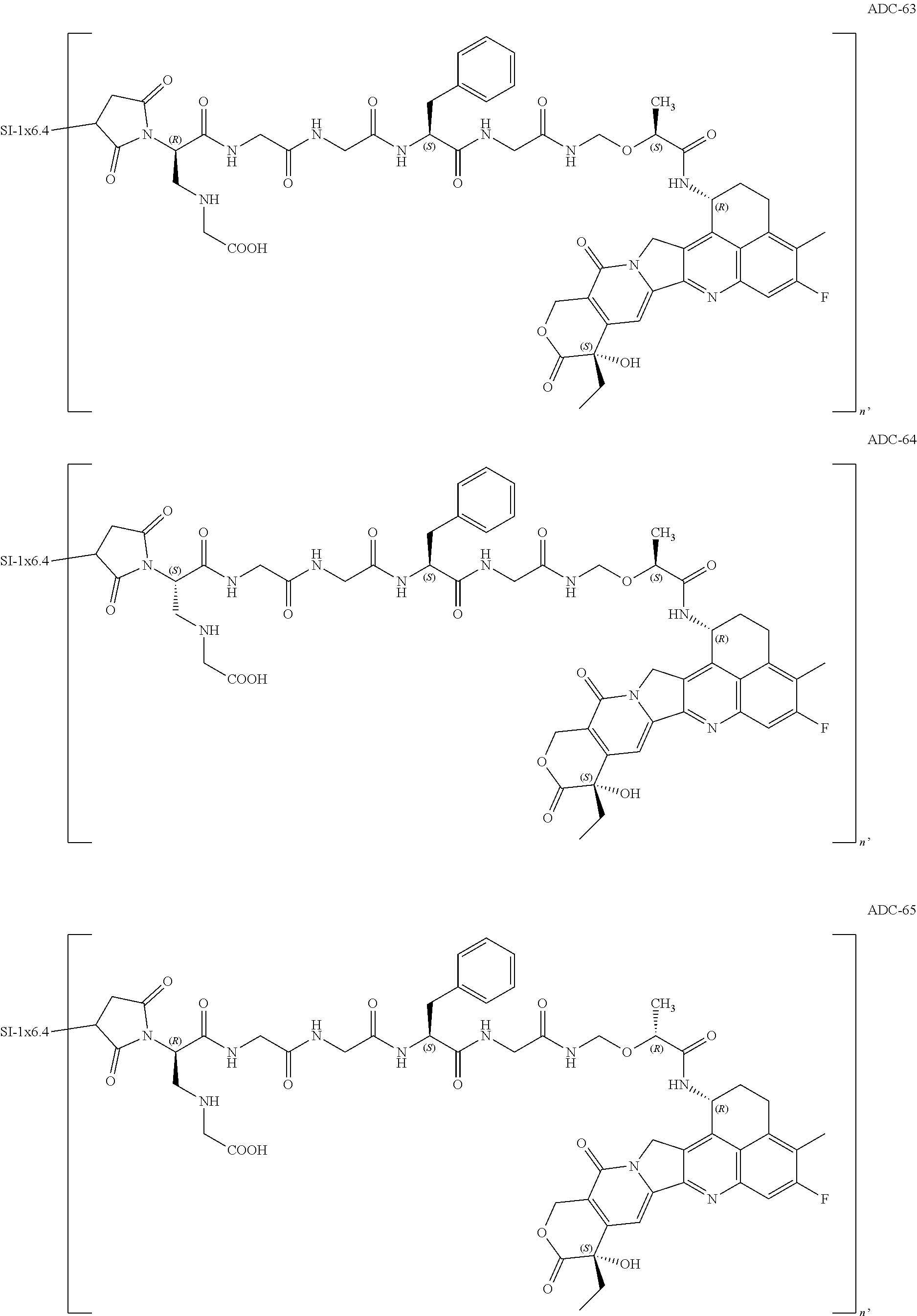

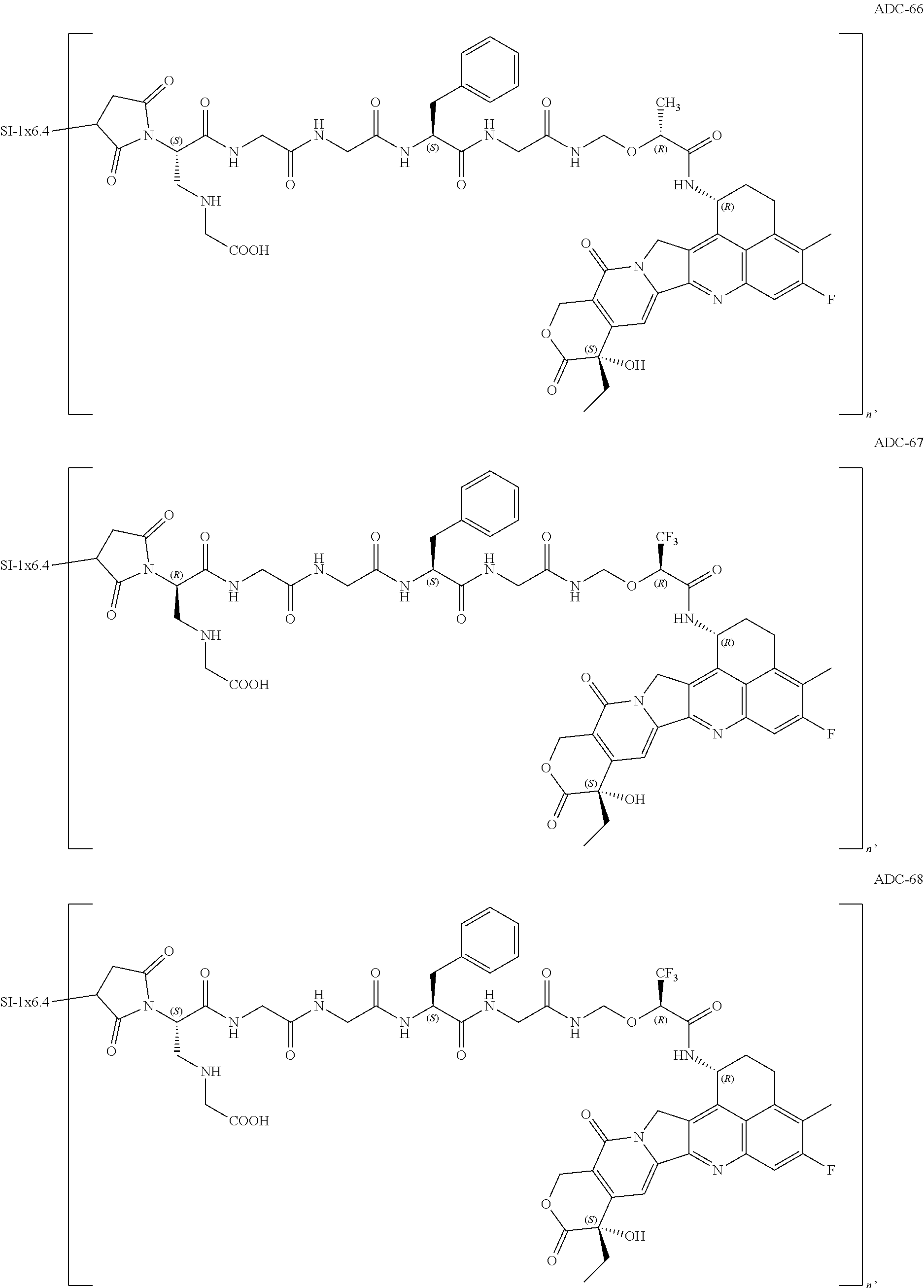

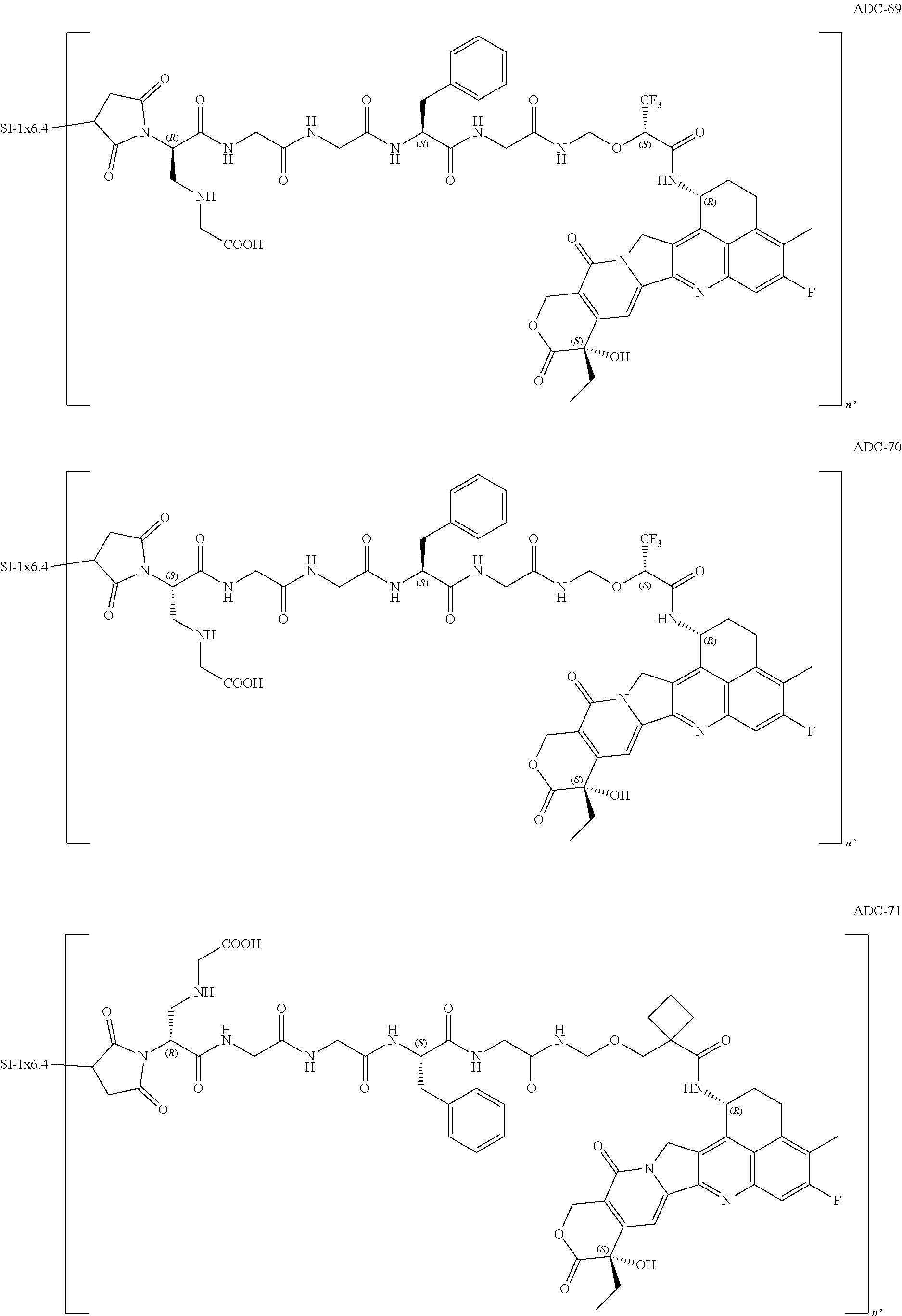

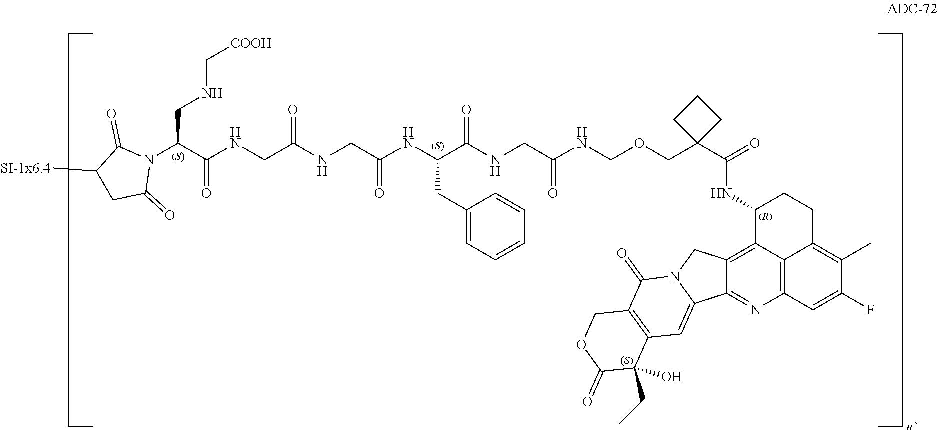

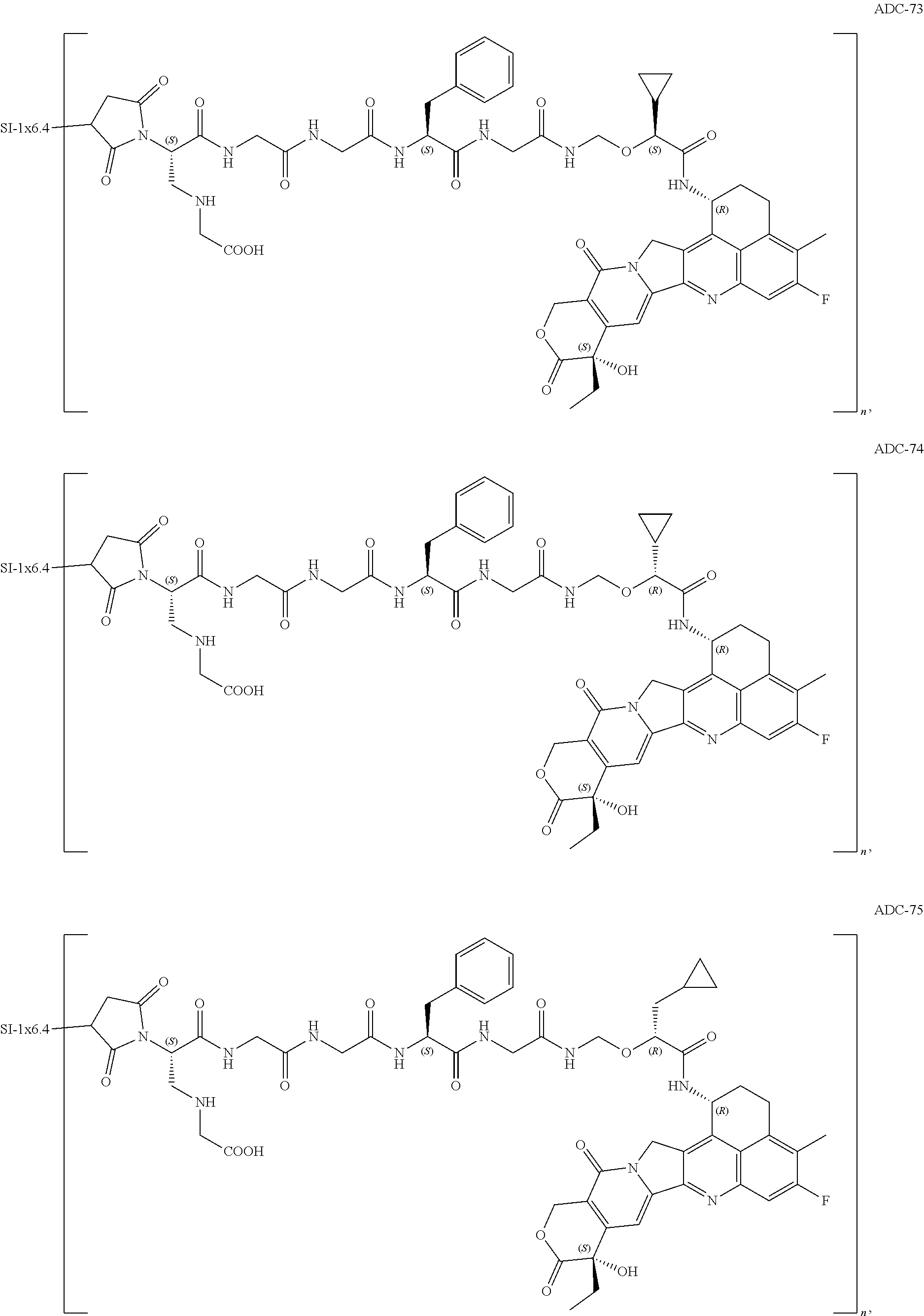

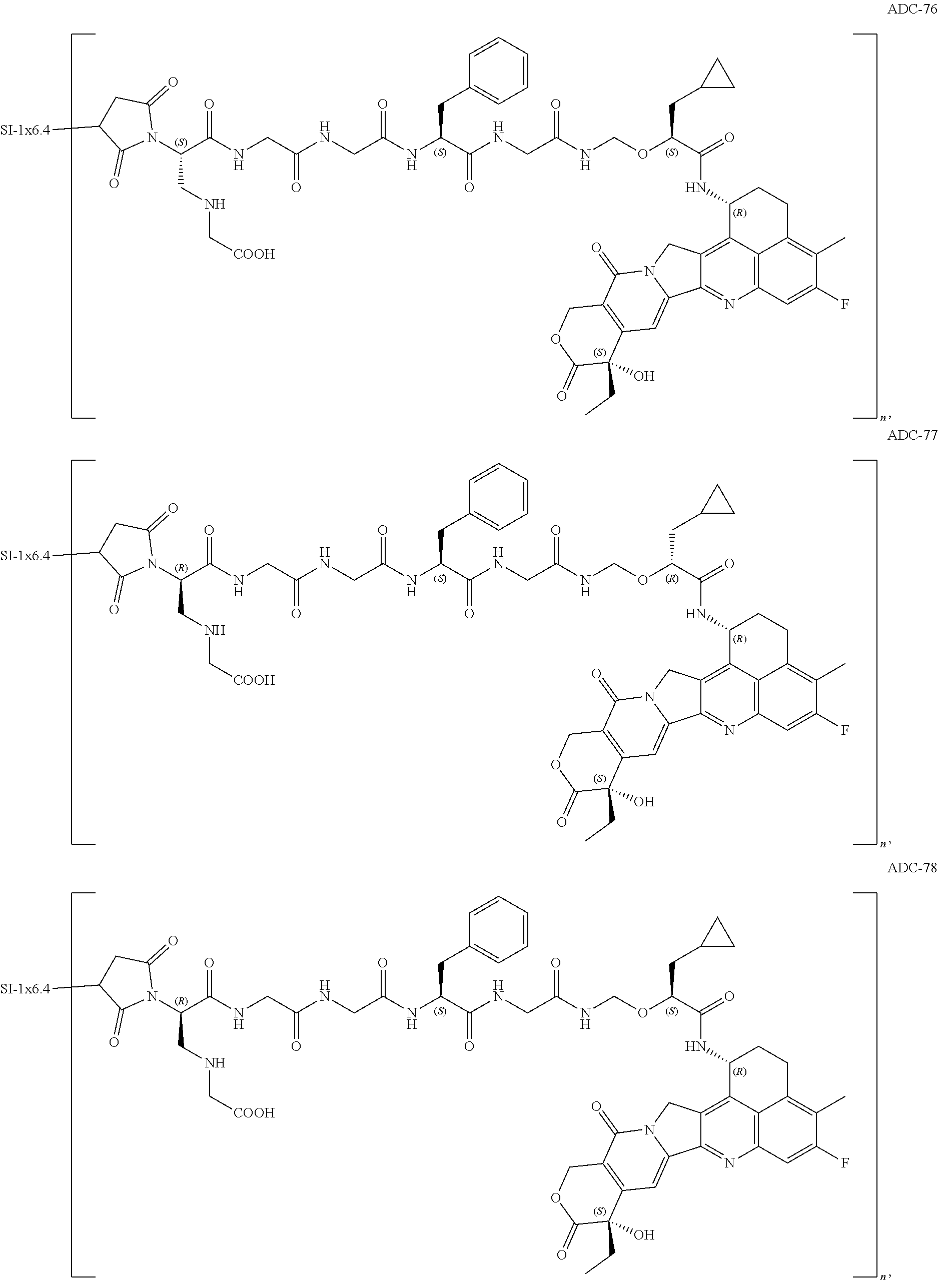

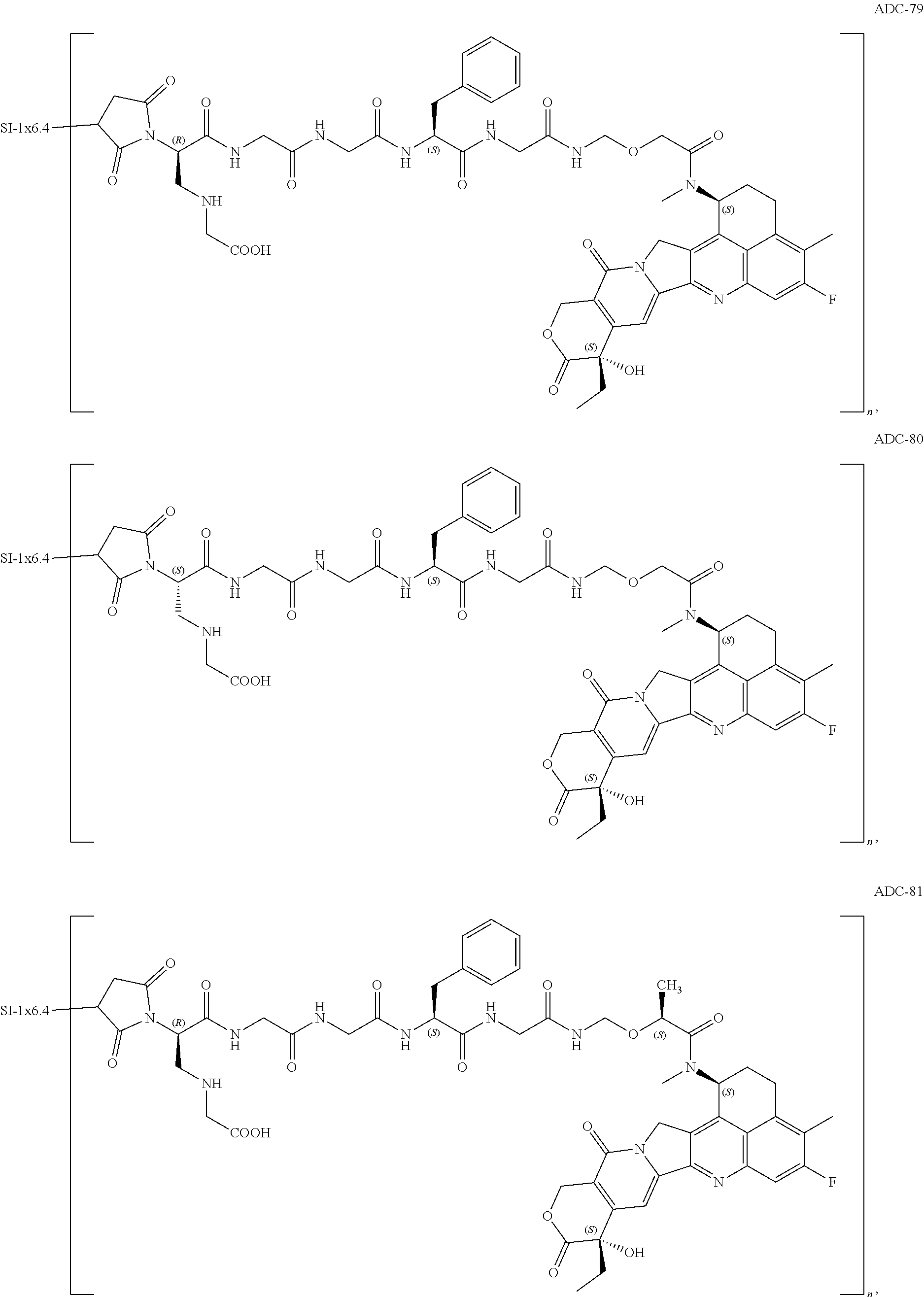

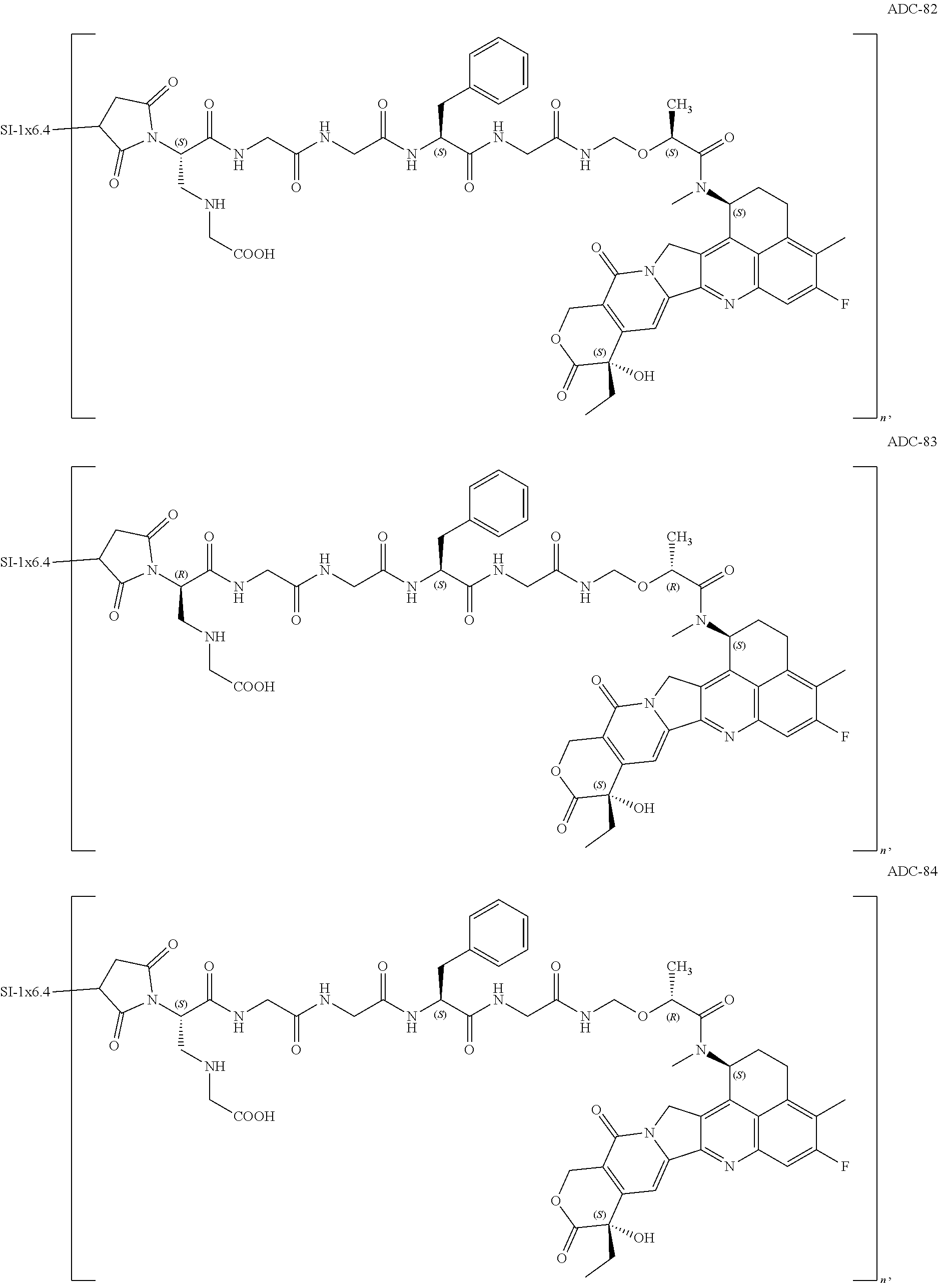

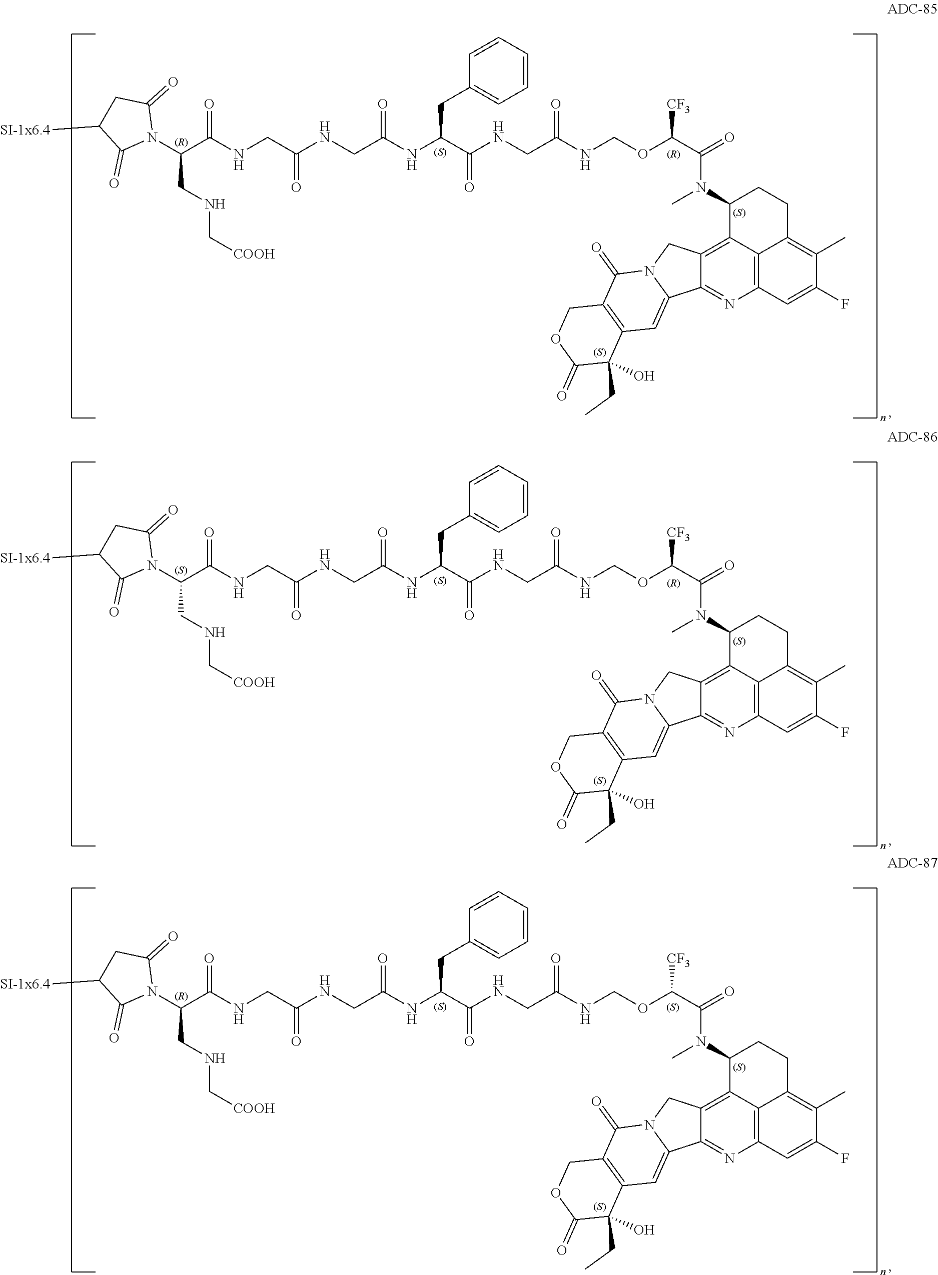

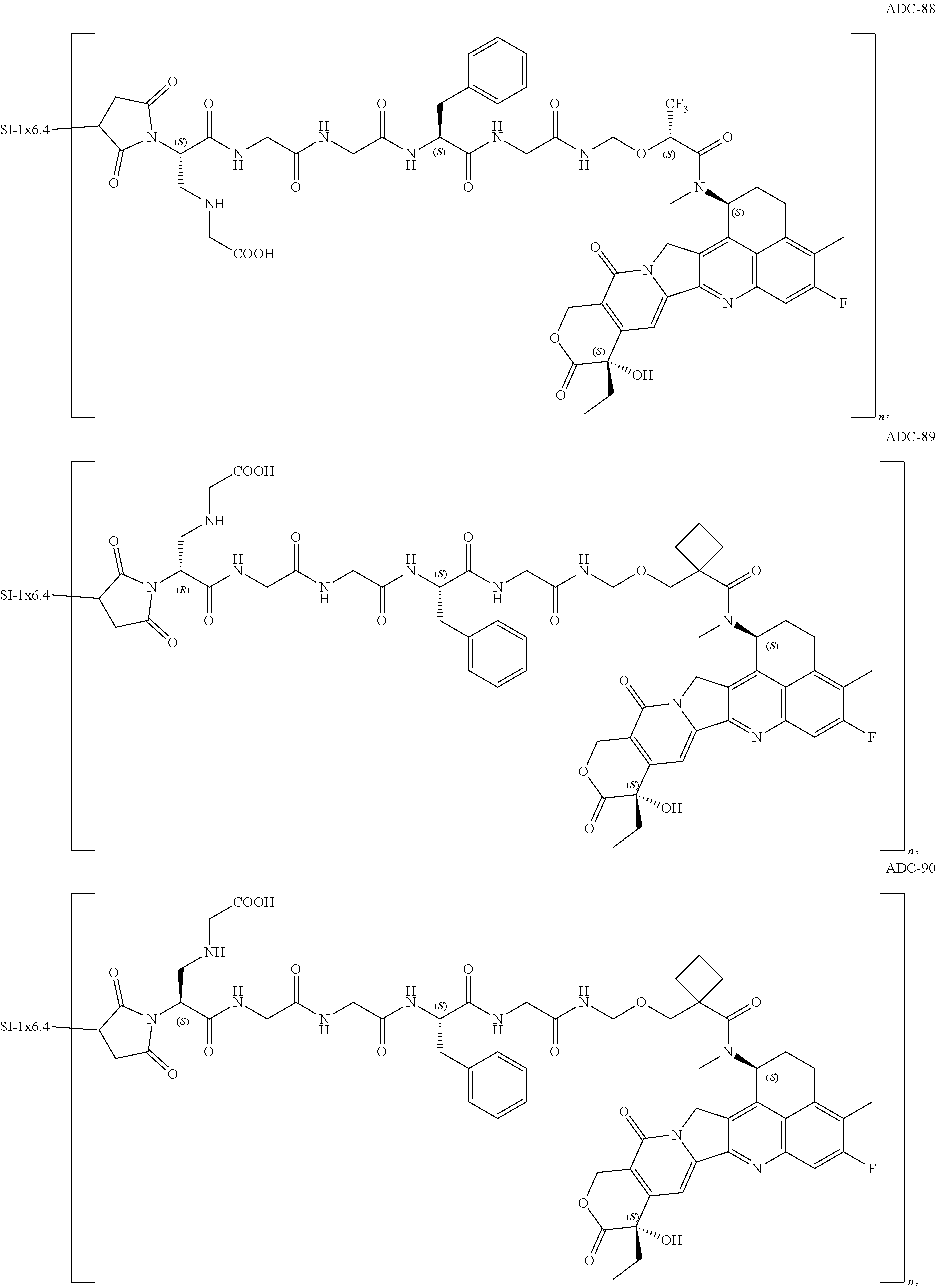

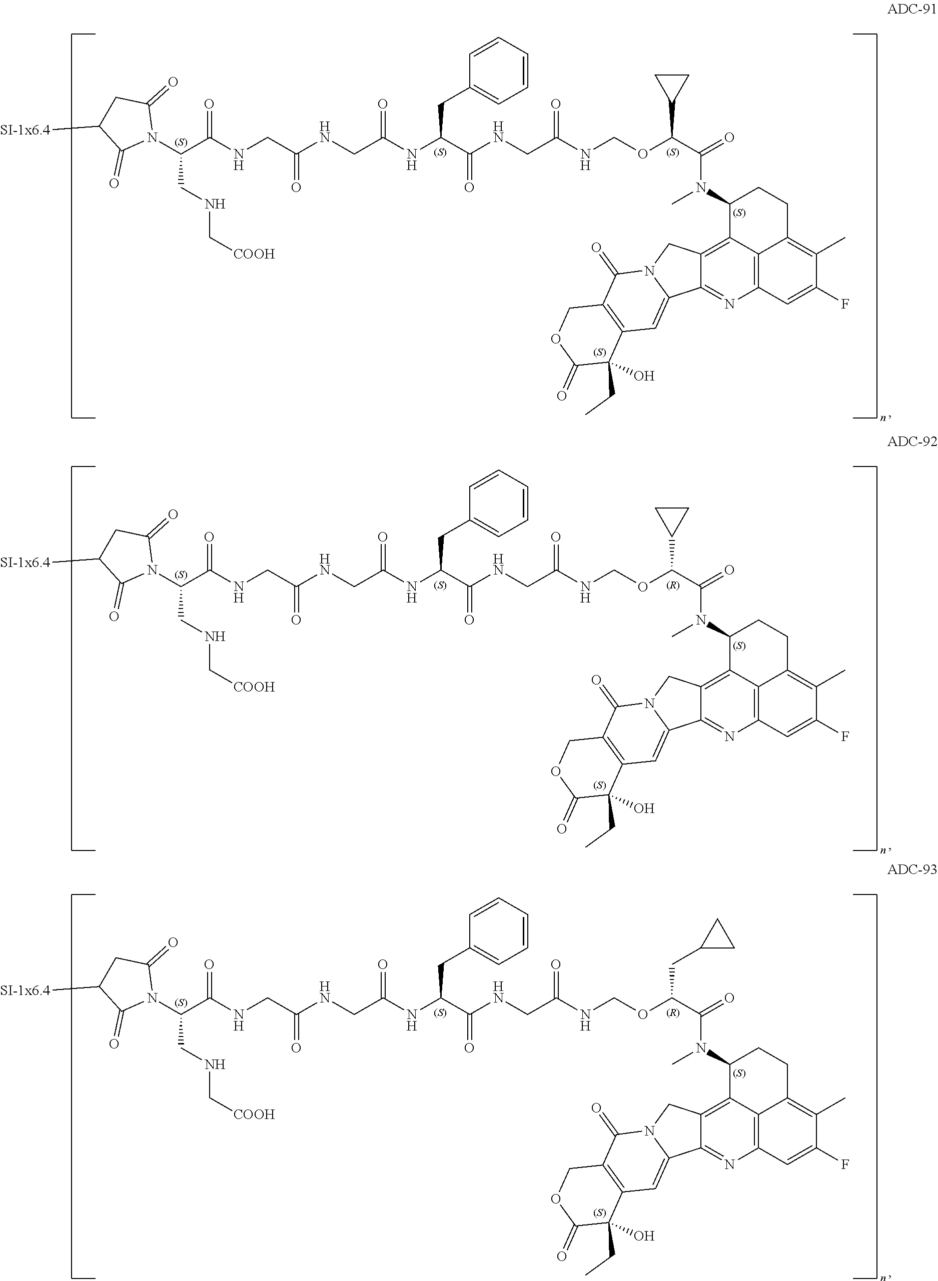

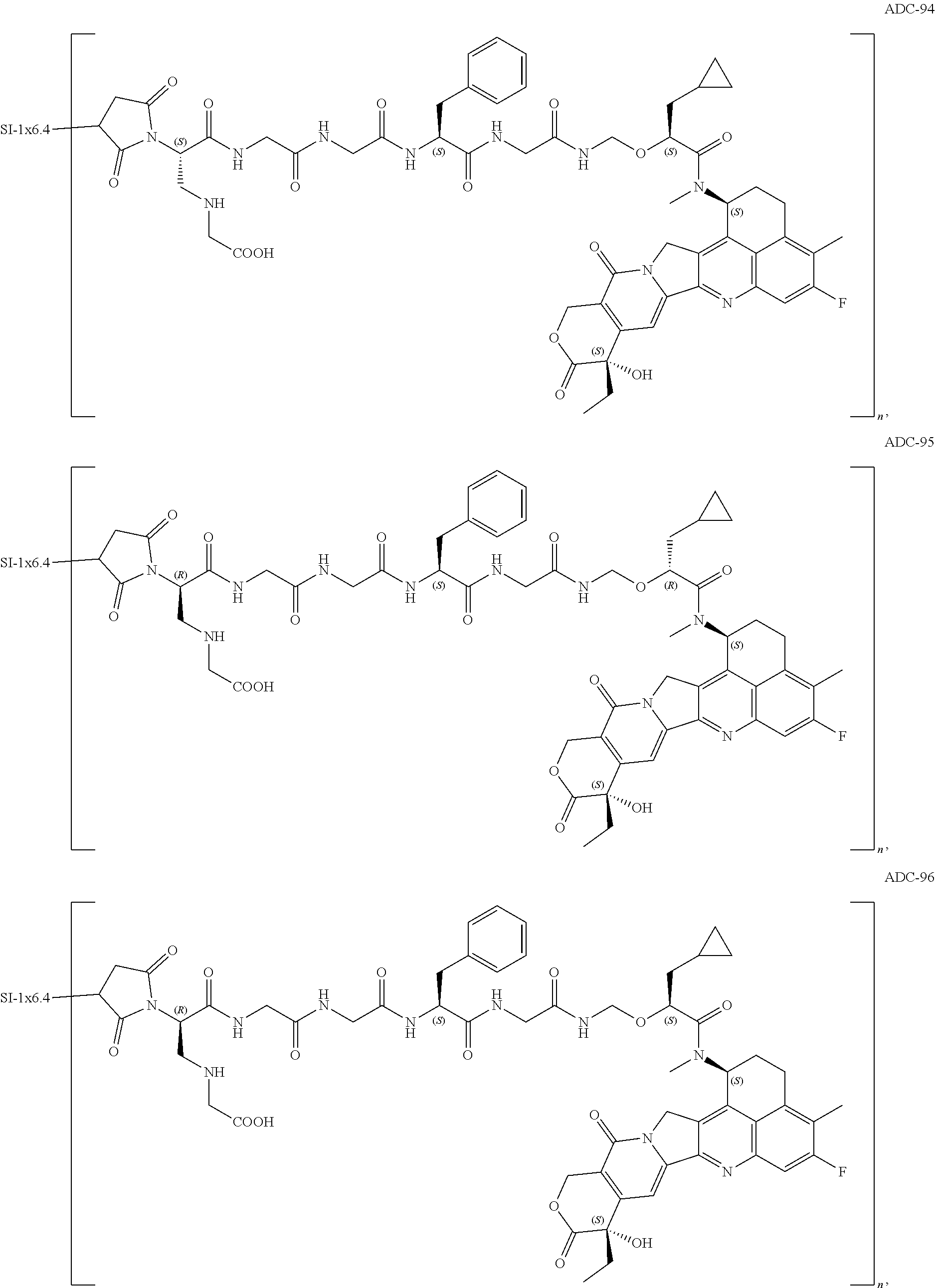

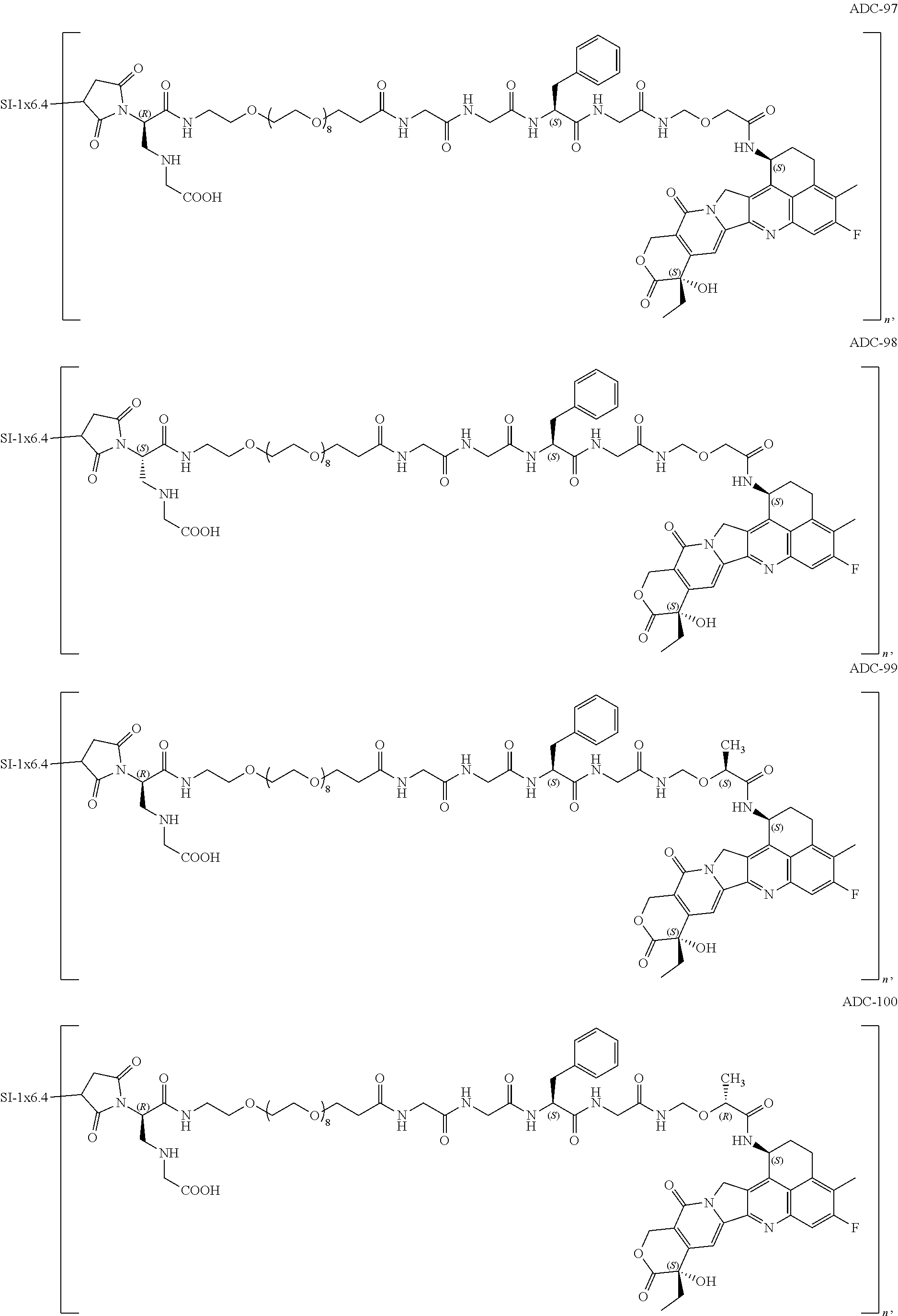

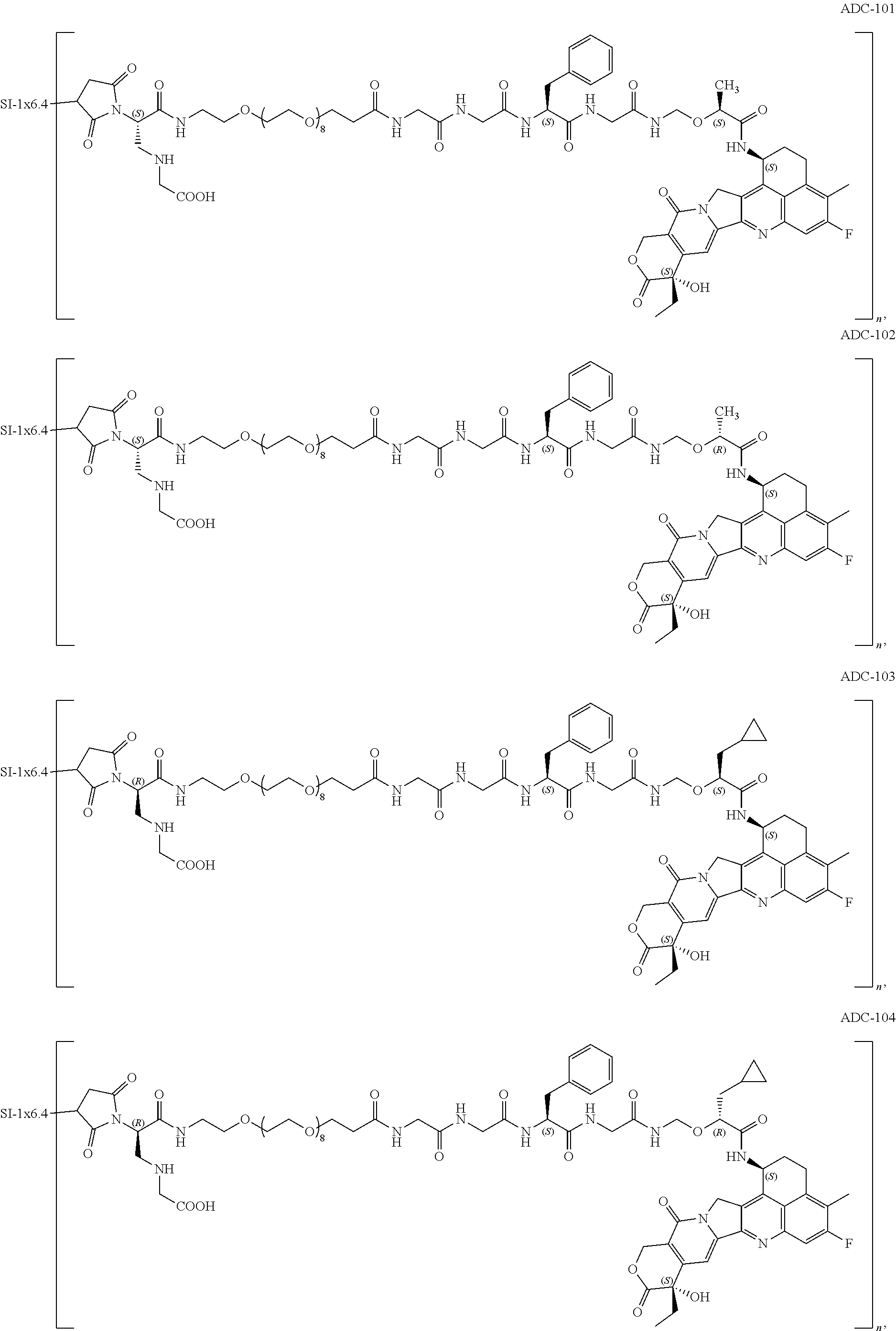

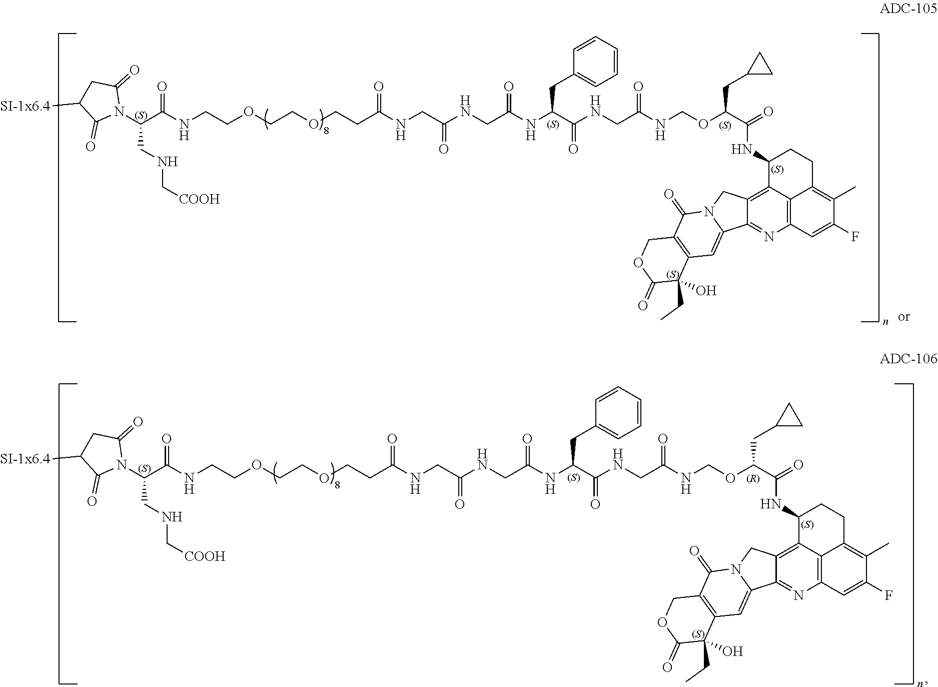

wherein:
SI-1×6.4 is a bispecific antibody or its antigen-binding fragment simultaneously targeting EGFR and HHER3;
n is selected from the integers 1-10 (i.e., 1, 2, 3, 4, 5, 6, 7, 8, 9, or 10).

In some embodiments of the first aspect, the second aspect and the fourth aspect of the present invention, the disclosure of said ligand-camptothecin derivative conjugate, or a pharmaceutically acceptable salt or solvate thereof, is characterized in that said ligand-camptothecin derivative conjugate, or a pharmaceutically acceptable salt or solvate thereof, is selected without limitation from the following structures or succinimide open-ring structures thereof or isomers thereof,

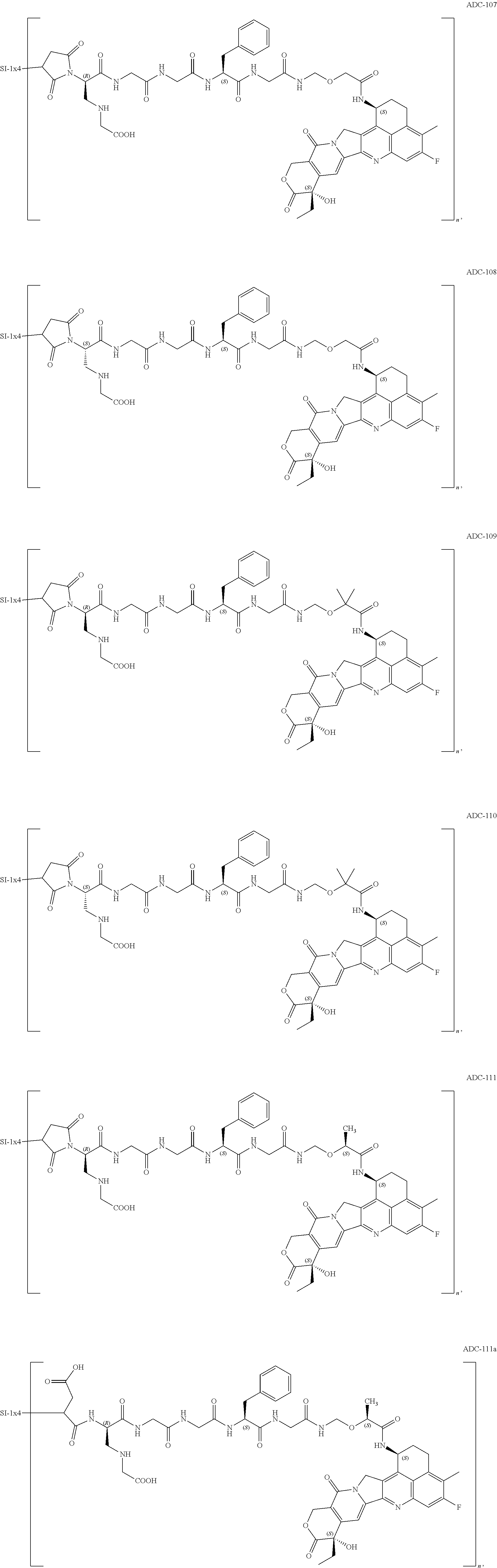

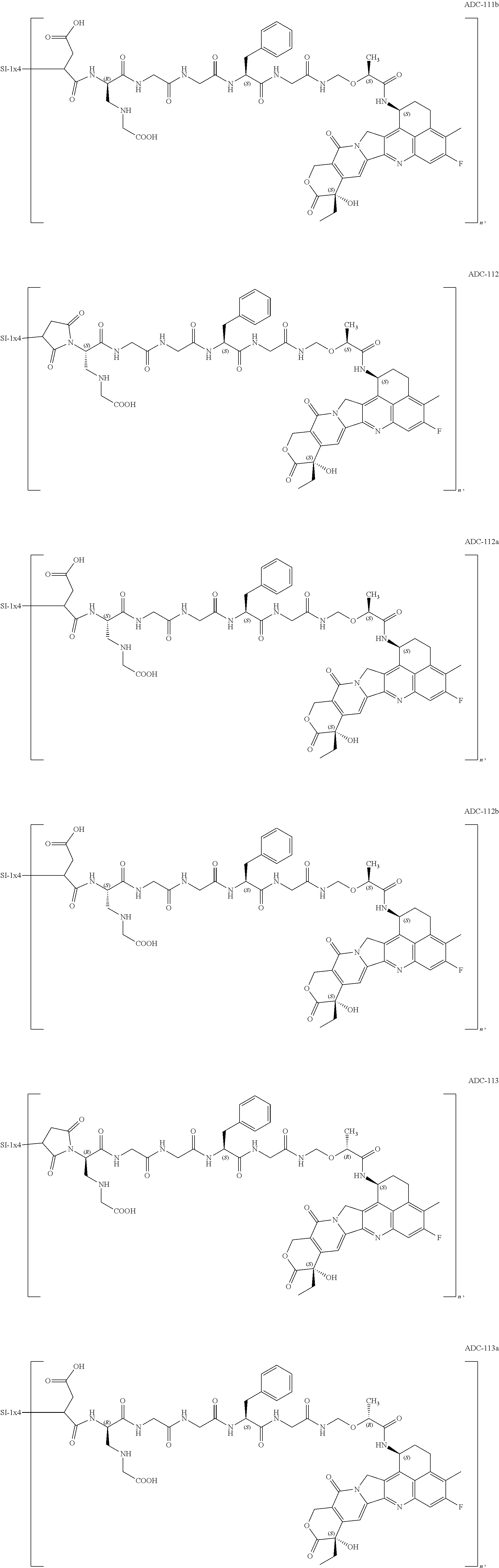

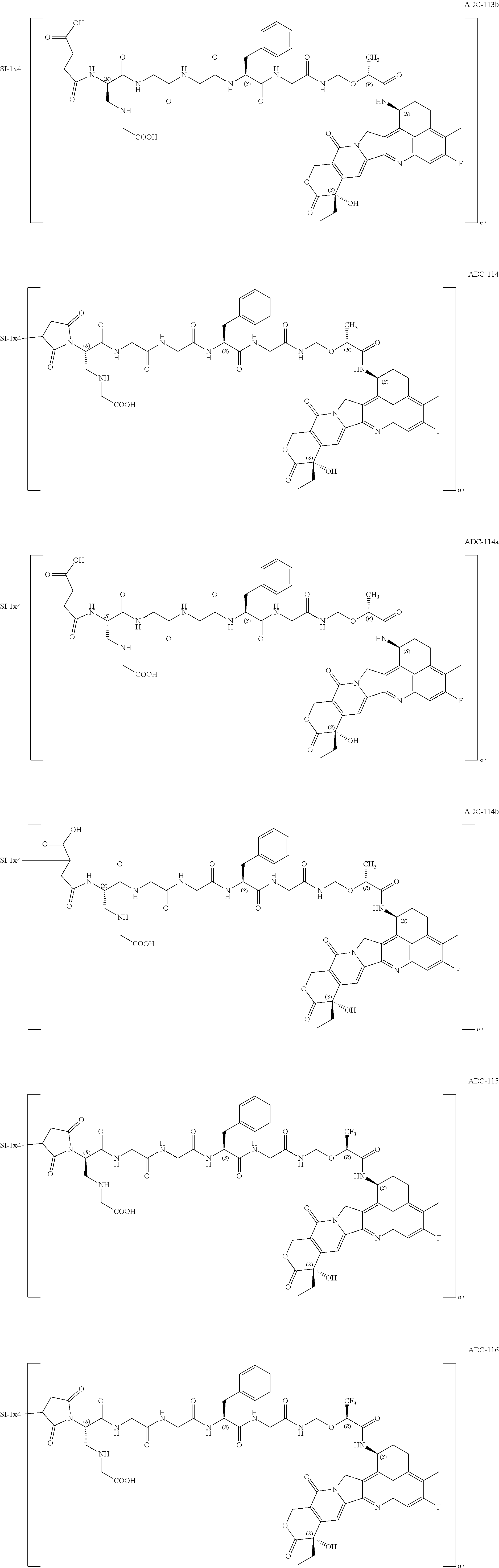

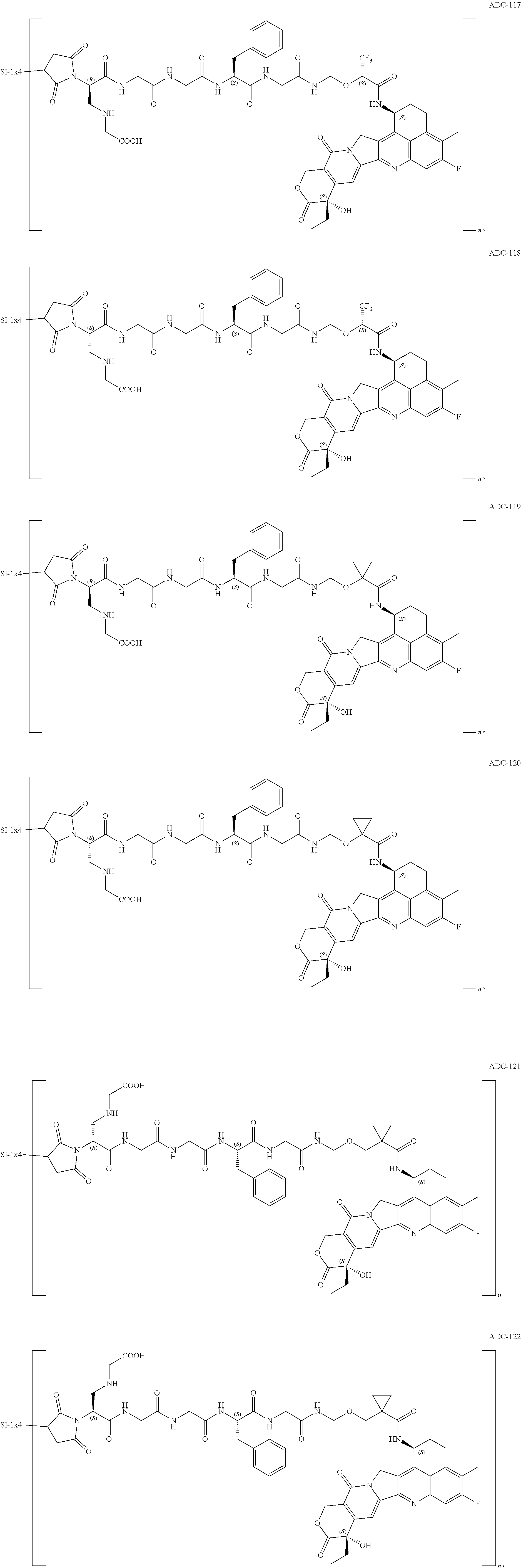

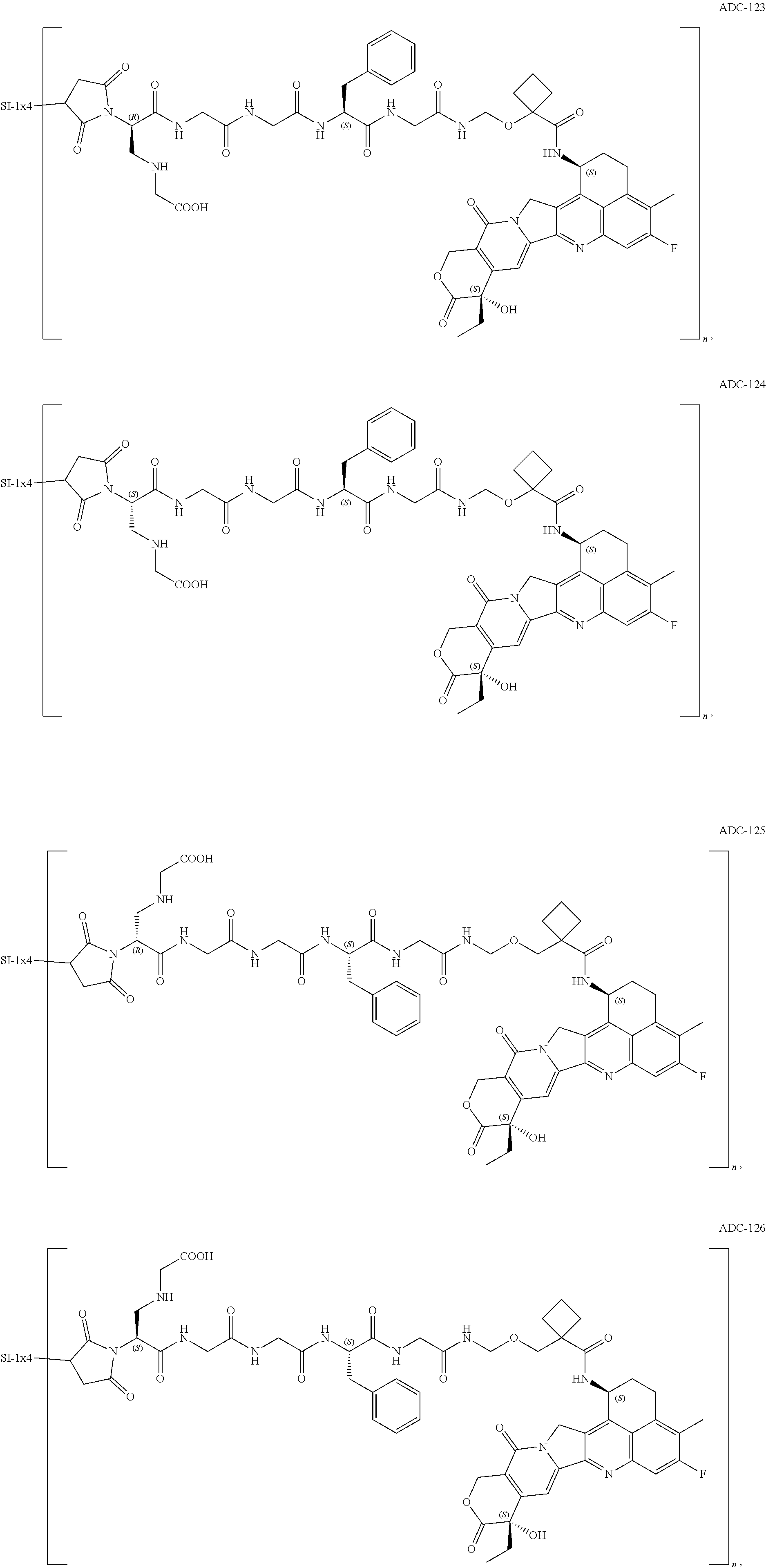

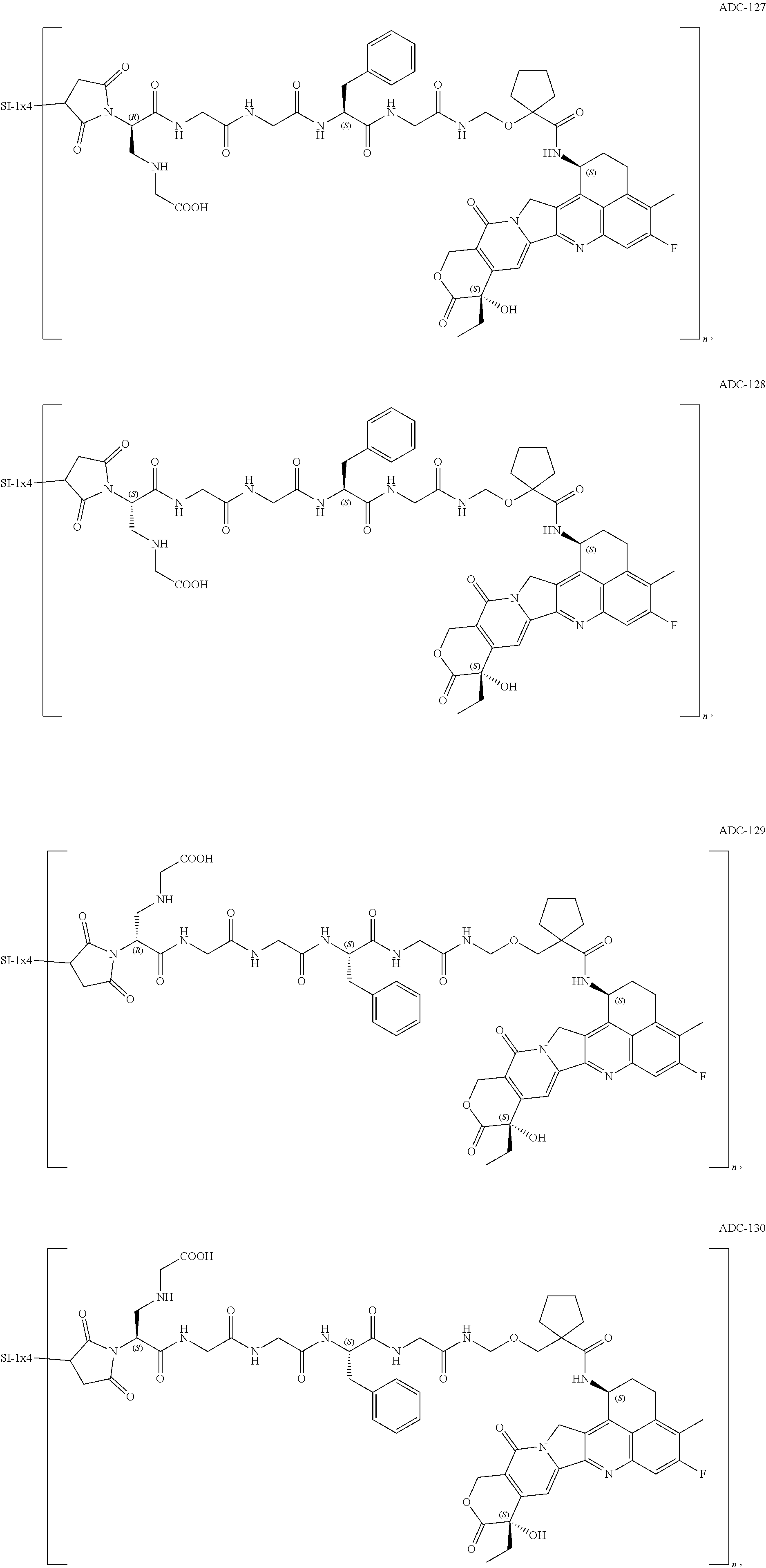

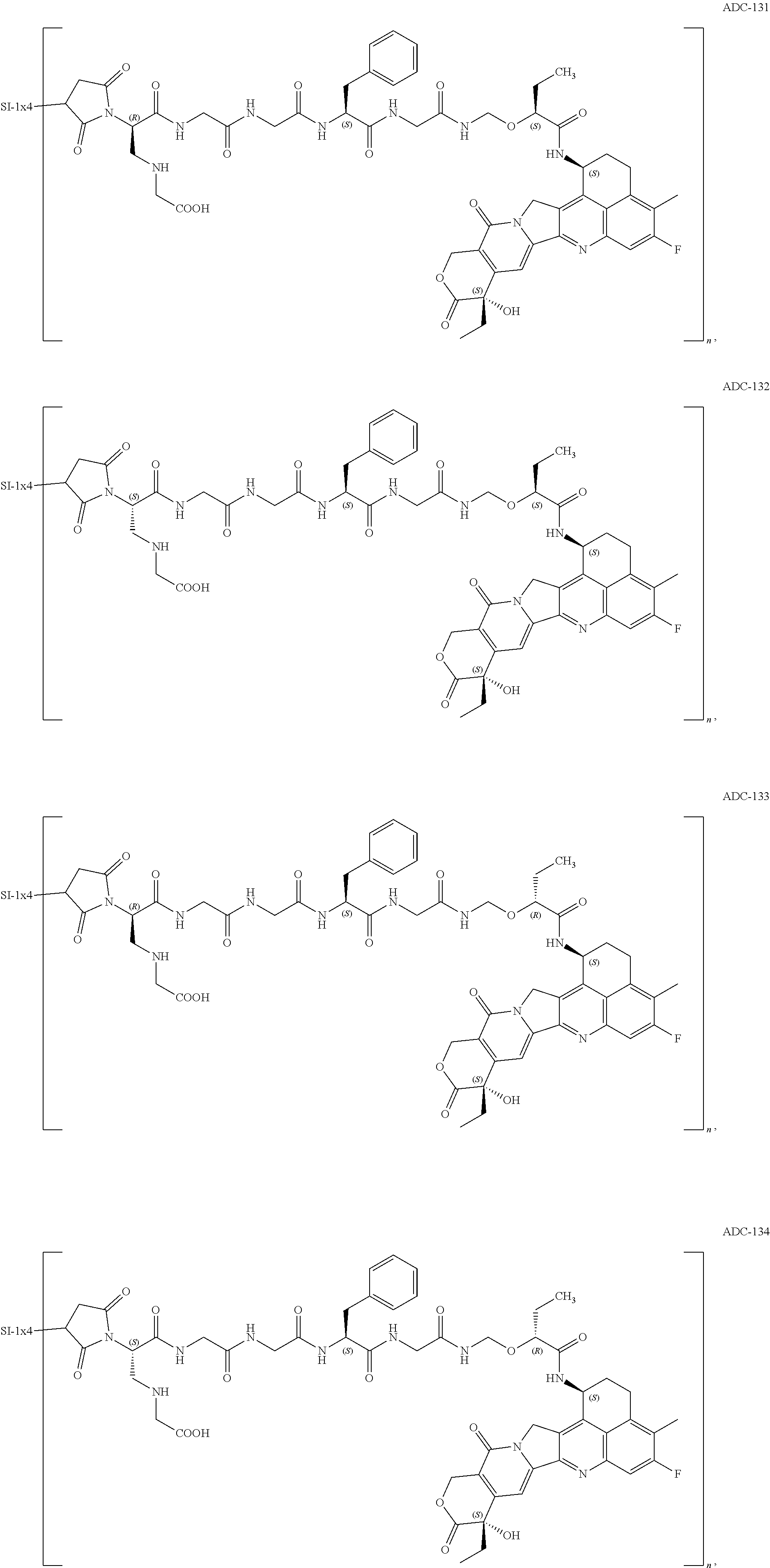

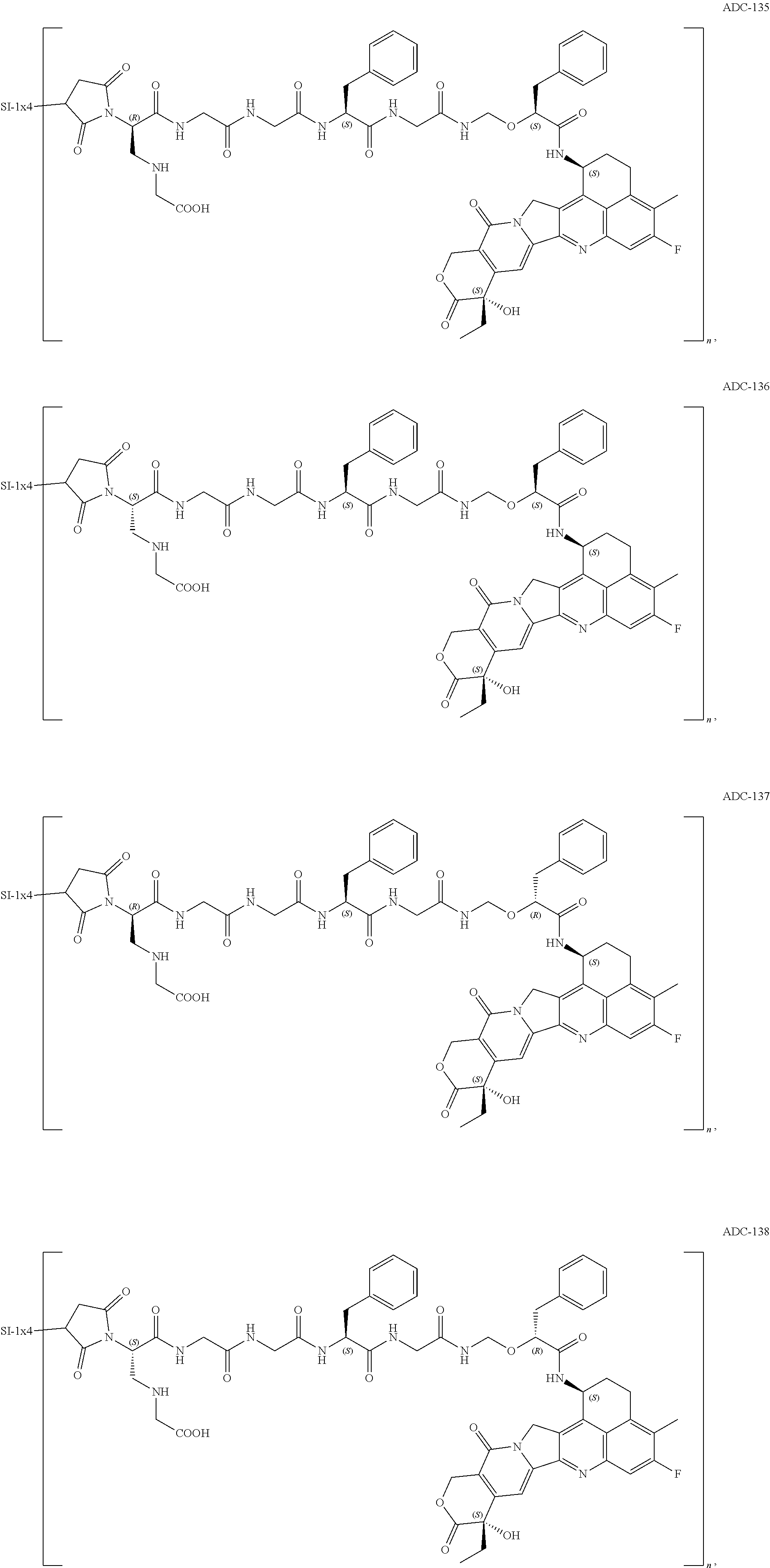

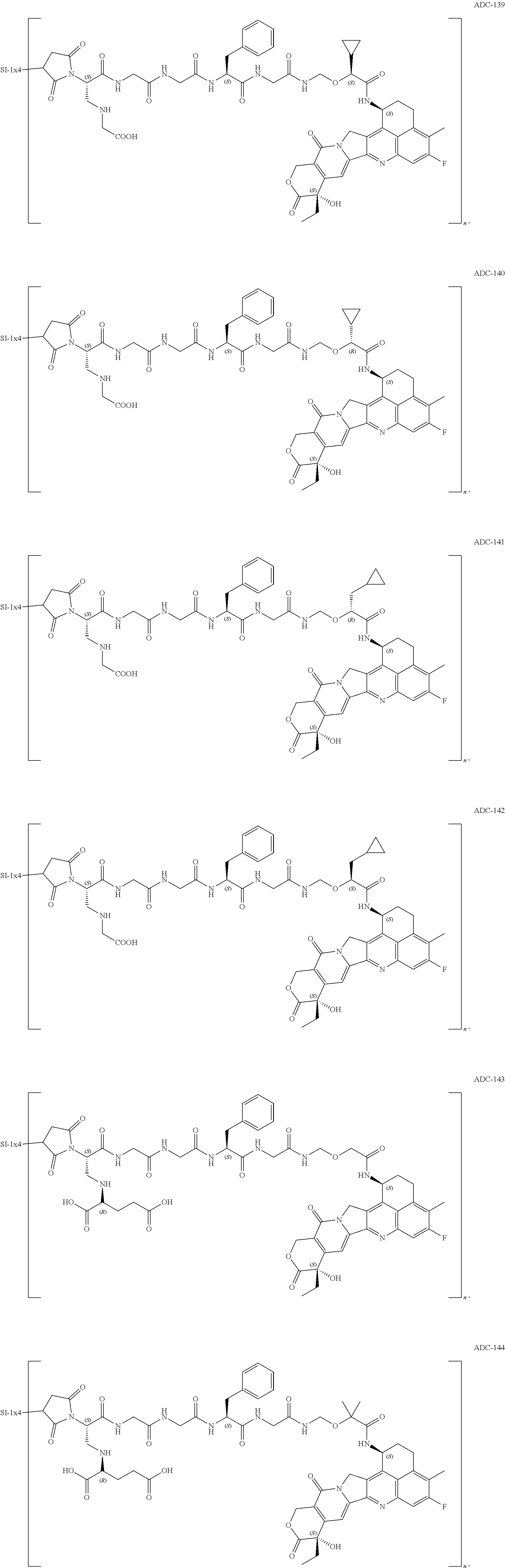

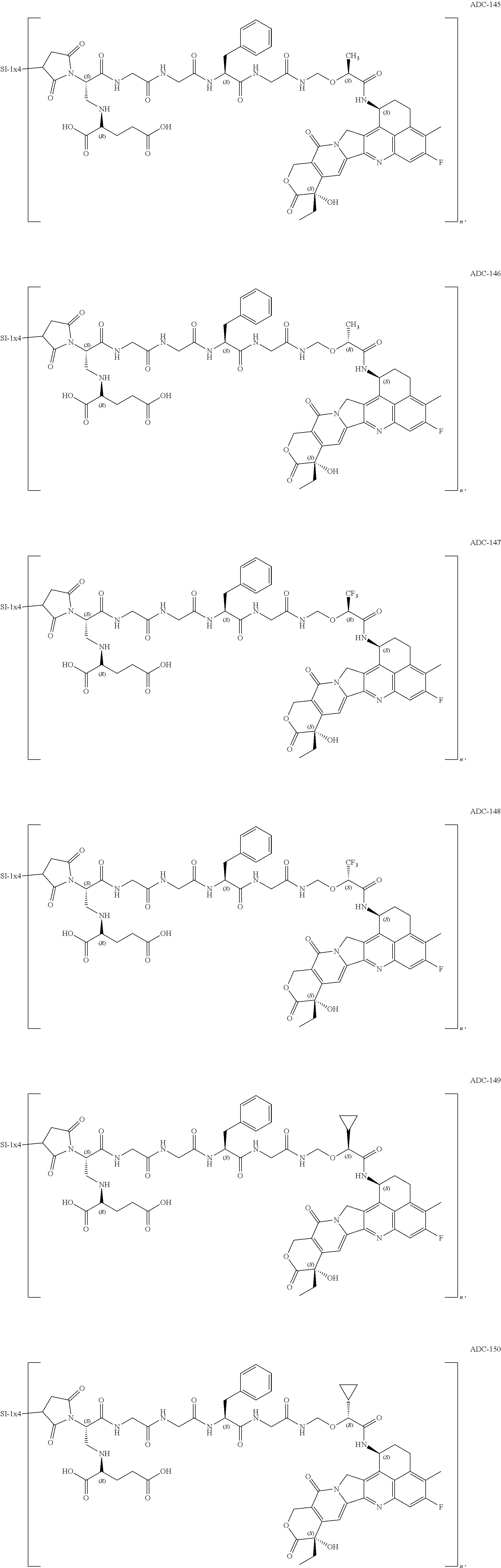

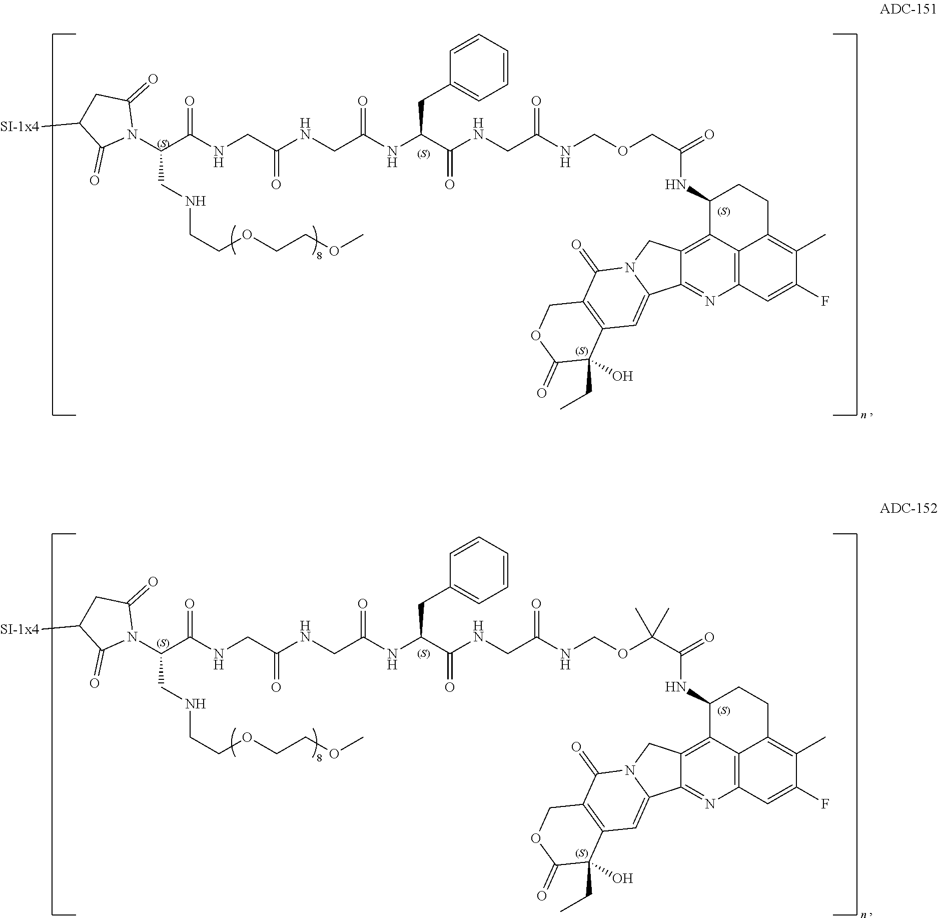

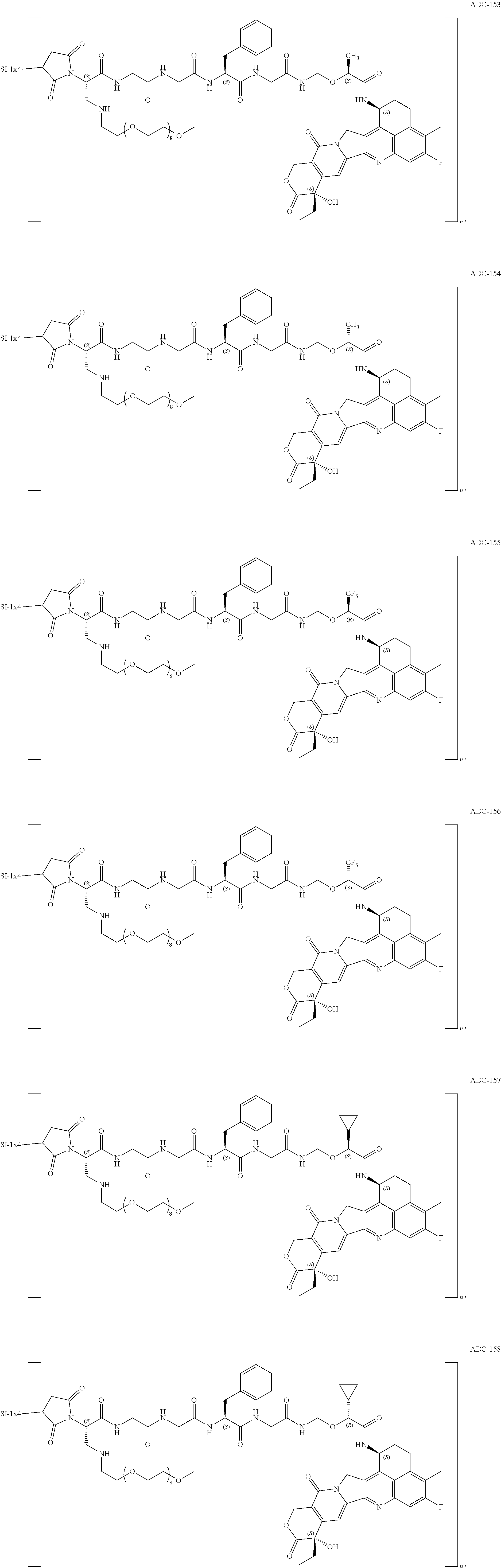

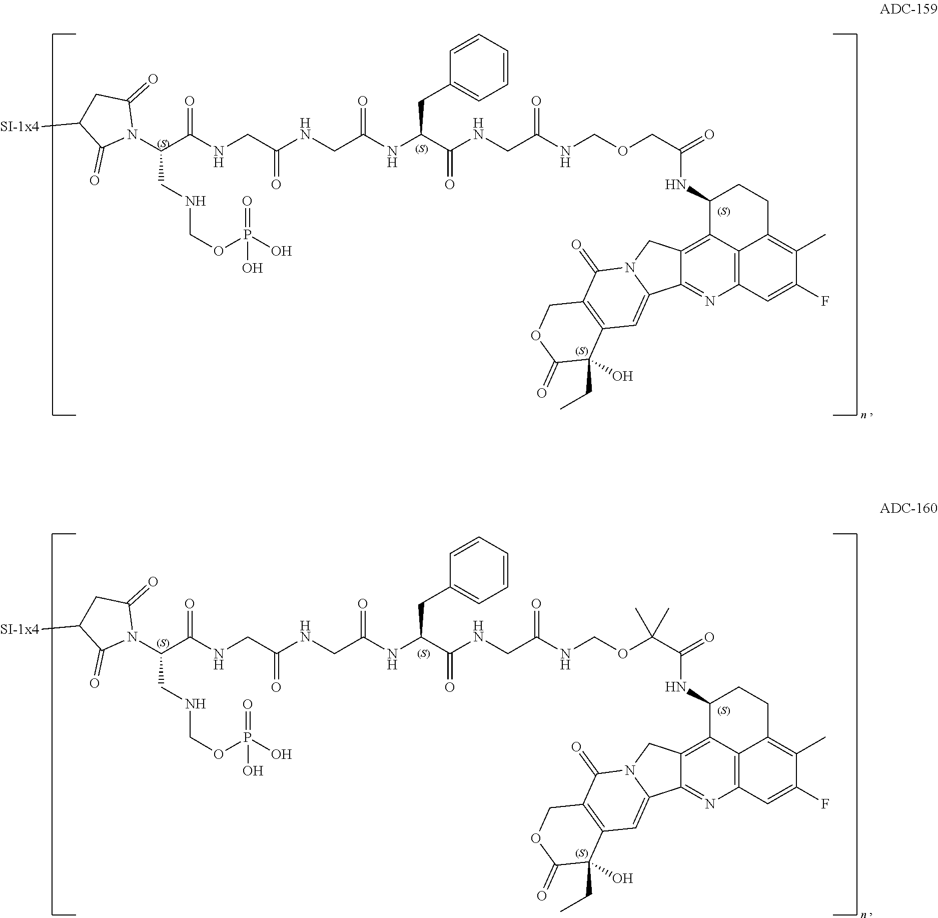

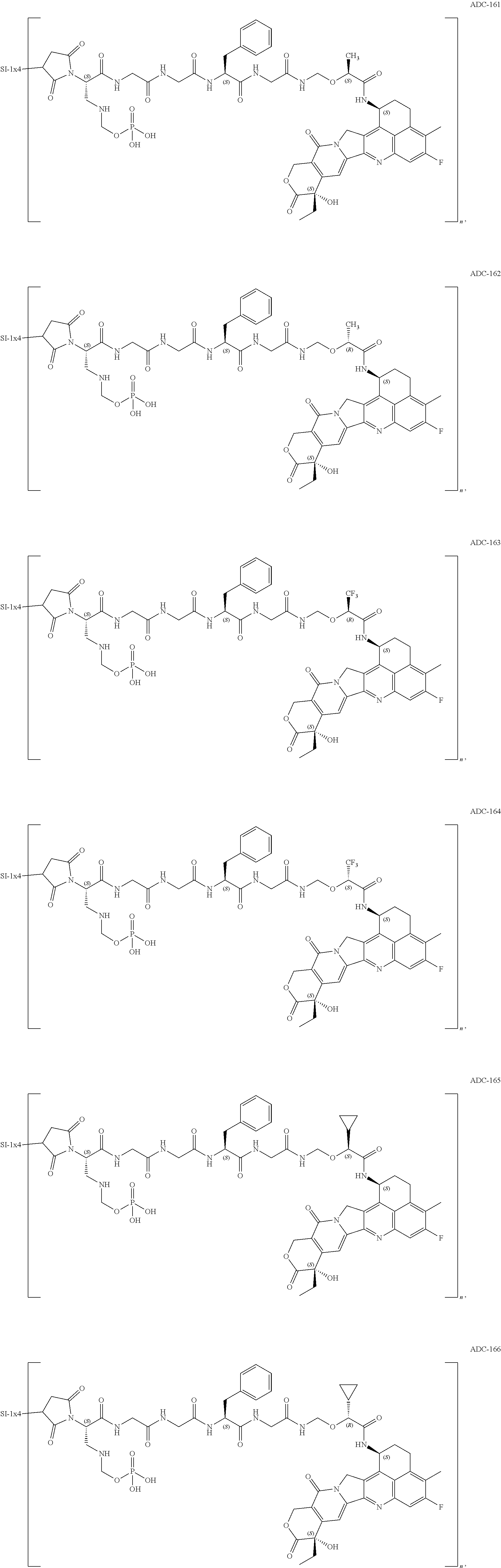

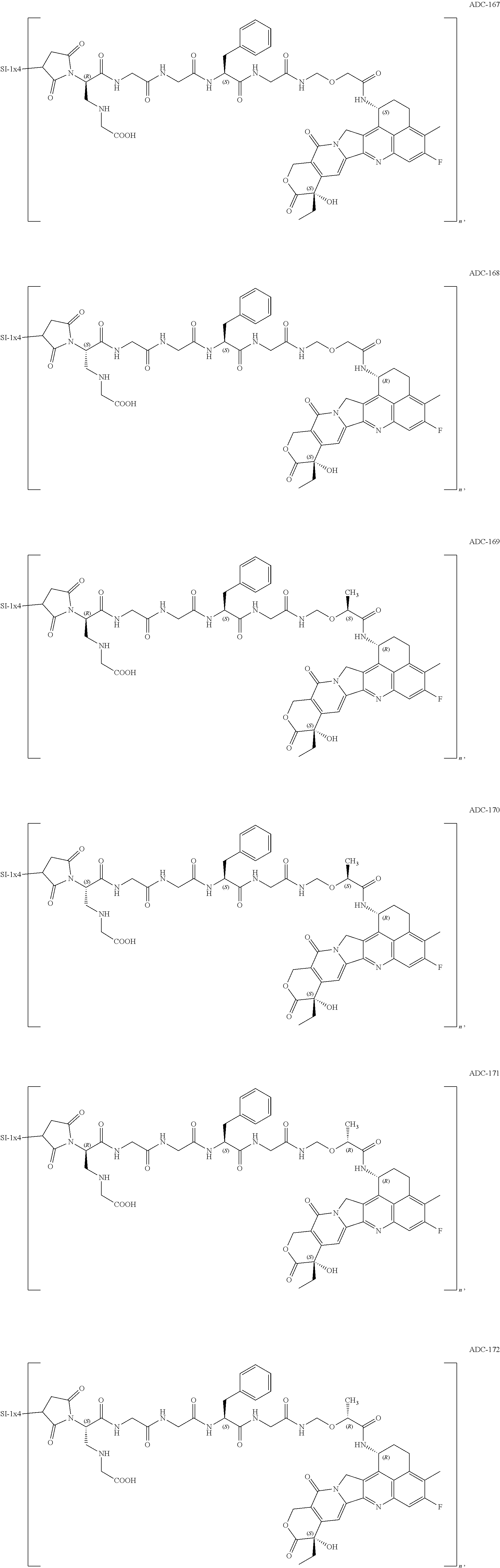

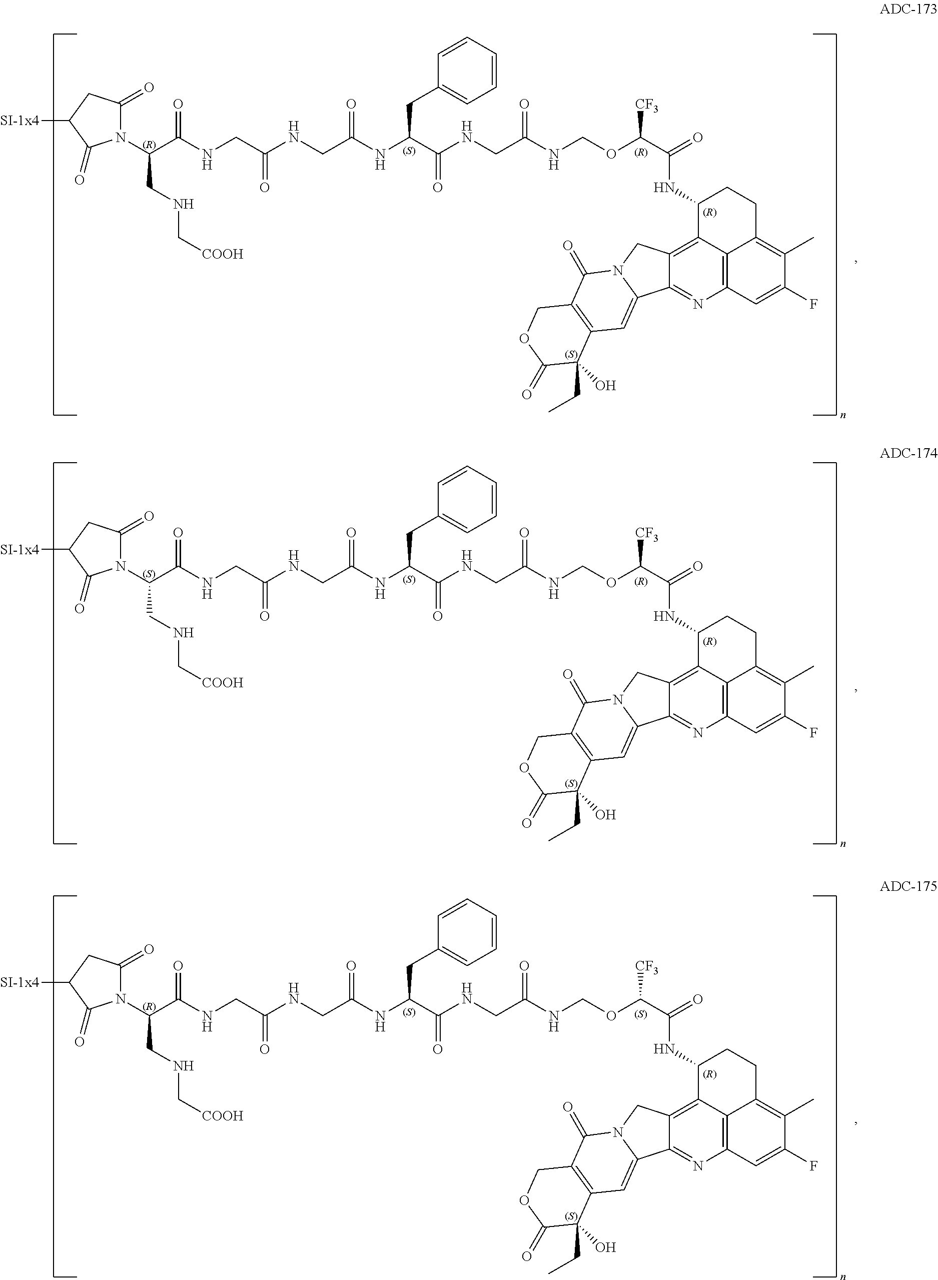

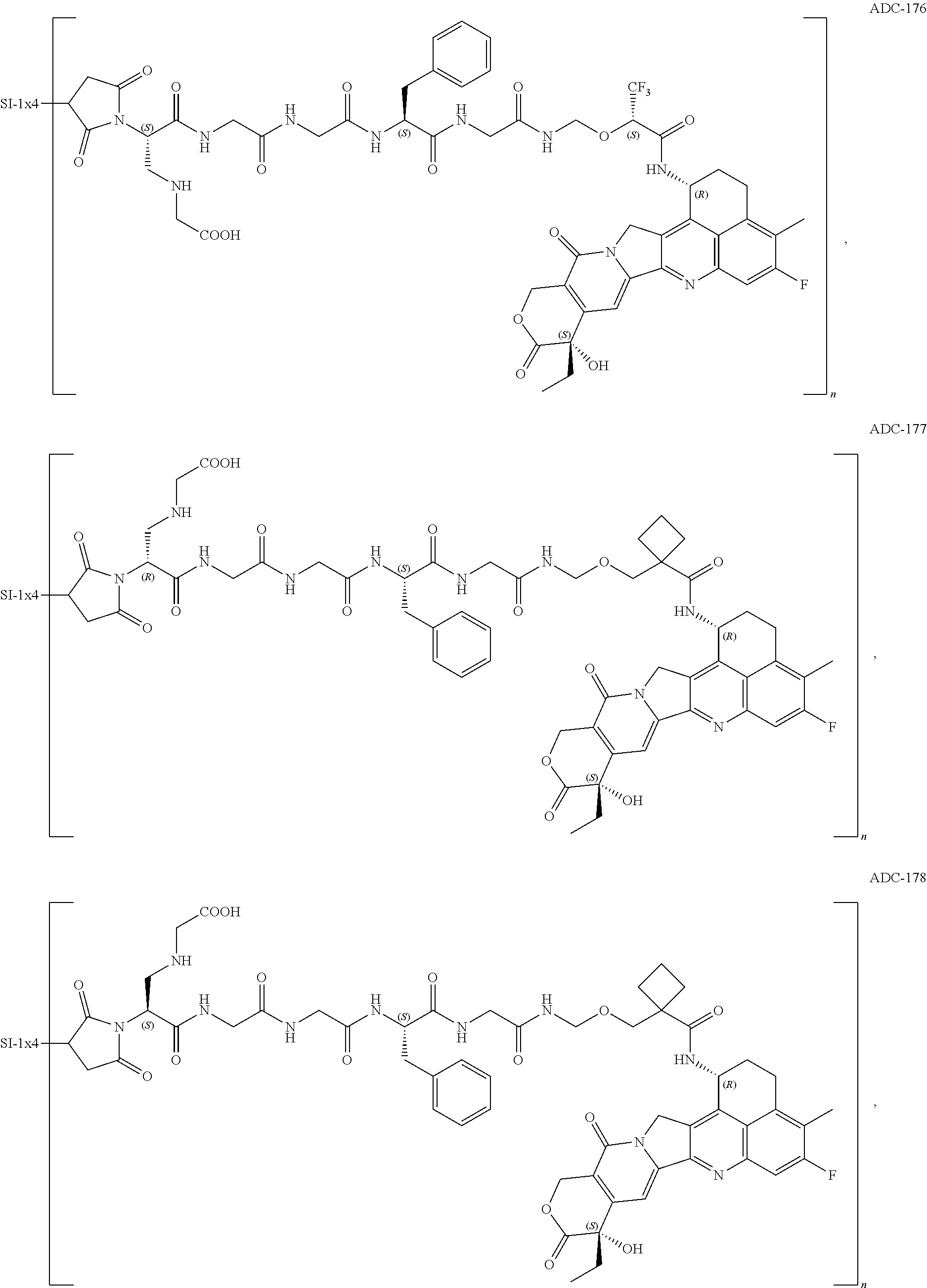

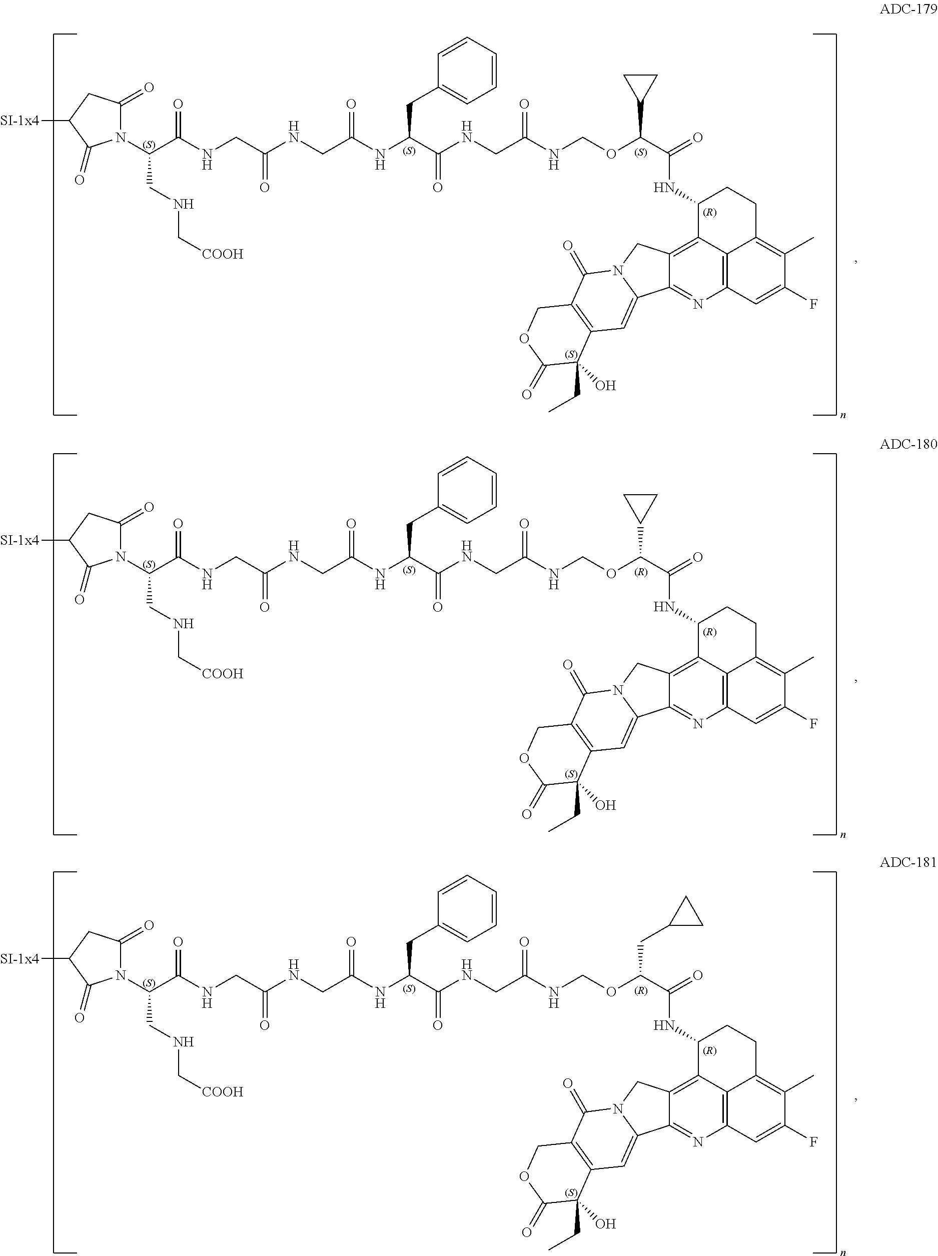

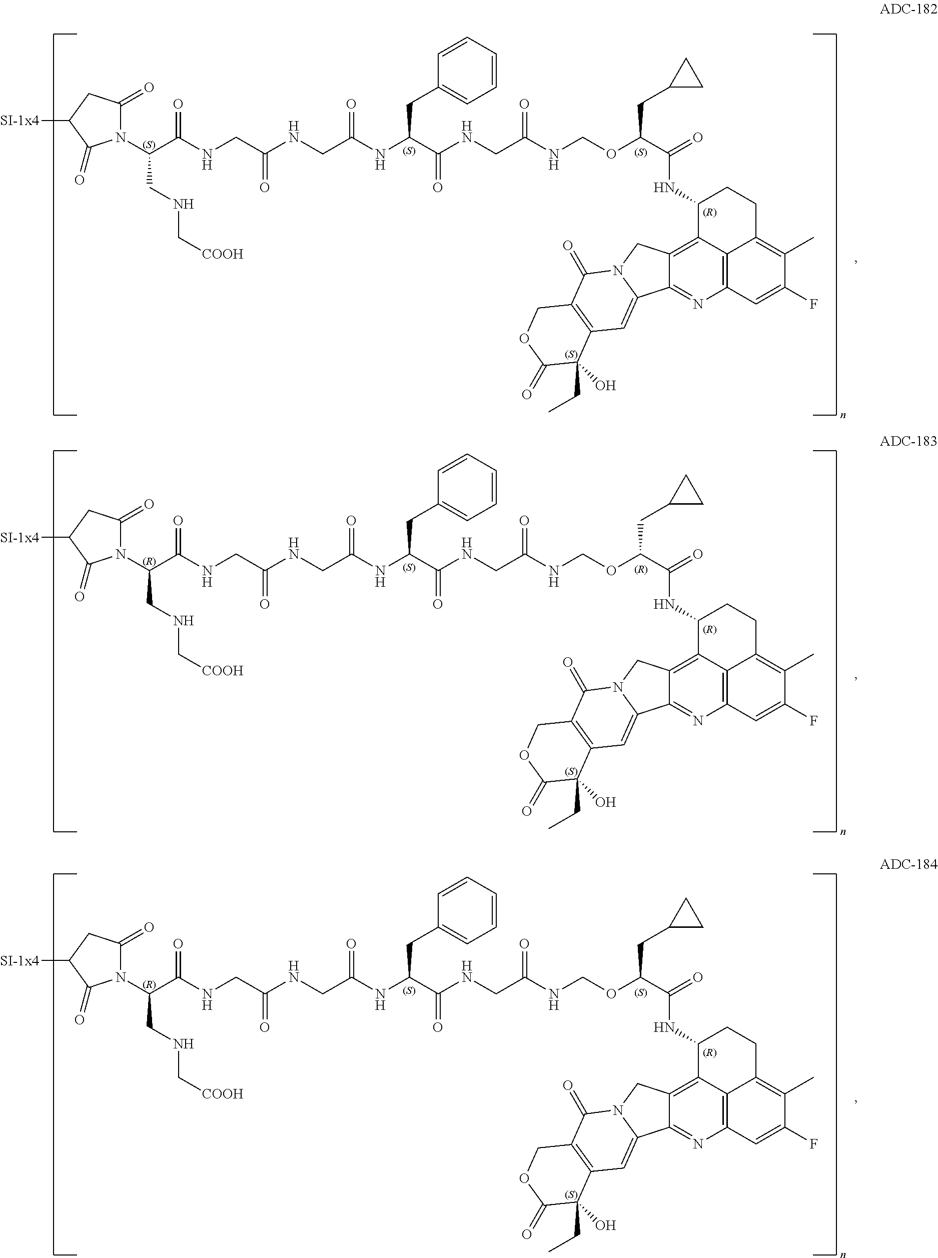

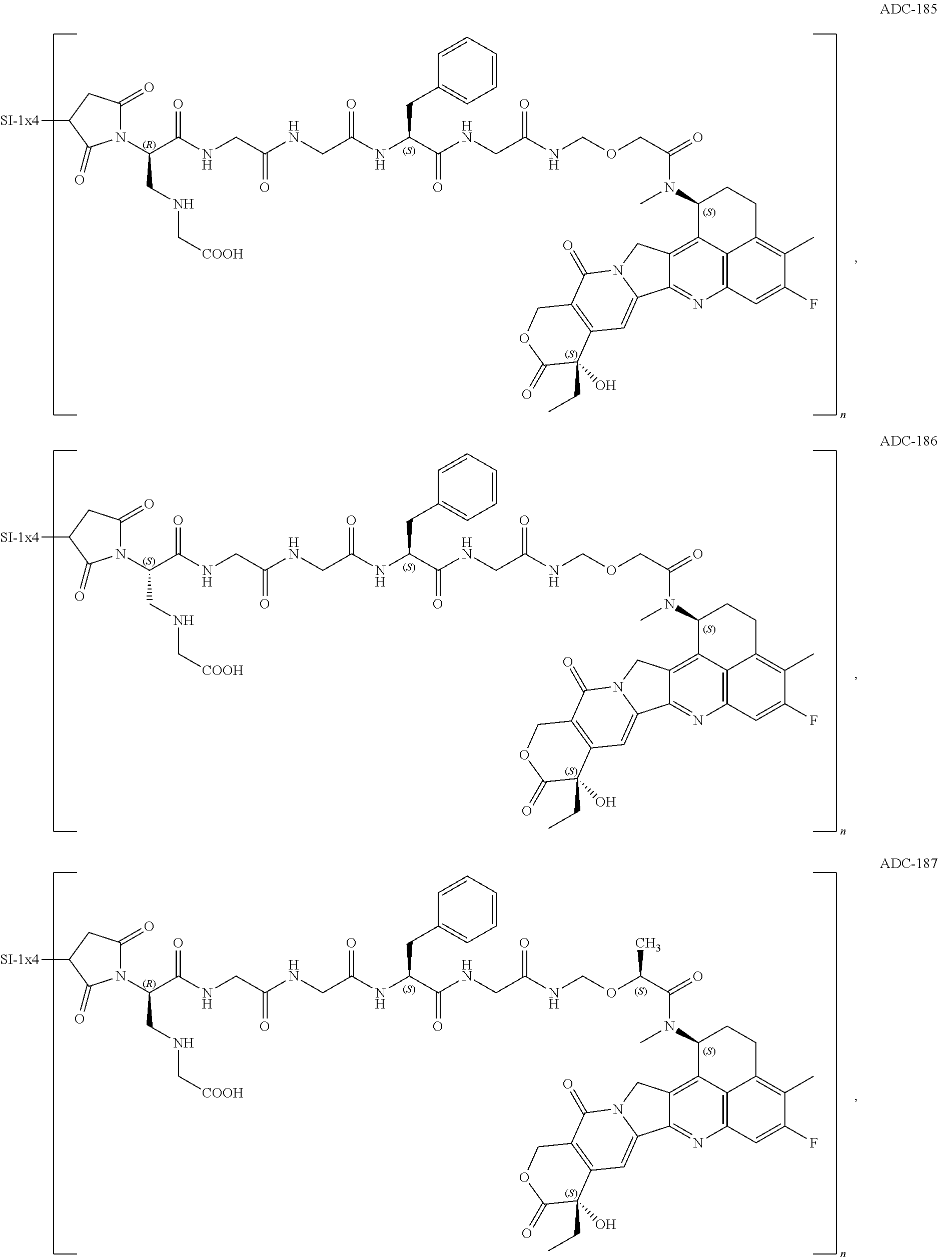

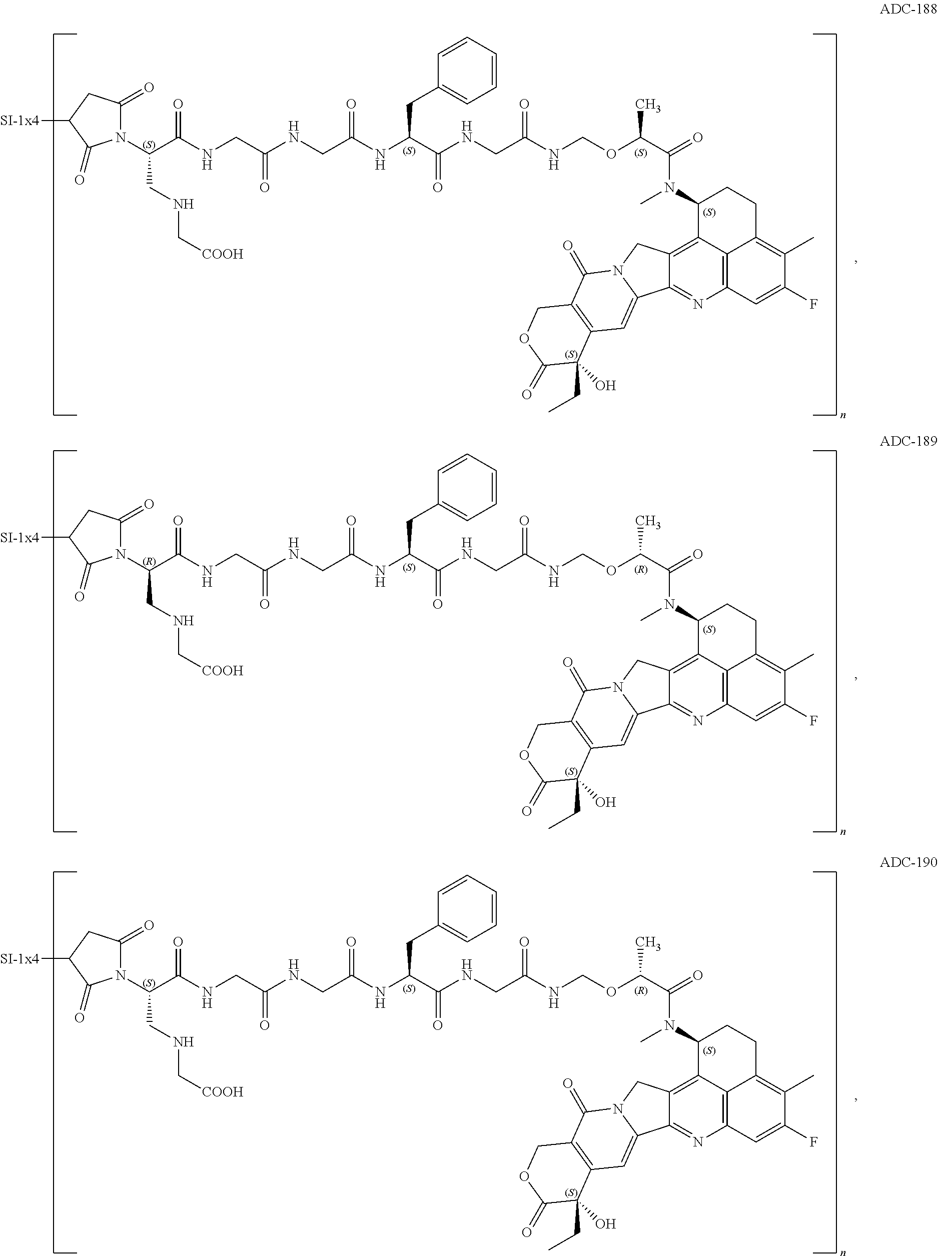

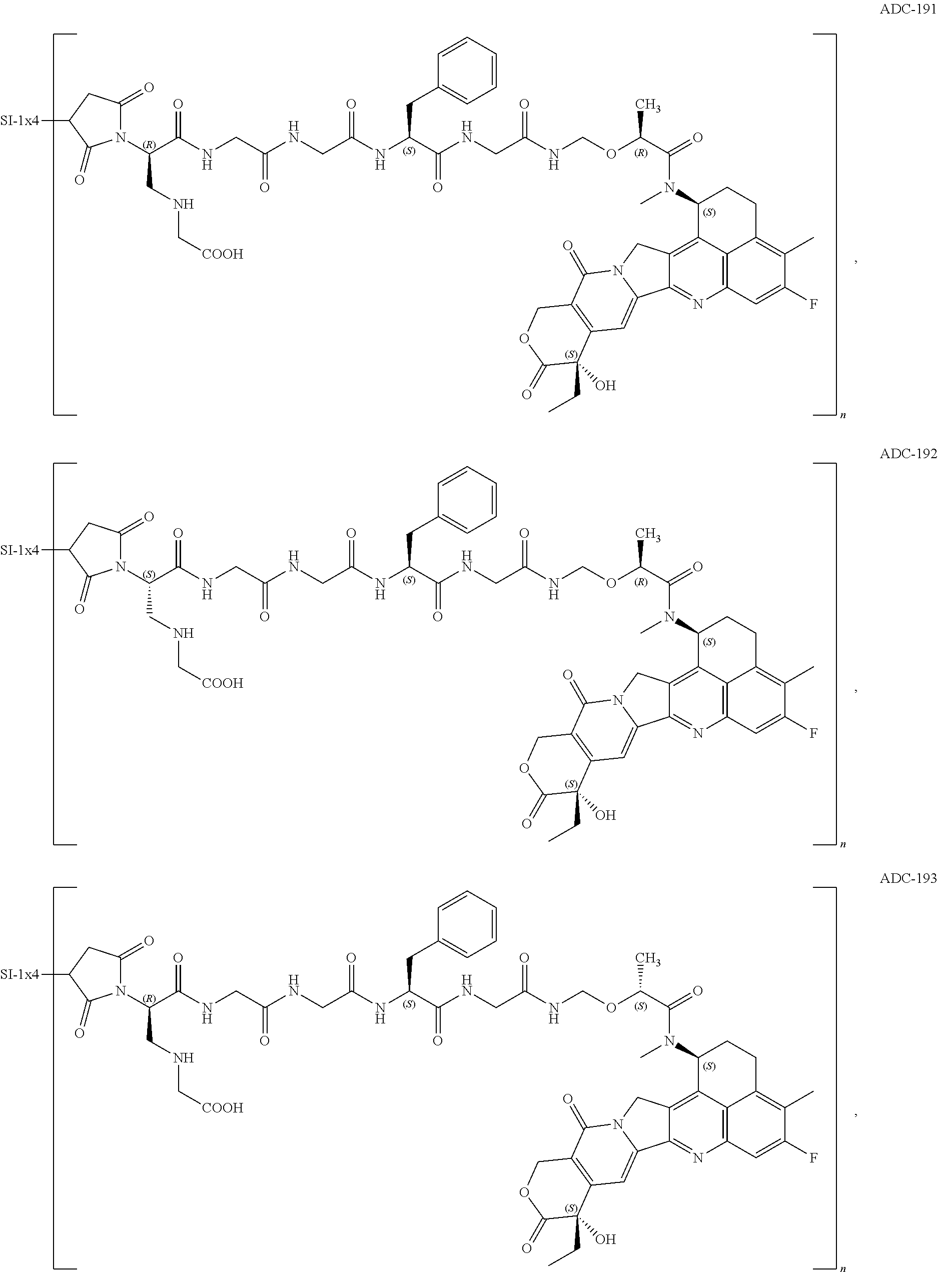

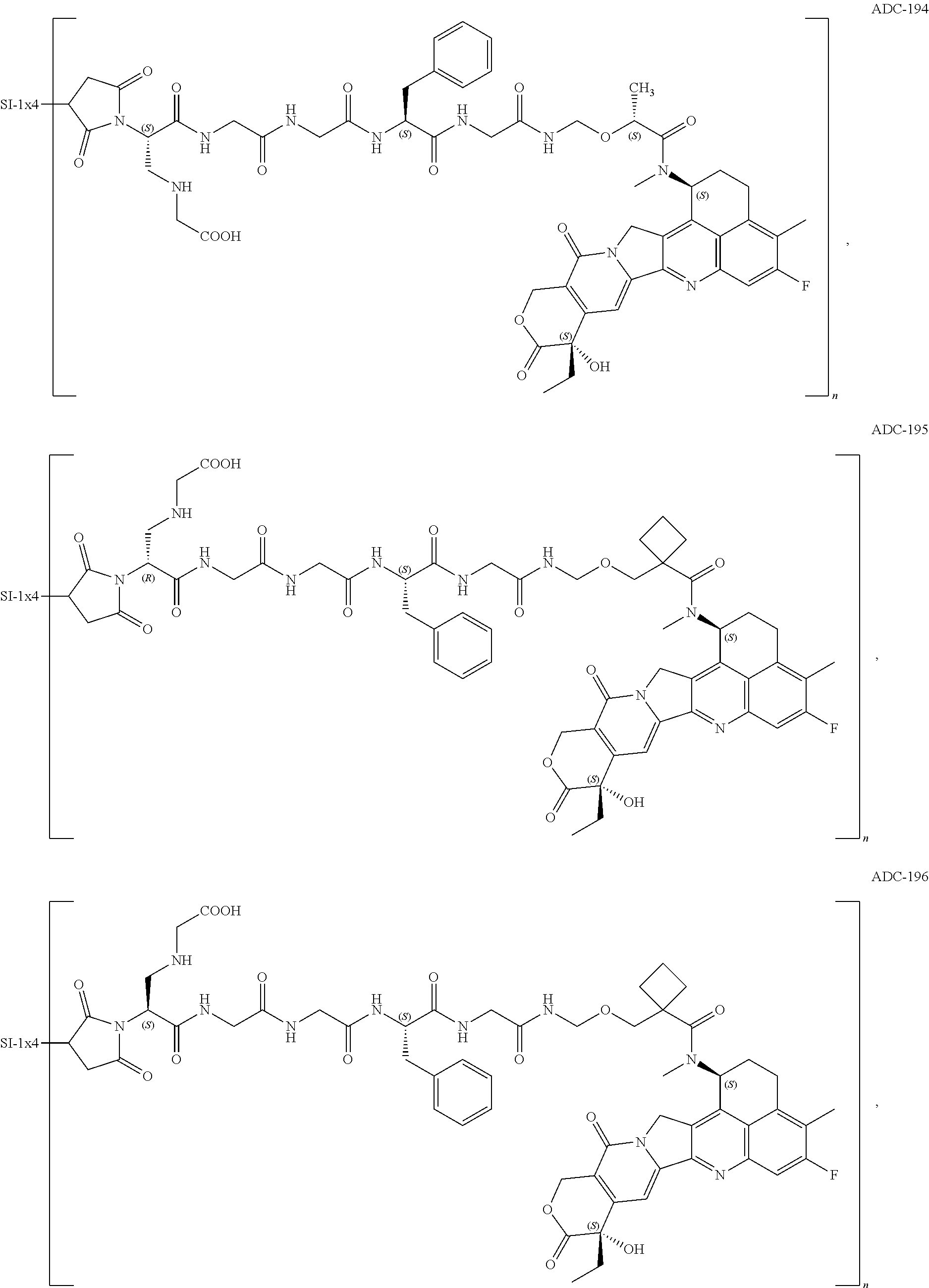

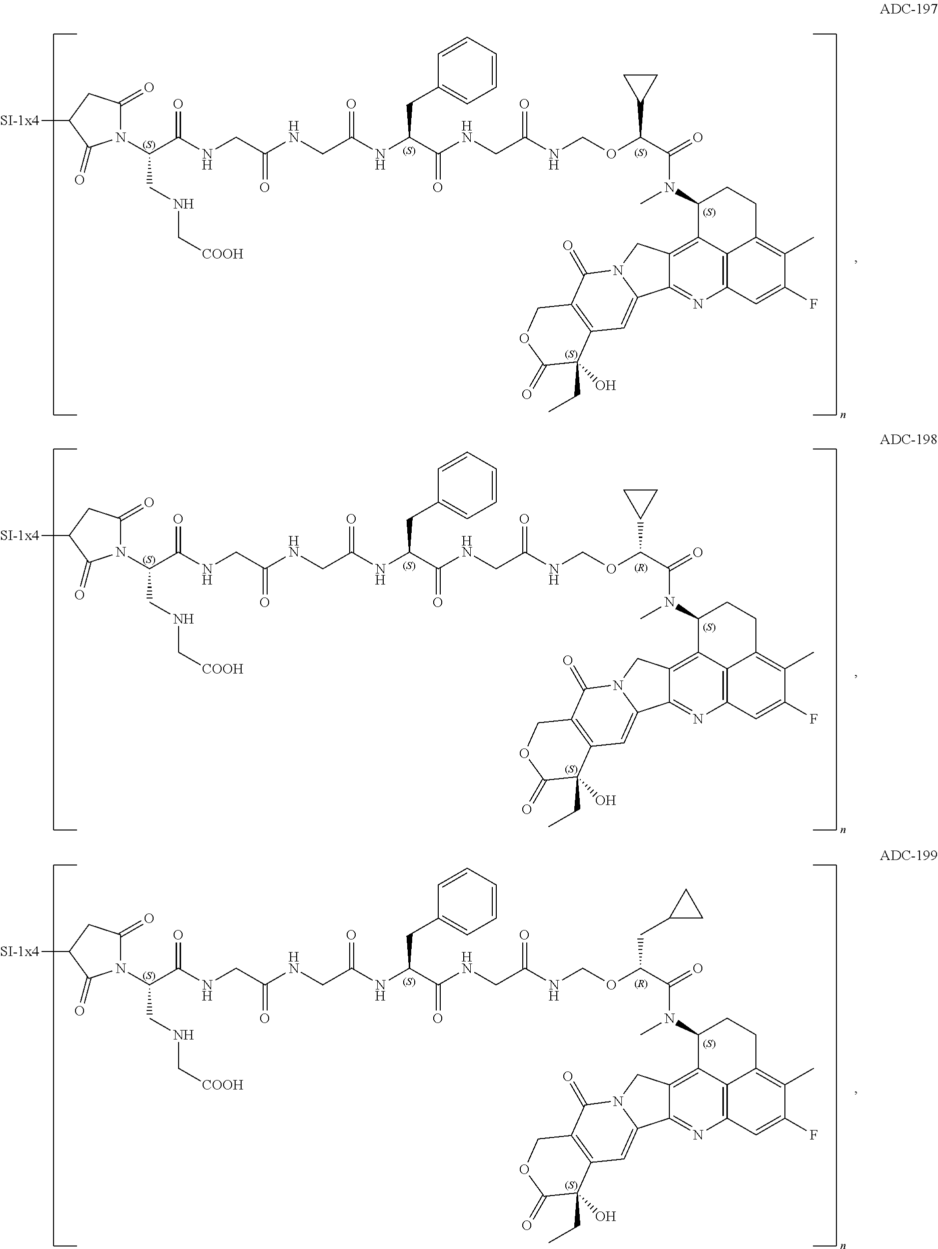

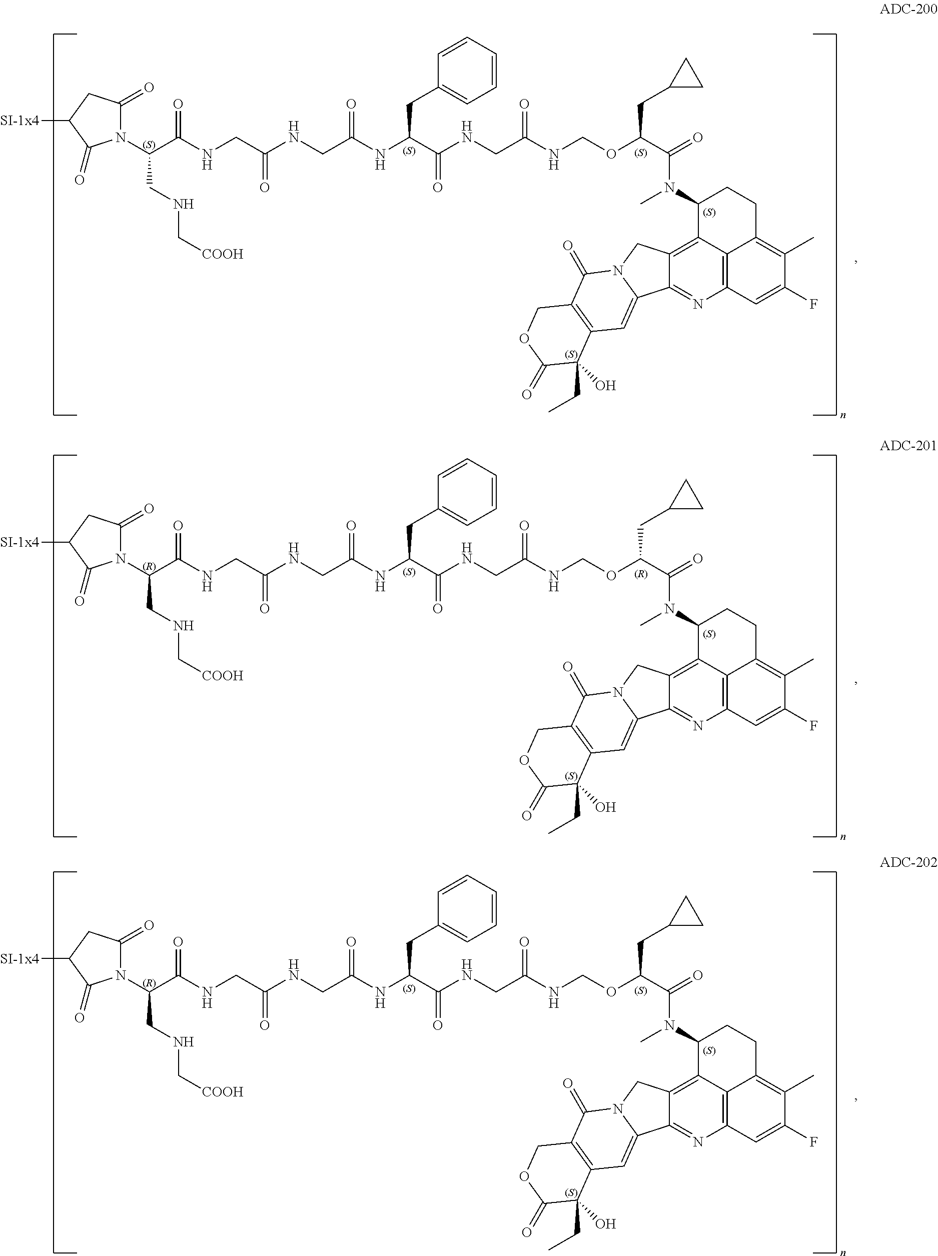

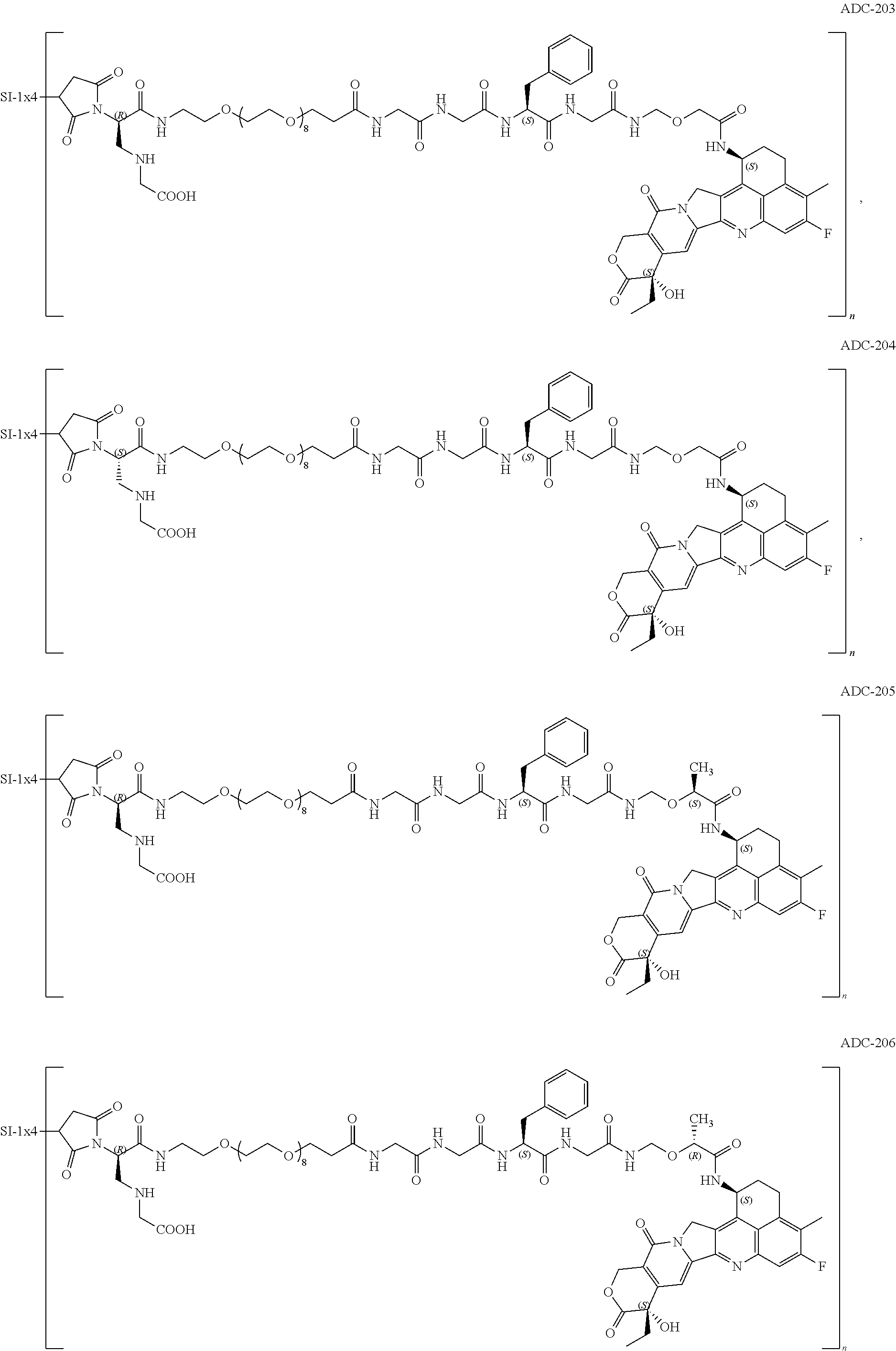

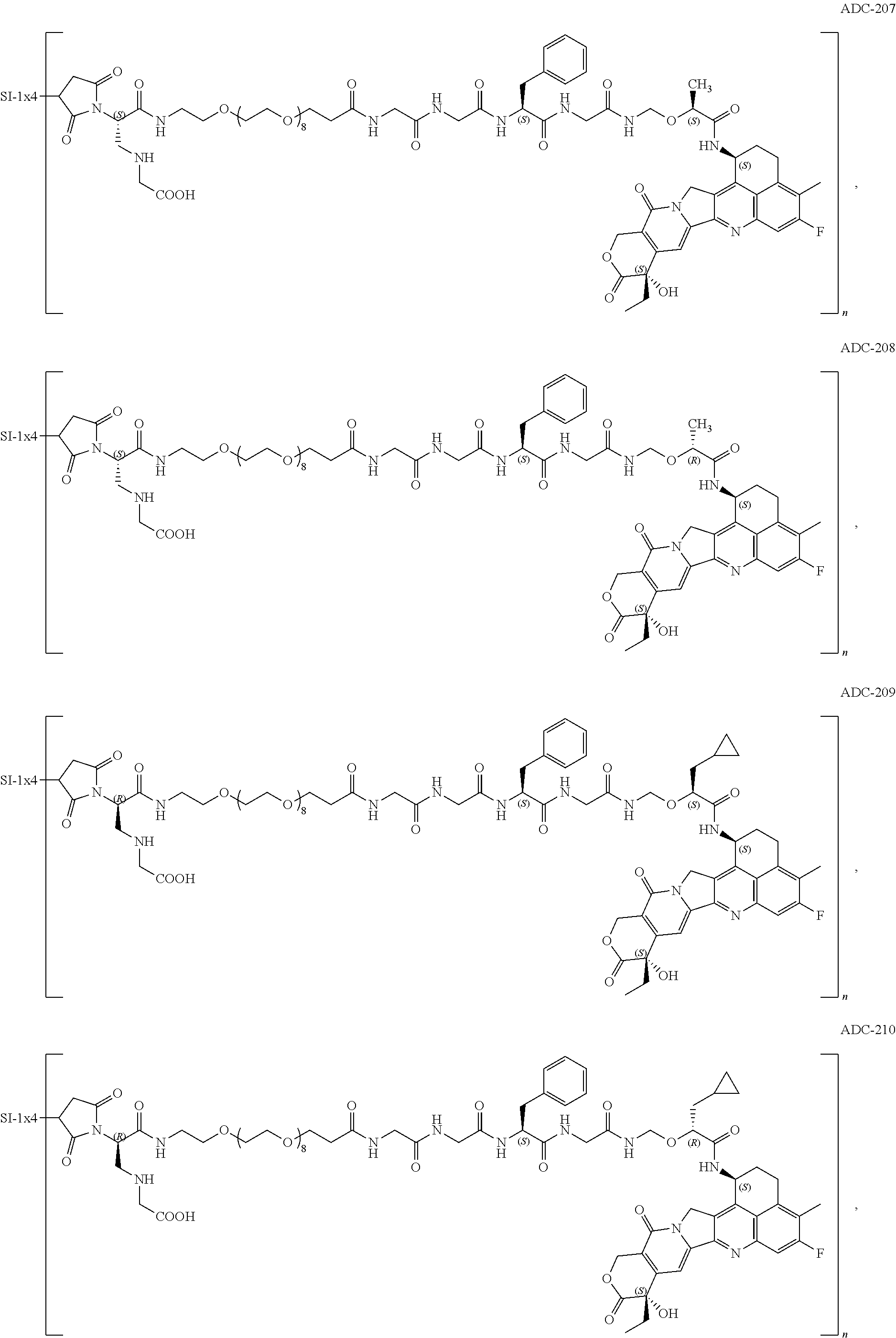

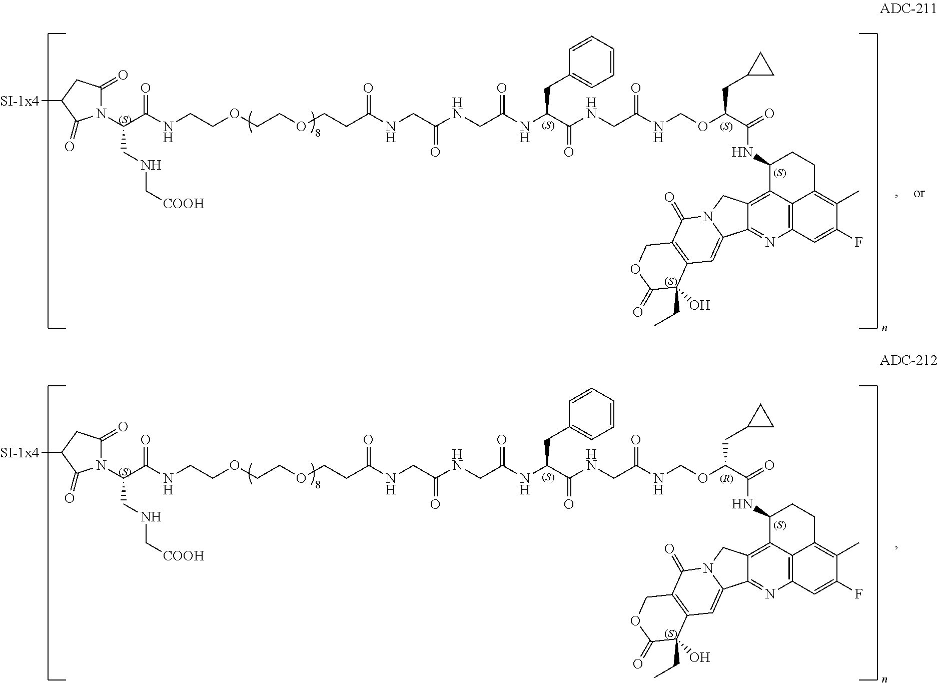

wherein:
SI-1×4 is a bispecific antibody or its antigen-binding fragment simultaneously targeting EGFR and HER3;
n is selected from the integers 1-10 (i.e., 1, 2, 3, 4, 5, 6, 7, 8, 9, or 10).

In some embodiments of the first aspect, the second aspect and the fourth aspect of the present invention, the disclosure of said ligand-camptothecin derivative conjugate, or a pharmaceutically acceptable salt or solvate thereof, is characterized in that said ligand-camptothecin derivative conjugate, or a pharmaceutically acceptable salt or solvate thereof, is selected without limitation from the following structures or succinimide open-ring structures thereof or isomers thereof,

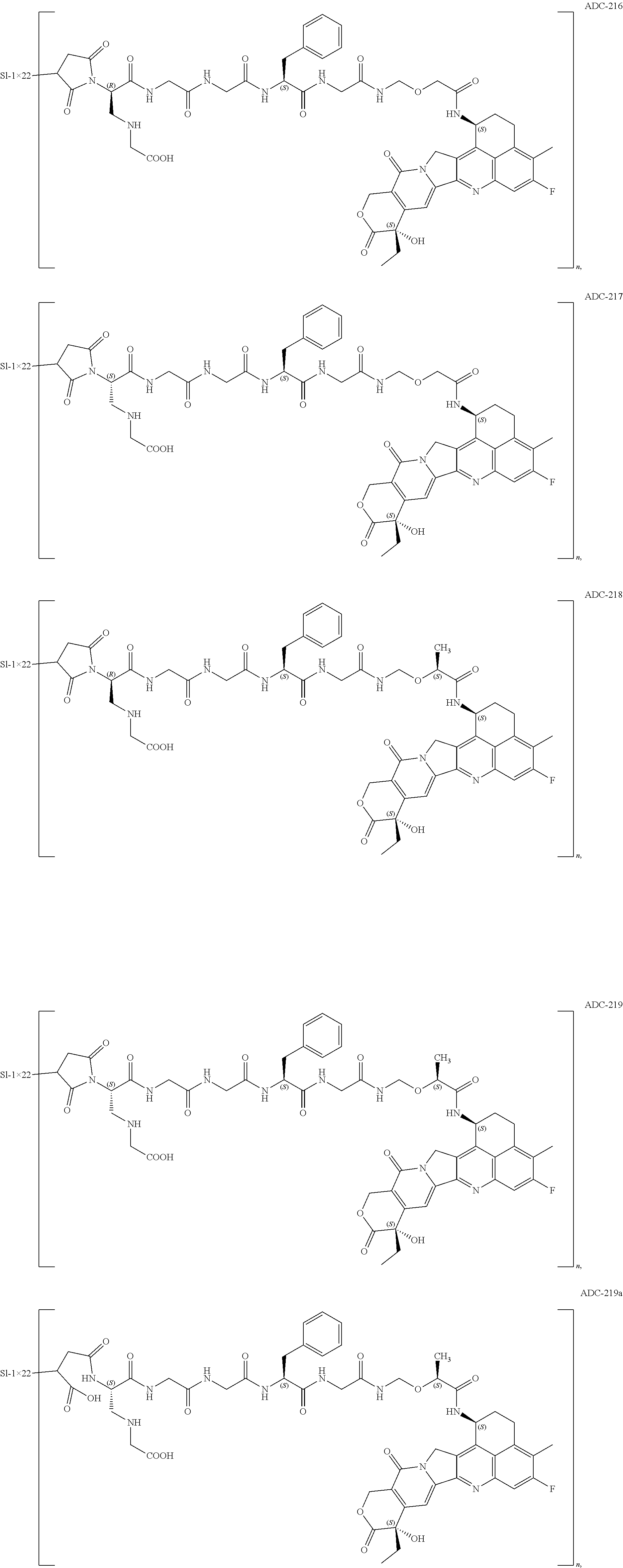

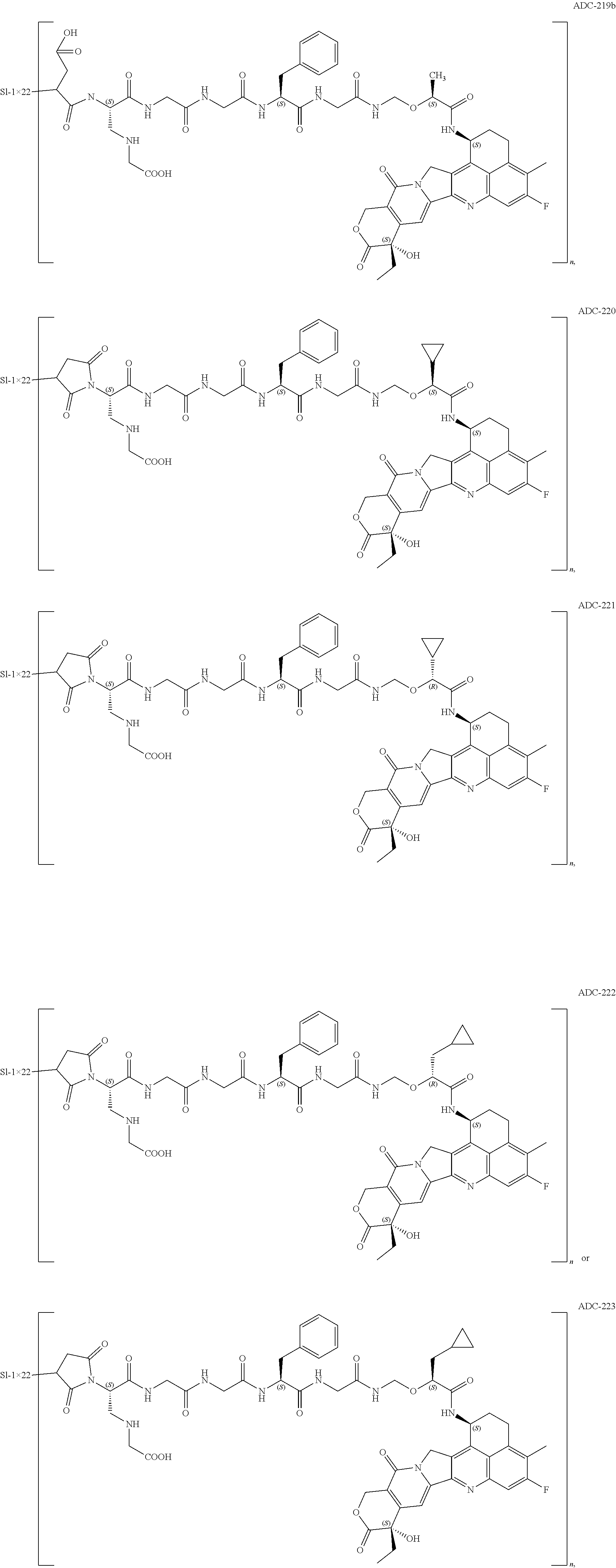

wherein:
SI-1×22 is a bispecific antibody or an antigen-binding fragment thereof simultaneously targeting EGFR and HER3;
n is selected from the integers 1-10 (e.g., 1, 2, 3, 4, 5, 6, 7, 8, 9, or 10).

In some embodiments of the first aspect, the second aspect and the fourth aspect of the present invention, the disclosure of said ligand-camptothecin derivative conjugate, or a pharmaceutically acceptable salt or solvate thereof, is characterized in that said ligand-camptothecin derivative conjugate, or a pharmaceutically acceptable salt or solvate thereof, is selected without limitation from the following structures or succinimide open-ring structures thereof or isomers thereof,

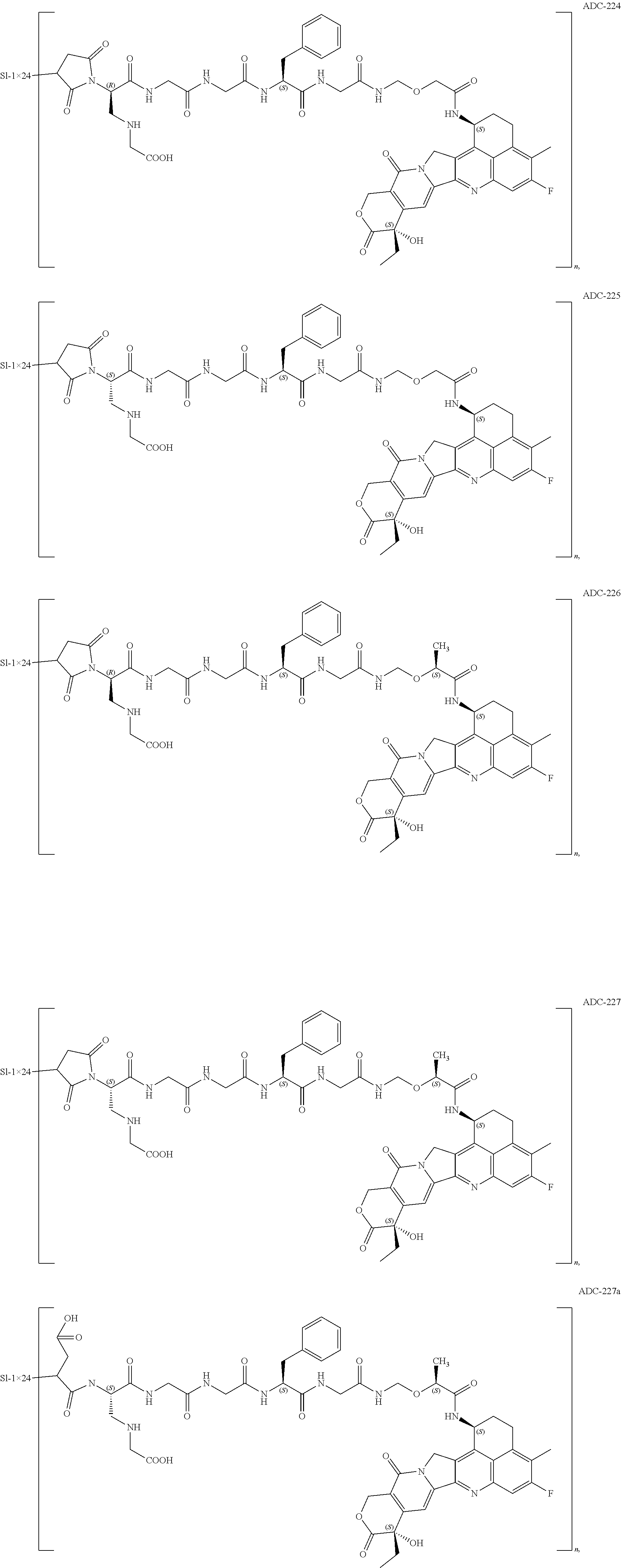

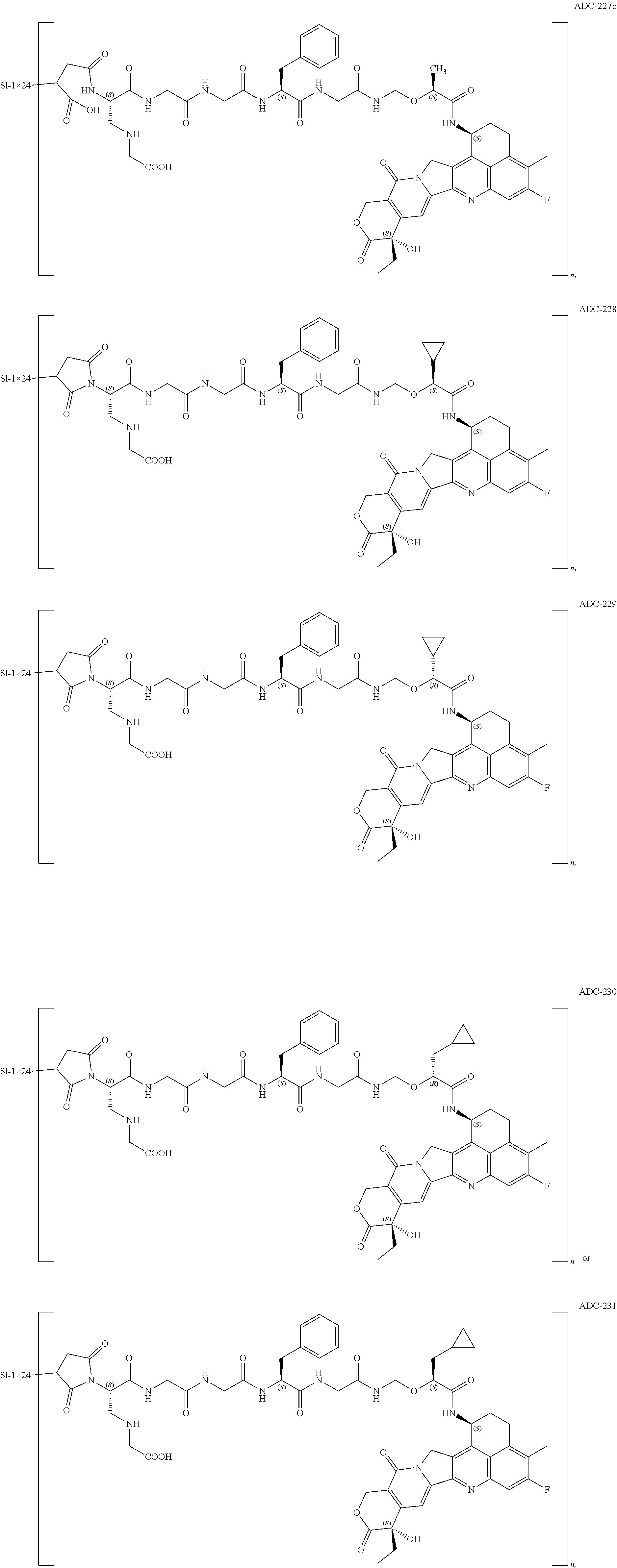

wherein:
SI-1×24 is a bispecific antibody or its antigen-binding fragment simultaneously targeting EGFR and HER3;
n is selected from the integers 1-10 (i.e., 1, 2, 3, 4, 5, 6, 7, 8, 9, or 10).

In some embodiments of the first aspect, the second aspect and the fourth aspect of the present invention, the disclosure of said ligand-camptothecin derivative conjugate, or a pharmaceutically acceptable salt or solvate thereof, is characterized in that said ligand-camptothecin derivative conjugate, or a pharmaceutically acceptable salt or solvate thereof, is selected without limitation from the following structures or succinimide open-ring structures thereof or isomers thereof,

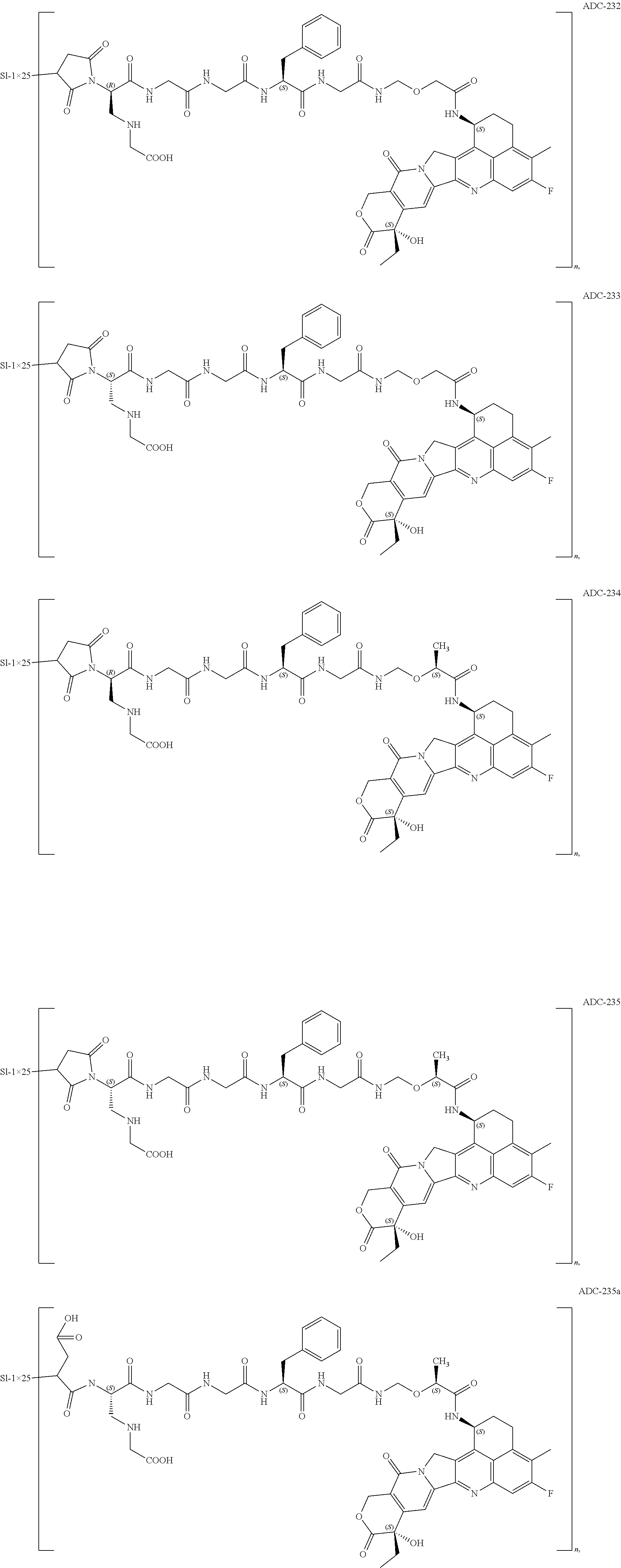

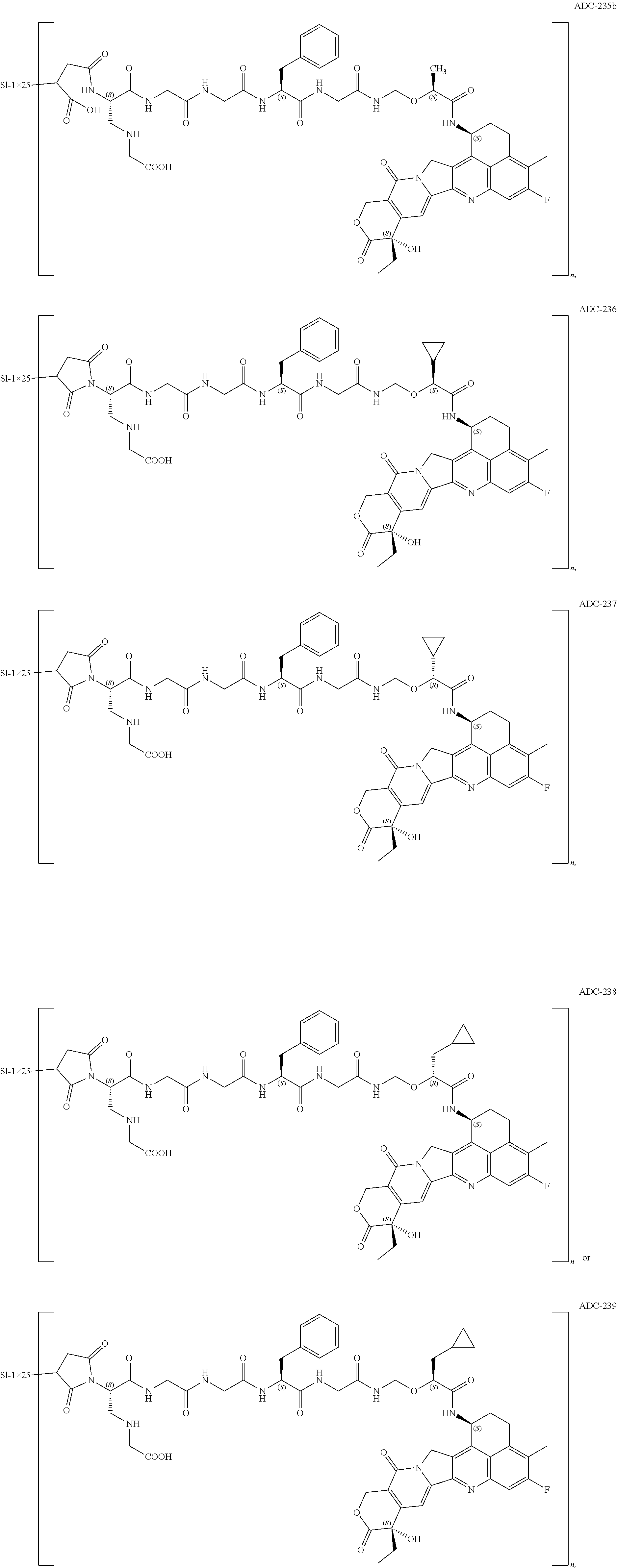

wherein:
SI-1×25 is a bispecific antibody or an antigen-binding fragment thereof simultaneously targeting EGFR and HER3;
n is selected from the integers 1-10 (i.e., 1, 2, 3, 4, 5, 6, 7, 8, 9, or 10).

In some embodiments of the first aspect, the second aspect and the fourth aspect of the present invention, the disclosure of said ligand-camptothecin derivative conjugate, or a pharmaceutically acceptable salt or solvate thereof, is characterized in that said ligand-camptothecin derivative conjugate, or a pharmaceutically acceptable salt or solvate thereof, is selected without limitation from the following structures or succinimide open-ring structures thereof or isomers thereof,

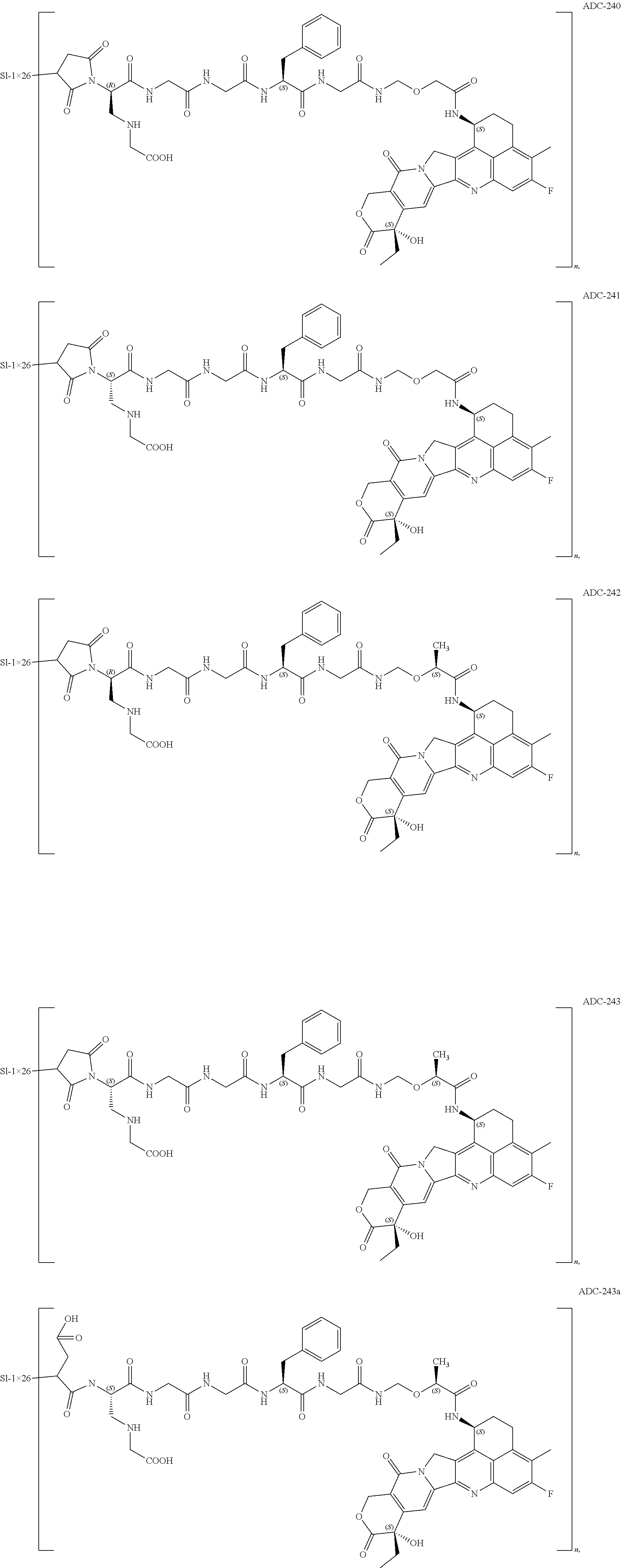

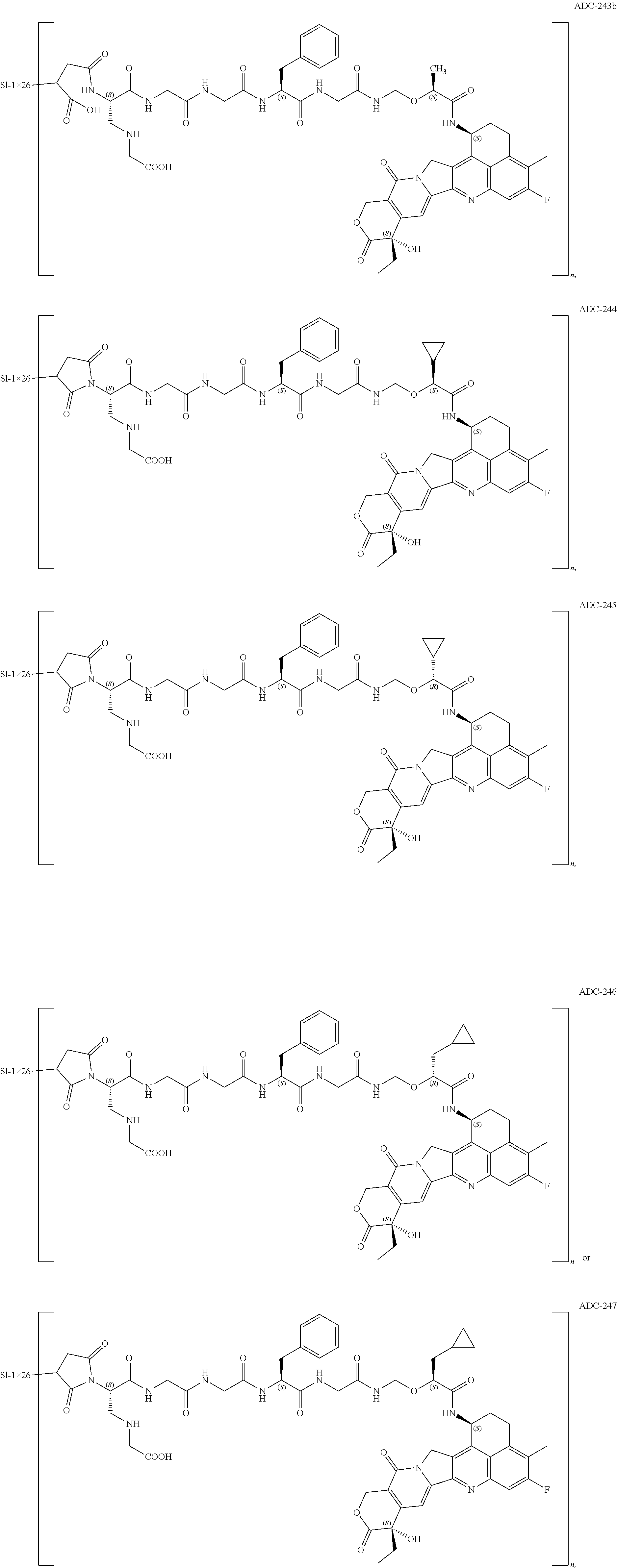

wherein:
SI-1×26 is a bispecific antibody or its antigen-binding fragment simultaneously targeting EGFR and HER3;
n is selected from the integers 1-10 (i.e., 1, 2, 3, 4, 5, 6, 7, 8, 9, or 10).

In some embodiments of the first aspect, the second aspect and the third aspect of the present invention, the disclosure of said ligand-camptothecin derivative conjugate or pharmaceutically acceptable salt or solvate thereof or linker-drug compound or pharmaceutically acceptable salt or solvate thereof is characterized in that said pharmaceutically acceptable salt comprises a sodium salt, a potassium salt, a calcium salt or a magnesium salt formed with an acidic functional group in the structural formula, and acetate, trifluoroacetate, citrate, oxalate, tartrate, malate, nitrate, chloride, bromide, iodide, sulfate, bisulfate, phosphate, lactate, oleate, ascorbate, salicylate, formate, glutamate, methanesulfonate, ethanesulfonate, benzenesulfonate or p-toluenesulfonate formed with a basic functional group in the structure.

A fifth aspect of the present invention discloses a pharmaceutical composition comprising a ligand-camptothecin derivative conjugate described in the first aspect and the second aspect or a pharmaceutically acceptable salt or solvate thereof or a linker-drug compound described in the third aspect or a pharmaceutically acceptable salt or solvate thereof, and optionally a pharmaceutically acceptable carrier.

A sixth aspect of the present invention discloses a pharmaceutical preparation comprising a ligand-camptothecin derivative conjugate described in the first aspect and the second aspect, or a pharmaceutically acceptable salt or solvate thereof, or a linker-drug compound described in the third aspect, or a pharmaceutically acceptable salt or solvate thereof.

A seventh aspect of the present invention discloses the use of the following in the preparation of drugs for the treatment or prevention of cancers or tumors: the ligand-camptothecin derivative conjugates described in the first aspect and the second aspect or pharmaceutically acceptable salts or solvates thereof, or the linker-drug compounds described in the third aspect or pharmaceutically acceptable salts or solvates thereof, or the pharmaceutical compositions described in the fifth aspect and/or the pharmaceutical preparations described in the sixth aspect;
alternatively, the use of the following in the treatment or prevention of cancer or tumors: the ligand-camptothecin derivative conjugates described in the first aspect and the second aspect or pharmaceutically acceptable salts or solvates thereof, or the linker-drug compounds described in the third aspect or pharmaceutically acceptable salts or solvates thereof, or the pharmaceutical compositions described in the fifth aspect and/or the pharmaceutical preparations described in the sixth aspect;
preferably, the cancer or tumor expresses EGFR and/or HER3;
more preferably, the cancer or tumor is selected from adenocarcinoma, ovarian cancer, cervical cancer, uterine cancer, prostate cancer, renal cancer, urethral cancer, bladder cancer, liver cancer, gastric cancer, endometrial cancer, salivary gland cancer, esophageal cancer, lung cancer, colon cancer, rectal cancer, colorectal cancer, bone cancer, skin cancer, thyroid cancer, pancreatic cancer, melanoma, glioma, neuroblastoma, glioblastoma multiforme, sarcoma, lymphoma and leukemia, and other solid tumors or blood tumors.

An eighth aspect of the present invention discloses a method of treating or preventing cancer or tumors, the method comprising administering to a subject in need thereof a prophylactically or therapeutically effective amount of a ligand-camptothecin derivative conjugate described in the first or second aspect or a pharmaceutically acceptable salt or solvate thereof or a linker-drug compound described in the third aspect or a pharmaceutically acceptable salt or solvate thereof, or a pharmaceutical composition as described in the fifth aspect and/or a pharmaceutical preparation described in the sixth aspect;
preferably, the cancer or tumor expresses EGFR and/or HER3;
more preferably, the cancer or tumor is selected from adenocarcinoma, ovarian cancer, cervical cancer, uterine cancer, prostate cancer, renal cancer, urethral cancer, bladder cancer, liver cancer, gastric cancer, endometrial cancer, salivary gland cancer, esophageal cancer, lung cancer, colon cancer, rectal cancer, colorectal cancer, bone cancer, skin cancer, thyroid cancer, pancreatic cancer, melanoma, glioma, neuroblastoma, glioblastoma multiforme, sarcoma, lymphoma and leukemia, and other solid tumors or blood tumors.

In the above aspects of the present invention and its embodiments,
“C1-C6 alkyl” and “C1-C6 alkyl” in various composite groups involving “C1-C6 alkyl” (e.g., “substituted C1-C6 alkyl”, “deuterated C1-C6 alkyl”) may be replaced with “C1-C20 alkyl”, “C1-C12 alkyl” or “C1-C10 alkyl”;
“C3-C8 cycloalkyl” and “C3-C8 cycloalkyl” in various composite groups involving “C3-C8 cycloalkyl” may be replaced by “C3-C20 cycloalkyl” or “C3-C10 cycloalkyl”;
“C1-C6 alkoxy” and “C1-C6 alkoxy” in various composite groups involving same can be replaced with “C1-C20 alkoxy”, “C1-C12 alkoxy” or “C1-C10 alkoxy”;
“C6-C10 aryl” and “C6-C10 aryl” in various composite groups involving same can be replaced with “C6-C12 aryl”;
“3-7-membered heterocyclic group” and “3-7-membered heterocyclic group” in various composite groups involving same may be replaced with “3-20-membered heterocyclic group”, “3-12-membered heterocyclic group” or “3-10-membered heterocyclic group”.

### Beneficial Effects

The EGFR/HER3 bispecific antibody-drug conjugate provided by the present invention is a bispecific antibody ADC directed against both EGFR/HER3 targets, with good molecular stability and good preclinical efficacy, and is expected to have excellent clinical therapeutic effects.

### DESCRIPTION OF DRAWINGS

FIG. 1A illustrates SEC-HPLC detection/measurement of ADC-5 aggregation.

FIG. 1B illustrates SEC-HPLC detection/measurement of ADC-6 aggregation.

FIG. 1C illustrates SEC-HPLC detection/measurement of ADC-64 aggregation.

FIG. 1D illustrates SEC-HPLC detection/measurement of ADC-DS aggregation.

FIG. 1E illustrates SEC-HPLC detection/measurement of ADC-108 aggregation.

FIG. 1F illustrates SEC-HPLC detection/measurement of ADC-112 aggregation.

FIG. 1G illustrates SEC-HPLC detection/measurement of ADC-215 aggregation.

FIG. 1H illustrates SEC-HPLC detection/measurement of ADC-219 aggregation.

FIG. 1I illustrates SEC-HPLC detection/measurement of ADC-227 aggregation.

FIG. 1J illustrates SEC-HPLC detection/measurement of ADC-235 aggregation.

FIG. 2A illustrates RP-HPLC detection/measurement of ADC-5 drug-antibody coupling ratio (DAR).

FIG. 2B illustrates RP-HPLC detection/measurement of ADC-6 drug-antibody coupling ratio (DAR).

FIG. 2C illustrates RP-HPLC detection/measurement of ADC-10 drug-antibody coupling ratio (DAR).

FIG. 2D illustrates RP-HPLC detection/measurement of ADC-12 drug-antibody coupling ratio (DAR).

FIG. 2E illustrates RP-HPLC detection/measurement of ADC-64 drug-antibody coupling ratio (DAR).

FIG. 2F illustrates RP-HPLC detection/measurement of ADC-108 drug-antibody coupling ratio (DAR).

FIG. 2G illustrates RP-HPLC detection/measurement of ADC-112 drug-antibody coupling ratio (DAR).

FIG. 2H illustrates RP-HPLC detection/measurement of ADC-215 drug-antibody coupling ratio (DAR).

FIG. 2I illustrates RP-HPLC detection/measurement of ADC-219 drug-antibody coupling ratio (DAR).

FIG. 2J illustrates RP-HPLC detection/measurement of ADC-227 drug-antibody coupling ratio (DAR).

FIG. 2K illustrates RP-HPLC detection/measurement of ADC-235 drug-antibody coupling ratio (DAR).

FIG. 2L illustrates RP-HPLC detection/measurement of ADC-243 drug-antibody coupling ratio (DAR).

FIG. 3A illustrates that ADC-112 and the SI-1×4 antibody maintain affinity for both the antigens EGFR and HER3-Fc.

FIG. 3B illustrates that ADC-6 and the SI-1×6.4 antibody maintain affinity for both the antigens EGFR and HER3-Fc.

FIG. 3C illustrates that ADC-219 and the SI-1×22 antibody maintain affinity for both the antigens EGFR and HER3-Fc.

FIG. 3D illustrates that ADC-227 and the SI-1×24 antibody maintain affinity for both the antigens EGFR and HER3-Fc.

FIG. 3E illustrates that ADC-235 and the SI-1×25 antibody maintain affinity for both the antigens EGFR and HER3-Fc.

FIG. 3F illustrates that ADC-243 and the SI-1×26 antibody maintain affinity for both the antigens EGFR and HER3-Fc.

FIG. 4A illustrates the in vitro efficacy of six naked antibodies as well as six ADCs in A431.

FIG. 4B illustrates the in vitro efficacy of six naked antibodies as well as six ADCs in BXPC-3.

FIG. 4C illustrates the in vitro efficacy of six naked antibodies as well as six ADCs in FaDu.

FIG. 4D illustrates the in vitro efficacy of six naked antibodies as well as six ADCs in HARA-B.

FIG. 4E illustrates the in vitro efficacy of six naked antibodies as well as six ADCs in HCC827.

FIG. 4F illustrates the in vitro efficacy of the six naked antibodies as well as the six ADCs in SW620.

FIG. 5A illustrates the results of in vivo efficacy experiments of ADC-6 and SI-1×6.4 naked antibody in the A431 single-tumor model.

FIG. 5B illustrates the results of in vivo efficacy experiments of ADC-6, SI-1×6.4 naked antibody, Cetuximab (Cet) and ADC-214 in the SW620 single-tumor model.

FIG. 5C illustrates the results of in vivo efficacy experiments of ADC-6 and SI-1×6.4 naked antibody in A431+SW620 heterogeneous tumors.

FIG. 6A illustrates the results of in vivo efficacy experiments of ADC-6, ADC-219, ADC-235, ADC-227 and ADC-112 in the A431 single-tumor model.

FIG. 6B illustrates the results of in vivo efficacy experiments of ADC-6, ADC-219, ADC-235, ADC-227 and ADC-112 in the BXPC-3 single-tumor model.

FIG. 7A illustrates the in vitro efficacy of SI-1×6.4, Cetuximab, ADC-6, ADC-214 and d3 on the human poorly differentiated lung cancer squamous cell carcinoma cell line Oka-c-1.

FIG. 7B illustrates the in vitro efficacy of SI-1×6.4, Cetuximab, ADC-6, ADC-214 and d3 on human lung squamous cell carcinoma cells SK-MES-1. Specific embodiments

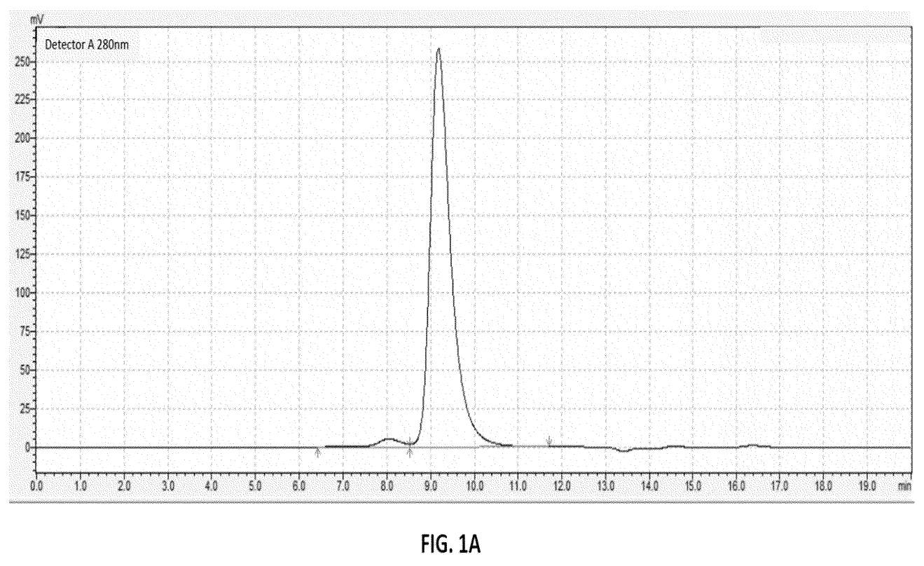

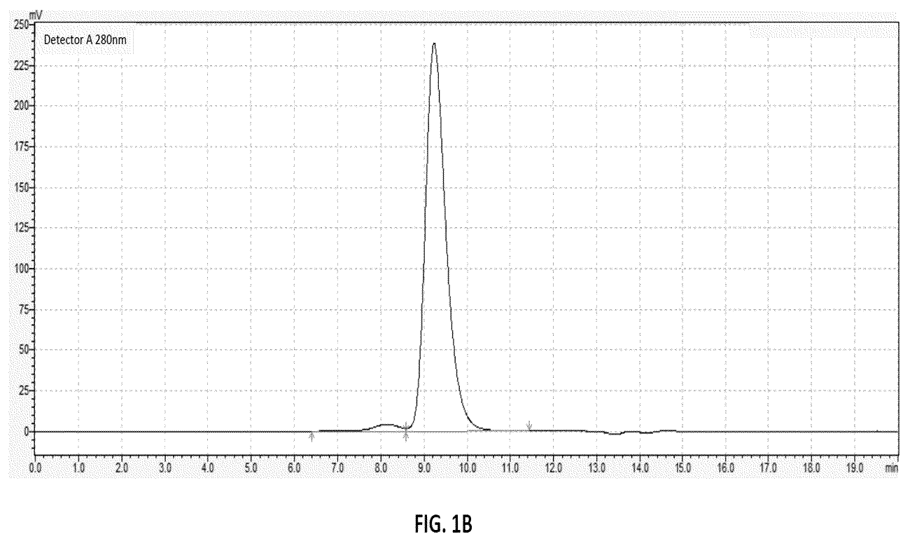

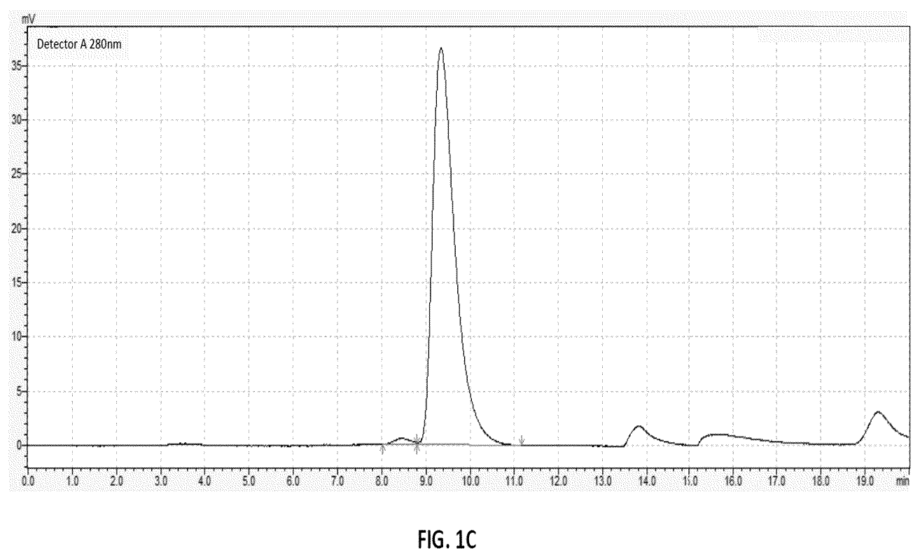

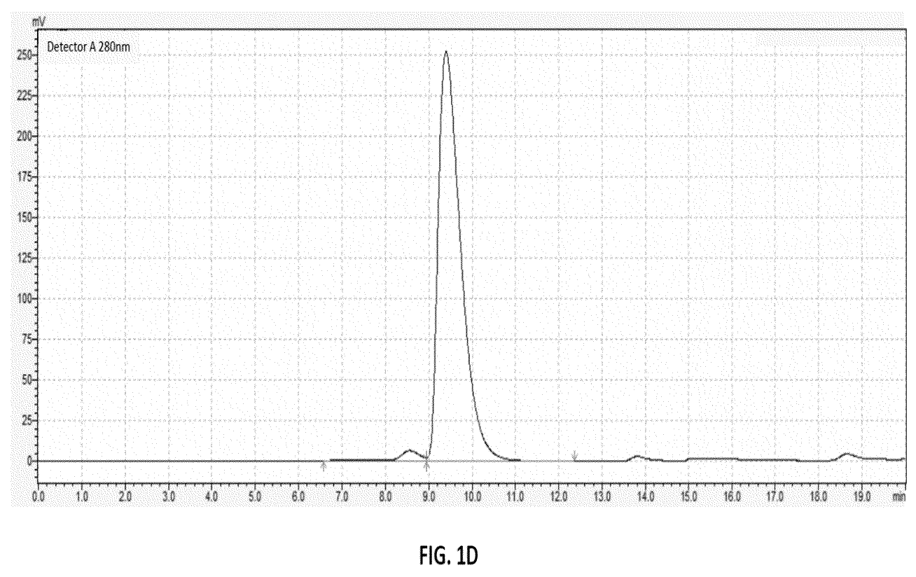

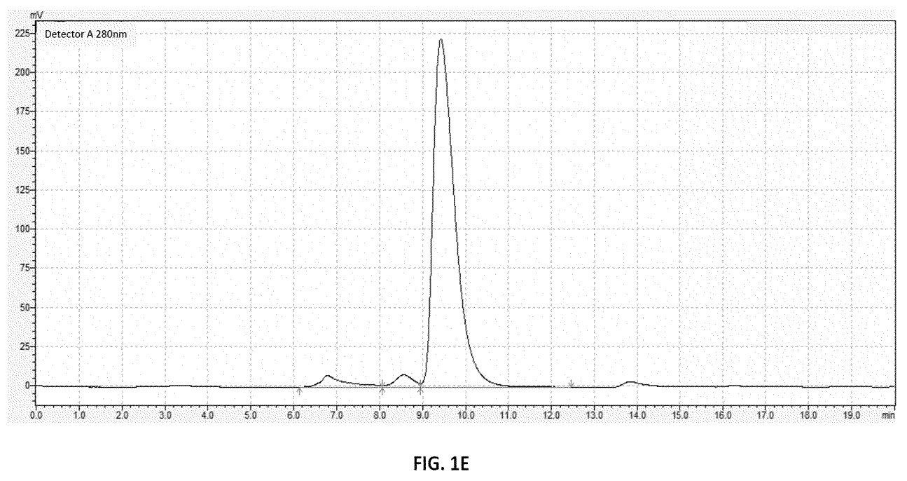

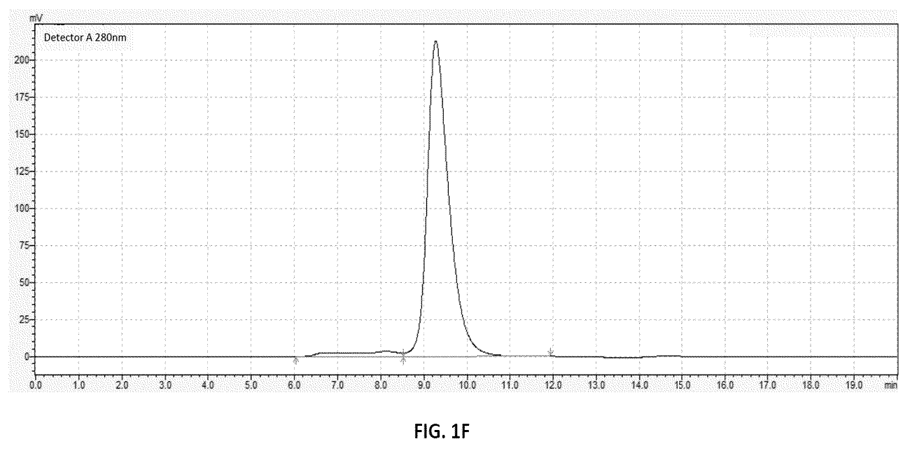

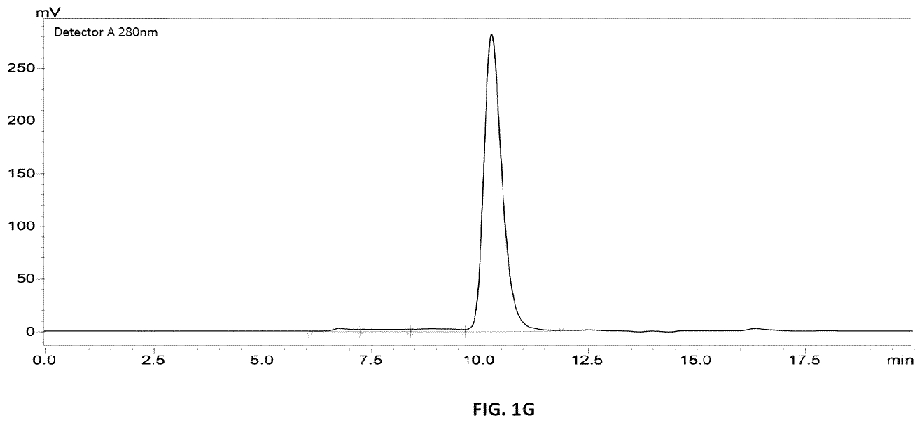

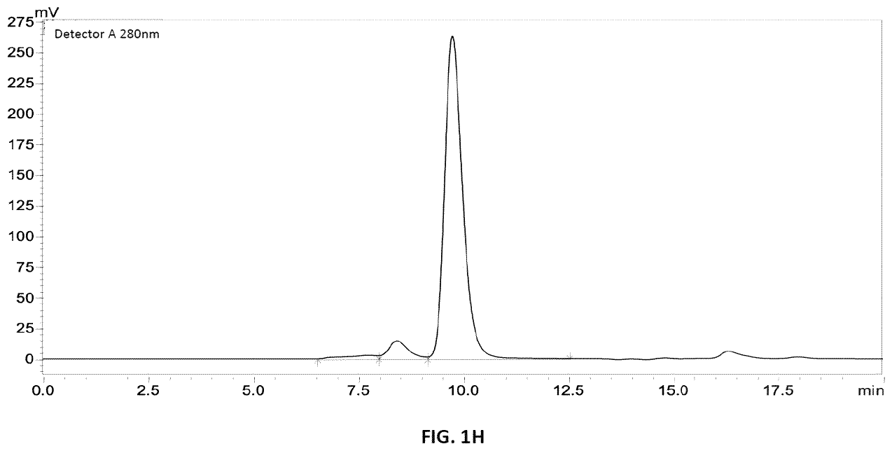

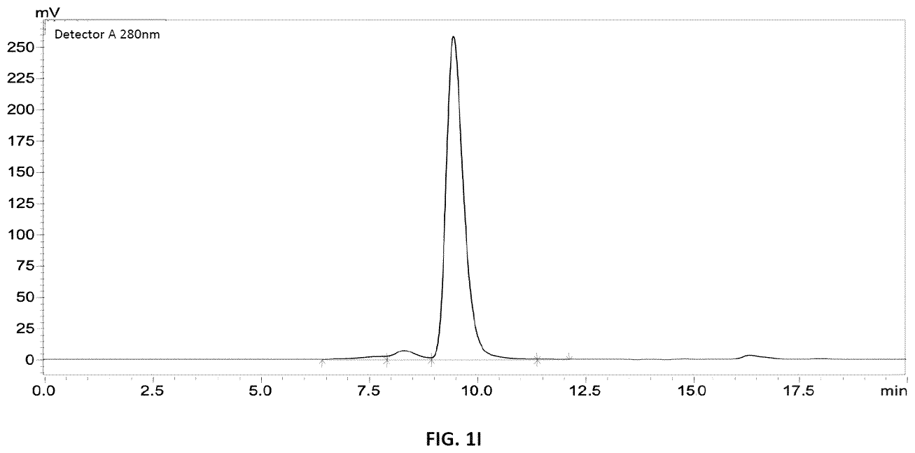

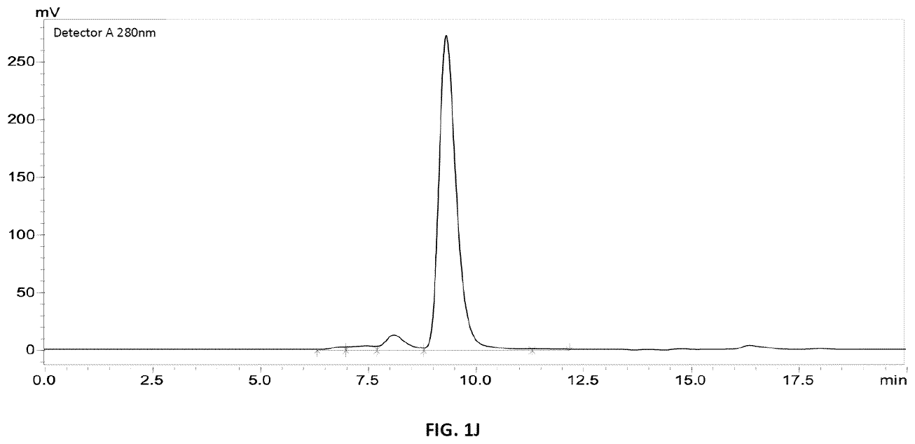

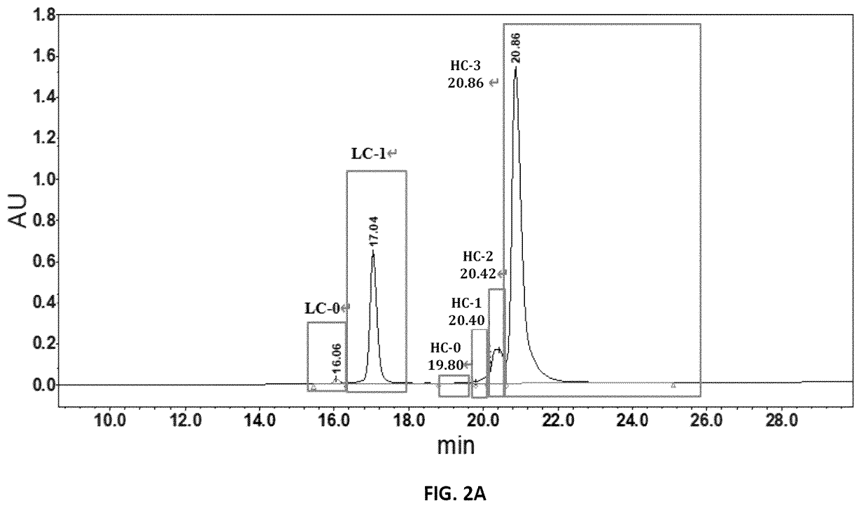

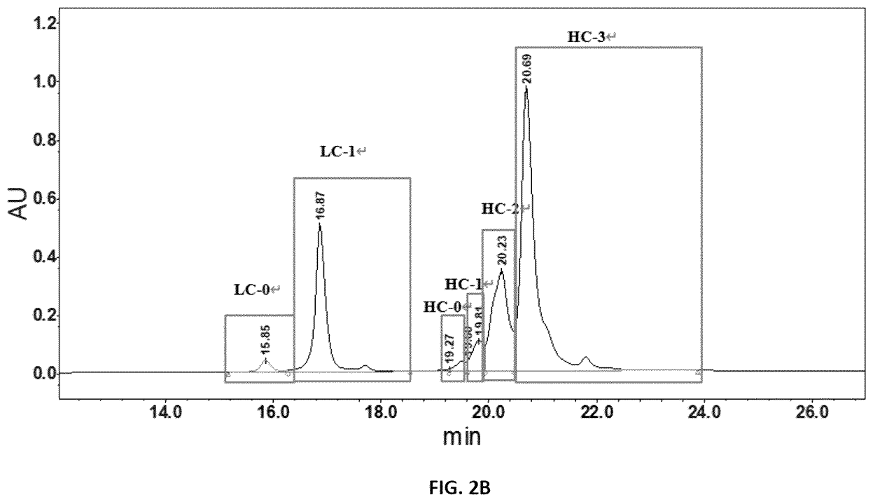

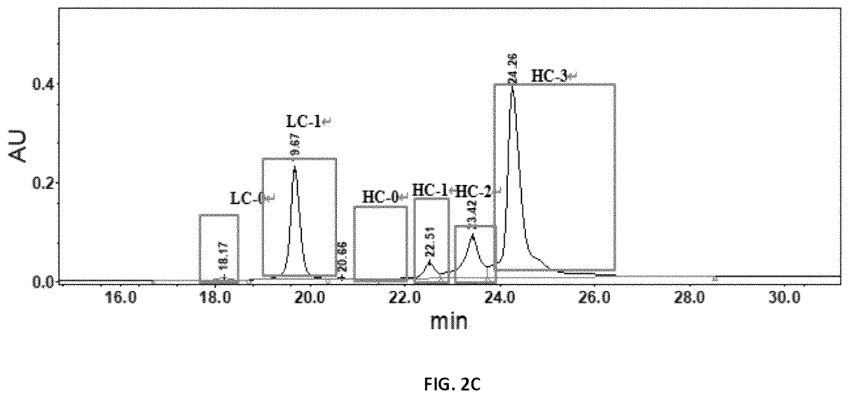

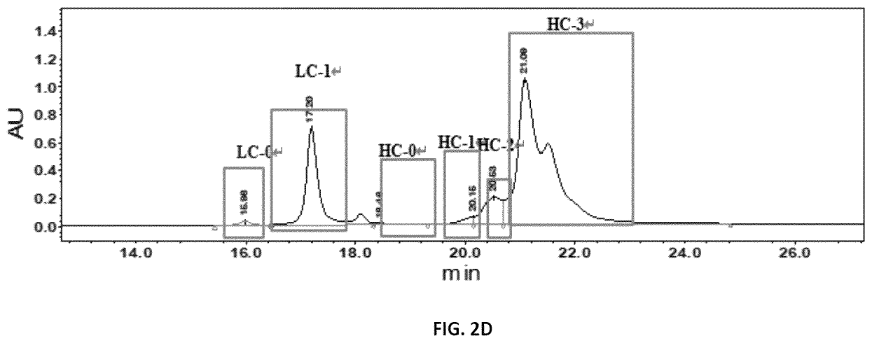

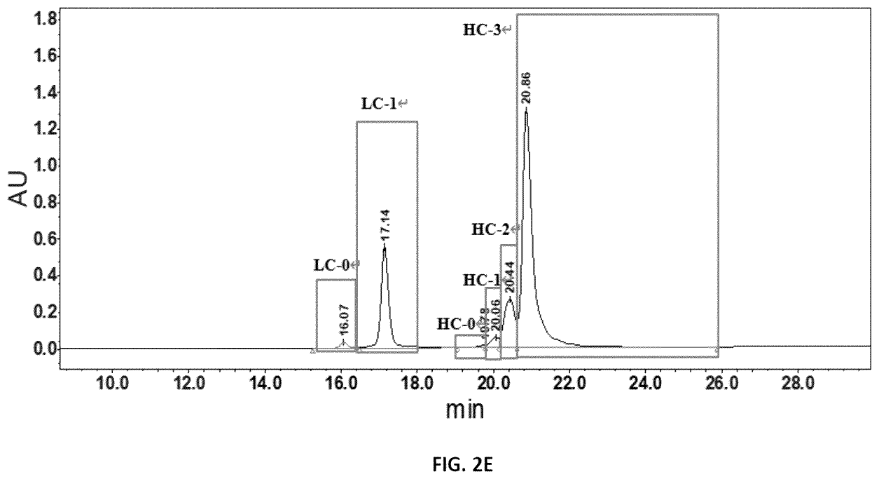

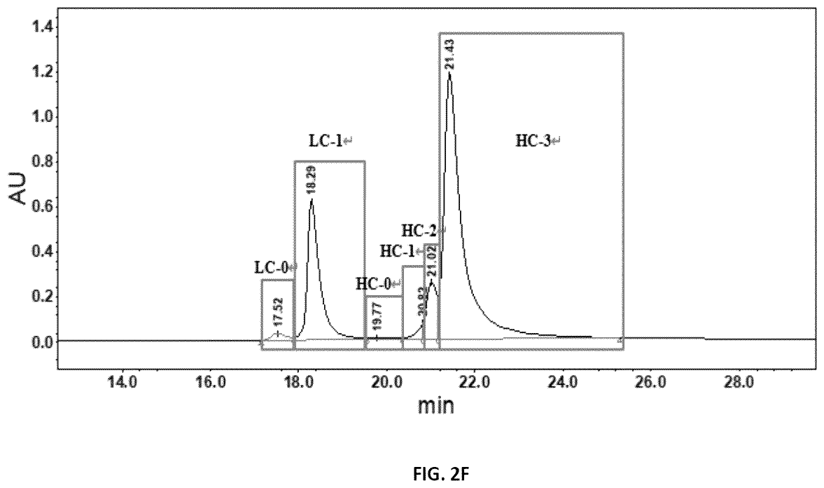

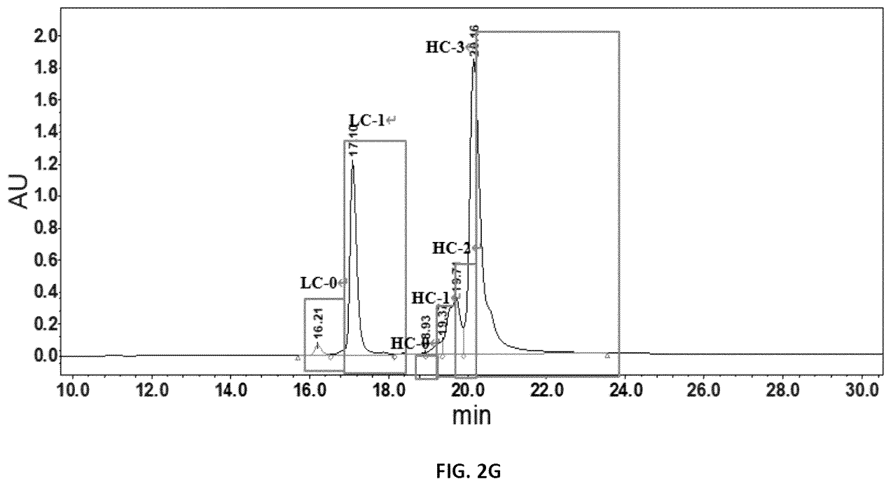

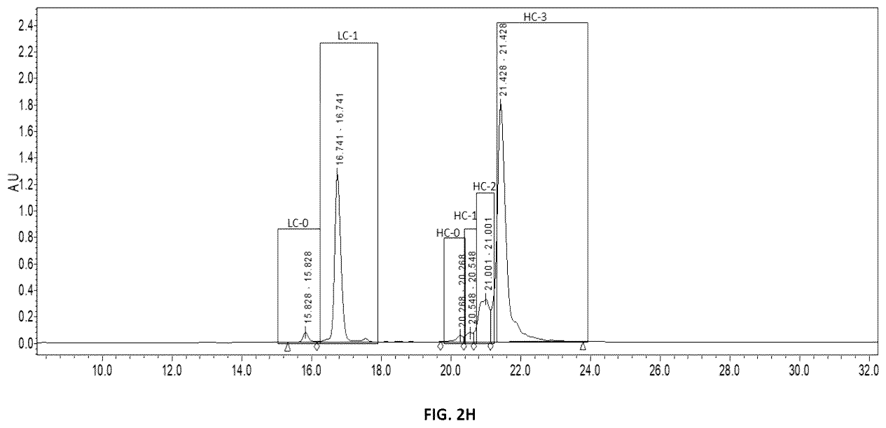

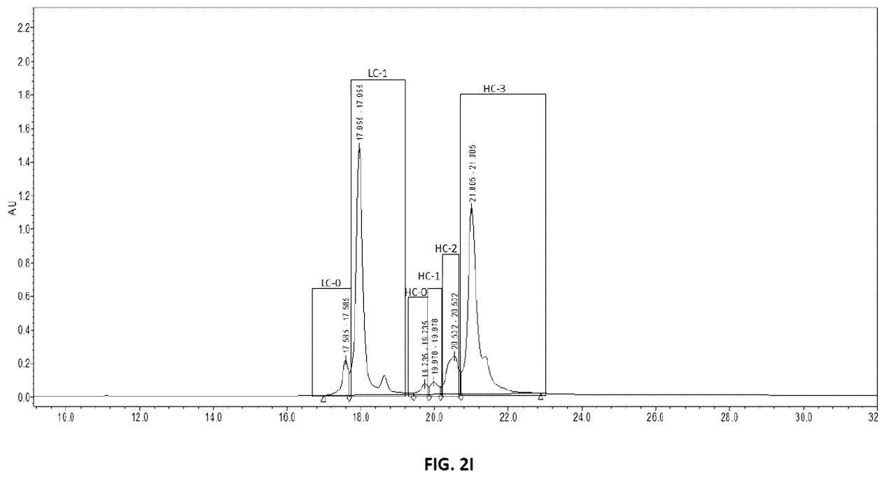

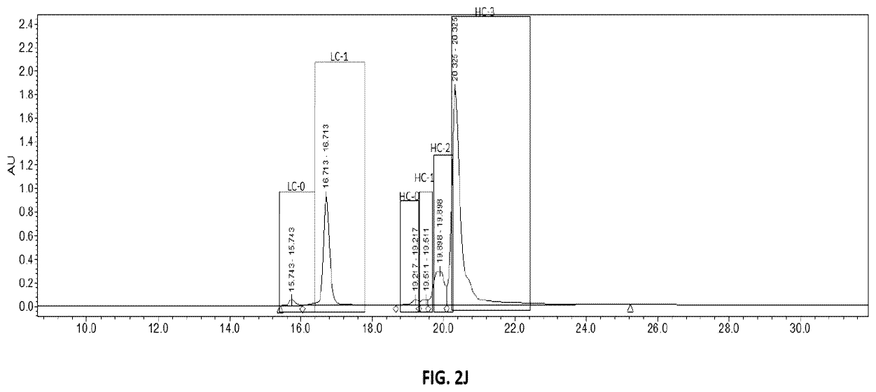

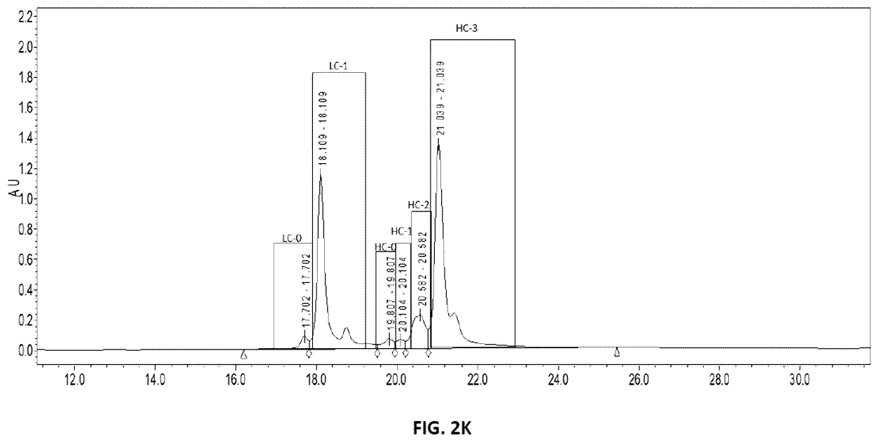

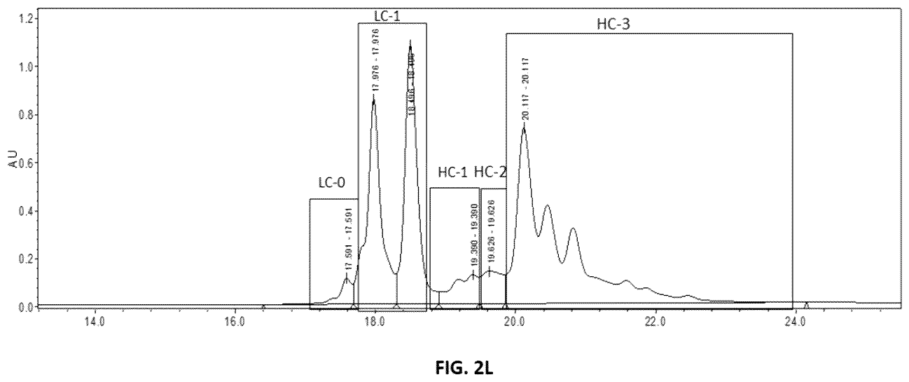

### ABBREVIATIONS AND DEFINITIONS

Unless otherwise indicated, the following terms and phrases, as used herein, are intended to have the meanings set forth below. When a trade name is used herein, unless otherwise indicated in the context, the trade name includes the product formula, generic drug and active ingredient of said trade name product.

Unless stated to the contrary, terms used in the claims and specification herein have the meanings set forth below.

The term “ligand” refers to a macromolecular compound that is able to recognize and bind to an antigen or receptor associated with a target cell. The ligand serves to present the drug to a target cell population bound to the ligand; these ligands include, but are not limited to, protein-like hormones, lectins, growth factors, antibodies, or other molecules capable of binding to cells. In embodiments of the present invention, the ligand is denoted as Ab, and the ligand may form a linkage bond with a linkage unit via a heteroatom on the ligand, and is preferably an antibody or an antigen-binding fragment thereof, said antibody being selected from chimeric, humanized, fully human, or murine antibodies; preferably monoclonal antibodies.

A ligand unit is a targeting agent that binds specifically to a target part. Said ligand is capable of specifically binding to a cellular component or binding to a cellular component or binding to other target molecules of interest. The target part or target is typically on the surface of the cell. In some aspects, the ligand unit serves to deliver the drug unit to a specific target cell population with which the ligand unit interacts. Ligands include, but are not limited to, proteins, polypeptides and peptides, and non-proteins such as sugars. Suitable ligand units include, for example, antibodies, such as full-length (intact) antibodies and antigen-binding fragments thereof. In embodiments where the ligand unit is a non-antibody targeting reagent, it may be a peptide or polypeptide, or a non-protein molecule. Examples of such targeting reagents include interferons, lymphokines, hormones, growth factors and colony stimulating factors, vitamins, nutrient transporter molecules, or any other cell-binding molecule or substance. In some embodiments, the linker is covalently attached to the sulfur atom of the ligand. In some aspects, the sulfur atom is a sulfur atom of a cysteine residue, and forms an interchain disulfide bond of the antibody. In another aspect, the sulfur atom is a sulfur atom of a cysteine residue that has been introduced into the ligand unit, and forms an interchain disulfide bond of the antibody. In another aspect, the sulfur atom is a sulfur atom of a cysteine residue that has been introduced into the ligand unit (e.g., by site-directed mutagenesis or chemical reaction). In other aspects, the linker-bound sulfur atom is selected from a cysteine residue that forms the interchain disulfide bond of the antibody or a cysteine residue that has been introduced into the ligand unit (e.g., by site-directed mutagenesis or chemical reaction). In some embodiments, the system is numbered according to the EU index in Kabat {[Kabat E. A et al, (1991)]“Sequences of proteins of Immunological Interest”, Fifth Edition, NIH Publication 91-3242}.

As used herein, “antibody” or “antibody unit” includes, to the extent thereof, any part of an antibody structure. This unit may bind, reactively associate, or complex with a receptor, antigen, or other receptor unit possessed by the target cell population. The antibody may be any protein or protein-like molecule, and can bind, complex, or react with a portion of the cell population to be treated or biologically modified. The antibodies forming the antibody-drug conjugates in the present invention maintain their original antigen-binding capacity from the wild state. Thus, the antibodies of the present invention are capable of binding exclusively to antigens. Antigens involved include, for example, tumor-associated antigens (TAA), cell surface receptor proteins and other cell surface molecules, cell survival regulators, cell proliferation regulators, molecules associated with tissue growth and differentiation (such as are known or foreseen to be functional), lymphokines, cytokines, molecules participating in the regulation of the cell cycle, molecules participating in angiogenesis, and molecules associated with angiogenesis (such as are known or foreseen to be functional). Tumor-associated factors may be cluster differentiation factors (e.g., CD proteins).

Antibodies applied in antibody drug conjugates include, but are not limited to, antibodies directed against cell surface receptors and tumor-associated antigens. Such tumor-associated antigens are well known in the industry and can be prepared by methods and information for antibody preparation well known in the industry. To develop effective cellular level targets that can be used in cancer diagnosis and therapy, researchers seek to find transmembrane or other tumor-associated peptides. These targets can be specifically expressed on the surface of one or more cancer cells with little or no expression on the surface of one or more non-cancer cells. Typically, such tumor-associated polypeptides are more overexpressed on the surface of cancer cells than on the surface of non-cancer cells. Confirmation of such tumor-associated factors can greatly enhance the specific targeting properties of antibody-based cancer therapies. For convenience, information related to antigens known to the industry is labeled below, including name, other names, and gene bank accession number. Nucleic acid and protein sequences corresponding to the tumor-associated antigens can be found in publicly available databases, such as Genbank. The tumor-associated antigens corresponding to antibody targeting include all amino acid sequence variants and isoforms having at least 70%, 80%, 85%, 90%, or 95% homology with the sequences confirmed in the references, or possessing biological properties and characteristics that are identical to those of the tumor-associated antigen sequences in the cited literature.

The term “inhibit” or “inhibition” means that a detectable amount is reduced or completely prevented.

The term “cancer” refers to a physiological condition or disease characterized by dysregulated cell growth. “Tumor” includes cancer cells.

The term “autoimmune disease” refers to diseases or disorders that originate in tissues or proteins that target an individual's own body.

The term “drug” refers to cytotoxic drugs, denoted by d, which are chemical molecules that have a strong ability to disrupt normal growth in tumor cells. Cytotoxic drugs can in principle kill tumor cells at sufficiently high concentrations, but due to a lack of specificity, they will also cause apoptosis of normal cells while killing tumor cells, leading to serious side effects. The term includes toxins, such as small molecule toxins or enzymatically active toxins of bacterial, fungal, plant or animal origin, radioisotopes (e.g., radioisotopes of At²¹¹, I¹³¹, I¹²⁵, Y⁹⁰, Re¹⁸⁶, Re¹⁸⁸, Sm¹⁵³, Bi²¹², P³² and Lu¹⁷⁶), toxic drugs, chemotherapeutic drugs, antibiotics and nucleolytic enzymes, preferably toxic drugs.

The term “linker” or “linker fragment” or “linker unit” refers to a fragment or bond of a chemical structure that is attached to a ligand at one end and to the drug at the other end, or may be attached to other connectors and then to the drug.

Connectors, including extensions, spacers, and amino acid units, can be synthesized by methods known in the art, such as those described in US2005-0238649A1. The connectors can be “cleavable connectors” that facilitate the release of the drug in the cell. For example, acid-unstable connectors (e.g., hydrazone), protease-sensitive (e.g., peptidase-sensitive) connectors, photo-unstable connectors, dimethyl connectors, or disulfide-containing connectors can be used (Chari et al. Cancer Research 52:127-131, 1992); U.S. Pat. No. 5,208,020.

According to the mechanism of intracellular drug release, as used herein, “linkers” or “linkers of antibody-drug conjugates” can be categorized into two types: unbreakable linkers and breakable linkers. For antibody-drug conjugates containing an unbreakable linker, the mechanism of drug release is as follows: after the conjugate binds to the antigen and is endocytosed by the cell, the antibody is cleaved enzymatically in lysosomes, releasing an active molecule consisting of the small-molecule drug, the linker, and amino acid residues of the antibody. The resulting change in the structure of the drug molecule does not diminish its cytotoxicity, but because the active molecule is electrically charged (amino acid residues), it cannot penetrate neighboring cells. Therefore, such active drugs cannot kill neighboring tumor cells that do not express the targeted antigen (antigen-negative cells) (bystander effect) (Ducry et al., 2010, Bioconjugate Chem. 21: 5-13). For antibody-drug conjugates containing a breakable linker, the mechanism of drug release is that after the conjugate binds to the antigen and is endocytosed by the cell, the conjugate breaks and releases the active ingredient (the small-molecule drug itself) in the target cell. Breakable linkers are mainly categorized into: chemical-sensitive linkers and enzyme-sensitive linkers. Chemically sensitive linkers can be selectively broken due to differences in the properties of the plasma and cytoplasm or tumor microenvironment. Such properties include pH, glutathione concentration, etc. pH-sensitive linkers, which are relatively stable in the neutral or weakly alkaline environment of blood (pH 7.3-7.5), will however be hydrolyzed within the weakly acidic tumor microenvironment (pH 5.0-6.5) and lysosomes (pH 4.5-5.0), e.g., hydrazones, carbonates, acetals, and ketals. Due to the limited plasma stability of acid-breakable linkers, antibody-drug conjugates based on such linkers typically have a short half-life (2-3 days). This short half-life has somewhat limited the use of pH-sensitive linkers in the new generation of antibody-drug conjugates. Glutathione-sensitive linkers are also known as disulfide-bond linkers. Drug release is based on the difference between the high intracellular glutathione concentration (millimolar range) and the relatively low glutathione concentration in the blood (micromolar range). This is particularly true for tumor cells, whose low oxygen content leads to enhanced reductase activity and thus to higher glutathione concentrations. Disulfide bonds are thermodynamically stable and therefore have better stability in plasma. Enzyme-unstable linkers, such as peptide linkers, provide better control of drug release. Peptide linkers can be effectively severed by lysosomal proteases such as cathepsins (Cathepsin B). This peptide linkage is thought to be very stable in the plasma circulation due to the unfavorable extracellular pH and serum protease inhibitors resulting in proteases that are normally inactive outside the cell. In view of the high plasma stability and good intracellular break selectivity and effectiveness, enzyme-unstable linkers are widely used as breakable linkers for antibody-drug conjugates.

The term “antibody-drug conjugate” refers to the attachment of an antibody to a biologically active drug by means of a stable linkage unit. In the context of the present invention, “ligand-drug conjugate”, preferably antibody-drug conjugate (ADC), refers to the attachment of a monoclonal antibody or antibody fragment to a biologically active toxic drug by means of a stable linkage unit.

The three-letter codes and single-letter codes for amino acids used in this disclosure are as described in J. boil. Chem. 1968, 243, 3558.

The term “alkyl” refers to a saturated aliphatic hydrocarbon group, which is a straight or branched chain group containing 1 to 20 carbon atoms (i.e., “C1-C20 alkyl”), preferably an alkyl group containing 1 to 12 carbon atoms (i.e., “C1-C12 alkyl”), more preferably an alkyl group containing 1 to 10 carbon atoms (i.e., “C1-C10 alkyl”), and most preferably an alkyl group containing 1 to 6 carbon atoms (i.e., “C1-C6 alkyl”). Non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and their various branched isomers, and the like. More preferred are lower alkyl groups containing 1 to 6 carbon atoms, and non-limiting embodiments include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, and the like. The alkyl group may be substituted or non-substituted, and when substituted, the substituent group may be substituted at any available point of attachment, said substituent group preferably being one or more of the following groups, independently selected from alkyl, alkenyl, alkynyl, alkoxy, alkylthio, alkylamino, halogen, mercapto, hydroxy, nitro, cyano, cycloalkyl, heterocycloalkyl, aryl, heteroaryl, cycloalkoxy, heterocycloalkoxy, cycloalkylthio, heterocycloalkylthio, oxo.

The term “substituted alkyl” means that a hydrogen in the alkyl group has been replaced by a substituent group. Unless otherwise indicated in the text, the substituent group of the alkyl group may be a variety of groups selected from the following group: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂, the number of substituents is from 0 to (2m′+1), where m′ is the total number of carbon atoms in the group. R′, R″ and R′″ each independently refer to hydrogen, unsubstituted C₁₋₈ alkyl, unsubstituted C6-C12 aryl (or C6-C10 aryl), C6-C12 aryl (or C6-C10 aryl) substituted by 1-3 halogens, unsubstituted C₁₋₈ alkyl, C₁₋₈ alkoxy or C₁₋₈ thioalkoxy, or unsubstituted C6-C12 aryl (or C6-C10 aryl)-C1-4 alkyl. When R′ and R″ are attached to the same nitrogen atom, they may form a 3-, 4-, 5-, 6-, or 7-membered ring together with that nitrogen atom. For example, —NR′R″ includes 1-pyrrolidinyl and 4-morpholinyl.

The term “alkylene” means a saturated straight or branched aliphatic hydrocarbon group, having two residues derived by removing two hydrogen atoms from the same carbon atom or two different carbon atoms of a parent alkane, and is a straight or branched group comprising 1 to 20 carbon atoms, preferably an alkylene group containing 1 to 12 carbon atoms, more preferably 1 to 6 carbon atoms. Non-limiting examples of alkylene groups include, but are not limited to, methylene (—CH₂—, 1,1-ethylidene (—CH(CH₃)—), 1,2-ethylidene (—CH₂CH₂)—, 1,1-propylidene (—CH(CH₂CH₃)—), 1,2-propylidene (—CH₂CH(CH₃)—), 1,3-propylidene (—CH₂CH₂CH₂—), 1,4-butylidene (—CH₂CH₂CH₂CH₂), and 1,5-butylidene (—CH₂CH₂CH₂CH₂CH₂—), among others. The alkylene group may be substituted or non-substituted, and when substituted, the substituent may be substituted at any available point of attachment, said substituent preferably being independently and optionally substituted with one or more substituents selected from alkyl, alkenyl, alkynyl, alkoxy, alkylthio, alkylamino, halo, mercapto, hydroxyl, nitro, cyano, cycloalkyl, heterocyclo, aryl, heteroaryl, cycloalkoxy, heterocycloalkoxy, cycloalkylthio, heterocycloalkylthio and oxo.

The term “alkoxy” refers to —O-(alkyl) and —O-(cycloalkyl), wherein alkyl or cycloalkyl is defined as above. Non-limiting examples of C1-C6 alkoxy include: methoxy, ethoxy, propoxy, butoxy, cyclopropoxy, cyclobutoxy, cyclopentyloxy, cyclohexyloxy. The alkoxy group may be optionally substituted or non-substituted, and when substituted, the substituent is preferably one or more of the following groups independently selected from alkyl, alkenyl, alkynyl, alkoxy, alkylthio, alkylamino, halogen, mercapto, hydroxyl, nitro, cyano, cycloalkyl, heterocycloalkyl, aryl, heteroaryl, cycloalkoxy, heterocycloalkoxy, cycloalkylthio, heterocycloalkylthio.

The term “cycloalkyl” refers to a saturated or partially unsaturated monocyclic or polycyclic cyclic hydrocarbon substituent, wherein the cycloalkyl ring comprises from 3 to 20 carbon atoms (i.e. “C3-C20 cycloalkyl”), preferably comprising from 3 to 12 carbon atoms (i.e. “C3-C12 cycloalkyl”), more preferably comprising 3 to 10 carbon atoms (i.e., “C3-C10 cycloalkyl”), most preferably comprising 3 to 8 carbon atoms (i.e., “C3-C8 cycloalkyl”). Non-limiting examples of monocyclic cycloalkyl groups (e.g., “C3-C8 cycloalkyl”) include cyclopropyl, cyclobutyl, cyclopentyl, cyclopentenyl, cyclohexyl, cyclohexenyl, cyclohexadienyl, cycloheptyl, cycloheptatrienyl, cyclooctyl, and the like; and polycyclic cycloalkyl groups include cycloalkyl groups of spirocycles, fused rings, and bridged rings.

The term “heterocyclyl” means a saturated or partially unsaturated monocyclic or polycyclic cyclic hydrocarbon substituent comprising from 3 to 20 ring atoms (i.e., a “3-20-membered heterocyclyl”), wherein one or more of the ring atoms is a heteroatom selected from nitrogen, oxygen or S(O)ₘ (wherein m is an integer from 0 to 2), but excluding ring portions of —O—O—, —O—S— or —S—S—), with the remaining ring atom(s) being carbon. Preferably, it comprises 3 to 12 ring atoms (i.e., a “3-12 membered heterocyclic group”), 1 to 4 of which are heteroatoms; more preferably, the cycloalkyl ring comprises 3 to 10 ring atoms (i.e., a “3-10 membered heterocyclic group”). Non-limiting examples of monocyclic heterocyclic groups (e.g., 3-7-membered heterocyclic groups) include pyrrolidinyl, piperidinyl, piperazinyl, morpholinyl, thiomorpholinyl, homopiperazinyl, and the like. Polycyclic heterocyclic groups include spiro, fused and bridged heterocyclic groups.

The term “cycloalkylalkyl” means that the alkyl group is substituted with one or more cycloalkyl groups, preferably with one cycloalkyl group, wherein alkyl is defined as above and wherein cycloalkyl is defined as above, for example, C3-C8 cycloalkyl C1-C6 alkyl.

The term “haloalkyl” means an alkyl group substituted with one or more halogens, wherein the alkyl group is as defined above, for example, halo C1-C6 alkyl.

The term “deuteroalkyl” means an alkyl group substituted with one or more deuterium atoms, wherein the alkyl group is as defined above, for example, deutero C1-C6 alkyl.

The term “C6-C12 aryl” refers to carbocyclic aromatic system groups having 6-12 carbon atoms.

The term “C6-C10 aryl” refers to carbocyclic aromatic system groups having 6-10 carbon atoms, such as phenyl, naphthyl, etc.

The term “5-10-membered heteroaryl” refers to aromatic heterocyclic rings, typically 5-, 6-, 7-, 8-, 9- and 10-membered heterocyclic rings having 1 to 3 heteroatoms selected from N, O, or S; the heteroaryl ring can optionally be further fused or attached to aromatic and non-aromatic carbon rings and heterocyclic rings. Non-limiting examples of said 5- to 10-membered heteroaryl rings are, for example, pyridinyl, pyrazinyl, pyrimidinyl, pyridazinyl, indolyl, imidazolyl, thiazolyl, isothiazolyl, thioxazolyl, pyrrolyl, phenyl-pyrrolyl, furanyl, phenyl-furanyl, oxazolyl, isoxazolyl, pyrazolyl, thiophenyl, benzofuranyl, benzothiophenyl, benzo 1,3-dioxolane (benzodioxole), isodihydroindolyl, benzimidazolyl, indazolyl, quinolinyl, isoquinolinyl, 1,2,3-triazolyl, 1-phenyl-1,2,3-triazolyl, 2,3-dihydroindolyl, 2,3-dihydrobenzofuranyl, 2,3-dihydrobenzothiophenyl, benzopyranyl, 2,3-dihydrobenzoxazinyl, 2,3-dihydroquinoxalinyl, and others.

The term “substituted C6-C10 aryl” or “substituted 5-10 membered heteroaryl” or “substituted 3-7 membered heterocyclyl” means that a hydrogen in the aryl or heteroaryl or heterocyclic group is replaced by a substituent group. Unless otherwise stated in the text, the substituent of the aryl or heteroaryl or heterocyclyl group may be a variety of groups selected from the following group: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂, with substituent numbers from 0 to (2m′+1), where m′ is the total number of carbon atoms in the group. R′, R″ and R′″ each independently refer to hydrogen, unsubstituted C1-8 alkyl, unsubstituted C6-C12 aryl (or C6-C10 aryl), C6-C12 aryl (or C6-C10 aryl) substituted by 1-3 halogens, unsubstituted C1-8 alkyl, C1-8 alkoxy or C1-8 thioalkoxy, or unsubstituted C6-C12 aryl (or C6-C10 aryl)-C1-4 alkyl. When R′ and R″ are attached to the same nitrogen atom, they may form a 3-, 4-, 5-, 6-, or 7-membered ring with that nitrogen atom. For example, —NR′R″ includes 1-pyrrolidinyl and 4-morpholinyl.

The term “hydroxyl” refers to the —OH group.

The term “halogen” means fluorine, chlorine, bromine, or iodine.

The term “amino” means —NH₂. The term “nitro” means —NO₂.

The term “amide group” means —C(O)N(alkyl) or (cycloalkyl), wherein alkyl and cycloalkyl are as defined above.

The term “carboxylate group” means —C(O)O(alkyl) or (cycloalkyl), wherein alkyl and cycloalkyl are as defined above.

The present invention also includes various deuterated forms of formula I. Each of the available hydrogen atoms attached to the carbon atoms may be independently replaced by a deuterium atom. A person skilled in the art can synthesize the deuterated form of formula I with reference to the relevant literature. Commercially available deuterated starting materials may be used in the preparation of deuterated forms of formula I, or they may be synthesized by conventional techniques using deuterated reagents, non-limiting examples of deuterated reagents including: deuteroborane, trideuteroborane tetrahydrofuran solution, deuterated lithium-aluminum hydride, deuterated ethyl iodide, and deuterated methyl iodide, and the like.

The term “antibody” refers to immunoglobulins, which are tetrapeptide chain structures consisting of two identical heavy chains and two identical light chains linked by interchain disulfide bonds. Immunoglobulins differ in the composition and order of amino acids in the constant region of the heavy chain, and therefore differ in their antigenicity. Accordingly, immunoglobulins can be categorized into five classes, or isoforms of immunoglobulins, i.e., IgM, IgD, IgG, IgA, and IgE, whose corresponding heavy chains are μ, δ, γ, α, and ε chains, respectively. The same class of Ig can be further divided into different subclasses according to differences in the amino acid composition of its hinge region and the number and positions of disulfide bonds of the heavy chain, e.g. IgG can be divided into IgG1, IgG2, IgG3 and IgG4. The light chains are divided into κ-chains or λ-chains by the differences in the constant region. Each of the five classes of Ig may have a κ chain or a λ chain. The antibodies described in the present invention are preferably specific antibodies against cell surface antigens on target cells, and non-limiting embodiments are the following antibodies: one or more of anti-EGFRvIII antibody, anti-DLL-3 antibody, anti-PSMA antibody, anti-CD70 antibody, anti-MUC16 antibody, anti-ENPP3 antibody, anti-TDGF1 antibody, anti-ETBR antibody, anti-MSLN antibody, anti-TIM-1 antibody, anti-LRRC15 antibody, anti-LIV-1 antibody, anti-CanAg/AFP antibody, anti-cladin 18.2 antibody, anti-Mesothelin antibody, anti-HER2 (ErbB2) antibody, anti-EGFR antibody, anti-c-MET antibody, anti-SLITRK6 antibody, anti-KIT/CD117 antibody, anti-STEAPI antibody, anti-SLAMF7/CS1 antibody, anti-NaPi2B/SLC34A2 antibody, anti-GPNMB antibody, anti-HER3(ErbB3) antibody, anti-MUC1/CD227 antibody, anti-AXL antibody, anti-CD166 antibody, anti-B7-H3(CD276) antibody, anti-PTK7/CCK4 antibody, anti-PRLR antibody, anti-EFNA4 antibody, anti-5T4 antibody, anti-NOTCH3 antibody, anti-Nectin 4 antibody, anti-TROP-2 antibody, anti-CD142 antibody, anti-CA6 antibody, anti-GPR20 antibody, anti-CD174 antibody, anti-CD71 antibody, anti-EphA2 antibody, anti-LYPD3 antibody, anti-FGFR2 antibody, anti-FGFR3 antibody, anti-FRα antibody, anti-CEACAMs antibody, anti-GCC antibody, anti-Integrin Av antibody, anti-CAIX antibody, anti-P-cadherin antibody, anti-GD3 antibody, anti-Cadherin 6 antibody, anti-LAMP1 antibody, anti-FLT3 antibody, anti-BCMA antibody, anti-CD79b antibody, anti-CD19 antibody, anti-CD33 antibody, anti-CD56 antibody, anti-CD74 antibody, anti-CD22 antibody, anti-CD30 antibody, anti-CD37 antibody, anti-CD138 antibody, anti-CD352 antibody, anti-CD25 antibody or anti-CD123 antibody.

The term “solvate” or “solvent compound” refers to the formation of a pharmaceutically usable solvate from the ligand-drug conjugate of the present invention with one or more solvent molecules, non-limiting examples of solvent molecules including water, ethanol, acetonitrile, isopropanol, DMSO and ethyl acetate.

The term “drug load” refers to the average number of cytotoxic drugs loaded on each antibody in formula I. It can also be expressed as the ratio of drug amount to antibody amount, and the drug load can range from 0-12, preferably 1-10 cytotoxic drugs (D) connected to each antibody (Ab).

In embodiments of the invention, the drug load is denoted as n, which exemplarily may be the mean value of 1, 2, 3, 4, 5, 6, 7, 8, 9, 10. The average number of drugs per ADC molecule after the coupling reaction can be identified by conventional methods such as UV/visible spectroscopy, mass spectrometry, ELISA tests and HPLC characteristics.

In one embodiment of the present invention, the cytotoxic drug is coupled to the open cysteine sulfhydryl-SH and/or the sulfhydryl-SH of the site-directed mutagenesis cysteine residue between the antibody chains by a linker unit, and generally the number of drug molecules that can be coupled to the antibody in the coupling reaction will be less than or equal to a theoretical maximum.

The loading of ligand cytotoxic drug conjugates can be controlled by the following non-limiting methods, including:
(1) controlling the molar ratio of the linking reagent to the monoclonal antibody;
(2) control of reaction time and temperature;
(3) selection of different reaction reagents.

For the preparation of conventional pharmaceutical compositions, see the Chinese Pharmacopoeia.

The term “pharmaceutically acceptable salt” or “pharmaceutically usable salt” means a salt of a ligand-drug conjugate of the present invention, or a salt of a compound described in the present invention; salts of this kind are safe and efficacious when used in a mammal, and are biologically active as desired. The ligand-drug conjugates of the present invention contain at least one carboxyl group, so they can form salts with bases, and non-limiting examples of pharmaceutically acceptable salts include sodium, potassium, calcium, or magnesium salts, etc.

The term “pharmaceutically acceptable salt” or “pharmaceutically usable salt” means a salt of an antibody-drug conjugate of the present invention, or a salt of a compound described herein; salts of this kind are safe and efficacious when used in a mammal, and are biologically active as desired.

The ligand-drug conjugates of the present invention contain at least one amino group and can therefore form salts with acids, non-limiting examples of pharmaceutically acceptable salts including: hydrochloride, hydrobromide, hydriodate, sulfate, bisulfate, citrate, acetate, succinate, ascorbate, oxalate, nitrate, phosphate, hydrogen phosphate, dihydrogen phosphate, salicylate, hydrogen citrate, tartrate, maleate, fumarate, formate, benzoate, methanesulfonate, ethanesulfonate, benzenesulfonate, p-toluenesulfonate.

“Acidic amino acid” refers to amino acids with an isoelectric point of less than 7. Acidic amino acid molecules tend to have one or more acidic groups, such as carboxyl groups, which can be effectively ionized into negative ionic forms in the structure to increase hydrophilicity. Acidic amino acids can be natural or unnatural.

The term “natural amino acids” refers to biologically synthesized amino acids. Natural amino acids are generally of the L-type, but there are a few exceptions, such as glycine, including natural and synthesized by organisms.

“Unnatural amino acids” means amino acids obtained by synthetic means.

The invention is further elaborated below in connection with specific embodiments which, it should be understood, are intended only to illustrate the invention, and are not intended to limit the scope of the invention. Test methods for which specific conditions are not indicated in the following embodiments are generally in accordance with conventional conditions or in accordance with conditions recommended by the manufacturer. All percentages, proportions, ratios or parts are by weight unless otherwise indicated.

### Example 1

### Synthesis of Compound M1

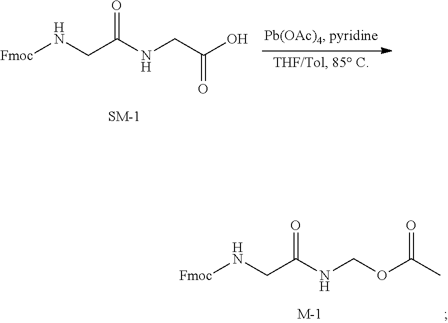

In a 5000 mL single-necked flask, N-fluorenylmethoxycarbonyl-glycine-glycine (100 g, 282 mmol, 1.0 eq), lead tetraacetate (175 g, 395 mmol, 1.4 eq), 2000 mL of dry tetrahydrofuran and 670 mL of toluene were added, stirred uniformly, protected by nitrogen, heated to 85° C. and reacted for 2.5 h. The reaction was monitored by TLC, and after the starting materials had finished reacting, cooling to room temperature and filtration were performed, the filtrate was concentrated under reduced pressure, and the residue was purified by column chromatography to obtain compound M1 (87 g); LC-MS: [M+NH₄]⁺=386.0.

### Example 2

### Synthesis of Compound M3

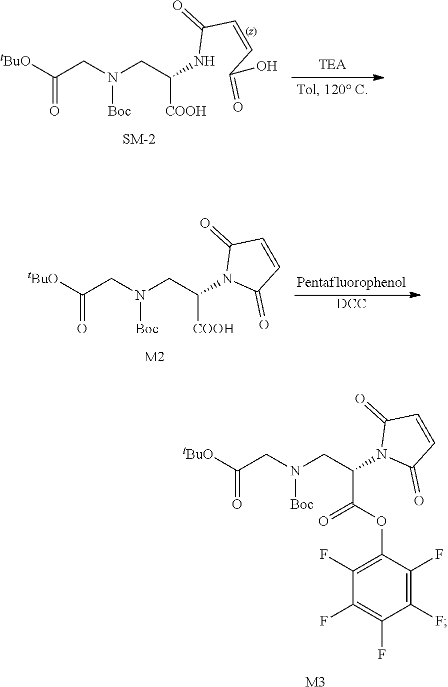

In a 1000 mL single-necked flask, compound SM-2 (synthesized according to the method published in patent CN108452321A) (40 g, 96 mmol, 1.0 eq), triethylamine (26.7 mL, 2.0 eq), and toluene (400 mL) were added, heated to 120° C., and a reaction was carried out by refluxing for 2 h. When TLC monitoring indicated that the reaction was essentially complete, cooling to 50° C. was performed, and solvent was removed by spinning under reduced pressure. Dissolution with ethyl acetate (150 mL) and water (40 mL) was performed, the pH was adjusted to 2-3 with 1M HCl under ice bath stirring, and liquid separation was performed. The aqueous layer was extracted once more with ethyl acetate, the organic layers were combined, and drying was performed by adding anhydrous sodium sulfate. After filtration, a light yellow oily crude product was obtained by concentration, and the crude product was purified by column chromatography (DCM:MeOH=40:1) to obtain compound M2 (26.6 g); LC-MS: [M+H]⁺=399.3.

In a 1000 mL single-necked flask, compound M2 (26.5 g, 60.5 mmol, 1.0 eq), pentafluorophenol (12.2 g, 66.5 mmol, 1.1 eq), DCC (13.7 g, 66.5 mmol, 1.1 eq), and THF (300 mL) were added, and a reaction was carried out at room temperature for 30 min (monitored by TLC). The insoluble material was filtered out. The reaction solution was purified directly by preparative LC, and the prepared solution was concentrated at 35° C. in a water bath under reduced pressure by pumping water to remove acetonitrile, and lyophilized to obtain compound M3 (31.5 g) with 64% yield; LC-MS: [M+H]⁺=565.1.

### Example 3

### Synthesis of the Compound Ent-M3

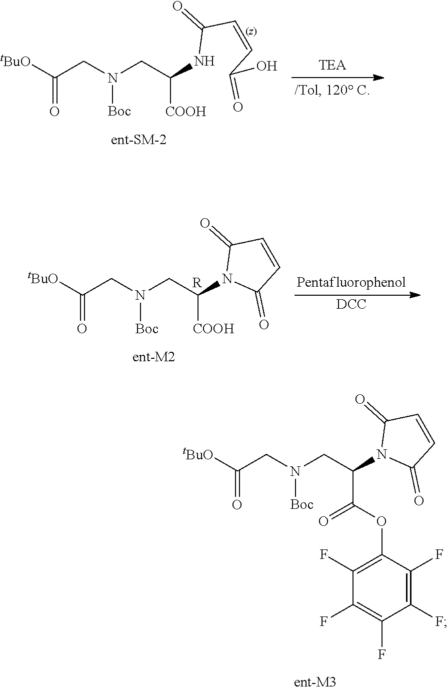

Referring to the synthetic route of Example 2, compound ent-M3 (27.8 g) was obtained; LC-MS: [M+H]⁺=565.2.

### Example 4

### Synthesis of Compound 1

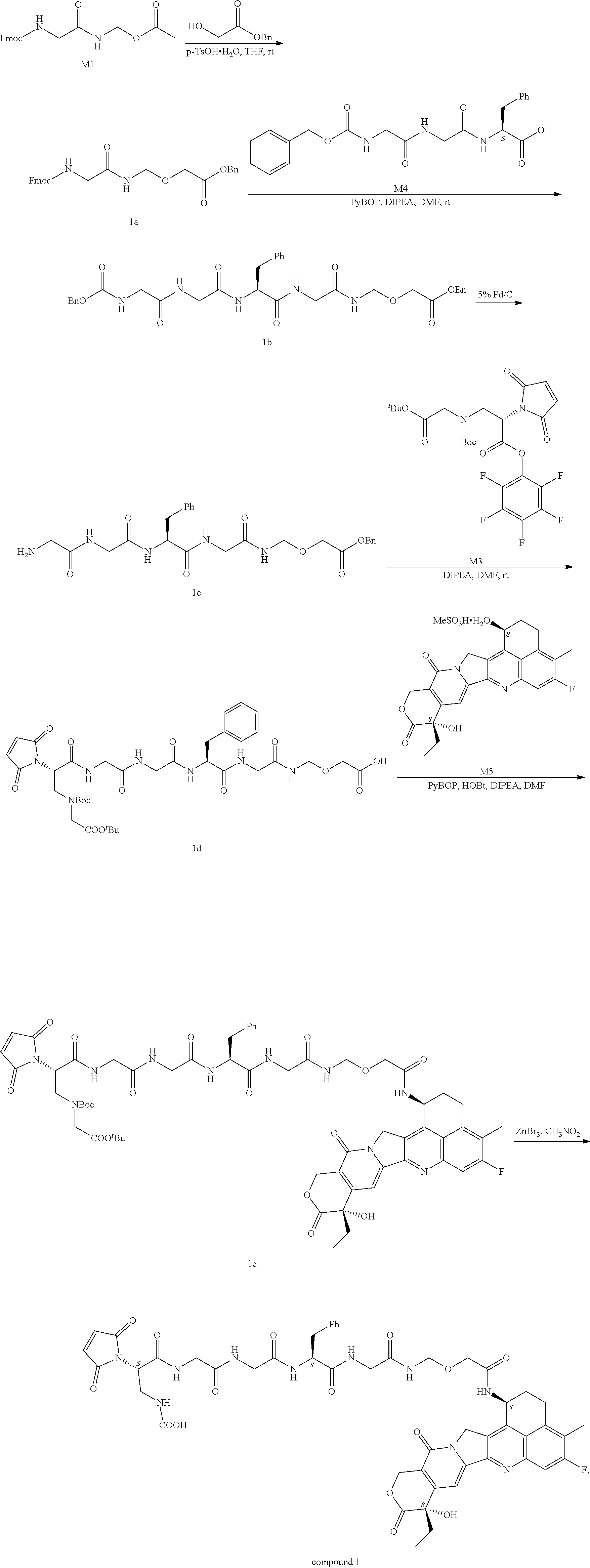

### Step 1: Compound 1a

In a 250 mL single-necked flask, add M1 (6 g, 16.3 mmol), 100 mL THF, p-toluenesulfonic acid monohydrate (0.31 g, 1.63 mmol), stir and cool to 0° C., perform dropwise addition of benzyl hydroxyacetate (5.4 g, 32.6 mmol), then naturally warm to room temperature for a reaction (the reaction lasts for about 2-4 h), with TLC monitoring. At the end of the reaction, saturated NaHCO₃ solution was added, extracted with ethyl acetate, washed with saturated sodium chloride solution, dried with anhydrous sodium sulfate, filtered, concentrated, and the residue was purified by silica gel column (PE:EA=10:1-5:1-1:1) to obtain 1a (4 g) at 52% yield; LC-MS: [M+H]⁺=475.18.

### Step 2: Compound 1b

In a 25 mL single-necked flask, add 1a (2 g, 4.2 mmol), 10 mL DMF, stir at 0° C., add DBU (766 mg, 5.04 mmol), and react for 1 h. After the completion of Fmoc deprotection as detected by TLC, the reaction solution was set aside ready for use.

M4 (prepared according to the method published in patent CN111051330 A) (1.73 g, 4.2 mmol), PyBOP (2.61 g, 5.04 mmol), HOBt (680 mg, 5.04 mmol) and 10 mL of DMF were added to another 25 mL single-necked flask, DIPEA (830 uL, 5.04 mmol) was added in an ice-water bath, stirring was continued for 30 min, the above reaction solution was added to the reaction flask, and the temperature was raised to room temperature for a reaction. When HPLC monitoring indicated the end of the reaction, the reaction solution was purified by preparative LC to obtain a product preparation liquid, the preparation liquid was extracted by dichloromethane, washed with saturated sodium chloride solution, dried with anhydrous sodium sulfate, filtered, and the filtrate was concentrated under reduced pressure to obtain solid 1b (1.7 g) at 63% yield; LCMS: [M+H]⁺=648.26.

### Step 3: Compound 1c

1b (900 mg, 1.39 mmol) was added to a 25 mL single-necked flask, and after dissolution with 15 mL of DMF, 900 mg of 5% Pd/C was added, and a hydrogenation reaction took place for 2 h. After the completion of the reaction, filtering was performed to obtain the filtrate, which was used directly for the next step of the reaction without purification.

### Step 4: Compound 1d

The crude product 1c was placed in an ice-water bath, DIPEA (235 uL, 1.39 mmol) was added, and compound M3 (784 mg, 1.39 mmol) was added, then the temperature was raised to room temperature to react for 1 h. When the reaction was complete as indicated by HPLC monitoring, the reaction solution was purified by HPLC to obtain a preparation liquid, which was lyophilized to obtain 1d (504 mg); LC-MS: [M+H]⁺=804.4.

### Step 5: Compound 1e

Add 1d (500 mg, 0.62 mmol), M5 (310 mg, 0.62 mmol), PyBOP (448 mg, 0.86 mmol), HOBt (116 mg, 0.86 mmol) and 15 mL DMF into a 50 mL single-necked flask, add DIPEA (378 uL, 2.29 mmol) in an ice-water bath, raise to room temperature and react for 2 h. After completion of the reaction was indicated by monitoring by HPLC, the reaction solution was purified by HPLC to obtain a preparation liquid of compound 1e, which was lyophilized to obtain 1e (210 mg); LC-MS: [M+H]⁺=1221.6.

### Step 6: Compound 1

1e (100 mg, 0.081 mmol), zinc bromide (368 mg, 1.63 mmol) and 5 mL of nitromethane were added to a 25 mL single-necked flask and the reaction was carried out at 40° C. for 1 h. After the completion of the reaction was indicated by monitoring by HPLC, concentration under reduced pressure was performed to remove the solvent, and a crude product was obtained. The crude product was purified by HPLC to obtain a product preparation liquid, and the preparation liquid was lyophilized to obtain the solid compound 1 (60 mg); LC-MS: [M+H]⁺=1065.3.

### Example 5

### Synthesis of Compound 2

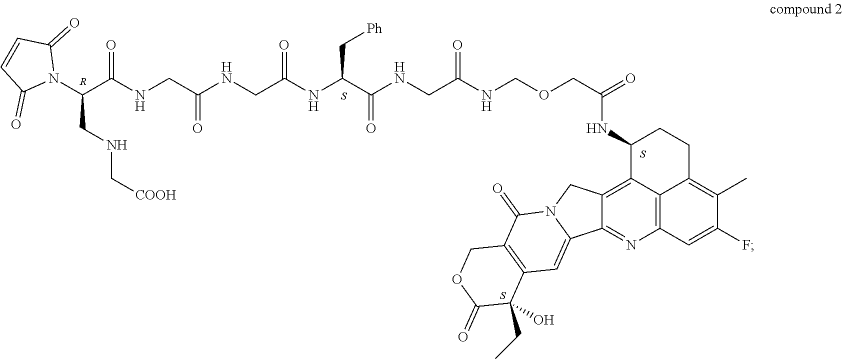

Referring to the synthetic route of Example 4, compound 2 (51 mg) was obtained; LC-MS: [M+H]⁺=1065.3.

### Example 6

### Synthesis of Compound 3

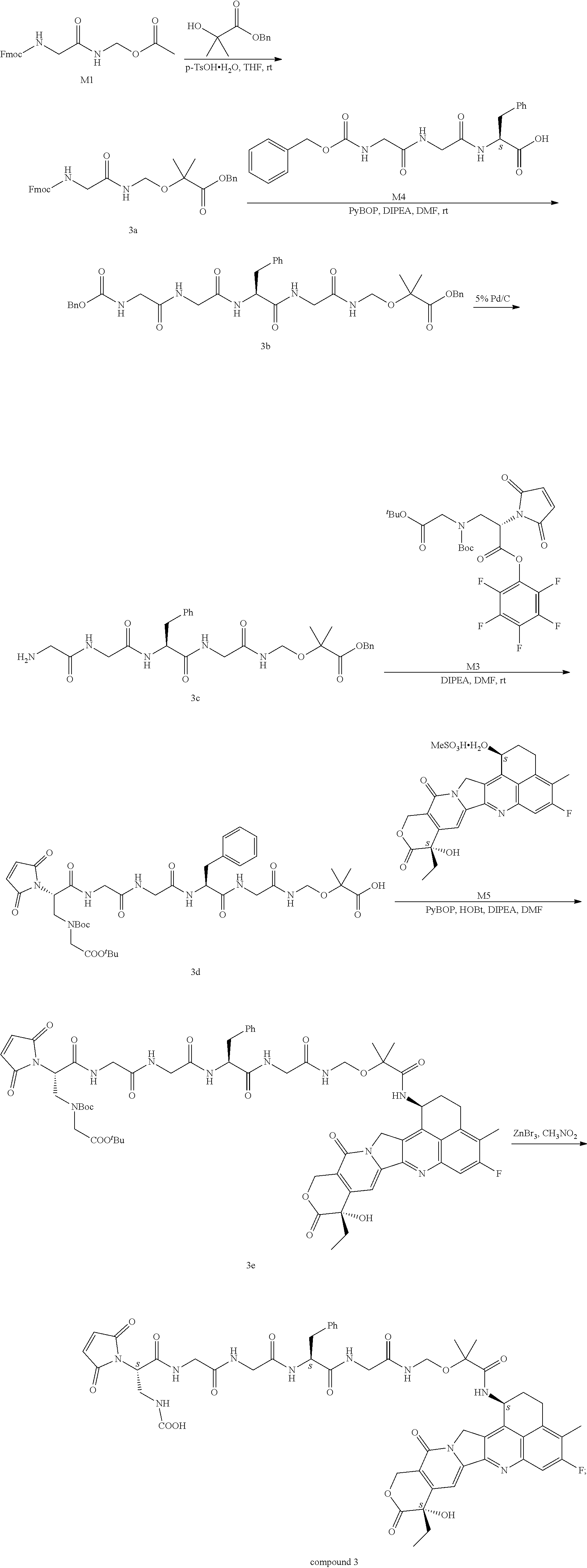

### Step 1: Compound 3a

In a 250 mL single-necked flask, add M1 (6 g, 16.3 mmol), 100 mL THF, p-toluenesulfonic acid monohydrate (0.31 g, 1.63 mmol), stir and cool to 0° C., perform dropwise addition of benzyl 2-hydroxy-2-methylpropionate (6.3 g, 32.6 mmol), then perform natural warming to room temperature for a reaction (the reaction lasts for about 2-4 h), with TLC monitoring. At the end of the reaction, saturated NaHCO₃ solution was added, followed by extraction with ethyl acetate, washing with saturated sodium chloride solution, drying with anhydrous sodium sulfate, filtering and concentration, and the residue was purified by silica gel column (PE:EA=10:1-5:1-2:1) to obtain 3a (4.2 g) at 52% yield; LC-MS: [M+H]⁺=503.3.

### Step 2: Compound 3b

In a 25 mL single-necked flask, add 3a (2 g, 4.0 mmol) and 10 mL DMF, stir at 0° C., add DBU (760 mg, 5.0 mmol), and react for 1 h. After the completion of Fmoc deprotection as detected by TLC, the reaction solution was set aside ready for use.

Add M4 (1.65 g, 4.0 mmol), PyBOP (2.59 g, 5.0 mmol), HOBt (675 mg, 5.0 mmol) and 10 mL of DMF into another 25 mL single-necked flask, and add DIPEA (823 uL, 5.04 mmol) in an ice-water bath, continue stirring for 30 min, and then add the above reaction solution to the reaction flask and raise to room temperature for a reaction. After the completion of the reaction as indicated by monitoring by HPLC, the reaction solution was purified by preparative LC to obtain a product preparation liquid, which was extracted by dichloromethane, washed with saturated sodium chloride solution, dried with anhydrous sodium sulfate and filtered, and the filtrate was concentrated under reduced pressure to obtain the solid 3b (1.4 g) at 53% yield; LC-MS: [M+H]⁺=676.2.

### Step 3: Compound 3c

Add 3b (700 mg, 1.04 mmol) to a 25 mL single-necked flask, dissolve in 10 mL of DMF, add 700 mg of 5% Pd/C, and perform a hydrogenation reaction for 1.5 h. After the completion of the reaction, filtering was performed to obtain a filtrate, which was used directly for the next step of the reaction without purification.

### Step 4: Compound 3d

Place the crude product 3c in an ice-water bath, add DIPEA (210 uL, 1.25 mmol) and then compound M3 (704 mg, 1.25 mmol), then raise to room temperature and react for 1 h. After the completion of the reaction as indicated by HPLC monitoring, the reaction solution was purified by HPLC to obtain a preparation liquid, which was lyophilized to give 3d (486 mg); LC-MS: [M−H]⁻=830.5.

### Step 5: Compound 3e

Add 3d (300 mg, 0.36 mmol), M5 (180 mg, 0.36 mmol), PyBOP (260 mg, 0.5 mmol), HOBt (67 mg, 0.5 mmol) and 10 mL of DMF to a 50 mL single-necked flask, add DIPEA (219.5 uL, 1.33 mmol) in an ice-water bath, raise to room temperature and react for 3 h. After the completion of the reaction as detected by HPLC, the reaction solution was purified by HPLC to give the preparation liquid of compound 3e, which was lyophilized to obtain 3e (157 mg); LC-MS: [M+H]⁺=1249.6.

### Step 6: Compound 3

Add 3e (100 mg, 0.08 mmol), zinc bromide (360 mg, 1.6 mmol) and 5 mL of nitromethane to a 25 mL single-necked flask, and react at 40° C. for 1 h. After the completion of the reaction as detected by HPLC, perform concentration under reduced pressure to remove the solvent, to obtain a crude product. The crude product was purified by high performance liquid chromatography to obtain a product preparation liquid, and the preparation liquid was lyophilized to obtain the solid compound 3 (64 mg); LC-MS: [M+H]⁺=1093.1.

### Example 7

### Synthesis of Compound 4

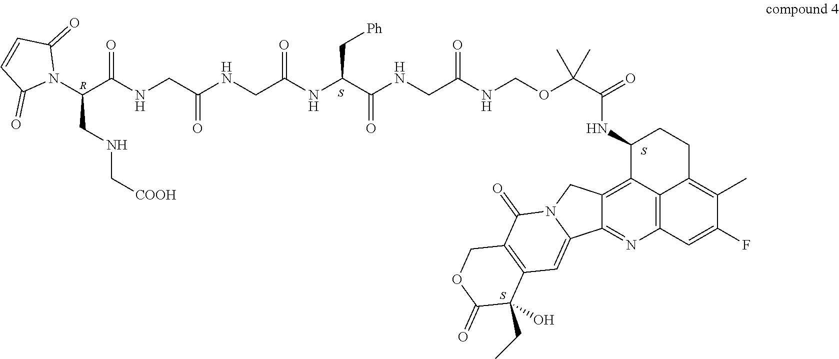

Referring to the synthetic route of Example 6, compound 4 (60 mg) was obtained; LC-MS: [M+H]⁺=1093.2.

### Example 8

### Synthesis of Compound 5A

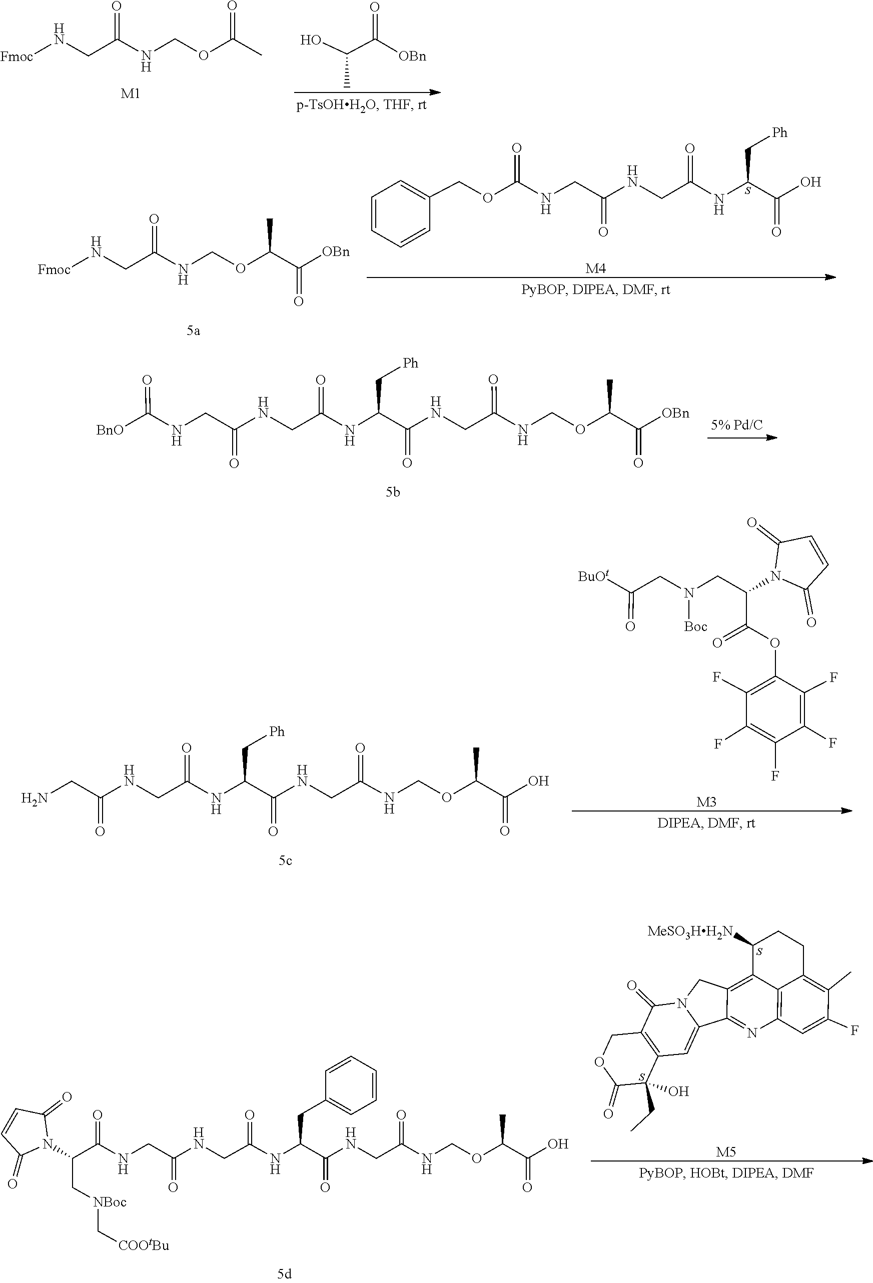

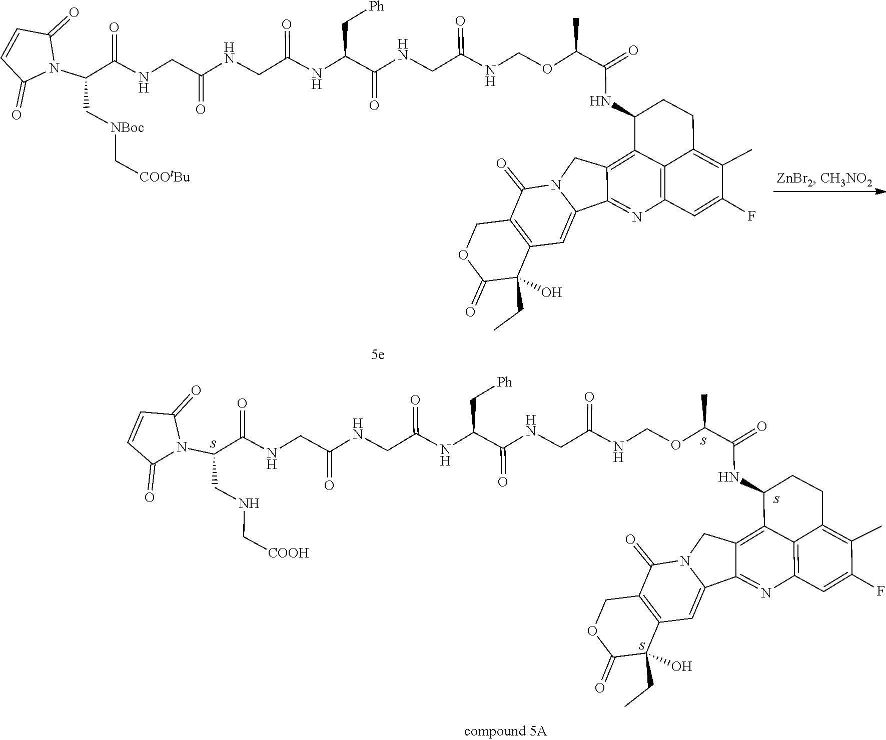

### Step 1: Compound 5a

Add M1 (500 mg, 1.4 mmol, 1.0 eq), p-toluenesulfonic acid monohydrate (26 mg, 0.1 mmol, 0.1 eq) and 10 mL of THF in a 25 mL single-necked flask, stir well, then lower to 0° C., and then slowly add L-lactic acid benzyl ester (1.2 g, 7.0 mmol, 5 eq), and then raise to room temperature for a reaction. TLC monitoring was performed, and at the end of the reaction, saturated NaHCO₃ solution was added, followed by extraction with ethyl acetate, drying over anhydrous sodium sulfate, filtering and concentration, and the residue was purified by reverse-phase column to give 5a (400 mg); LC-MS: [M+NH₄]⁺=506.2.

¹H NMR (400 Mz, CDCl₃/CD₃OD): 1.39 (3H, d, J=6.8 Hz), 3.78 (2H, t, J=4.0 Hz), 4.17-4.27 (2H, m), 4.42 (2H, d, J=4.0 Hz), 4.72-4.85 (2H, m), 5.11-5.58 (2H, m), 5.43 (1H, s), 7.06 (1H, t, J=8.0 Hz), 7.25-7.33 (6H, m), 7.38 (2H, t, J=8.0 Hz), 7.57 (2H, d, J=8.0 Hz), 7.75 (2H, d, J=8.0 Hz).

### Step 2: Compound 5b

Add Compound 5a (400 mg, 0.8 mmol, 1.0 eq) and 4 mL of DMF to a 25 mL single-necked flask, stir well, and then lower to 0° C. before slowly adding DBU (137 mg, 0.9 mmol, 1.1 eq). After the completion of the addition, raise to room temperature for a reaction. The reaction was monitored by TLC, and at the end of the reaction, the reaction solution was recorded as reaction solution \textcircled{1};

To another 25 mL single-necked flask, add M4 (372 mg, 0.9 mmol, 1.1 eq), PyBOP (852 mg, 1.6 mmol, 2.0 eq) and 3 mL of DMF, stir at room temperature for 5 min, and add reaction solution \textcircled{1}. The reaction was carried out at room temperature and monitored by HPLC. When the reaction was completed, the reaction solution was purified through HIPLC to yield compound 5b (326 mg); LC-MS: [M+NH₄]⁺=679.2.

### Step 3: Compound 5c

Add 5b (4.0 g, 6.05 mmol, 1.0 eq) to a 100 mL single-necked flask, dissolve in DMF (60 mL), then add 5% Pd/C (4 g), and perform a hydrogenation reaction at room temperature for 4 h (HPLC was used to monitor the progress of the reaction). The Pd/C was filtered, and the filtrate was placed directly in an ice-water bath (about 0° C.) without being concentrated, ready for use.

### Step 4: Compound 5d

Place the crude product 5c in an ice-water bath, add DIPEA (1.1 mL, 1.1 eq) and then compound M3 (3.4 g, 6.05 mmol), and then raise to room temperature and react for 2 h. After the completion of the reaction as detected by HIPLC, the reaction solution was purified by HPLC to obtain a preparative liquid, which was lyophilized to obtain 5d (3.15 g); LC-MS: [M−H]⁻=816.3.

### Step 5: Compound 5e

Add 5d (2.07 g, 2.53 mmol, 1.0 eq), M5 (1.35 g, 2.53 mmol, 1.0 eq), PyBOP (1.98 g, 3.79 mmol, 1.5 eq), HOBt (0.51 g, 3.79 mmol, 1.5 eq) and DMF (40 mL) to a 100 mL single-necked flask, add DIPEA (1.05 mL, 1.5 eq) in an ice-water bath, raise to room temperature and react for 2 h (monitored by HPLC). The reaction solution was directly purified by preparative LC, and the preparation liquid was concentrated in a water bath under reduced pressure by a water pump at 35° C. to remove acetonitrile and freeze-dried to obtain compound 5e (1.92 g), with a yield of 61%; LC-MS: [M+H]⁺=1235.4.

### Step 6: Compound 5A

To a 100 mL single-necked flask, add compound 5e (1.0 g, 0.8 mmol, 1.0 eq) and 35 mL nitromethane, and after dissolution, add zinc bromide (3.64 g, 16 mmol, 20.0 eq), react for 30 min in an oil bath at 40° C. (stabilized in advance by preheating), and concentrate at 45° C. in a water bath under reduced pressure by a water pump to remove the nitromethane, yielding a yellow residue solid (monitored by HPLC). After acid preparation, the preparation liquid of compound 5A was obtained, and was concentrated at 35° C. in a water bath under reduced pressure by a water pump to remove acetonitrile by spinning, and lyophilized to obtain compound 5A (786 mg) at a yield of 90%.

LC-MS: [M+H]⁺=1079.4;

¹H NMR (400 MHz, DMSO-d6) δ 9.39-9.02 (m, 1H), 8.70 (t, J=6.5 Hz, 1H), 8.64 (t, J=5.7 Hz, 1H), 8.56 (d, J=8.8 Hz, 1H), 8.34 (t, J=5.7 Hz, 1H), 8.16 (d, J=8.2 Hz, 1H), 8.01 (t, J=5.5 Hz, 1H), 7.71 (d, J=10.9 Hz, 1H), 7.30 (s, 1H), 7.28-7.15 (m, 4H), 7.14 (s, 2H), 5.53 (dd, J=14.5, 6.4 Hz, 1H), 5.49-5.34 (m, 2H), 5.22 (d, J=18.8 Hz, 1H), 5.09 (d, J=18.7 Hz, 1H), 5.03 (dd, J=9.6, 3.9 Hz, 1H), 4.73 (dd, J=9.9, 6.9 Hz, 1H), 4.59 (dd, J=10.1, 6.5 Hz, 1H), 4.49 (ddd, J=13.2, 8.6, 4.4 Hz, 1H), 4.14 (dd, J=13.3, 6.6 Hz, 2H), 3.93 (s, 2H), 3.84 (dd, J=16.5, 6.3 Hz, 1H), 3.76 (dd, J=16.9, 5.7 Hz, 2H), 3.70 (dd, J=5.2 Hz, 2H), 3.60 (dd, J=16.7, 5.4 Hz, 1H), 3.52 (dd, J=16.4, 5.1 Hz, 1H), 3.45 (dd, J=12.8, 10.1 Hz, 1H), 3.25-3.15 (m, 1H), 3.14-3.05 (m, 1H), 3.01 (dd, J=13.7, 4.1 Hz, 1H), 2.73 (dd, J=13.5, 9.8 Hz, 1H), 2.54-2.47 (m, 1H), 2.33 (s, 2H), 2.17 (d, J=5.5 Hz, 2H), 1.91-1.79 (m, 2H), 1.33 (d, J=6.6 Hz, 2H), 0.87 (t, J=7.3 Hz, 2H).

### Example 9

### Synthesis of Compound 5B

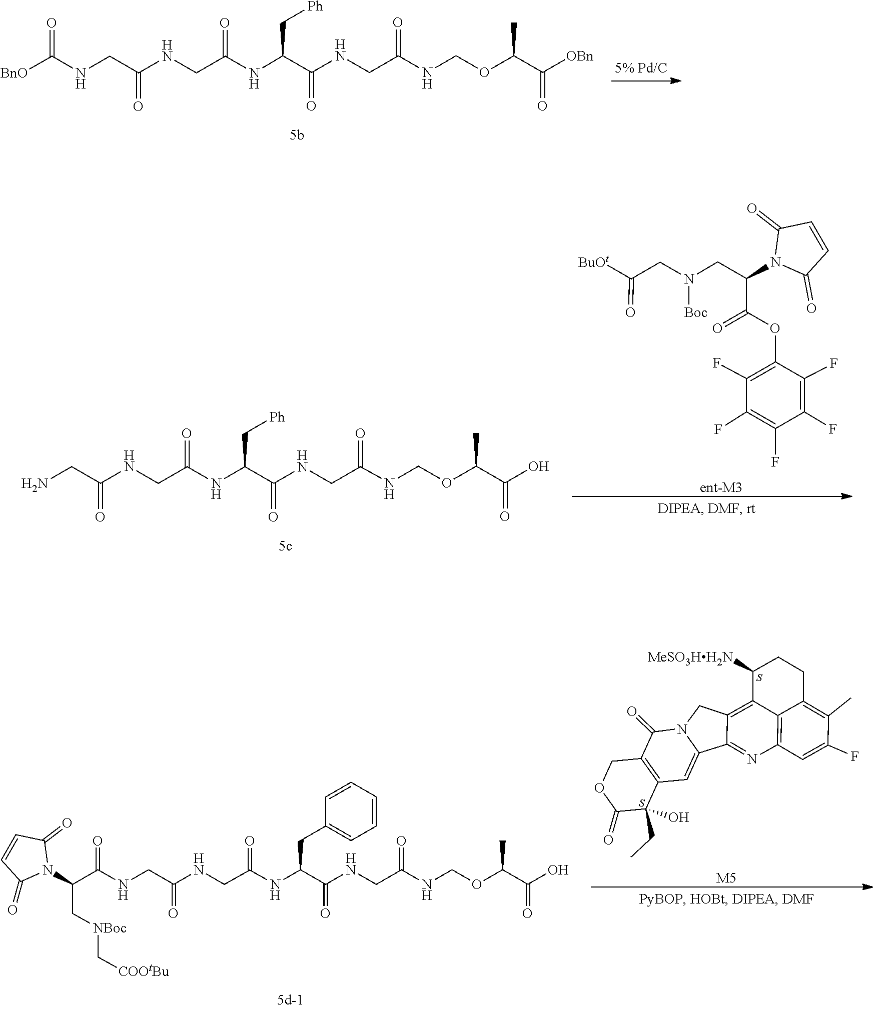

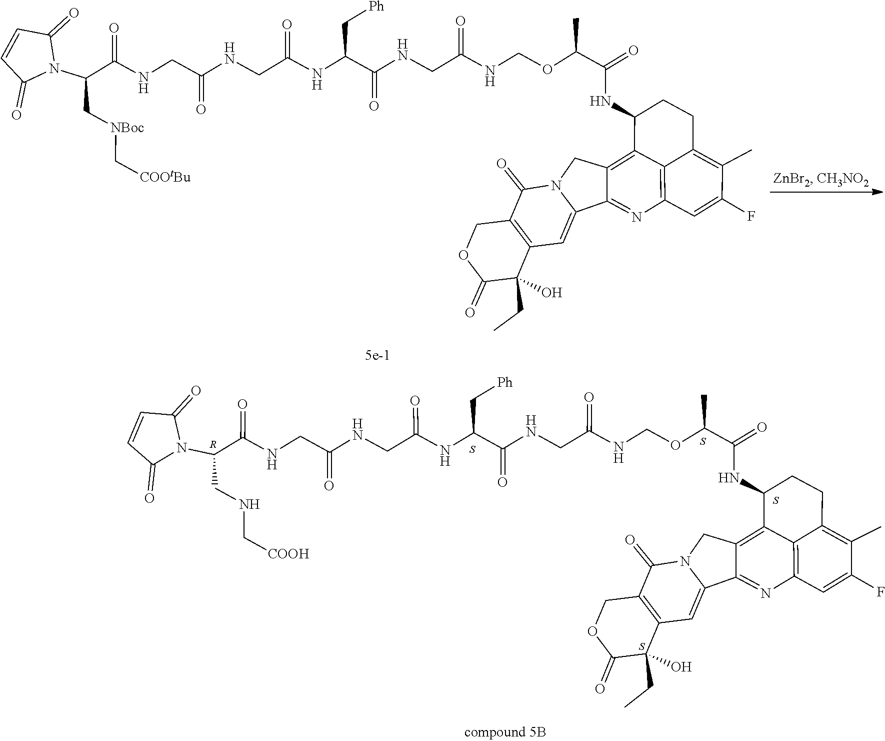

### Step 1: Compound 5d-1

Add compound 5b (300 mg, 0.45 mmol, 1.0 eq) and DMF (3 mL) to a 25 mL single-necked flask, stir to dissolve, add 5% Pd/C (300 mg), perform hydrogen replacement three times, perform a hydrogenation reaction for 2 h, and monitor for the end of the reaction by HIPLC. After the reaction is over, remove Pd/C by filtration, cool the filtrate to 0-5° C., add DIPEA (65 mg, 0.5 mmol, 1.1 eq), then add ent-M3 (255 mg, 0.45 mmol) to the filtrate, then raise to 20±5° C. and react for 1 h, and monitor for the end of the reaction by HIPLC. After the completion of the reaction, preparative purification was performed by HIPLC, and the product preparation liquid was collected and lyophilized to obtain compound 5d-1 (200 mg), with a yield of 54%; LC-MS: [M−H]⁻=816.3.

### Step 2: Compound 5e-1

Add compound 5d-1 (200 mg, 0.24 mmol, 1.0 eq), M5 (127 mg, 0.24 mmol, 1.0 eq), PyBOP (187 mg, 0.36 mmol, 1.2 eq), HOBt (48 mg, 0.36 mmol, 1.2 eq) and DMF (6 mL) into a 25 mL single-necked flask, lower the temperature in an ice-water bath to 0-5° C., and add DIPEA (62 mg, 0.48 mmol, 2.0 eq). After the addition is completed, raise to 20±5° C. and react for 2 h, and use HPLC to monitor for the completion of the reaction. The reaction solution underwent preparative purification by HPLC, and the product preparation liquid was collected and lyophilized to obtain compound 5e-1 (162.8 mg); LC-MS: [M+H]⁺=1235.4.

### Step 3: Compound 5B

Sequentially add compound 5e-1 (110 mg, 0.089 mmol, 1.0 eq), ZnBr₂ (400 mg, 1.78 mmol, 20.0 eq) and CH₃NO₂ (10 mL) into a 25 mL single-necked flask. After the addition, raise to 40° C. and react for 0.5 h, and then stop the reaction. The reaction solution was dried directly at 45° C. by spin-drying under reduced pressure to obtain a yellow solid. Samples were taken and the reaction was monitored by HPLC. The spin-dried solid was directly purified by HPLC preparation, and the product preparation liquid was collected and lyophilized to obtain compound 5B (73.4 mg) at 76.5% yield; LC-MS: [M+H]⁺=1079.4.

### Example 10

### Preparation of Compound 6A

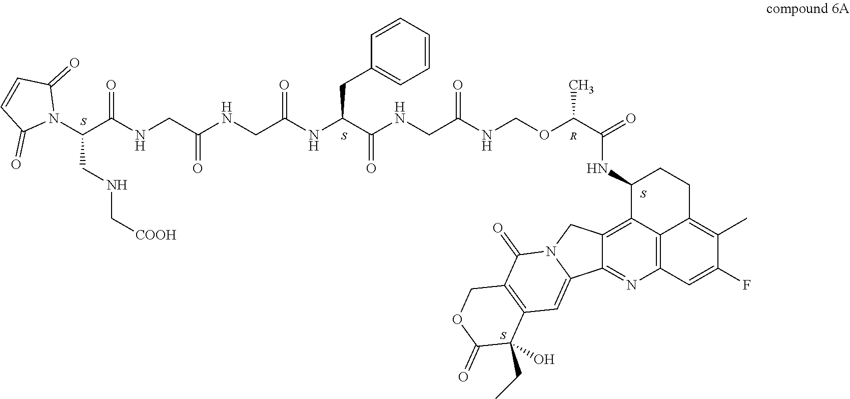

Referring to the synthetic route of Example 8, compound 6A (71 mg) was obtained; LC-MS: [M+H]⁺=1079.4.

### Example 11

### Preparation of Compound 6B

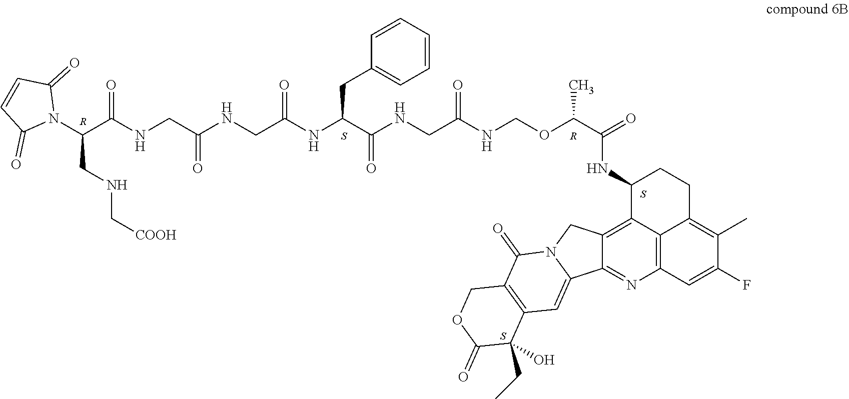

Referring to the synthetic route of Example 9, compound 6B (59 mg) was obtained; LC-MS: [M+H]⁺=1079.4.

### Example 12

### Preparation of Compounds 7A and 7B

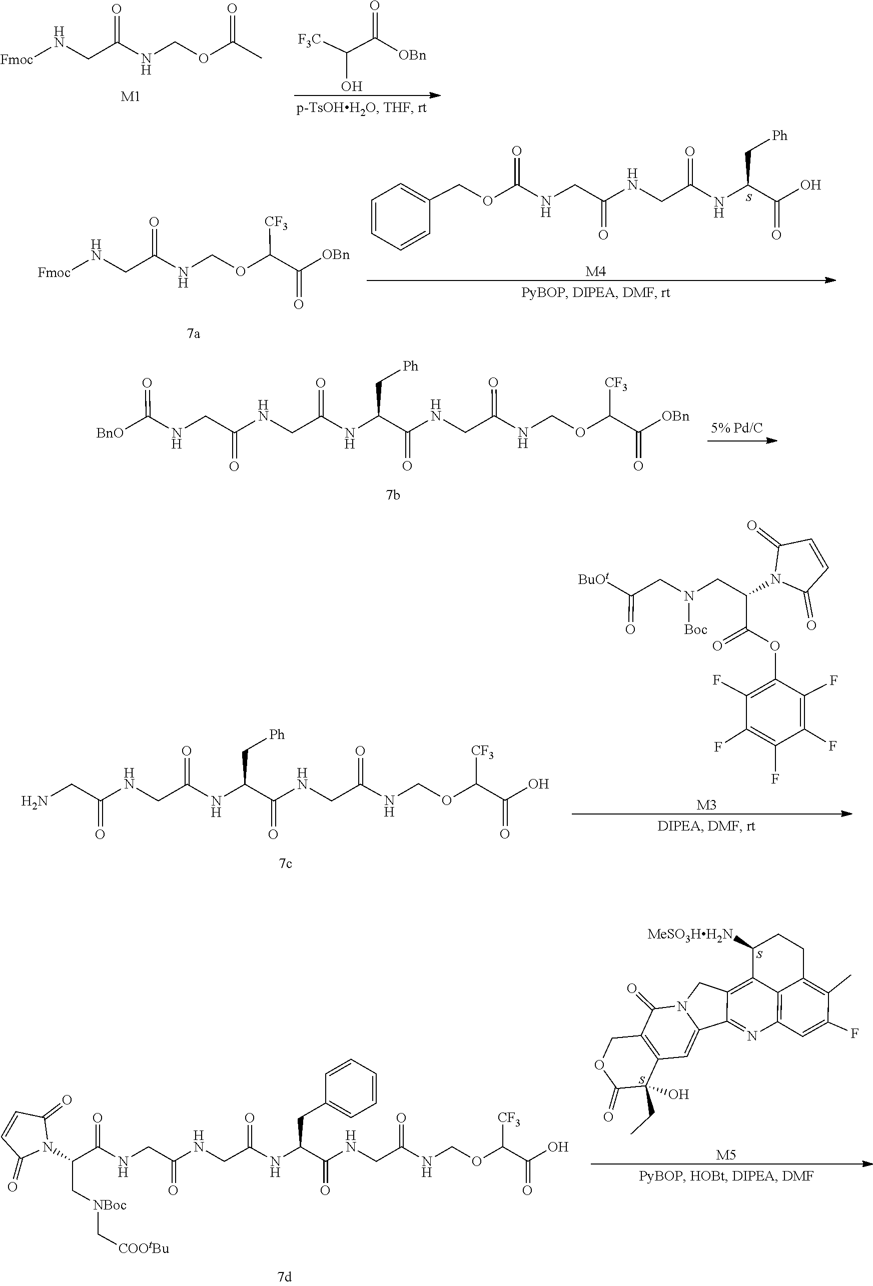

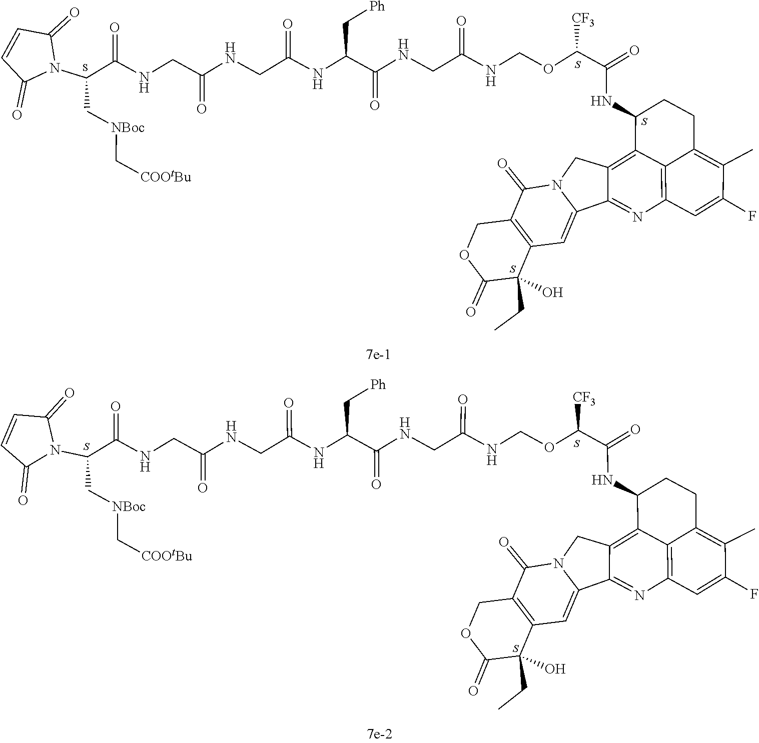

### Step 1: Compound 7a

In a 250 mL single-necked flask, add M1 (10 g, 27.1 mmol), benzyl 3,3,3-trifluorolactate (prepared according to the method published in patent WO2020063673A1) (12.7 g, 54.3 mmol), zinc acetate (9.96 g, 54.3 mmol) and 100 mL of toluene, heat to 100° C. and react for 4 h. When the reaction is completed, reduce to room temperature, filter to remove insoluble material, and concentrate the filtrate to obtain a crude product. The crude product was purified by silica gel column chromatography (PE:EA=10:1-5:1-2:1) to obtain 5.15 g of the target material, yield 35.1%; LC-MS: [M+H]⁺=543.17.

### Step 2: Compound 7b

Add 7a (5 g, 9.2 mmol) and 15 mL of DMF in a 50 mL single-necked flask, dissolve until clear, and then add DBU (1.68 g, 11 mmol) in an ice-water bath and react for 1 h, and record the reaction solution as reaction solution \textcircled{1}.

Take another 50 mL single-necked flask, add M4 (3.8 g, 9.2 mmol), PyBOP (5.75 g, 11 mmol), HOBt (1.49 g, 11 mmol) and 10 mL DMF. When dissolved, add DIPEA (1.82 mL, 11 mmol) in an ice-water bath, and continue the reaction for 30 min, then add reaction solution \textcircled{1}, and raise to room temperature and react for 2 h. The reaction progress was monitored by HPLC. After the completion of the reaction, the reaction solution was purified by high performance liquid chromatography to obtain a preparation liquid. The preparation liquid was extracted with dichloromethane, washed with saturated sodium chloride solution, dried with anhydrous sodium sulfate, filtered, and concentrated to obtain 4.1 g of solid, with a yield of 62.3%; LC-MS: [M+H]⁺=716.25.

### Step 3: Compound 7d

Add 7b (900 mg, 1.26 mmol) in a 25 mL single-necked flask, and after dissolution with 15 mL of DMF, add 900 mg of 5% Pd/C, and perform a hydrogenation reaction for 2 h. After the completion of the reaction, perform filtration, place the filtrate in an ice-water bath, add DIPEA (228 uL, 1.38 mmol), and then add M3 (712 mg, 1.26 mmol), and then raise to room temperature and react for 1 h. When the reaction was completed as detected by HIPLC, the reaction solution was purified by high performance liquid chromatography to obtain a preparation liquid, which was lyophilized to obtain 525 mg of a product at 47.9% yield; LC-MS: [M−H]⁻=870.33.

### Step 4: Compound 7e

Add 7d (500 mg, 0.57 mmol), M5 (305 mg, 0.57 mmol), PyBOP (448 mg, 0.86 mmol), HOBt (116 mg, 0.86 mmol) and 15 mL of DMF to a 50 mL single-necked flask, add DIPEA (378 uL, 2.29 mmol) in an ice-water bath, raise to room temperature and react for 2 h. After the completion of the reaction as detected by HIPLC, the reaction solution was purified by high performance liquid chromatography to obtain the preparation liquids of compound 7e-1 and compound 7e-2, and the preparation liquids were lyophilized to obtain 150 mg of compound 7e-1, LC-MS: [M+H]⁺=1289.46, and 220 mg of compound 7e-2, LC-MS: [M+H]⁺=1289.46, respectively.

### Step 5: Compound 7A

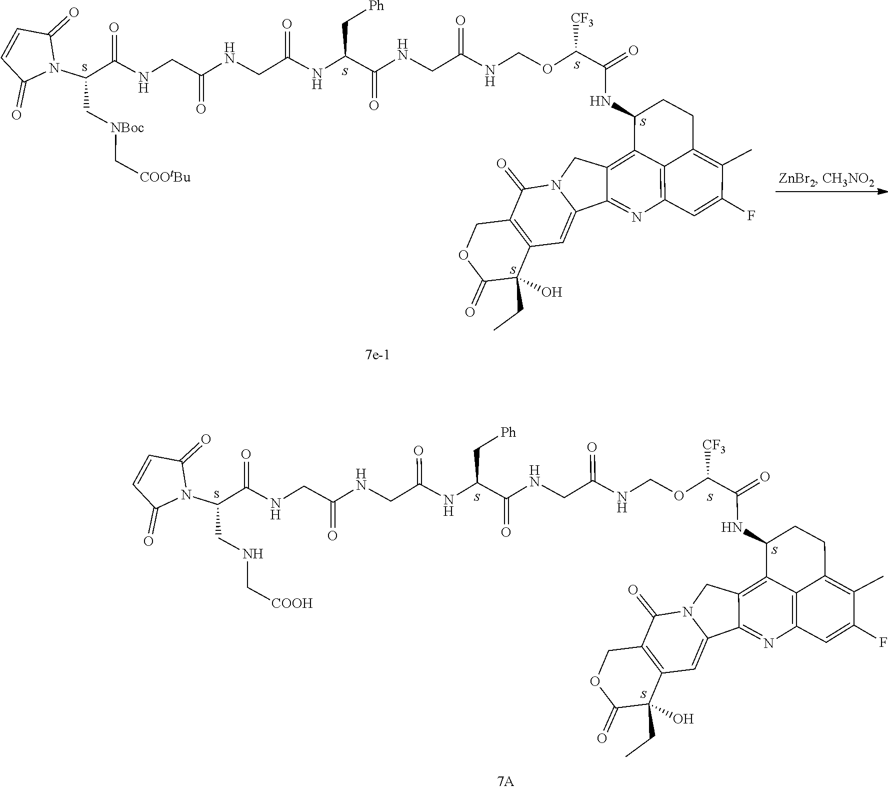

Add 7e-1 (100 mg, 0.077 mmol), zinc bromide (349 mg, 1.55 mmol) and 5 mL of nitromethane to a 25 mL single-necked flask, and react at 40° C. for 1 h. After the completion of the reaction as detected by HIPLC, the solvent was removed by concentration under reduced pressure, and a crude product was obtained. The crude product was purified by high performance liquid chromatography to obtain a product preparation liquid, and the preparation liquid was lyophilized to obtain 52 mg of solid; TOF result: 1133.3613.

### Step 6: Compound 7B

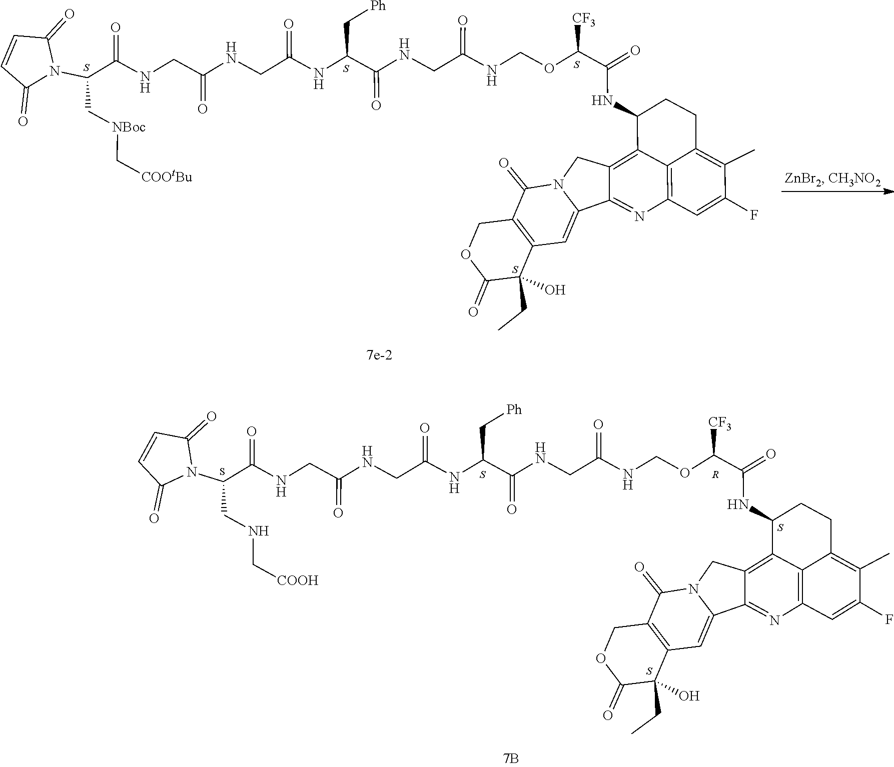

Add 7e-2 (100 mg, 0.077 mmol), zinc bromide (349 mg, 1.55 mmol) and 5 mL of nitromethane to a 25 mL single-necked flask and react at 40° C. for 1 h. After the completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain 63 mg of solid; TOF result: 1133.3668.

### Example 13

### Synthesis of Compounds 8A and 8B

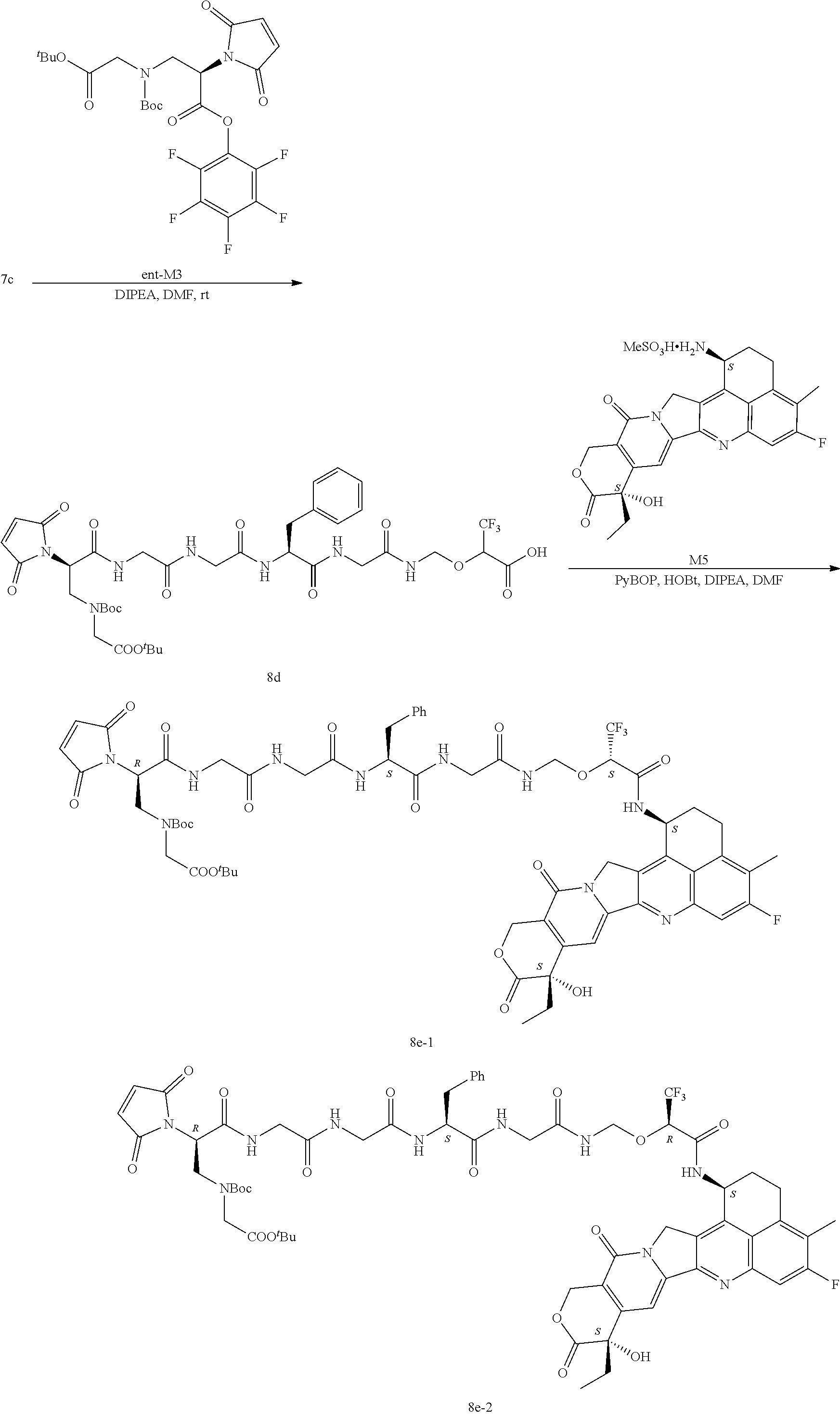

### Step 1: Compound 8d

In a 25 mL single-necked flask, 7c (900 mg, 1.83 mmol) was added, 20 mL of DMF was used to dissolve, then DIPEA (303 uL, 1.83 mmol) was added, and ent-M3 (1034 mg, 1.83 mmol) was added, and then the temperature was raised to room temperature to react for 1 h. After the completion of the reaction as detected by HPLC, the reaction solution was purified by high-performance liquid chromatography to obtain a preparation liquid, which was lyophilized to give 613 mg of a product at 38.5% yield; LC-MS: [M−H]⁻=870.32.

### Step 2: Compound 8e-1 and Compound 8e-2

Add 8d (500 mg, 0.57 mmol), M5 (305 mg, 0.57 mmol), PyBOP (448 mg, 0.86 mmol), HOBt (116 mg, 0.86 mmol) and 15 mL of DMF into a 50 mL single-necked flask, add DIPEA (378 uL, 2.29 mmol) in an ice-water bath, and raise to room temperature to react for 2 h. After the completion of the reaction as detected by HPLC, the reaction solution was purified by high performance liquid chromatography to obtain the preparation liquids of compound 8e-1 and compound 8e-2, and the preparation liquids were lyophilized to obtain 140 mg of compound 8e-1 and 210 mg of compound 8e-2, respectively. LC-MS of compound 8e-1: [M+H]⁺=1289.47; LC-MS of compound 8e-2: [M+H]⁺=1289.47.

### Step 3: Compound 8A

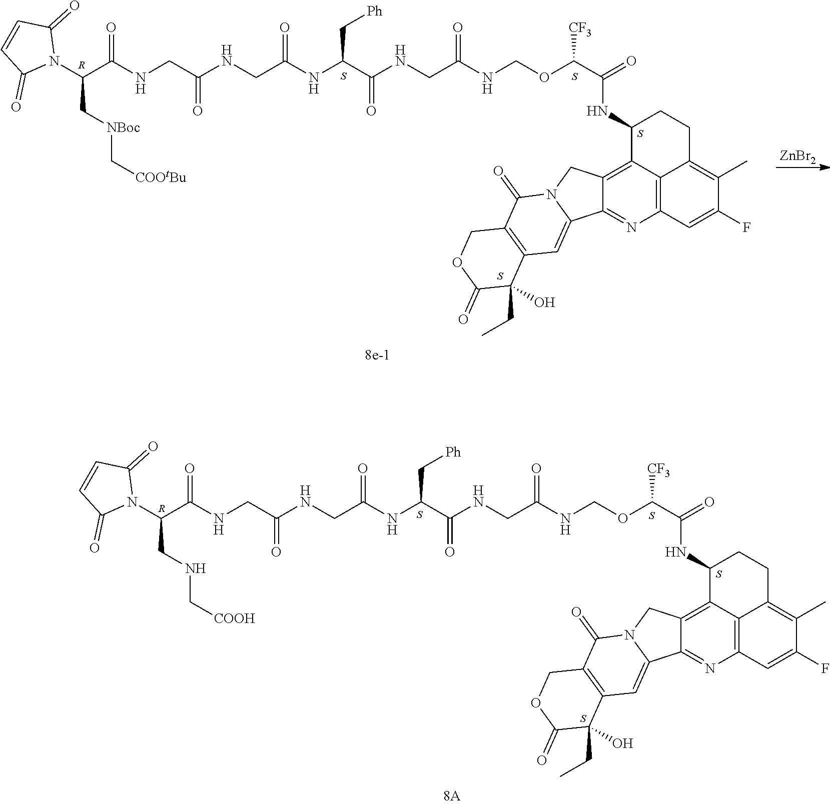

Add Compound 8e-1 (100 mg, 0.077 mmol), zinc bromide (349 mg, 1.55 mmol) and 5 mL of nitromethane into a 25 mL single-necked flask, and react at 40° C. for 1 h. After the completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain 50 mg of solid; TOF result: 1133.3623.

### Step 4: Compound 8B

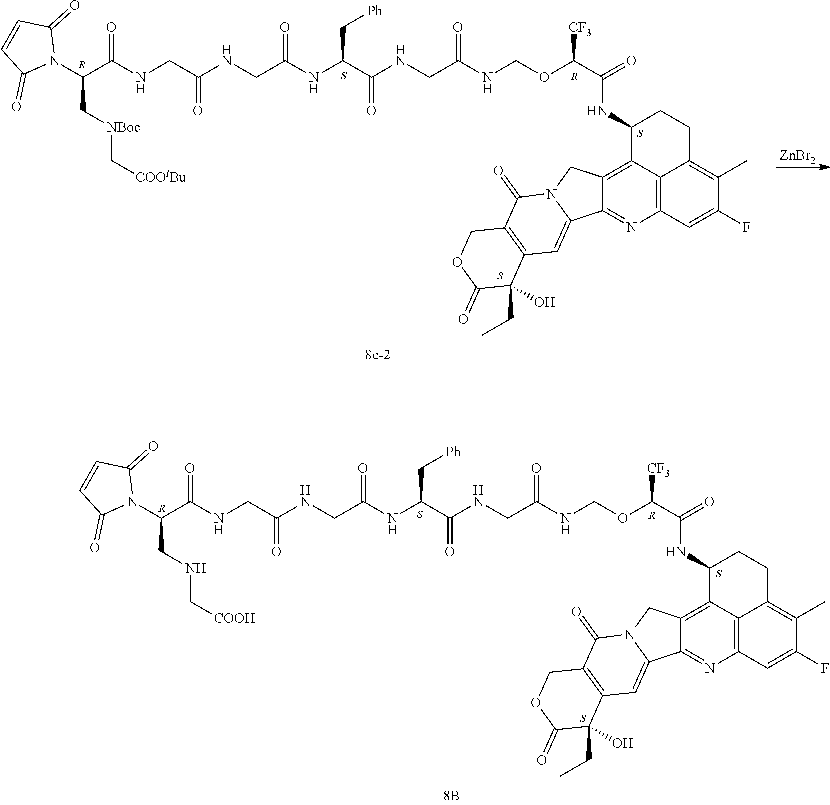

Add Compound 8e-2 (100 mg, 0.077 mmol), zinc bromide (349 mg, 1.55 mmol) and 5 mL of nitromethane into a 25 mL single-necked flask and react at 40° C. for 1 h. After the completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain 58 mg of a solid; TOF result: 1133.3653.

### Example 14

### Synthesis of Compound 9A

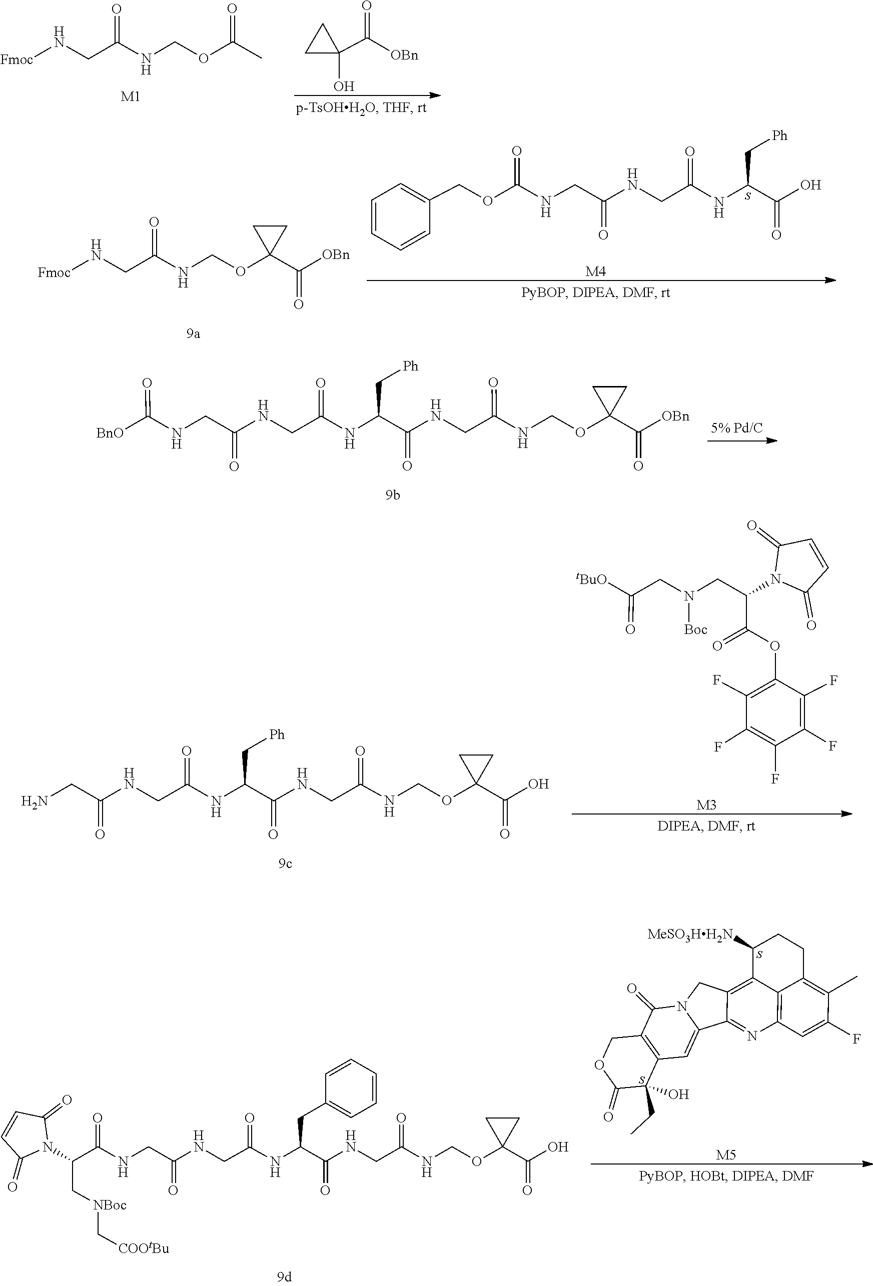

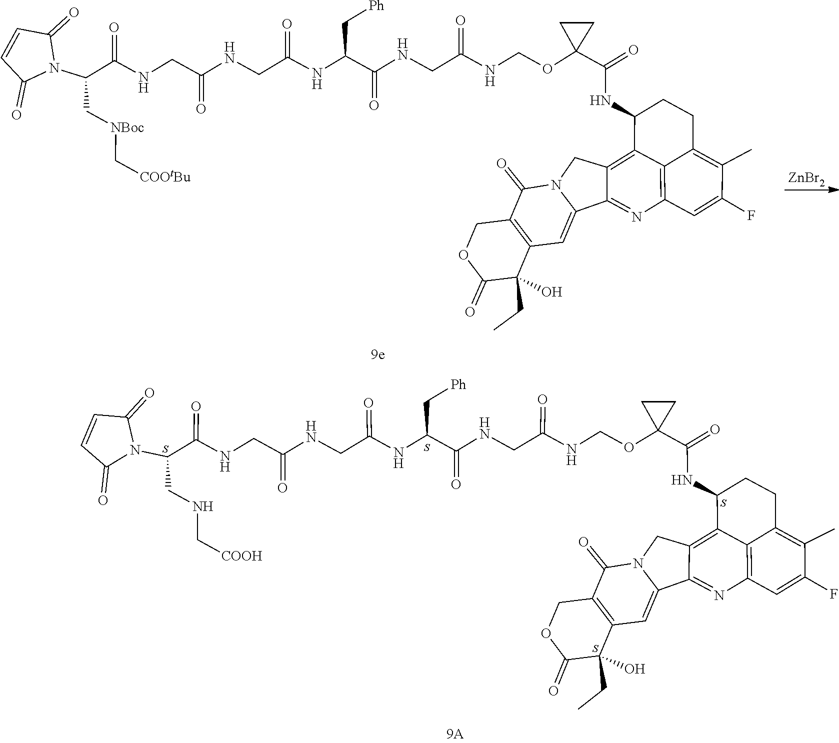

### Step 1: Compound 9a

In a 250 mL single-necked flask, add M1 (6 g, 16.3 mmol), 100 mL THF and p-toluenesulfonic acid monohydrate (0.31 g, 1.63 mmol), stir and cool to 0° C., add benzyl 2-hydroxy-2-cyclopropyl acetate dropwise (prepared according to the method published in patent US20050020645 A1) (6.3 g, 32.6 mmol), then naturally warm up to room temperature to react (the reaction lasts for about 2-4 h), with TLC monitoring. At the end of the reaction, saturated NaHCO₃ solution was added, followed by extraction with ethyl acetate, washing with saturated sodium chloride solution, drying with anhydrous sodium sulfate, filtering and concentration, and the residue was purified by silica gel column (PE:EA=10:1-5:1-2:1) to give 9a (3.7 g) at 45% yield; LC-MS: [M+H]⁺=501.5.

### Step 2: Compound 9b

In a 25 mL single-necked flask, add 9a (2 g, 4.0 mmol) and 10 mL DMF, stir at 0° C., add DBU (760 mg, 5.0 mmol), and react for 1 h. After the completion of TLC-monitored Fmoc deprotection, the reaction solution was set aside, ready for use.

Add M4 (1.65 g, 4.0 mmol), PyBOP (2.59 g, 5.0 mmol), HOBt (675 mg, 5.0 mmol) and 10 mL of DMF into another 25 mL single-necked flask, add DIPEA (823 uL, 5.04 mmol) in an ice-water bath, continue stirring for 30 min, and then add the above reaction solution to the reaction flask and raise to room temperature to react. After completion of the reaction as detected by HPLC, the reaction solution was purified by preparative LC to obtain a product preparation liquid, which was extracted by dichloromethane, washed with saturated sodium chloride solution, dried with anhydrous sodium sulfate, and filtered, and the filtrate was concentrated under reduced pressure to obtain 1.5 g of a solid at 56% yield; LC-MS: [M+H]⁺=674.7.

### Step 3: Compound 9c

9b (900 mg, 1.3 mmol) was added into a 25 mL single-necked flask, and after dissolution with 10 mL of DMF, 900 mg of 5% Pd/C was added, and a hydrogenation reaction was carried out for 1.5 h. After the completion of the reaction, filtration was performed to obtain the filtrate, which was used directly for the next step of the reaction without purification.

### Step 4: Compound 9d

Place the crude product 9c in an ice-water bath, add DIPEA (223 uL, 1.3 mmol) and then compound M3 (750 mg, 1.3 mmol), then raise to room temperature and react for 1 h. After the completion of the reaction as detected by HPLC, the reaction solution was purified by HPLC to give a preparation liquid, which was lyophilized to give 9d (529 mg); LC-MS: [M−H]⁻=828.4.

### Step 5: Compound 9e

Add 9d (500 mg, 0.6 mmol), M5 (300 mg, 0.6 mmol), PyBOP (416 mg, 0.8 mmol), HOBt (108 mg, 0.5 mmol) and 15 mL of DMF into a 50 mL single-necked flask, add DIPEA (351 uL, 2.13 mmol) in an ice-water bath, raise to room temperature and react for 3 h. After the completion of the reaction as detected by HPLC, the reaction solution was purified by high performance liquid chromatography to obtain the preparative liquid of compound 9e, which was lyophilized to obtain 9e (257 mg); LC-MS: [M+H]⁺=1247.5.

### Step 6: Compound 9A

Add 9e (100 mg, 0.08 mmol), zinc bromide (360 mg, 1.6 mmol) and 5 mL of nitromethane to a 25 mL single-necked flask and carry out a reaction at 40° C. for 1 h. After the completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain the solid compound 9A (55 mg); LC-MS: [M+H]⁺=1091.3.

### Example 15

### Synthesis of Compound 9B

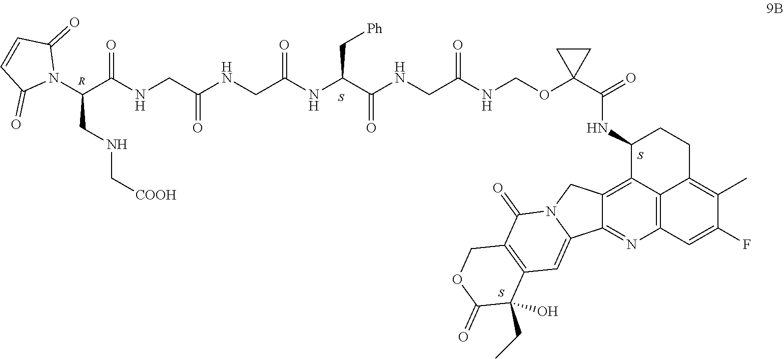

Referring to the synthetic route of Example 14, compound 9B (44 mg) was obtained; LC-MS: [M+H]⁺=1091.3.

### Example 16

### Synthesis of Compound 10A

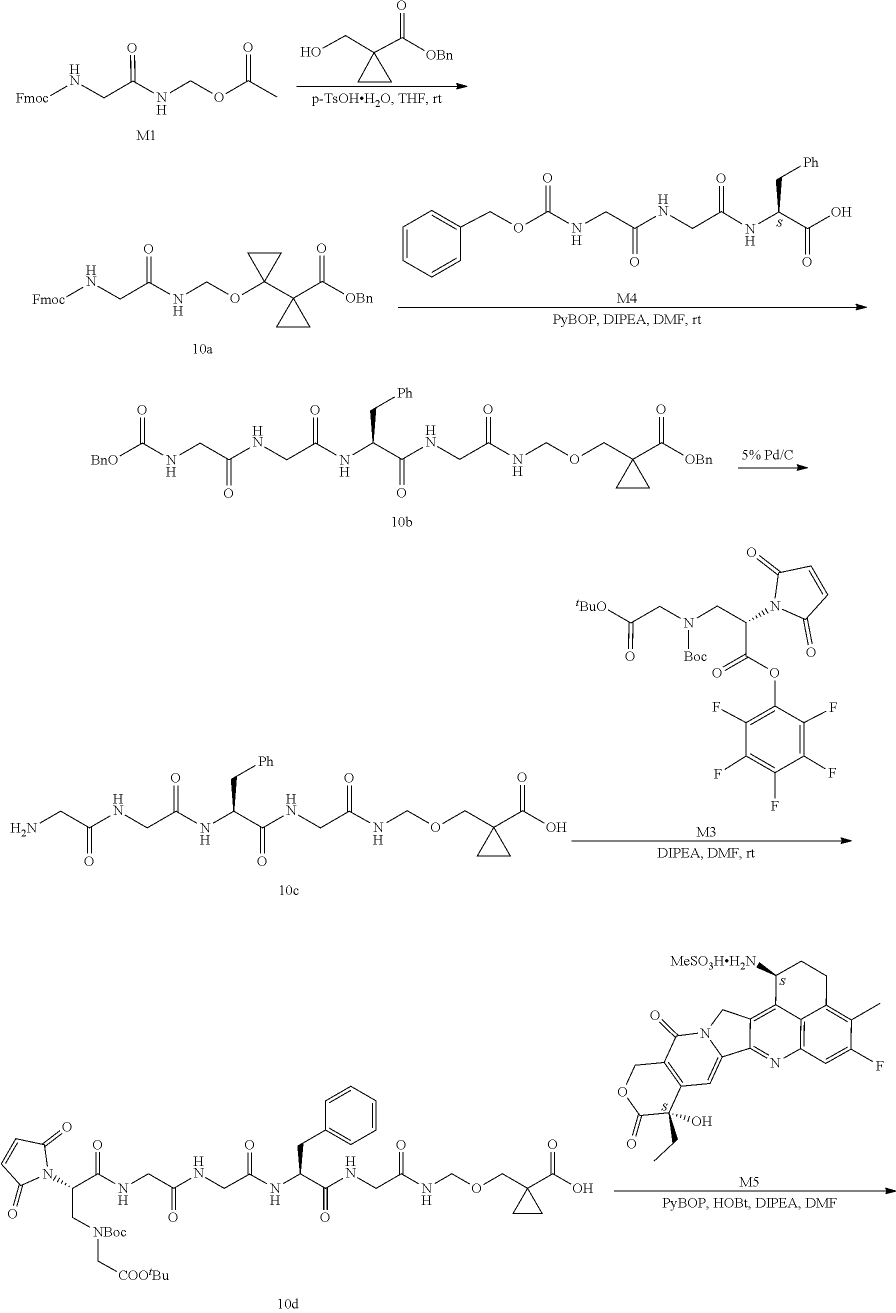

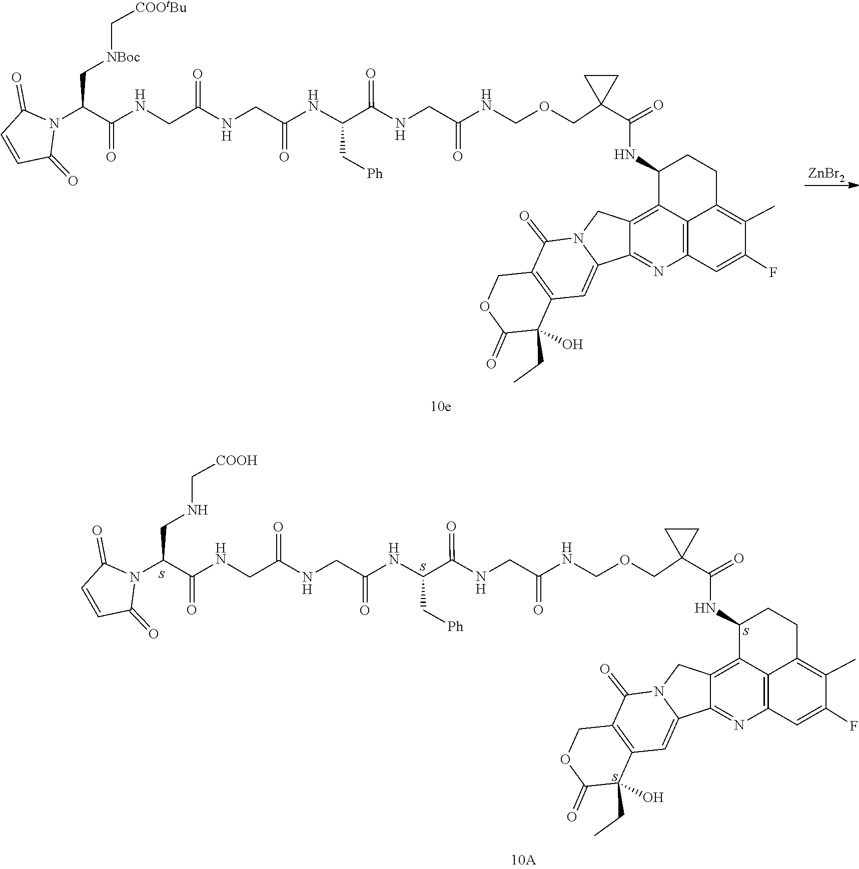

### Step 1: Compound 10a

In a 250 mL single-necked flask, add M1 (6 g, 16.3 mmol), 100 mL THF and p-toluenesulfonic acid monohydrate (0.31 g, 1.63 mmol), stir and cool to 0° C., add benzyl 3-hydroxy-2-cyclopropylpropionate dropwise (prepared with reference to the method published in patent WO2013187496A1) (6.7 g, 32.6 mmol), then naturally warm up to room temperature to react (react for about 2-4 h), with TLC monitoring. At the end of the reaction, saturated NaHCO₃ solution was added, followed by extraction with ethyl acetate, washing with saturated sodium chloride solution, drying with anhydrous sodium sulfate, filtering and concentration, and the residue was purified by silica gel column (PE:EA=10:1-5:1-2:1) to give 10a (4.9 g) at 58% yield; LC-MS: [M+H]⁺=515.4.

### Step 2: Compound 10b

In a 25 mL single-necked flask, add 10a (4 g, 7.8 mmol) and 10 mL DMF, stir at 0° C., add DBU (1.2 g, 8.0 mmol), and react for 1 h. After the completion of TLC-monitored Fmoc deprotection, set aside the reaction solution, ready for use.

Add M4 (3.3 g, 8.0 mmol), PyBOP (5.2 g, 10.0 mmol), HOBt (1.35 g, 10.0 mmol) and 10 mL of DMF into another 25 mL single-necked flask, and add DIPEA (1.65 mL, 10.1 mmol) in an ice-water bath, continue stirring for 50 min, and then add the above reaction solution to the reaction flask, raise to room temperature and react. After the completion of the reaction as detected by HIPLC, the reaction solution was purified by preparative LC to obtain a product preparation liquid, which was extracted by dichloromethane, washed with saturated sodium chloride solution, dried with anhydrous sodium sulfate and filtered, and the filtrate was concentrated under reduced pressure to obtain 2.3 g of a solid at 42% yield; LC-MS: [M+H]⁺=688.8.

### Step 3: Compound 10c

Add 10b (1.0 g, 1.45 mmol) into a 25 mL single-necked flask, dissolve in 15 mL of DMF until clear, then add 1.0 g of 5% Pd/C, and perform a hydrogenation reaction for 1.5 h. After the completion of the reaction, filtration was performed to obtain a filtrate, which was used directly for the next step of the reaction without purification.

### Step 4: Compound 10d

The crude product 10c was placed in an ice-water bath, DIPEA (258 uL, 1.5 mmol) was added, and compound M3 (837 mg, 1.45 mmol) was then added, and then the temperature was raised to room temperature to react for 1 h. Upon completion of the reaction as detected by HIPLC, the reaction solution was purified by HIPLC to obtain a preparation liquid, which was lyophilized to obtain 10d (499 mg); LC-MS: [M−H]⁻=842.4.

### Step 5: Compound 10e

10d (400 mg, 0.48 mmol), M5 (240 mg, 0.48 mmol), PyBOP (250 mg, 0.48 mmol), HOBt (104 mg, 0.48 mmol) and 15 mL of DMF were added into a 50 mL single-necked flask, DIPEA (330 uL, 2.0 mmol) was added in an ice-water bath, and the temperature was raised to room temperature to react for 3 h. After the completion of the reaction as detected by HIPLC, the reaction solution was purified by HPLC to give the preparation liquid of compound 10e, which was lyophilized to obtain 10e (188 mg); LC-MS: [M+H]⁺=1261.5.

### Step 6: Compound 10A

10e (100 mg, 0.08 mmol), zinc bromide (360 mg, 1.6 mmol) and 5 mL of nitromethane were added to a 25 mL single-necked flask and a reaction was carried out at 40° C. for 1 h. After the completion of the reaction as detected by HIPLC, the solvent was removed by concentration under reduced pressure, and a crude product was obtained. The crude product was purified by high performance liquid chromatography to obtain a product preparation liquid, and the preparation liquid was lyophilized to obtain the solid compound 10A (61 mg); LC-MS: [M+H]⁺=1105.4.

### Example 17

### Synthesis of Compound 10B

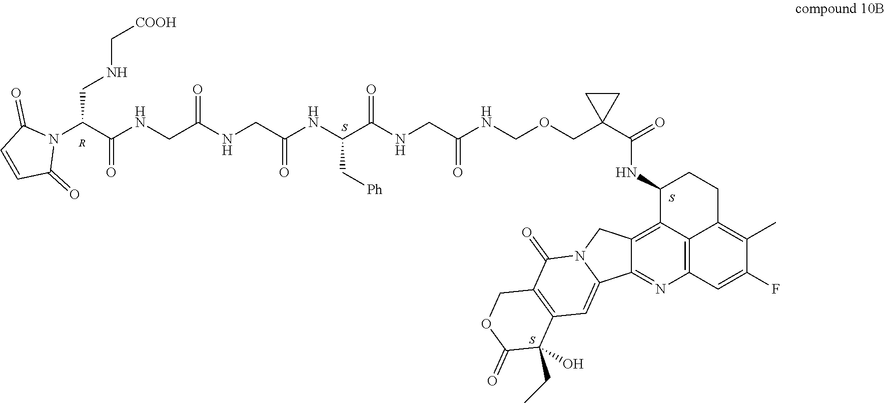

Referring to the synthetic route of Example 16, compound 10B (75 mg) was obtained; LC-MS: [M+H]⁺=1105.4.

### Example 18

### Synthesis of Compound 11A

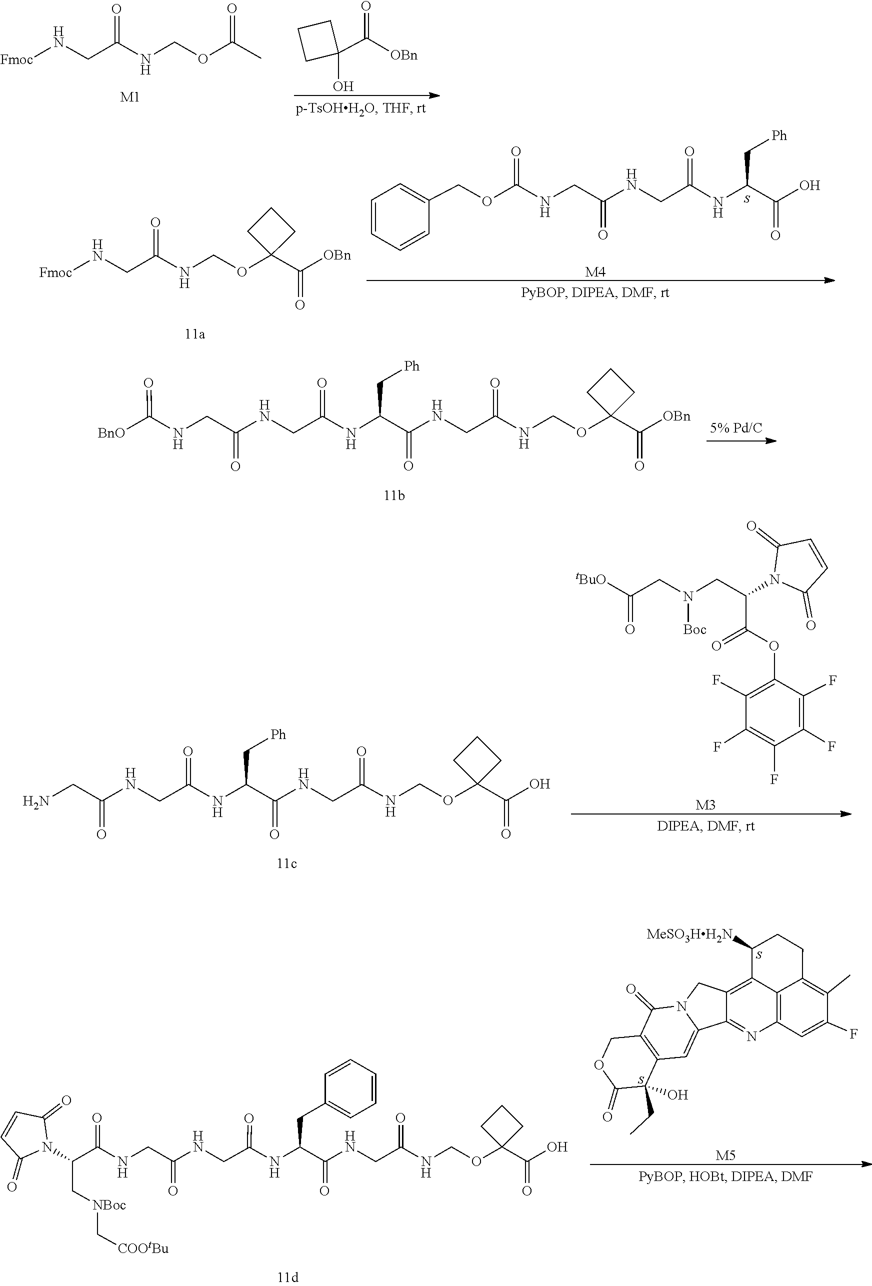

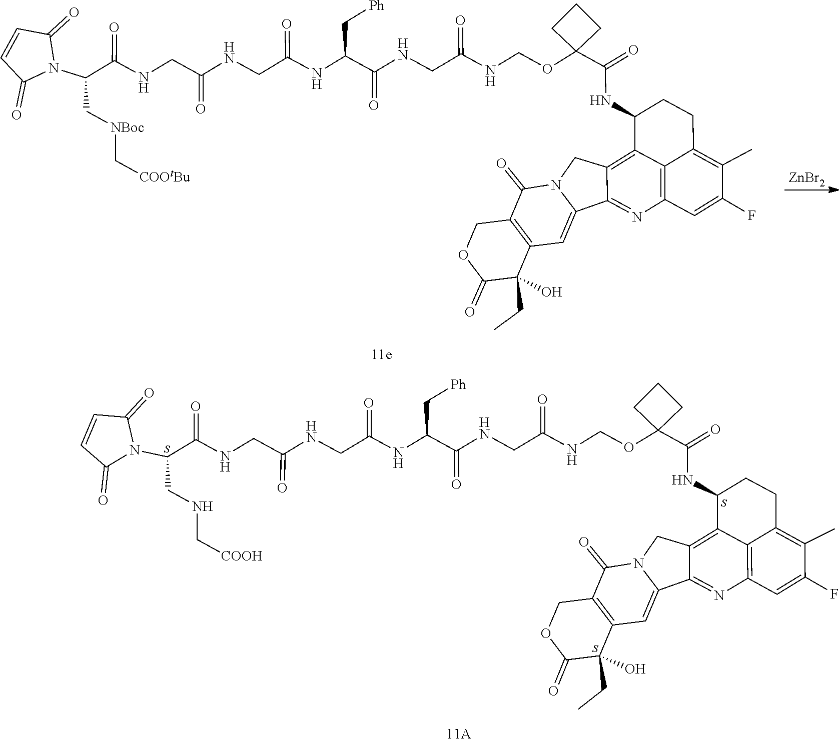

### Step 1: Compound 11a

In a 250 mL single-necked flask, add M1 (6 g, 16.3 mmol), 100 mL THF and p-toluenesulfonic acid monohydrate (0.31 g, 1.63 mmol), stir and cool to 0° C., add benzyl 2-hydroxy-2-cyclobutylacetate dropwise (synthesized by the method published in the literature Journal of Medicinal Chemistry, 2013, 56(13), 5541-5552) (6.7 g, 32.6 mmol), then naturally warm up to room temperature and react (react for about 2-4 h), with TLC monitoring. At the end of the reaction, saturated NaHCO₃ solution was added, followed by extraction with ethyl acetate, washing with saturated sodium chloride solution, drying with anhydrous sodium sulfate, filtering and concentration, and the residue was purified by silica gel column (PE:EA=10:1-5:1-2:1) to give 11a (5.1 g) at 62% yield; LC-MS: [M+H]⁺=515.7.

### Step 2: Compound 11b

In a 25 mL single-necked flask, add 11a (4 g, 7.8 mmol) and 10 mL DMF, stir at 0° C., add DBU (1.2 g, 8.0 mmol), and react for 1 h. After the completion of TLC-monitored Fmoc deprotection, set aside the reaction solution, ready for use.

Add M4 (3.3 g, 8.0 mmol), PyBOP (5.2 g, 10.0 mmol), HOBt (1.35 g, 10.0 mmol) and 10 mL of DMF into another 25 mL single-necked flask, and add DIPEA (1.63 mL, 10.0 mmol) in an ice-water bath, continue stirring for 40 min, and then add the above reaction solution to the reaction flask, and raise to room temperature to react. After the completion of the reaction as detected by HPLC, the reaction solution was purified by preparative LC to obtain a product preparation liquid, which was extracted by dichloromethane, washed with saturated sodium chloride solution, dried with anhydrous sodium sulfate and filtered, and the filtrate was concentrated under reduced pressure to obtain 2.3 g of a solid at 42% yield; LC-MS: [M+H]⁺=688.3.

### Step 3: Compound 11c

Add 11b (2.0 g, 2.9 mmol) into a 25 mL single-necked flask, and after dissolution with 25 mL of DMF, add 2.0 g of 5% Pd/C, and carry out a hydrogenation reaction for 3 h. After the completion of the reaction, it was filtered to obtain a filtrate, which was used directly for the next step of the reaction without purification.

### Step 4: Compound 11d

The crude product 11c was placed in an ice-water bath, DIPEA (516 uL, 3.0 mmol) was added, and then compound M3 (1.7 g, 2.9 mmol) was added, and then the temperature was raised to room temperature to react for 2 h. After the completion of the reaction as detected by HPLC, the reaction solution was purified by HPLC to obtain a preparation liquid, which was lyophilized to give 11d (934 mg); LC-MS: [M−H]⁻=842.4.

### Step 5: Compound 11e

Add 11d (800 mg, 0.96 mmol), M5 (480 mg, 0.96 mmol), PyBOP (500 mg, 0.96 mmol), HOBt (208 mg, 0.96 mmol) and 30 mL of DMF into a 50 mL single-necked flask, add DIPEA (660 uL, 4.0 mmol) in an ice-water bath, raise to room temperature and react for 4 h. After the completion of the reaction as detected by HPLC, the reaction solution was purified by HPLC to give the preparation liquid of compound 11e, which was lyophilized to obtain 11e (401 mg); LC-MS: [M+H]⁺=1261.4.

### Step 6: Compound 11A

11e (150 mg, 0.12 mmol), zinc bromide (532 mg, 2.4 mmol) and 10 mL of nitromethane were added to a 25 mL single-necked flask and a reaction was carried out at 40° C. for 1 h. After the reaction was complete as detected by HPLC, the solvent was removed by concentration under reduced pressure, and a crude product was obtained. The crude product was purified by high performance liquid chromatography to obtain a product preparation liquid, and the preparation liquid was lyophilized to obtain the solid compound 11A (86 mg); LC-MS: [M+H]⁺=1105.4.

### Example 19

### Synthesis of Compound 11B

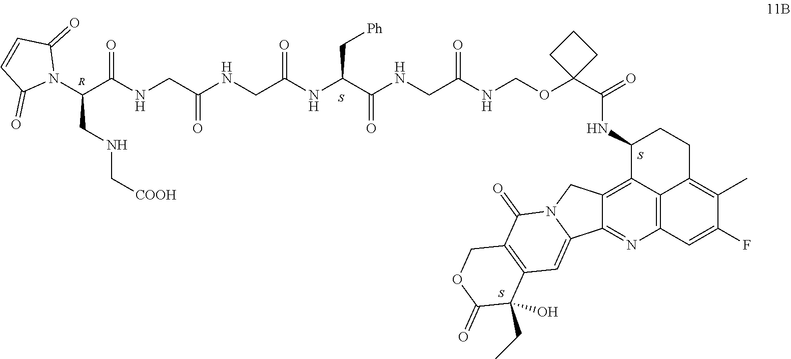

Referring to the synthetic route of Example 18, compound 11B (50 mg) was obtained. LC-MS: [M+H]⁺=1105.4.

### Example 20

### Synthesis of Compound 12A

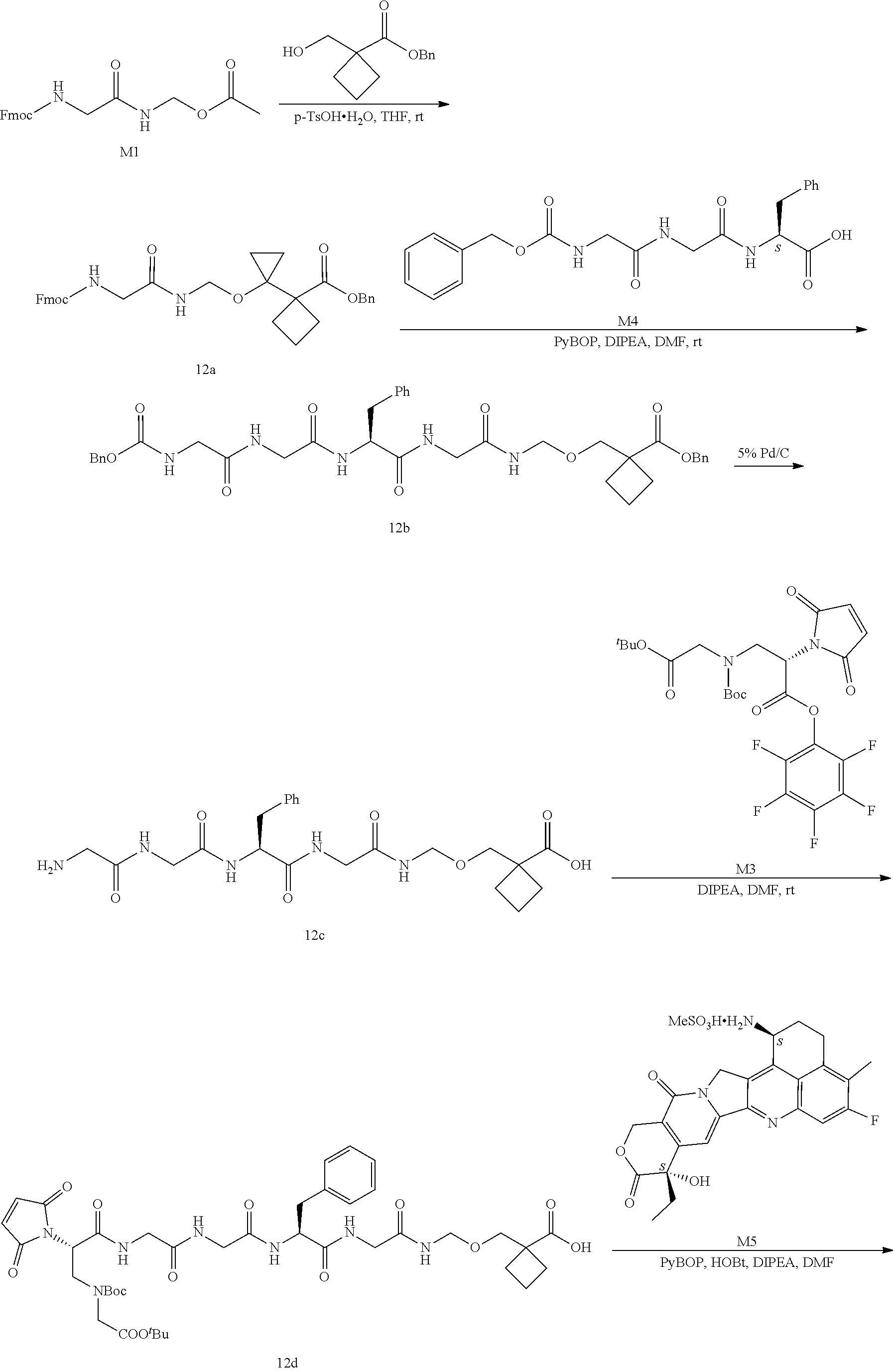

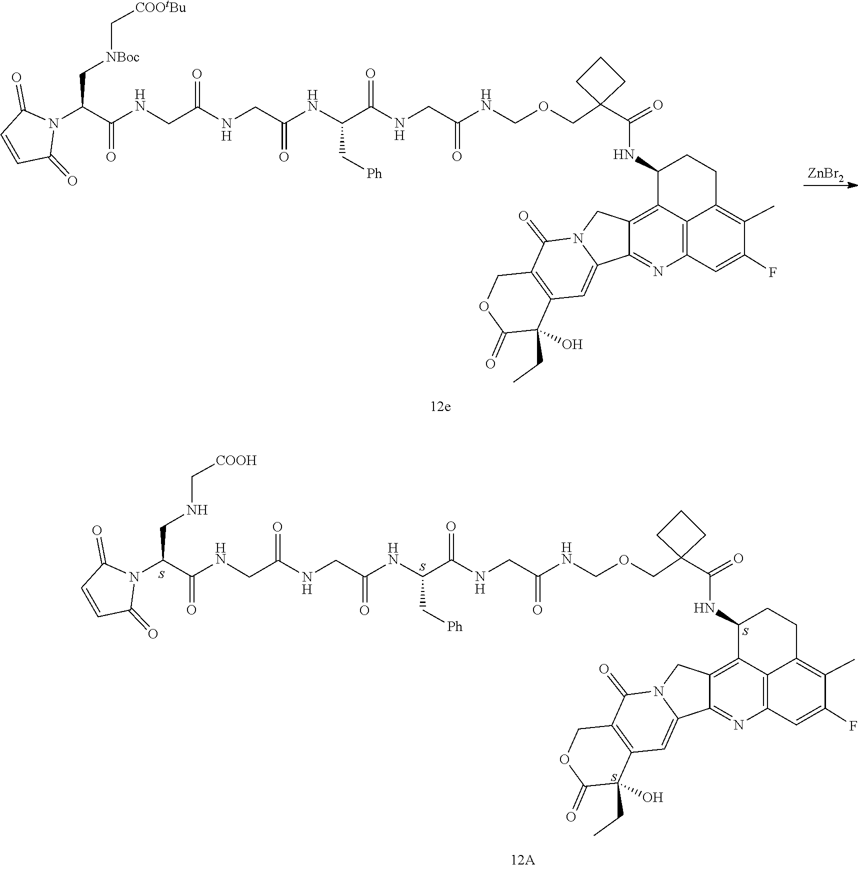

### Step 1: Compound 12a

In a 250 mL single-necked flask, add M1 (6 g, 16.3 mmol), 100 mL THF and p-toluenesulfonic acid monohydrate (0.31 g, 1.63 mmol), stir and cool to 0° C., add benzyl 3-hydroxy-2-cyclobutylpropionate dropwise (prepared according to the method published in patent WO2009011285A1) (7.2 g, 32.6 mmol). Then warm up to room temperature naturally to react (react for about 2-4 h), with TLC monitoring. At the end of the reaction, saturated NaHCO₃ solution was added, followed by extraction with ethyl acetate, washing with saturated sodium chloride solution, drying with anhydrous sodium sulfate, filtering and concentration, and the residue was purified by silica gel column (PE:EA=10:1-5:1-2:1) to give 12a (4.5 g) at 52% yield; LC-MS: [M+H]⁺=529.4.

### Step 2: Compound 12b

In a 25 mL single-necked flask, add 12a (4 g, 7.6 mmol) and 10 mL DMF, stir at 0° C., add DBU (1.2 g, 8.0 mmol), and react for 1 h. After the completion of TLC-monitored Fmoc deprotection, set aside the reaction solution, ready for use.

Add M4 (3.2 g, 7.6 mmol), PyBOP (4.7 g, 9.0 mmol), HOBt (1.22 g, 9.0 mmol) and 10 mL of DMF in another 25 mL single-necked flask, add DIPEA (1.49 mL, 0.9 mmol) in an ice-water bath, and continue stirring for 30 min, then add the above reaction solution to the reaction flask and raise the temperature to room temperature to react. After the completion of the reaction as detected by HPLC, the reaction solution was purified by preparative LC to obtain a product preparation liquid, which was extracted by dichloromethane, washed with saturated sodium chloride solution, dried with anhydrous sodium sulfate and filtered, and the filtrate was concentrated under reduced pressure to obtain 2.0 g of a solid at 37% yield; LC-MS: [M+H]⁺=702.8.

### Step 3: Compound 12c

Add 12b (1.0 g, 1.43 mmol) into a 25 mL single-necked flask, and dissolve in 15 mL of DMF until clear, add 1.0 g of 5% Pd/C, and carry out a hydrogenation reaction for 1.5 h. After the completion of the reaction, it was filtered to obtain a filtrate, which was used directly for the next step of the reaction without purification.

### Step 4: Compound 12d

Place the crude product 12c in an ice-water bath, add DIPEA (258 uL, 1.5 mmol) and then compound M3 (825 mg, 1.43 mmol), the raise to room temperature and react for 1 h. After the completion of the reaction as detected by HPLC, the reaction solution was purified by HPLC to obtain a preparation liquid, which was lyophilized to obtain 12d (522 mg); LC-MS: [M−H]⁻=856.4.

### Step 5: Compound 12e

Add 12d (400 mg, 0.47 mmol), M5 (240 mg, 0.47 mmol), PyBOP (250 mg, 0.47 mmol), HOBt (101 mg, 0.47 mmol) and 15 mL of DMF into a 50 mL single-necked flask, add DIPEA (330 uL, 2.0 mmol) in an ice-water bath, raise to room temperature and react for 3 h. After the completion of the reaction as detected by HPLC, the reaction solution was purified by HPLC to give a preparation liquid of compound 12e, which was lyophilized to obtain 12e (198 mg); LC-MS: [M+H]⁺=1275.4.

### Step 6: Compound 12A

Add 12e (100 mg, 0.08 mmol), zinc bromide (360 mg, 1.6 mmol) and 5 mL of nitromethane to a 25 mL single-necked flask and carry out a reaction at 40° C. for 1 h. After the completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain the solid compound 12A (55 mg); LC-MS: [M+H]⁺=1119.4.

### Example 21

### Synthesis of Compound 12B

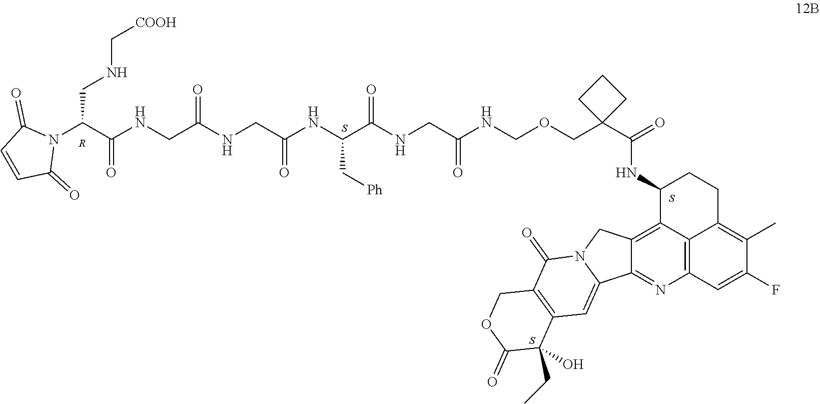

Referring to the synthetic route of Example 20, compound 12B (50 mg) was obtained; LC-MS: [M+H]⁺=1119.4.

### Example 22

### Synthesis of Compound 13A

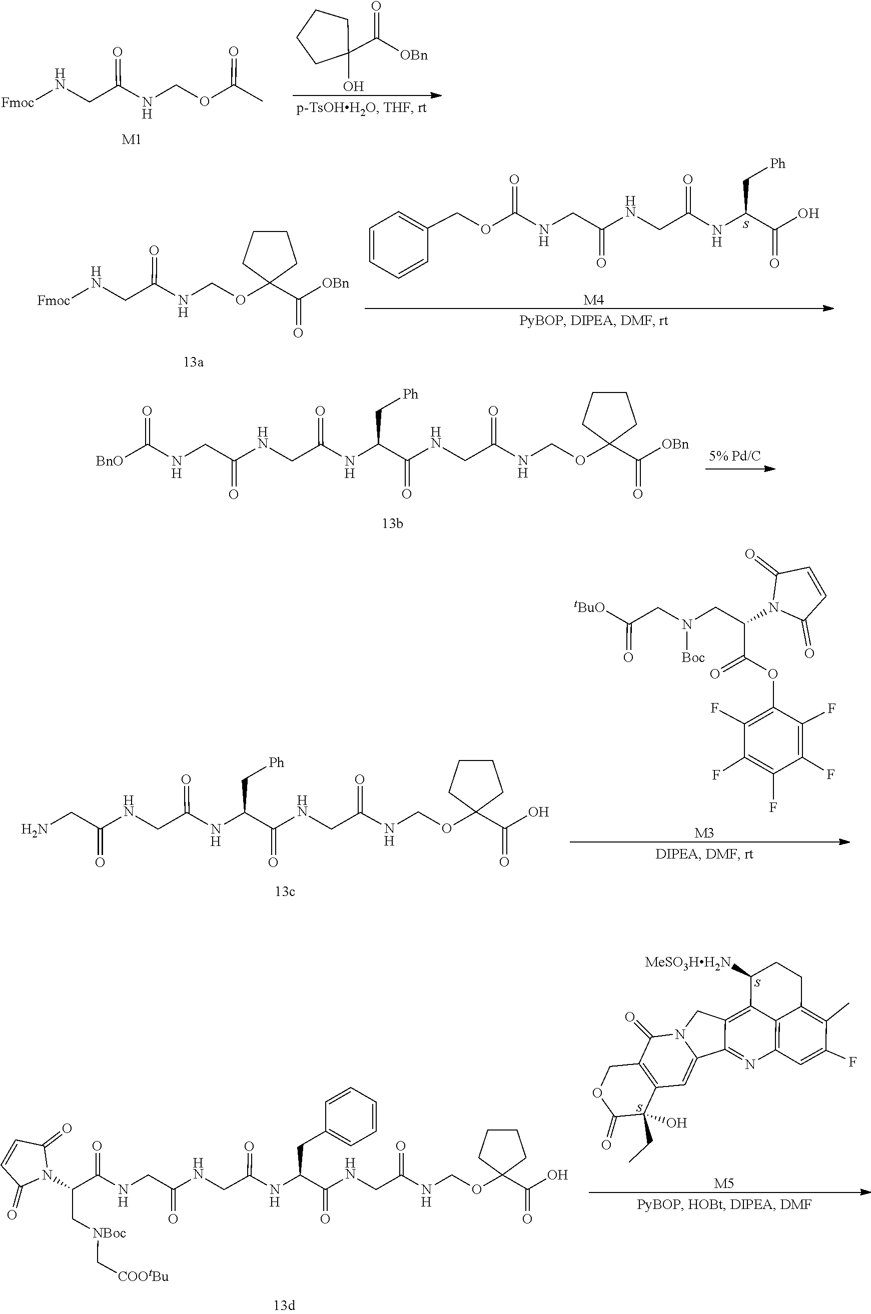

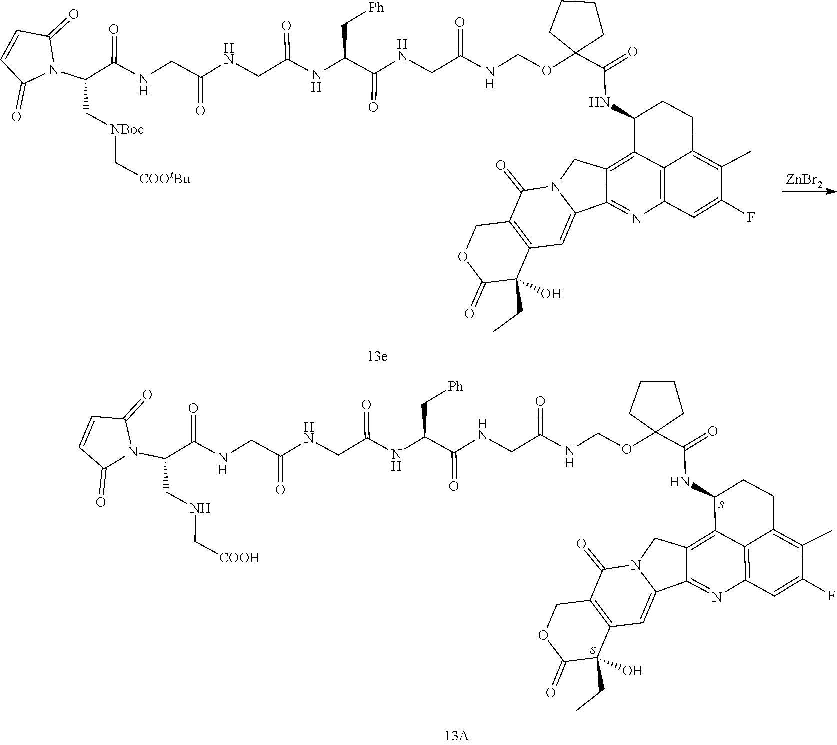

### Step 1: Compound 13a

In a 250 mL single-necked flask, add M1 (6 g, 16.3 mmol), 100 mL THF and p-toluenesulfonic acid monohydrate (0.31 g, 1.63 mmol), stir and cool to 0° C., add benzyl 2-hydroxy-2-cyclopentylacetate dropwise (synthesized by the method published in the literature, Journal of Medicinal Chemistry, 2013, 56(13), 5541-5552) (7.2 g, 32.6 mmol), then naturally warm up to room temperature to react (react for about 2-4 h), with TLC monitoring. At the end of the reaction, saturated NaHCO₃ solution was added, followed by extraction with ethyl acetate, washing with saturated sodium chloride solution, drying with anhydrous sodium sulfate, filtering and concentration, and the residue was purified by silica gel column (PE:EA=10:1-5:1-2:1) to give 13a (4.6 g) at 53% yield; LC-MS: [M+H]⁺=529.5.

### Step 2: Compound 13b

In a 25 mL single-necked flask, add 13a (4 g, 7.6 mmol) and 10 mL DMF, stir at 0° C., add DBU (1.17 g, 7.8 mmol), and react for 1 h. After the completion of TLC-monitored Fmoc deprotection, set aside the reaction solution, ready for use.

Add M4 (3.14 g, 7.6 mmol), PyBOP (4.42 g, 8.5 mmol), HOBt (1.15 g, 8.5 mmol) and 10 mL of DMF into another 25 mL single-necked flask, add DIPEA (1.39 mL, 0.85 mmol) in an ice-water bath, continue stirring for 30 min, and then add the above reaction solution to the reaction flask, and raise to room temperature to react. After the completion of the reaction as detected by HPLC, the reaction solution was purified by preparative LC to obtain a product preparation liquid, which was extracted by dichloromethane, washed with saturated sodium chloride solution, dried with anhydrous sodium sulfate and filtered, and the filtrate was concentrated under reduced pressure to obtain 2.1 g of a solid at 39% yield; LC-MS: [M+H]⁺=702.8.

### Step 3: Compound 13c

13b (1.5 g, 1.87 mmol) was added to a 25 mL single-necked flask, and after dissolution with 25 mL of DMF, 1.5 g of 5% Pd/C was added, and a hydrogenation reaction was carried out for 3 h. After completion of the reaction, filtration was performed, and a filtrate was obtained, and used directly for the next step of the reaction without purification.

### Step 4: Compound 13d

The crude product 13c was placed in an ice-water bath, DIPEA (333 uL, 1.93 mmol) was added, and compound M3 (1.1 g, 1.87 mmol) was then added, before raising to room temperature to react for 1 h. Upon completion of the reaction as detected by HPLC, the reaction solution was purified by HPLC to obtain a preparation liquid, which was lyophilized to give 13d (519 mg); LC-MS: [M−H]⁻=856.6.

### Step 5: Compound 13e

Add 13d (400 mg, 0.47 mmol), M5 (240 mg, 0.48 mmol), PyBOP (250 mg, 0.48 mmol), HOBt (103 mg, 48 mmol) and 15 mL of DMF to a 50 mL single-necked flask, add DIPEA (330 uL, 2.0 mmol) in an ice-water bath, and raise to room temperature to react for 4 h. Upon completion of the reaction as detected by HIPLC, the reaction solution was purified by HIPLC to obtain the preparation liquid of compound 13e, which was lyophilized to obtain 13e (187 mg); LC-MS: [M+H]⁺=1275.5.

### Step 6: Compound 13A

Add 13e (100 mg, 0.08 mmol), zinc bromide (355 mg, 0.16 mmol) and 5 mL of nitromethane into a 25 mL single-necked flask and carry out a reaction at 40° C. for 1 h. Upon completion of the reaction as detected by HIPLC, the solvent was removed by concentration under reduced pressure, and a crude product was obtained. The crude product was purified by high performance liquid chromatography to obtain a product preparation liquid, and the preparation liquid was lyophilized to obtain the solid compound 13A (60 mg); LC-MS: [M+H]⁺=1119.6.

### Example 23

### Synthesis of Compound 13B

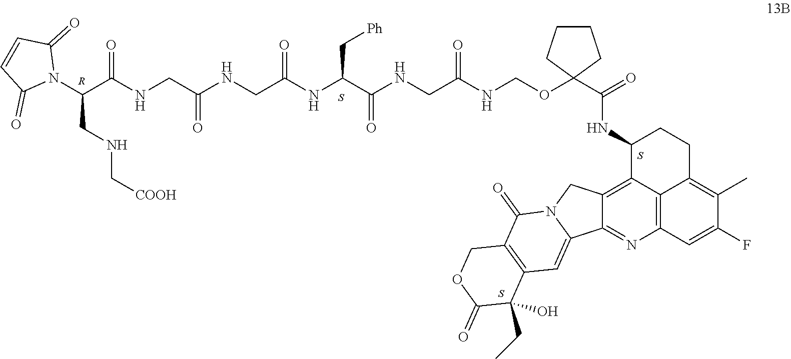

Referring to the synthetic route of Example 22, compound 13B (51 mg) was obtained; LC-MS: [M+H]⁺=1119.6.

### Example 24

### Synthesis of Compound 14A

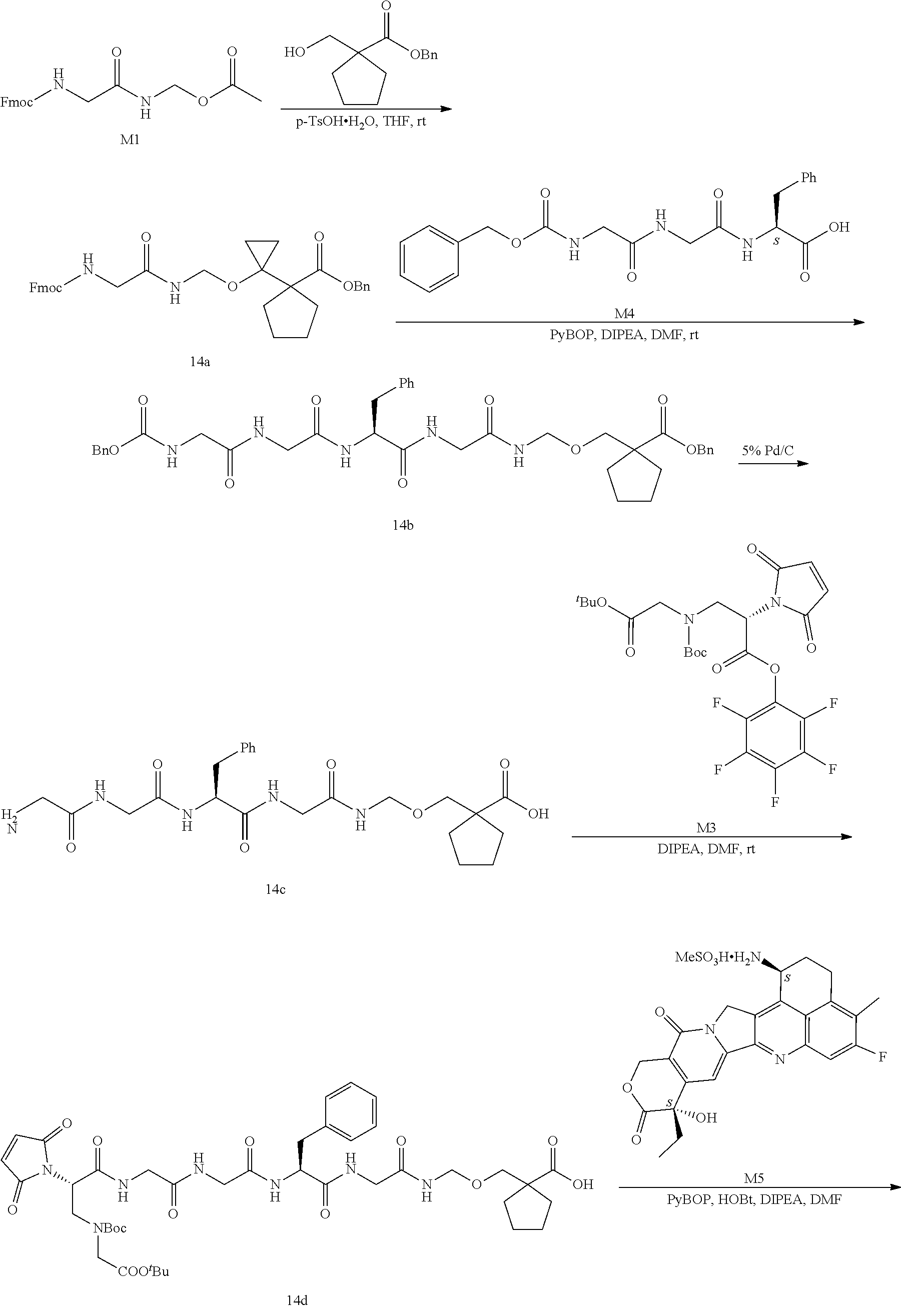

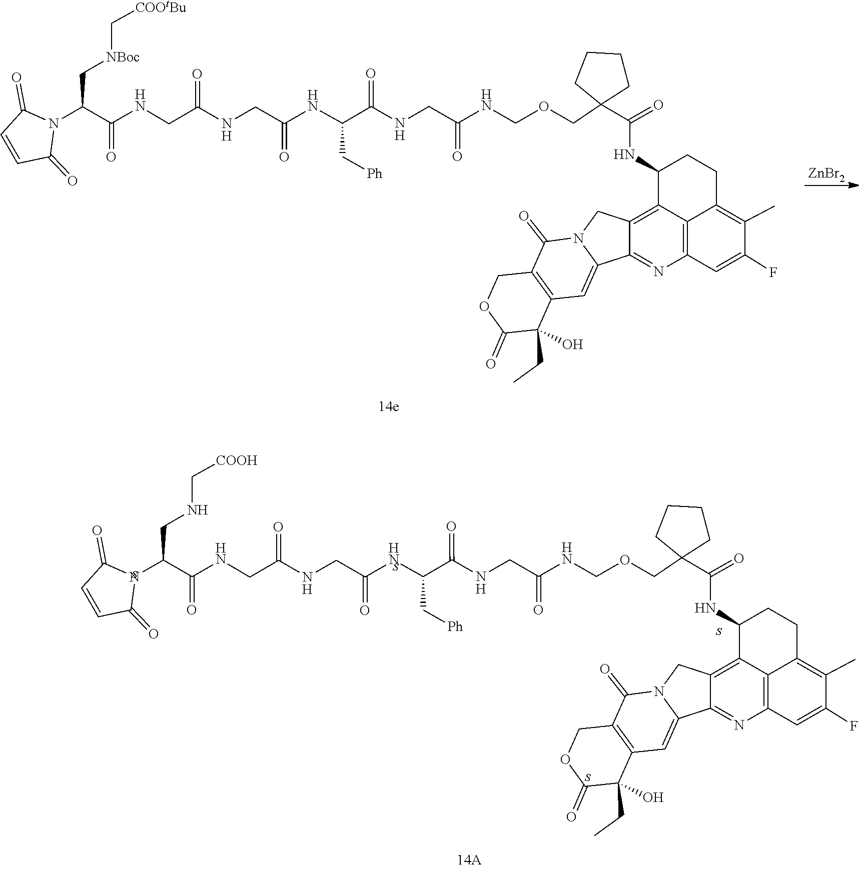

### Step 1: Compound 14a

In a 250 mL single-necked flask, add M1 (6 g, 16.3 mmol), 100 mL THF and p-toluenesulfonic acid monohydrate (0.31 g, 1.63 mmol), stir and cool to 0° C., add benzyl 3-hydroxy-2-cyclopentylpropionate dropwise (synthesized by the method published in patent WO2009011285A1) (7.6 g, 32.6 mmol), then naturally warm up to room temperature and react (react for about 2-4 h), with TLC monitoring. At the end of the reaction, saturated NaHCO₃ solution was added, followed by extraction with ethyl acetate, washing with saturated sodium chloride solution, drying with anhydrous sodium sulfate, filtering and concentration, and the residue was purified by silica gel column (PE:EA=10:1-5:1-2:1) to give 14a (4.4 g) at 49% yield; LC-MS: [M+H]⁺=543.6.

### Step 2: Compound 14b

In a 25 mL single-necked flask, add 14a (4 g, 7.4 mmol) and 10 mL DMF, stir at 0° C., add DBU (1.2 g, 8.0 mmol), and react for 1 h. After the completion of Fmoc deprotection as detected by TLC, set aside the reaction solution, ready for use.

Add M4 (3.1 g, 7.4 mmol), PyBOP (4.6 g, 8.8 mmol), HOBt (1.19 g, 8.8 mmol) and 10 mL of DMF into another 25 mL single-necked flask, add DIPEA (1.49 mL, 9.0 mmol) in an ice-water bath, and continue stirring for 30 min, then add the above reaction solution to the reaction flask and raise the temperature to room temperature to react. After the completion of the reaction as detected by HPLC, the reaction solution was purified by preparative LC to obtain a product preparation liquid, which was extracted by dichloromethane, washed with saturated sodium chloride solution, dried with anhydrous sodium sulfate and filtered, and the filtrate was concentrated under reduced pressure to obtain 2.6 g of a solid at 49% yield; LC-MS: [M+H]⁺=716.4.

### Step 3: Compound 14c

In a 25 mL single-necked flask, 14b (1.0 g, 1.4 mmol) was added, and after dissolution with 15 mL of DMF, 1.0 g of 5% Pd/C was added, and a hydrogenation reaction was carried out for 1.5 h. After completion of the reaction, filtration was performed, and a filtrate was obtained, and used directly for the next step of the reaction without purification.

### Step 4: Compound 14d

The crude product 14c was placed in an ice-water bath, DIPEA (248 uL, 1.5 mmol) was added, then compound M3 (808 mg, 1.4 mmol) was added, then the temperature was raised to room temperature to react for 1 h. When the reaction was completed as detected by HPLC, the reaction solution was purified by HPLC to obtain a preparation liquid, which was lyophilized to obtain 14d (500 mg); LC-MS: [M−H]⁻=870.5.

### Step 5: Compound 14e

Add 14d (400 mg, 0.46 mmol), M5 (235 mg, 0.46 mmol), PyBOP (245 mg, 0.46 mmol), HOBt (99 mg, 0.46 mmol) and 15 mL of DMF into a 50 mL single-necked flask, add DIPEA (331 uL, 2.0 mmol) in an ice-water bath, raise to room temperature and react for 3 h. After the completion of the reaction as detected by HPLC, the reaction solution was purified by HPLC to give the preparation liquid of compound 14e, which was lyophilized to obtain 14e (146 mg); LC-MS: [M+H]⁺=1289.5.

### Step 6: Compound 14A

14e (100 mg, 0.08 mmol), zinc bromide (360 mg, 1.6 mmol) and 5 mL of nitromethane were added to a 25 mL single-necked flask and a reaction was carried out at 40° C. for 1 h. After completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain the solid compound 14A (52 mg); LC-MS: [M+H]⁺=1133.4.

### Example 25

### Synthesis of Compound 14B

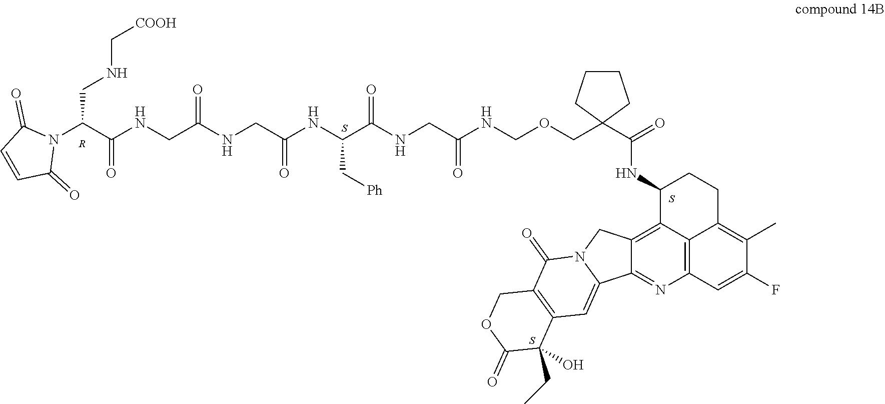

Referring to the synthetic route of Example 24, compound 14B (48 mg) was obtained; LC-MS: [M+H]⁺=1133.4.

### Example 26

### Synthesis of Compounds 15A and 15B

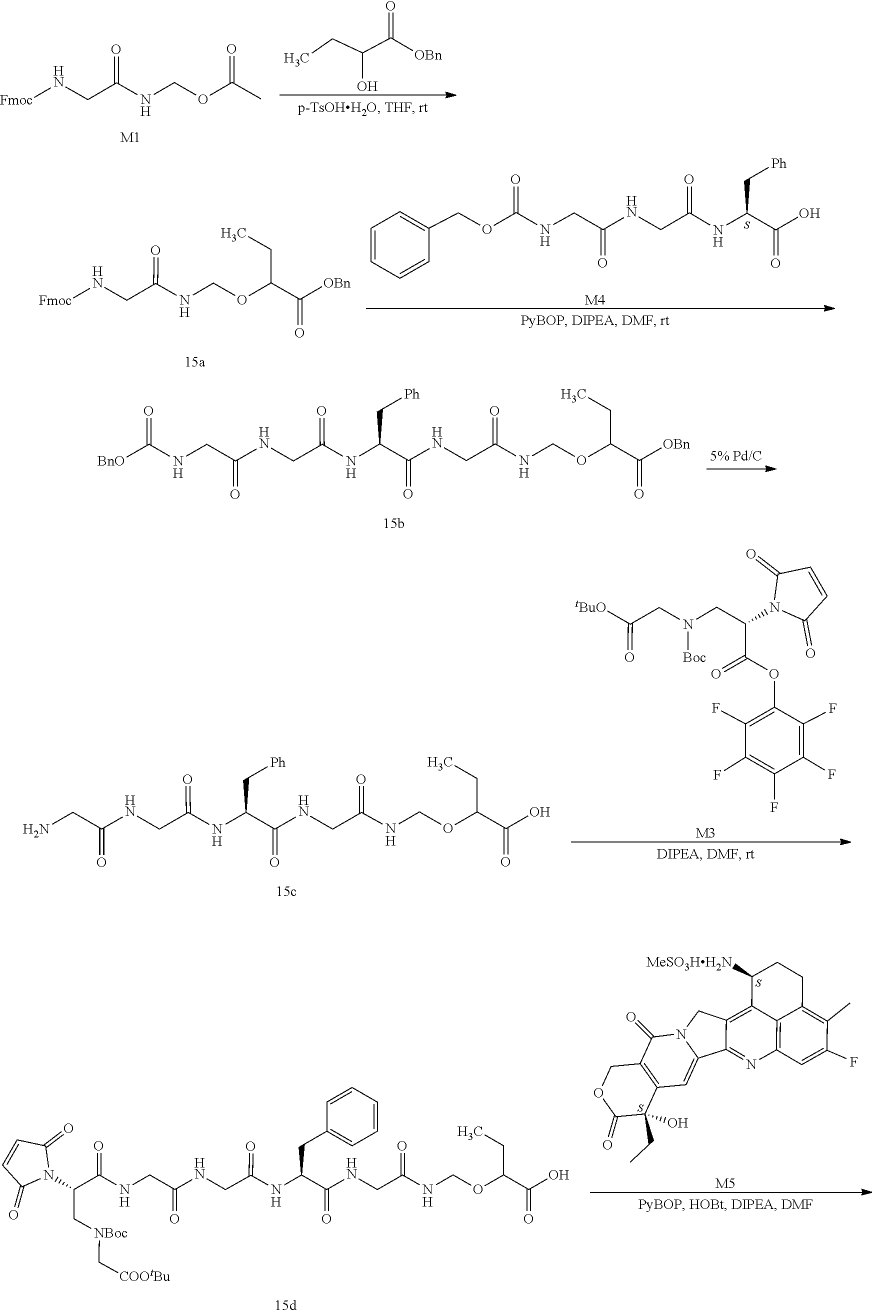

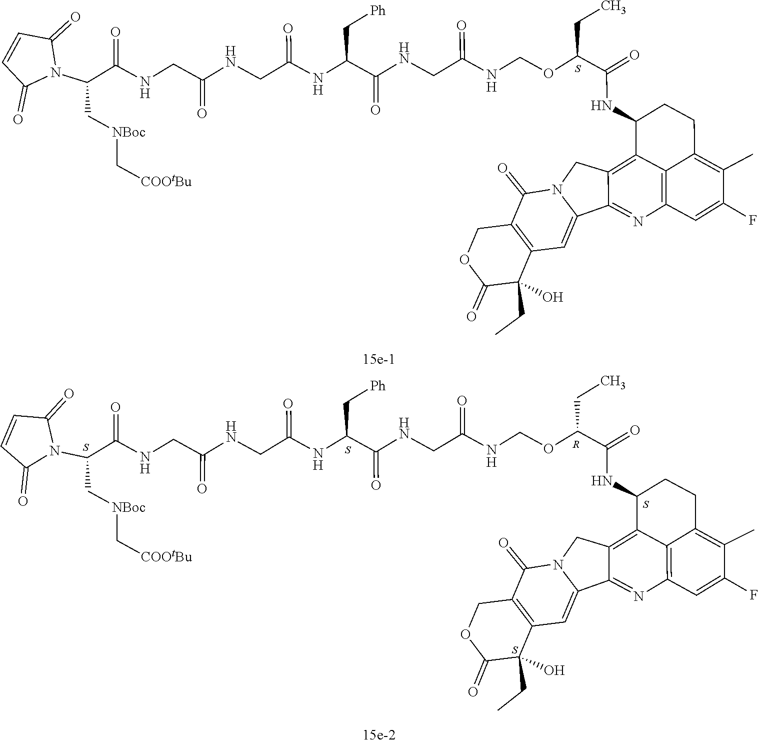

### Step 1: Compound 15a

In a 250 mL single-necked flask, M1 (10 g, 27.1 mmol), benzyl 2-hydroxy-butyrate (prepared by the method published in the literature Chemical Communications, 2019, 55(53), 7699-7702) (10.5 g, 54.3 mmol), zinc acetate (9.96 g, 54.3 mmol) and 100 mL of toluene were added, heated to 100° C. and reacted for 4 h. After the reaction was completed, the temperature was reduced to room temperature, filtration was performed to remove insoluble material, and the filtrate was concentrated to obtain a crude product. The crude product was purified by silica gel column chromatography (PE:EA=10:1-5:1-2:1) to obtain 5.67 g of the target material at 42% yield; LC-MS: [M+H]⁺=503.5.

### Step 2: Compound 15b

Add 15a (5 g, 9.95 mmol) and 15 mL of DMF in a 50 mL single-necked flask, and after dissolution, add DBU (1.68 g, 11 mmol) in an ice-water bath and react for 1 h. The reaction solution was recorded as reaction solution \textcircled{1}.

In another 50 mL single-necked flask, M4 (4.1 g, 10.0 mmol), PyBOP (5.75 g, 11 mmol), HOBt (1.49 g, 11 mmol) and 10 mL DMF were added, and after dissolution, DIPEA (1.82 mL, 11 mmol) was added in an ice-water bath, and a reaction was continued for 40 min, then reaction solution \textcircled{1} was added, and the temperature was raised to room temperature to react for 2 h. The reaction progress was monitored by HPLC. After completion of the reaction, the reaction solution was purified by high performance liquid chromatography to obtain a preparation liquid. The preparation liquid was extracted with dichloromethane, washed with saturated sodium chloride solution, dried with anhydrous sodium sulfate, filtered, and concentrated to obtain 4.6 g of solid, at a yield of 68%; LC-MS: [M+H]⁺=676.7.

### Step 3: Compound 15d

Add 15b (2.0 g, 2.96 mmol) in a 25 mL single-necked flask, dissolve in 15 mL of DMF, then add 2.0 g of 5% Pd/C, and perform a hydrogenation reaction for 2 h. After the completion of the reaction, filtration was carried out, and the filtrate was placed in a bath of ice-water, and DIPEA (496 uL, 3.0 mmol) was added, followed by M3 (1.7 g, 2.96 mmol), and then the temperature was raised to room temperature to react for 1 h. When the reaction was complete as detected by HPLC, the reaction solution was purified by high performance liquid chromatography to obtain a preparation liquid, which was lyophilized to give 1120.0 mg of a product at 45% yield; LC-MS: [M−H]⁻=830.3.

### Step 4: Compound 15e

Add 15d (500 mg, 0.60 mmol), M5 (321 mg, 0.60 mmol), PyBOP (469 mg, 0.90 mmol), HOBt (121 mg, 0.90 mmol) and 15 mL of DMF into a 50 mL single-necked flask, add DIPEA (446 uL, 2.7 mmol) in an ice-water bath, raise to room temperature and react for 2 h. After the completion of the reaction as detected by HPLC, the reaction solution was purified by HPLC to obtain the preparation liquids of compound 15e-1 and compound 15e-2, and the preparation liquids were lyophilized to obtain 138 mg of compound 15e-1, LC-MS: [M+H]⁺=1249.5, and 140 mg of compound 15e-2, LC-MS: [M+H]⁺=1249.5, respectively.

### Step 5: Compound 15A

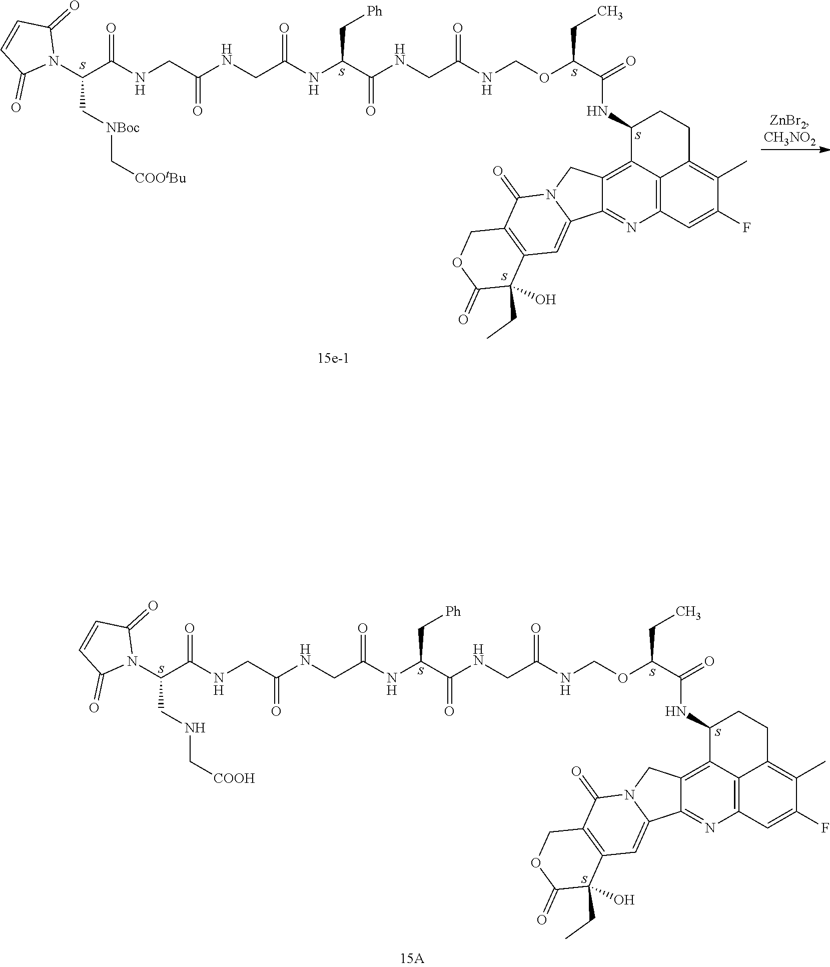

Add 15e-1 (100 mg, 0.08 mmol), zinc bromide (360 mg, 1.6 mmol) and 5 mL of nitromethane into a 25 mL single-necked flask and react at 40° C. for 1 h. After the completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain 59 mg of a solid; LC-MS: [M+H]⁺=1093.4.

### Step 6: Compound 15B

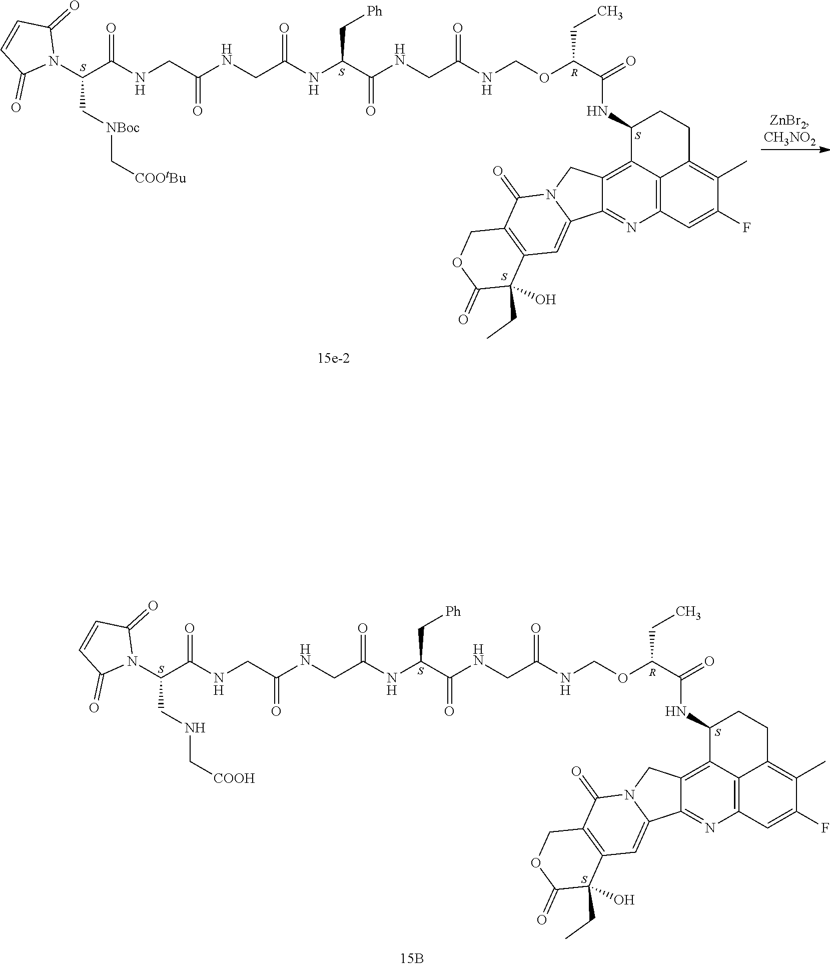

Add 15e-2 (100 mg, 0.08 mmol), zinc bromide (360 mg, 1.6 mmol) and 5 mL of nitromethane into a 25 mL single-necked flask and react at 40° C. for 1 h. After the completion of the reaction as detected by HPLC, the solvent was removed by concentration under reduced pressure, and a crude product was obtained. The crude product was purified by high performance liquid chromatography to obtain a product preparation liquid, and the preparation liquid was lyophilized to obtain 60 mg of solid; LC-MS: [M+H]⁺=1093.4.

### Example 27

### Synthesis of Compounds 16A and 16B

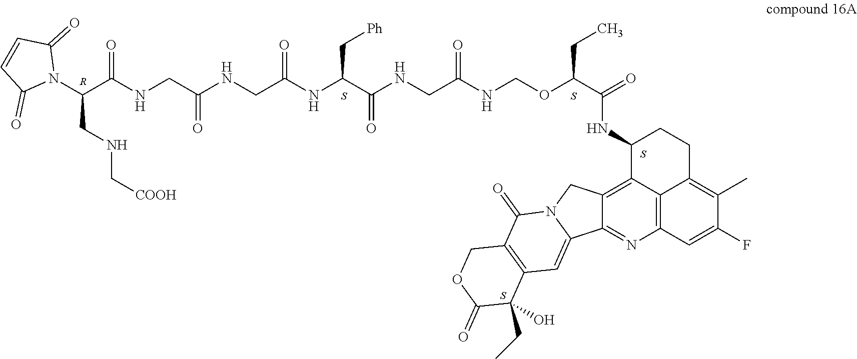

Referring to the synthetic route of Example 26, compound 16A (55 mg) was obtained; LC-MS: [M+H]⁺=1093.4.

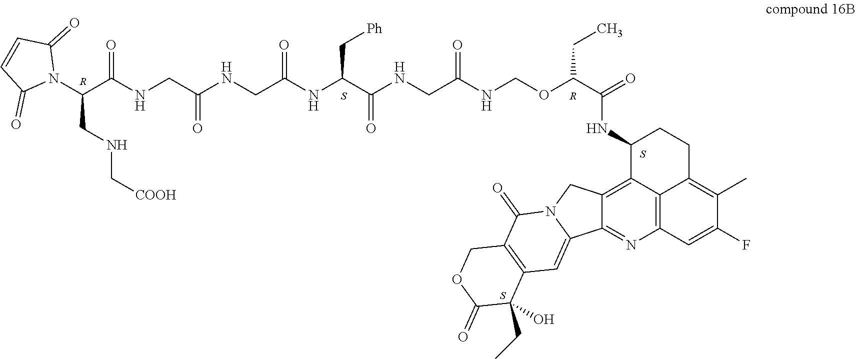

Referring to the synthetic route of Example 26, compound 16B (54 mg) was obtained; LC-MS: [M+H]⁺=1093.4.

### Example 28

### Synthesis of Compounds 17A and 17B

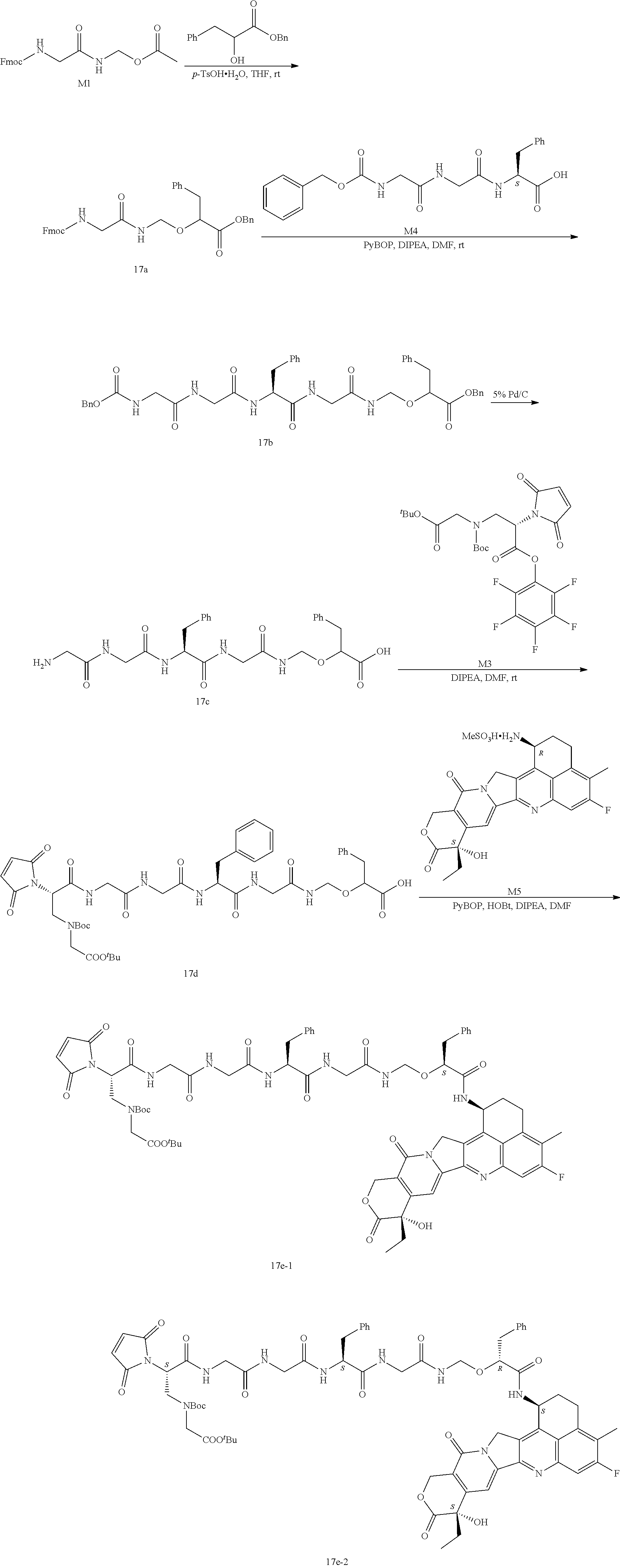

### Step 1: Compound 17a

Add M1 (10 g, 27.1 mmol), benzyl 2-hydroxy-phenylpropionate (synthesized by the method published in the literature Nature Communications, 2020. 11(1), 56.) (14.7 g, 54.3 mmol), zinc acetate (9.96 g, 54.3 mmol) and 100 mL of toluene into a 250 mL single-necked flask, heat to 100° C. and react for 4 h. After the reaction was completed, the temperature was lowered to room temperature, filtration was performed to remove insoluble material, and the filtrate was concentrated to obtain a crude product. The crude product was purified by silica gel column chromatography (PE:EA=10:1-5:1-2:1) to obtain 6.13 g of the target material at 40% yield; LC-MS: [M+H]⁺=565.6.

### Step 2: Compound 17b

In a 50 mL single-necked flask, add 17a (5 g, 8.86 mmol) and 15 mL of DMF, and after dissolution, add DBU (1.53 g, 10 mmol) in an ice-water bath and react for 1 h, and record the reaction solution as reaction solution \textcircled{1}.

In another 50 mL single-necked flask, add M4 (3.6 g, 8.86 mmol), PyBOP (5.23 g, 10 mmol), HOBt (1.36 g, 10 mmol) and 10 mL of DMF, and after dissolution, add DIPEA (1.65 mL, 10 mmol) in an ice-water bath, and continue reacting for 30 min, then add reaction solution \textcircled{1}, raise to room temperature and react for 2 h. The reaction progress was monitored by HPLC. After completion of the reaction, the reaction solution was purified by high performance liquid chromatography to obtain a preparation liquid. The preparation liquid was extracted with dichloromethane, washed with saturated sodium chloride solution, dried with anhydrous sodium sulfate, filtered, and concentrated to obtain 5.0 g of solid at 77% yield; LC-MS: [M+H]⁺=738.3.

### Step 3: Compound 17d

Add 17b (3.0 g, 4.07 mmol) in a 25 mL single-necked flask, and after dissolution with 15 mL of DMF, add 3.0 g of 5% Pd/C, and perform a hydrogenation reaction for 2 h. When the reaction was completed, filtration was performed, the filtrate was placed in an ice-water bath, and DIPEA (744 uL, 4.5 mmol) was added, followed by M3 (2.34 g, 4.07 mmol), and then the temperature was raised to room temperature to react for 1 h. When the reaction was complete as detected by HIPLC, the reaction solution was purified by HPLC to obtain a preparation liquid, which was lyophilized to give 1.2 g of a product at 33% yield; LC-MS: [M−H]⁻=892.4.

### Step 4: Compound 17e

17d (500 mg, 0.56 mmol), M5 (300 mg, 0.56 mmol), PyBOP (438 mg, 0.84 mmol), HOBt (113 mg, 0.84 mmol) and 15 mL of DMF were added to a 50 mL single-necked flask, DIPEA (330 uL, 2.0 mmol) was added in an ice-water bath, and the temperature was raised to room temperature to react for 2 h. After the reaction was complete as detected by HPLC, the reaction solution was purified by HPLC to obtain the preparation liquids of compound 17e-1 and compound 17e-2, and the preparation liquids were lyophilized to obtain 156 mg of compound 17e-1, LC-MS: [M+H]⁺=1311.4, and 150 mg of compound 17e-2, LC-MS: [M+H]⁺=1311.7, respectively.

### Step 5: Compound 17A

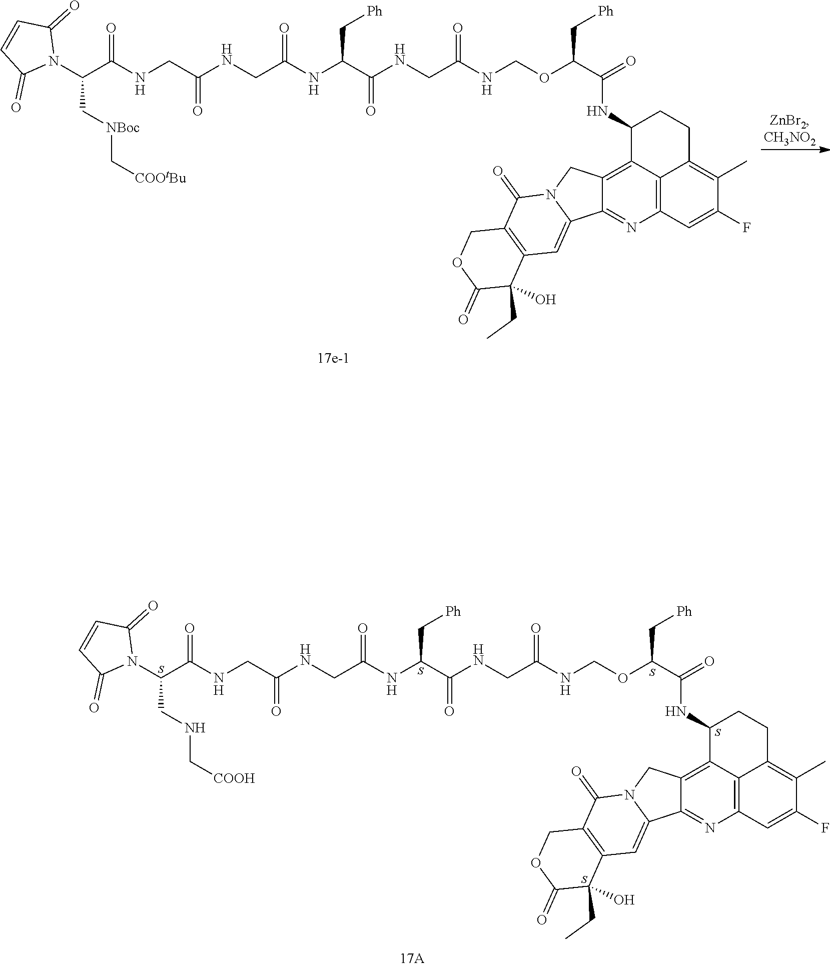

17e-1 (100 mg, 0.08 mmol), zinc bromide (360 mg, 1.6 mmol) and 5 mL of nitromethane were added to a 25 mL single-necked flask and a reaction was carried out at 40° C. for 1 h. After the reaction was complete as detected by HPLC, the solvent was removed by concentration under reduced pressure, and a crude product was obtained. The crude product was purified by high performance liquid chromatography to obtain a product preparation liquid, and the preparation liquid was lyophilized to obtain 43 mg of solid; LC-MS: [M+H]⁺=1155.4.

### Step 6: Compound 17B

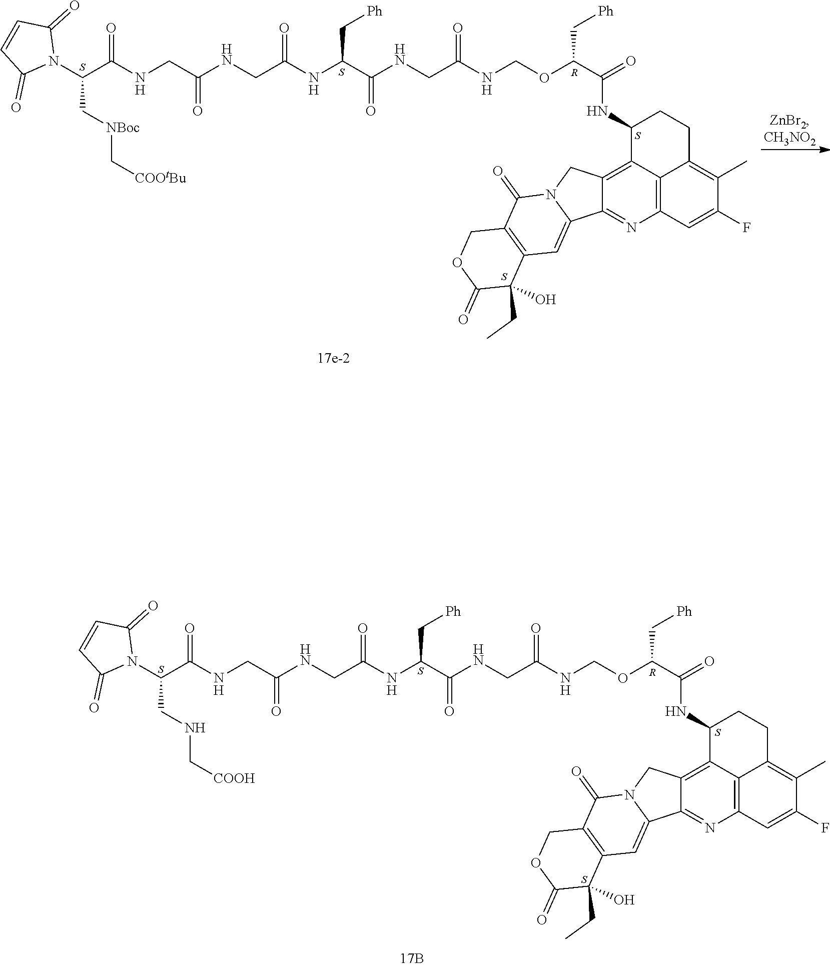

17e-2 (100 mg, 0.08 mmol), zinc bromide (360 mg, 1.6 mmol) and 5 mL of nitromethane were added to a 25 mL single-necked flask and a reaction was carried out at 40° C. for 1 h. After the reaction was complete as detected by HPLC, the solvent was removed by concentration under reduced pressure, and a crude product was obtained. The crude product was purified by high performance liquid chromatography to obtain a product preparation liquid, and the preparation liquid was lyophilized to obtain 40 mg of solid; LC-MS: [M+H]⁺=1155.4.

### Example 29

### Synthesis of Compounds 18A and 18B

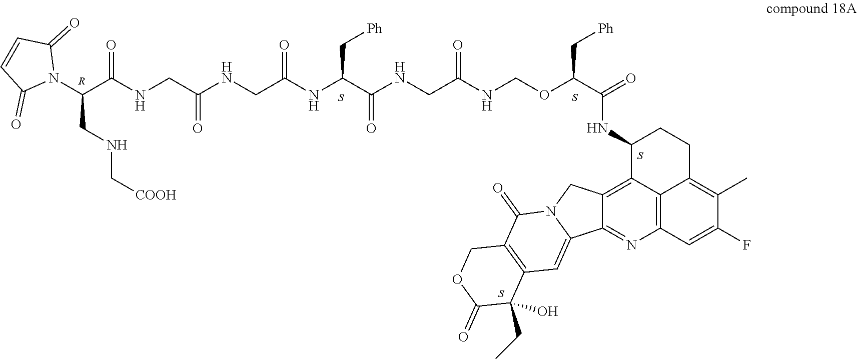

Referring to the synthetic route of Example 28, compound 18A (54 mg) was obtained; LC-MS: [M+H]⁺=1155.4.

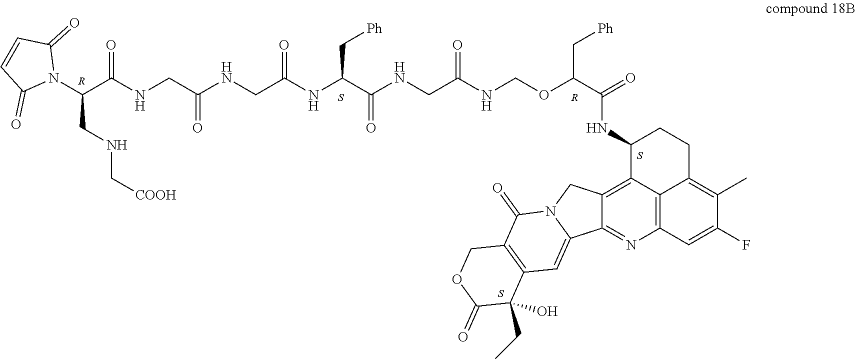

Referring to the synthetic route of Example 28, compound 18B (55 mg) was obtained; LC-MS: [M+H]⁺=1155.4.

### Example 30

### Synthesis of Compounds 19A and 19B

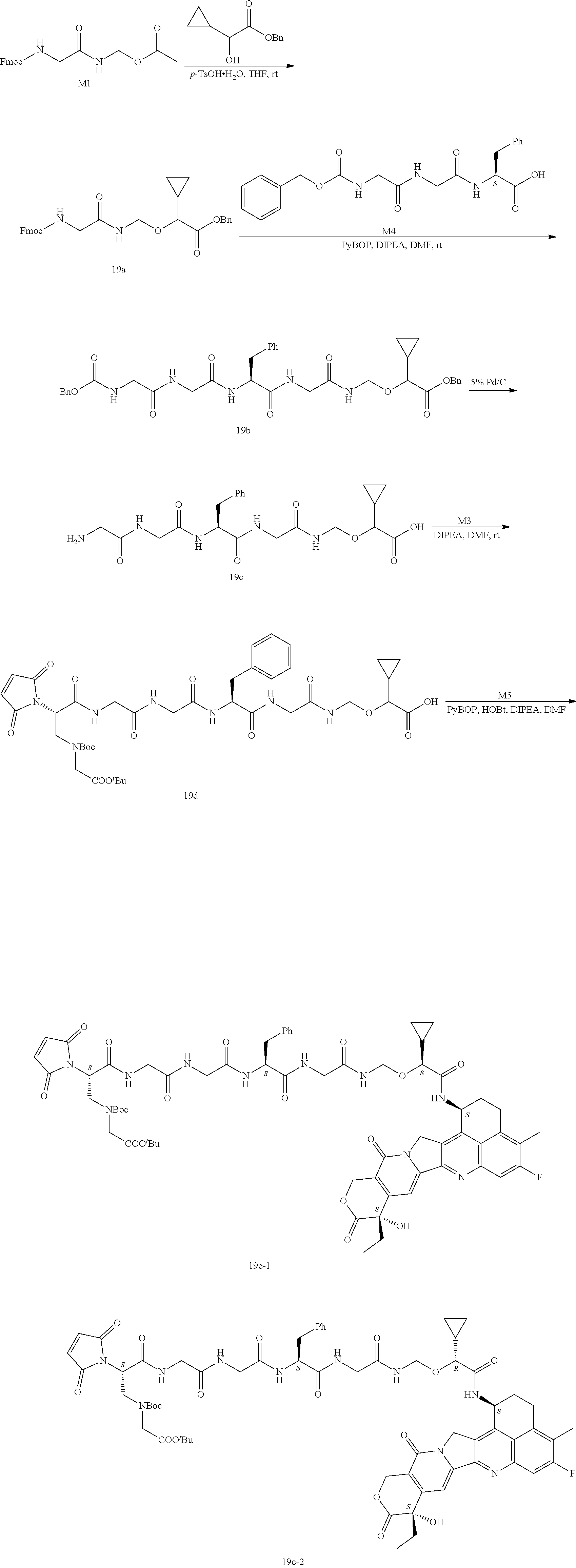

### Step 1: Compound 19a

Add M1 (10 g, 27.1 mmol), benzyl 2-cyclopropyl-2-hydroxyacetate (prepared according to the method published in patent WO2020244657A1) (11.2 g, 54.3 mmol), zinc acetate (9.96 g, 54.3 mmol) and 100 mL of toluene in a 250 mL single-necked flask, heat to 100° C. and react for 4 h. When the reaction was completed, the temperature was reduced to room temperature, filtering was performed to remove insoluble material, and the filtrate was concentrated to obtain a crude product. The crude product was purified by silica gel column chromatography (PE:EA=10:1-5:1-2:1) to obtain 4.97 g of the target material at 36% yield; LC-MS: [M+H]⁺=515.2.

### Step 2: Compound 19b

Add 19a (4 g, 7.8 mmol) and 10 mL of DMF in a 50 mL single-necked flask, and after dissolution, add DBU (1.42 g, 9.3 mmol) in an ice-water bath and react for 1 h. The reaction solution was recorded as reaction solution \textcircled{1}.

In another 50 mL single-necked flask, M4 (3.2 g, 7.8 mmol), PyBOP (4.5 g, 8.6 mmol), HOBt (1.16 g, 8.6 mmol) and 10 mL of DMF were added, and after dissolution, DIPEA (1.65 mL, 10 mmol) was added in an ice-water bath, and the reaction was continued for 30 min, then reaction solution \textcircled{1} was added, and the temperature was raised to room temperature to react for 2 h. The reaction process was monitored by HPLC, and after completion of the reaction, the reaction solution was purified by high performance liquid chromatography to obtain a preparation liquid. The preparation liquid was extracted with dichloromethane, washed with saturated sodium chloride solution, dried with anhydrous sodium sulfate, filtered, and concentrated to obtain 4.2 g of solid, with a yield of 78%; LC-MS: [M+H]⁺=688.3.

### Step 3: Compound 19d

In a 25 mL single-necked flask, 19b (1000 mg, 1.45 mmol) was added, and after dissolution with 15 mL of DMF, 1000 mg of 5% Pd/C was added, and a hydrogenation reaction was performed for 2 h. When the reaction was completed, filtering was performed, the filtrate was placed in an ice-water bath, and DIPEA (248 uL, 1.5 mmol) was added, followed by M3 (720 mg, 1.45 mmol), and then the temperature was raised to room temperature to react for 1 h. When the reaction was complete as detected by HPLC, the reaction solution was purified by HPLC to obtain a preparation liquid, and the preparation liquid was lyophilized to obtain 503 mg of product at 41% yield; LC-MS: [M−H]⁻=842.3.

### Step 4: Compounds 19e-1 and 19e-2

19d (500 mg, 0.59 mmol), M5 (317 mg, 0.59 mmol), PyBOP (339 mg, 0.65 mmol), HOBt (88 mg, 0.86 mmol) and 10 mL of DMF were added to a 50 mL single-necked flask, and DIPEA (292 uL, 1.77 mmol) was added in an ice-water bath, and the temperature was raised to room temperature to react for 2 h. After the reaction was complete as detected by HPLC, the reaction solution was purified by HPLC to obtain the preparation liquids of compound 19e-1 and compound 19e-2, and the preparation liquids were lyophilized to obtain 112 mg of compound 19e-1, LC-MS: [M+H]⁺=1261.5, and 131 mg of compound 19e-2, LC-MS: [M+H]⁺=1261.5, respectively.

### Step 5: Compound 19A

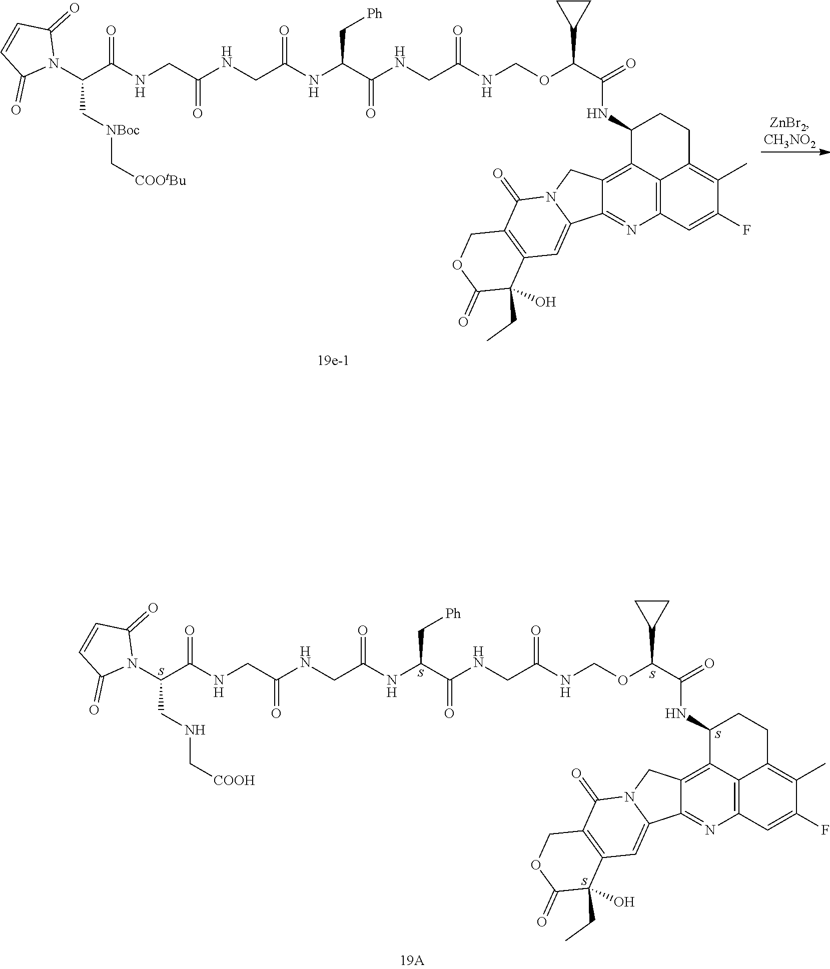

19e-1 (100 mg, 0.079 mmol), zinc bromide (357 mg, 1.59 mmol) and 5 mL of nitromethane were added to a 25 mL single-necked flask and a reaction was carried out at 40° C. for 1 h. After the completion of the reaction as detected by HPLC, the solvent was removed by concentration under reduced pressure, to obtain a crude product. The crude product was purified by high performance liquid chromatography to obtain a product preparation liquid, and the preparation liquid was lyophilized to obtain 55 mg of solid; LC-MS: [M+H]⁺=1105.4.

### Step 6: Compound 19B

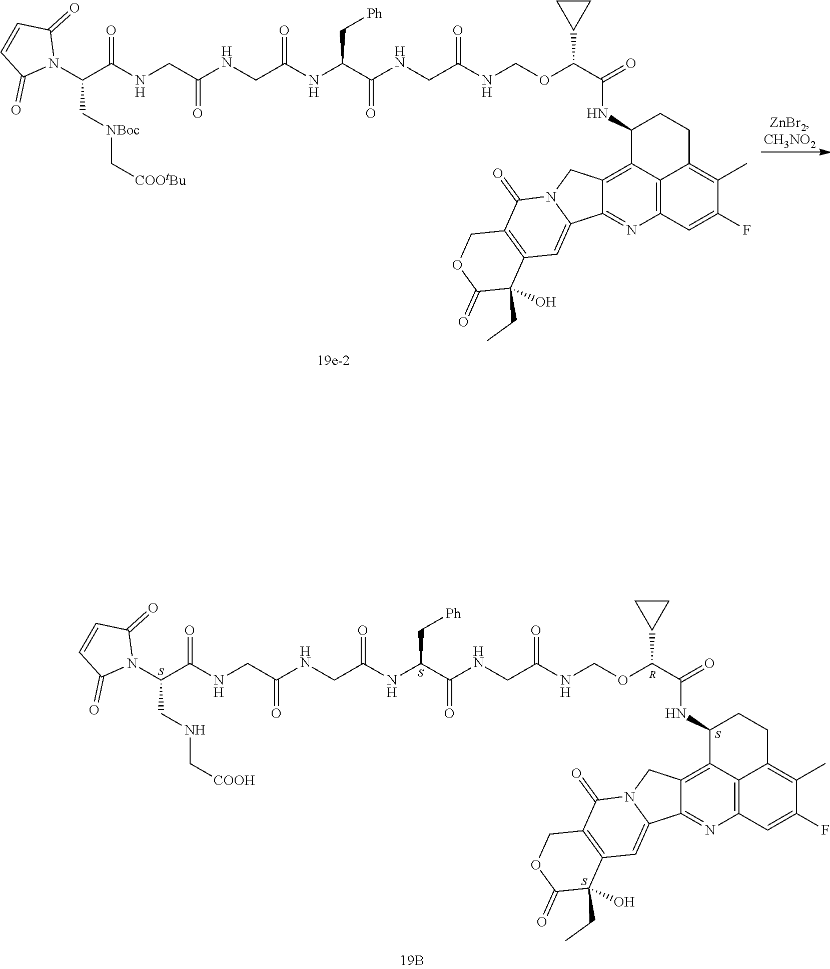

19e-2 (100 mg, 0.079 mmol), zinc bromide (357 mg, 1.59 mmol) and 5 mL of nitromethane were added to a 25 mL single-necked flask and a reaction was carried out at 40° C. for 1 h. After the reaction was completed as detected by HPLC, the solvent was removed by concentration under reduced pressure, and a crude product was obtained. The crude product was purified by high performance liquid chromatography to obtain a product preparation liquid, and the preparation liquid was lyophilized to obtain 58 mg of a solid; LC-MS: [M+H]⁺=1105.4.

### Example 31

### Synthesis of Compounds 20A and 20B

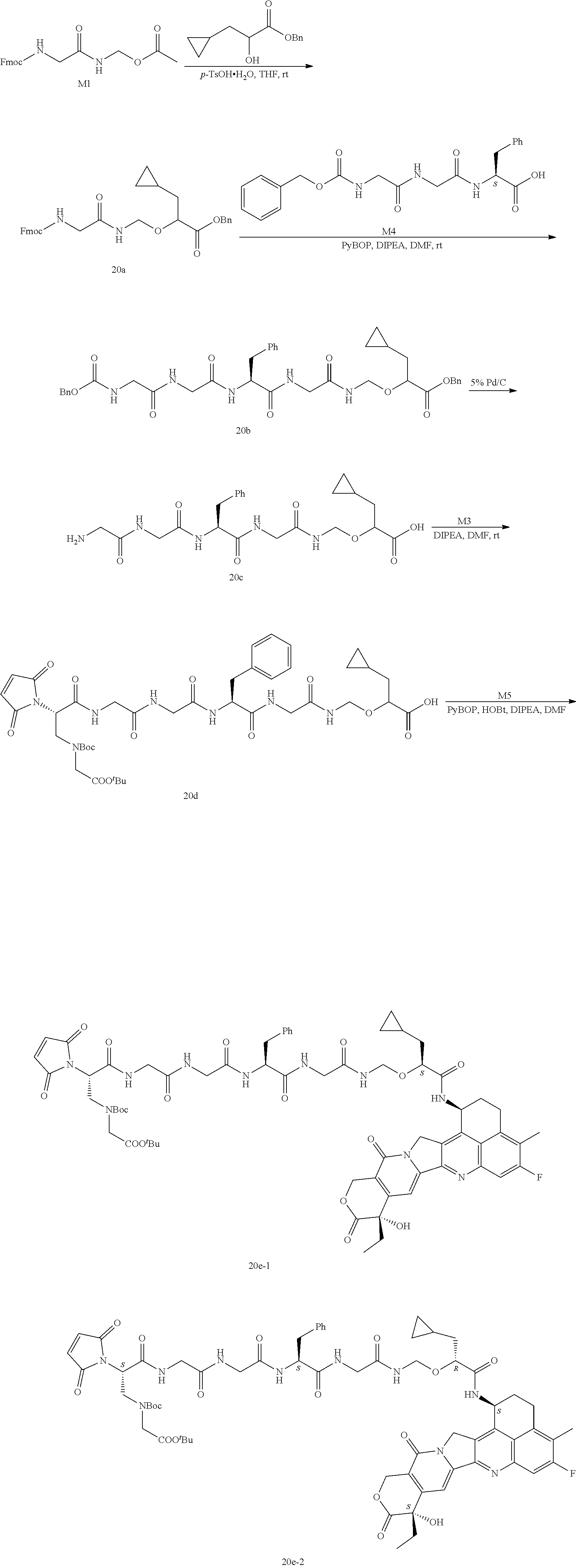

### Step 1: Compound 20a

Add M1 (10 g, 27.1 mmol), benzyl 2-hydroxy-cyclopropylpropionate (synthesized by the method published in patent WO2020063676A) (12.0 g, 54.3 mmol), zinc acetate (9.96 g, 54.3 mmol) and 100 mL of toluene in a 250 mL single-necked flask, heat to 100° C. and react for 4 h. When the reaction is completed, reduce to room temperature. Filter to remove insoluble material, and the filtrate is concentrated to obtain a crude product. The crude product was purified by silica gel column chromatography (PE:EA=10:1-5:1-2:1) to obtain 5.09 g of the target material; LC-MS: [M+H]⁺=529.2.

### Step 2: Compound 20b

20a (4 g, 7.6 mmol) and 10 mL of DMF were added to a 50 mL single-necked flask, and after dissolution, DBU (1.39 g, 9.1 mmol) was added in an ice-water bath and a reaction was carried out for 1 h, and the reaction solution was recorded as reaction solution \textcircled{1}.

In another 50 mL single-necked flask, M4 (3.12 g, 7.6 mmol), PyBOP (4.5 g, 8.6 mmol), HOBt (1.16 g, 8.6 mmol) and 10 mL of DMF were added, and after dissolution, DIPEA (1.65 mL, 10 mmol) was added in an ice-water bath and a reaction was continued for 30 min, and then the reaction solution \textcircled{1} was added, and the temperature was raised to room temperature to react for 2 h. HPLC was performed to monitor the reaction progress, and when the reaction was complete, the reaction solution was purified by high performance liquid chromatography to obtain a preparation liquid. The preparation liquid was extracted with dichloromethane, washed with saturated sodium chloride solution, dried with anhydrous sodium sulfate, filtered, and concentrated to obtain 4.5 g of solid, with a yield of 84%; LC-MS: [M+H]⁺=702.3.

### Step 3: Compound 20d

Add 20b (1000 mg, 1.42 mmol) in a 25 mL single-necked flask, and after dissolution with 15 mL of DMF, add 1000 mg of 5% Pd/C, and perform a hydrogenation reaction for 2 h. After the reaction was completed, filtration was performed, the filtrate was placed in an ice-water bath, and then DIPEA (248 uL, 1.5 mmol) was added, followed by M5 (708 mg, 1.42 mmol), and the temperature was raised to room temperature to react for 1 h. When the reaction was complete as detected by HPLC, the reaction solution was purified by HPLC to obtain a preparation liquid, which was lyophilized to obtain 443 mg of product at 36% yield; LC-MS: [M−H]⁻=856.4.

### Step 4: Compounds 20e-1 and 20e-2

20d (400 mg, 0.47 mmol), exatecan mesylate (250 mg, 0.47 mmol), PyBOP (223 mg, 0.56 mmol), HOBt (83 mg, 0.56 mmol) and 10 mL of DMF were added to a 50 mL single-necked flask, DIPEA (248 uL, 1.5 mmol) was added in an ice-water bath, and the temperature was raised to room temperature to react for 2 h. After the reaction was complete as detected by HPLC, the reaction solution was purified by HPLC to obtain the preparation liquids of compound 20e-1 and compound 20e-2, which were lyophilized to obtain 103 mg of compound 20e-1, LC-MS: [M+H]⁺=1275.5, and 103 mg of compound 20e-2, LC-MS: [M+H]⁺=1275.5, respectively.

### Step 5: Compound 20A

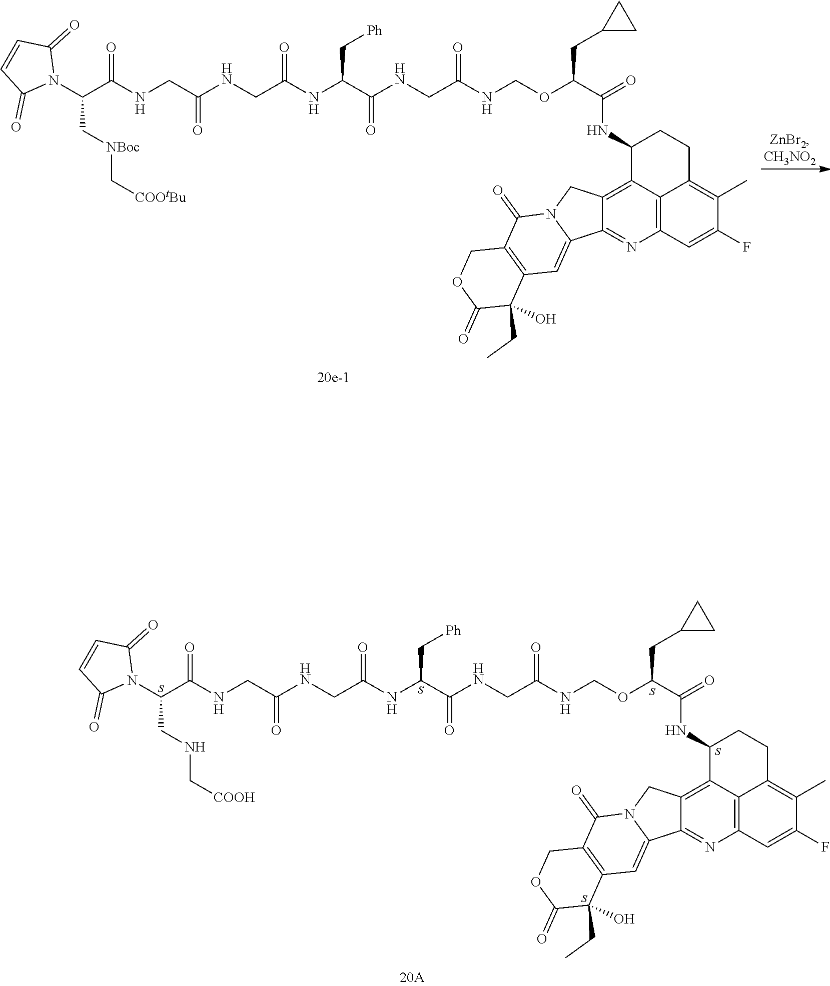

In a 25 mL single-necked flask, 8A (100 mg, 0.078 mmol), zinc bromide (352 mg, 1.57 mmol) and 5 mL of nitromethane were added, and a reaction was carried out at 40° C. for 1 h. After the completion of the reaction as detected by HPLC, the solvent was removed by concentration under reduced pressure and a crude product was obtained. The crude product was purified by high performance liquid chromatography to obtain a product preparation liquid, and the preparation liquid was lyophilized to obtain 51 mg of solid; LC-MS: [M+H]⁺=1119.4.

### Step 6: Compound 20B

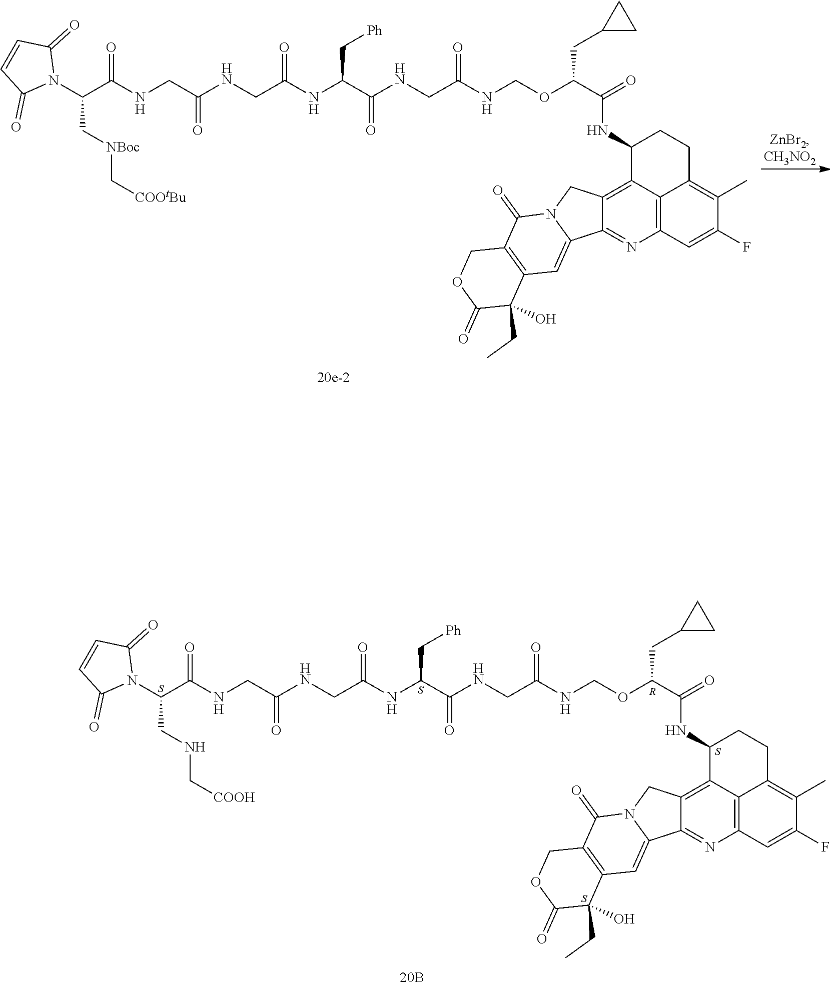

20e-2 (100 mg, 0.079 mmol), zinc bromide (357 mg, 1.59 mmol) and 5 mL of nitromethane were added to a 25 mL single-necked flask and a reaction was carried out at 40° C. for 1 h. After the reaction was complete as detected by HPLC, the solvent was removed by concentration under reduced pressure, and a crude product was obtained. The crude product was purified by high performance liquid chromatography to obtain a product preparation liquid, and the preparation liquid was lyophilized to obtain 47 mg of a solid; LC-MS: [M+H]⁺=1119.4.

### Example 32

### Synthesis of Compound 21

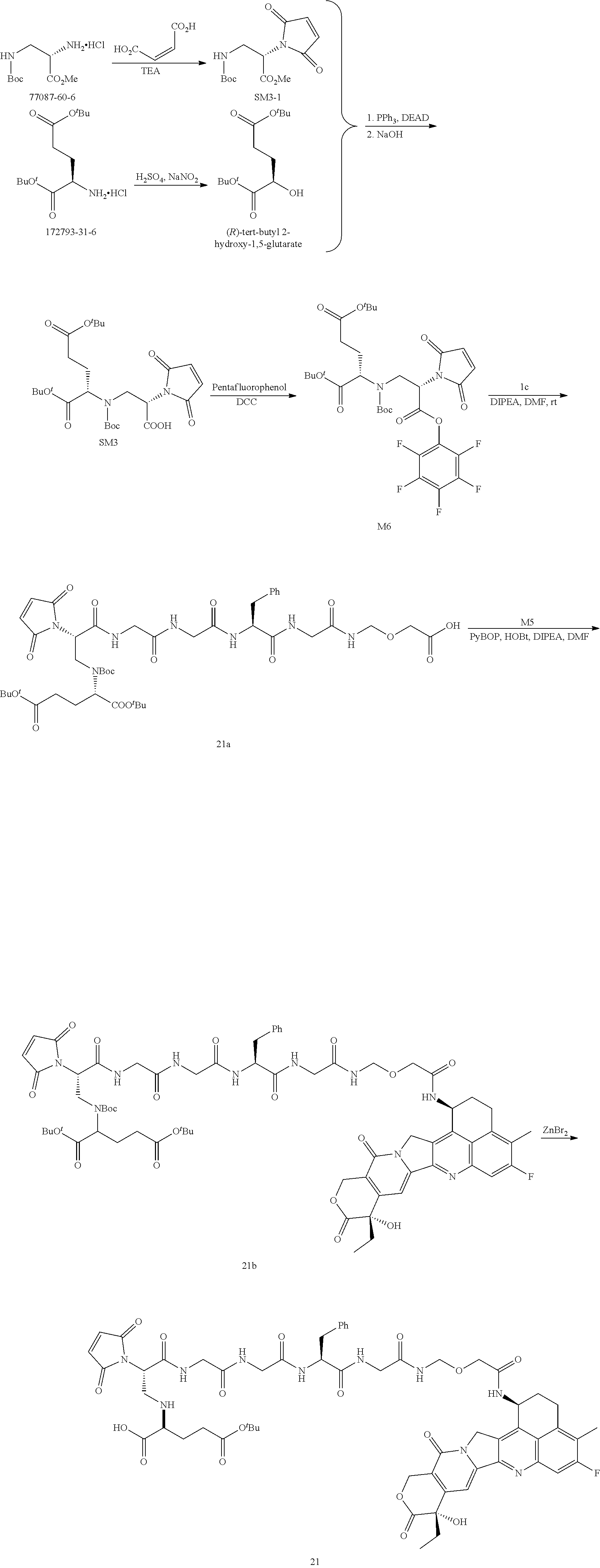

### Step 1: Compound SM3-1

Add 77087-60-6 (100 g, 458 mmol), maleic acid (53.4 g, 460 mmol), TEA (64 mL, 460 mmol) and 1000 mL toluene in a 2000 mL single-necked flask and heat to 100° C. to react for 5 h. After the reaction was completed, the temperature was lowered to room temperature, and then filtering was performed to remove insoluble material, and the filtrate was concentrated to obtain a crude product. The crude product was purified by silica gel column chromatography (PE:EA=100:1-50:1-20:1) to obtain 75.6 g of the target material; LC-MS: [M+H]⁺=299.1.

### Step 2: Compound (R)-tert-butyl 2-hydroxy-1,5-glutarate

Add 172793-31-6 (100 g, 338 mmol) and 1000 mL water in a 2000 mL single-necked flask, then sequentially add sodium nitrite (35 g, 507 mmol) and concentrated sulfuric acid (32 mL, 35 mmol), and slowly raise the temperature to room temperature to react for 24 h. After the reaction was completed, extraction was performed with 500 mL of ethyl acetate three times, and the organic phase was dried with anhydrous sodium sulfate, filtered, and concentrated under reduced pressure to remove the solvent, and a crude product was obtained. The crude product was purified by silica gel column chromatography (PE:EA=50:1-30:1-2:1) to obtain 91.2 g of the target material; LC-MS: [M+H]⁺=261.4.

### Step 3: Compound SM3

Add (R)-tert-butyl 2-hydroxy-1,5-glutarate (50 g, 192 mmol) and 1000 mL of anhydrous tetrahydrofuran into a 2000 mL single-necked flask, cool down the temperature to 0° C. in an ice-water bath, then add PPh₃ (87.7 g, 288 mmol), DEAD (50.2 g, 288 mmol) and SM3-1 (57.3, 192 mmol) in sequence. The temperature was slowly raised to room temperature to react for 13 h. After completion of the reaction, the insoluble material was removed by filtration and the filtrate was concentrated to obtain a crude product. The crude product was purified by silica gel column chromatography (PE:EA=50:1-30:1-1:1) to obtain 68.6 g of product.

The above product was dissolved in 500 mL of methanol, cooled to 0° C. in an ice-water bath, NaOH (64 mL, 190 mmol, 3M/L) was added dropwise at this temperature, and a reaction was performed for 12 h while maintaining this temperature. Next, the pH was adjusted to 3 by the addition of HCl (6 M/L), extraction was performed five times with 500 mL of dichloromethane, drying was performed with anhydrous sodium sulfate, followed by filtering, the filtrate was concentrated under reduced pressure, and the crude product obtained was purified by column chromatography (DCM/MeOH=50/1-20/1-2/1), to obtain 50.4 g of SM3; LC-MS: [M−H]⁻=525.5.

### Step 4: Compound M6

In a 2000 mL single-necked flask, compound SM3 (50 g, 95 mmol, 1.0 eq), pentafluorophenol (19.2 g, 104.5 mmol, 1.1 eq), DCC (21.5 g, 104.5 mmol, 1.1 eq) and THF (600 mL) were added, and a reaction was carried out at room temperature for 1 h (monitored by TLC), and insoluble material was filtered off. The reaction solution was purified directly by preparative LC, and the preparation liquid was concentrated at 35° C. in a water bath under reduced pressure by water pump to remove acetonitrile, and lyophilized to obtain compound M6 (51.9 g) at 79% yield; LC-MS: [M+H]⁺=693.3.

### Step 5: Compound 21a

In a 25 mL single-necked flask, 1c (1 g, 2.36 mmol) was added, and after dissolution with 25 mL DMF, DIPEA (430 uL, 2.6 mmol) was added, then M6 (1177 mg, 2.36 mmol) was added, and then the temperature was raised to room temperature to react for 1 h. The completion of the reaction was detected by HPLC, and the reaction solution was purified by high performance liquid chromatography to obtain a preparation liquid, which was lyophilized to yield 555 mg of product; LC-MS: [M−H]⁻=931.0.

### Step 6: Compound 21b

In a 100 mL single-necked flask, 21a (500 mg, 0.54 mmol), exatecan mesylate M5 (285 mg, 0.54 mmol), PyBOP (239 mg, 0.6 mmol), HOBt (239 mg, 0.6 mmol) and 10 mL of DMF were added, DIPEA (248 uL, 1.5 mmol) was added in an ice-water bath, and the temperature was raised to room temperature to react for 2 h. After the completion of the reaction as detected by HPLC, the reaction solution was purified by HPLC to obtain the preparation liquid of compound 21b, which was lyophilized to give 231 mg of the compound; LC-MS: [M+H]⁺=1349.5.

### Step 7: Compound 21

Compound 21b (200 mg, 0.1488 mmol), zinc bromide (665 mg, 2.96 mmol) and 10 mL of nitromethane were added to a 25 mL single-necked flask and a reaction was carried out at 40° C. for 1 h. After the reaction was complete as detected by HPLC, the solvent was removed by concentration under reduced pressure, and a crude product was obtained. The crude product was purified by high performance liquid chromatography to obtain the product preparation liquid, and the preparation liquid was lyophilized to obtain 103 mg of solid; LC-MS: [M+H]⁺=1137.5.

### Example 33

### Synthesis of Compound 22

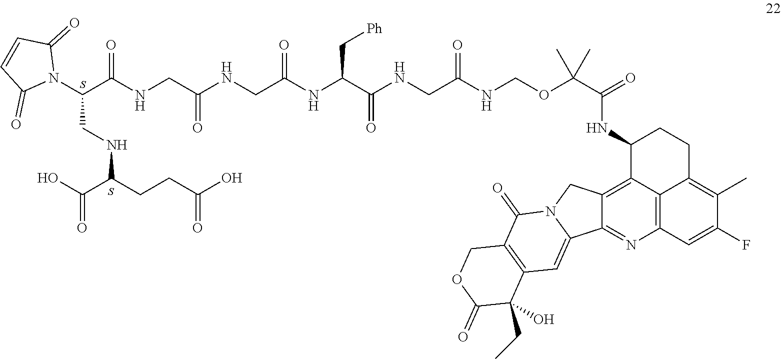

Using compounds M6 and 3c as starting materials and referring to the synthetic route of Example 32, compound 22 (91 mg) was obtained; LC-MS: [M+H]⁺=1165.5.

### Example 34

### Synthesis of Compounds 23 and 24

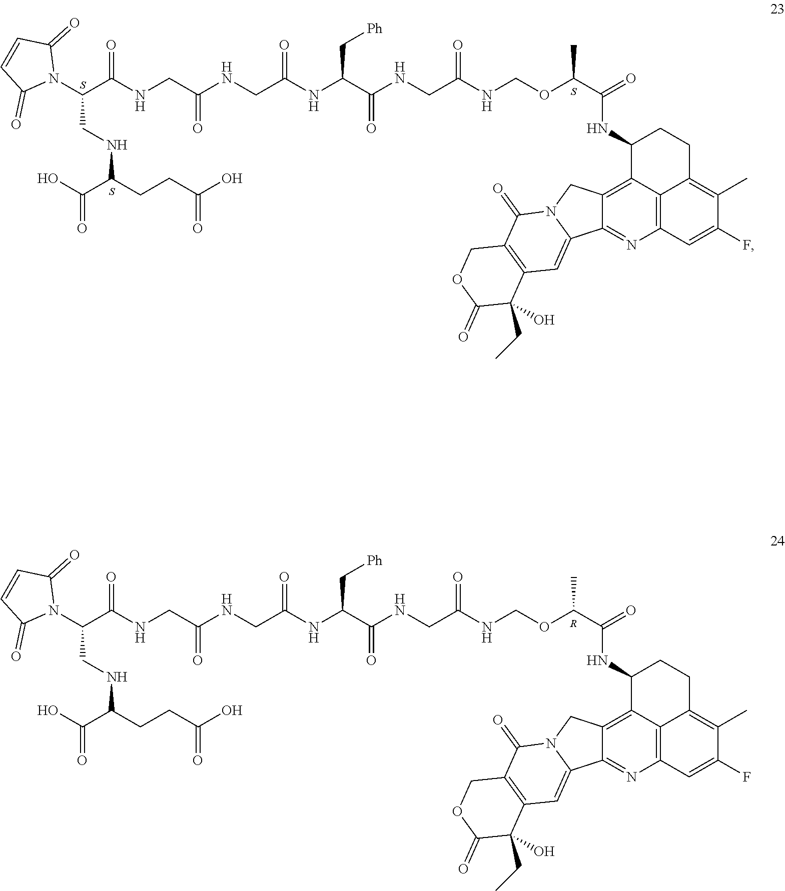

Using compounds M6 and 5c as starting materials and referring to the synthetic route of Example 32, 102 mg of compound 23 was obtained, LC-MS: [M+H]⁺=1151.4; 99 mg of compound 24 was obtained, LC-MS: [M+H]⁺=1151.4.

### Example 35

### Synthesis of Compounds 25 and 26

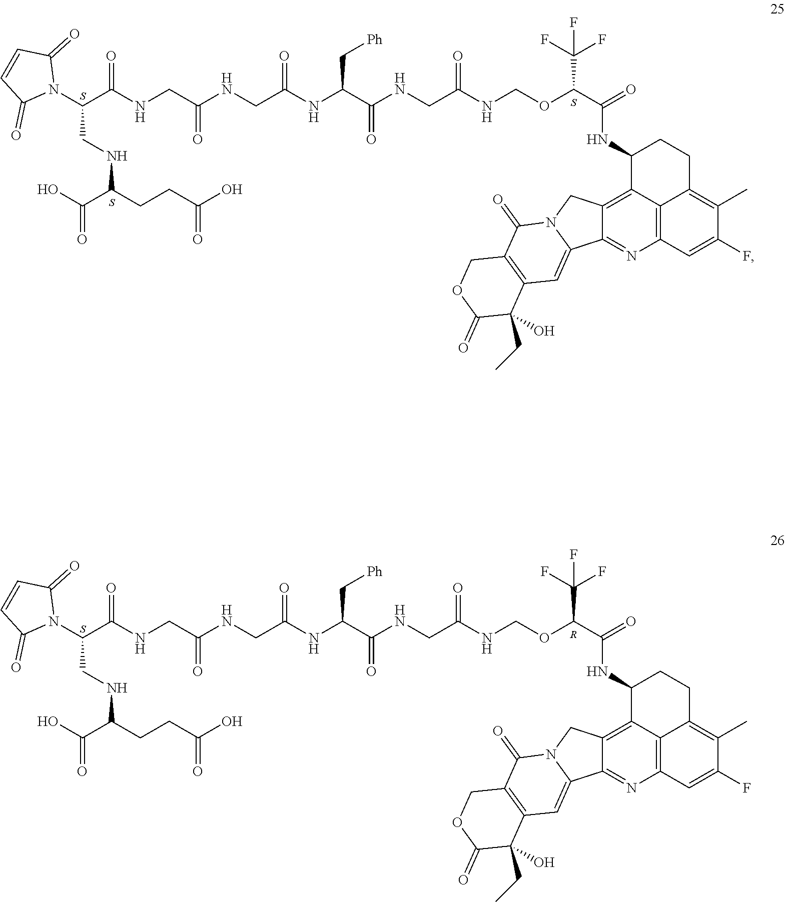

Using compounds M6 and 7c as starting materials and referring to the synthetic route of Example 32, 83 mg of compound 25 was obtained, LC-MS: [M+H]⁺=1205.7; 80 mg of compound 26 was obtained, LC-MS: [M+H]⁺=1205.7.

### Example 36

### Synthesis of Compounds 27 and 28

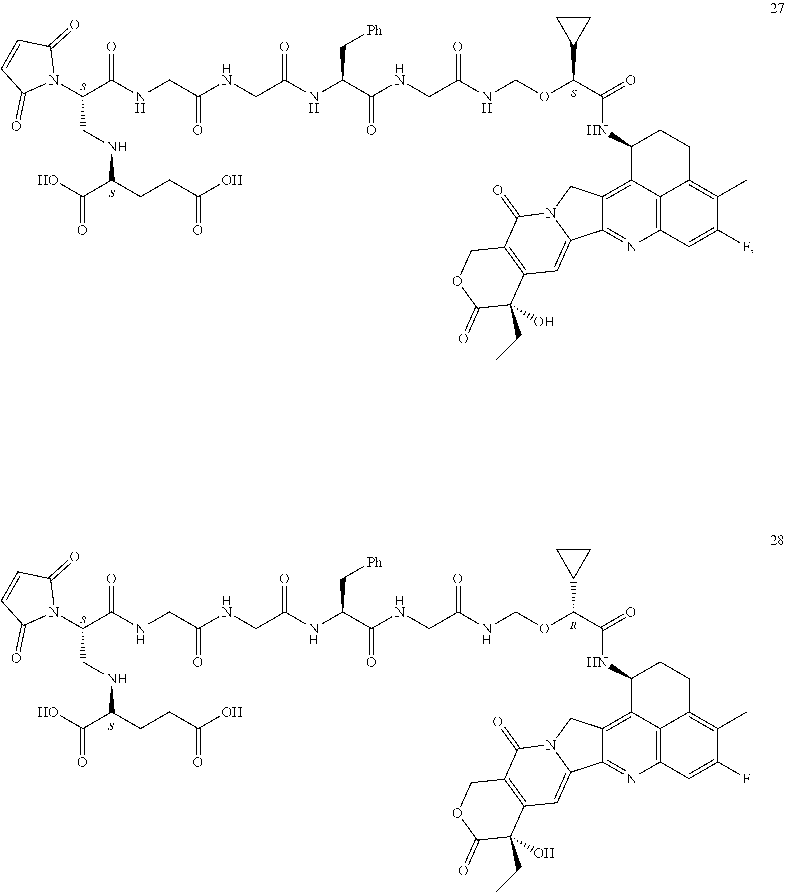

Using compounds M6 and 19c as starting materials and referring to the synthetic route of Example 32, 100 mg of compound 27 was obtained, LC-MS: [M+H]⁺=1177.5; 101 mg of compound 28 was obtained, LC-MS: [M+H]⁺=1177.5.

### Example 37

### Synthesis of Compound 29

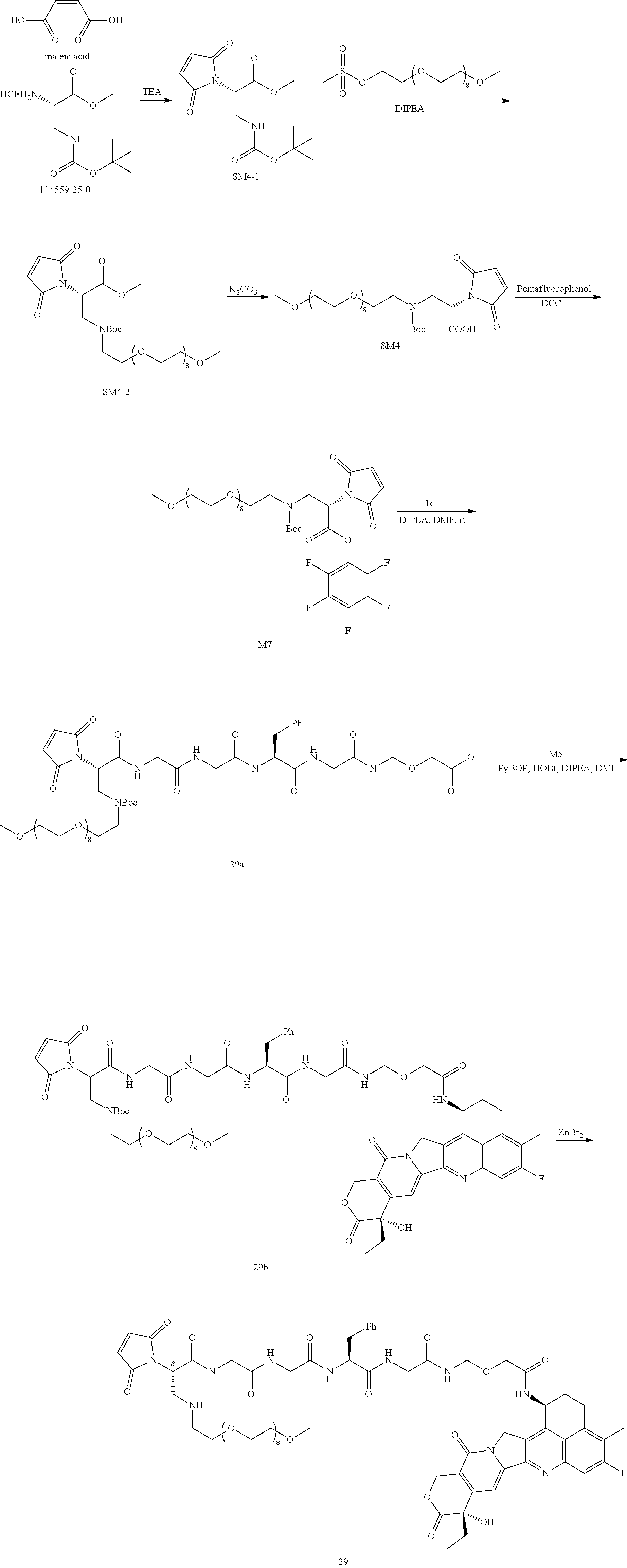

### Step 1: Compound SM4-1

In a 5000 mL single-necked flask, maleic acid (50 g, 431 mmol, 1.0 eq), 114559-25-0 (110 g, 431 mmol, 1 eq), TEA (263 g, 2.16 mol, 5 eq) and toluene (2000 mL) were added, heating was performed to react under reflux for 5 h (monitored by TLC), and insoluble material was filtered off. The reaction solution was directly subjected to rotary distillation under reduced pressure to remove solvent, and the residue was subjected to silica gel column chromatography (PE/EA=50/1-20/1-1/1) to obtain SM4-1 (64.7 g) at 50% yield; LC-MS: [M+H]⁺=299.2.

### Step 2: Compound SM4-2

SM4-1 (64 g, 215 mmol) was added into a 2000 mL single-necked flask, and after dissolution with 1000 mL of DMF, DIPEA (71 mL, 430 mmol) was added, and then nonaethylene glycol monomethyl ether methanesulfonate (111.5 g, 220 mmol) was added, and then the temperature was raised to room temperature to react for 2 h. The completion of the reaction was detected by HPLC, and the reaction solution was purified by silica gel column chromatography (PE/EA=50/1-20/1-1/1) to obtain 59.9 g of product; LC-MS: [M+H]⁺=709.4.

### Step 3: Compound SM4

SM4-2 (59 g, 83 mmol) was added into a 2000 mL single-necked flask, and after dissolution with 1000 mL MeOH, K₂CO₃ (11.75 g, 85 mmol) was added, and then a reaction was carried out at room temperature for 4 h. When the reaction was complete as detected by HPLC, insoluble materials were removed by filtration, and the reaction solution was purified directly by preparative LC, and the preparation liquid was concentrated at 35° C. in a water bath under reduced pressure by water pump to remove acetonitrile, and then lyophilized to obtain compound SM4 (27 g); LC-MS: [M−H]=693.5.

### Step 4: Compound M7

In a 500 mL single-necked flask, compound SM4 (25 g, 36 mmol, 1.0 eq), pentafluorophenol (7.3 g, 40 mmol, 1.1 eq), DCC (8.2 g, 40 mmol, 1.1 eq) and THF (200 mL) were added, a reaction was carried out at room temperature for 1 h (monitored by TLC), and insoluble material was filtered off. The reaction solution was purified directly by preparative LC, and the preparation liquid was concentrated at 35° C. in a water bath under reduced pressure by water pump to remove acetonitrile, and lyophilized to obtain compound M7 (23.3 g) at 93% yield; LC-MS: [M+H]⁺=695.8.

### Step 5: Compound 29a

In a 25 mL single-necked flask, 1c (1 g, 2.36 mmol) was added, and after dissolution with 25 mL DMF, DIPEA (430 uL, 2.6 mmol) was added, then M7 (1640 mg, 2.36 mmol) was added, and then the temperature was raised to room temperature to react for 1 h. The completion of the reaction was detected by HPLC, and the reaction solution was purified by high performance liquid chromatography to obtain a preparation liquid, which was lyophilized to obtain 609 mg of product; LC-MS: [M−H]⁻=1098.5.

### Step 6: Compound 29b

29a (500 mg, 0.45 mmol), exatecan mesylate M5 (240 mg, 0.45 mmol), PyBOP (215 mg, 0.54 mmol), HOBt (215 mg, 0.54 mmol) and 10 mL of DMF were added to a 100 mL single-necked flask, DIPEA (248 uL, 1.5 mmol) was added in an ice-water bath, and the temperature was raised to room temperature to react for 2 h. After the completion of the reaction as detected by HPLC, the reaction solution was purified by HPLC to obtain the preparation liquid of compound 29b, and the preparation liquid was lyophilized to obtain 187 mg of the compound; LC-MS: [M+H]⁺=1517.6.

### Step 7: Compound 29

Compound 29b (150 mg, 0.988 mmol), zinc bromide (223 mg, 0.988 mmol) and 10 mL of nitromethane were added to a 25 mL single-necked flask and a reaction was carried out at 40° C. for 1 h. After the reaction was complete as detected by HPLC, the solvent was removed by concentration under reduced pressure, and a crude product was obtained. The crude product was purified by high performance liquid chromatography to obtain the product preparation liquid, and the preparation liquid was lyophilized to obtain 114 mg of solid; LC-MS: [M+H]⁺=1517.9.

### Example 38

### Synthesis of Compound 30

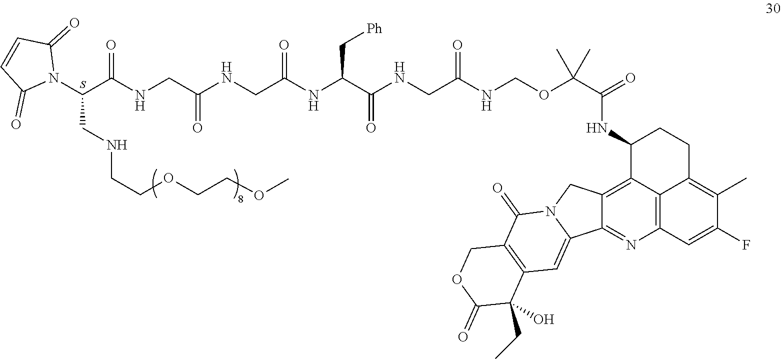

Using compounds M7 and 3c as starting materials and referring to the synthetic route of Example 37, compound 30 (125 mg) was obtained; LC-MS: [M+H]⁺=1445.6.

### Example 39

### Synthesis of Compounds 31 and 32

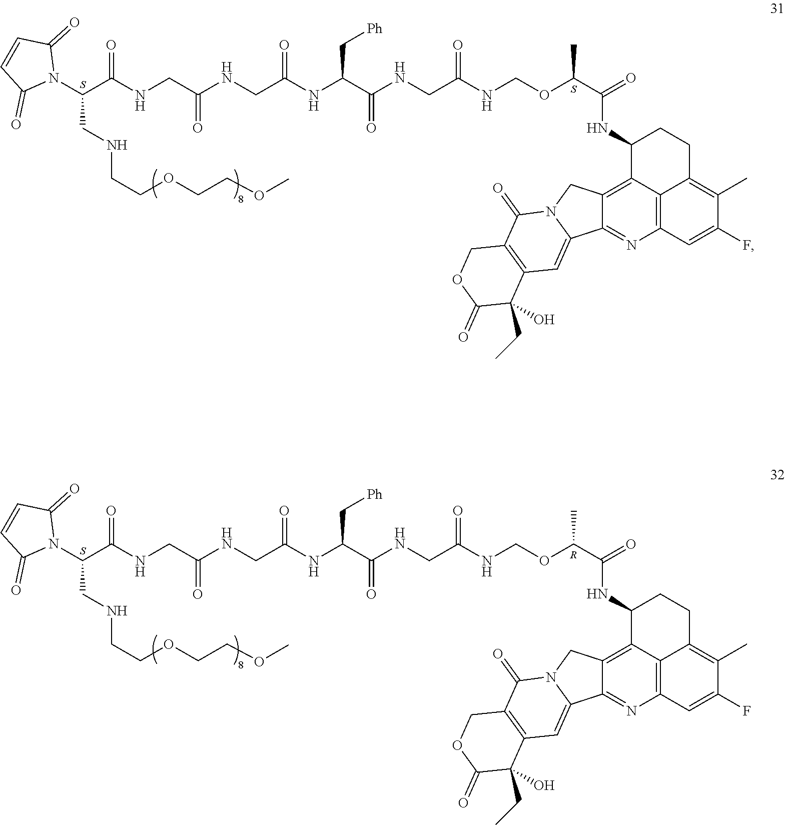

Using compounds M7 and 5c as starting materials and referring to the synthetic route of Example 37, 61 mg of compound 31 was obtained, LC-MS: [M+H]⁺=1431.7; 63 mg of compound 32 was obtained, LC-MS: [M+H]⁺=1431.7.

### Example 40

### Synthesis of Compounds 33 and 34

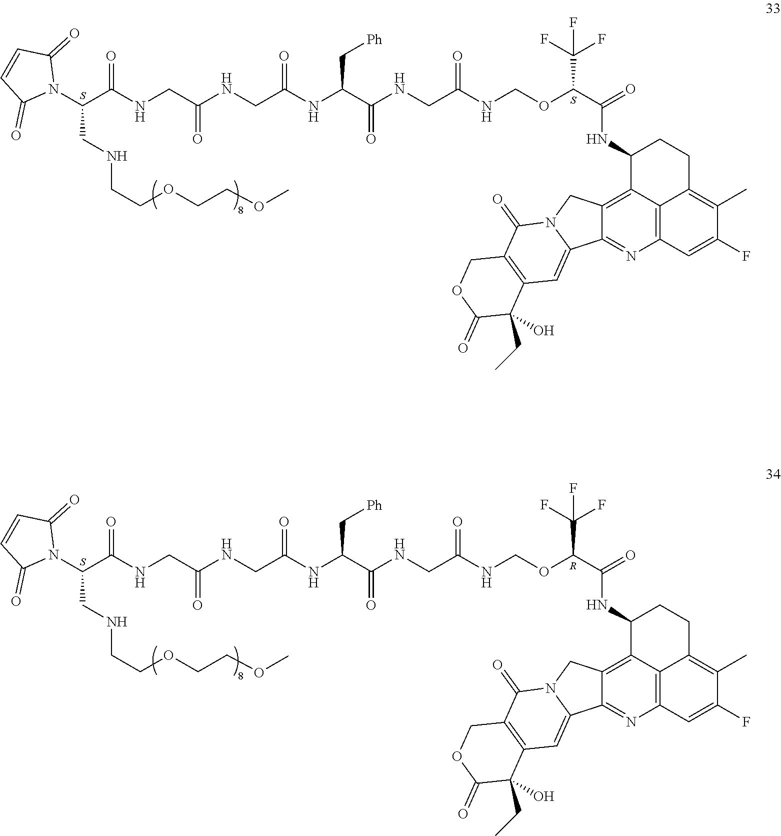

Using compounds M7 and 7c as starting materials, referring to the synthetic route of Example 37, 60 mg of compound 33 was obtained, LC-MS: [M+H]⁺=1485.6; 58 mg of compound 34 was obtained, LC-MS: [M+H]⁺=1485.6.

### Example 41

### Synthesis of Compounds 35 and 36

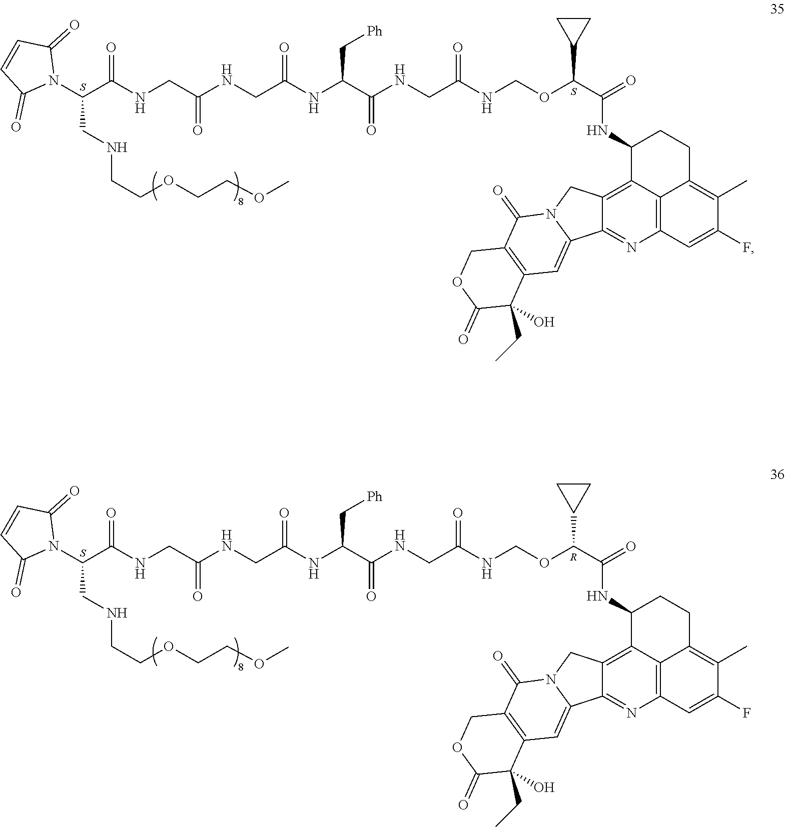

Using compounds M7 and 19c as starting materials and referring to the synthetic route of Example 37, 102 mg of compound 35 was obtained, LC-MS: [M+H]⁺=1457.8; 102 mg of compound 36 was obtained, LC-MS: [M+H]⁺=1457.8.

### Example 42

### Synthesis of Compound 37

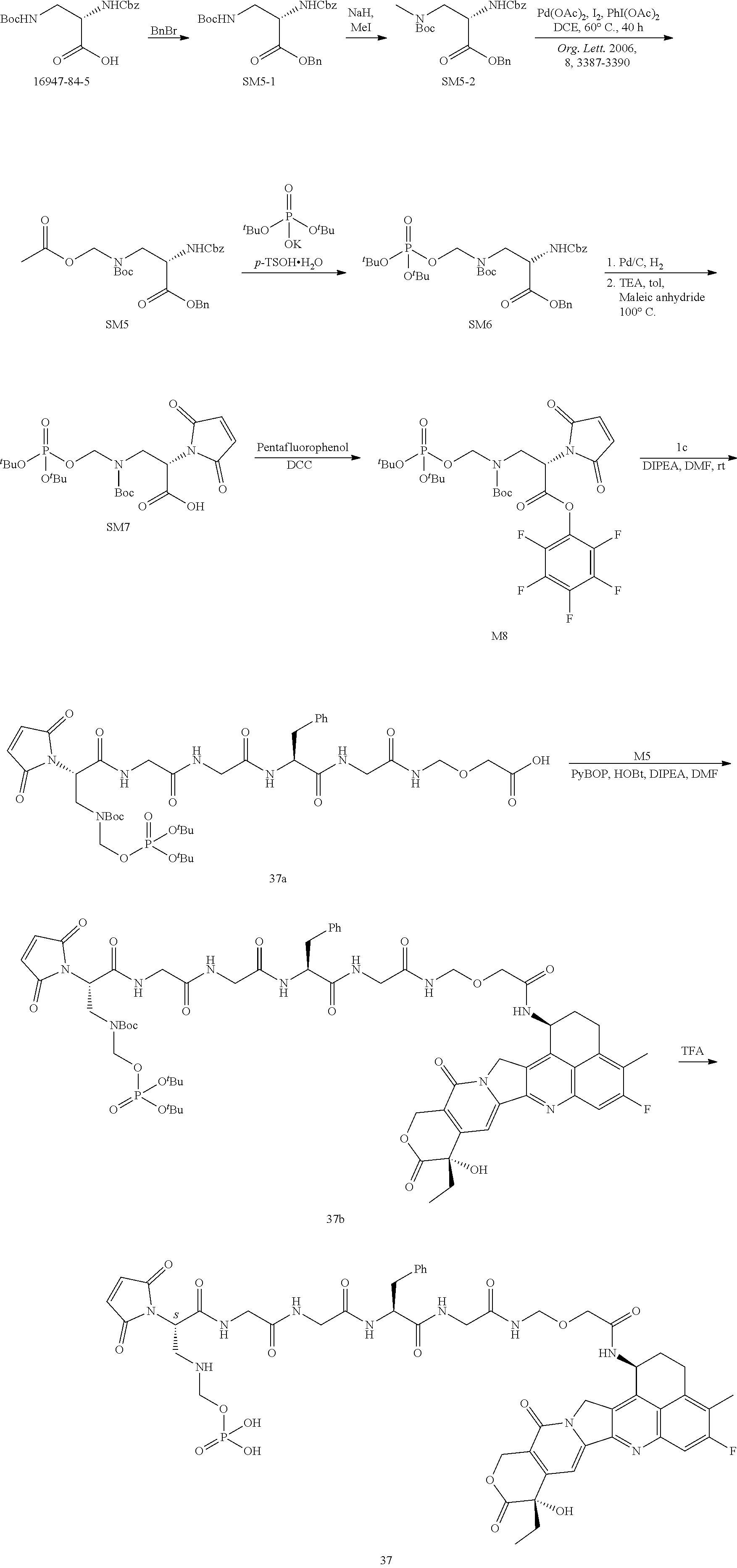

### Step 1: Compound SM5-1

In a 2000 mL single-necked flask, compound 16947-84-5 (100 g, 295 mmol, 1.0 eq), DIPEA (50 mL, 300 mmol), benzyl bromide (51.3 g, 300 mmol) and THF (1000 mL) were added, and reacted at room temperature for 12 h (monitored by TLC), and the insoluble material was filtered off. The solvent was directly removed from the reaction solution by rotary distillation under reduced pressure, and the residue was subjected to silica gel column chromatography (PE/EA=50/1-20/1-2/1) to obtain SM5-1 (110.1 g) at 87% yield; LC-MS: [M+H]⁺=429.2.

### Step 2: Compound SM5-2

In a 2000 mL single-necked flask, compound SM5-1 (100 g, 233.4 mmol, 1.0 eq) and THF (1000 mL) were added, cooled to 0° C. in an ice-water bath, NaH (37.4 g, 933.5 mmol) and Mel (132.5 g, 933.5 mmol) were added in batches, and a reaction was maintained at 0° C. for 24 h (monitored by TLC). The reaction was quenched by adding 500 mL of saturated NH₄C1 aqueous solution, extraction was performed three times with 500 mL of ethyl acetate, followed by drying of the organic phase with anhydrous sodium sulfate, and filtering. The solvent was directly removed from the filtrate by rotary distillation under reduced pressure, and the residue was subjected to silica gel column chromatography (PE/EA=100/1-50/1-10/1) to obtain SM5-2 (37.1 g); LC-MS: [M+H]⁺=443.3.

### Step 3: Compound SM5 (cf. Org. Lett., 2006, 8, 3387-3390.)

In a 1000 mL single-necked flask, compound SM5-2 (35 g, 79 mmol, 1.0 eq) and DCE (500 mL) were added, palladium diacetate (180 mg, 0.8 mmol), 12 (20 g, 79 mmol) and iodobenzene diacetate (40.8 g, 126.4 mmol) were added sequentially, and the temperature was raised to 60° C. to react for 40 h (monitored by TLC). The reaction was quenched by adding 500 mL of saturated aqueous sodium thiosulfate solution, extraction was performed three times with 500 mL of dichloromethane, followed by drying of the organic phase with anhydrous sodium sulfate, and filtering. The solvent was directly removed from the filtrate by rotary distillation under reduced pressure, and the residue was subjected to silica gel column chromatography (PE/EA=100/1-50/1-10/1) to obtain SM5 (28 g); LC-MS: [M+H]⁺=501.3.

### Step 4: Compound SM6

In a 500 mL single-necked flask, compound SM5 (25 g, 50 mmol, 1.0 eq), di-tert-butyl potassium phosphate (13.66 g, 55 mmol, 1.1 eq), p-toluenesulfonic acid monohydrate (951 mg, 5 mmol, 0.1 eq) and THF (200 mL) were added, and the reaction was carried out at room temperature for 1 h (monitored by TLC), and insoluble material was filtered off. The reaction solution was purified directly by preparative LC, and the preparation liquid was concentrated at 35° C. in a water bath under reduced pressure by water pump to remove acetonitrile, and lyophilized to obtain compound SM6 (15.1 g) at 46% yield; LC-MS: [M+H]⁺=651.4.

### Step 5: Compound SM7

SM6 (15 g, 23 mmol) and 100 mL of DMF were added into a 250 mL single-necked flask, and after dissolving, 15 g of 5% Pd/C was added in an ice-water bath, the atmosphere in the system was replaced by hydrogen three times, and a reaction was carried out at room temperature for 12 h. The Pd/C was removed by filtration, and then the solvent was removed by rotary distillation under reduced pressure with an oil pump, leaving a crude product ready for use.

In another 250 mL single-necked flask, add the above crude product and 100 mL of toluene, triethylamine (6.4 mL, 46 mmol) and maleic anhydride (2.4 g, 24 mmol), and after dissolution, raise to 100° C. to react for 2 h. Use HPLC to monitor the progress of the reaction, and when the reaction is completed, purify the reaction solution by HPLC, to obtain a preparation liquid. The preparation liquid was extracted with dichloromethane, washed with saturated sodium chloride solution, dried with anhydrous sodium sulfate, filtered, and concentrated to obtain 4.2 g of a solid at 36% yield; LC-MS: [M+H]⁺=507.3.

### Step 6: Compound M8

In a 100 mL single-necked flask, compound SM7 (4 g, 7.9 mmol, 1.0 eq), pentafluorophenol (1.6 g, 8.7 mmol, 1.1 eq), DCC (1.8 g, 8.7 mmol, 1.1 eq) and THF (60 mL) were added, and the reaction was carried out at room temperature for 1 h (monitored by TLC), and insoluble material was filtered off. The reaction solution was purified directly by preparative LC, and the preparation liquid was concentrated at 35° C. in a water bath under reduced pressure by water pump to remove acetonitrile, and lyophilized to obtain compound M8 (3.7 g) at 70% yield; LC-MS: [M+H]⁺=673.2.

### Step 7: Compound 37a

In a 25 mL single-necked flask, 1c (1 g, 2.36 mmol) was added, and after dissolution with 25 mL of DMF, DIPEA (430 uL, 2.6 mmol) was added, then M8 (1.2 g, 2.36 mmol) was added, and then the temperature was raised to room temperature to react for 1 h. The completion of the reaction was detected by HPLC, and the reaction solution was purified by high performance liquid chromatography to obtain a preparation liquid, which was lyophilized to obtain 488 mg of product; LC-MS: [M−H]⁻=911.0.

### Step 8: Compound 37b

37a (400 mg, 0.44 mmol), exatecan mesylate M5 (235 mg, 0.44 mmol), PyBOP (199 mg, 0.5 mmol), HOBt (69 mg, 0.5 mmol) and 10 mL of DMF were added to a 100 mL single-necked flask, DIPEA (218 uL, 1.32 mmol) was added in an ice-water bath, and the temperature was raised to room temperature to react for 2 h. After the reaction was complete as detected by HPLC, the reaction solution was purified by HPLC to obtain the preparation liquid of compound 37b, which was lyophilized to give 201 mg of the compound; LC-MS: [M+H]⁺=1329.6.

### Step 9: Compound 37

Compound 37b (130 mg, 0.098 mmol), zinc bromide (221 mg, 0.98 mmol) and 10 mL of nitromethane were added to a 25 mL single-necked flask and a reaction was carried out at 40° C. for 1 h. After the reaction was complete as detected by HPLC, the solvent was removed by concentration under reduced pressure, and a crude product was obtained. The crude product was purified by high performance liquid chromatography to obtain the product preparation liquid, and the preparation liquid was lyophilized to obtain 96 mg of solid; LC-MS: [M+H]⁺=1117.4.

### Example 43

### Synthesis of Compound 38

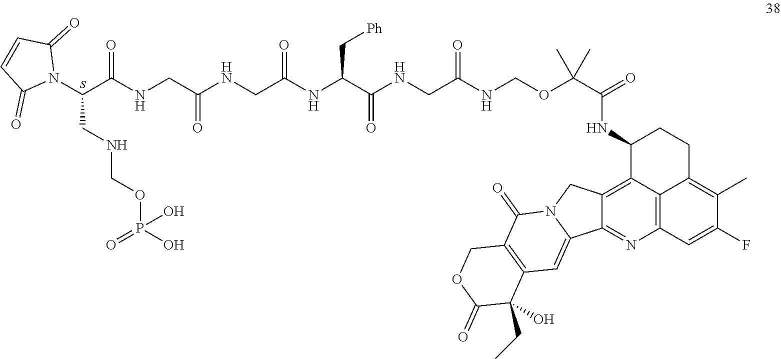

Using compounds M8 and 3c as starting materials and referring to the synthetic route of Example 42, compound 38 (51 mg) was obtained; LC-MS: [M+H]⁺=1145.6.

### Example 44

### Synthesis of Compounds 39 and 40

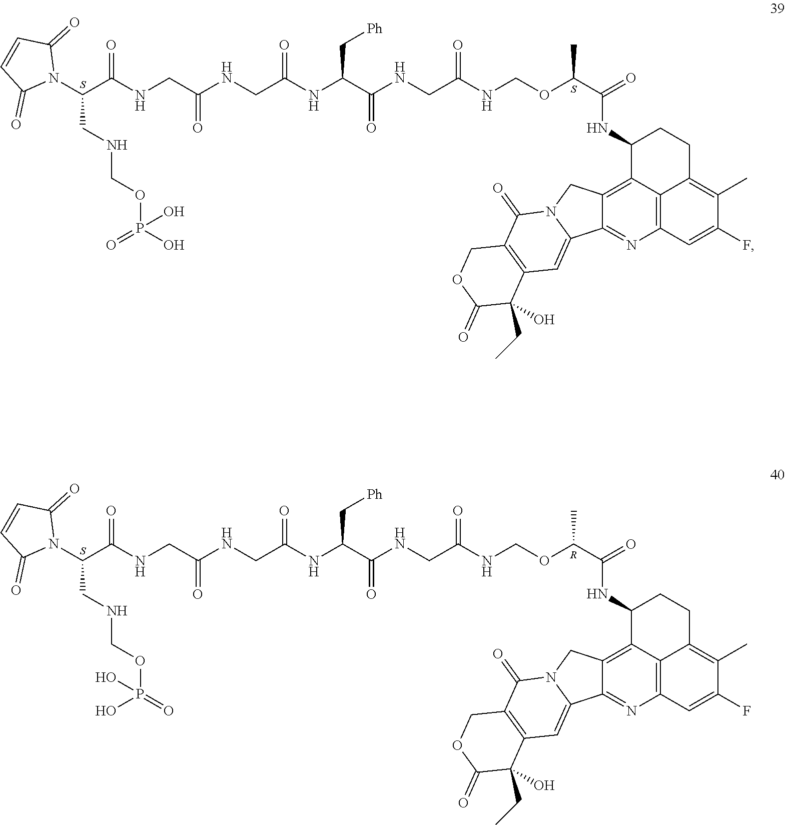

Using compounds M8 and 5c as starting materials and referring to the synthetic route of Example 42, 57 mg of compound 39 was obtained, LC-MS: [M+H]⁺=1131.4; 60 mg of compound 40 was obtained, LC-MS: [M+H]⁺=1131.4.

### Example 45

### Synthesis of Compounds 41 and 42

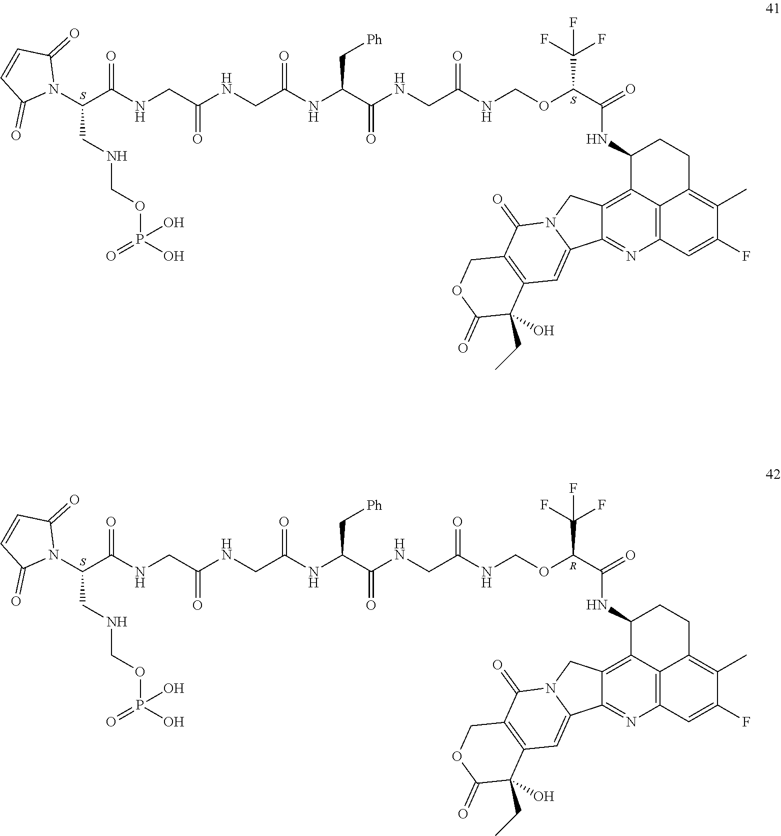

Using compounds M7 and 7c as starting materials, referring to the synthetic route of Example 42, 44 mg of compound 41 was obtained, LC-MS: [M+H]⁺=1185.3; 44 mg of compound 42 was obtained, LC-MS: [M+H]⁺=1185.3.

### Example 46

### Synthesis of Compounds 43 and 44

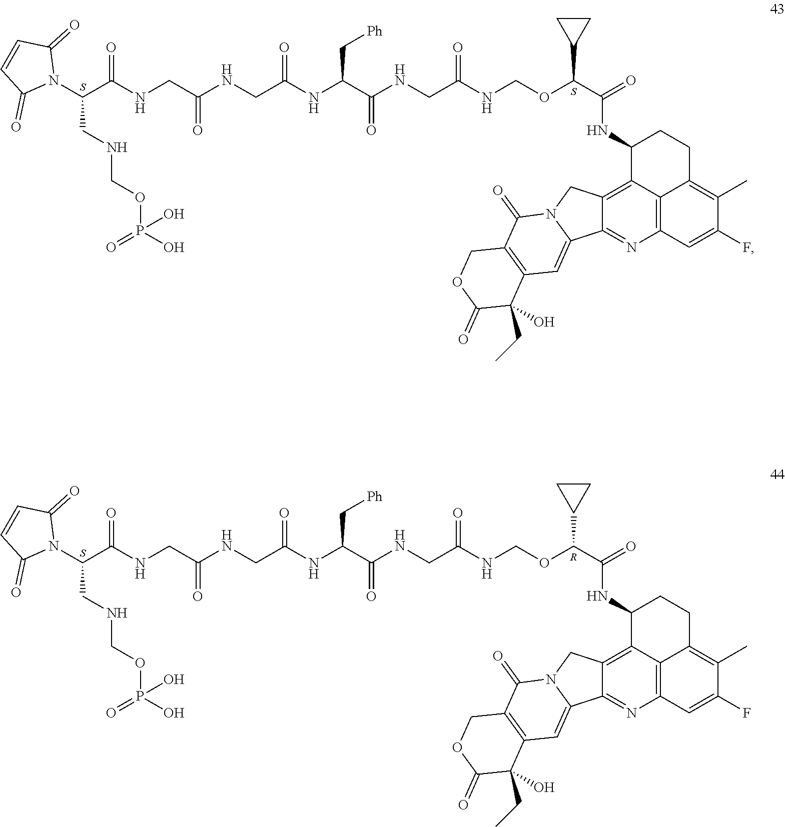

Using compounds M8 and 19c as starting materials and referring to the synthetic route of Example 42, 62 mg of compound 43 was obtained, LC-MS: [M+H]⁺=1157.4; 59 mg of compound 44 was obtained, LC-MS: [M+H]⁺=1157.4.

### Example 47 (Comparative Example)

### Synthesis of Compound 45

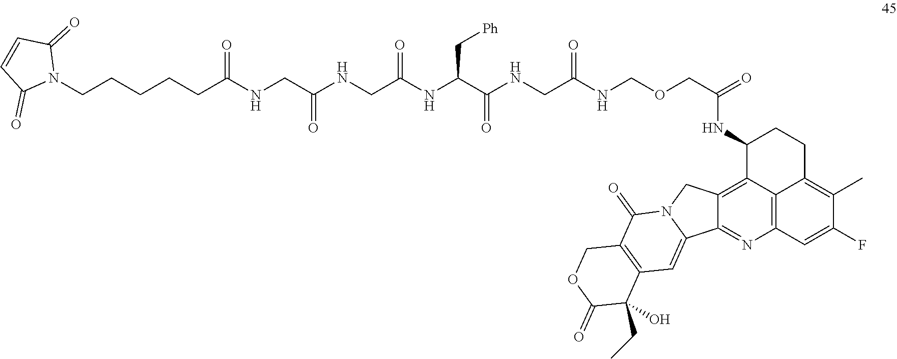 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

Compound 45 was synthesized by the method provided in Example 58 of patent “CN104755494A”.

### Example 48

### Synthesis of Compound 46

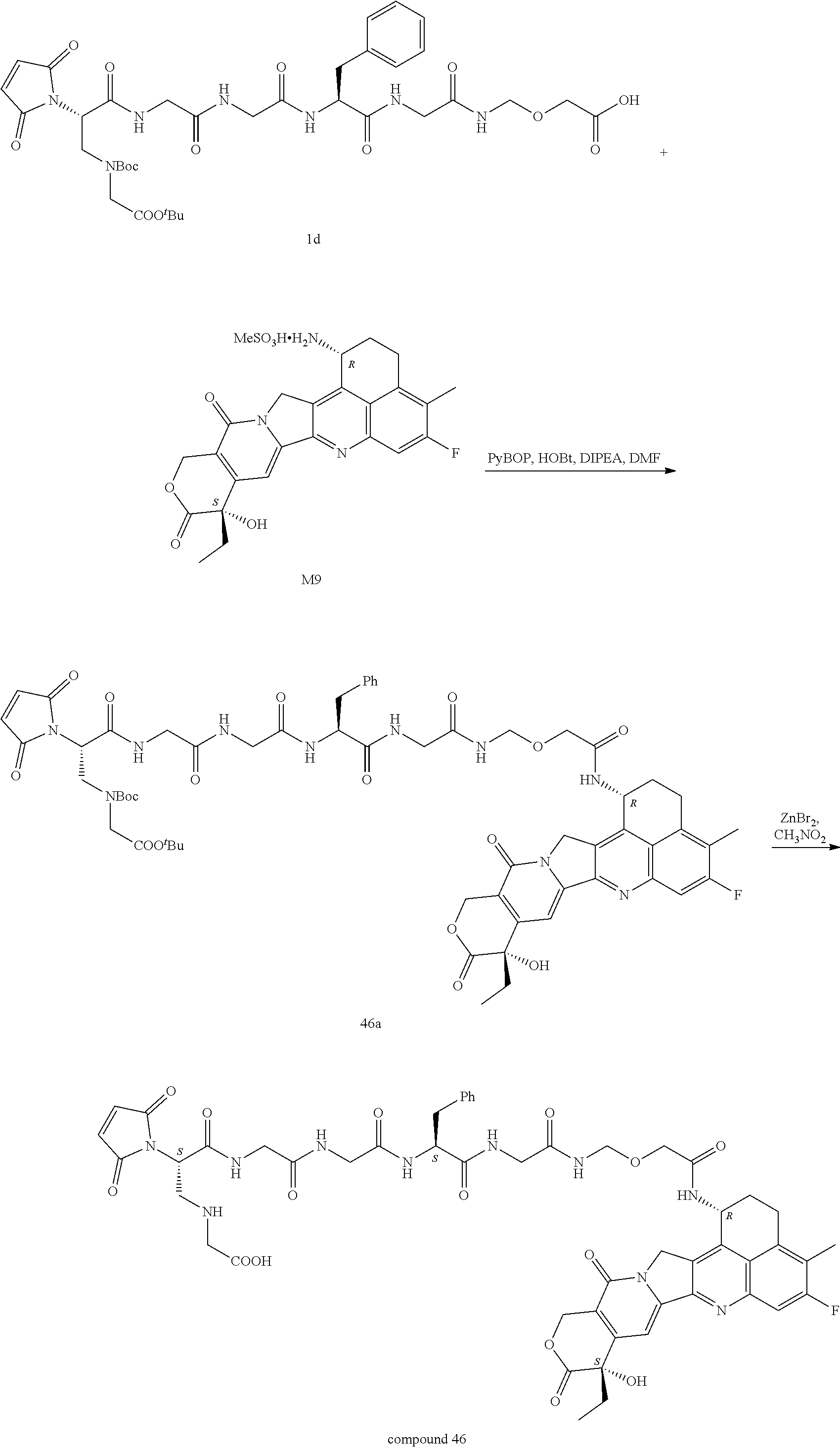

### Step 1: Compound 46a

1d (500 mg, 0.62 mmol), M9 (310 mg, 0.62 mmol), PyBOP (448 mg, 0.86 mmol), HOBt (116 mg, 0.86 mmol) and 15 mL of DMF were added to a 50 mL single-necked flask, DIPEA (378 uL, 2.29 mmol) was added in an ice-water bath, and the temperature was raised to room temperature to react for 2 h. After the completion of the reaction as detected by HIPLC, the reaction solution was purified by high performance liquid chromatography to obtain a preparation liquid, which was lyophilized to give 46a (210 mg); LC-MS: [M+H]⁺=1221.6.

### Step 2: Compound 46

46a (200 mg, 0.162 mmol), zinc bromide (736 mg, 3.26 mmol) and 10 mL of nitromethane were added to a 25 mL single-necked flask and a reaction was carried out at 40° C. for 1 h. After the reaction was complete as detected by HIPLC, the solvent was removed by concentration under reduced pressure, and a crude product was obtained. The crude product was purified by high performance liquid chromatography to obtain the product preparation liquid, and the preparation liquid was lyophilized to obtain solid compound 46 (120 mg); LC-MS: [M+H]⁺=1065.3.

### Example 49

### Synthesis of Compound 47

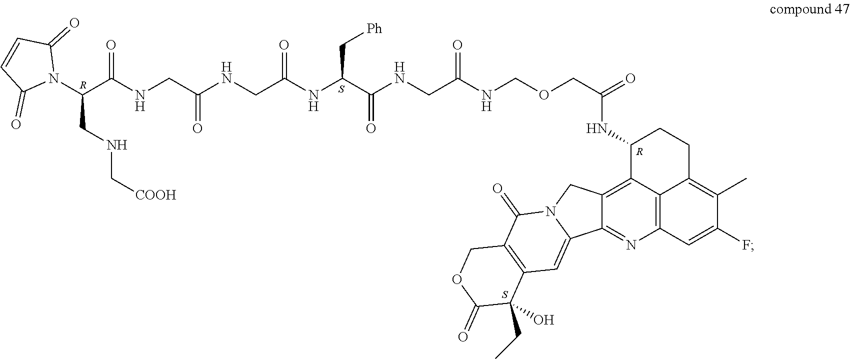

Referring to the synthetic route of Example 48, compound 47 (81 mg) was obtained; LC-MS: [M+H]⁺=1065.3.

### Example 50

### Synthesis of Compound 48A

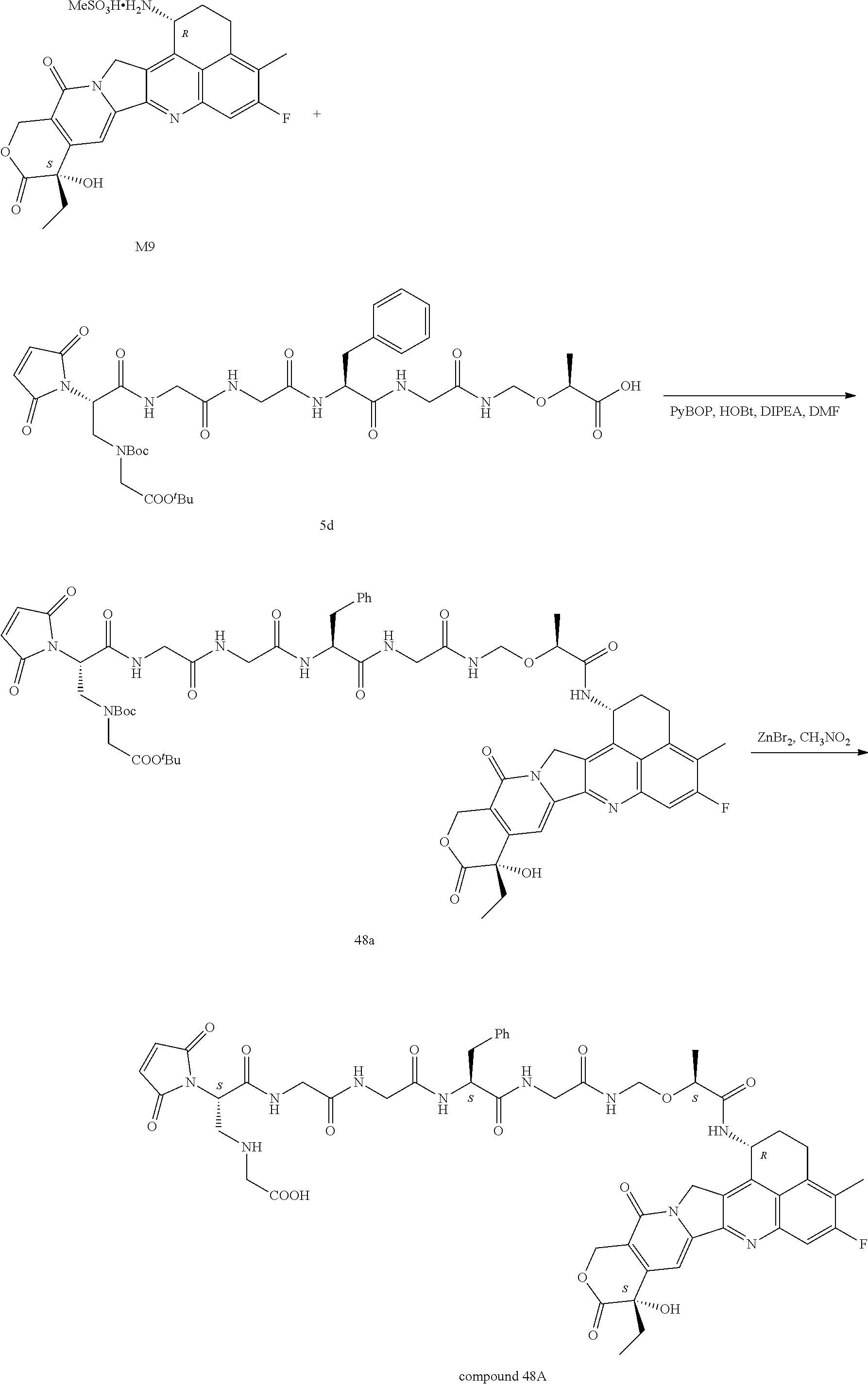

### Step 1: Compound 48a

Add 5d (1.66 g, 2.02 mmol, 1.0 eq), M9 (1.08 g, 2.02 mmol, 1.0 eq), PyBOP (1.58 g, 3.03 mmol, 1.5 eq), HOBt (0.41 g, 3.03 mmol, 1.5 eq) and DMF (40 mL) to a 100 mL single-necked flask. DIPEA (0.84 mL, 1.5 eq) was added in an ice-water bath, and the temperature was raised to room temperature to react for 2 h (monitored by HPLC). The reaction solution was directly purified by preparative LC, and the preparation liquid was concentrated at 35° C. in a water bath under reduced pressure by water pump to remove acetonitrile, and freeze-dried to obtain compound 48a (1.54 g), with a yield of 61%; LC-MS: [M+H]⁺=1235.4.

### Step 6: Compound 48A

Add compound 48a (1.0 g, 0.8 mmol, 1.0 eq) and 35 mL nitromethane into a 100 mL single-necked flask, and after dissolving, add zinc bromide (3.64 g, 16 mmol, 20.0 eq), react in an oil bath at 40° C. (preheated to stabilize) for 30 min, and perform concentration in a water bath at 45° C. under reduced pressure with a water pump to remove nitromethane, to obtain a yellow residue solid (monitored by HPLC). A preparation liquid was obtained by acid preparation. The preparation liquid was concentrated at 35° C. in a water bath under reduced pressure by a water pump to remove acetonitrile by spinning, and was lyophilized to obtain compound 48A (786 mg) with a yield of 90%.

### Example 51

### Synthesis of Compound 48B

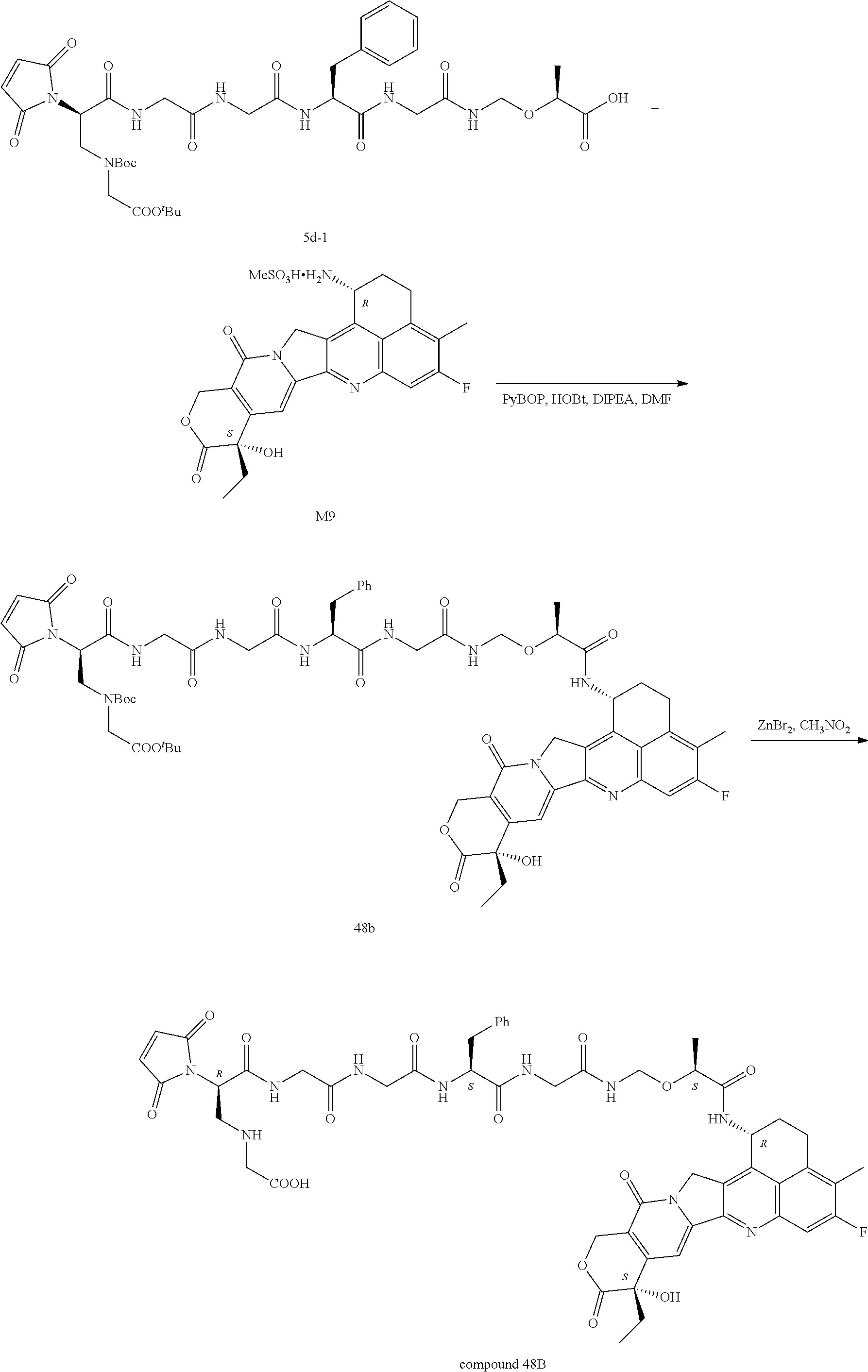

### Step 1: Compound 48b

Compound 5d-1 (200 mg, 0.24 mmol, 1.0 eq), M9 (127 mg, 0.24 mmol, 1.0 eq), PyBOP (187 mg, 0.36 mmol, 1.2 eq), HOBt (48 mg, 0.36 mmol, 1.2 eq), and DMF (6 mL) were added to a 25 mL single-necked flask. The temperature was lowered to 0-5° C. in an ice-water bath, DIPEA (62 mg, 0.48 mmol, 2.0 eq) was added, and then the temperature was raised to 20±5° C. to react for 2 h. The end of the reaction was detected by HIPLC. The reaction solution was purified directly by HIPLC preparation, and the product preparation liquid was collected and lyophilized to obtain compound 48b (150.2 mg); LC-MS: [M+H]⁺=1235.4.

### Step 2: Compound 48B

Compound 48b (100 mg, 0.081 mmol, 1.0 eq), ZnBr₂ (364 mg, 1.62 mmol, 20.0 eq) and CH₃NO₂ (10 mL) were added to a 25 mL single-necked flask in sequence, and then the temperature was raised to 40° C. to react for 0.5 h. The reaction was stopped, and the reaction solution was directly spin-dried at 45° C. under reduced pressure to obtain a yellow solid, taking samples to monitor the reaction by HPLC. The spin-dried solid was directly purified by HPLC preparation, and the product preparation liquid was collected and lyophilized to obtain compound 48B (70.0 mg); LC-MS: [M+H]⁺=1079.4.

### Example 52

### Synthesis of Compound 49A

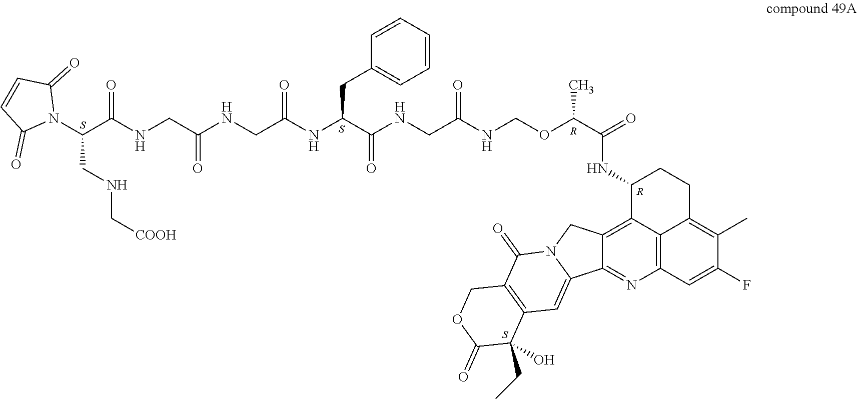

Referring to the route of Example 50, compound 49A (71 mg) was obtained; LC-MS: [M+H]⁺=1079.4.

### Example 53

### Preparation of Compound 49B

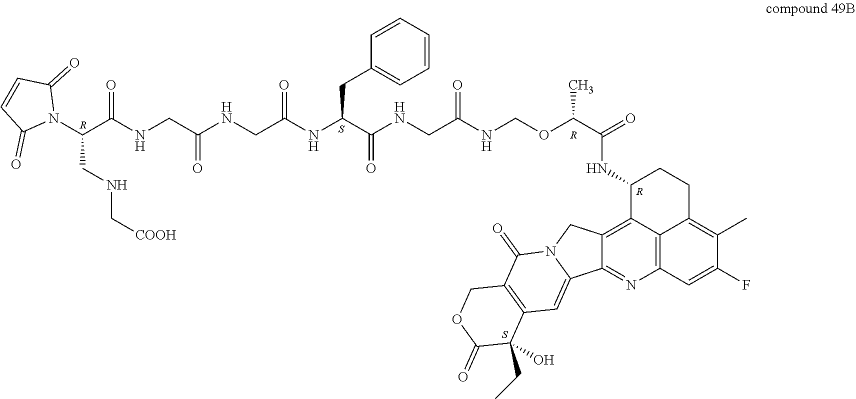

Referring to the synthetic route of Example 51, compound 49B (65 mg) was obtained; LC-MS: [M+H]⁺=1079.4.

### Example 54

### Synthesis of Compounds 50A and 50B

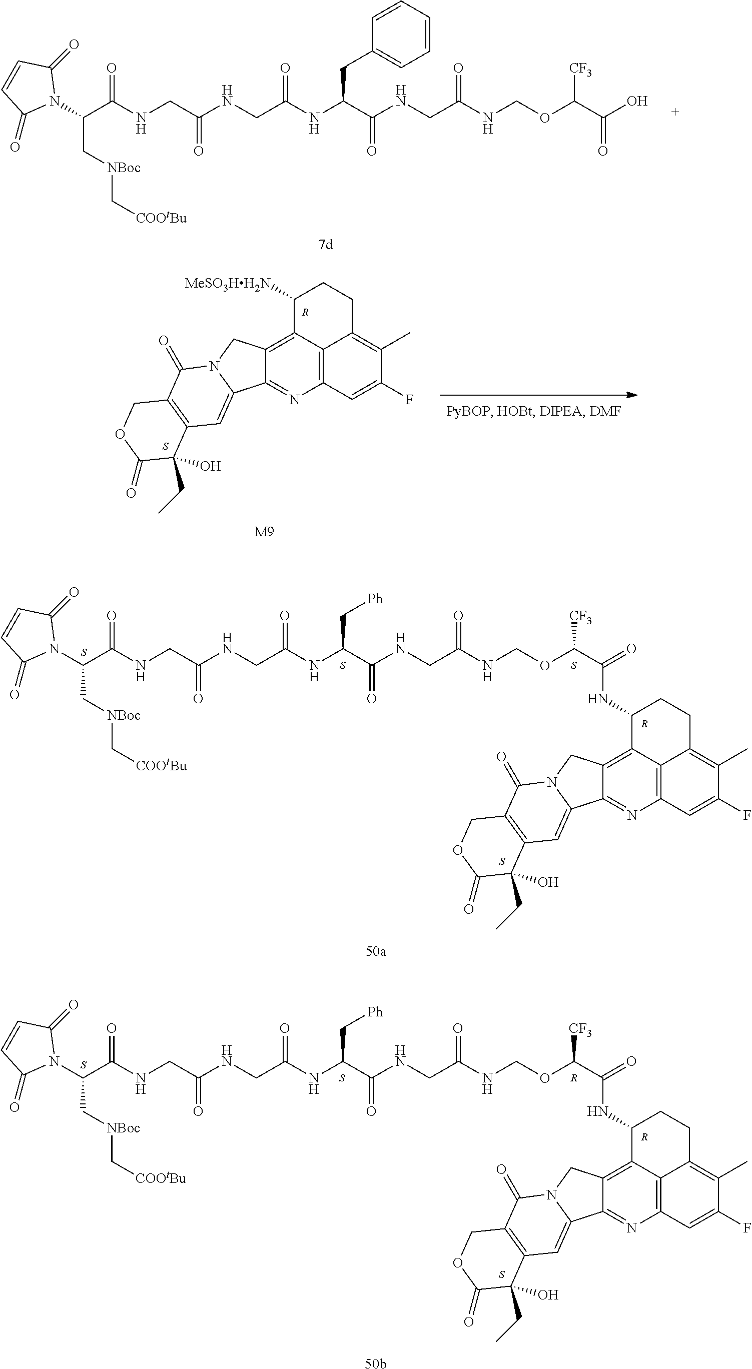

### Step 1: Compounds 50a and 50b

Add 7d (500 mg, 0.57 mmol), M9 (305 mg, 0.57 mmol), PyBOP (448 mg, 0.86 mmol), HOBt (116 mg, 0.86 mmol) and 15 mL DMF into a 50 mL single-necked flask, add DIPEA (378 uL, 2.29 mmol) in an ice-water bath, raise to room temperature and react for 2 h. After the completion of the reaction as detected by HIPLC, the reaction solution was purified by HIPLC to obtain the preparation liquids of compound 50a and compound 50b, and the preparation liquids were freeze-dried respectively to obtain 170 mg of compound 50a, LC-MS: [M+H]⁺=1289.46, and 202 mg of compound 50b, LC-MS: [M+H]⁺=1289.46.

### Step 2: Compound 50A

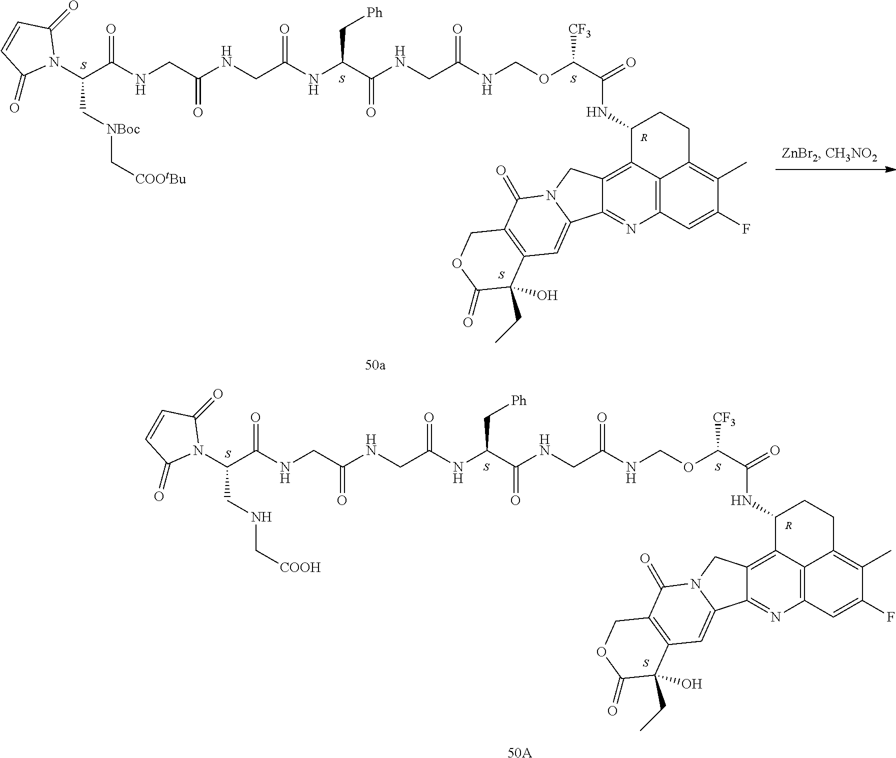

Add 50a (100 mg, 0.077 mmol), zinc bromide (349 mg, 1.55 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was freeze-dried to obtain 44 mg of solid.

### Step 3: Compound 50B

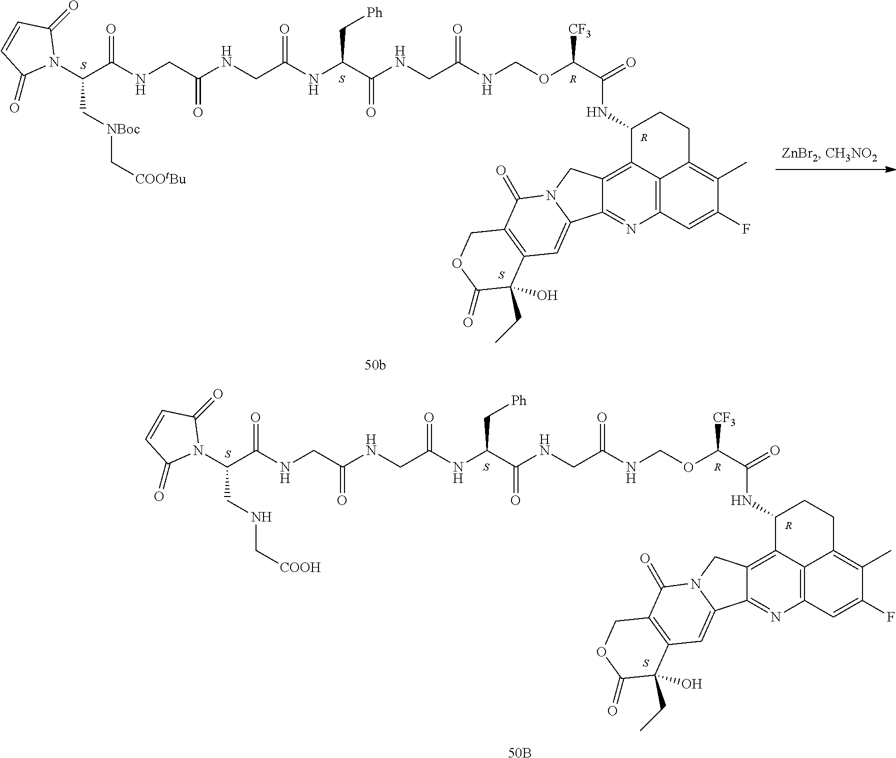

Add 50b (100 mg, 0.077 mmol), zinc bromide (349 mg, 1.55 mmol) and 5 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HIPLC to obtain a product preparation liquid, which was freeze-dried to obtain 45 mg of solid.

### Example 55

### Synthesis of Compounds 51A and 51B

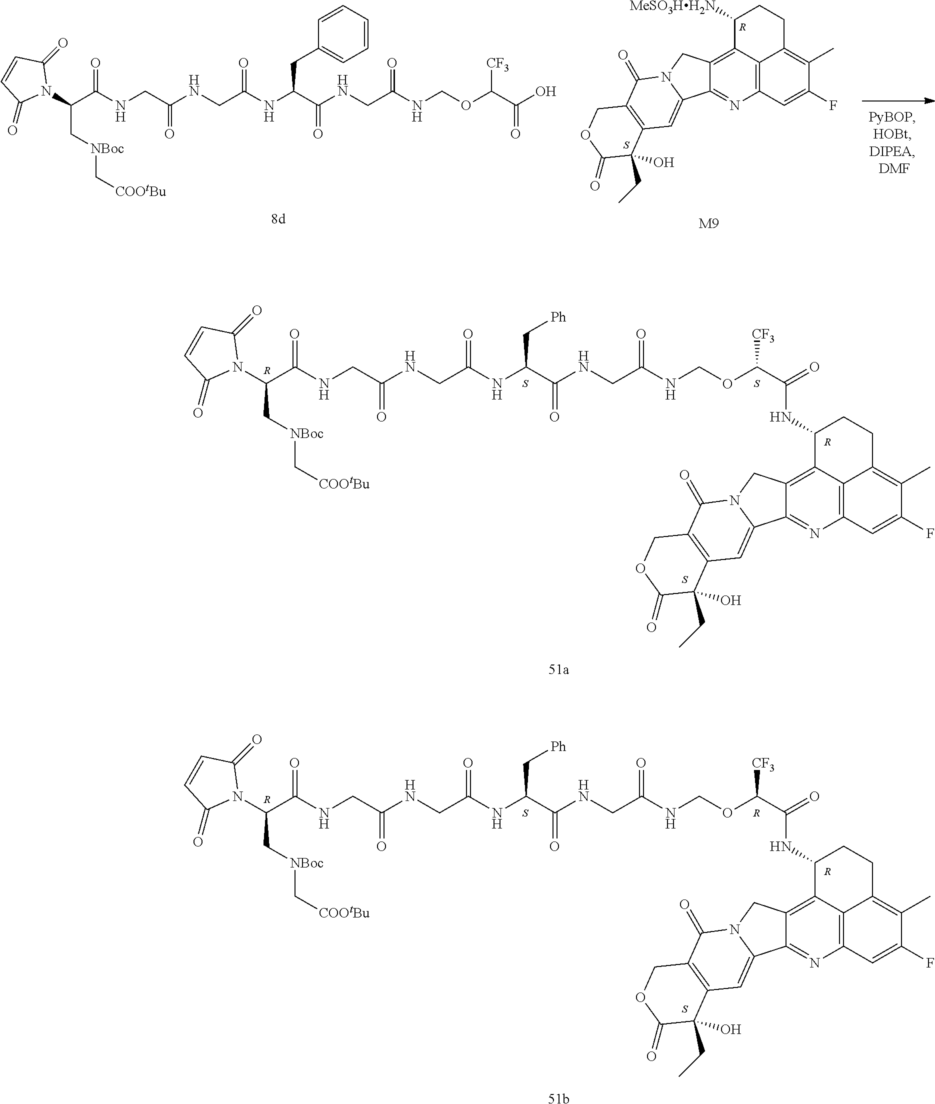

### Step 1: Compound 51a and Compound 51b

Add 8d (500 mg, 0.57 mmol), M9 (305 mg, 0.57 mmol), PyBOP (448 mg, 0.86 mmol), HOBt (116 mg, 0.86 mmol) and 15 mL DMF into a 50 mL single-necked flask, add DIPEA (378 uL, 2.29 mmol) in an ice-water bath, raise to room temperature and react for 2 h. After the reaction was complete as detected by HIPLC, the reaction solution was purified by HPLC to obtain preparation liquids of compound 51a and compound 51b, which were respectively lyophilized to obtain 190 mg of compound 51a and 186 mg of compound 51b. LC-MS of compound 51a: [M+H]⁺=1289.47; LC-MS of compound 51b: [M+H]⁺=1289.47.

### Step 2: Compound 51A

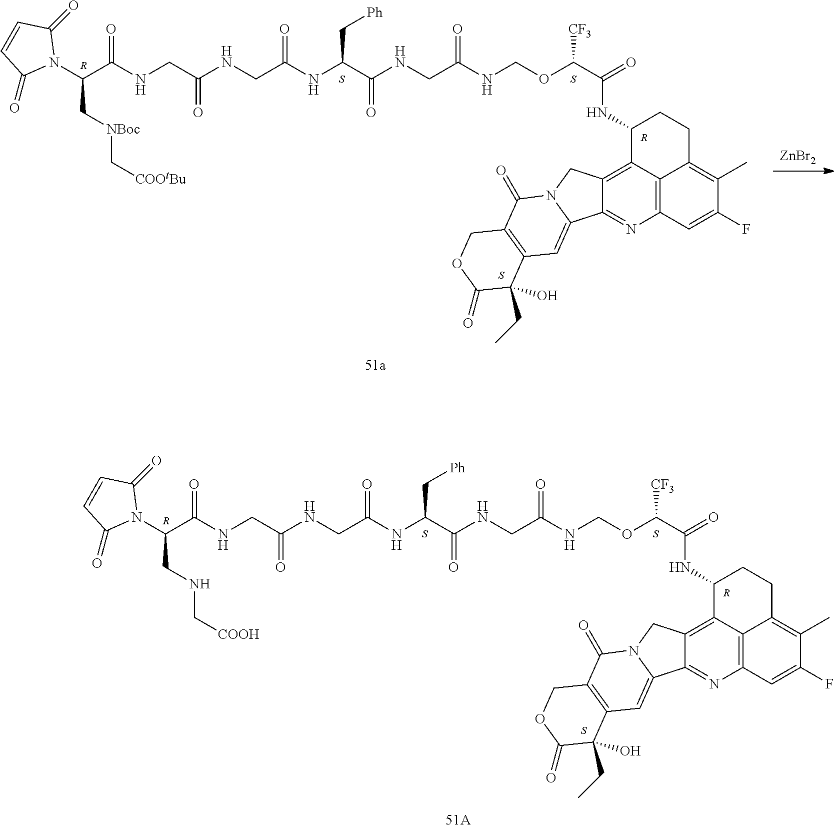

Add compound 51a (100 mg, 0.077 mmol), zinc bromide (349 mg, 1.55 mmol) and 5 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HPLC to obtain a product preparation liquid, which was lyophilized to obtain 39 mg of solid.

### Step 3: Compound 51B

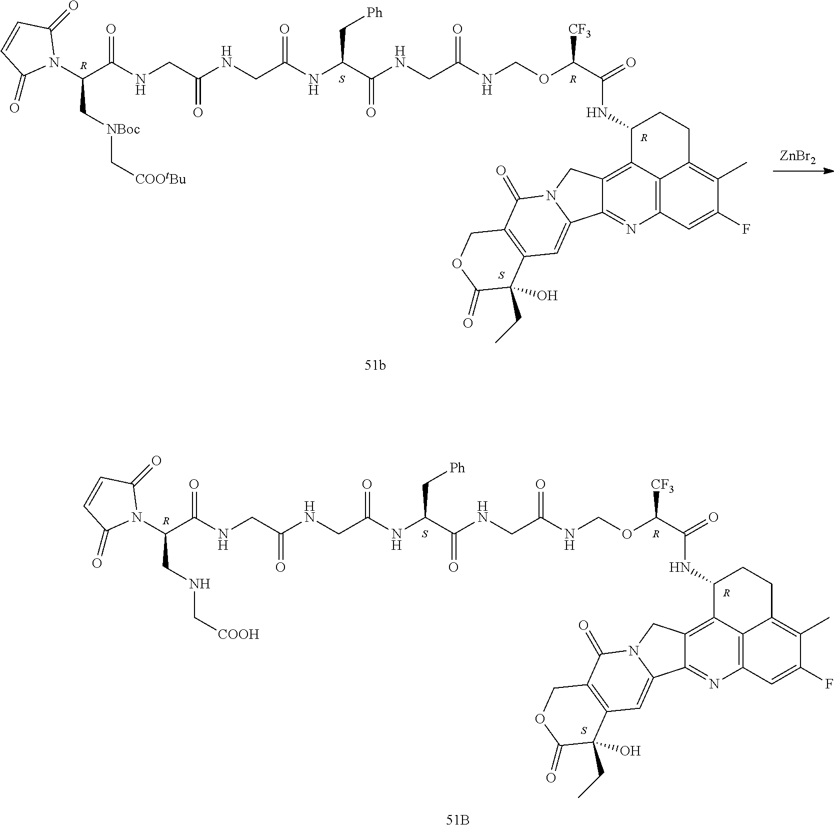

Add compound 51b (100 mg, 0.077 mmol), zinc bromide (349 mg, 1.55 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was freeze-dried to obtain 60 mg of solid.

### Example 56

### Synthesis of Compound 52A

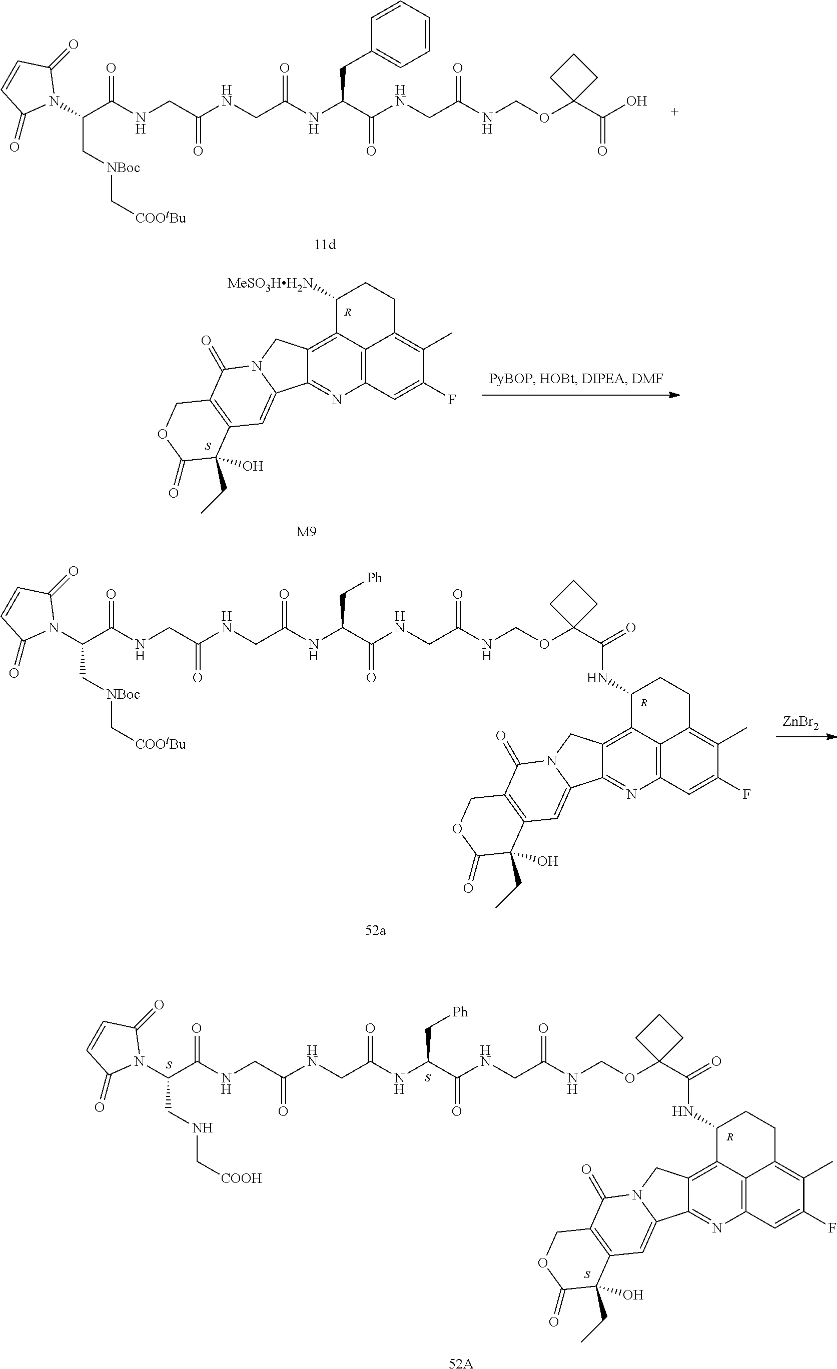

### Step 1: Compound 52a

Add 11d (800 mg, 0.96 mmol), M9 (480 mg, 0.96 mmol), PyBOP (500 mg, 0.96 mmol), HOBt (208 mg, 0.96 mmol) and 30 mL DMF into a 50 mL single-necked flask, add DIPEA (660 uL, 4.0 mmol) in an ice-water bath, raise to room temperature and react for 4 h. After the completion of the reaction as detected by HPLC, the reaction solution was purified by HPLC to obtain a preparation liquid of compound 52a, which was lyophilized to obtain 52a (388 mg); LC-MS: [M+H]⁺=1261.4.

### Step 2: Compound 52A

Add 52a (150 mg, 0.12 mmol), zinc bromide (532 mg, 2.4 mmol) and 10 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain solid compound 52A (79 mg); LC-MS: [M+H]⁺=1105.4.

### Example 57

### Synthesis of Compound 52B

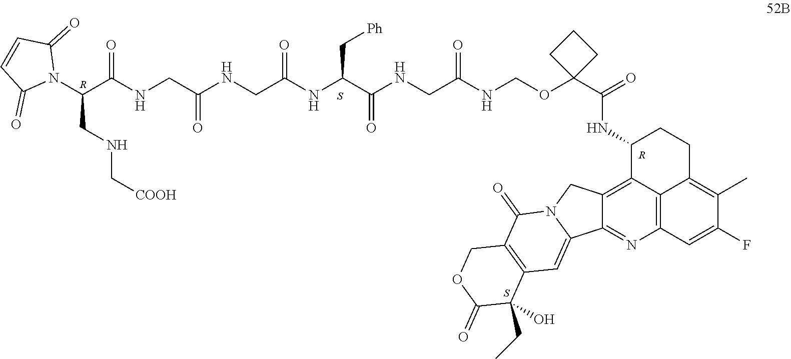

Referring to the synthetic route of Example 56, compound 52B (50 mg) was obtained. LC-MS: [M+H]⁺=1105.4.

### Example 58

### Synthesis of Compound 53A

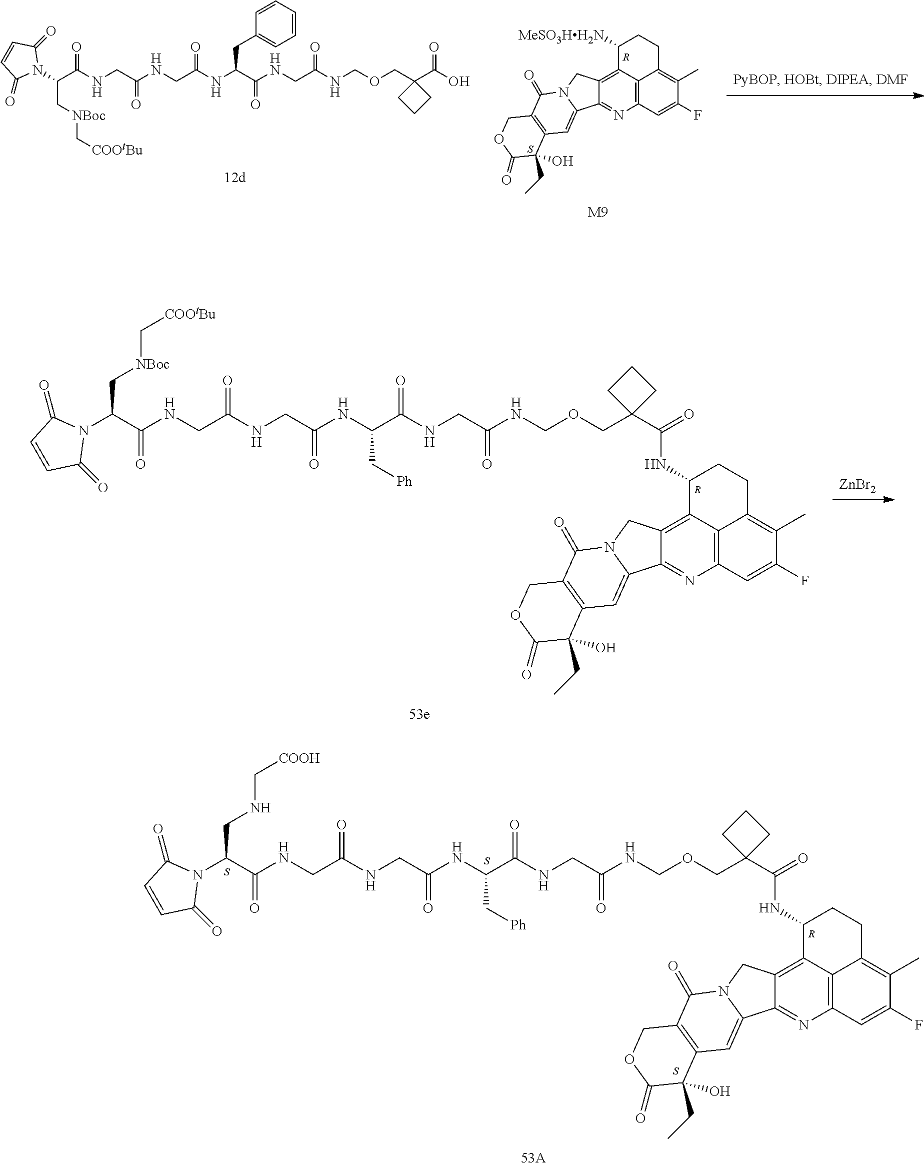

### Step 1: Compound 53a

Add 12d (400 mg, 0.47 mmol), M9 (240 mg, 0.47 mmol), PyBOP (250 mg, 0.47 mmol), HOBt (101 mg, 0.47 mmol) and 15 mL DMF into a 50 mL single-necked flask, add DIPEA (330 uL, 2.0 mmol) in an ice-water bath, raise to room temperature and react for 3 h. After the completion of the reaction as detected by HIPLC, the reaction solution was purified by HIPLC to obtain a preparation liquid of compound 53a, which was lyophilized to obtain 53a (200 mg); LC-MS: [M+H]⁺=1275.4.

### Step 2: Compound 53A

Add 53a (100 mg, 0.08 mmol), zinc bromide (360 mg, 1.6 mmol) and 5 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HPLC to obtain a product preparation liquid, which was lyophilized to obtain solid compound 53A (51 mg); LC-MS: [M+H]⁺=1119.4.

### Example 59

### Synthesis of Compound 53B

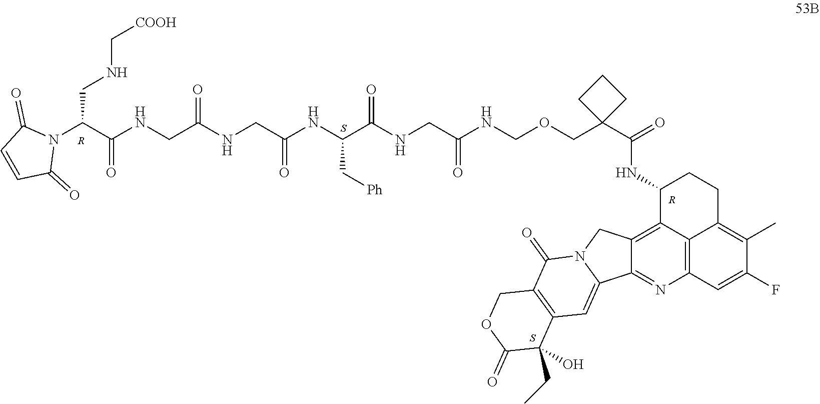

Referring to the synthetic route of Example 58, compound 53B (50 mg) was obtained; LC-MS: [M+H]⁺=1119.4.

### Example 60

### Synthesis of Compounds 54A and 54B

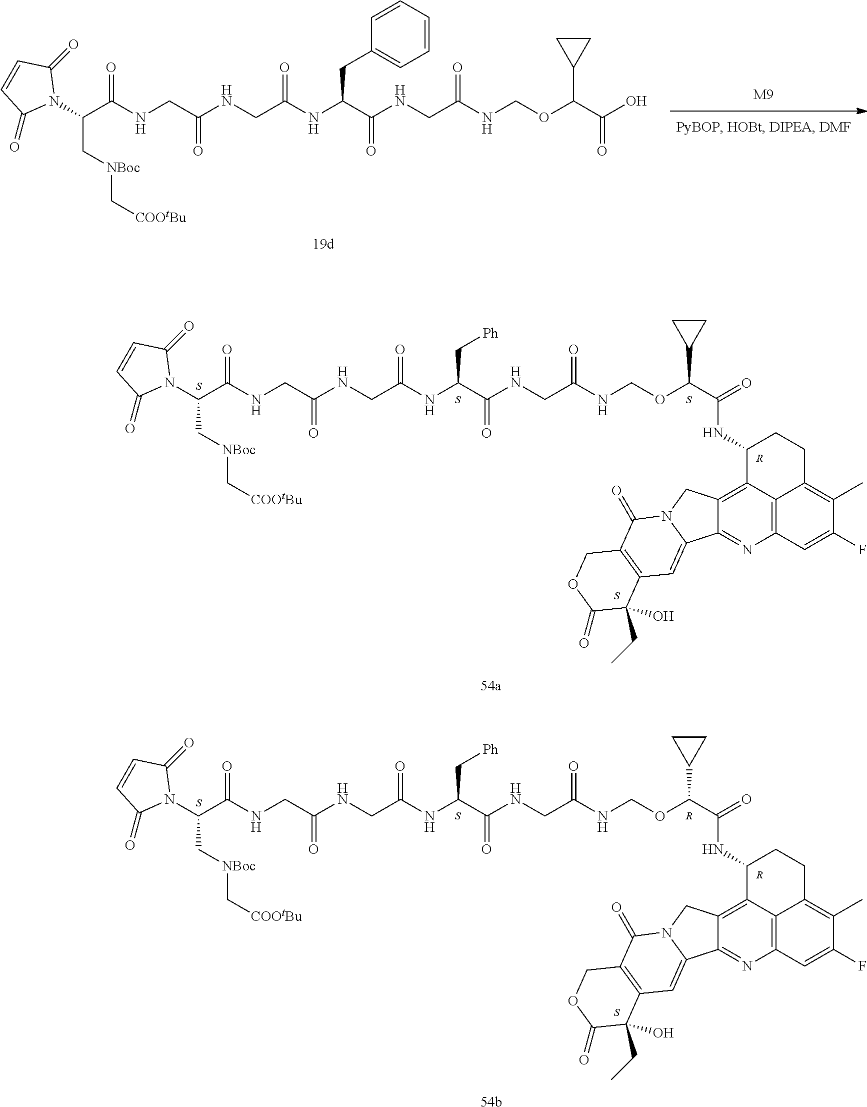

### Step 1: Compounds 54a and 54b

Add 19d (500 mg, 0.59 mmol), M9 (317 mg, 0.59 mmol), PyBOP (339 mg, 0.65 mmol), HOBt (88 mg, 0.86 mmol) and 10 mL DMF into a 50 mL single-necked flask, add DIPEA (292 uL, 1.77 mmol) in an ice-water bath, raise to room temperature and react for 2 h. After the completion of reaction as detected by HIPLC, the reaction solution was purified by HPLC to obtain the preparation liquids of compounds 54a and 54b. The preparation solutions were lyophilized separately to obtain 103 mg of compound 54a, LC-MS: [M+H]⁺=1261.5, and 111 mg of compound 54b, LC-MS: [M+H]⁺=1261.5.

### Step 2: Compound 54A

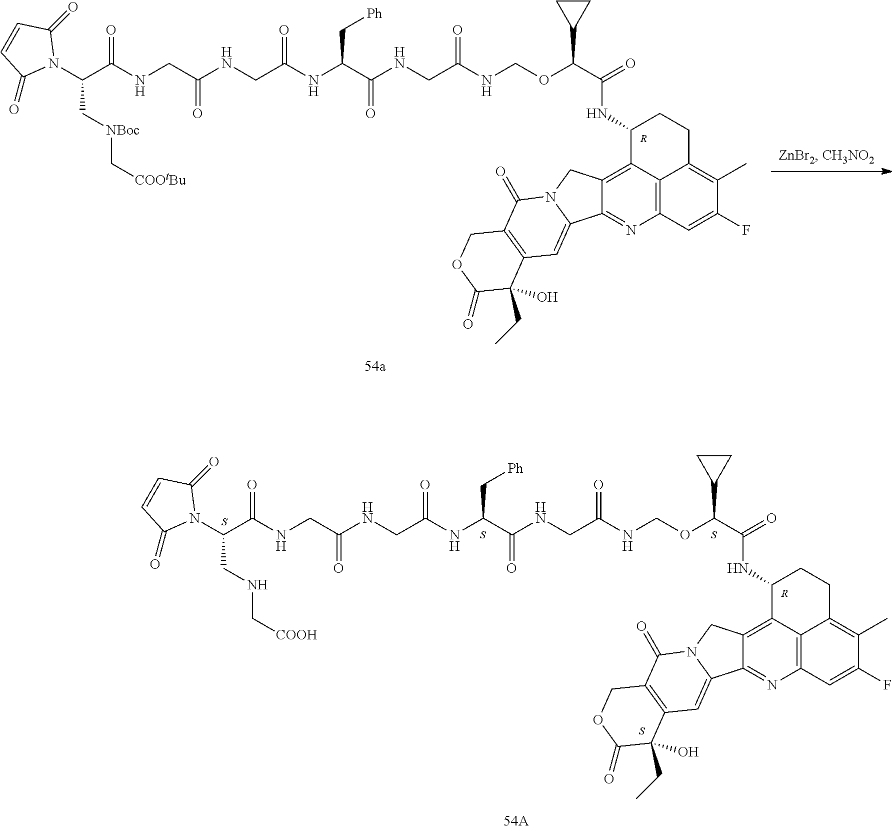

Add 54a (100 mg, 0.079 mmol), zinc bromide (357 mg, 1.59 mmol) and 5 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HPLC to obtain a product preparation liquid, which was lyophilized to obtain 61 mg of solid; LC-MS: [M+H]⁺=1105.4.

### Step 3: Compound 54B

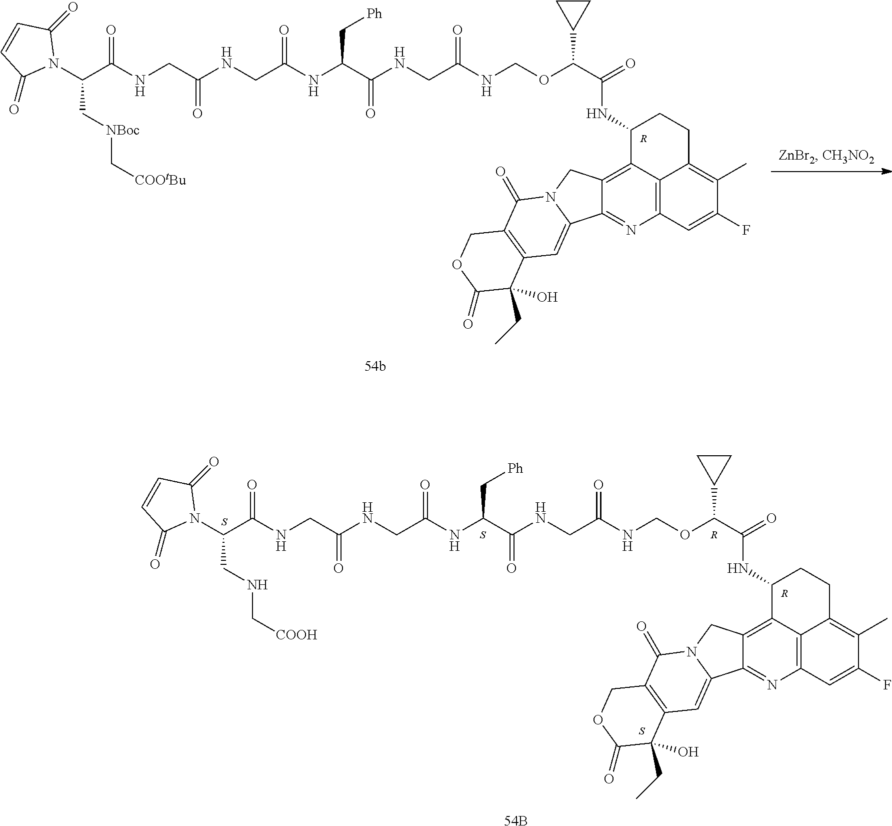

Add 54b (100 mg, 0.079 mmol), zinc bromide (357 mg, 1.59 mmol) and 5 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HIPLC to obtain a product preparation liquid, which was lyophilized to obtain 57 mg of solid; LC-MS: [M+H]⁺=1105.4.

### Example 61

### Synthesis of Compounds 55A and 55B

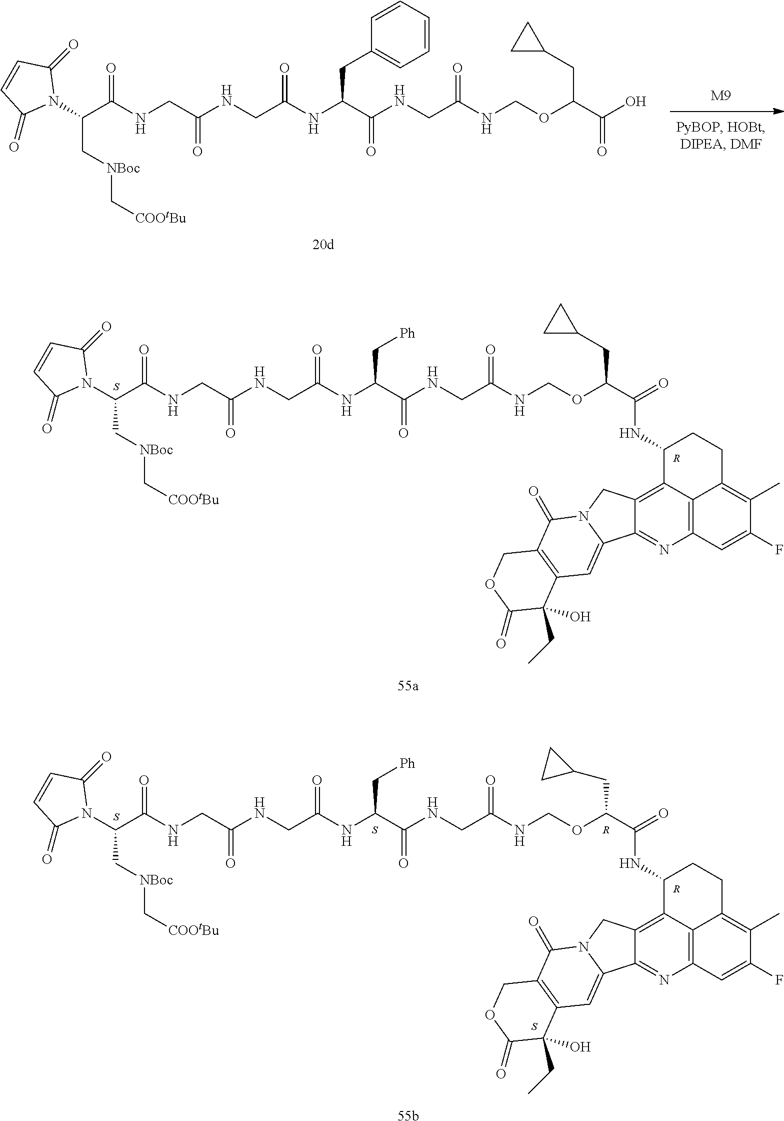

### Step 1: Compounds 55a and 55b

Add 20d (400 mg, 0.47 mmol), M9 (250 mg, 0.47 mmol), PyBOP (223 mg, 0.56 mmol), HOBt (83 mg, 0.56 mmol) and 10 mL DMF into a 50 mL single-necked flask, add DIPEA (248 uL, 1.5 mmol) in an ice-water bath, raise to room temperature and react for 2 h. After the reaction was complete as detected by HIPLC, the reaction solution was purified by HPLC to obtain the preparation liquids of compounds 55a and 55b. The preparation liquids were separately freeze-dried to obtain 100 mg of compound 55a, LC-MS: [M+H]⁺=1275.5, and 101 mg of compound 55b, LC-MS: [M+H]⁺=1275.5.

### Step 2: Compound 55A

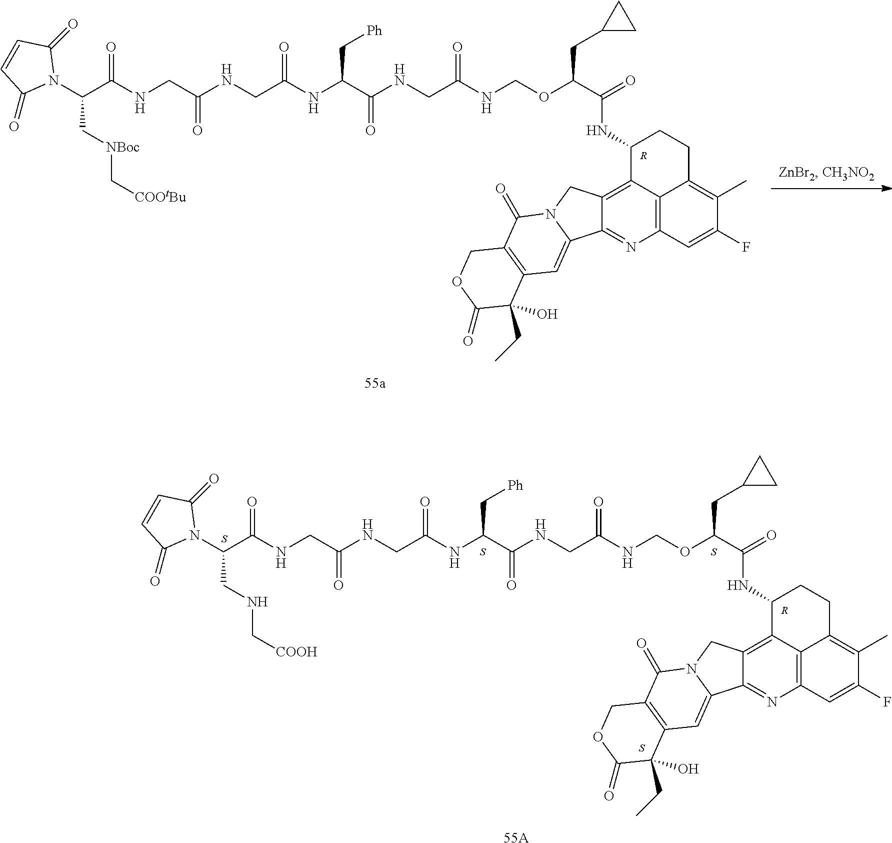

Add 55a (100 mg, 0.078 mmol), zinc bromide (352 mg, 1.57 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain 42 mg of solid; LC-MS: [M+H]⁺=1119.4.

### Step 3: Compound 55B

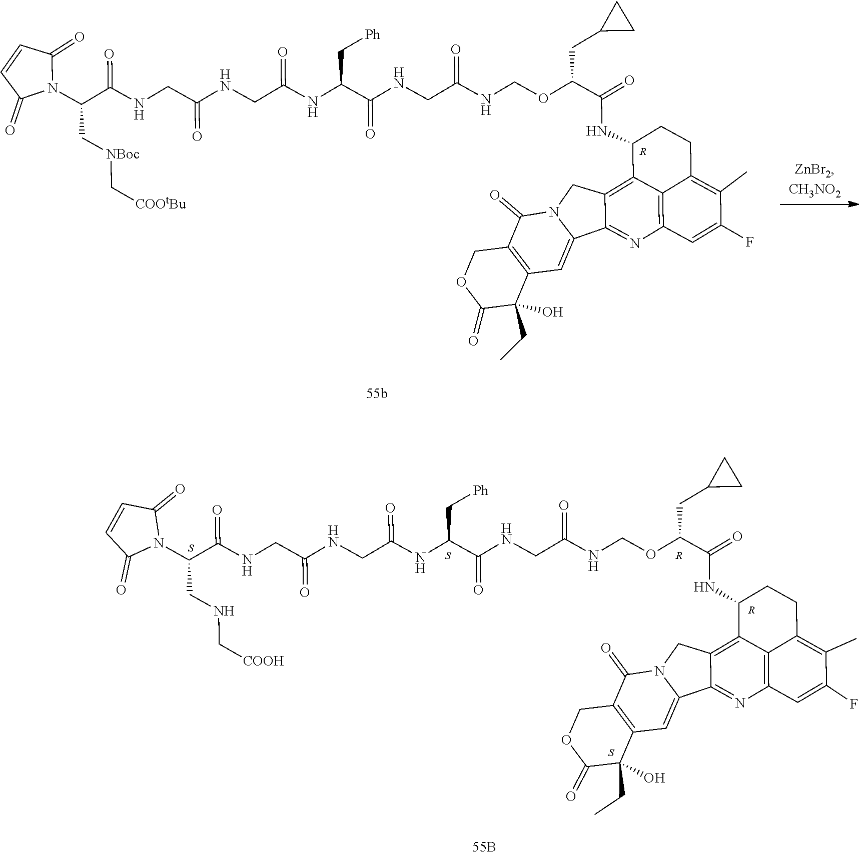

Add 55b (100 mg, 0.079 mmol), zinc bromide (357 mg, 1.59 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain 45 mg of solid; LC-MS: [M+H]⁺=1119.4.

### Example 62

### Synthesis of Compound 56

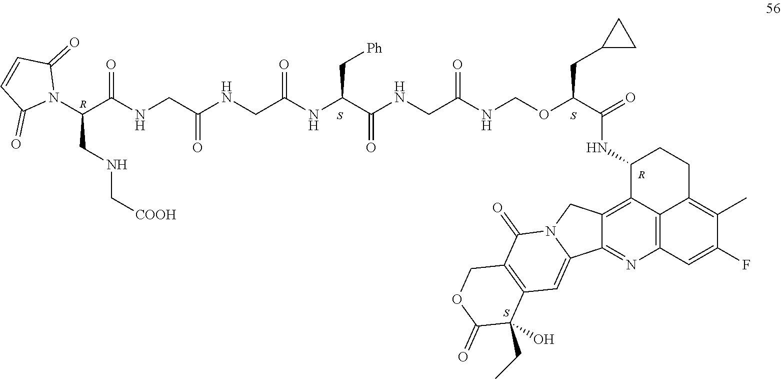

Referring to the synthetic route of Example 60, compound 56 (50 mg) was obtained; LC-MS: [M+H]⁺=1119.3.

### Example 63

### Synthesis of Compound 57

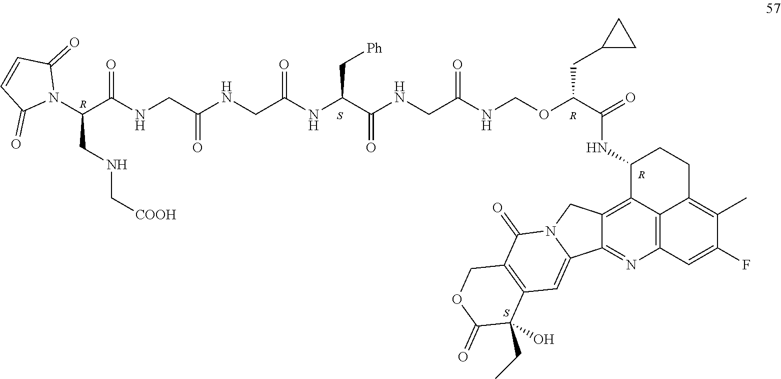

Referring to the synthetic route of Example 61, compound 57 (50 mg) was obtained; LC-MS: [M+H]⁺=1119.4.

### Example 64

### Synthesis of Compound 58

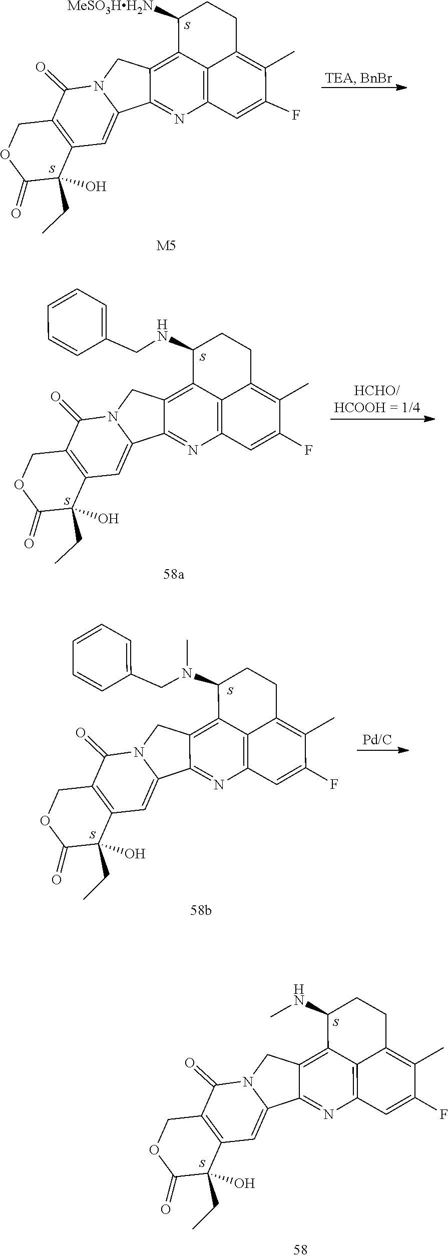

### Step 1: Synthesis of Compound 58a

Add 400 mL of DMF to exatecan mesylate M5 (15 g, 28 mol, prepared by the method disclosed in patent application “EP0737683A1”), cool in an ice-water bath to 0° C., add triethylamine dropwise, and adjust the pH to 7-8. Benzyl bromide (9.6 g, 56 mmol) was added dropwise in an ice-water bath, the temperature was raised to room temperature (25° C.) to react for 1 hour, completion of the reaction was detected by TLC, the reaction solution was concentrated under reduced pressure, the crude product obtained was purified by preparative high-performance liquid chromatography (acetonitrile/purified water system), the target peak was collected, the acetonitrile was removed under reduced pressure, then lyophilization was performed, to obtain about 11 g of compound 58a, a yellow solid, with a yield of about 74%, MS m/z: [M+H]⁺ 526.3.

### Step 2: Synthesis of Compound 58b

At room temperature, compound 58a (11 g, 21 mol) and 120 mL of formic acid for dissolution were sequentially added in a 250 mL single-necked flask, 30 mL of formaldehyde (40% aqueous solution) was added to the resulting bright yellow solution, and the temperature was raised to 50° C. to react for 1 h. When the reaction was complete as detected by TLC, the temperature was cooled down to room temperature, and the reaction solution was purified by preparative high-performance liquid chromatography (acetonitrile/purified water system), and the target peak was collected. After removing acetonitrile under reduced pressure, lyophilization was performed to give about 4.5 g of compound 58b, a yellow powdery solid, yield about 40%, MS m/z: [M+H]⁺ 540.6.

### Step 3: Synthesis of Compound 58

At room temperature, add compound 58b (2.3 g, 4.3 mol) in a 250 mL single-necked flask, add 100 mL of DMF to dissolve, add 2.3 g of 5% Pd/C to the resulting bright yellow solution, use a hydrogen balloon to replace the atmosphere in the system, and maintain the reaction at room temperature for 1.5 hours. Completion of the reaction was then detected by HIPLC, Pd/C was removed by filtration, and the resulting reaction solution was concentrated, and then purified by preparative high-performance liquid chromatography (acetonitrile/purified water system). The target peak was collected, and after removing acetonitrile under reduced pressure, lyophilization was performed to obtain about 1.0 g of compound 58, a yellow powdery solid with about 52% yield, MS m/z: [M+H]⁺ 450.5.

### Example 65

### Synthesis of Compound 59

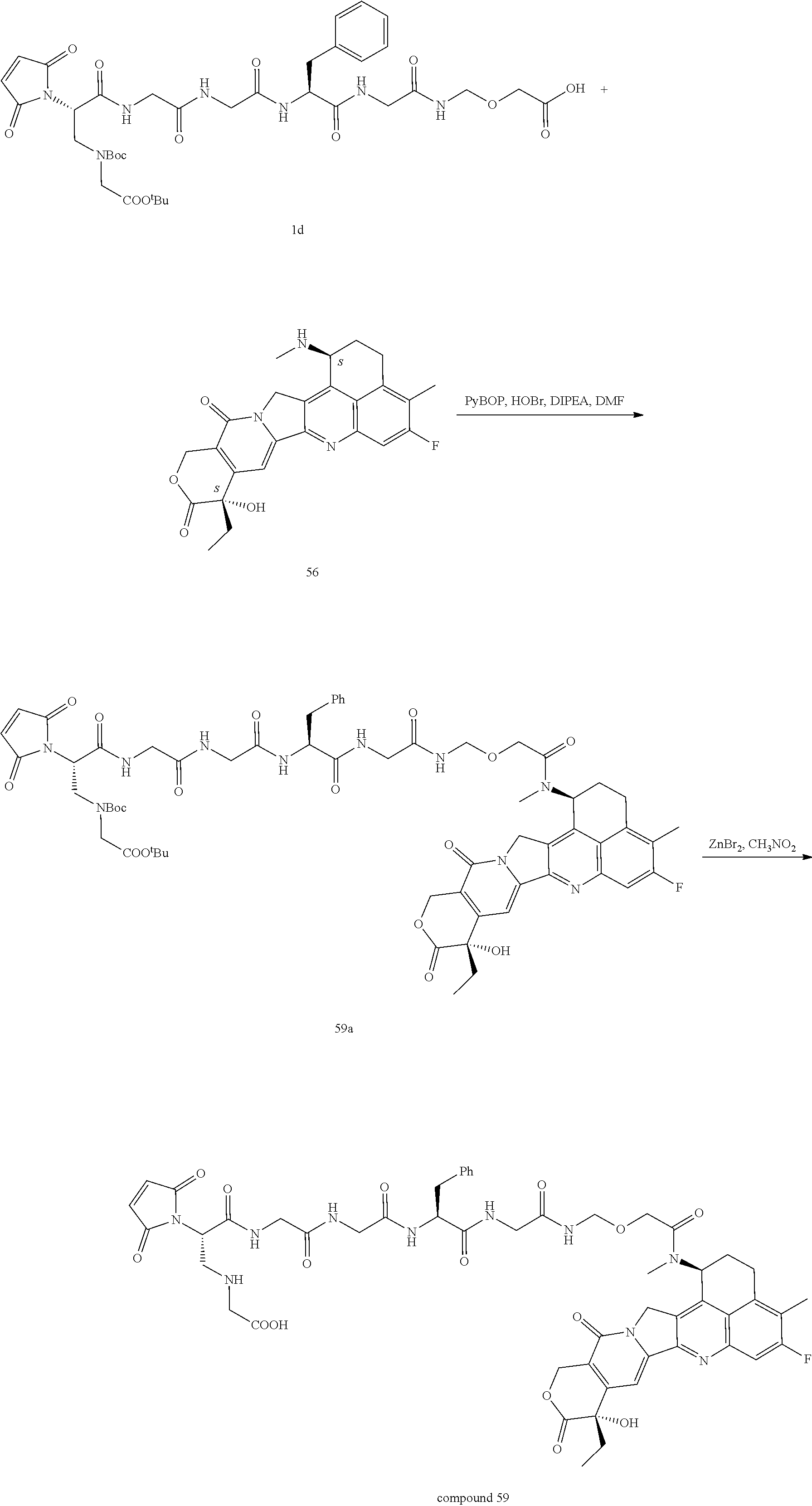

### Step 1: Compound 59a

Add 1d (500 mg, 0.62 mmol), 58 (279 mg, 0.62 mmol), PyBOP (448 mg, 0.86 mmol), HOBt (116 mg, 0.86 mmol) and 15 mL of DMF into a 50 mL single-necked flask, add DIPEA (378 uL, 2.29 mmol) in an ice-water bath, raise to room temperature and react for 2 h. After the completion of the reaction as detected by HIPLC, the reaction solution was purified by HIPLC to obtain a preparation liquid, which was lyophilized to obtain 59a (166 mg); LC-MS: [M+H]⁺=1235.6.

### Step 2: Compound 59

59a (100 mg, 0.081 mmol), zinc bromide (368 mg, 1.63 mmol) and 10 mL of nitromethane were added to a 25 mL single-necked flask and a reaction was carried out at 40° C. for 1 h. After the reaction was complete as detected by HIPLC, the solvent was removed by concentration under reduced pressure, and a crude product was obtained. The crude product was purified by high performance liquid chromatography to obtain a product preparation liquid, and the preparation liquid was lyophilized to obtain the solid compound 59 (43 mg); LC-MS: [M+H]⁺=1079.3.

### Example 66

### Synthesis of Compound 60

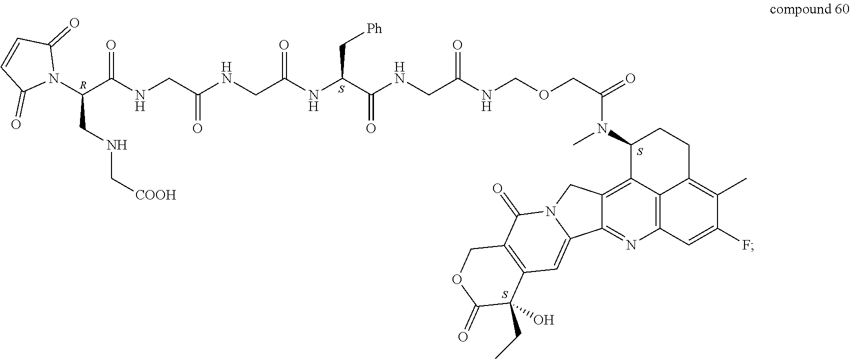

Referring to the synthetic route of Example 65, compound 60 (40 mg) was obtained; LC-MS: [M+H]⁺=1079.3.

### Example 67

### Synthesis of Compound 61

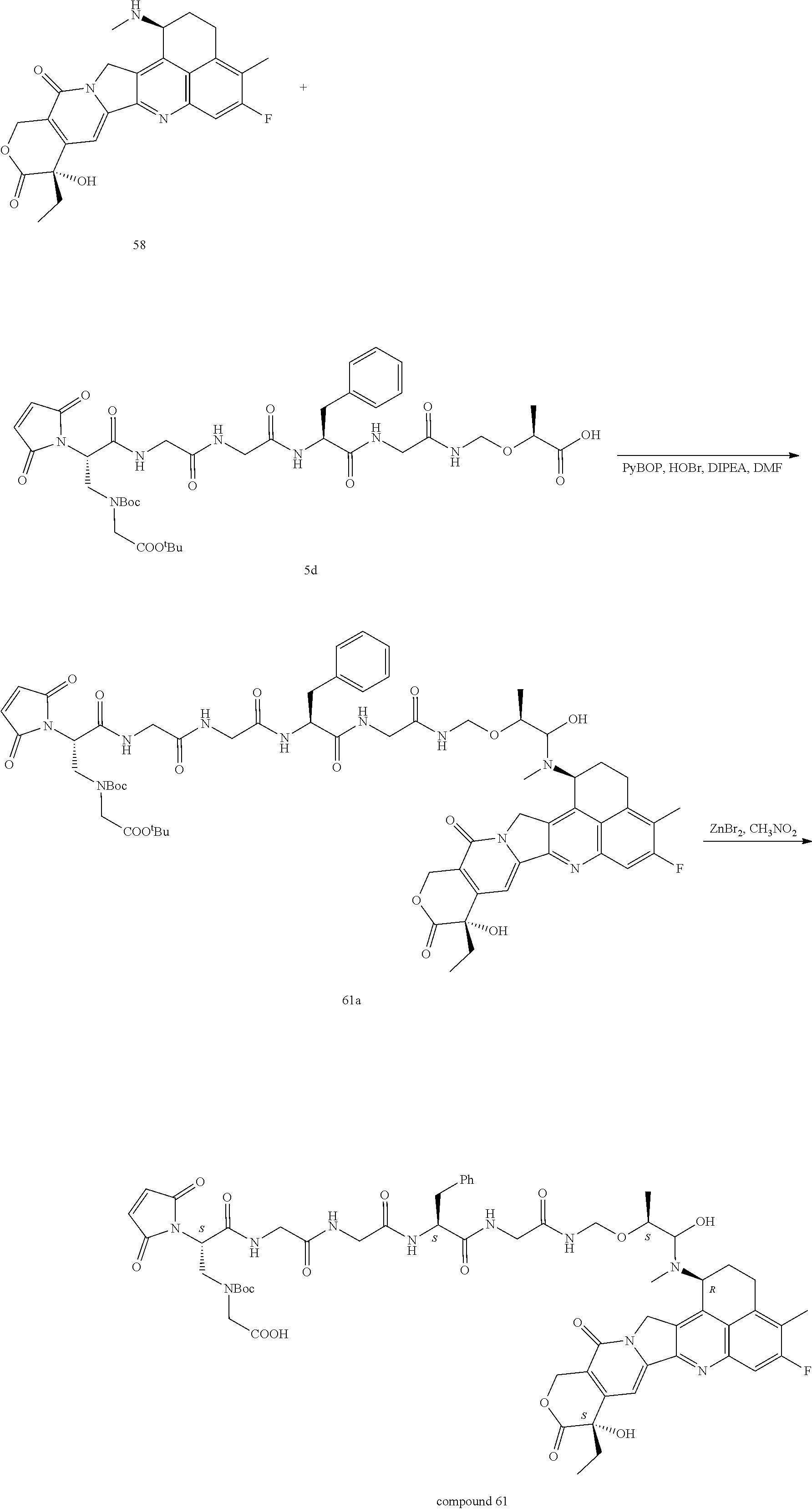

### Step 1: Compound 61a

Add 5d (1.66 g, 2.02 mmol, 1.0 eq), 58 (0.91 g, 2.02 mmol, 1.0 eq), PyBOP (1.58 g, 3.03 mmol, 1.5 eq), HOBt (0.41 g, 3.03 mmol, 1.5 eq), and DMF (40 mL) in a 100 mL single-necked flask, add DIPEA (0.84 mL, 1.5 eq) in an ice-water bath, and raise the temperature to room temperature to react for 2 h (monitored by HPLC). The reaction solution was purified directly by preparative LC, the preparation liquid was concentrated at 35° C. in a water bath under reduced pressure with a water pump to remove acetonitrile, and lyophilized to obtain compound 61a (1.21 g); LC-MS: [M+H]⁺=1249.4.

### Step 2: Compound 61

Add compound 61a (1.0 g, 0.8 mmol, 1.0 eq) and 35 mL nitromethane into a 100 mL single-necked flask, and after dissolving, add zin bromide (3.64 g, 16 mmol, 20.0 eq), react in an oil bath at 40° C. (preheated to stabilize) for 30 min, and concentrate at 45° C. in a water bath under reduced pressure with a water pump to remove nitromethane, to obtain a yellow residue solid (monitored by HPLC). After acid preparation, a preparation liquid was obtained. The preparation liquid was concentrated at 35° C. in a water bath under reduced pressure with a water pump to remove acetonitrile by spinning, and freeze-dried to obtain compound 61 (786 mg). LC-MS: [M+H]⁺=1093.6.

### Example 68

### Synthesis of Compound 62

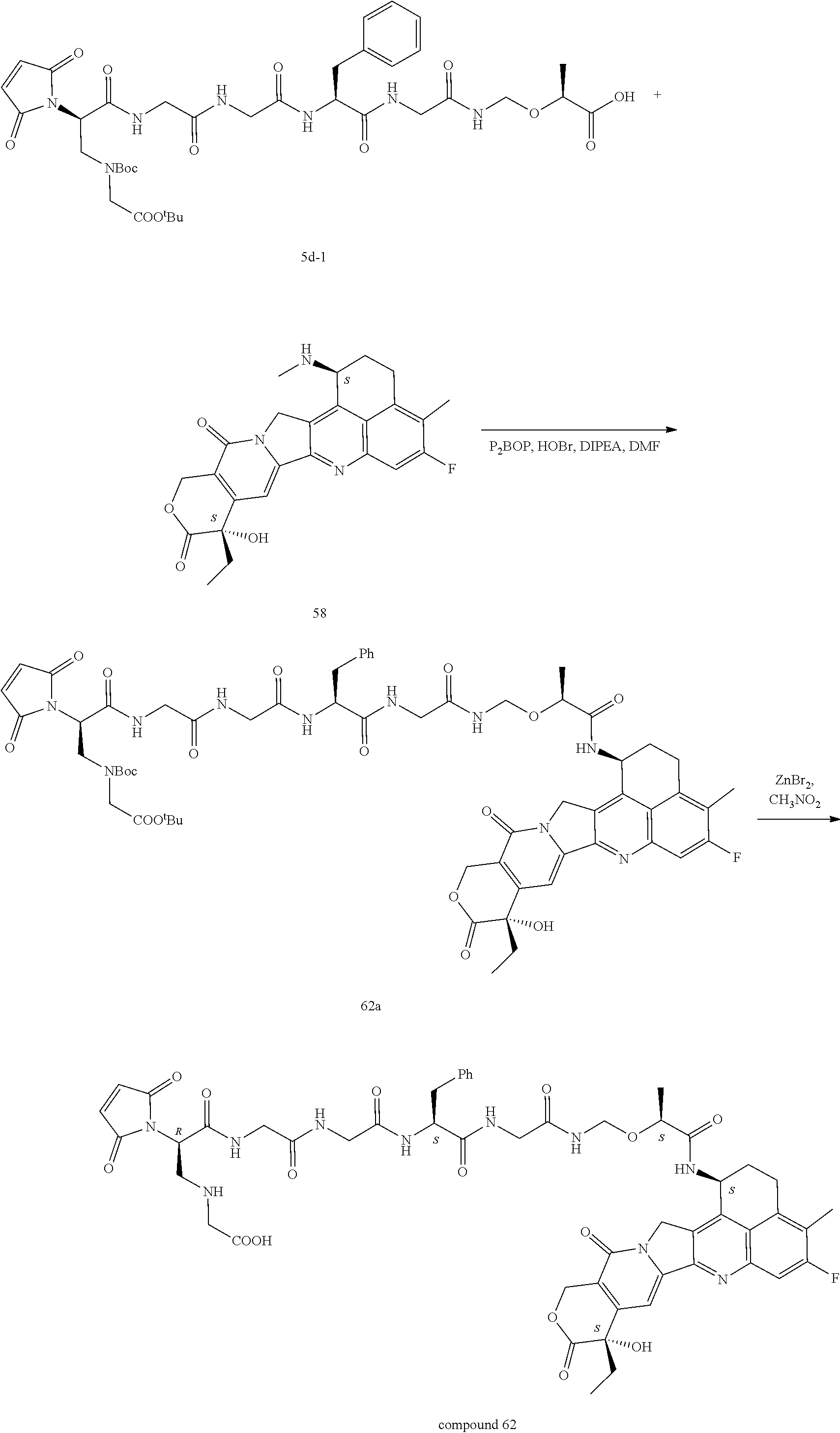

### Step 1: Compound 62a

Add compound 5d-1 (200 mg, 0.24 mmol, 1.0 eq), 58 (110.3 mg, 0.24 mmol, 1.0 eq), PyBOP (187 mg, 0.36 mmol, 1.2 eq), HOBt (48 mg, 0.36 mmol, 1.2 eq) and DMF (6 mL) to a 25 mL single-necked flask, lower to 0-5° C. in an ice-water bath, and add DIPEA (62 mg, 0.48 mmol, 2.0 eq). Then raise to 20±5° C. to react for 2 h, and use HIPLC to monitor for the end of the reaction. The reaction solution was directly purified by HIPLC preparation, and the product preparation liquid was collected and freeze-dried to obtain compound 62a (120.9 mg); LC-MS: [M+H]⁺=1249.4.

### Step 2: Compound 62

Add compound 62a (100 mg, 0.081 mmol, 1.0 eq), ZnBr₂ (364 mg, 1.62 mmol, 20.0 eq) and CH₃NO₂ (10 mL) sequentially into a 25 mL single-necked flask. After the addition is complete, raise the temperature to 40° C. and react for 0.5 h. The reaction was stopped, and the reaction solution was directly spin-dried under reduced pressure at 45° C. to obtain a yellow solid, which was sampled to monitor the reaction by HPLC. The spin-dried solid was directly purified by HPLC preparation, and the product preparation liquid was collected and lyophilized to obtain compound 62 (61 mg); LC-MS: [M+H]⁺=1093.4.

### Example 69

### Preparation of Compound 63

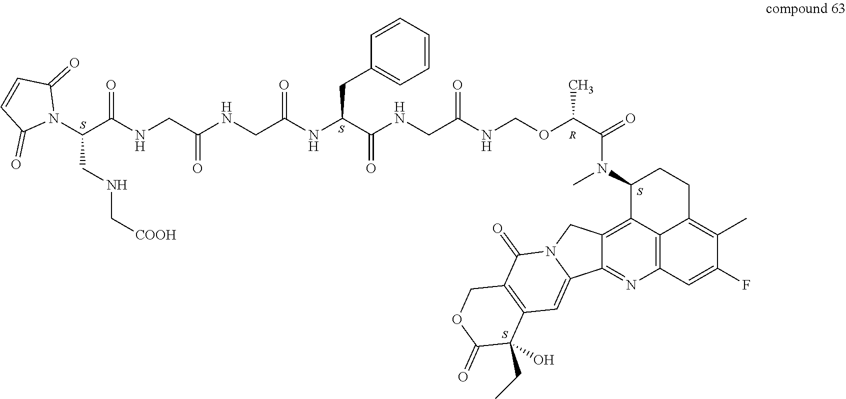

Referring to the route of Example 67, compound 63 (60 mg) was obtained; LC-MS: [M+H]⁺=1093.4.

### Example 70

### Preparation of Compound 64

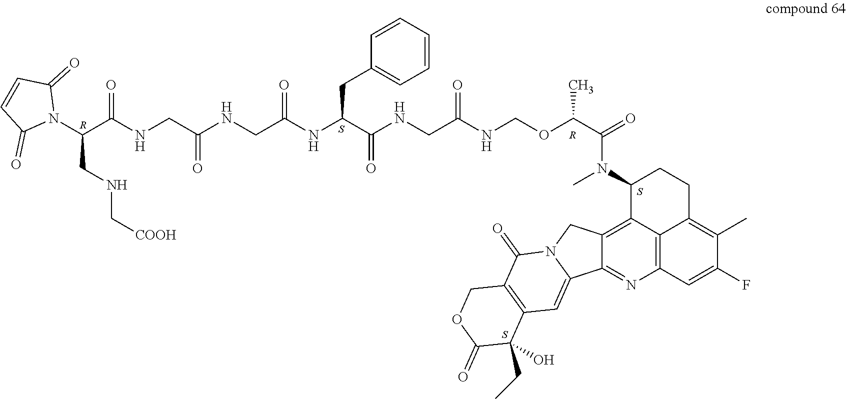

Referring to the synthetic route of Example 68, compound 64 (65 mg) was obtained; LC-MS: [M+H]⁺=1093.4.

### Example 71

### Preparation of Compounds 65A and 65B

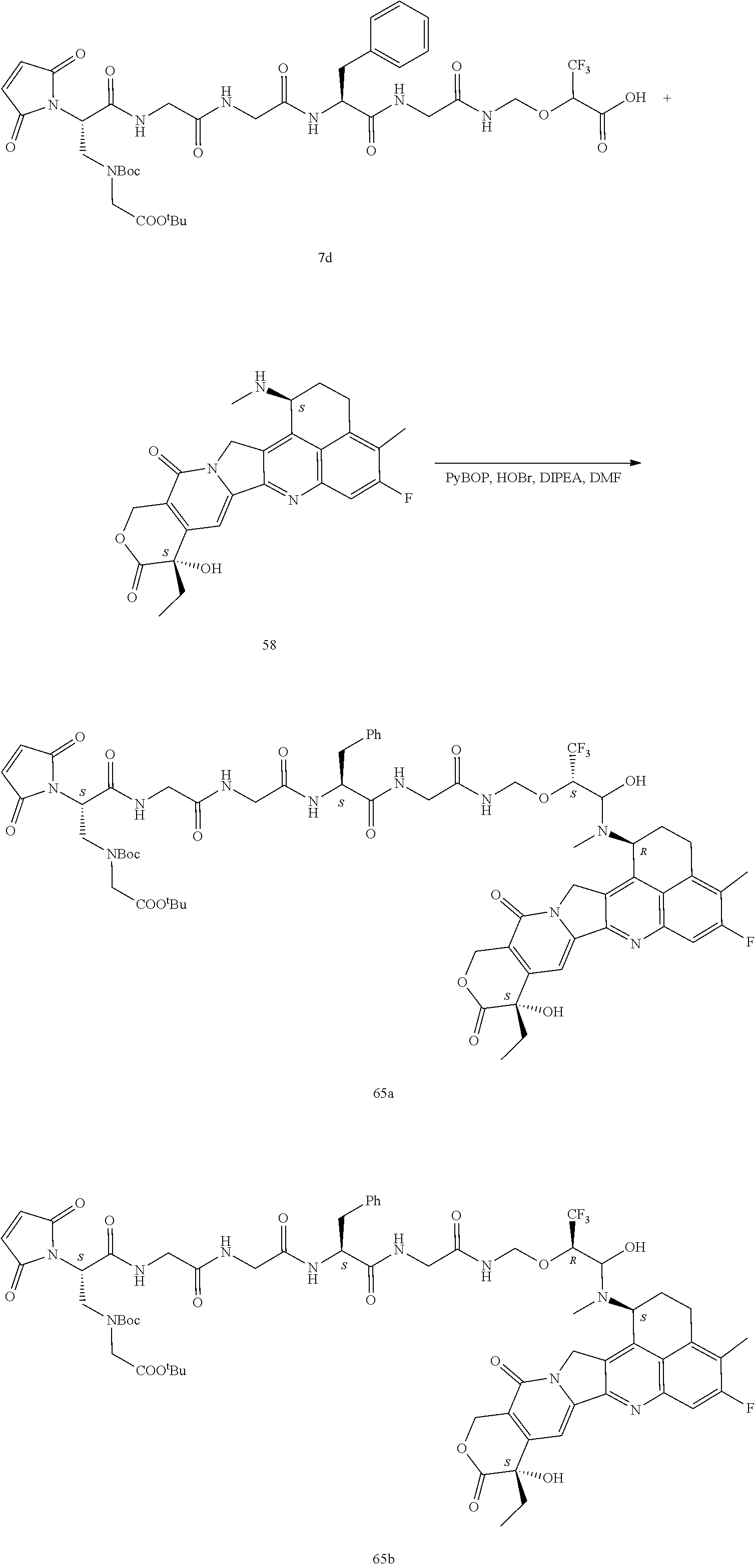

### Step 1: Compounds 65a and 65b

Add 7d (500 mg, 0.57 mmol), 58 (256.8 mg, 0.57 mmol), PyBOP (448 mg, 0.86 mmol), HOBt (116 mg, 0.86 mmol) and 15 mL DMF into a 50 mL one-necked flask, add DIPEA (378 uL, 2.29 mmol) in an ice-water bath, raise to room temperature and react for 2 h. After the completion of the reaction as detected by HIPLC, the reaction solution was purified by HPLC to obtain the preparation liquids of compounds 65a and 65b. The preparation liquids were lyophilized separately to obtain 155 mg of compound 65a, LC-MS: [M+H]⁺=1303.4, and 158 mg of compound 65b, LC-MS: [M+H]⁺=1303.6.

### Step 2: Compound 65A

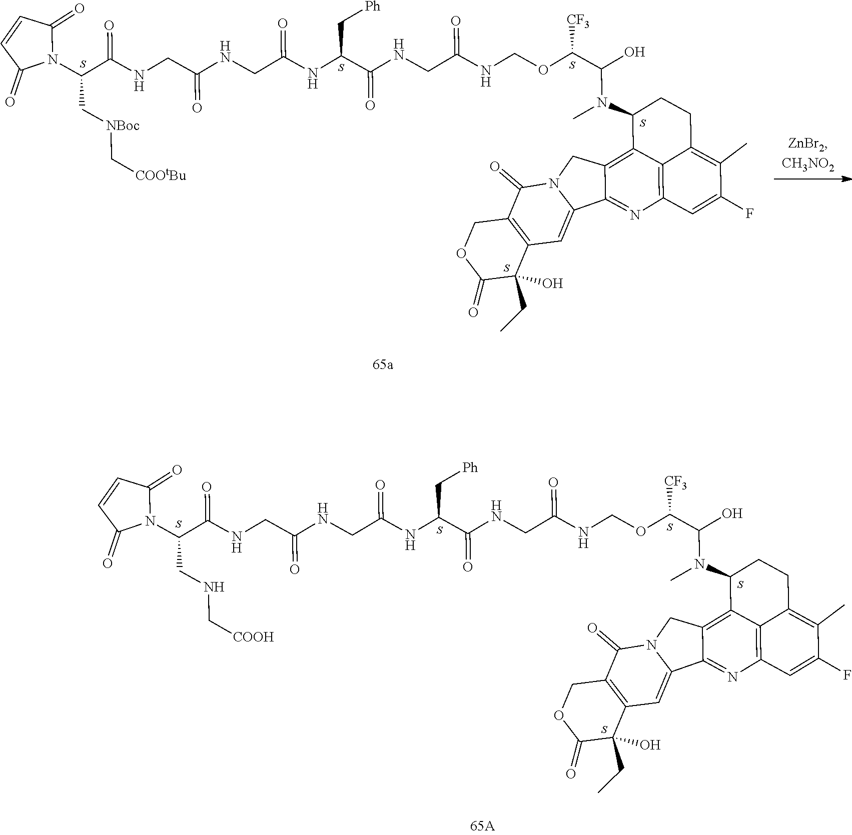

Add 50a (100 mg, 0.077 mmol), zinc bromide (349 mg, 1.55 mmol) and 5 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HPLC to obtain a preparation liquid, which was lyophilized to obtain 49 mg of solid.

### Step 3: Compound 65B

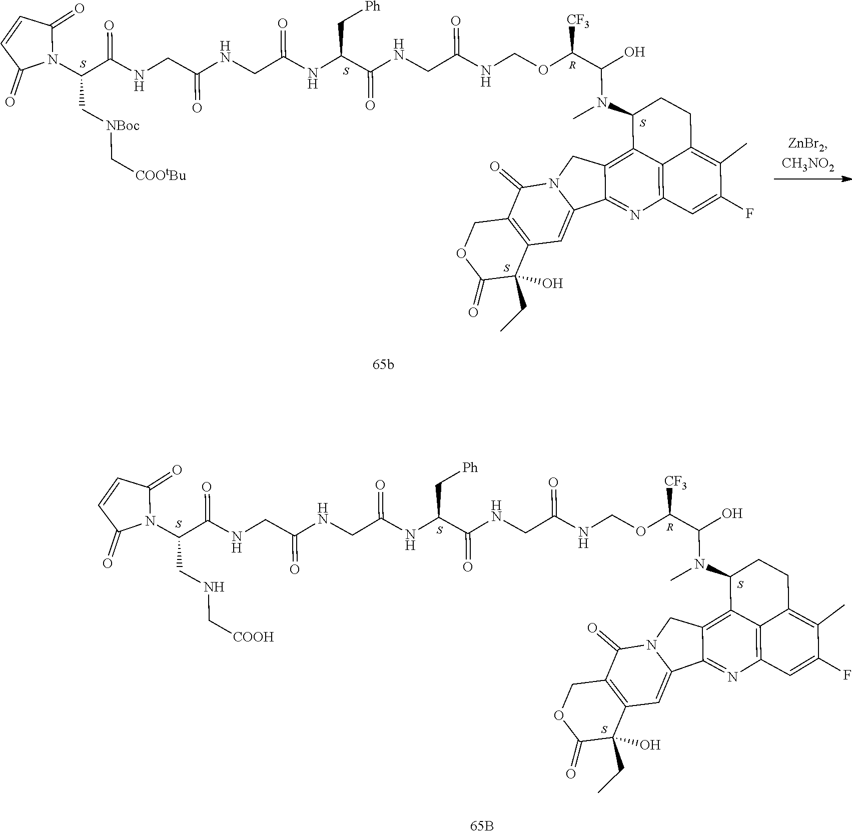

Add 65b (100 mg, 0.077 mmol), zinc bromide (349 mg, 1.55 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a preparation liquid, which was lyophilized to obtain 47 mg of solid.

### Example 72

### Synthesis of Compounds 66A and 66B

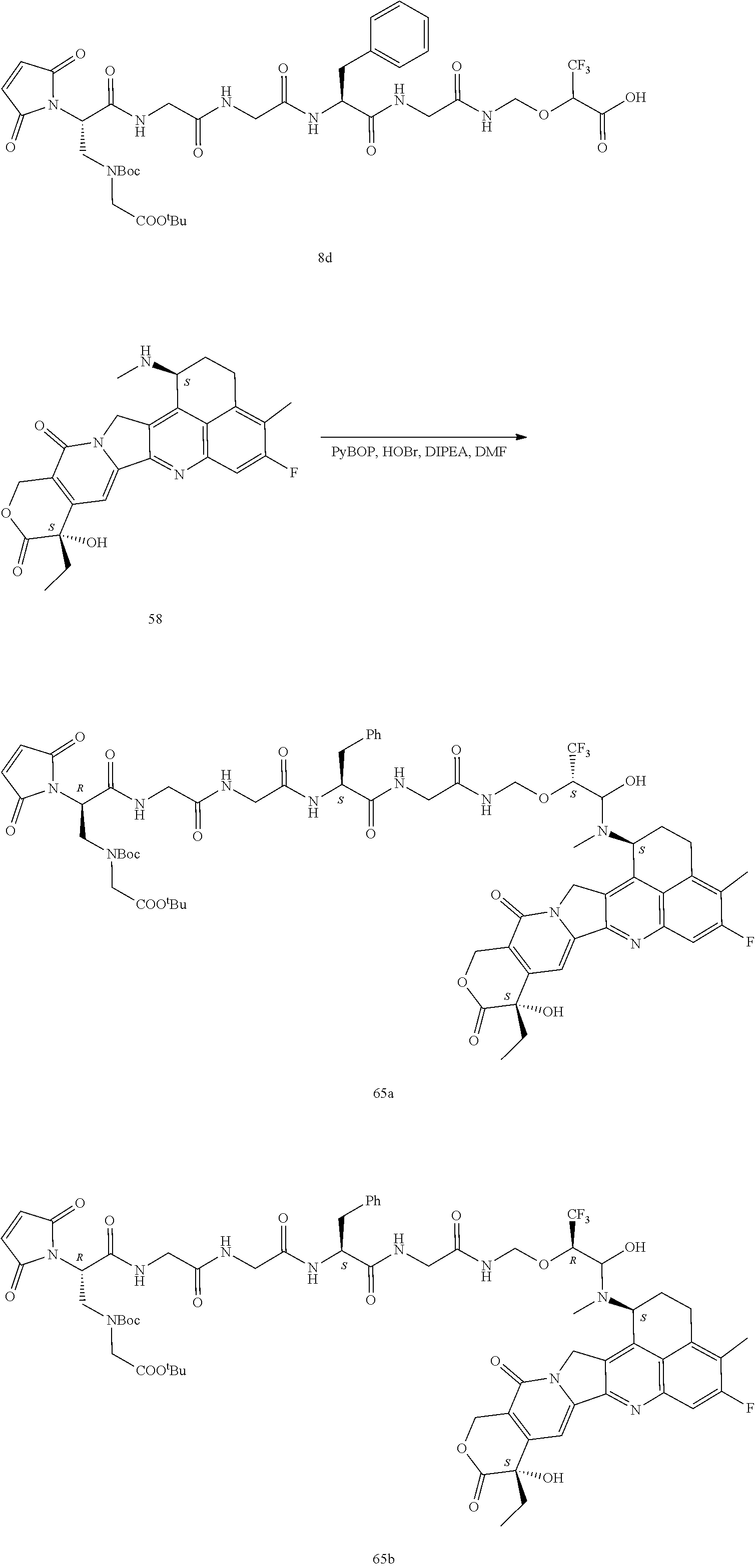

### Step 1: Compound 66a and Compound 66b

Add 8d (500 mg, 0.57 mmol), 58 (256.8 mg, 0.57 mmol), PyBOP (448 mg, 0.86 mmol), HOBt (116 mg, 0.86 mmol) and 15 mL DMF into a 50 mL one-necked flask, add DIPEA (378 uL, 2.29 mmol) in an ice-water bath, raise to room temperature and react for 2 h. After the completion of the reaction as detected by HPLC, the reaction solution was purified by HIPLC to obtain the preparation liquids of compound 66a and compound 66b, and the preparation liquids were separately lyophilized to obtain 160 mg of compound 66a and 160 mg of compound 66b. LC-MS of compound 66a: [M+H]⁺=1303.7; LC-MS of compound 66b: [M+H]⁺=1303.6.

### Step 2: Compound 66A

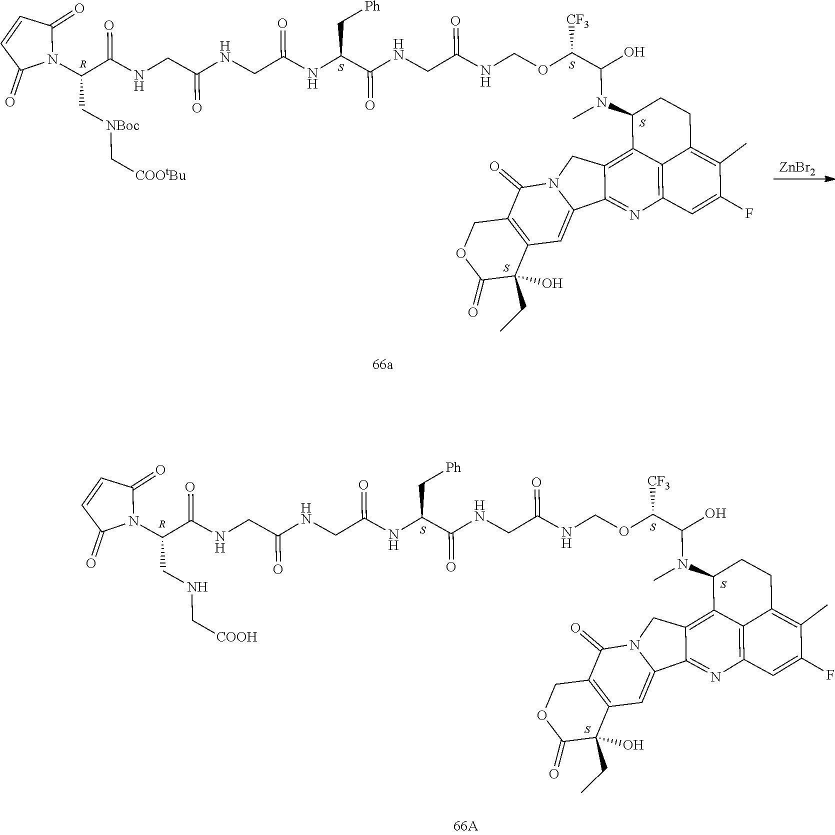

Add compound 66a (100 mg, 0.077 mmol), zinc bromide (349 mg, 1.55 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain 57 mg of solid, LC-MS: [M+H]⁺=1147.5.

### Step 3: Compound 66B

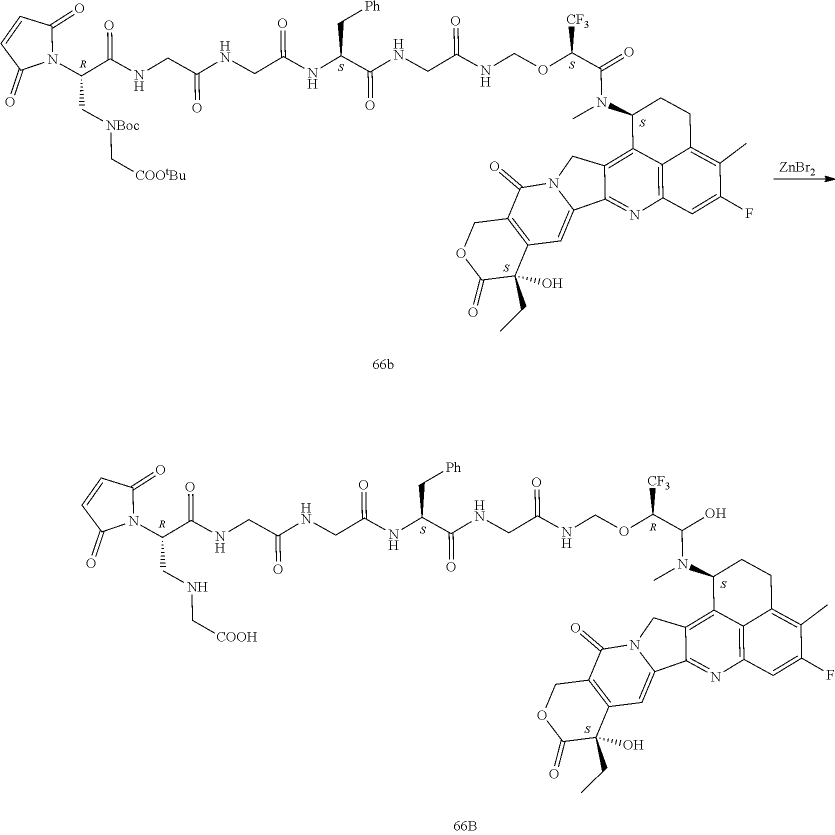

Add compound 66b (100 mg, 0.077 mmol), zinc bromide (349 mg, 1.55 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain 57 mg of solid, LC-MS: [M+H]⁺=1147.5.

### Example 73

### Synthesis of Compound 67A

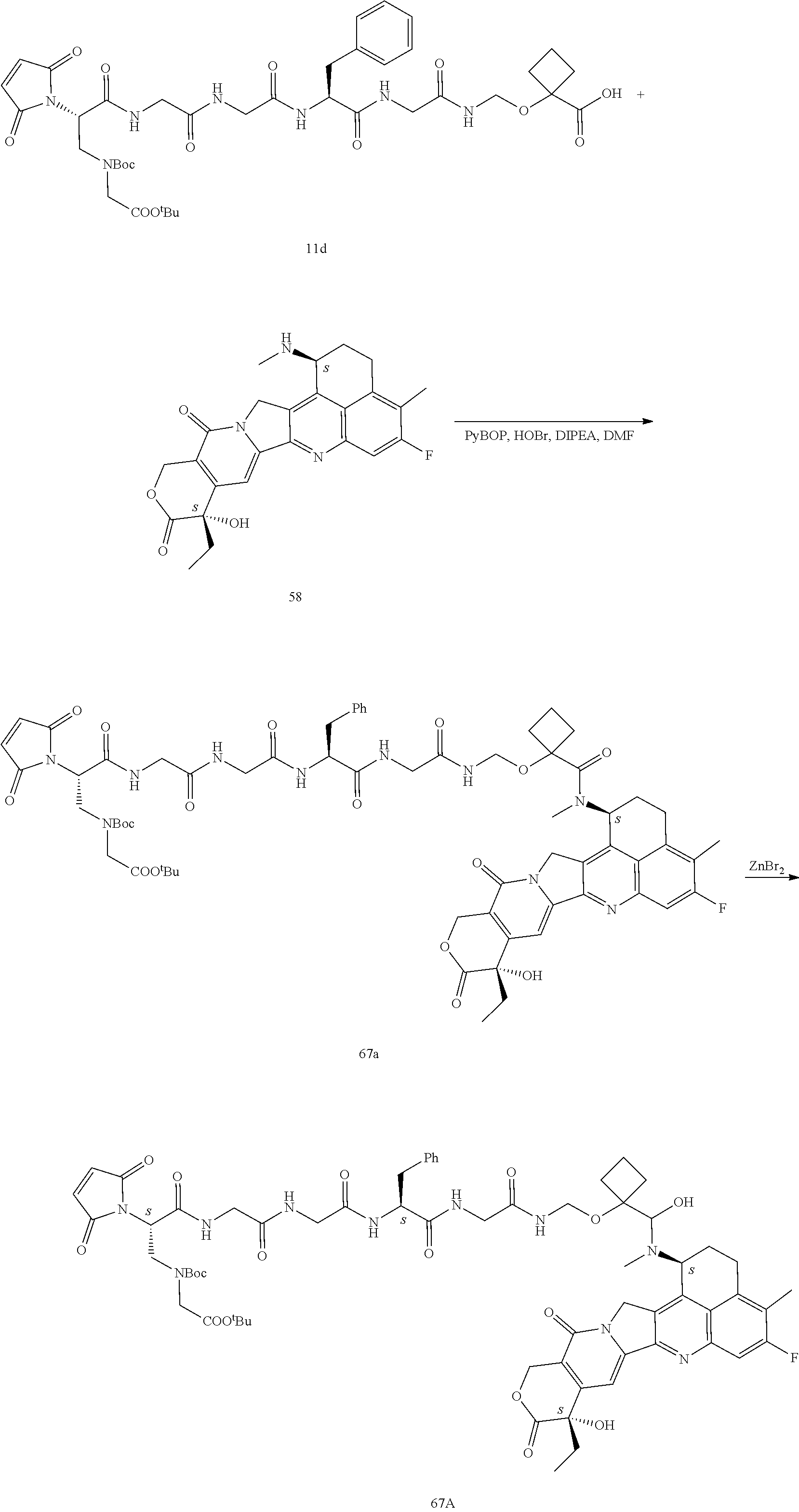

### Step 1: Compound 67a

Add 11d (800 mg, 0.96 mmol), 58 (432.5 mg, 0.96 mmol), PyBOP (500 mg, 0.96 mmol), HOBt (208 mg, 0.96 mmol) and 30 mL DMF into a 50 mL single-necked flask, add DIPEA (660 uL, 4.0 mmol) in an ice-water bath, raise to room temperature and react for 4 h. After the completion of the reaction as detected by HPLC, the reaction solution was purified by HPLC to obtain a preparation liquid of compound 67a, which was lyophilized to obtain 67a (402 mg); LC-MS: [M+H]⁺=1275.4.

### Step 2: Compound 67A

Add 67a (100 mg, 0.78 mmol), zinc bromide (356 mg, 1.57 mmol) and 10 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain solid compound 67A (47 mg); LC-MS: [M+H]⁺=1119.5.

### Example 74

### Synthesis of Compound 67B

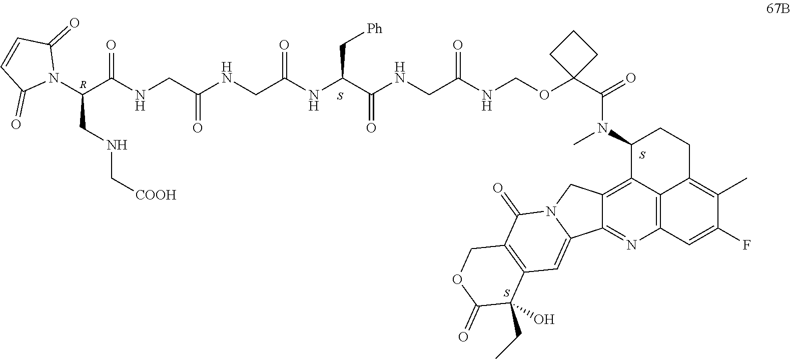

Referring to the synthetic route of Example 73, compound 67B (50 mg) was obtained. LC-MS: [M+H]⁺ 1119.4.

### Example 75

### Synthesis of Compound 68A

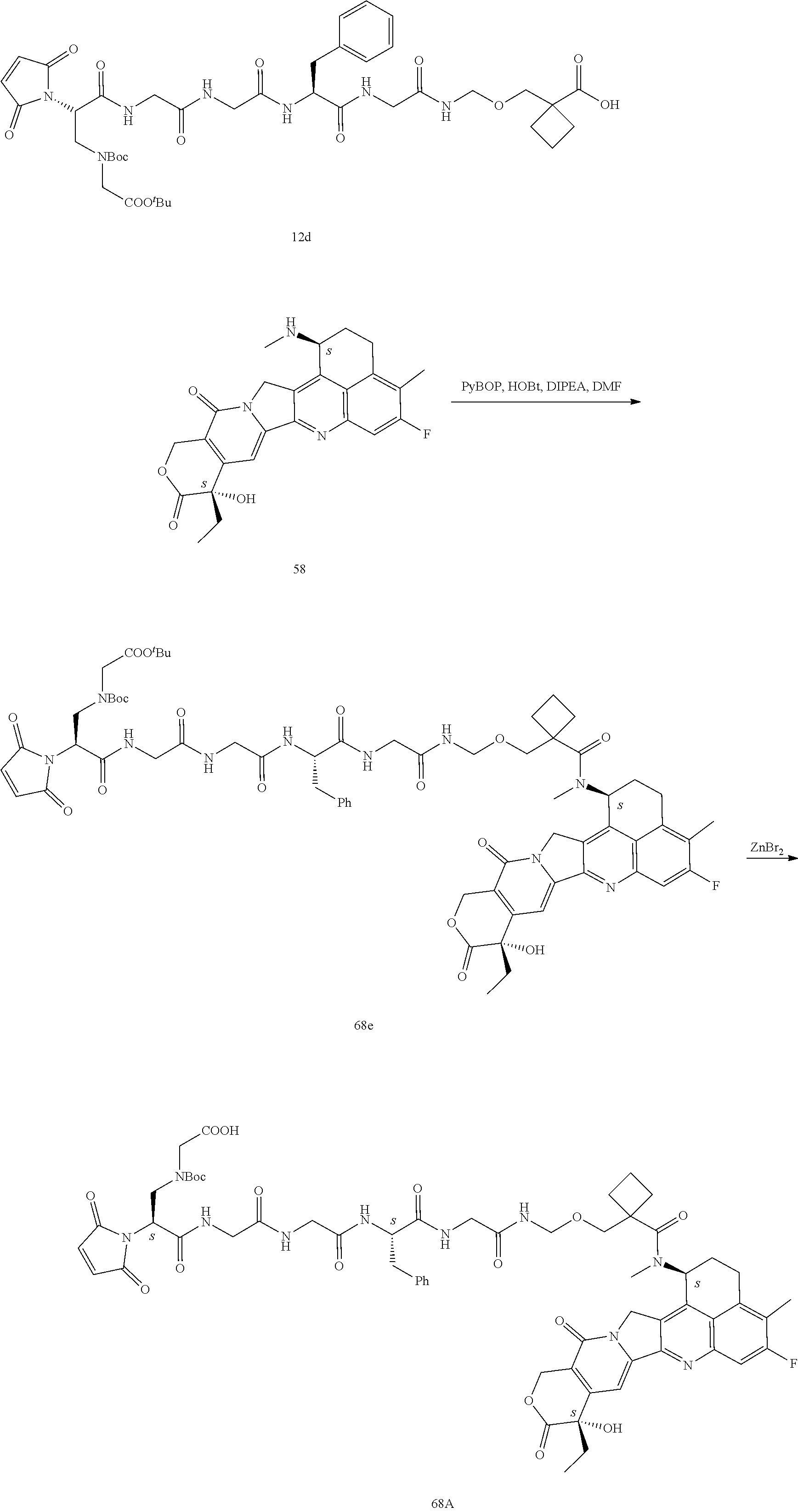

### Step 1: Compound 68a

Add 12d (400 mg, 0.47 mmol), 58 (211.7 mg, 0.47 mmol), PyBOP (250 mg, 0.47 mmol), HOBt (101 mg, 0.47 mmol) and 15 mL DMF into a 50 mL single-necked flask, add DIPEA (330 uL, 2.0 mmol) in an ice-water bath, and raise to room temperature and react for 3 h. After the completion of the reaction as detected by HPLC, the reaction solution was purified by HIPLC to obtain a preparation liquid of compound 68a, which was lyophilized to obtain 68a (177 mg); LC-MS: [M+H]⁺=1289.4.

### Step 2: Compound 68A

Add 68a (100 mg, 0.08 mmol), zine bromide (360 mg, 1.6 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HIPLC to obtain a product preparation liquid, which was lyophilized to obtain solid compound 68A (45 mg); LC-MS: [M+H]⁺=1133.4.

### Example 76

### Synthesis of Compound 68B

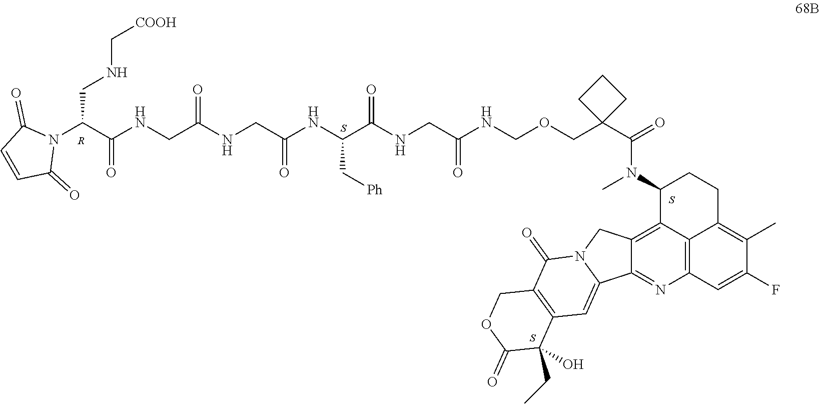

Referring to the synthetic route of Example 75, compound 68B (50 mg) was obtained; LC-MS: [M+H]⁺=1133.4.

### Example 77

### Synthesis of Compounds 69A and 68B

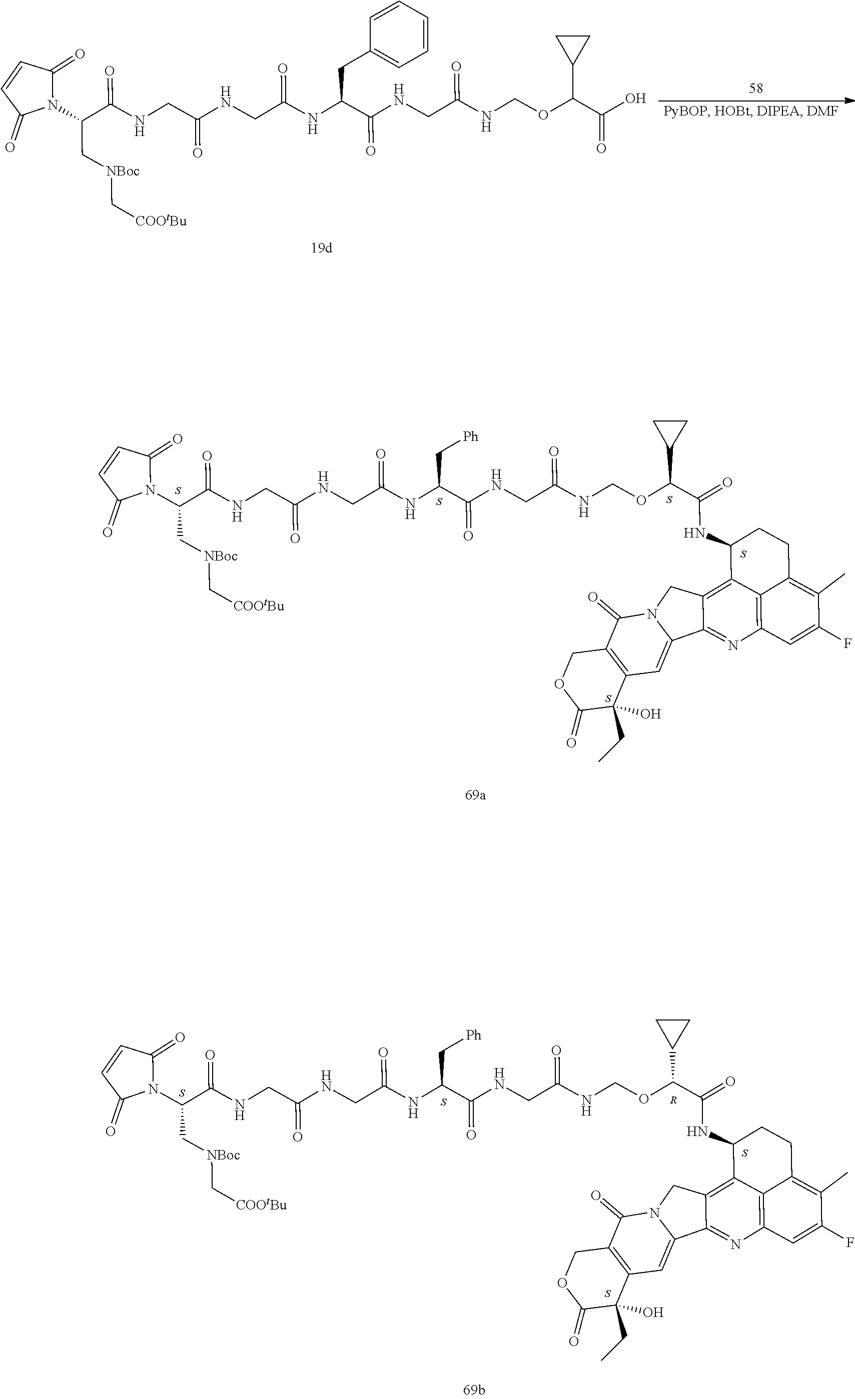

### Step 1: Compounds 69a and 69b

Add 19d (500 mg, 0.59 mmol), 58 (266 mg, 0.59 mmol), PyBOP (339 mg, 0.65 mmol), HOBt (88 mg, 0.86 mmol) and 10 mL DMF into a 50 mL single-necked flask, add DIPEA (292 uL, 1.77 mmol) in an ice-water bath, raise to room temperature and react for 2 h. After the completion of the reaction as detected by HIPLC, the reaction solution was purified by HIPLC to obtain the preparation liquids of compounds 69a and 69b. The preparation solutions were freeze-dried separately to obtain 109 mg of compound 69a, LC-MS: [M+H]⁺=1275.5, and 111 mg of compound 69b, LC-MS: [M+H]⁺=1275.7.

### Step 2: Compound 69A

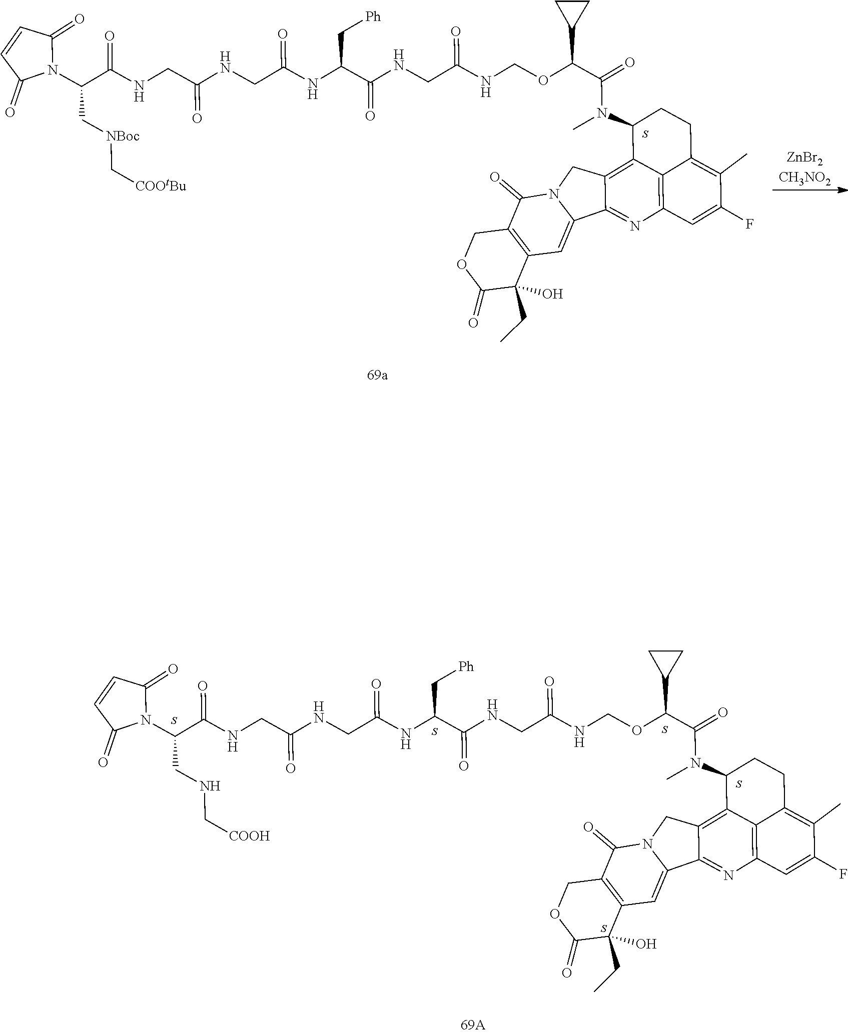

Add 69a (100 mg, 0.078 mmol), zinc bromide (352 mg, 1.56 mmol) and 5 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HPLC to obtain a product preparation liquid, which was lyophilized to obtain 53 mg of solid; LC-MS: [M+H]⁺=1119.4.

### Step 3: Compound 69B

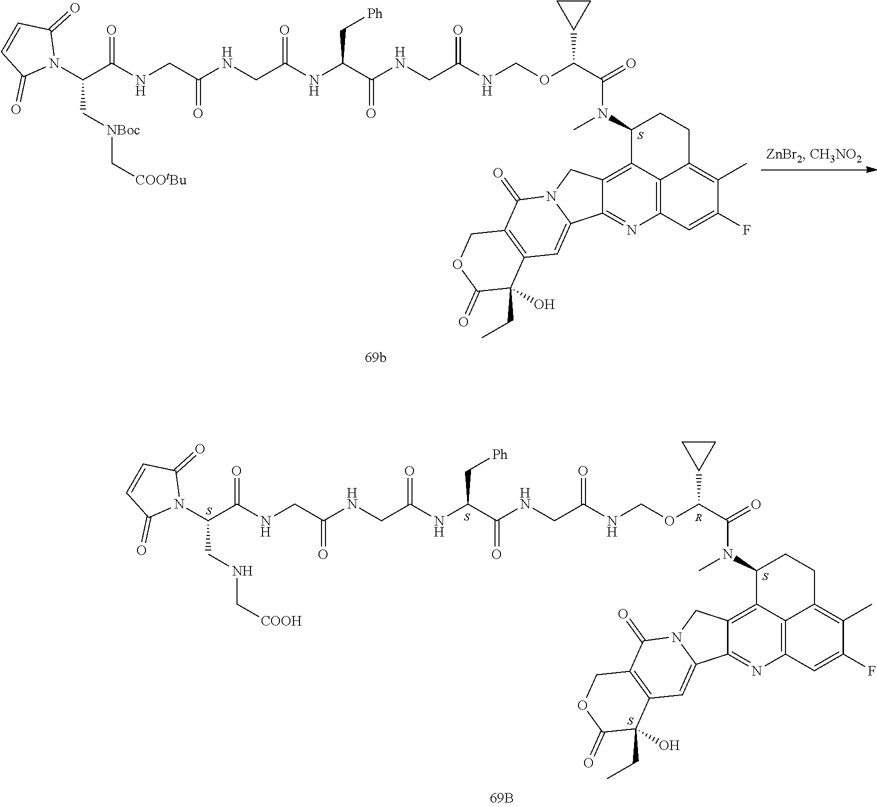

Add 69b (100 mg, 0.078 mmol), zinc bromide (352 mg, 1.56 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HIPLC to obtain a product preparation liquid, which was lyophilized to obtain 54 mg of solid; LC-MS: [M+H]⁺=1119.4.

### Example 78

### Synthesis of Compounds 70A and 7B

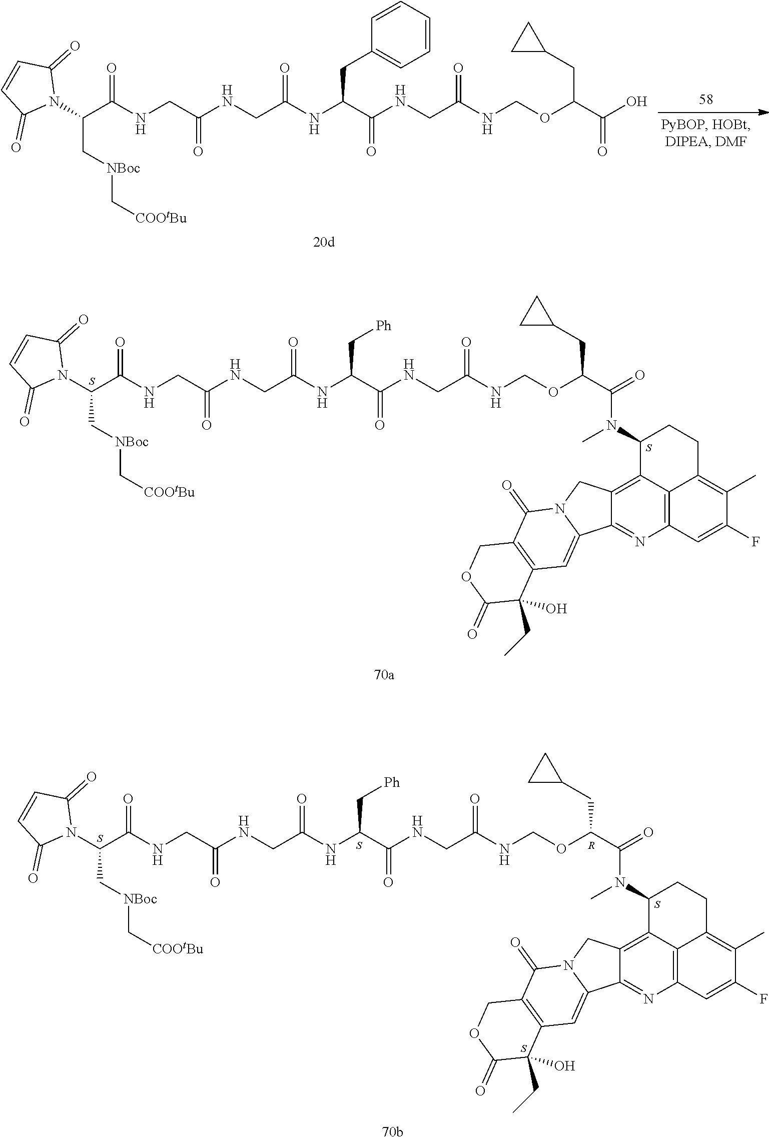

### Step 1: Compounds 70a and 70b

Add 20d (400 mg, 0.47 mmol), 58 (211.7 mg, 0.47 mmol), PyBOP (223 mg, 0.56 mmol), HOBt (83 mg, 0.56 mmol) and 10 mL DMF into a 50 mL single-necked flask, add DIPEA (248 uL, 1.5 mmol) in an ice-water bath, raise to room temperature and react for 2 h. After the completion of the reaction as detected by HPLC, the reaction solution was purified by HIPLC to obtain the preparation liquids of compounds 70a and 70b. The preparation liquids were freeze-dried separately to obtain 106 mg of compound 70a, LC-MS: [M+H]⁺=1289.5, and 101 mg of compound 70b, LC-MS: [M+H]⁺=1289.4.

### Step 2: Compound 70A

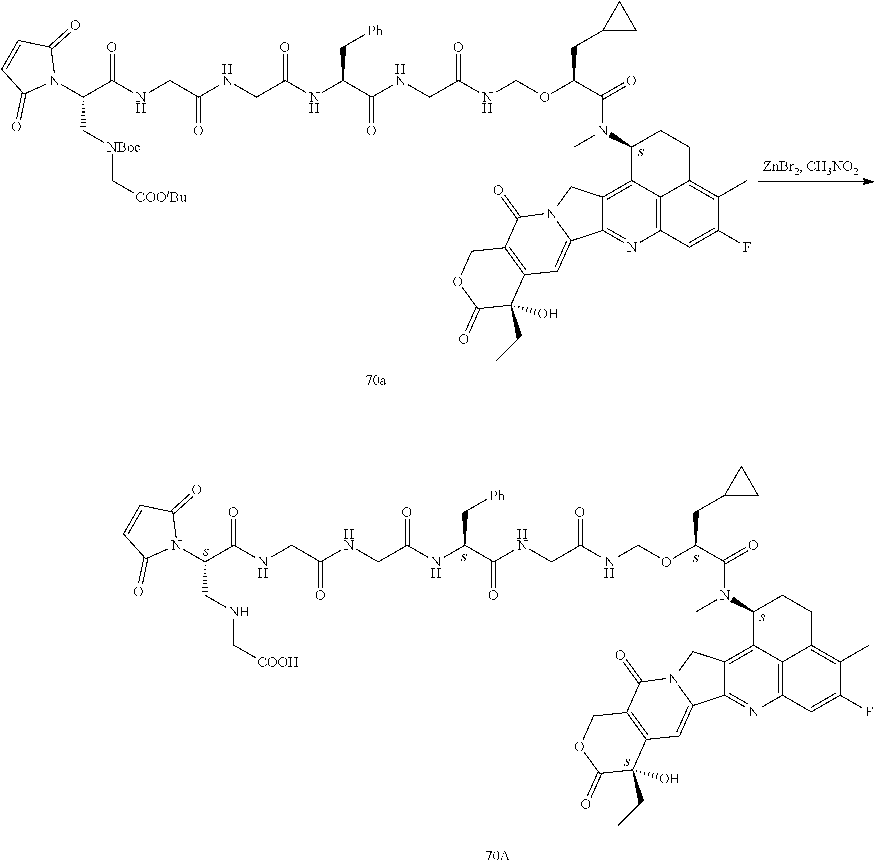

Add 70a (100 mg, 0.078 mmol), zinc bromide (352 mg, 1.57 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain 39 mg of solid; LC-MS: [M+H]⁺=1133.4.

### Step 3: Compound 70B

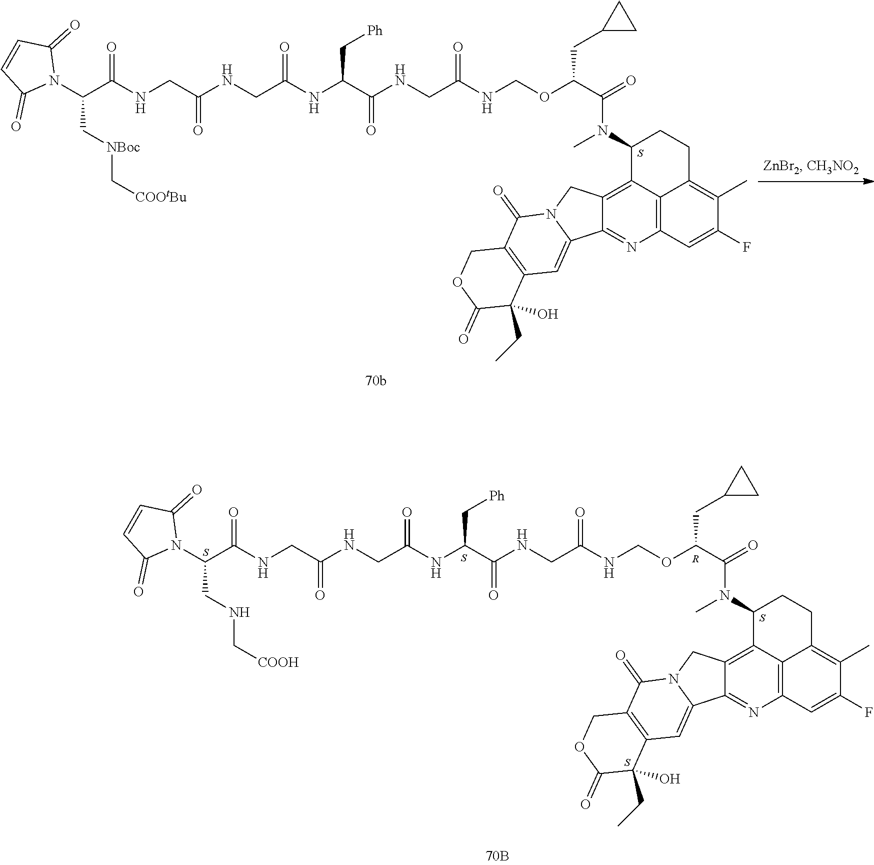

Add 70b (100 mg, 0.078 mmol), zinc bromide (352 mg, 1.56 mmol) and 5 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain 35 mg of solid; LC-MS: [M+H]⁺=1133.4.

### Example 79

### Synthesis of Compound 71

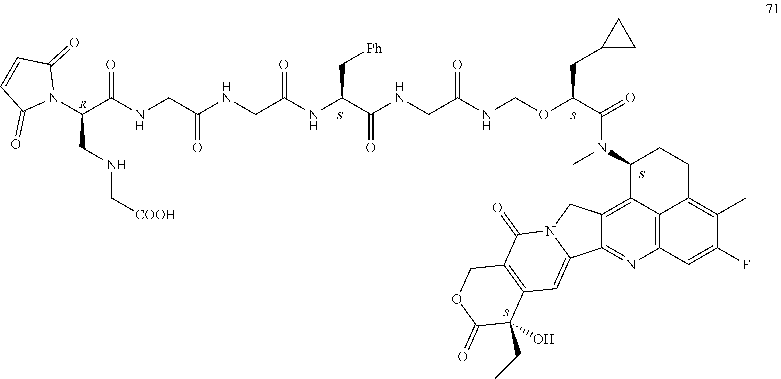

Referring to the synthetic route of Example 78, compound 71 (30 mg) was obtained; LC-MS: [M+H]⁺=1133.3.

### Example 80

### Synthesis of Compound 72

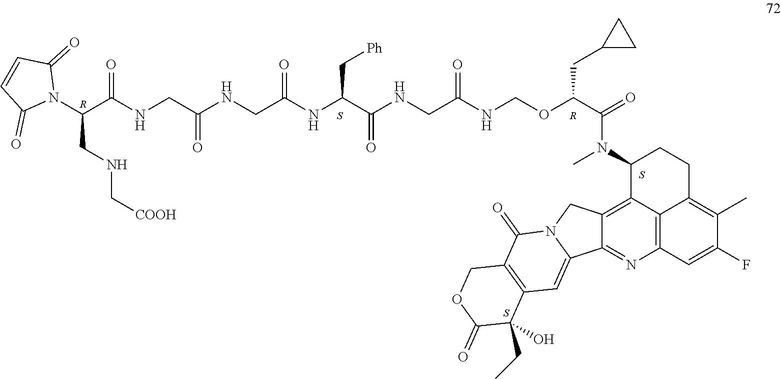

Referring to the synthetic route of Example 78, compound 72 (33 mg) was obtained; LC-MS: [M+H]⁺=1133.4.

### Example 81

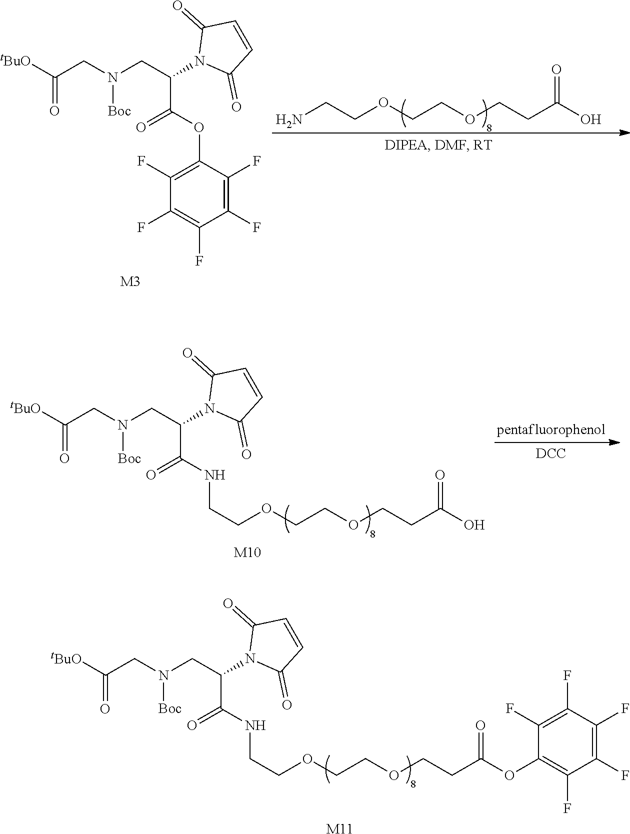

### Synthesis of Compound M11

In a 100 mL single-necked flask, add compound M3 (11.0 g, 19.5 mmol, 1.0 eq), DIPEA (2.8 g, 21.4 mmol, 1.1 eq), 27-amino-4,7,10,13,16,19,22,25-octaoxaheptacosanoic acid (9.7 g, 20.5 mmol, 1.05 eq) and DMF (60 mL), and react at room temperature (monitored by TLC) for 20 minutes. The reaction solution was purified directly by preparative LC, and the preparation liquid was concentrated at 35° C. in a water bath under reduced pressure with a water pump to remove acetonitrile, and lyophilized to obtain compound M10 (13.2 g) in a yield of 78%; LC-MS: [M+H]⁺=866.5.

Add compound M10 (13.0 g, 15 mmol, 1.0 eq), pentafluorophenol (3 g, 16.5 mmol, 1.1 eq), DCC (3.4 g, 16.5 mmol, 1.1 eq) and THF (30 mL) in a 100 mL single-necked flask, react at room temperature for 30 min (monitored by TLC), and filter off the insoluble material. The reaction solution was directly purified by preparative LC, and the preparation liquid was concentrated at 35° C. in a water bath under reduced pressure with a water pump to remove acetonitrile, and lyophilized to obtain compound M11 (14.2 g) in 92% yield; LC-MS: [M+H]⁺=1032.5.

### Example 82

### Synthesis of Compound 73

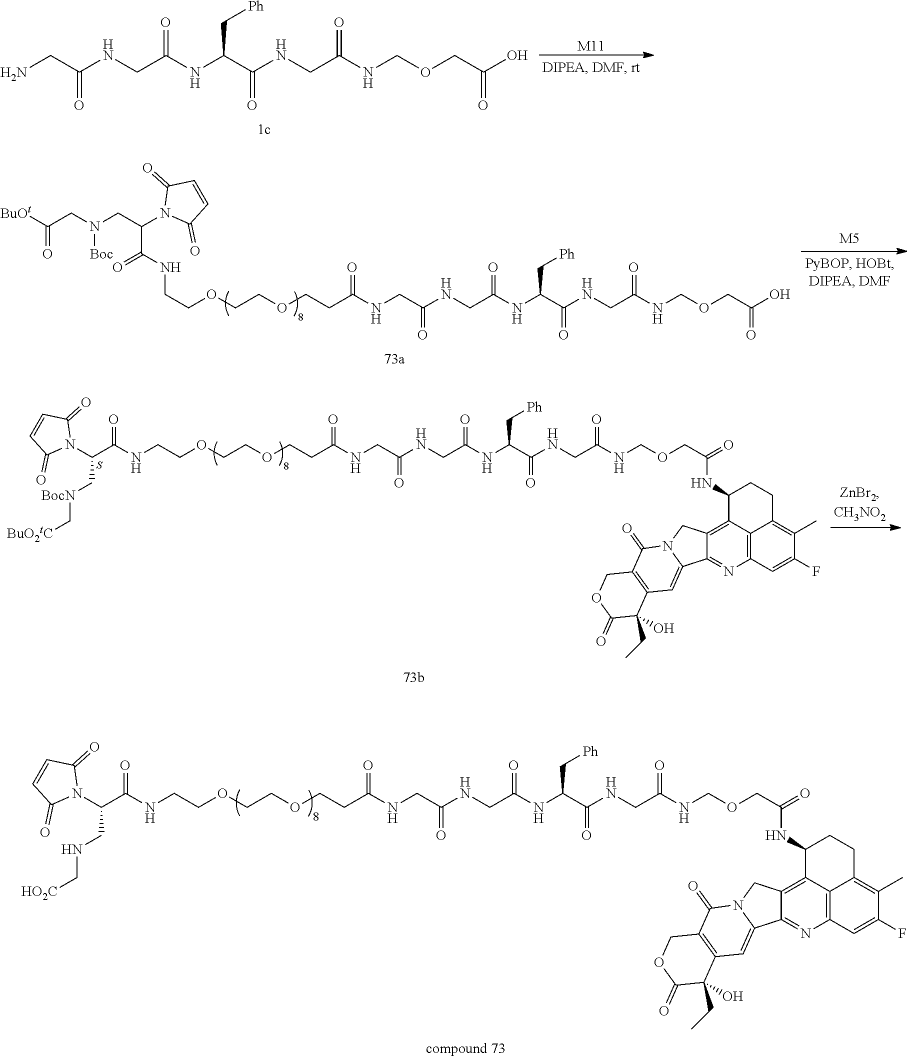

### Step 1: Synthesis of Compound 73a

Add 10 mL of DMF to M11 (1 g, 0.79 mol), cool in an ice-water bath to 0° C., add compound 1c (334 mg, 0.79 mol) and DIPEA (154 mg, 1.19 mol), and maintain the conditions to react for 1 h. TLC monitors for the completion of the reaction. The reaction solution was purified by preparative high-performance liquid chromatography (acetonitrile/purified water system), and the target peak was collected. After removing acetonitrile under reduced pressure, freeze-drying was performed to obtain about 1.2 g of compound 73a, MS m/z: [M+H]⁺=1271.9.

### Step 2: Synthesis of Compound 73b

Add 73a (1.2 g, 0.94 mmol), M5 (500 mg, 0.94 mmol), PyBOP (625 mg, 1.2 mmol), HOBt (162 mg, 1.2 mmol) and 15 mL DMF into a 25 mL single-necked flask, add DIPEA (310 mg, 2.4 mmol) in an ice-water bath, raise to room temperature and react for 2 h. After the completion of the reaction as detected by HPLC, the reaction solution was purified by HPLC to obtain a preparation liquid, which was lyophilized to obtain 73b (709 mg); LC-MS: [M+H]⁺=1720.8.

### Step 3: Synthesis of Compound 73

Add 73b (200 mg, 0.116 mmol), zinc bromide (523 mg, 2.32 mmol) and 10 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain solid compound 73 (88 mg); LC-MS: [M+H]⁺=1532.6.

### Example 83

### Synthesis of Compound 74

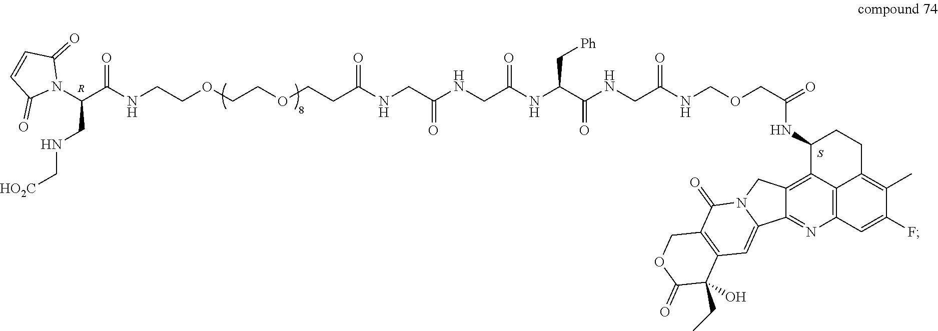

Referring to the synthetic route of Example 82, compound 74 (90 mg) was obtained; LC-MS: [M+H]⁺=1532.6.

### Example 84

### Synthesis of Compound 75

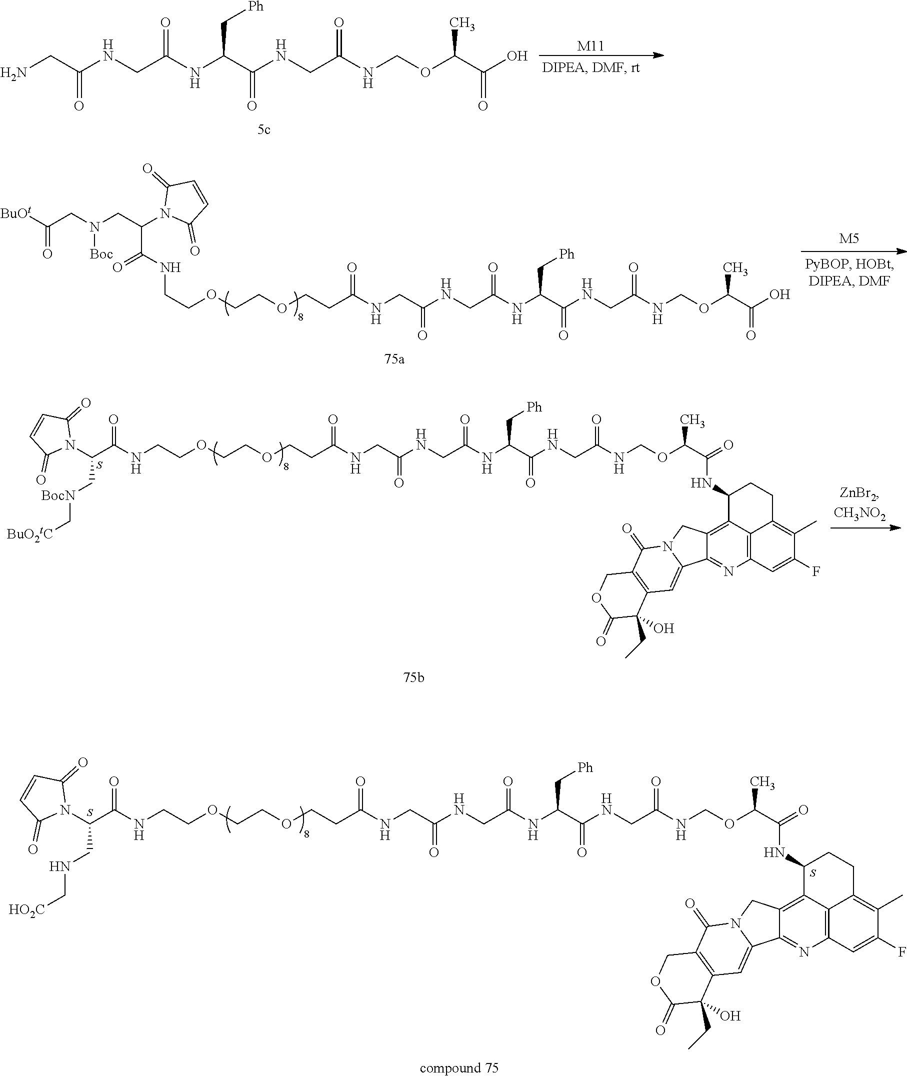

### Step 1: Synthesis of Compound 75a

Add 10 mL of DMF to M11 (1 g, 0.79 mol), cool in an ice-water bath to 0° C., add compound 5c (345 mg, 0.79 mol) and DIPEA (154 mg, 1.19 mol), and maintain the conditions to react for 1 h. TLC monitors for completion of the reaction. The reaction solution was purified by preparative high-performance liquid chromatography (acetonitrile/purified water system), and the target peak was collected. After removing acetonitrile under reduced pressure, lyophilization was performed to obtain 0.9 g of compound 75a, MS m/z: [M+H]⁺=1285.6.

### Step 2: Synthesis of Compound 75b

Add 75a (700 mg, 0.54 mmol), M5 (289 mg, 0.54 mmol), PyBOP (313 mg, 0.6 mmol), HOBt (81 mg, 0.6 mmol) and 10 mL DMF into a 25 mL single-necked flask, add DIPEA (155 mg, 1.2 mmol) in an ice-water bath, raise to room temperature and react for 2 h. After completion of the reaction as detected by HIPLC, the reaction solution was purified by HPLC to obtain a preparation liquid, which was lyophilized to obtain 75b (304 mg); LC-MS: [M+H]⁺=1734.8.

### Step 3: Synthesis of Compound 75

Add 75b (200 mg, 0.116 mmol), zinc bromide (523 mg, 2.32 mmol) and 10 mL nitromethane into a 25 mL single-necked flask and react at 40° C. for 1 h. After the completion of the reaction as detected by HIPLC, the solvent was removed by concentration under reduced pressure to obtain a crude product. The crude product was purified by HPLC to obtain a product preparation liquid, which was lyophilized to obtain solid compound 75 (96 mg); LC-MS: [M+H]⁺=1546.6.

### Example 85

### Synthesis of Compound 76

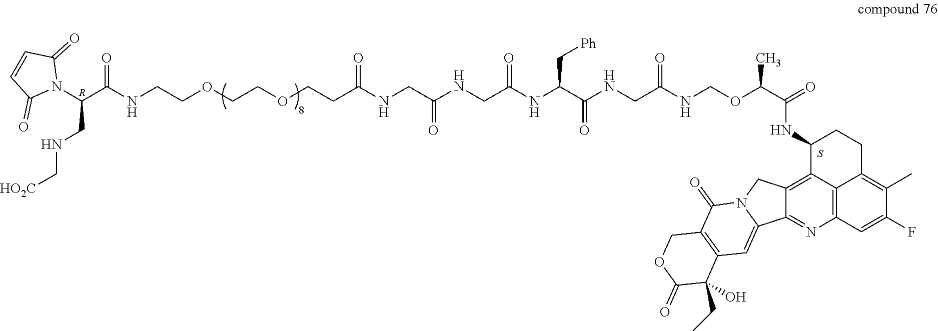

Referring to the synthetic route of Example 84, compound 76 (92 mg) was obtained; LC-MS: [M+H]⁺=1546.5.

### Example 86

### Synthesis of Compound 77

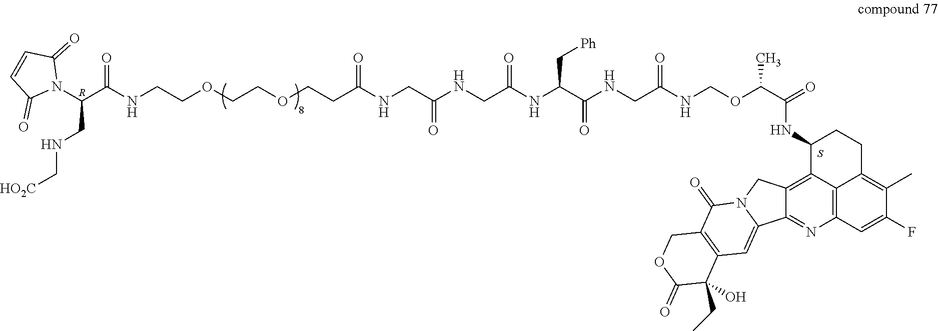

Referring to the synthetic route of Example 84, compound 77 (87 mg) was obtained; LC-MS: [M+H]⁺=1546.5.

### Example 87

### Synthesis of Compound 78

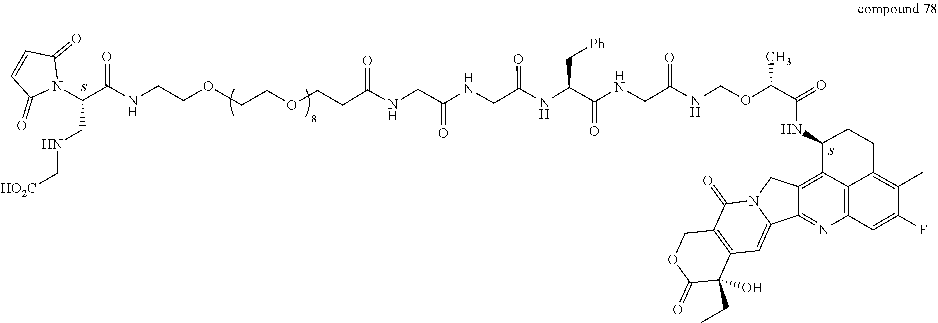

Referring to the synthetic route of Example 84, compound 78 (94 mg) was obtained; LC-MS: [M+H]⁺=1546.7.

### Example 88

### Synthesis of Compounds 79 and 80

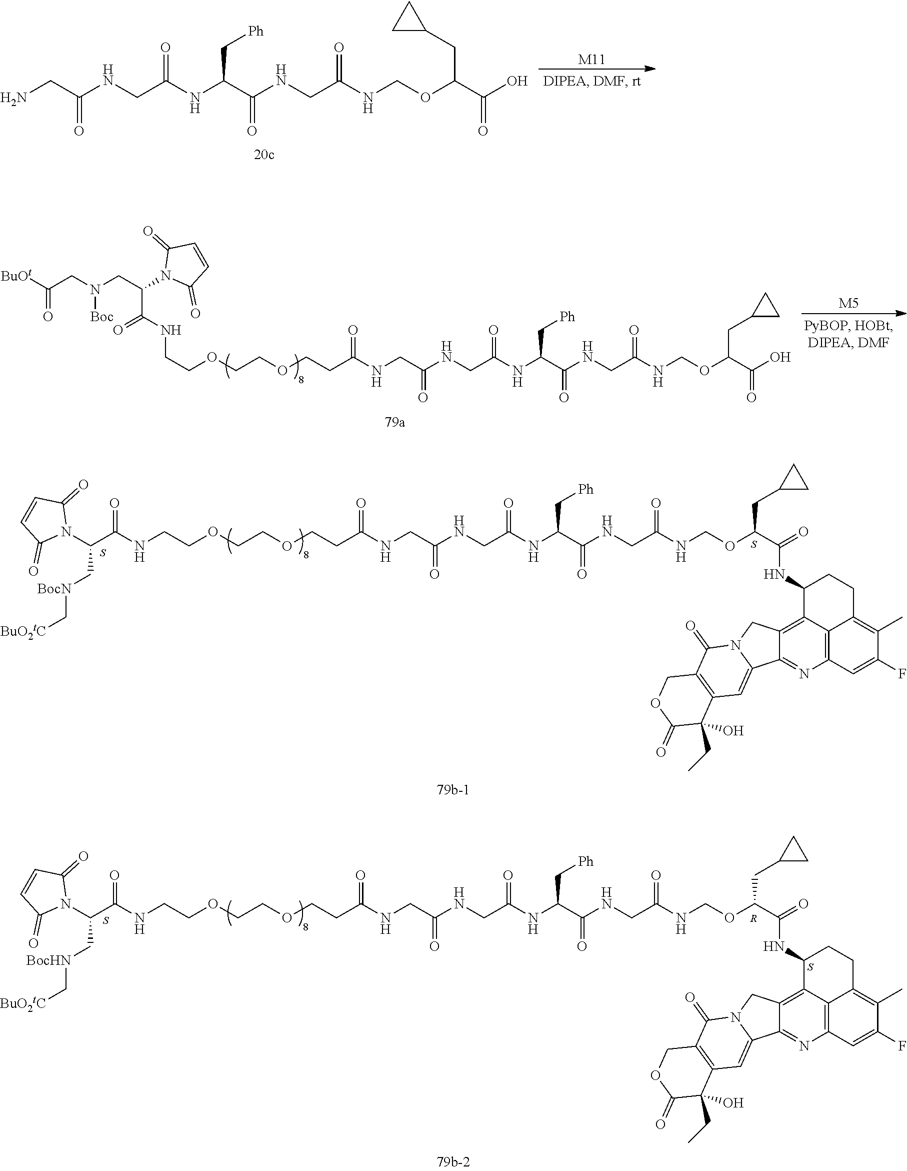

### Step 1: Synthesis of Compound 79a

Add 10 mL of DMF to M11 (1 g, 0.79 mol), cool to 0° C. in an ice-water bath, add compound 20c (377 mg, 0.79 mol) and DIPEA (154 mg, 1.19 mol), and maintain the conditions to react for 1 h. TLC monitors for completion of the reaction. The reaction solution was purified by preparative high-performance liquid chromatography (acetonitrile/purified water system), and the target peak was collected. After removing acetonitrile under reduced pressure, lyophilization was performed to obtain 783 mg of compound 79a, MS m/z: [M+H]⁺=1325.8.

### Step 2: Synthesis of Compounds 79b-1 and 79b-2

Add 79a (600 mg, 0.45 mmol), M5 (240 mg, 0.45 mmol), PyBOP (261 mg, 0.5 mmol), HOBt (68 mg, 0.5 mmol) and 10 mL of DMF to a 25 mL single-necked flask, add DIPEA (130 mg, 1 mmol) in an ice-water bath, and raise to room temperature to react for 2 h. After the completion of the reaction as detected by HIPLC, the reaction solution was purified by HIPLC to obtain the preparation liquids of compounds 79b-1 and 79b-2, and the preparation liquids were lyophilized to obtain 79b-1 (124 mg); LC-MS: [M+H]⁺=1743.0; and obtain 79b-1 (122 mg); LC-MS: [M+H]⁺=1743.0.

### Step 3: Synthesis of Compound 79

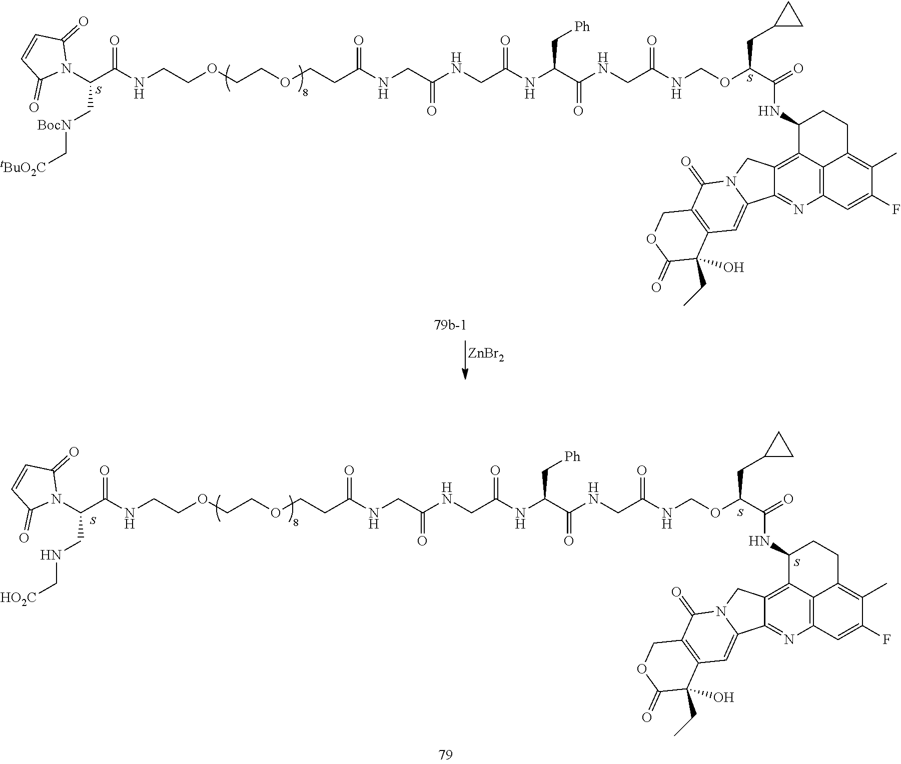

Add 79b-1 (100 mg, 0.057 mmol), zinc bromide (258 mg, 1.15 mmol) and 10 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain solid compound 79 (30 mg); LC-MS: [M+H]⁺=1586.9.

### Step 4: Synthesis of Compound 80

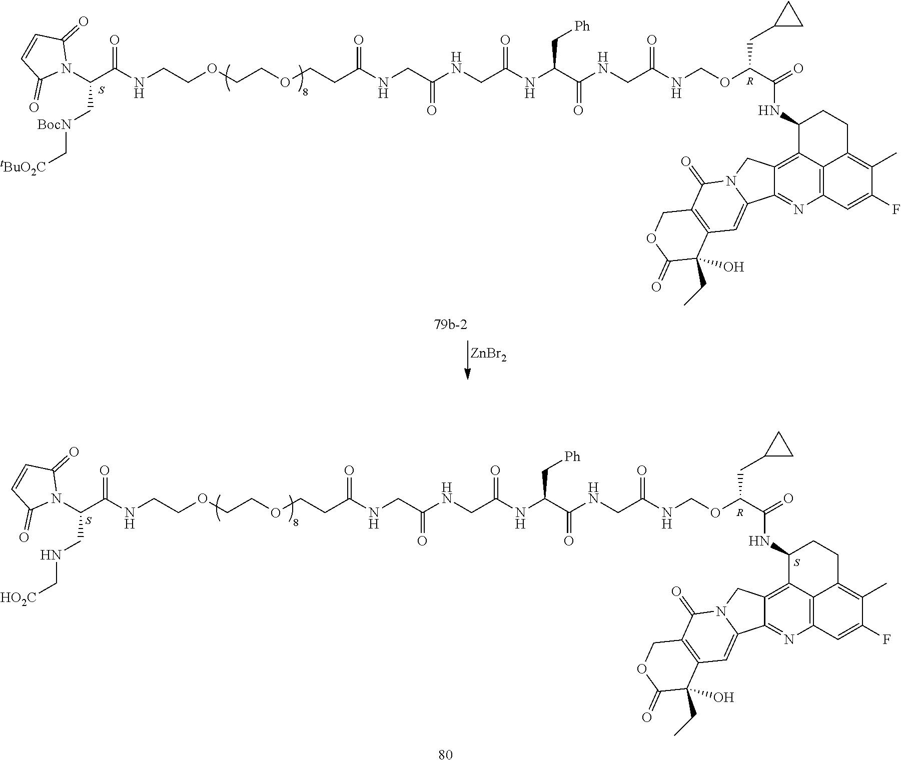

Add 79b-2 (100 mg, 0.057 mmol), zine bromide (258 mg, 1.15 mmol) and 10 mL nitromethane into a 25 mL single-necked flask and react at 40° C. for 1 h. After the completion of the reaction as detected by HPLC, the solvent was removed by concentration under reduced pressure to obtain a crude product. The crude product was purified by HPLC to obtain a product preparation liquid, which was lyophilized to obtain solid compound 80 (33 mg); LC-MS: [M+H]⁺=1587.0.

### Example 89

### Synthesis of Compound 81

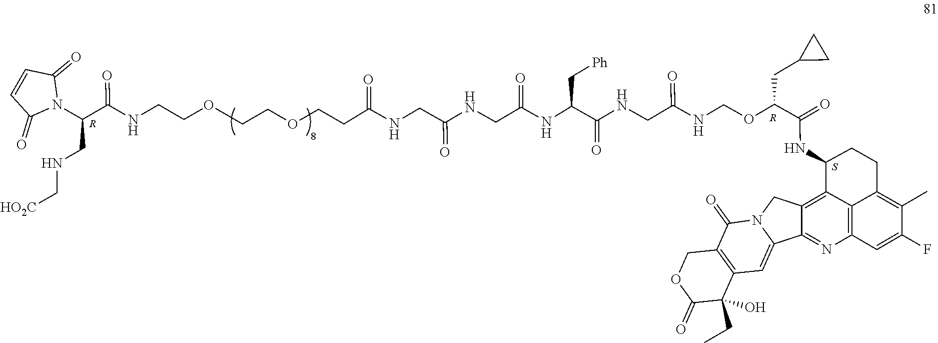

Referring to the synthetic route of Example 88, compound 81 (24 mg) was obtained; LC-MS: [M+H]⁺=1586.9.

### Example 90

### Synthesis of Compound 82

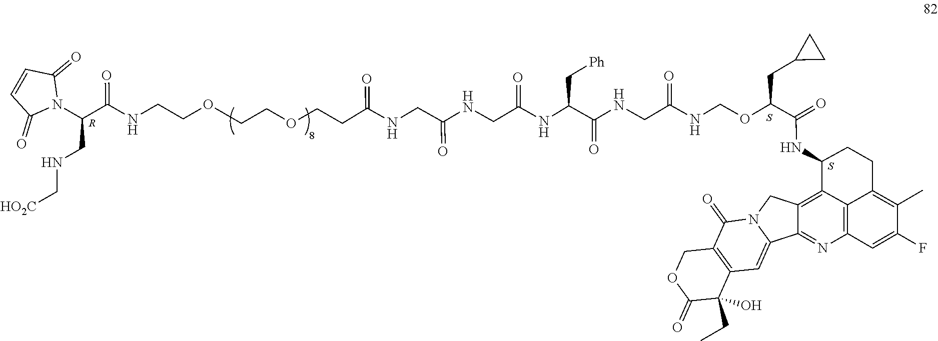

Referring to the synthetic route of Example 88, compound 82 (29 mg) was obtained; LC-MS: [M+H]⁺=1586.9.

### Example 91

### 1) Expression and Purification of SI-1×6.4 Antibody:

Expi293 (Shanghai OPM Biosciences Co., Ltd.) suspension cells were used to express SI-1×6.4 antibody. The day before transfection, cells were inoculated at a density of 0.9×10⁶ cells/mL in a 1 L shake flask containing 300 mL of OPM-293 CD05 Medium (81075-001, Shanghai OPM Biosciences Co., Ltd.), culturing was performed overnight at 37° C., 5% C02, and 120 rpm in a cell culture shaker. On the next day, the antibody expression plasmid was transfected with PEI-MAX, wherein the mass ratio of plasmid:PEI-MAX was 1:3. OPM-293 ProFeed supplement was added at 5% (v/v) on the first day after transfection, and then again at 5% (v/v) on the third day after transfection, and centrifugation was performed to collect the supernatant on the sixth day after transfection.

The collected cell expression supernatant was eluted with 0.05 M sodium acetate (pH 3.6) in a Protein A affinity chromatography column (UniMab 50, Suzhou Nanomicro Technology Co., Ltd.), and the captured antibody was adjusted to pH 7.0 with 1 M Tris-HCl (pH 8.8) at 0.7/10 (v/v), and then passed through a gel filtration chromatography column SEC (Superdex 200, GE) to remove impurities such as polymers, while the antibody buffer was replaced with 20 mM PB (pH 6.5).

### Antibody SI-1×6.4:

### Light Chain Nucleic Acid Coding Sequence

SEQ ID NO: 1
GACATCTTGCTGACTCAGTCTCCAGTCATCCTGTCTGTGAGTCCAGGAGA
AAGAGTCAGTTTCTCCTGCAGGGCCAGTCAGAGTATTGGCACAAACATAC
ACTGGTATCAGCAAAGAACAAATGGTTCTCCAAGGCTTCTCATAAAGTAT
GCTTCTGAGTCTATCTCTGGGATTCCTTCCAGGTTTAGTGGCAGTGGATC
AGGGACAGATTTTACTCTTAGCATCAACAGTGTGGAGTCTGAAGATATTG
CAGATTATTACTGTCAACAAAATAATAACTGGCCAACCACGTTCGGTGCT
GGGACCAAGCTGGAGCTGAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATOCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGTTAG

### Light Chain Amino Acid Sequence

SEQ ID NO: 2
DILLTQSPVILSVSPGERVSFSCRASQSIGTNIHWYQQRTNGSPRLLIKY
ASESISGIPSRFSGSGSGTDFILSINSVESEDIADYYCQQNNNEPTIFGA
GIKLELKRTVAAPSVFIFPPSDEQLKSGTASVVCLLNNFYPREAKVQWKV
DNALQSGNSQESVTEQDSKDSTYSLSSTLILSKADYEKHKVYACEVTHQG
LSSPVTKSENRGEC

wherein the variable region is:

SEQ ID NO: 28
DILLTQSPVILSESPGERESFSCRASQSIGTNIHWYQQRTNGSPRLLIKY
ASESISGIPSRESGSGSGIDFTLSINSVESEDIADYYCQQNNNWPTTFGA
GTKLELK

where the underlined portions are, from left to right, CDR1 (SEQ ID NO: 25), CDR2 (SEQ ID NO: 26), and CDR3 (SEQ ID NO: 27), respectively.
   Nucleic Acid Coding Sequence for the Construct of the Heavy Chain and Single-Chain Fv (scFv) Structural Domain

SEQ ID NO: 3
CAGGTGCAGCTGAAGCAGTCAGGACCTGGCCTAGTGCAGCCCTCACAGA
GCCTGTCCATCACCTGCACAGTCTCTGGTTTCTCATTAACTAACTATGG
TGTACACTGGGTTCGCCAGTCTCCAGGAAAGGGTCTGGAGTGGCTGGGA
GTGATATGGAGTGGTGGAAACACAGACTATAATACACCTTTCACATCCA
GACTGAGCATCAACAAGGACAATTCCAAGAGCCAAGTTTTCTTTAAAAT
GAACAGTCTGCAATCTAATGACACAGCCATATATTACTGTGCCAGAGCC
CTCACCTACTATGATTACGAGTTTGCTTACTGGGGCCAAGGGACTCTGG
TCACTGTCTCTAGCGCTAGCACCAAGGGCCCATCGGTCTTCCCCCTGGC
ACCCTCCTCCAAGAGCACCTCTGGGGGCACAGCGGCCCTGGGCTGCCTG
GTCAAGGACTACTTCCCCGAACCGGTGACGGTGTCGTGGAACTCAGGCG
CCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGC
ACCCAGACCTACATCTGCAACGTGAATCACAAGCCCAGCAACACCAAGG
TGGACAAGAGAGTTGAGCCCAAATCTTGTGACAAAACTCACACATGCCC
ACCGTGCCCAGCACCTGAACTCCTGGGGGGACCGTCAGTCTTCCTCTTC
CCCCCAAAACCCAAGGACACCCTCATGATCTCCCGGACCCCTGAGGTCA
CATGCGTGGTGGTGGACGTGAGCCACGAAGACCCTGAGGTCAAGTTCAA
CTGGTACGTGGACGGCGTGGAGGTGCATAATGCCAAGACAAAGCCGCGG
GAGGAGCAGTACAACAGCACGTACCGTGTGGTCAGCGTCCTCACCGTCC
TGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGG
CAGCCCCGAGAACCACAGGTGTACACCCTGCCCCCATCCCGGGATGAGC
TGACCAAGAACCAGGTCAGCCTGACCTGCCTGGTCAAAGGCTTCTATCC
CAGCGACATCGCCGTGGAGTGGGAGAGCAATGGGCAGCCGGAGAACAAC
TACAAGACCACGCCTCCCGTGCTGGACTCCGACGGCTCCTTCTTCCTCT
ATAGCAAGCTCACCGTGGACAAGAGCAGGTGGCAGCAGGGGAACGTCTT
CTCATGCTCCGTGATGCATGAGGCTCTGCACAACCACTACACGCAGAAG
AGCCTCTCCCTGTCTCCGGGTGGCGGTGGAGGGTCCGGCGGTGGTGGAT
CACAGGTGCAATTGCAGGAGTCGGGGGGAGGCCTGGTCAAGCCTGGAGG
GTCCCTGAGACTCTCCTGTGCAGCCTCTGGATTCACCTTTAGTAGTTAT
TGGATGAGCTGGGTCCGCCAGGCTCCAGGGAAGGGGCTGGAGTGGGTGG
CCAACATAAACCGCGATGGAAGTTGCGAGTTACTATGTGGACTCTGTGA
AGGGCCGATTCACCATCTCCAGAGACGACGCCAAGAACTCACTGTATCT
GCAAATGAACAGCCTGAGAGCTGAGGACACGGCTGTGTATTACTGTGCG
AGAGATCGTGGGGTGGGCTACTTCGATCTCTGGGGCCGTGGCACCCTGG
TCACCGTCTCGAGCGGTGGAGGCGGTTCAGGCGGAGGTGGTTCCGGCGG
TGGCGGCTCCCAGTCTGCCCTGACTCAGCCTGCCTCCGTGTCTGGGTCT
CCTGGACAGTCGATCACCATCTCCTGCACTGGAACCAGCAGTGACGTTG
GTGGTTATAACTTTGTCTCCTGGTACCAACAACACCCAGGCAAAGCCCC
CAAACTCATGATCTATGATGTCAGTGATCGGCCCTCAGGGGTGTCTGAT
CGCTTCTCCGGCTCCAAGTCTGGCAACACGGCCTCCCTGATCATCTCTG
GCCTCCAGGCTGACGACGAGGCTGATTATTACTGCAGCTCATATGGGAG
CAGCAGCACTCATGTGATTTTCGGCGGAGGGACCAAGGTGACCGTCCTA
TAA

Amino Acid Sequence for the Construct of Heavy Chain and Single-Chain Fv (scFv) Structural Domain

SEQ ID NO: 4
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGGG
GGSGGGGSQVQLQESGGGLVKPGGSLRLSCAASGFTFSSYWMSWVRQAPG
KGLEWVANINRDGSASYYVDSVKGRFTISRDDAKNSLYLQMNSLRAEDTA
VYYCARDRGVGYGDLWGRGTLVTVSSGGGGSGGGGSGGGGSQSALTQPAS
VSGSPGQSITISCTGTSSDVGGYNFVSWYQQHPGKAPKLMIYDVSDRPSG
VSDRFSGSKSGNTASLIISGLQADDEADYYCSSYGSSSTHVIFGGGTKVT
VL

where the variable region of the heavy chain is:

SEQ ID NO: 38
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSS

where the underlined portions are, from left to right, CDR1 (SEQ ID NO: 29), CDR2 (SEQ ID NO: 30), and CDR3 (SEQ ID NO: 31), respectively.

The variable region of the heavy chain in the structural domain of the single chain Fv (scFv) is:

SEQ ID NO: 39
QVQLQESGGGLVKPGGSLRLSCAASGFTFSSYWMSWVRQAPGKGLEWVAN
INRDGSASYYVDSVKGRFTISRDDAKNSLYLQMNSLRAEDTAVYYCARDR
GVGYFDLWGRGTLVTVSS

where the underlined portions are, from left to right, CDR1 (SEQ ID NO: 32), CDR2 (SEQ ID NO: 33), and CDR3 (SEQ ID NO: 34), respectively.

The variable region of the light chain in the single-chain Fv (scFv) structural domain is:

SEQ ID NO: 40
QSALTQPASVSGSPGQSITISCTGTSSDVGGYNFVSWYQQHPGKAPKLMI
YDVSDRPSGVSDRFSGSKSGNTASLIISGLQADDEADYYCSSYGSSSTHV
IFGGGTKVTVL

where the underlined portions are, from left to right, CDR1 (SEQ ID NO: 35), CDR2 (SEQ ID NO: 36), and CDR3 (SEQ ID NO: 37), respectively.
2) Expression and purification of SI-1×22 antibody: a similar method was followed for the expression and purification of SI-1×22 antibody.

### Antibody SI-1×22:

Nucleic Acid Coding Sequence for the Construct of Light Chain and Single-Chain Fv (scFv) Structural Domain

SEQ ID NO: 9
GACATCTTGCTGACTCAGTCTCCAGTCATCCTGTCTGTGAGTCCAGGAG
AAAGAGTCAGTTTCTCCTGCAGGGCCAGTCAGAGTATTGGCACAAACAT
ACACTGGTATCAGCAAAGAACAAATGGTTCTCCAAGGCTTCTCATAAAG
TATGCTTCTGAGTCTATCTCTGGGATTCCTTCCAGGTTTAGTGGCAGTG
GATCAGGGACAGATTTTACTCTTAGCATCAACAGTGTGGAGTCTGAAGA
TATTGCAGATTATTACTGTCAACAAAATAATAACTGGCCAACCACGTTC
GGTTGTGGGACCAAGCTGGAGCTGAAACGTACGGTGGCTGCACCATCTG
TCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
TGGAAGGTGGATAACGCCCTCCAATCGGGTAACTCCCAGGAGAGTGTCA
CAGAGCAGGACAGCAAGGACAGCACCTACAGCCTCAGCAGCACCCTGAC
GCTGAGCAAAGCAGACTACGAGAAACACAAAGTCTACGCCTGCGAAGTC
ACCCATCAGGGCCTGAGCTCGCCCGTCACAAAGAGCTTCAACAGGGGAG
AGTGTGGTGGCGGCGGAAGTGGCGGTGGAGGATCCGGCGGTGGTGGATC
ACAGGTGCAGCTGCAGGAGTCGGGGGGAGGCCTGGTCAAGCCTGGAGGG
TCCCTGAGTCTCTCCTGTGCAGCCTCTGGATTCACCTTTAGTAGTTATT
GGATGAGCTGGGTCCGCCAGGCTCCAGGGAAGGGGCTGGAGTGGGTGGC
CAACATAAACCGCCATGGAAGTGCGAGTTACTATGTGGACTCTGTGAAG
GGCCGATTCACCATCTCCAGAGACGACGCCAAGAACTCACTGTATCTGC
AAATGAACAGCCTGAGAGCTGAGGACACGGCTGTGTATTACTGTGCGAG
AGATCGTGGGGTGGGCTACTTCGATCTCTGGGGCCGTGGCACCCTGGTC
ACCGTCTGAGCGGTGGAGGCGGTTCAGGCGGAGGTGGTTCCGGCGGTGG
CGGCTCCCAGTCTGCCCTGACTCAGCCTGCCTCCGTGTCTGGGTCTCCT
GGACAGTCGATCACCATCTCCTGCACTGGAACCAGCAGTGACGTTGGTG
GTTATAACTTTGTCTCCTGGTACCAACAACACCCAGGCAAAGCCCCCAA
ACTCATGATCTATGATGTCAGTGATCGGCCCTCAGGGGTGTCTGATCGC
TTCTCCGGCTCCAAGTCTGGCAACACGGCCTCCCTGATCATCTCTGGCC
TCCAGGCTGACGACGAGGCTGATTATTACTGCAGCTCATATGGGAGCAG
CAGCACTCATGTGATTTTCGGCGGAGGGACCAAGGTGACCGTCCTATAA

Amino Acid Sequence of Construct of Light Chain and Single-Chain Fv (scFv) Structural Domain

SEQ ID NO: 10
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGC
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGGGGSGGGGSGGGGSQVQLQESGGGLVKPGGSLSLS
CAASGFTFSSYWMSWVRQAPGKGLEWVANINRDGSASYYVDSVKGRFTIS
RDDAKNSLYLQMNSLRAEDTAVYYCARDRGVGYFDLWGRGTLVTVSSGGG
GSGGGGSGGGGSQSALTQPASVSGSPGQSITISCTGTSSDVGGYNFVSWY
QQHPGKAPKLMIYDVSDRPSGVSDRFSGSKSGNTASLIISGLQADDEADY
YCSSYGSSSTHVIFGGGTKVTVL

where the variable region of the light chain is:

SEQ ID NO: 49
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGC
GTKLELK

where the underlined portions are, from left to right, CDR1 (SEQ ID NO: 25), CDR2 (SEQ ID NO: 26), and CDR3 (SEQ ID NO: 27), respectively.

The variable region of the heavy chain in the structural domain of the single-chain Fv (scFv) is:

SEQ ID NO: 50
QVQLQESGGGLVKPGGSLSLSCAASGFTFSSYWMSWVRQAPGKGLEWVAN
INRDGSASYYVDSVKGRFTISRDDAKNSLYLQMNSLRAEDTAVYYCARDR
GVGYFDLWGRGTLVTVSS

where the underlined portions are, from left to right, CDR1 (SEQ ID NO: 32), CDR2 (SEQ ID NO: 33), and CDR3 (SEQ ID NO: 34), respectively.

The variable region of the light chain in the single-chain Fv (scFv) structural domain is:

SEQ ID NO: 51
QSALTQPASVSGSPGQSITISCTGTSSDVGGYNFVSWYQQHPGKAPKLMI
YDVSDRPSGVSDRFSGSKSGNTASLIISGLQADDEADYYCSSYGSSSTHV
IFGGGTKVTVL

where the underlined portions are, from left to right, CDR1 (SEQ ID NO: 35), CDR2 (SEQ ID NO: 36), and CDR3 (SEQ ID NO: 37), respectively.

### Nucleic Acid Coding Sequence for Heavy Chains

SEQ ID NO: 11
CAGGTGCAGCTGAAGCAGTCAGGACCTGGCCTAGTGCAGCCCTCACAGA
GCCTGTCCATCACCTGCACAGTCTCTGGTTTCTCATTAACTAACTATGG
TGTACACTGGGTTCGCCAGTCTCCAGGAAAGTGCCTGGAGTGGCTGGGA
GTGATATGGAGTGGTGGAAACACAGACTATAATACACCTTTCACATCCA
GACTGAGCATCAACAAGGACAATTCCAAGAGCCAAGTTTTCTTTAAAAT
GAACAGTCTGCAATCTAATGACACAGCCATATATTACTGTGCCAGAGCC
CTCACCTACTATGATTACGAGTTTGCTTACTGGGGCCAAGGGACTCTGG
TCACTGTCTCTGCTGCTAGCACCAAGGGCCCATCGGTCTTCCCCCTGGC
ACCCTCCTCCAAGAGCACCTCTGGGGGCACAGCGGCCCTGGGCTGCCTG
GTCAAGGACTACTTCCCCGAACCGGTGACGGTGTCGTGGAACTCAGGCG
CCCTGACCACGGCGTGCACACCTTCCCGGCTGTCCTACAGTCCTCAGGA
CTCTACTCCCTCAGCAGCGTGGTGACCGTGCCCTCCAGCAGCTTGGGCA
CCCAGACCTACATCTGCAACGTGAATCACAAGCCCAGCAACACCAGGTG
GACAAGAGAGTTGAGCCCAAATCTTGTGACAAAACTCACACATGCCCAC
CGTGCCCAGCACCTGAACTCCTGGGGGGACCGTCAGTCTTCCTCTTCCC
CCCAAAACCCAAGGACACCCTCATGATCTCCCGGACCCCTGAGGTCACA
TGCGTGGTGGTGGACGTGAGCCACGAAGACCCTGAGGTCAAGTTCAACT
GGTACGTGGACGGCGTGGAGGTGCATAATGCCAAGACAAAGCCGCGGGA
GGAGCAGTACAACAGCACGTACCGTGTGGTCAGCGTCCTCACCGTCCTG
CACCAGGACTGGCTGAATGGCAAGGAGTACAAGTGCAAGGTCTCCAACA
AAGCCCTCCCAGCCCCCATCGAGAAAACCATCTCCAAAGCCAAAGGGCA
GCCCCGAGAACCACAGGTGTACACCCTGCCCCCATCCCGGGATGAGCTG
ACCAAGAACCAGGTCAGCCTGACCTGCCTGGTCAAAGGCTTCTATCCCA
GCGACATCGCCGTGGAGTGGGAGAGCAATGGGCAGCCGGAGAACAACTA
CAAGACCACGCCTCCCGTGCTGGACTCCGACGGCTCCTTCTTCCTCTAT
AGCAAGCTCACCGTGGACAAGAGCAGGTGGCAGCAGGGGAACGTCTTCT
CATGCTCCGTGATGCATGAGGCTCTGCACAACCACTACACGCAGAAGAG
CCTCTCCCTGTCTCCGGGTTAA

### Amino Acid Sequence of the Heavy Chain

SEQ ID NO: 12
QVQLKQSGPGLVQPSQSLSITCTVSGFSLTNYGVHWVRQSPGKCLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG

where the variable region of the heavy chain is:

SEQ ID NO: 52
QVQLKQSGPGLVQPSQSLSITCTVSGFSLTNYGVHWVRQSPGKCLEWLGV
IWSGGNTDYNTPFTSRLSINKDNSKSQVFFKMNSLQSNDTAIYYCARALT
YYDYEFAYWGQGTLVTVSA

where the underlined portions are, from left to right, CDR1 (SEQ ID NO: 29), CDR2 (SEQ ID NO: 30), and CDR3 (SEQ ID NO: 31), respectively.
(3) Expression and purification of SI-1×24 antibody: a similar method was followed for the expression and purification of SI-1×24 antibody.

### Antibody SI-1×24:

### Light Chain Nucleic Acid Coding Sequence

SEQ ID NO: 13
GACATCTTGCTGACTCAGTCTCCAGTCATCCTGTCTGTGAGTCCAGGAG
AAAGAGTCAGTTTCTCCTGCAGGGCCAGTCAGAGTATTGGCACAAACAT
ACACTGGTATCAGCAAAGAACAAATGGTTCTCCAAGGCTTCTCATAAAG
TATGCTTCTGAGTCTATCTCTGGGATTCCTTCCAGGTTTAGTGGCAGTG
GATCAGGGACAGATTTTACTCTTAGCATCAACAGTGTGGAGTCTGAAGA
TATTGCAGATTATTACTGTCAACAAAATAATAACTGGCCAACCACGTTC
GGTGCTGGGACCAAGCTGGAGCTGAAACGTACGGTGGCTGCACCATCTG
TCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
TGGAAGGTGGATAACGCCCTCCAATCGGGTAACTCCCAGGAGAGTGTCA
CAGAGCAGGACAGCAAGGACAGCACCTACAGCCTCAGCAGCACCCTGAC
GCTGAGCAAAGCAGACTACGAGAAACACAAAGTCTACGCCTGCGAAGTC
ACCCATCAGGGCCTGAGCTCGCCCGTCACAAAGAGCTTCAACAGGGGAG
AGTGTTAG

### Light Chain Amino Acid Sequence

SEQ ID NO: 14
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTTFG
AGTKLELKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

wherein the variable region is:

SEQ ID NO: 28
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELK

where the underlined portions are, from left to right, CDR1 (SEQ ID NO: 25), CDR2 (SEQ ID NO: 26), and CDR3 (SEQ ID NO: 27), respectively.
   Nucleic Acid Coding Sequence for the Construct of Heavy Chain and Single-Chain Fv (scFv) Structural Domain

SEQ ID NO: 15
CAGGTGCAGCTGCAGGAGTCGGGGGGAGGCCTGGTCAAGCCTGGAGGGTCCCTGAG
ACTCTCCTGTGCAGCCTCTGGATTCACCTTTAGTAGTTATTGGATGAGCTGGGTCCGC
CAGGCTCCAGGGAAGGGGCTGGAGTGGGTGGCCAACATAAACCGCGATGGAAGTGC
GAGTTACTATGTGGACTCTGTGAAGGGCCGATTCACCATCTCCAGAGACGACGCCAA
GAACTCACTGTATCTGCAAATGAACAGCCTGAGAGCTGAGGACACGGCTGTGTATTA
CTGTGCGAGAGATCGTGGGGTGGGCTACTTCGATCTCTGGGGCCGTGGCACCCTGGT
CACCGTCTCGAGCGGTGGAGGCGGTTCAGGCGGAGGTGGTTCCGGCGGTGGCGGCT
CCCAGTCTGCCCTGACTCAGCCTGCCTCCGTGTCTGGGTCTCCTGGACAGTCGATCAC
CATCTCCTGCACTGGAACCAGCAGTGACGTTGGTGGTTATAACTTTGTCTCCTGGTAC
CAACAACACCCAGGCAAAGCCCCCAAACTCATGATCTATGATGTCAGTGATGGGCCC
TCAGGGGTGTCTGATCGCTTCTCCGGCTCCAAGTCTGGCAACACGGCCTCCCTGATC
ATCTCTGGCCTCCAGGCTGACGACGAGGCTGATTATTACTGCAGCTCATATGGGAGCA
GCAGCACTCATGTGATTTTCGGCGGAGGGACCAAGGTGACCGTCCTAGGCGGTGGAG
GATCCGGCGGTGGTGGATCACAGGTGCAGCTGAAGCAGTCAGGACCTGGCCTAGTG
CAGCCCTCACAGAGCCTGTCCATCACCTGCACAGTCTCTGGTTTCTCATTAACTAACT
ATGGTGTACACTGGGTTCGCCAGTCTCCAGGAAAGGGTCTGGAGTGGCTGGGAGTGA
TATGGAGTGGTGGAAACACAGACTATAATACACCTTTCACATCCAGACTGAGCATCAA
CAAGGACAATTCCAAGAGCCAAGTTTTCTTTAAAATGAACAGTCTGCAATCTAATGAC
ACAGCCATATATTACTGTGCCAGAGCCCTCACCTACTATGATTACGAGTTTGCTTACTG
GGGCCAAGGGACTCTGGTCACTGTCTCTAGCGCTAGCACCAAGGGCCCATCGGTCTT
CCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCA
GCAGCGTGGTGACCGTGCCCTCCAGCAGCTTGGGCACCCAGACCTACATCTGCAACG
TGAATCACAAGCCCAGCAACACCAAGGTGGACAAGAGAGTTGAGCCCAAATCTTGT
GACAAAACTCACACATGCCCACCGTGCCCAGCACCTGAACTCCTGGGGGGACCGTC
AGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACT
GGTACGTGGACGGCGTGGAGGTGCATAATGCCAAGACAAAGCGGCGGGAGGAGCAG
TACAACAGCACGTACCGTGTGGTCAGCGTCCTCACCGTCCTGCACCAGGACTGGCTG
AATGGCAAGGAGTACAAGTGCAAGGTCTCCAACAAAGCCCTGCCAGCCCCCATCGA
GAAAACCATCTCCAAAGCCAAAGGGCAGCCCCGAGAACCACAGGTGTACACCCTGC
CCCCATCCCGGGATGAGCTGACCAAGAACCAGGTCAGCCTGACCTGCCTGGTCAAAG
GCTTCTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
TAA

Amino Acid Sequence of the Construct of the Heavy Chain and Single-Chain Fv (scFv) Structural Domain

SEQ ID NO: 16
QVQLQESGGGLVKPGGSLRLSCAASGFTFSSYQMSWVRQAPGKGLEWVANINRDGSASY
YVDSVKGRFTISRDDAKNSLYLQMNSLRAEDTAVYYCARDRGVGYFDLWGRGTLVTVSSG
GGGSGGGGSGGGGSQSALTQPASVSGSPGQSITISCTGTSSDVGGYNFVSWYQQHPGKA
PKLMIYDVSDRPSGVSDRFSGSKSGNTASLLIISGLQADDEADYYYCSSYGSSSTHVIFGGGTK
VTVLGGGGSGGGGSQVQLKQSGPGLVQPSQSLSITCTVSGFSLTNYGVHWVRQSPGKGL
EWLGVIWSGGNTDYNTPFTSRLSINKDNSKSQVFFKMNSLQSNDTAIYYCARALTYYDYEF
AYWGQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKSC
DKTHTCPPCPAPELLGGPSVFLFPPFPKDTLMISRTPEVTCVVVDVSHEDPEVKFNW
YVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSPG

where the variable region of the heavy chain is:

SEQ ID NO: 38
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCAR
ALTYYDYEFAYWGQGTLVTVSS

where the underlined portions are, from left to right, CDR1 (SEQ ID NO: 29), CDR2 (SEQ ID NO: 30), and CDR3 (SEQ ID NO: 31), respectively.

The variable region of the heavy chain in the structural domain of the single-chain Fv (scFv) is:

SEQ ID NO: 39
QVQLQESGGGLVKPGGSLRLSCAASGFTSSYWMSWVRQAPGKGLEWVA
NINRDGSASYYVDSVKGRFTISRDDAKNSLYLQMNSLRAEDTAVYYCA
RDRGVGYFDLWGRGTLVTVSS

where the underlined portions are, from left to right, CDR1 (SEQ ID NO: 32), CDR2 (SEQ ID NO: 33), and CDR3 (SEQ ID NO: 34), respectively.

The variable region of the light chain in the single-chain Fv (scFv) structural domain is:

SEQ ID NO: 40
QSALTQPASVSGSPGQSITISCTGTSSDVGGYNFVSWYQQHPGKAPKLM
IYDVSDRPSGVSDRFSGSKSGNTASLIISGLQADDEADYYCSSYGSSST
HVIFGGGTKVTVL

where the underlined portions are, from left to right, CDR1 (SEQ ID NO: 35), CDR2 (SEQ ID NO: 36), and CDR3 (SEQ ID NO: 37), respectively.

### Example 92

### 1) Expression and Purification of SI-1×4 Antibody:

(Shanghai OPM Biosciences Co., Ltd.) suspension cells were used to express SI-1×4 antibody. The day before transfection, cells were inoculated at a density of 0.9×10⁶ cells/mL in a 1 L shake flask containing 300 mL of OPM-293 CD05 Medium (81075-001, Shanghai OPM Biosciences Co., Ltd.), and cultured overnight at 37° C., 5% CO₂, and 120 rpm in a cell culture shaker. On the next day, the antibody expression plasmid was transfected with PEI-MAX, wherein the mass ratio of plasmid:PEI-MAX was 1:3. OPM-293 ProFeed supplement was added at 5% (v/v) on the first day after transfection, and then again at 5% (v/v) on the third day after transfection, and then centrifugation was performed to collect the supernatant on the sixth day after transfection.

The collected cell expression supernatant was collected and eluted with 0.05 M sodium acetate (pH 3.6) in a Protein A affinity chromatography column (UniMab 50, Suzhou Nanomicro Technology Co., Ltd.), and the captured antibody was adjusted to pH 7.0 with 1 M Tris-HCl (pH 8.8) at 0.7/10 (v/v), and then passed through a gel filtration chromatography column SEC (Superdex 200, GE) to remove impurities such as polymers, while the antibody buffer was replaced with 20 mM PB (pH 6.5).

### Antibody SI-1×4:

### Light Chain Nucleic Acid Coding Sequence

SEQ ID NO: 5
GATATTCAAATGACTCAATCTCCTTCTTCTCTTTCTGCTTCTGTTGGTGATCGTGTTACT
ATTACTTGTCGTTCTTCTCAAAATATTGTTCATTCTAATGGTAATACTTATCTTGATTGGT
ATCAACAAACTCCTGGTAAAGCTCCTAAACTTCTTATTTATAAAGTTTCTAATCGTTTT
TCTGGTGTTCCTTCTCGGTTTTTCTGGTTCTGGTTTCTGGTACTGATTTTACTTTTACTATT
TCTTCTCTTCAACCTGAAGATATTGCTACTTATTATTGTTTTCAATATTCTCATGTTCCTT
GGACTTTTGGTCAAGGTACTAAACTTCAAATTACTCGTACGGTGGCTGCACCATCTGT
CTTCATCTTCCCGCCATCTGATGAGCAGTTGAAATCTGGAACTGCCTCTGTTGTGTGC
CTGCTGAATAACTTCTATCGCAGAGAGGCCAAAGTACAGTGGAAGGTGGATAACGCC
CTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAG
TCTACGCCTGCGAAGTCACCCATCAGGGCCTGAGCTCGCCCGTCACAAAGAGCTTCA
ACAGGGGAGAGTGTTAG

### Light Chain Amino Acid Sequence

SEQ ID NO: 6
DIQMTQSPSSLSASVGDRVTITCRSSQNIVHSNGNTYLDWYQQTPGKAPK
LLIYKVSNRFSGVPSRFSGSGSGTDFTFTISSLQPEDIATYYCFQYSHVP
WTFGQGTKLQITRTVAAPSVIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

wherein the variable region is:

SEQ ID NO: 44
DIQMTQSPSSLSASVGDRVTITCRSSQNIVHSNGNTYLDWYQQTPGKAPK
LLIYKVSNRFSGVPSRFSGSGSGTDFTFTISSLQPEDIATYYCFQYSHVP
WTFGQGTKLQIT

where the underlined portions are, from left to right, CDR1 (SEQ ID NO: 41), CDR2 (SEQ ID NO: 42), and CDR3 (SEQ ID NO: 43), respectively.
   Nucleic Acid Coding Sequence for the Construct of the Heavy Chain and Single-Chain Fv (scFv) Structural Domain

SEQ ID NO: 7
CAGGTGCAGCTGCAGCAGAGCGGCGCCGAGGTGAAGAAGCCCGGCAGCAGCGTGA
AGGTGAGCTGCAAGGCCAGCGGCTACACCTTCACCAACTACTACATCTACTGGGTGC
GGCAGGCCCCCGGCCAGGGCCTGGAGTGGATCGGCGGCATCAACCCCACCAGCGGC
GGCAGCAACTTCAACGAGAAGTTCAAGACCCGGGTGACCATCACCGCCGACGAGAG
CAGCACCACCGCCTACATGGAGCTGAGCAGCCTGCGGAGCGAGGACACCGCCTTCTA
CTTCTGCACCCGGCAGGGCCTGTGGTTCGACAGCGACGGCCGGGGCTTCGACTTCTG
GGGCCAGGGCACCACCGTGACCGTGAGCAGCGCTAGCACCAAGGGCCCATCGGTCT
TCCCCCTGGCACCCTCCTCCAAGAGCACCTCTGGGGGCACAGCGGCCCTGGGCTGCC
TGGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTGCCTCA
GCAGCGTGGTGACCGTGCCCTCCAGCAGCTTGGGCACCCAGACCTACATCTGCAACG
TGAATCACAAGCCCAGCAACACCAAGGTGGACAAGAGAGTTGAGCCCAAATCTTGT
GACAAAACTCACACATGCCCACCGTGCCCAGCACCTGAACTCCTGGGGGGACCGTC
AGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACACCCTGC
CCCCATCCCGGGATGAGCTGACCAAGAACCAGGTCAGCCTGACCTGCCTGGTCAAAG
GCTTCTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
GGCGGTGGAGGGTCCGGCGGTGGTGGATCACAGGTGCAATTGCAGGAGTCGGGGGG
AGGCCTGGTCAAGCCTGGAGGGTCCCTGAGACTCTCCTGTGCAGCCTCTGGATTCAC
CTTTAGTAGTTATTGGATGAGCTGGGTCCGCCAGGCTCCAGGGAAGGGGCTGGAGTG
GGTGGCCAACATAAACCGCGATGGAAGTGCGAGTTACTATGTGGACTCTGTGAAGGG
CCGATTCACCATCTCCAGAGACGACGCCAAGAACTCACTGTATCTGCAAATGAACAG
CCTGAGAGCTGAGGACACGGCTGTGTATTACTGTGCGAGAGATCGTGGGGTGGGCTA
CTTCGATCTCTGGGGCCGTGGCACCCTGGTCACCGTCTCGAGCGGTGGAGGCGGTTC
AGGCGGAGGTGGTTCCGGCGGTGGGGGCTCCCAGTCTGCCCTGACTCAGCCTGCCTC
CGTGTCTGGGTCTCCTGGACAGTCGATCACCATCTCCTGCACTGGAACCAGCAGTGA
CGTTGGTGGTTATAACTTTGTCTCCTGGTACCAACAACACCCAGGCAAAGCCCCCAA
ACTCATGATCTATGATGTCAGTGATCGGCCCTCAGGGGTGTCTGATCGCTTCTCCGGC
TCCAAGTCTGGCAACACGGCCTCCCTGATCATCTCTGGCCTCCAGGCTGACGACGAG
GCTGATTATTACTGCAGCTCATATGGGAGCAGCAGCACTCATGTGATTTTCGGCGGAG
GGACCAAGGTGACCGTCCTATAA

Amino Acid Sequence of the Construct of Heavy Chain and Single Chain Fv (scFv) Structural Domain

SEQ ID NO: 8
QVQLQQSGAEVKKPGSSVKVSCKASGYTFTNYYIYWVRQAPGQGLEWIGGINPTSGGSNF
NEKFKTRVTITADESSTTAYMELSSLRSEDTAFYFCTRQGLWFDSDGRGFDFWGQGTTVTV
SSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNA
KTKPREEQYNSTYRVVSVLTVLHQDWLNGKBYKCKVSNKALPAPIEKTISKAKGQPR
EPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGGGGGSGGGGSQV
QLQESGGGLVKPGGSLRLSCAASGFTFSSYWMSWVRQAPGKGLEWVANINRDGSASYYV
DSVKGRFTISRDDAKNSLYLQMNSLRAEDTAVYYCARDRGVGYFDLWGRGTLVTVSSGGG
GSGGGGSGGGSQSALTQPASVSGSPGQSITISCTGTSSDVGGYNFVSWYQQHPGKAPKL
MIYDVSDRPSGVSDRFSGSKSGNTASLIISGLQADDEADYYCSSYGSSSTHVIFGGGTKVTVL

where the variable region of the heavy chain is:

SEQ ID NO: 48
QVQLQQSGAEVKKPGSSVKVSCKASGYTFTNYYIYWVRQAPGQGLEWIG
GINPTSGGSNFNEKFKTRVTITADESSTTAYMELSSLRSEDTAFYFCTR
QGLWFDSDGRGFDFWGQGTTVTVSS

where the underlined portions are, from left to right, CDR1 (SEQ ID NO: 45), CDR2 (SEQ ID NO: 46), and CDR3 (SEQ ID NO: 47), respectively.

The variable region of the heavy chain in the structural domain of the single-chain Fv (scFv) is:

SEQ ID NO: 39
QVQLQESGGLVKPGGSLRLSCAASGFTFSSYWMSWVRQAPGKAPGKGLEW
VANINRDGSASYYVDSVKGRFTISRDDDAKNSLYLQMNSLRAEDTALVYY
CARDRGVGYFDLWGRGTLVTVSS

where the underlined portions are, from left to right, CDR1 (SEQ ID NO: 32), CDR2 (SEQ ID NO: 33), and CDR3 (SEQ ID NO: 34), respectively.

The variable region of the light chain in the single-chain Fv (scFv) structural domain is:

SEQ ID NO: 40
QSALTQPASVSGSPGQSITISCTGTSSDVGGYNFVSWYQQHPGKAPKLMI
YDVSDRPSGVSDRFSGSKSGNTASLIISGLQADDEADYYCSSYGSSSTHV
IFGGGTKVTVL

where the underlined portions are, from left to right, CDR1 (SEQ ID NO: 35), CDR2 (SEQ ID NO: 36), and CDR3 (SEQ ID NO: 37), respectively.
2) Expression and purification of SI-1×25 antibody: a similar method was followed for the expression and purification of SI-1×25 antibody.

### Antibody SI-1×25:

Nucleic Acid Coding Sequence for the Construct of Light Chain and Single-Chain Fv (scFv) Structural Domain

SEQ ID NO: 17
CAGGTGCAGCTGCAGGAGTCGGGGGGAGGCCTGGTCAAGCCTGGAGGGTCCCTGAG
TCTCTCCTGTGCAGCCTCTGGATTCACCTTTAGTAGTTATTGGATGAGCTGGGTCCGCC
AGGCTCCAGGGAAGGGGCTGGAGTGGGTGGCCAACATAAACCGCGATGGAAGTGCG
AGTTACTATGTGGACTCTGTGAAGGGCCGATTCACCATCTCCAGAGACGACGCCAAG
AACTCACTGTATCTGCAAATGAACAGCCTGAGAGCTGAGGACACGGCTGTGTATTAC
TGTGCGAGAGATCGTGGGGGGGCTACTTCGATCTCTGGGGCCGTGGCACCCTGGTC
ACCGTCTCGAGCGGTGGAGGCGGTTCAGGCGGAGGTGGTTCCGGGGGTGGCGGCTC
CCAGTCTGCCCTGACTCAGCCTGCCTCCGTGTCTGGGTCTCCTGGACAGTCGATCACC
ATCTCCTGCACTGGAACCAGCAGTGACGTTGGTGGTTATAACTTTGTCTCCTGGTACC
AACAACACCCAGGCAAAGCCCCCAAACTCATGATCTATGATGTCAGTGATCGGCCCT
CAGGGGTGTCTGATCGCTTCTCCGGCTCCAAGTCTGGCAACACGGCCTCCCTGATCAT
CTCTGGCCTCCAGGCTGACGACGAGGCTGATTATTACTGCAGCTCATATGGGAGCAGC
AGCACTCATGTGATTTTCGGCGGAGGGACCAAGGTGACCGTCCTAGGCGGTGGAGG
ATCCGGCGGTGGTGGATCAGACATCTTGCTGACTCAGTCTCCAGTCATCCTGTCTGTG
AGTCCAGGAGAAAGAGTCAGTTTCTCCTGCAGGGCCAGTCAGAGTATTGGCACAAA
CATACACTGGTATCAGCAAAGAACAAATGGTTCTCCAAGGCTTCTCATAAAGTATGCT
TCTGAGTCTATCTCTGGGATTCCTTCCAGGTTTAGTGGCAGTGGATCAGGGACAGATT
TTACTCTTAGCATCAACAGTGTGGAGTCTGAAGATATTGCAGATTATTACTGTCAACA
AAATAATAACTGGCCAACCACGTTCGGTTGTGGGACCAAGCTGGAGCTGAAACGTAC
GGTGGCTGCACCATCTGTCTTCATCTTCCCGCCATCTGATGAGCAGTTGAAATCTGGA
ACTGCCTCTGTTGTGTGCCTGCTGAATAACTTCTATGCCAGAGAGGCCAAAGTACAGT
GGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGA
CTACGAGAAACACAAAGTCTACGCCTGCGAAGTCACCCATCAGGGCCTGAGCTCGCC
CGTCACAAAGAGCTTCAACAGGGGAGAGTGTTAG

Amino Acid Sequence of Construct of Light Chain and Single-Chain Fv (scFv) Structural Domain

SEQ ID NO: 18
QVQLQESGGGLVKPGGSLSCAASGFTFSSYWMSWVRQAPGKGLEWVANINRDGSASY
YVDSVKGRFTISRDDAKNSLYLQMNSLRAEDTAVYYCARDRGVGYFDLWGRGTLVTVSSG
GGGSGGGGSGGGGQSALTQPASVSGSPGQSITISCTGTSSDVGGYNFVSWYQQHPGKA
PKLMIYDVSDRPSGVSDRFSGSKSGNTASLIISGLQADDEADYYCSSYGSSSTHVIFGGGTK
VTVLGGGGSGGGGSDILLTQSPVILSVSPGERVSFSCRASQSIGTNIHWYQQRTNGSPRLLI
KYASESISGIPSRFSGSGSGTDFTLSINSVESEDIADYYCQQNNNWPTTFGCGTKLELKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC

where the variable region of the light chain is:

SEQ ID NO: 49
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GCGTKLELK

where the underlined portions are, from left to right, CDR1 (SEQ ID NO: 25), CDR2 (SEQ ID NO: 26), and CDR3 (SEQ ID NO: 27), respectively.

The variable region of the heavy chain in the structural domain of the single-chain Fv (scFv) is:

SEQ ID NO: 50
QVQLQESGGGLVKPGGSLSLSCAASGFTFSSYWMSWVRQAPGKGLEWVA
NINRDGSASYYVDSVKGRFTISRDDAKNSLYLQMNSLRAEDTAVYYCAR
DRGVGYFDLWGRGTLVTVSS

where the underlined portions are, from left to right, CDR1 (SEQ ID NO: 32), CDR2 (SEQ ID NO: 33), and CDR3 (SEQ ID NO: 34), respectively.

The variable region of the light chain in the single-chain Fv (scFv) structural domain is:

SEQ ID NO: 51
QSALTQPASVSGSPGQSITISCTGTSSDVGGYNFVSWYQQHPGKAPKLM
IYDVSDRPSGVSDRFSGSKSGNTASLIISGLQADDEADYYCSSYGSSST
HVIFGGGTKVTVL

where the underlined portions are, from left to right, CDR1 (SEQ ID NO: 35), CDR2 (SEQ ID NO: 36), and CDR3 (SEQ ID NO: 37), respectively.

### Nucleic Acid Coding Sequence for the Heavy Chain

SEQ ID NO: 19
CAGGTGCAGCTGAAGCAGTCAGGACCTGGCCTAGTGCAGCCCTCACAGAGGCTGTC
CATCACCTGCACAGTCTCTGGTTTCTCATTAACTAACTATGGTGTACACTGGGTTCGC
CAGTCTCCAGGAAAGTGCCTGGAGTGGCTGGGAGTGATATGGAGTGGTGGAAACAC
AGACTATAATACACCTTTCACATCCAGACTGAGCATCAACAAGGACAATTCCAAGA
GCCAAGTTTTCTTTAAAATGAACAGTCTGCAATCTAATGACACAGCCATATATTACT
GTGCCAGAGCCCTCACCTACTATGATTACGAGTTTGCTTACTGGGGCCAAGGGACTC
TGGTCACTGTCTCTGCTGCTAGCACCAAGGGCCCATCGGTCTTCCCCCTGGCACCCT
CCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGAC
CGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACA
CATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTG
GTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCT
CCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCC
AGCGACATCGCCGTGGAGTGGGAGAGCAATGGGCAGCCGGAGAACAACTACAAGA
CCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGG
CTCTGCACAACCACTACACGCAGAAGAGCCTCTCCCTGTCTGCGGGTTAA

### Amino Acid Sequence of the Heavy Chain

SEQ ID NO: 20
QVQLKQSGPGLVQPSQSLSITCTVSGFSLTNYGVHWVRQSPGKCLEWLGVIWSGGNTDYN
TPFTSRLSINKDNSKSQVFFKMNSLQSNDTAIYYCARALTYYDYEFAYWGQGTLVTVSAAS
TKGPSVFPLAPSSKSTSGGTAALGCLVDKYFPEPVTVSWNSGALTSGVHTFPAVLQS
SGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPG

where the variable region of the heavy chain is:

SEQ ID NO: 52
QVQLKQSGPGLVQPSQSLSITCTVSGFLTNYGVHWVRQSPGKCLEWLGVI
WSGGNTDYNTPFTSRLSINKDNSKSQVFFKMNSLQSNDTAIYYCARALTY
YDYEFAYWGQGTLVTVSA

where the underlined portions are, from left to right, CDR1 (SEQ ID NO: 29), CDR2 (SEQ ID NO: 30), and CDR3 (SEQ ID NO: 31), respectively.
(3) Expression and purification of SI-1×26 antibody: a similar method was followed for the expression and purification of SI-1×26 antibody.

### Antibody SI-1×26:

Nucleic Acid Coding Sequence for the Construct of Light Chain and Single-Chain Fv (scFv) Structural Domain

SEQ ID NO: 21
GATATTCAAATGACTCAATCTCCTTCTTCTCTTTCTGCTTCTGTTGGTGATCGTGTTACT
ATTACTTGTCGTTCTTCTCAAAATATTGTTCATTCTAATGGTAATACTTATCTTGATTGGT
ATCAACAAACTCCTGGTAAAGCTCCTAAACTTCTTATTTATAAAGTTTCTAATCGTTTT
TCTGGTGTTCCTTCTCGTTTTTCTGGTTCTGGTTCTGGTACTGATTTTACTTTTACTATT
TCTTCTCTTCAACCTGAAGATATTGCTACTTATTATTGTTTTCAATATTCTCATGTTCCTT
GGACTTTTGGTTGCGGTACTAAACTTCAAATTACTCGTACGGTGGCTGCACCATCTGT
CTTCATCTTCCCGCCATCTGATGAGCAGTTGAAATCTGGAACTGCCTCTGTTGTGTGC
CTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAG
TCTACGCCTGCGAAGTCACCCATCAGGGCCTGAGCTCGCCCGTCACAAAGAGCTTCA
ACAGGGGAGAGTGTGGTGGCGGCGGAAGTGGCGGTGGAGGATCCGGCGGTGGTGG
ATCACAGGTGCAGCTGCAGGAGTCGGGGGGAGGCCTGGTCAAGCCTGGAGGGTCCC
TGAGTCTCTCCTGTGCAGCCTCTGGATTCACCTTTAGTAGTTATTGGATGAGCTGGGT
CCCCCAGGCTCCAGGGAAGGGGCTGGAGTGGGTGGCCAACATAAACCGCGATGGAA
GTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGT
ATTACTGTGCGAGAGATCGTGGGGTGGGCTACTTCGATCTCTGGGGCCGTGGCACCCT
GGTCACCGTCTCGAGCGGTGGAGGCGGTTCAGGCGGAGGTGGTTCCGGCGGTGGCG
GCTCCCAGTCTGCCCTGACTCAGCCTGCCTCCGTGTCTGGGTCTCCTGGACAGTCGAT
CACCATCTCCTGCACTGGAACCAGCAGTGACGTTGGTGGTTATAACTTTGTCTCCTGG
TACCAACAACACCCAGGCAAAGCCCCCAAACTCATGATCTATGATGTCAGTGATCGG
CCCTCAGGGGTGTCTGATCGCTTCTCCGGCTCCAAGTCTGGCAACACGGCCTCCCTG
ATCATCTCTGGCCTCCAGGCTGACGACGAGGCTGATTATTACTGCAGCTCATATGGGA
GCAGCAGCACTCATGTGATTTTCGGCGGAGGGACCAAGGTGACCGTCCTATAA

Amino Acid Sequence of the Construct of Light Chain and Single-Chain Fv (scFv) Structural Domain

SEQ ID NO: 22
DIQMTQSPSSLSASVGDRVTITCRSSQNIVHSNGNTYDWYQQTPGKAPKLLIYKVSNRFSG
VPSRFSGSGSGTDFTFTISSLQPEDIATYYCFQYSHVPWTFGCGTKLQITRTVAAPSVFIFP
PSDEQLKSGTASVVCLLNNFYPREAKVQWKVDNALQSGNSQESVTEQDSKDSTYSL
SSTLTLSKADYEKHKVYACEVTHQGLSSPVTKSFNRGECGGGGSGGGGSGGGGSQV
QLQESGGGLVKPGGSLSLSCAASGFRFSSYWMSWVRQAPGKGLEWVANINRDGSASYYVD
SVKGRFTISRDDAKNSLYLQMNSLRAEDTAVYYCARDRGVGYFDLWGRGTLVTVSS
GGGGSGGGGSGGGGSQSALTQPASVSGSPGQSITISCTGTSSDVGGYNFVSWYQQHPGKAPKLM
IYDVSDRPSGVSDRFSGSKSGNTASLIISGLOADDEADYYCSSYGSSSTHVIFGGGTKVTVL

where the variable region of the light chain is:

SEQ ID NO: 53
DIQMTQSPSSLSASVGDRVTITCRSSQNIVHSNGTYLDWYQQTPGKAPKL
LIYKVSNRFSGVPSRFSFSFSFTDFTFTISSLQPEDIATYYCFQYSHVPW
TFGCGTKLQIT

where the underlined portions are, from left to right, CDR1 (SEQ ID NO: 41), CDR2 (SEQ ID NO: 42), and CDR3 (SEQ ID NO: 43), respectively.

The variable region of the heavy chain in the structural domain of the single-chain Fv (scFv) is:

SEQ ID NO: 50
QVQLQESGGGLVKPGGSGSGSCAASGFTGSSYQMSWVRQAPGKGLEWVA
NINRDGSASYYVDSVKGRFTISRDDAKNSLYLQMNSLRAEDTAVYYCAR
DRGVGYFDLWGRGTLVTVSS

where the underlined portions are, from left to right, CDR1 (SEQ ID NO: 32), CDR2 (SEQ ID NO: 33), and CDR3 (SEQ ID NO: 34), respectively.

The variable region of the light chain in the single-chain Fv (scFv) structural domain is:

SEQ ID NO: 51
QSALTQPASVSGSPGQSITISCTGTSSDVGGYNFVSWYQQHPGKAAPKLMI
YDVSDRPSGVSDRFSGSKSGNTASLIISGLQDDEADYYCSSYGSSSTHVIF
GGGTKVTVL

where the underlined portions are, from left to right, CDR1 (SEQ ID NO: 35), CDR2 (SEQ ID NO: 36), and CDR3 (SEQ ID NO: 37), respectively.

### Nucleic Acid Coding Sequence for the Heavy Chain

SEQ ID NO: 23
CAGGTGCAGCTGCAGCAGAGCGGCGCCGAGGTGAAGAAGCCCGGCAGCAGCGTGA
AGGTGAGCTGCAAGGCCAGCGGCTACACCTTCACCAACTACTACATCTACTGGGTGC
GGCAGGCCCCCGGCCAGTGTCTGGAGTGGATCGGCGGCATCAACCCCACCAGCGGC
GGCAGCAACTTCAACGAGAAGTTCAAGACCCGGGTGACCATCACCGCCGACGAGAG
CAGCACCACCGCCTACATGGAGCTGAGCAGCCTGCGGAGCGAGGACACCGCCTTCTA
CTTCTGCACCCGGCAGGGCCTGTGGTTCGACAGCGACGGCCGGGGCTTCGACTTCTG
GGGCCAGGGCACCACCGTGACCGTGAGCAGCGCTAGCACCAAGGGCCCATCGGTCT
TCCCCCTGGCACCCTCCTCCAAGAGCACCTCTGGGGGCACAGCGGCCCTGGGCTGCC
TGGTCAAGGACTACTTCCCCGAACCGGTGACGGTGTCGTGGAACTCAGGGGCCCTGA
CCAGCGGCGTGCACACCTTCCCGGCTGTCCTACAGTCCTCAGGACTCTACTCCCTCA
GCAGCGTGGTGACCGTGCCCTCCAGCAGCTTGGGCACCCAGACCTACATCTGCAACG
TGAATCACAAGCCCAGCAACACCAAGGTGGACAAGAGAGTTGAGCCCAAATCTTGT
GACAAAACTCACACATGCCCACCGTGCCCAGCACCTGAACTCCTGGGGGGACCGTC
AGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACACGCTGC
CCCCATCCCGGGATGAGCTGACCAAGAACCAGGTCAGCCTGACCTGCCTGGTCAAAG
GCTTCTATCCCAGCGACATCGCCGTGGAGTGGGÅGAGCAATGGGCAGCCGGAGAAC
AACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
TAA

### Amino Acid Sequence of the Heavy Chain

SEQ ID NO: 24
QVQLQQSGAEVKKPGSSVKVSCKASGYTFTNYYIYWVRQAPGQCLEWIGGI
NPTSGGSNFNEKFKTRVTITADESSTAYMELSSLRSEDTAFYFCTRQGLWF
DSDGRGFDFWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGS
FFLYSKLTVDKSRWQQGNVFSCSVMHEALHNHYTQKSLSLSPG

where the variable region of the heavy chain is:

SEQ ID NO: 54
QVQLQQSGAEVKKPGSSVKVSCKASGYTFTNYYIYWVRQAPGQCLEWIG
GINPTSGGSNFNEKFKTRVTITADESSTTAYMELSSLRSEDTAFYFCTR
QGLWFDSDGRGFDFWGQGTTVTVSS

where the underlined portions are, from left to right, CDR1 (SEQ ID NO: 45), CDR2 (SEQ ID NO: 46), and CDR3 (SEQ ID NO: 47), respectively.

### Example 93

1) A sample of SI-1×6.4 antibody-drug conjugate was prepared by coupling SI-1×6.4 antibody with payload:

After cellular expression and purification by Protein A affinity chromatography and molecular sieve chromatography, the SI-1×6.4 antibody was replaced in 20 mM PB, pH 6.5 buffer, and the SI-1×6.4 antibody was concentrated or diluted to a protein concentration of 3 mg/mL. The payload was a white powder, which was dissolved to 20 mg/mL using DMA and set aside. To open the interchain disulfide bonds of SI-1×6.4 antibody, 20-fold TECP was first added according to the molecular ratio and a reaction was performed at room temperature for 3 h. Then 20-fold payload solution was added according to the molecular ratio and a reaction was performed at room temperature for 1 h. After the reaction was completed, the payload that was not coupled to SI-1×6.4 was removed by ultrafiltration using a 30 KDa ultrafiltration centrifuge tube, and the SI-1×6.4 antibody-drug conjugate sample was thus obtained.
2) A sample of SI-1×22 antibody-drug conjugate was prepared by coupling SI-1×22 antibody with payload following a similar method.
3) A sample of SI-1×24 antibody-drug conjugate was prepared by coupling SI-1×24 antibody with payload following a similar method.

### Example 94

1) A sample of SI-1×4 antibody-drug conjugate was prepared by coupling SI-1×4 antibody with payload:

After cellular expression and purification by Protein A affinity chromatography and molecular sieve chromatography, the SI-1×4 antibody was replaced in 20 mM PB, pH 6.5 buffer, and the SI-1×4 antibody was concentrated or diluted to a protein concentration of 3 mg/mL. The payload was a white powder, which was dissolved to 20 mg/mL using DMA and set aside. To open the interchain disulfide bonds of SI-1×4 antibody, 20-fold TECP was first added according to the molecular ratio and a reaction was carried out at room temperature for 3 h. Then, 20-fold payload solution was added according to the molecular ratio and a reaction was carried out at room temperature for 1 h. At the end of the reaction, the payload that was not coupled with SI-1×4 was removed by ultrafiltration using a 30 KDa ultrafiltration centrifuge tube to obtain a sample of SI-1×4 antibody-drug conjugate.
2) A sample of SI-1×25 antibody-drug conjugate was prepared by coupling SI-1×25 antibody with payload following a similar method.
3) A sample of SI-1×26 antibody-drug conjugate was prepared by coupling SI-1×26 antibody with payload following a similar method.

### Example 95

ADC-1 was prepared according to the general-purpose coupling method of Example 93,

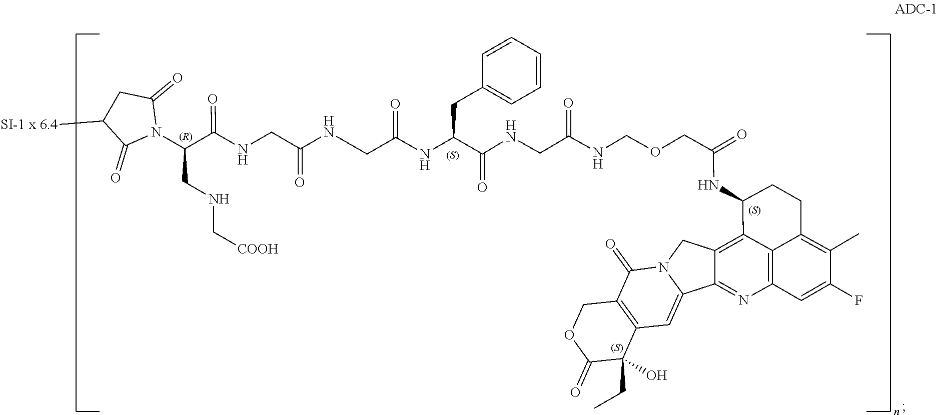

### Example 96

ADC-2 was prepared according to the general-purpose coupling method of Example 93,

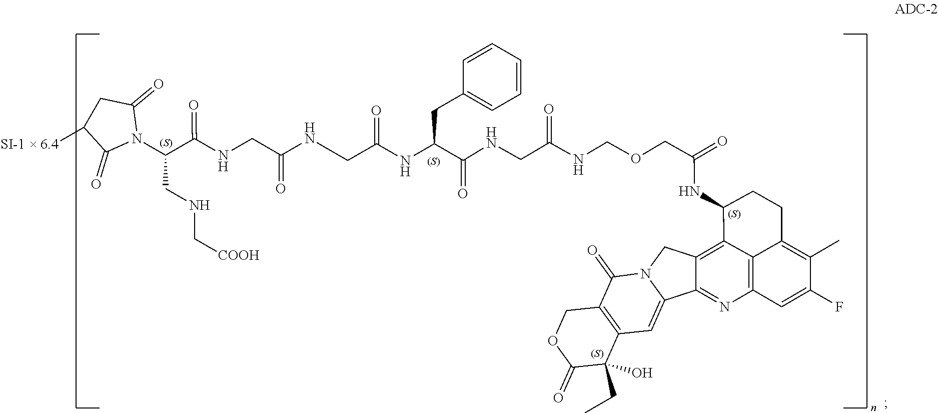

### Example 97

ADC-3 was prepared according to the general-purpose coupling method of Example 93,

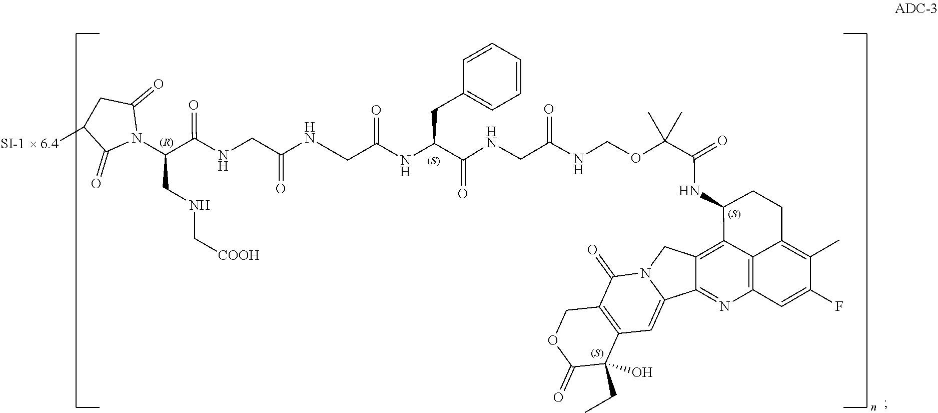

### Example 98

ADC-4 was prepared according to the general-purpose coupling method of Example 93,

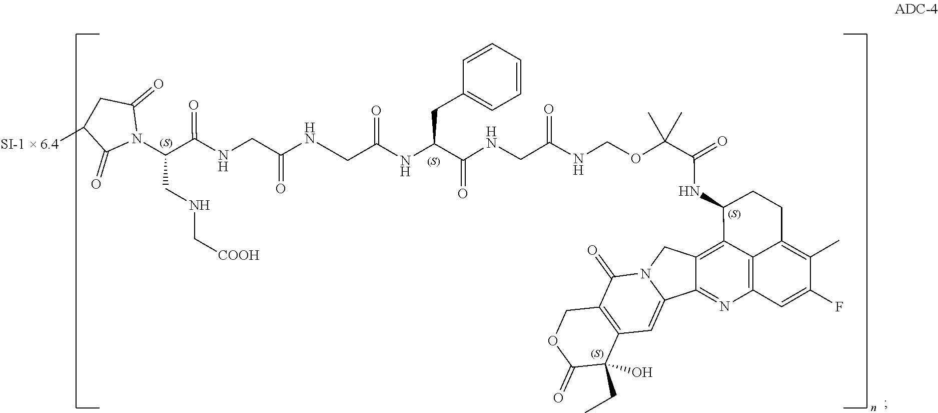

### Example 99

ADC-5 was prepared according to the general-purpose coupling method of Example 93,

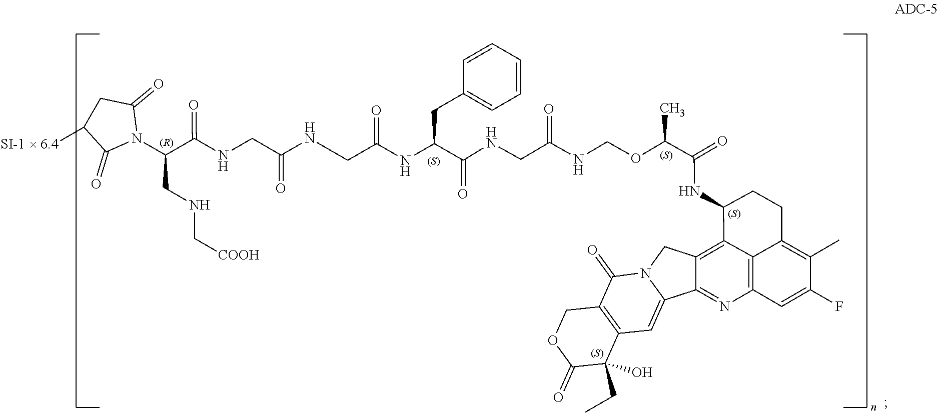

### Example 100

ADC-6 was prepared according to the general-purpose coupling method of Example 93,

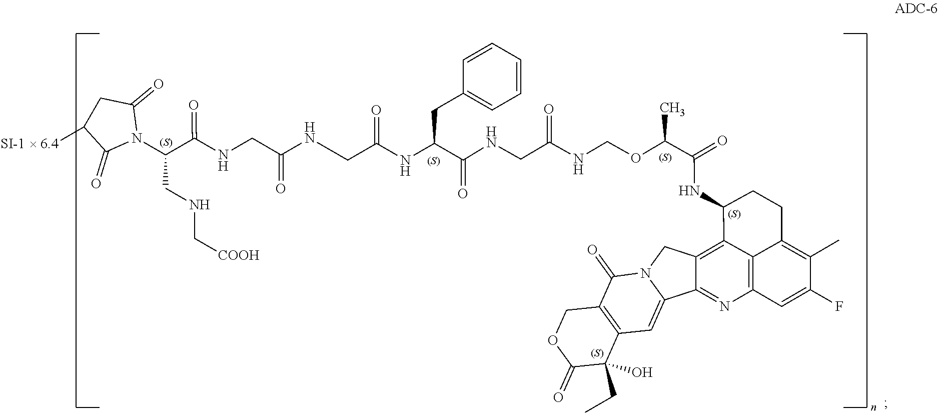

### Example 101

ADC-7 was prepared according to the general-purpose coupling method of Example 93,

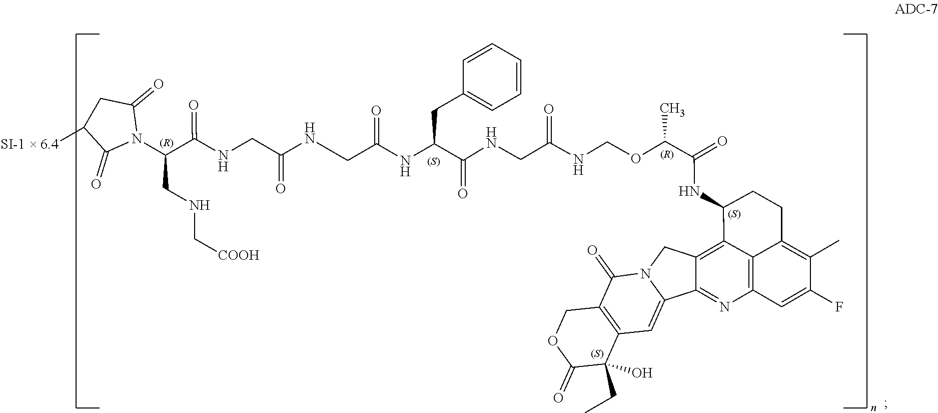

### Example 102

ADC-8 was prepared according to the general-purpose coupling method of Example 93,

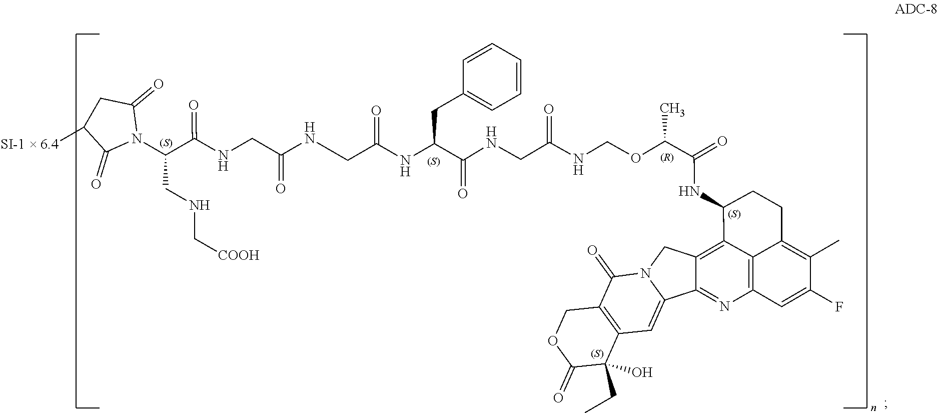

### Example 103

ADC-9 was prepared according to the general-purpose coupling method of Example 93,

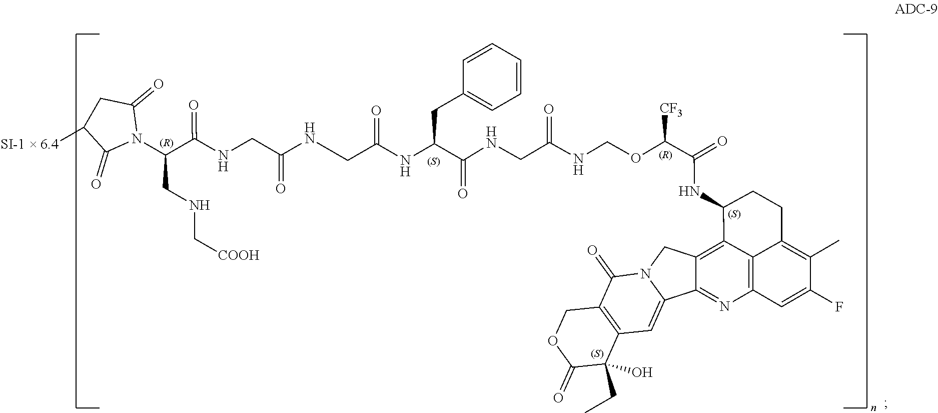

### Example 104

ADC-10 was prepared according to the general-purpose coupling method of Example 93,

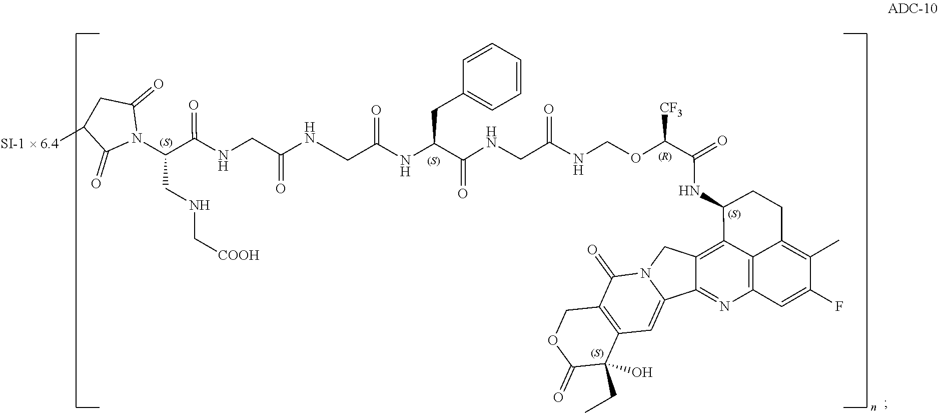

### Example 105

ADC-11 was prepared according to the general-purpose coupling method of Example 93,

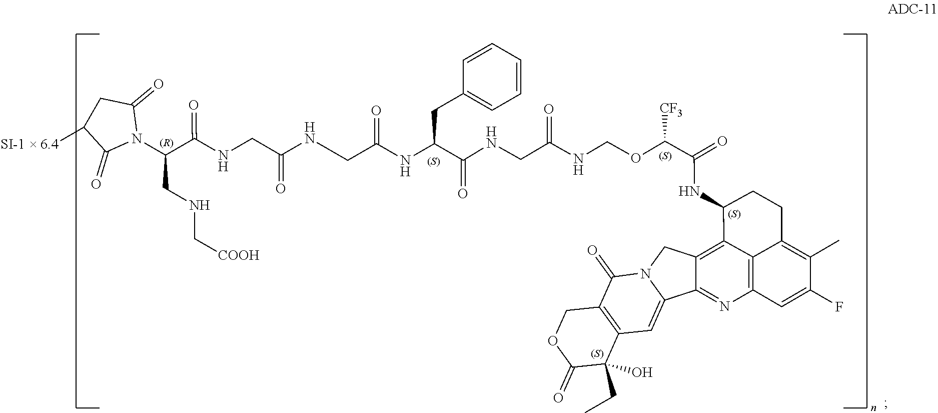

### Example 106

ADC-12 was prepared according to the general-purpose coupling method of Example 93,

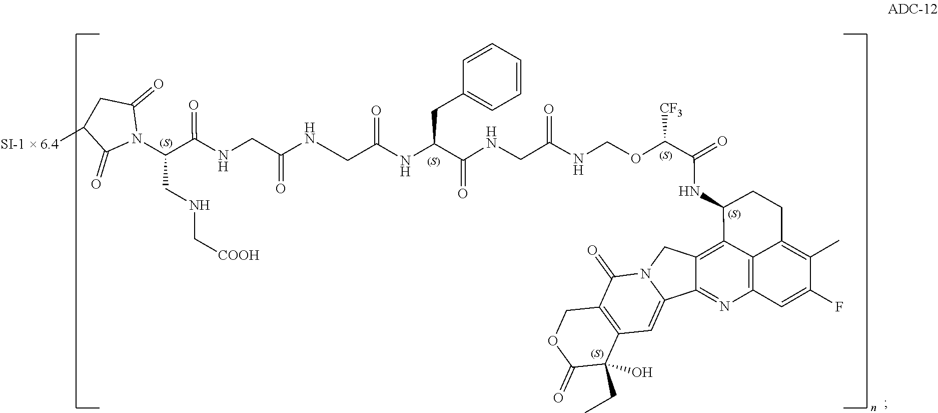

### Example 107

ADC-13 was prepared according to the general-purpose coupling method of Example 93,

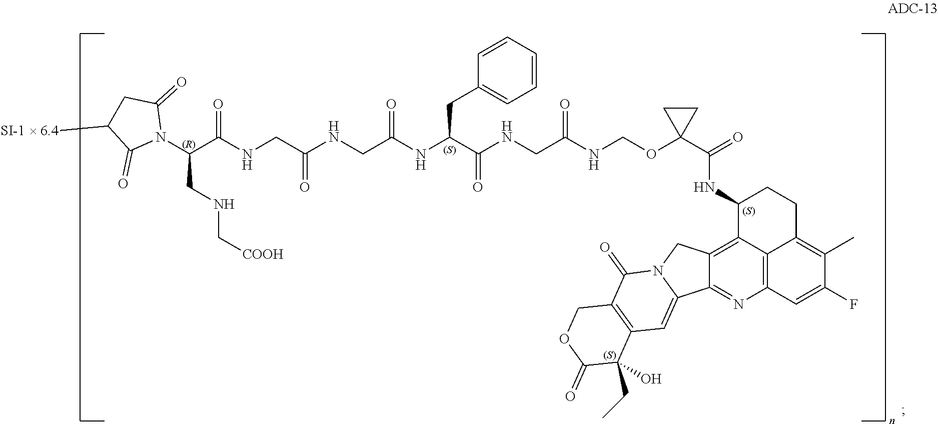

### Example 108

ADC-14 was prepared according to the general-purpose coupling method of Example 93,

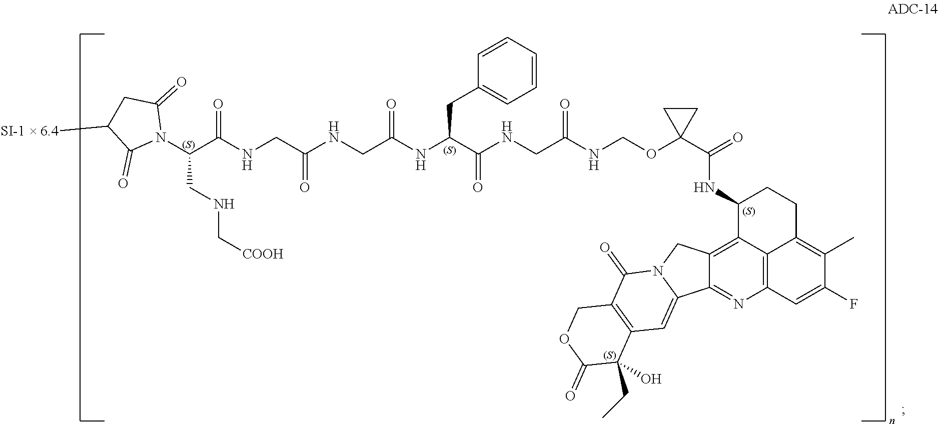

### Example 109

ADC-15 was prepared according to the general-purpose coupling method of Example 93,

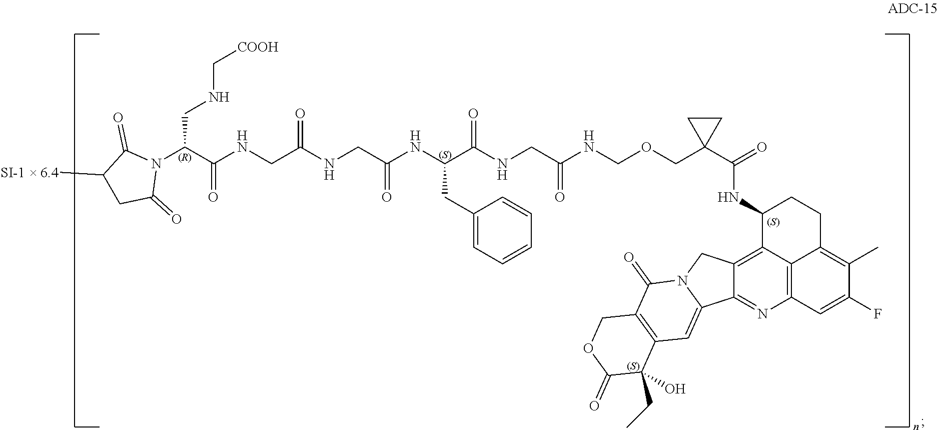

### Example 110

ADC-16 was prepared according to the general-purpose coupling method of Example 93,

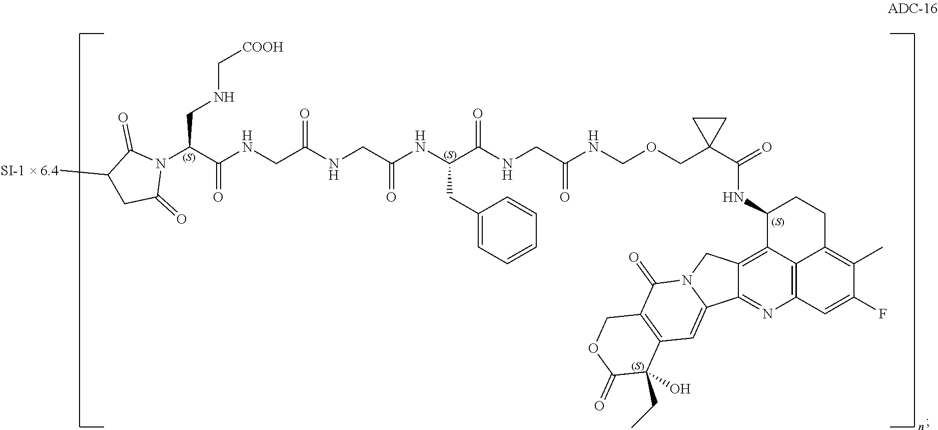

### Example 111

ADC-17 was prepared according to the general-purpose coupling method of Example 93,

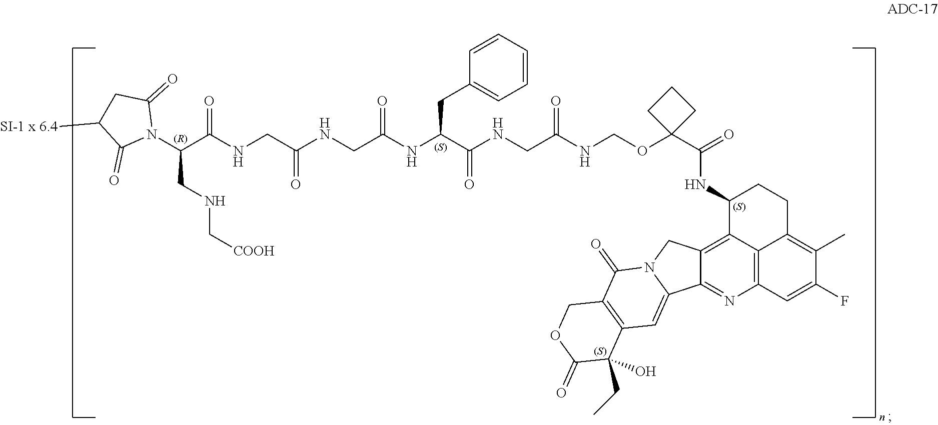

### Example 112

ADC-18 was prepared according to the general-purpose coupling method of Example 93,

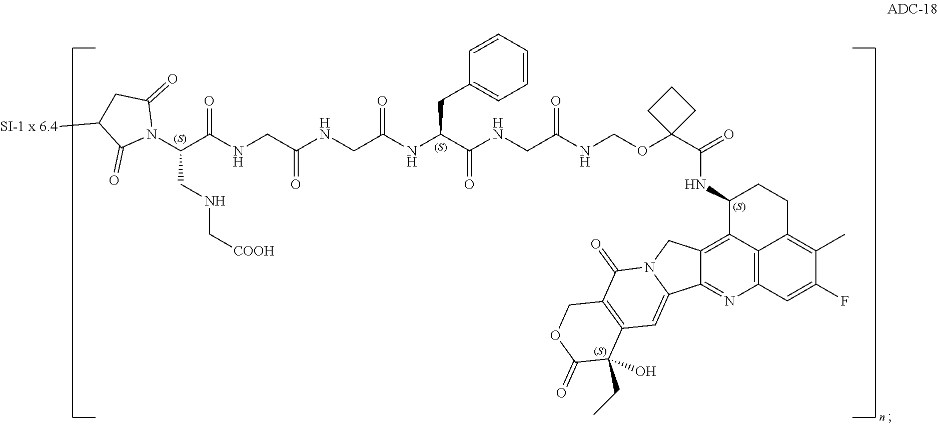

### Example 113

ADC-19 was prepared according to the general-purpose coupling method of Example 93,

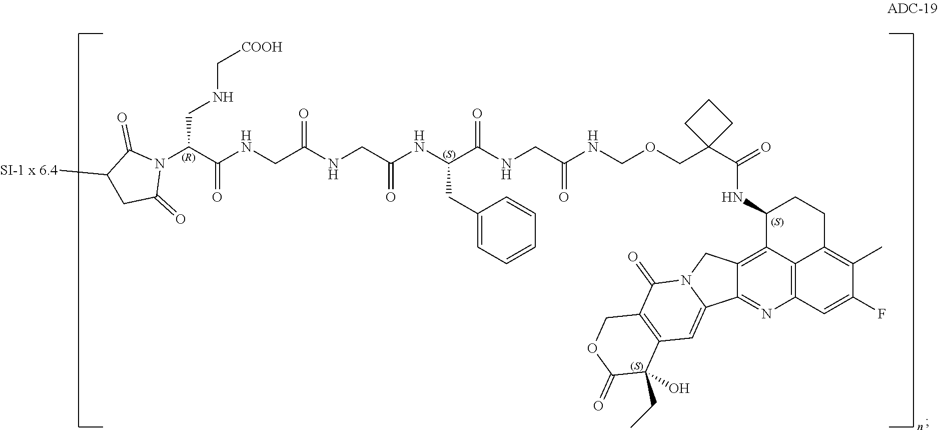

### Example 114

ADC-20 was prepared according to the general-purpose coupling method of Example 93,

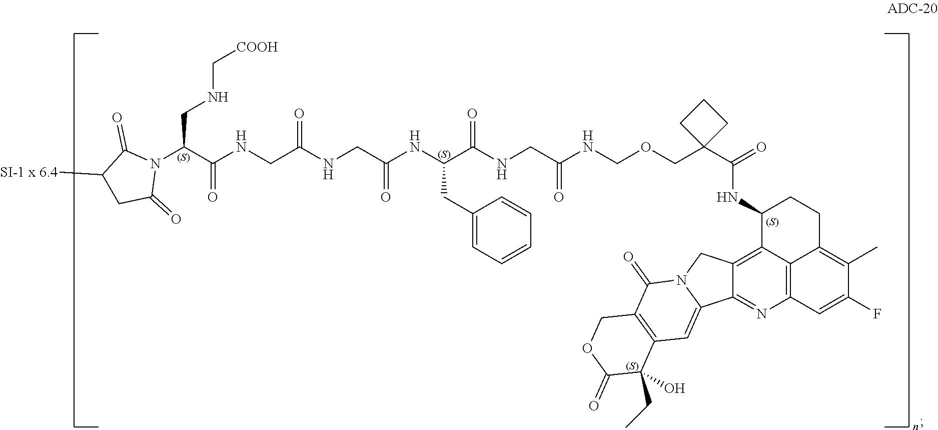

### Example 115

ADC-21 was prepared according to the general-purpose coupling method of Example 93,

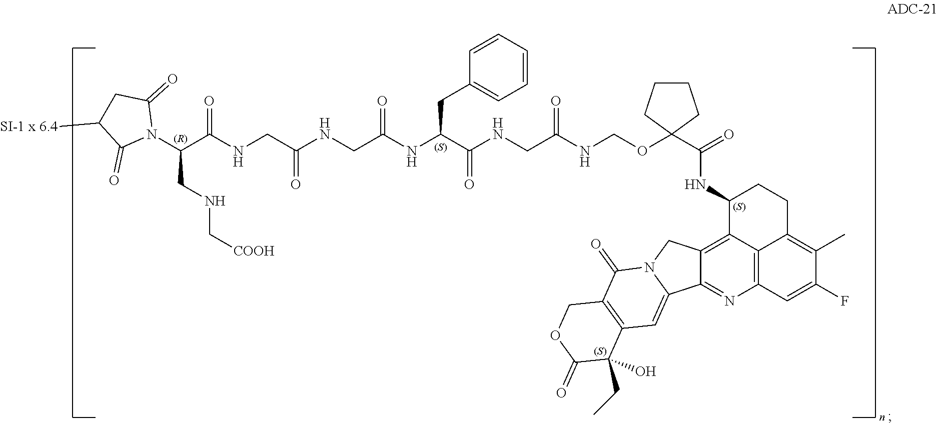

### Example 116

ADC-22 was prepared according to the general-purpose coupling method of Example 93,

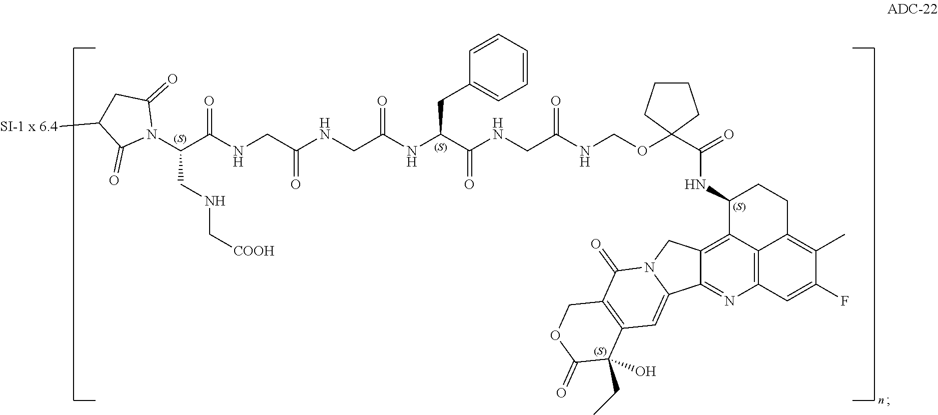

### Example 117

ADC-23 was prepared according to the general-purpose coupling method of Example 93,

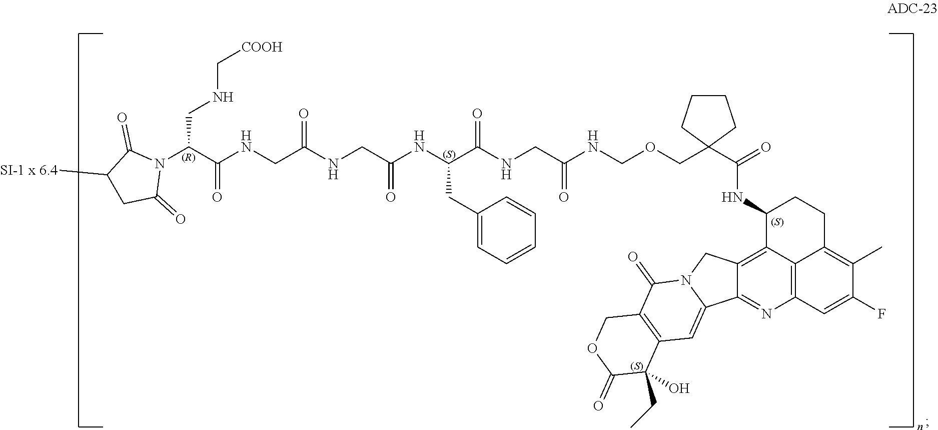

### Example 118

ADC-24 was prepared according to the general-purpose coupling method of Example 93,

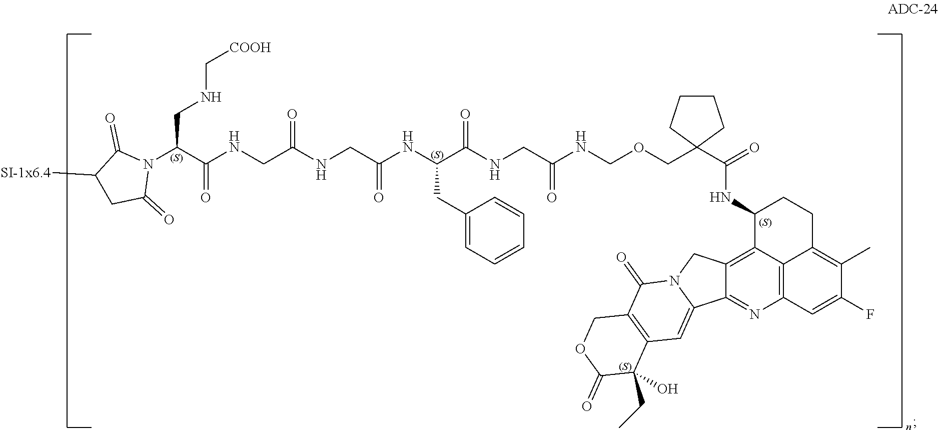

### Example 119

ADC-25 was prepared according to the general-purpose coupling method of Example 93,

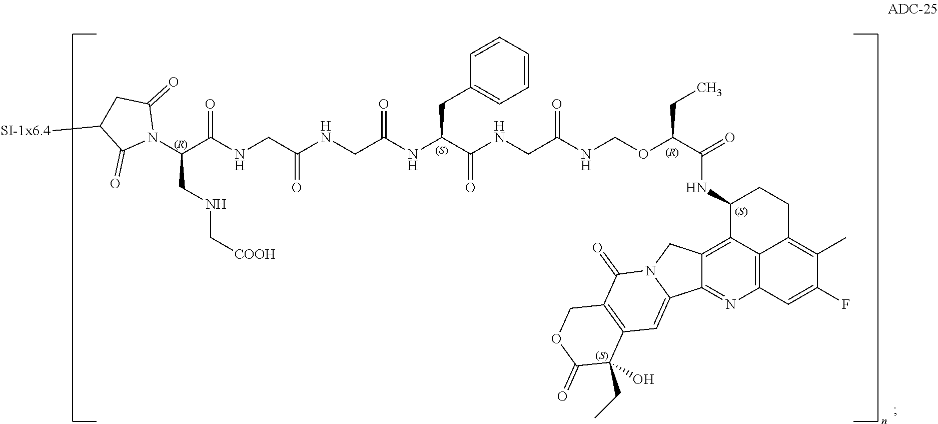

### Example 120

ADC-26 was prepared according to the general-purpose coupling method of Example 93,

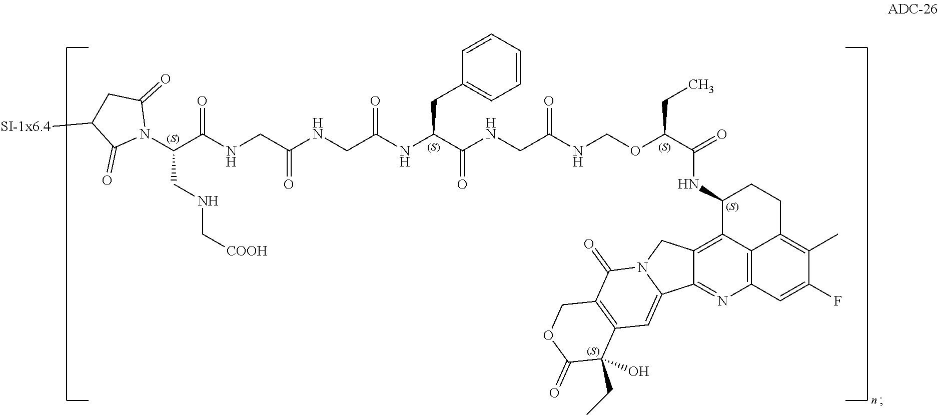

### Example 121

ADC-27 was prepared according to the general-purpose coupling method of Example 93,v

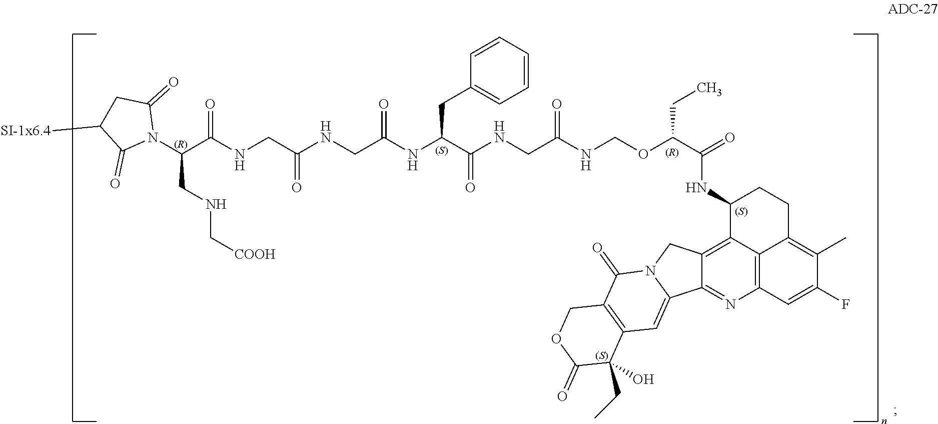

### Example 122

ADC-28 was prepared according to the general-purpose coupling method of Example 93,

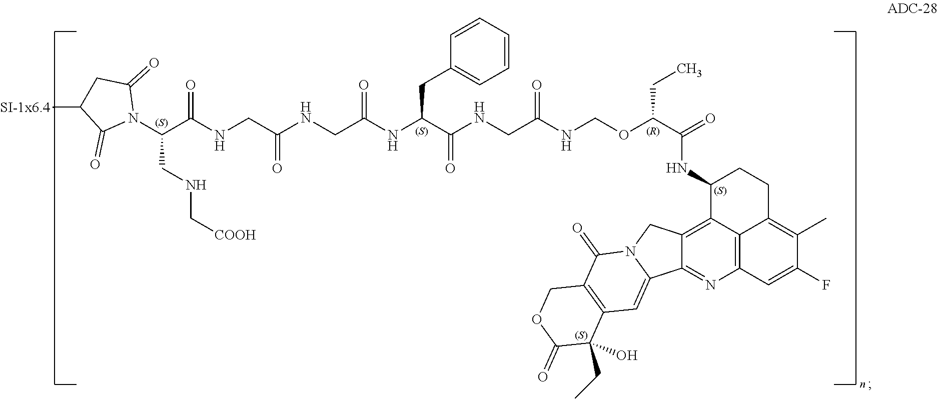

### Example 123

ADC-29 was prepared according to the general-purpose coupling method of Example 93,

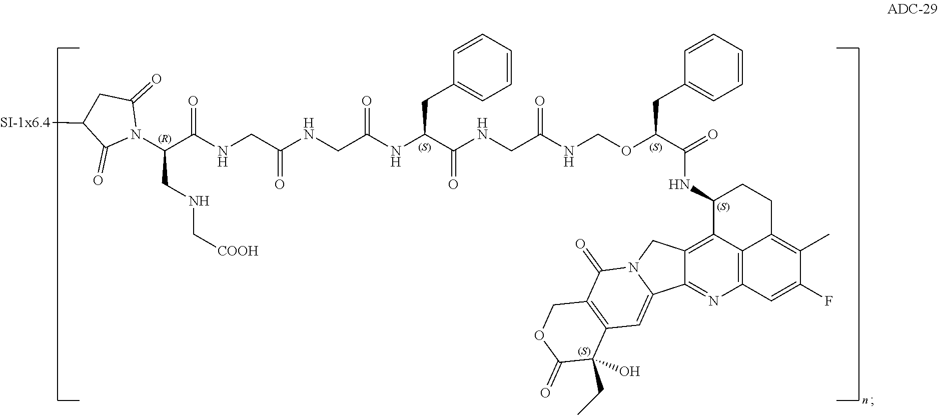

### Example 124

ADC-30 was prepared according to the general-purpose coupling method of Example 93,

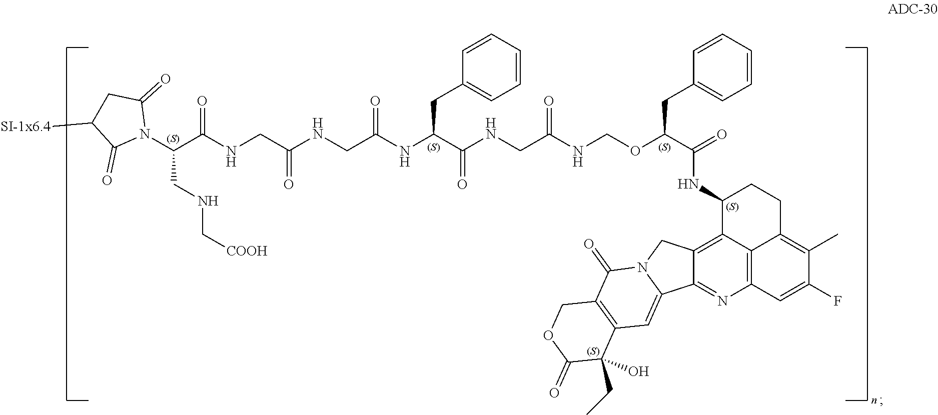

### Example 125

ADC-31 was prepared according to the general-purpose coupling method of Example 93,

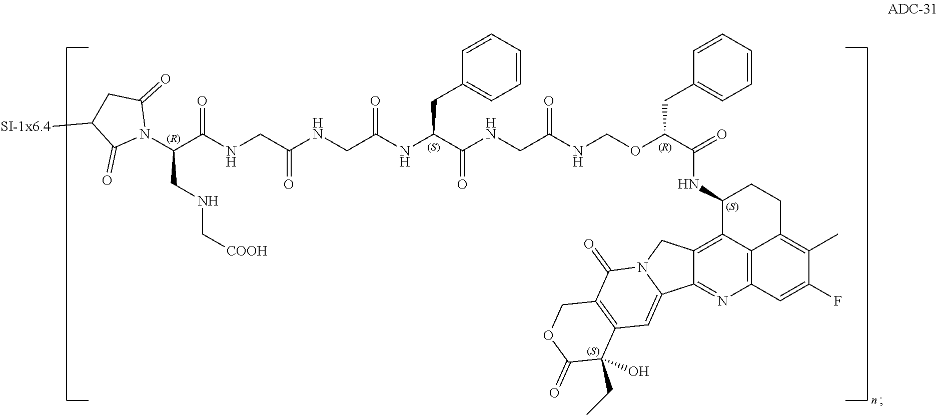

### Example 126

ADC-32 was prepared according to the general-purpose coupling method of Example 93,

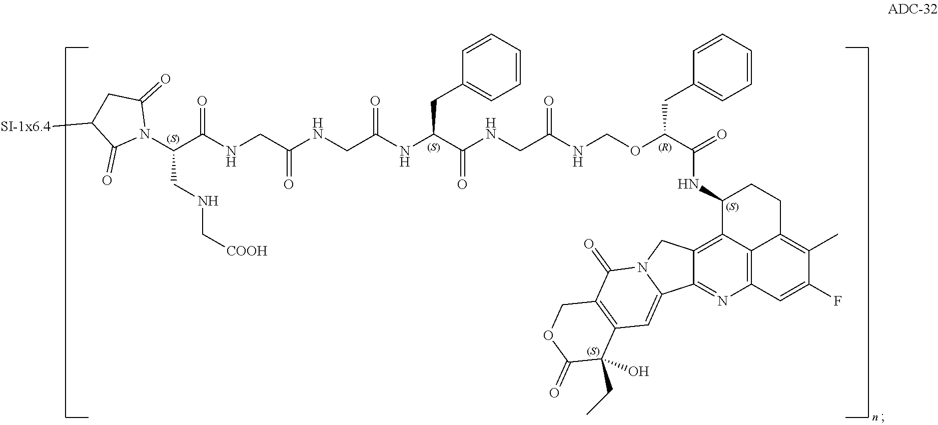

### Example 127

ADC-33 was prepared according to the general-purpose coupling method of Example 93,

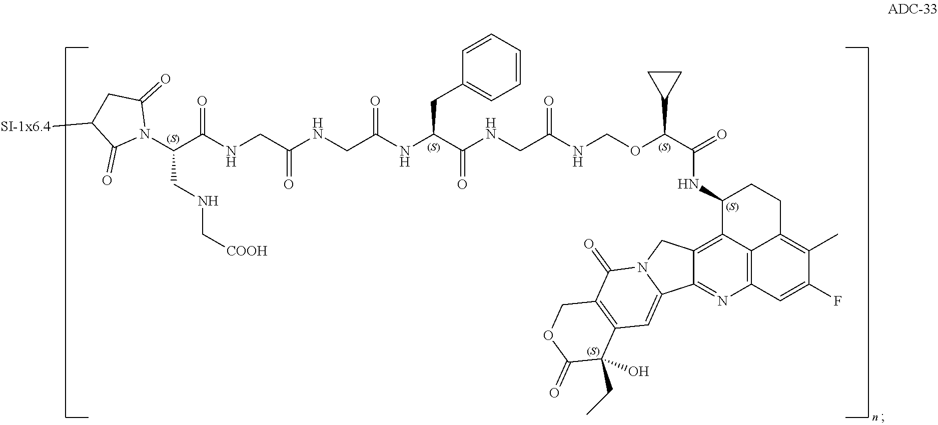

### Example 128

ADC-34 was prepared according to the general-purpose coupling method of Example 93,

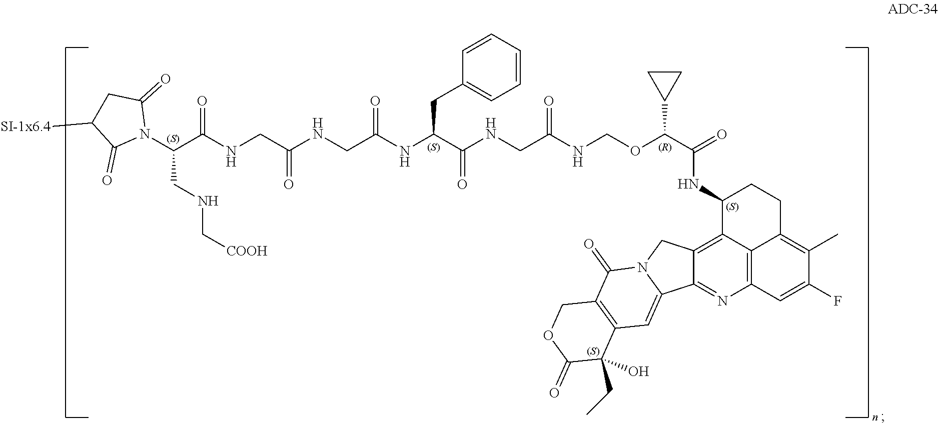

### Example 129

ADC-35 was prepared according to the general-purpose coupling method of Example 93,

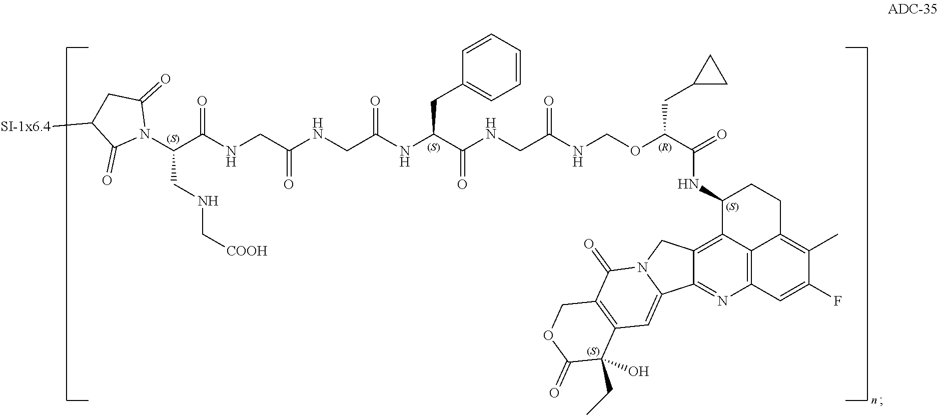

### Example 130

ADC-36 was prepared according to the general-purpose coupling method of Example 93,

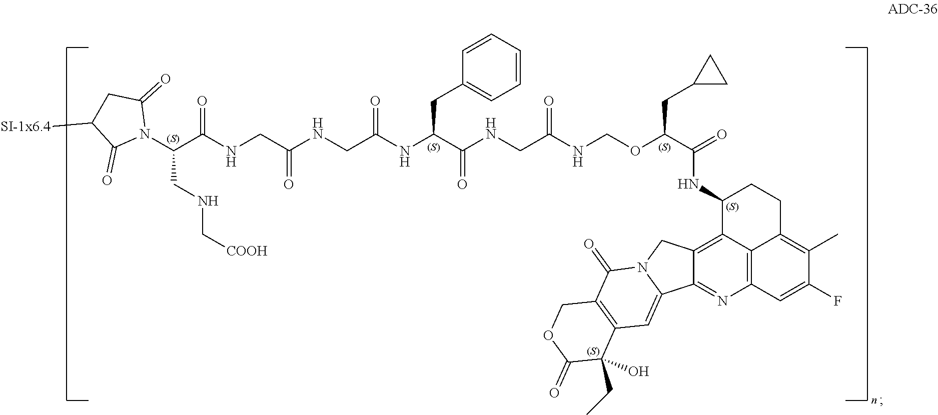

### Example 131

ADC-37 was prepared according to the general-purpose coupling method of Example 93,

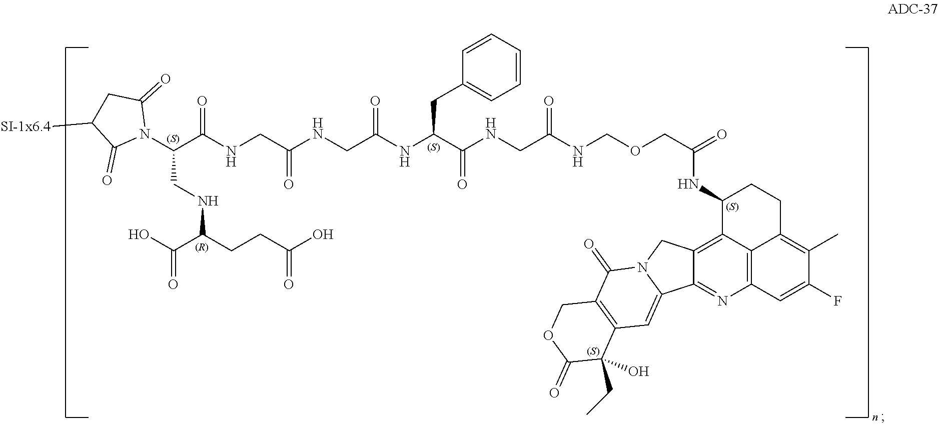

### Example 132

ADC-38 was prepared according to the general-purpose coupling method of Example 93,

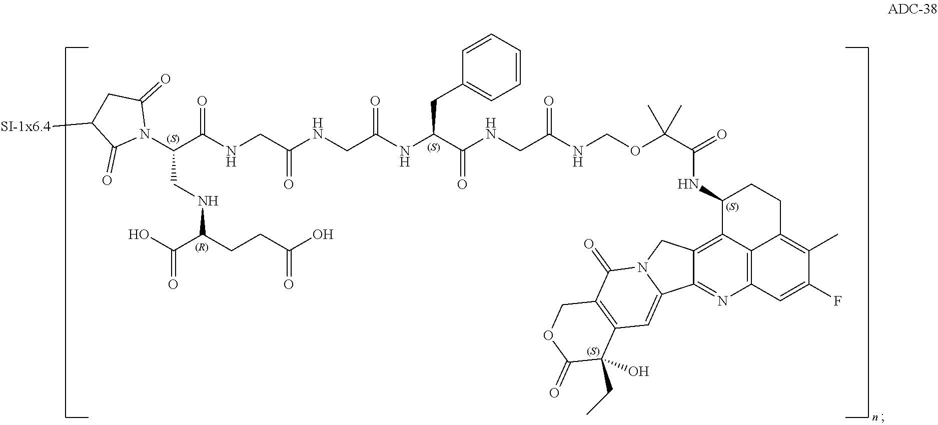

### Example 133

ADC-39 was prepared according to the general-purpose coupling method of Example 93,

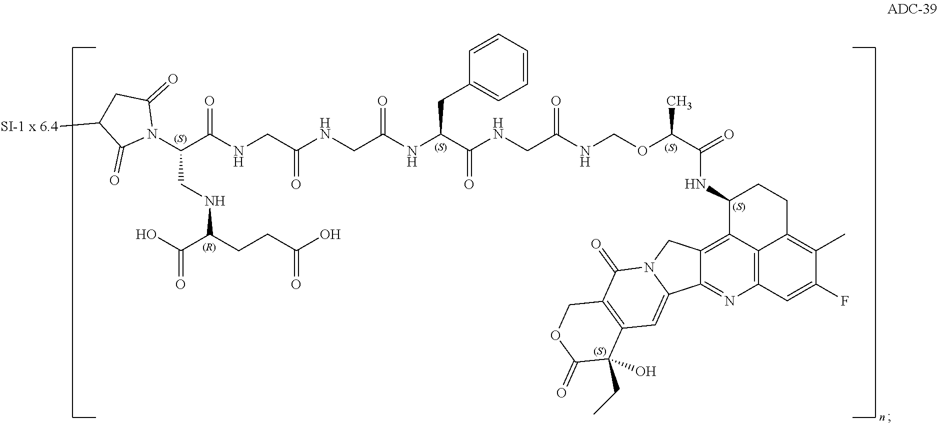

### Example 134

ADC-40 was prepared according to the general-purpose coupling method of Example 93,

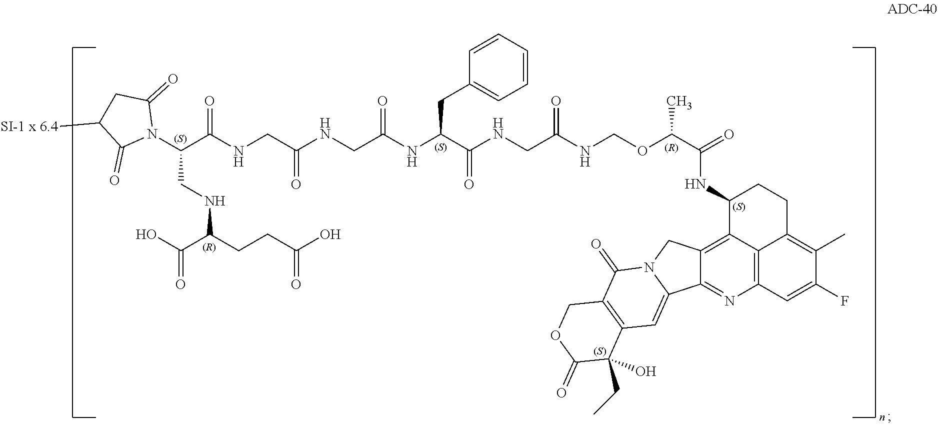

### Example 135

ADC-41 was prepared according to the general-purpose coupling method of Example 93,

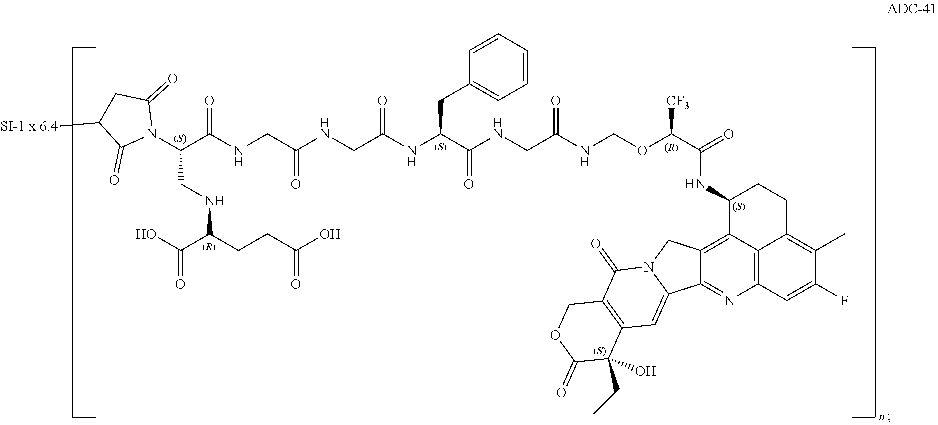

### Example 136

ADC-42 was prepared according to the general-purpose coupling method of Example 93,

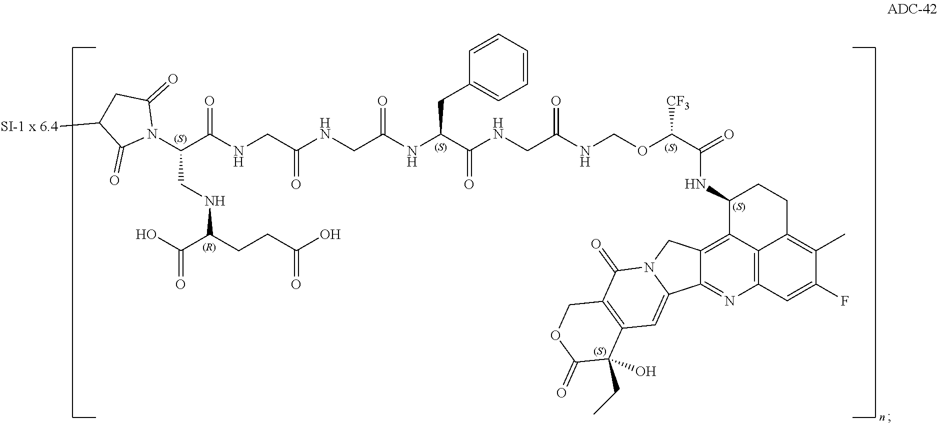

### Example 137

ADC-43 was prepared according to the general-purpose coupling method of Example 93,

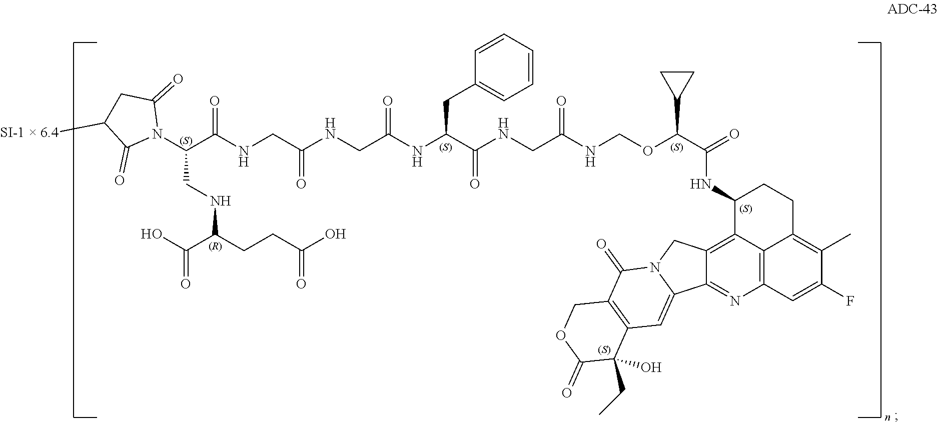

### Example 138

ADC-44 was prepared according to the general-purpose coupling method of Example 93,

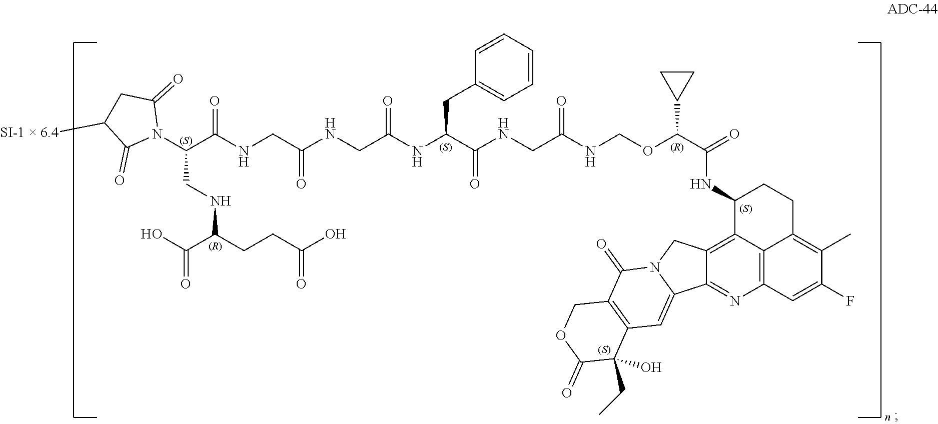

### Example 139

ADC-45 was prepared according to the general-purpose coupling method of Example 93,

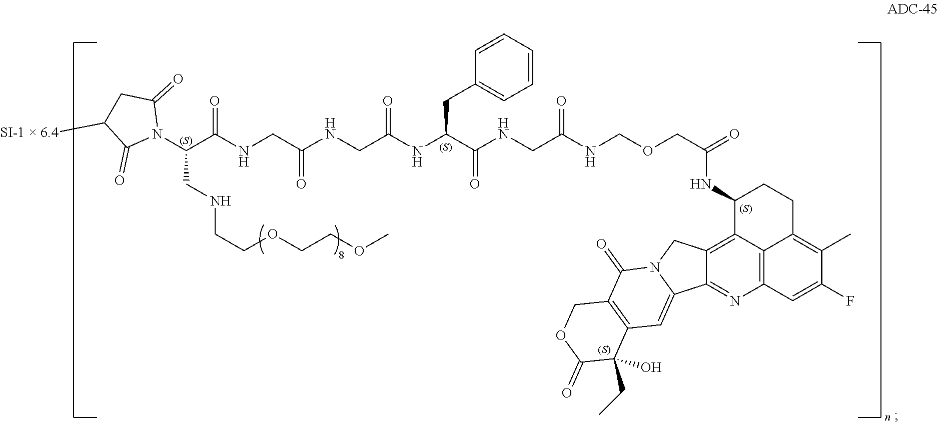

### Example 140

ADC-46 was prepared according to the general-purpose coupling method of Example 93,

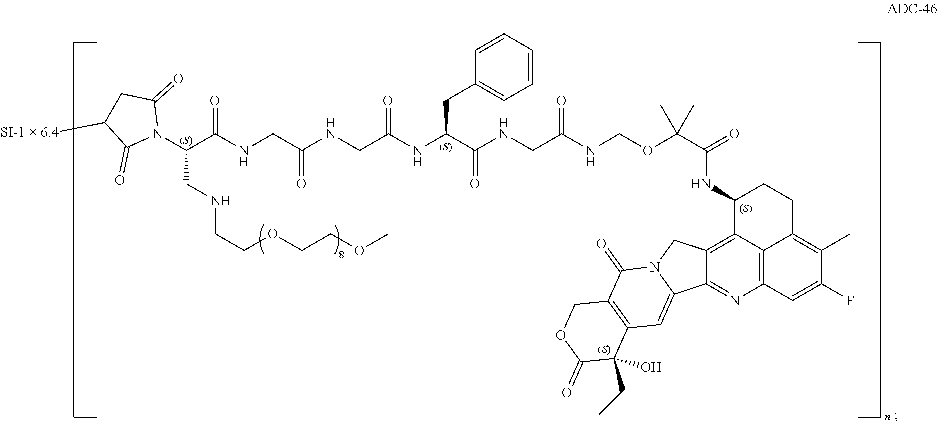

### Example 141

ADC-47 was prepared according to the general-purpose coupling method of Example 93,

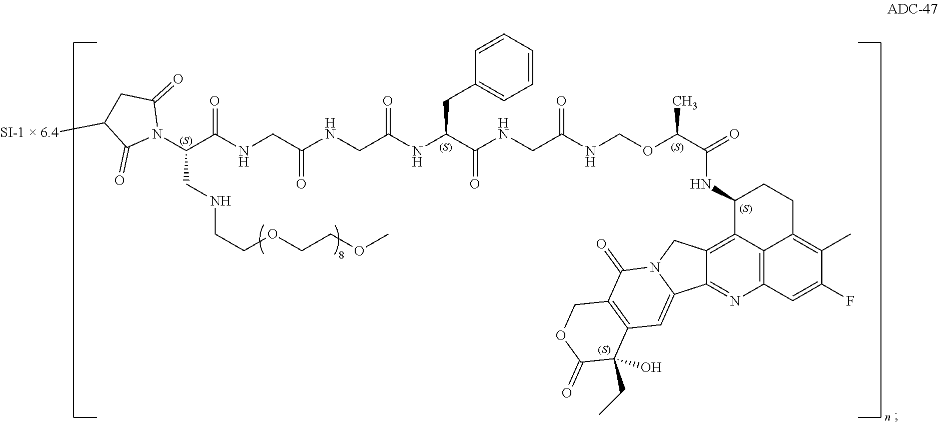

### Example 142

ADC-48 was prepared according to the general-purpose coupling method of Example 93,

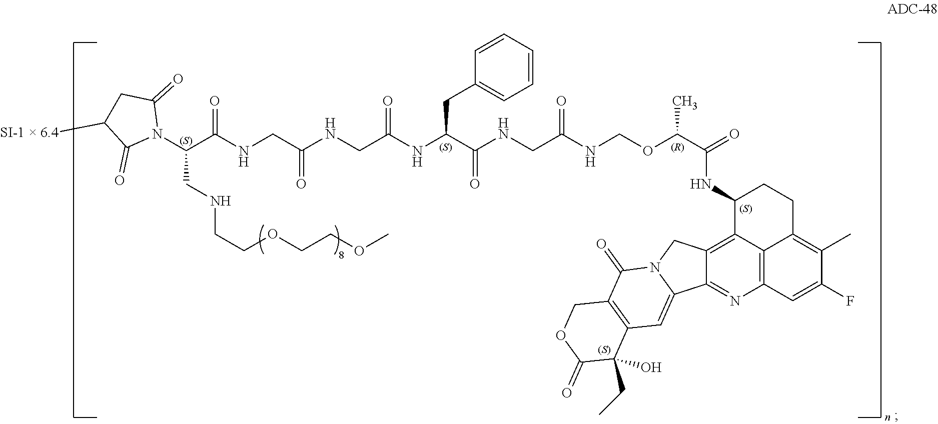

### Example 143

ADC-49 was prepared according to the general-purpose coupling method of Example 93,

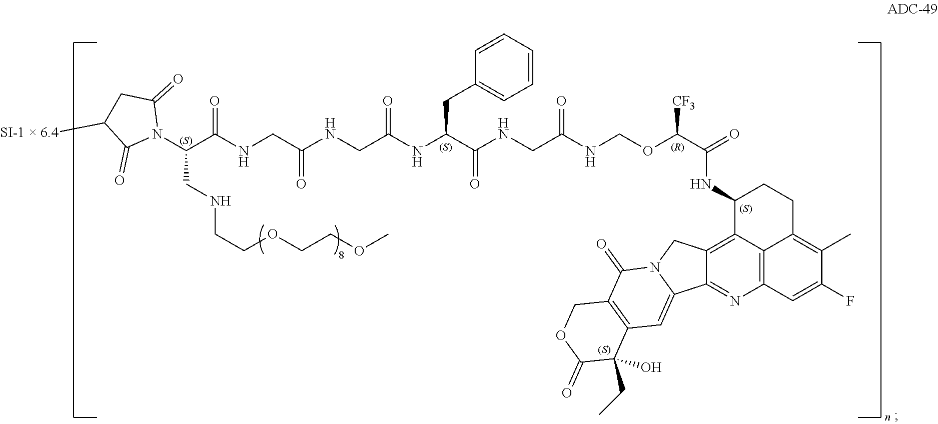

### Example 144

ADC-50 was prepared according to the general-purpose coupling method of Example 93,

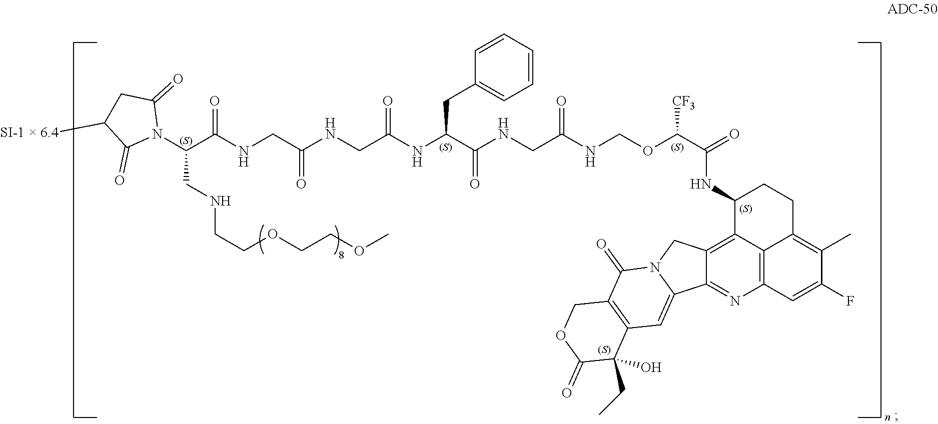

### Example 145

ADC-51 was prepared according to the general-purpose coupling method of Example 93,

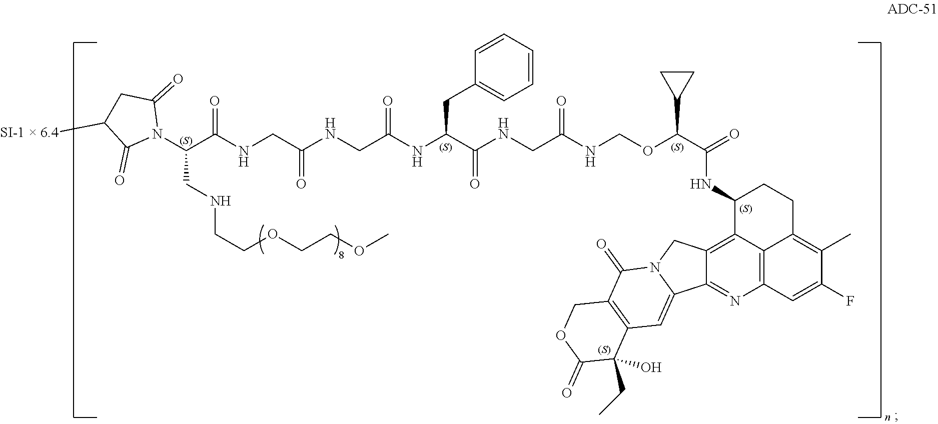

### Example 146

ADC-52 was prepared according to the general-purpose coupling method of Example 93,

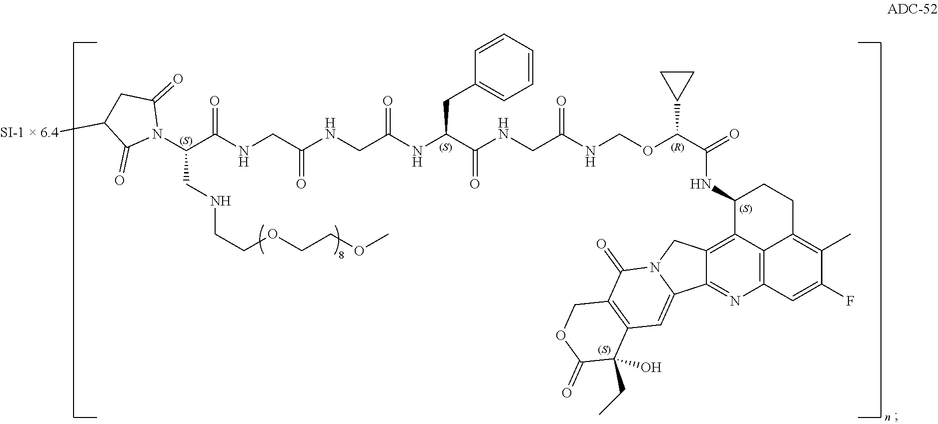

### Example 147

ADC-53 was prepared according to the general-purpose coupling method of Example 93,

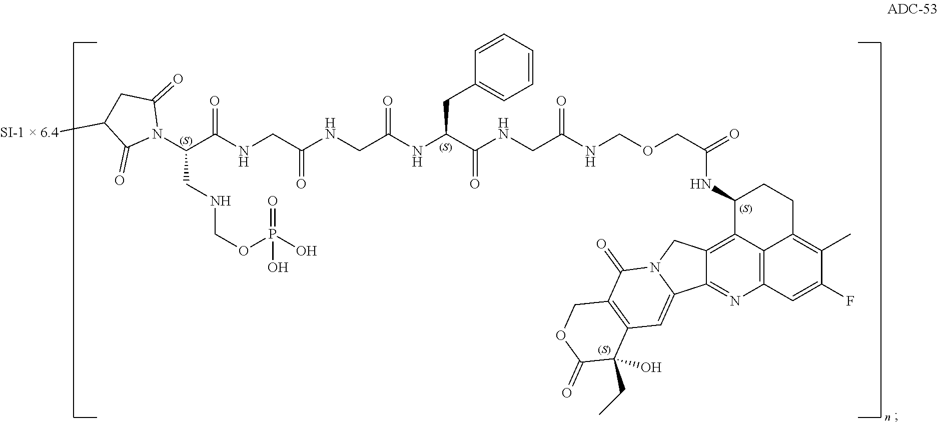

### Example 148

ADC-54 was prepared according to the general-purpose coupling method of Example 93,

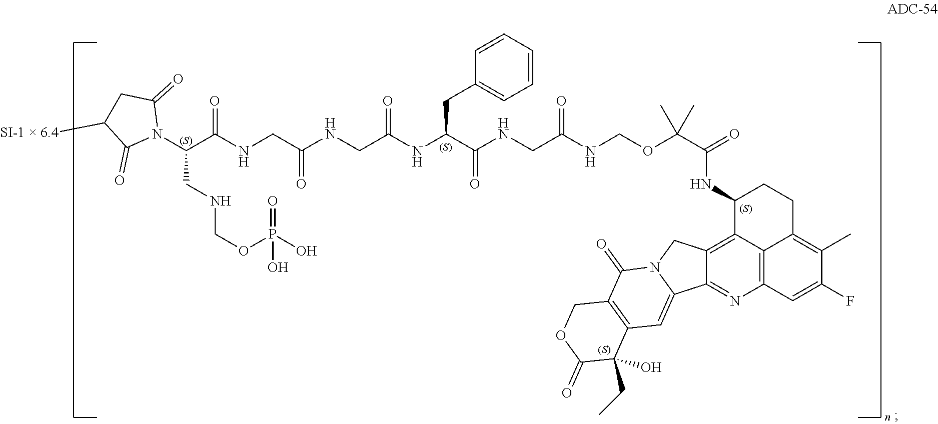

### Example 149

ADC-55 was prepared according to the general-purpose coupling method of Example 93,

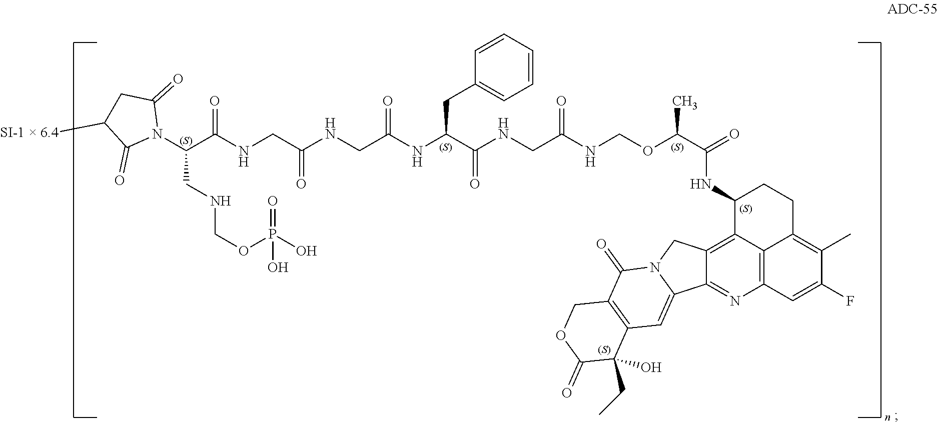

### Example 150

ADC-56 was prepared according to the general-purpose coupling method of Example 93,

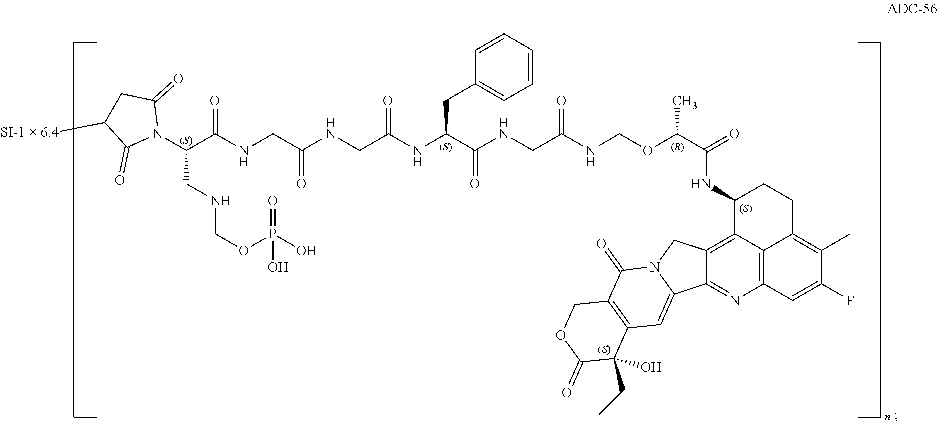

### Example 151

ADC-57 was prepared according to the general-purpose coupling method of Example 93,

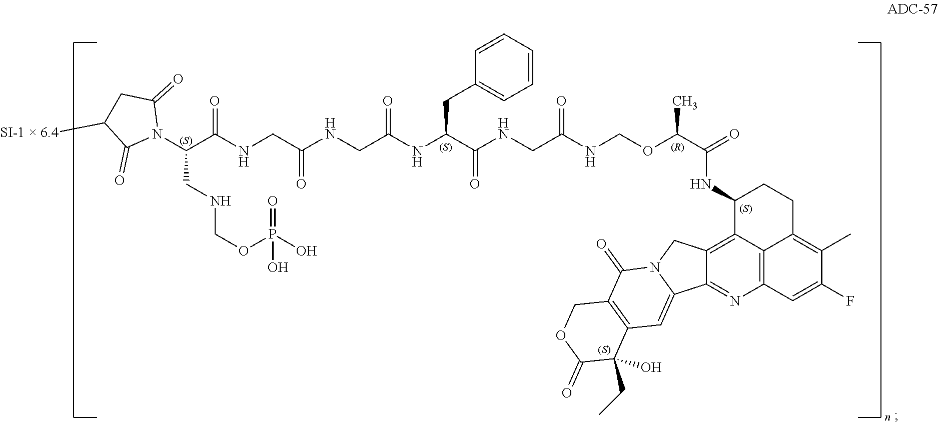

### Example 152

ADC-58 was prepared according to the general-purpose coupling method of Example 93,

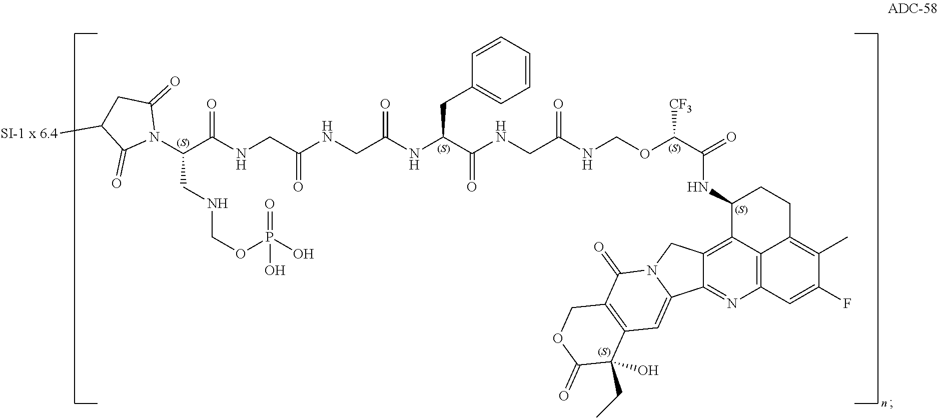

### Example 153

ADC-59 was prepared according to the general-purpose coupling method of Example 93,

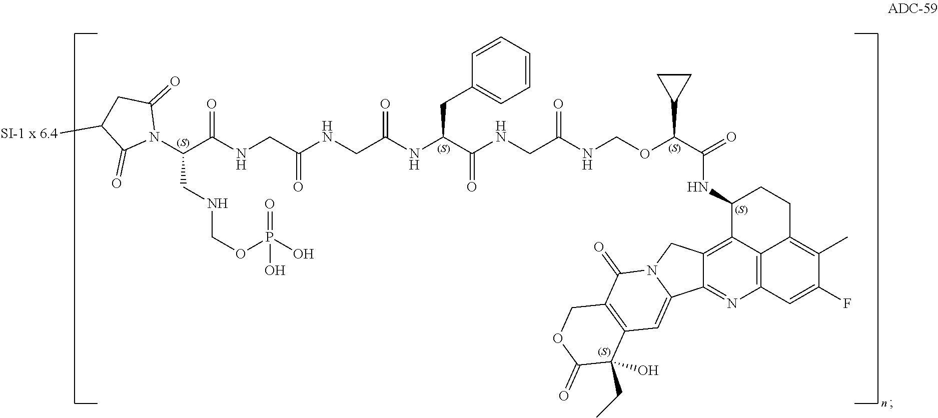

### Example 154

ADC-60 was prepared according to the general-purpose coupling method of Example 93,

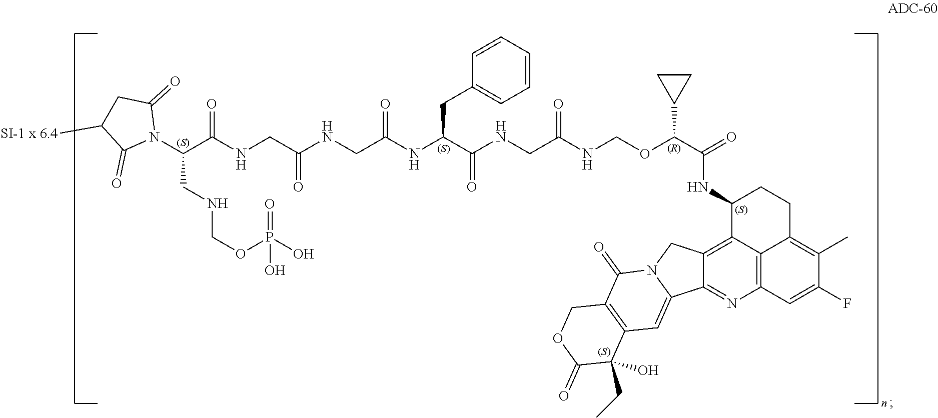

### Example 155

ADC-61 was prepared according to the general-purpose coupling method of Example 93,

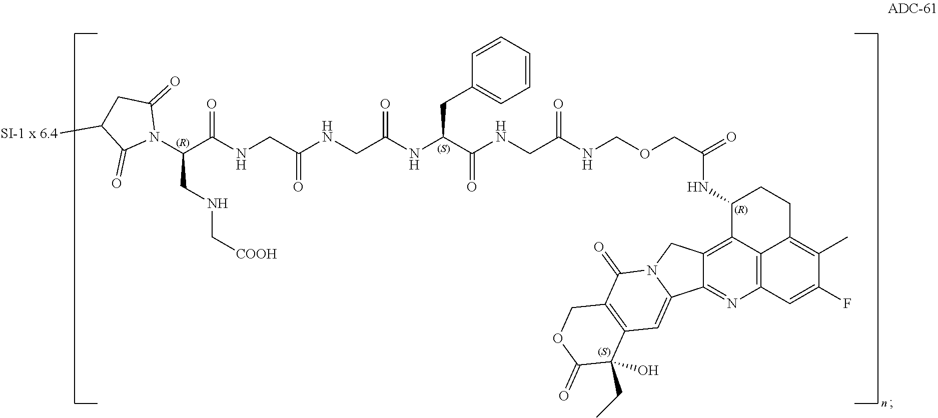

### Example 156

ADC-62 was prepared according to the general-purpose coupling method of Example 93,

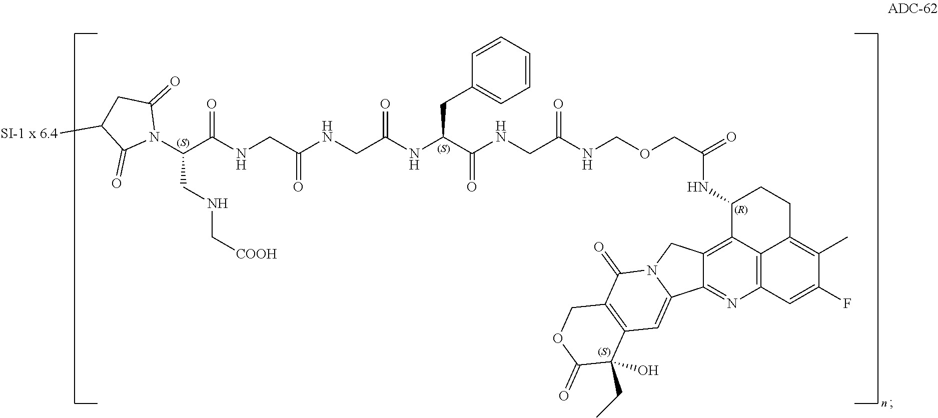

### Example 157

ADC-63 was prepared according to the general-purpose coupling method of Example 93,

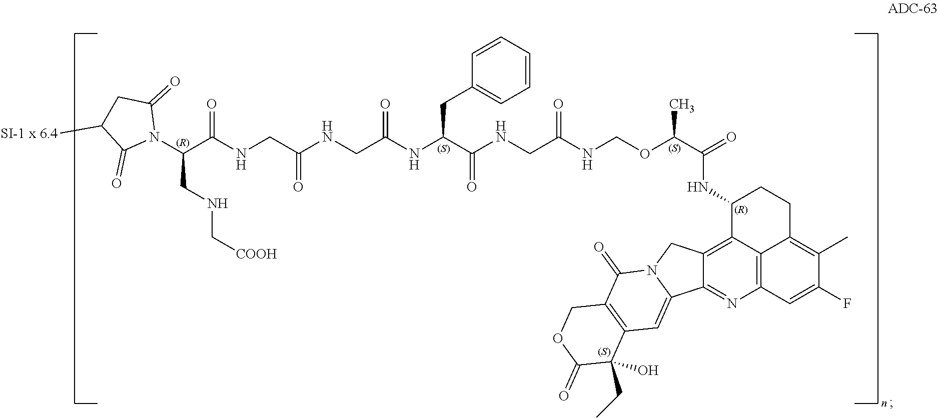

### Example 158

ADC-64 was prepared according to the general-purpose coupling method of Example 93,

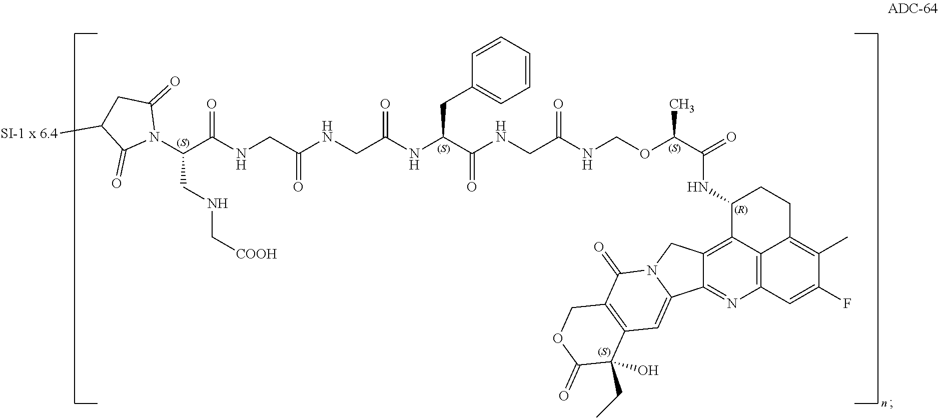

### Example 159

ADC-65 was prepared according to the general-purpose coupling method of Example 93,

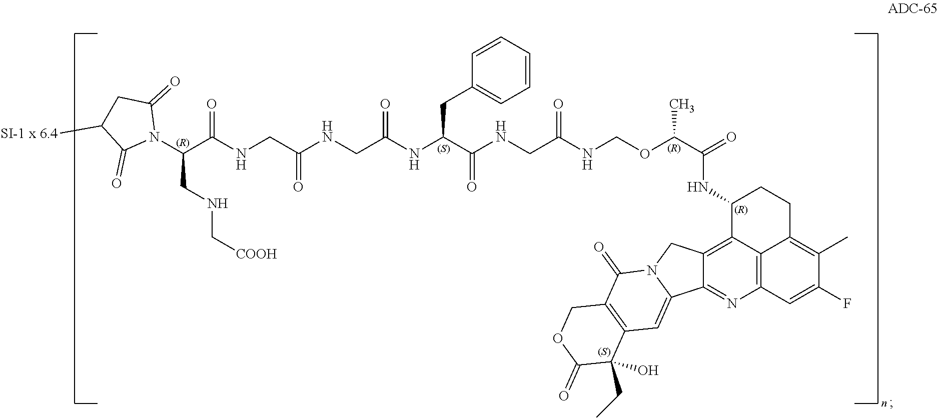

### Example 160

ADC-66 was prepared according to the general-purpose coupling method of Example 93,

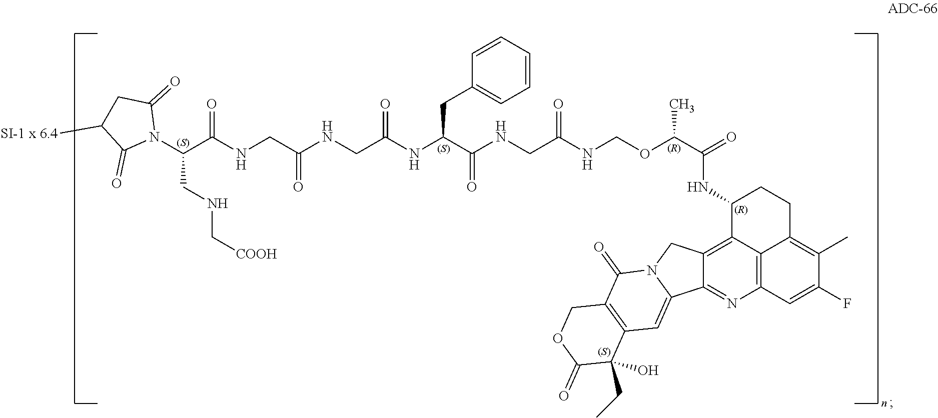

### Example 161

ADC-67 was prepared according to the general-purpose coupling method of Example 93,

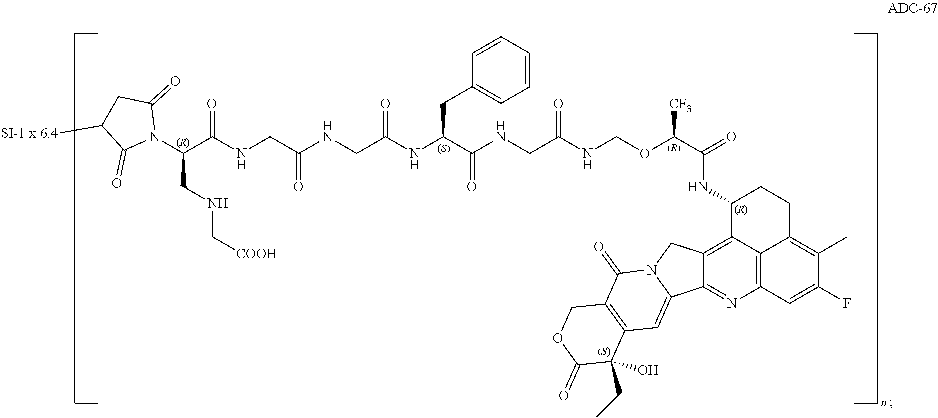

### Example 162

ADC-68 was prepared according to the general-purpose coupling method of Example 93,

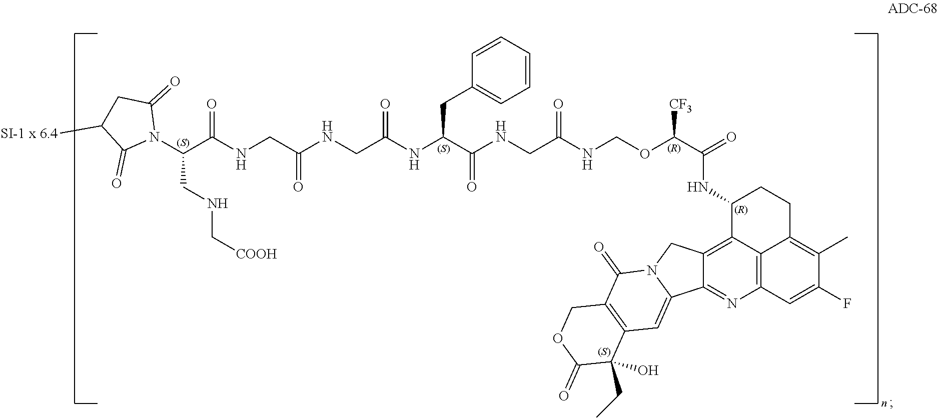

### Example 163

ADC-69 was prepared according to the general-purpose coupling method of Example 93,

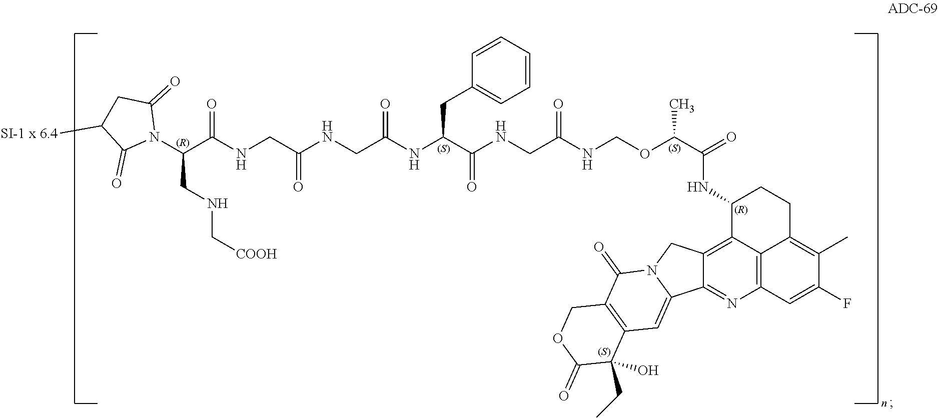

### Example 164

ADC-70 was prepared according to the general-purpose coupling method of Example 93,

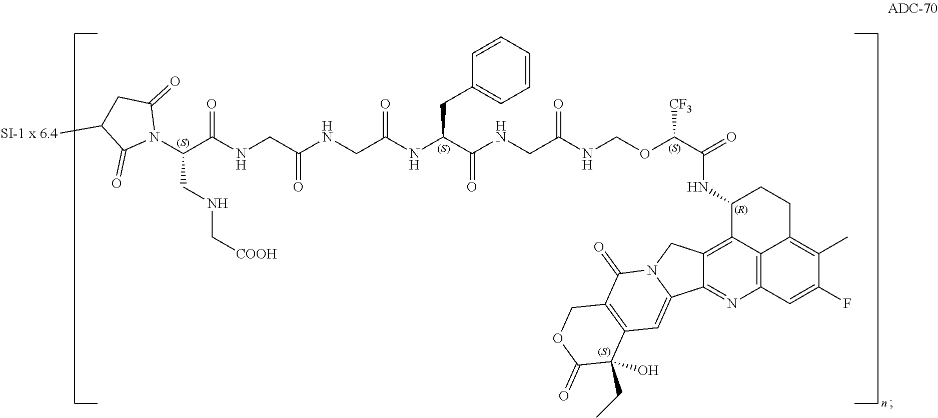

### Example 165

ADC-71 was prepared according to the general-purpose coupling method of Example 93,

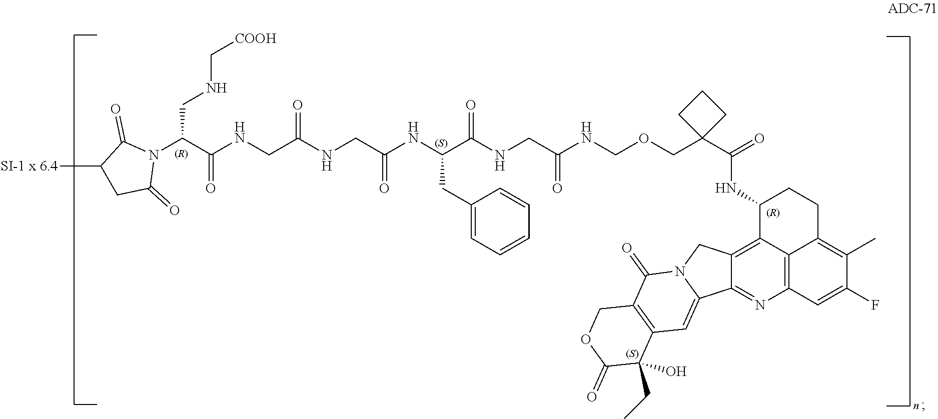

### Example 166

ADC-72 was prepared according to the general-purpose coupling method of Example 93,

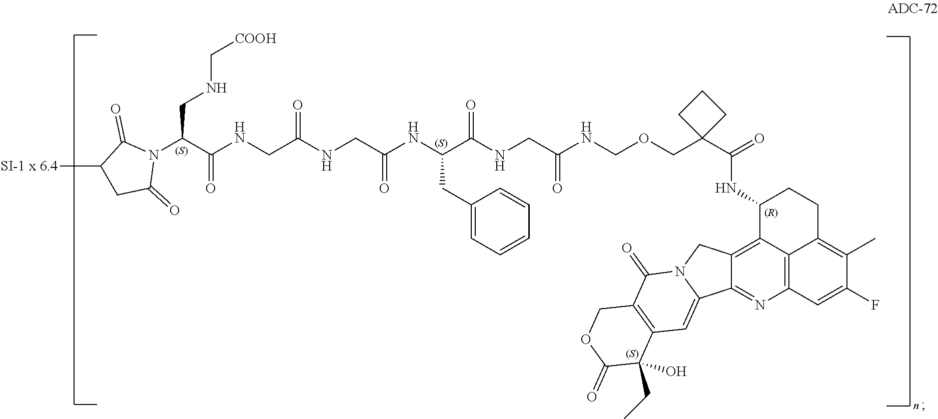

### Example 167

ADC-73 was prepared according to the general-purpose coupling method of Example 93,

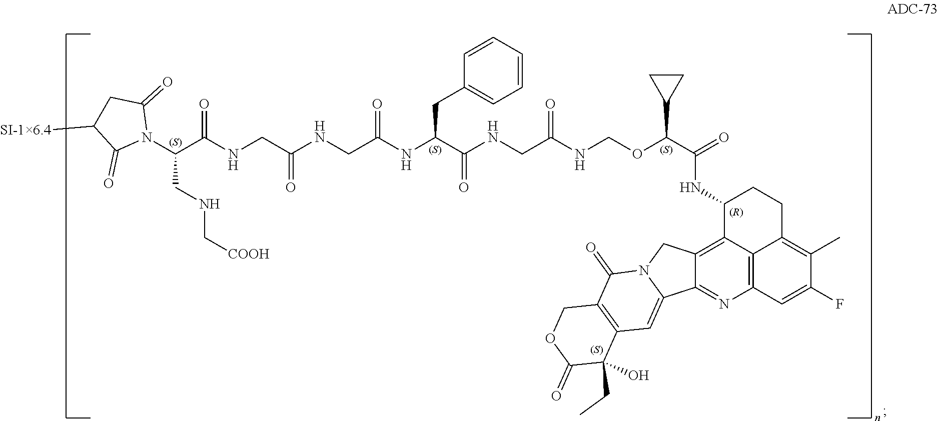

### Example 168

ADC-74 was prepared according to the general-purpose coupling method of Example 93,

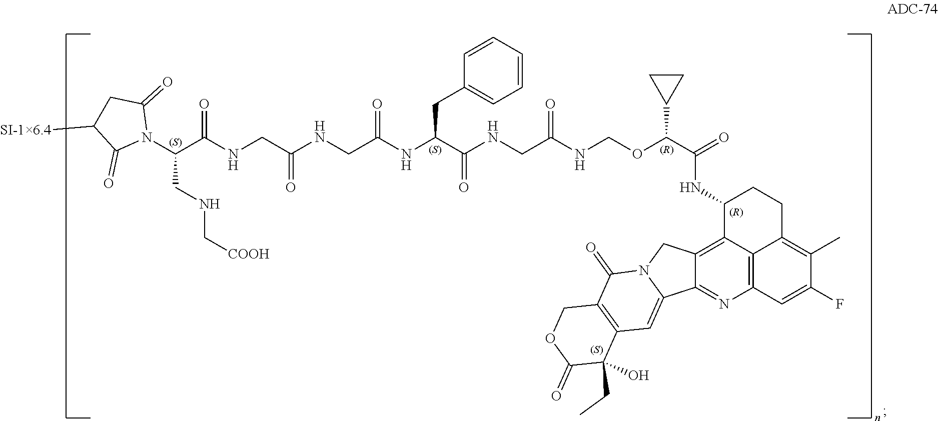

### Example 169

ADC-75 was prepared according to the general-purpose coupling method of Example 93,

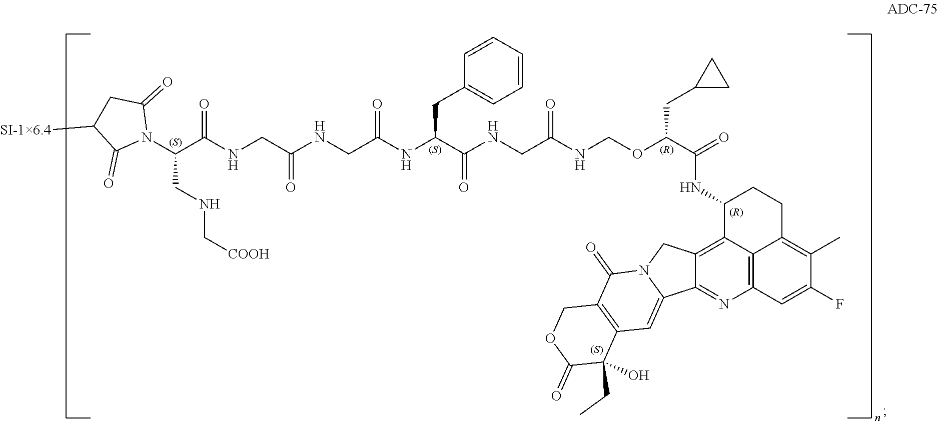

### Example 170

ADC-76 was prepared according to the general-purpose coupling method of Example 93,

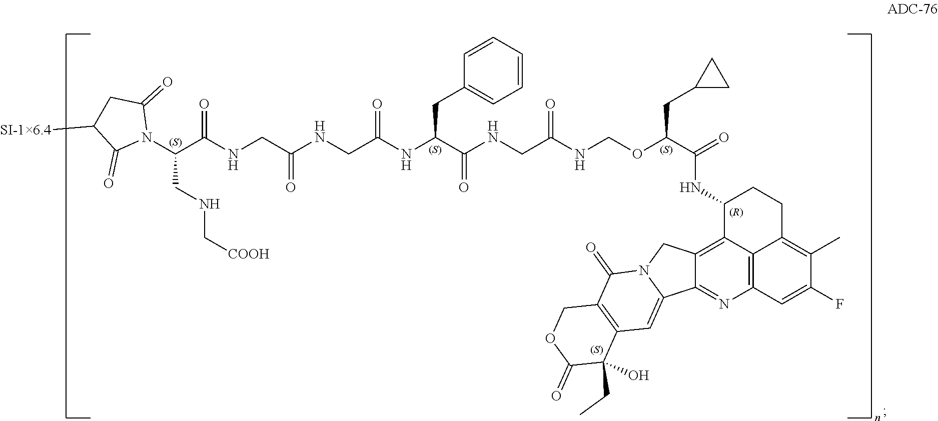

### Example 171

ADC-77 was prepared according to the general-purpose coupling method of Example 93,

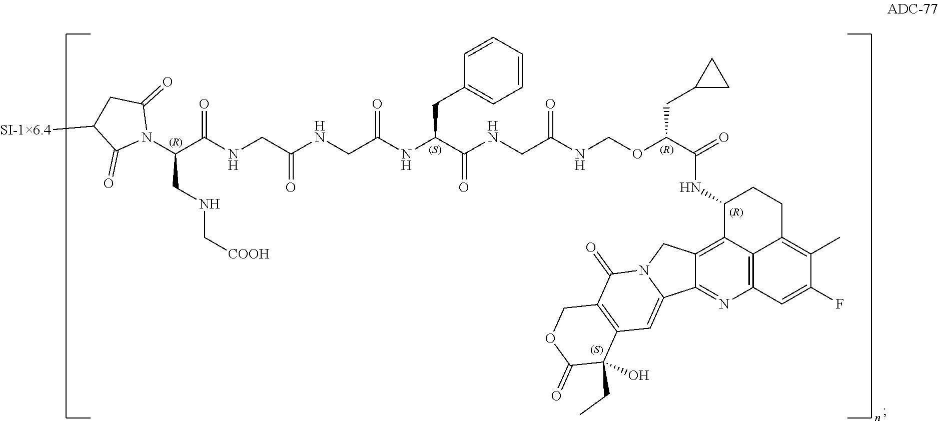

### Example 172

ADC-78 was prepared according to the general-purpose coupling method of Example 93,

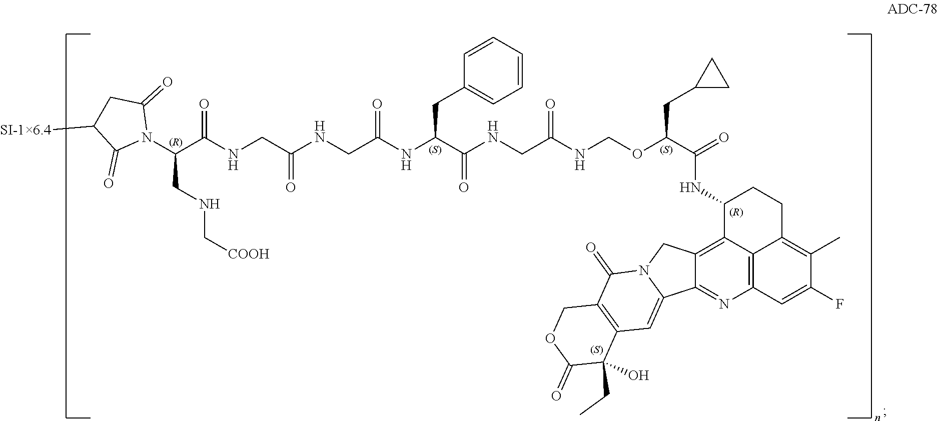

### Example 173

ADC-79 was prepared according to the general-purpose coupling method of Example 93,

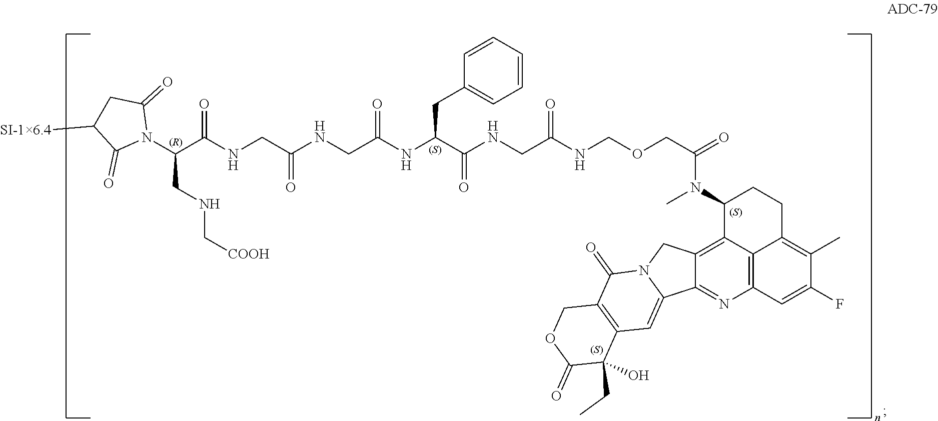

### Example 174

ADC-80 was prepared according to the general-purpose coupling method of Example 93,

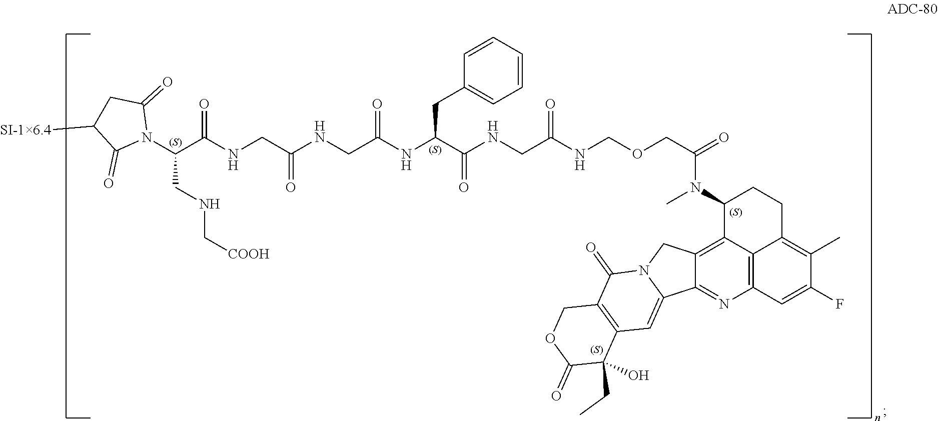

### Example 175

ADC-81 was prepared according to the general-purpose coupling method of Example 93,

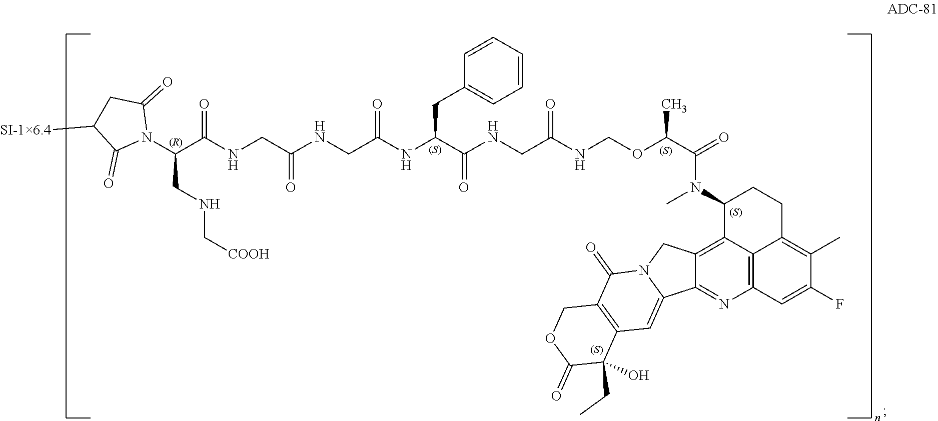

### Example 176

ADC-82 was prepared according to the general-purpose coupling method of Example 93,

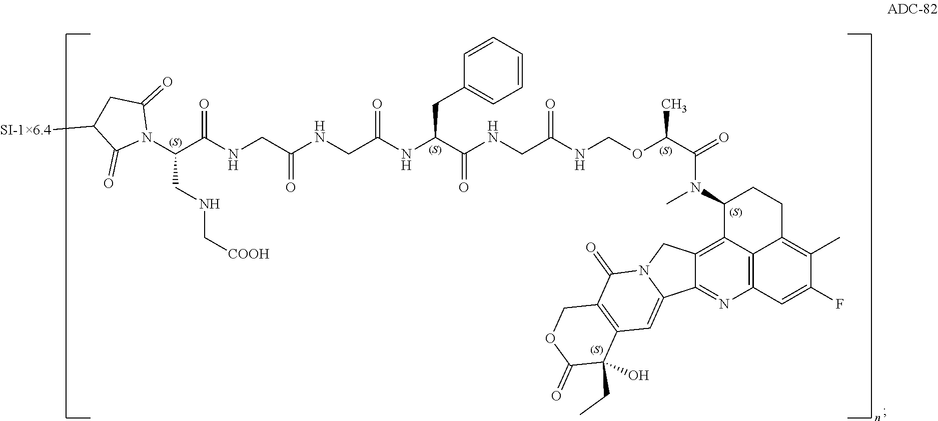

### Example 177

ADC-83 was prepared according to the general-purpose coupling method of Example 93,

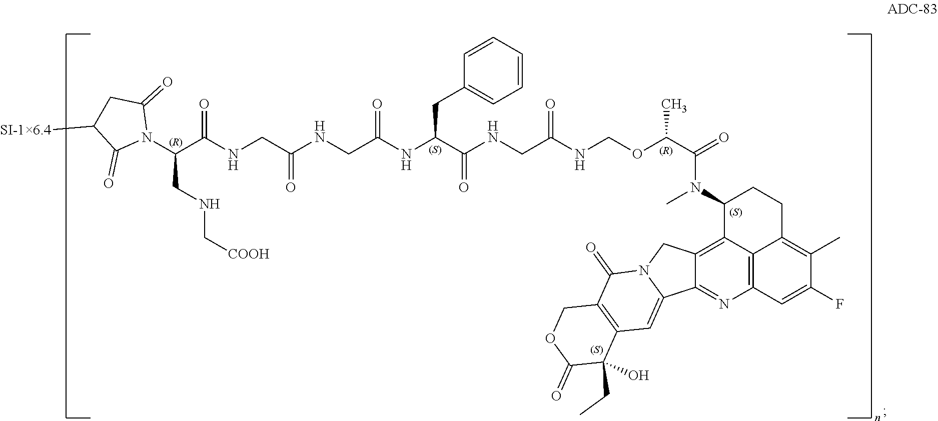

### Example 178

ADC-84 was prepared according to the general-purpose coupling method of Example 93,

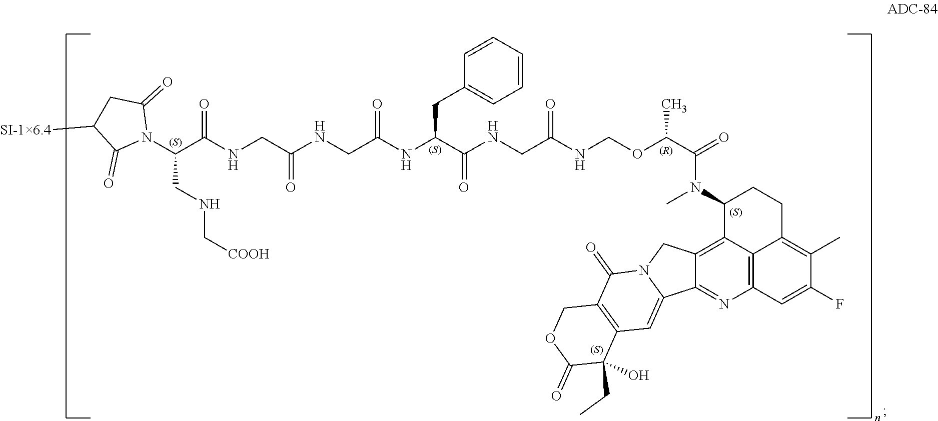

### Example 179

ADC-85 was prepared according to the general-purpose coupling method of Example 93,

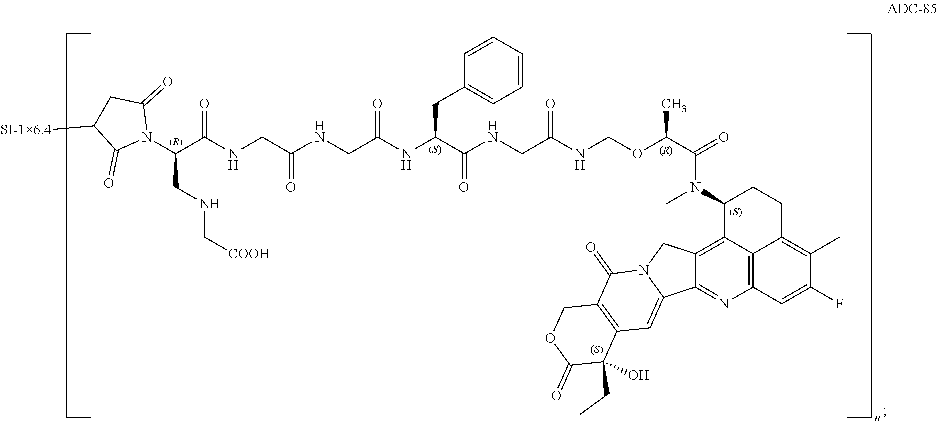

### Example 180

ADC-86 was prepared according to the general-purpose coupling method of Example 93,

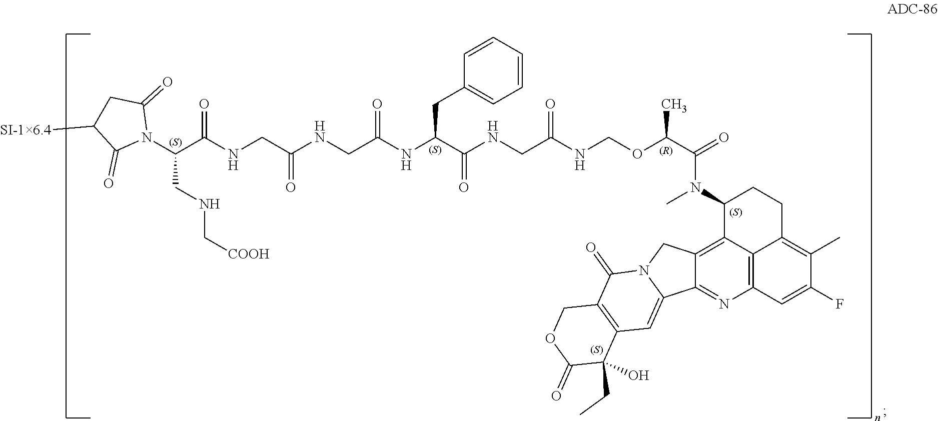

### Example 181

ADC-87 was prepared according to the general-purpose coupling method of Example 93,

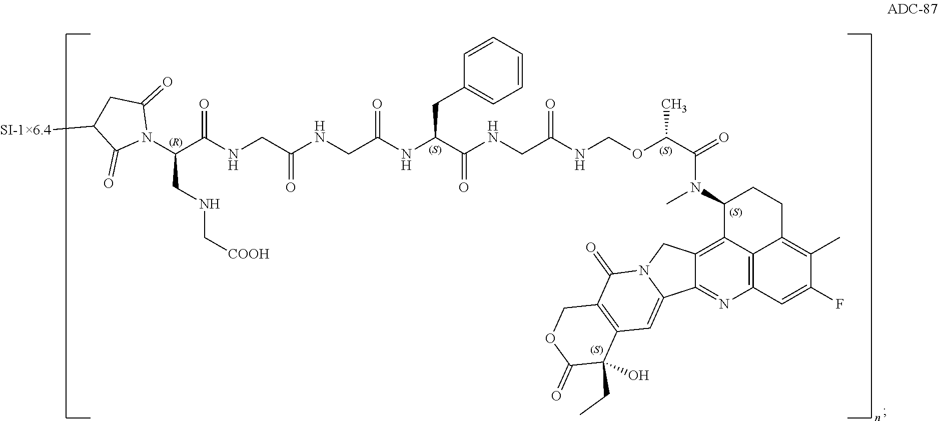

### Example 182

ADC-88 was prepared according to the general-purpose coupling method of Example 93,

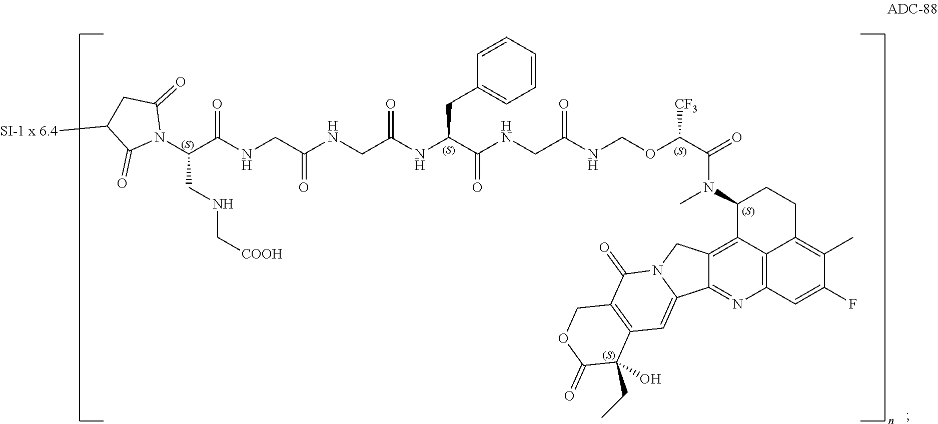

### Example 183

ADC-89 was prepared according to the general-purpose coupling method of Example 93,

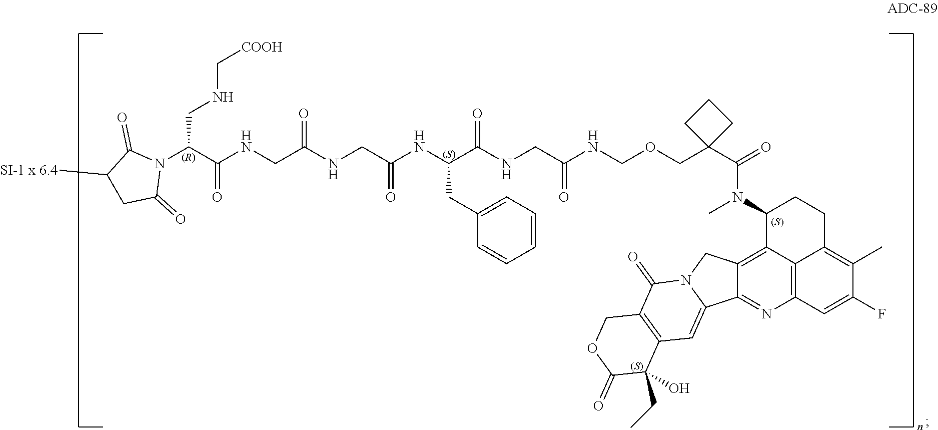

### Example 184

ADC-90 was prepared according to the general-purpose coupling method of Example 93,

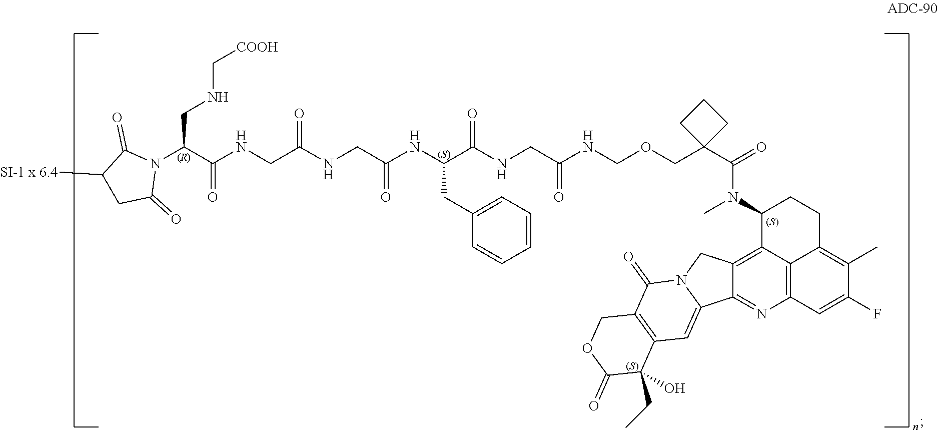

### Example 185

ADC-91 was prepared according to the general-purpose coupling method of Example 93,

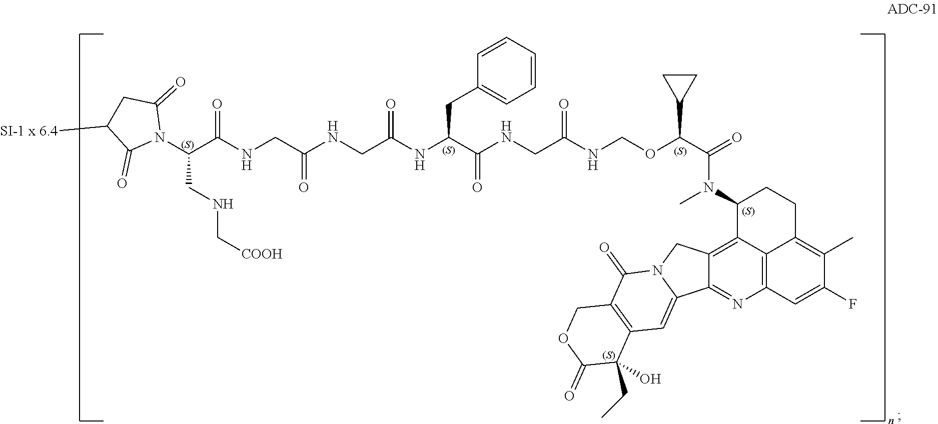

### Example 186

ADC-92 was prepared according to the general-purpose coupling method of Example 93,

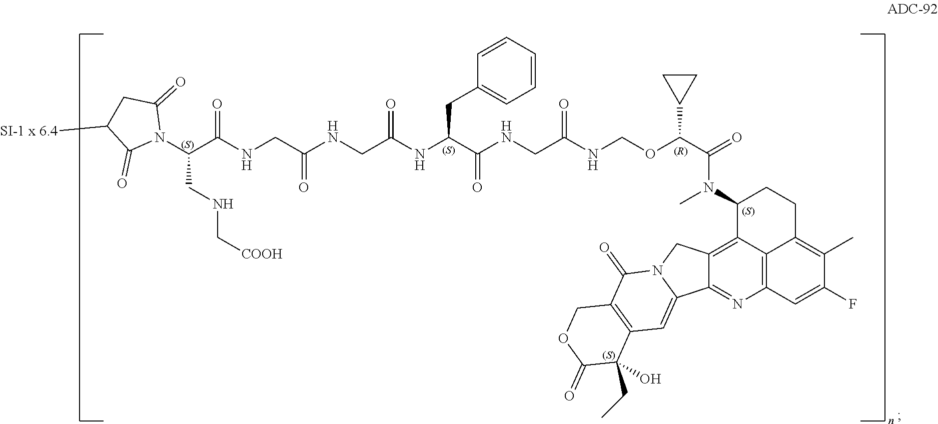

### Example 187

ADC-93 was prepared according to the general-purpose coupling method of Example 93,

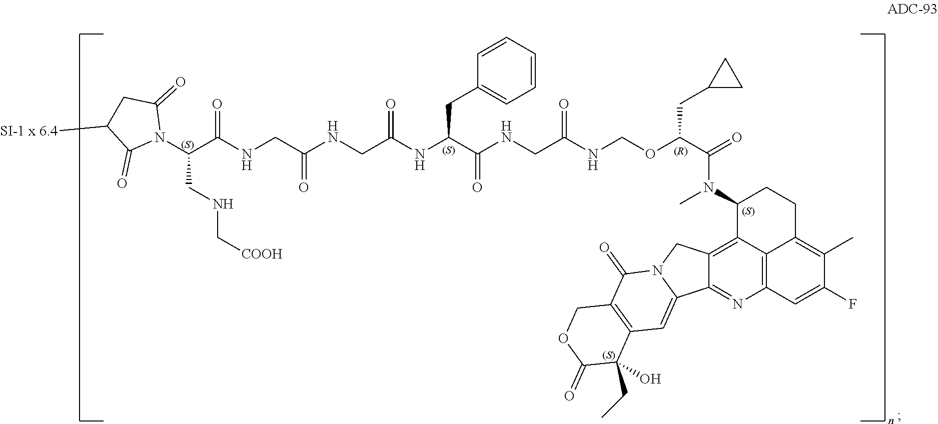

### Example 188

ADC-94 was prepared according to the general-purpose coupling method of Example 93,

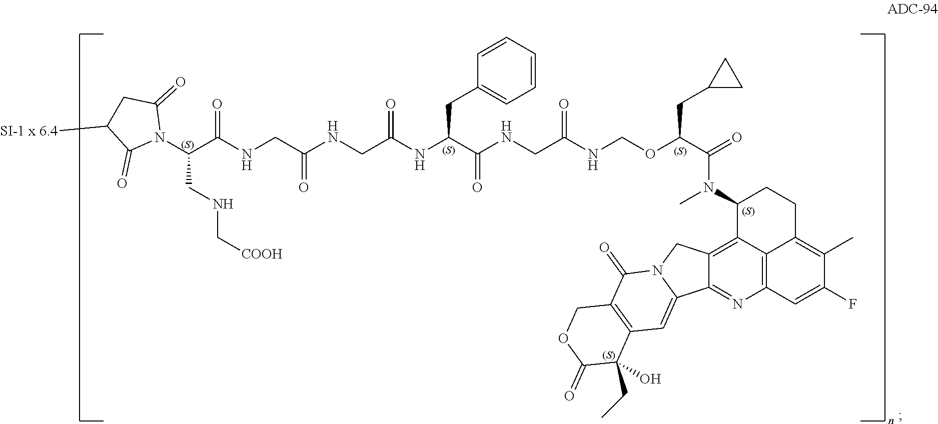

### Example 189

ADC-95 was prepared according to the general-purpose coupling method of Example 93,

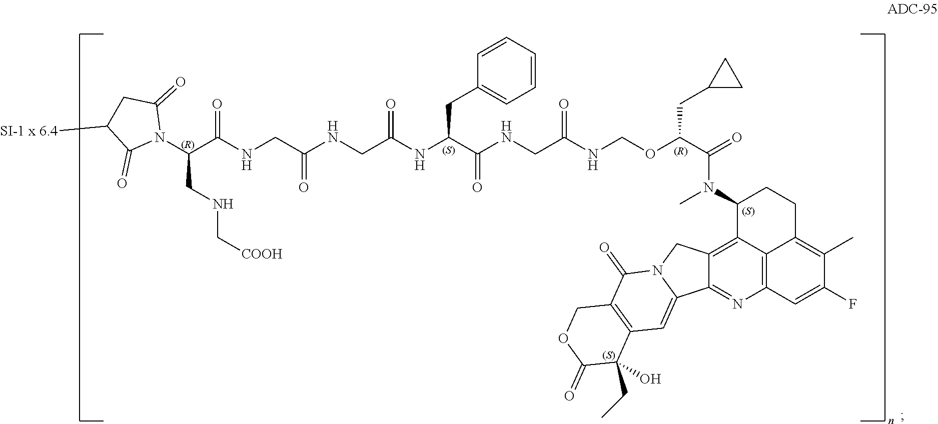

### Example 190

ADC-96 was prepared according to the general-purpose coupling method of Example 93,

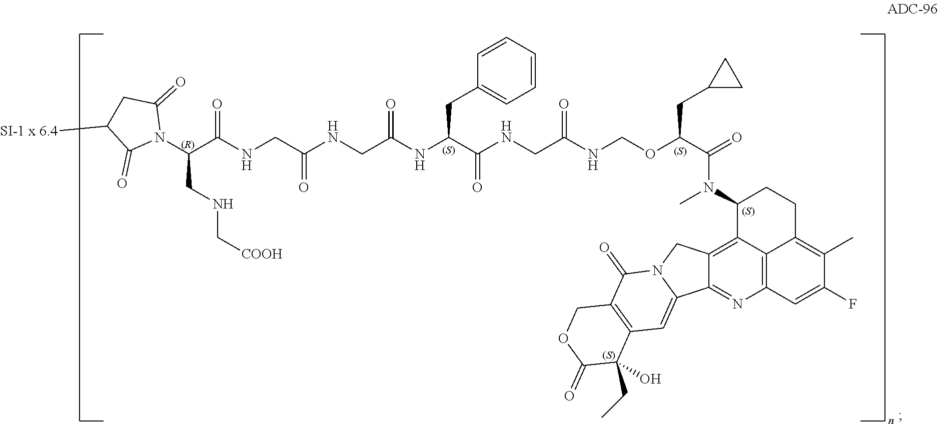

### Example 191

ADC-97 was prepared according to the general-purpose coupling method of Example 93,

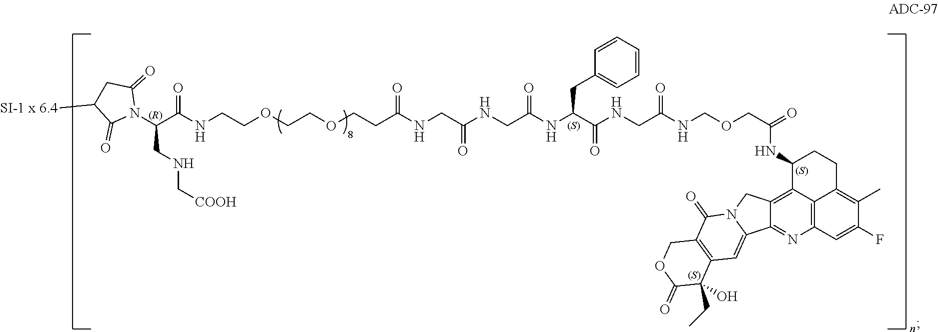

### Example 192

ADC-98 was prepared according to the general-purpose coupling method of Example 93,

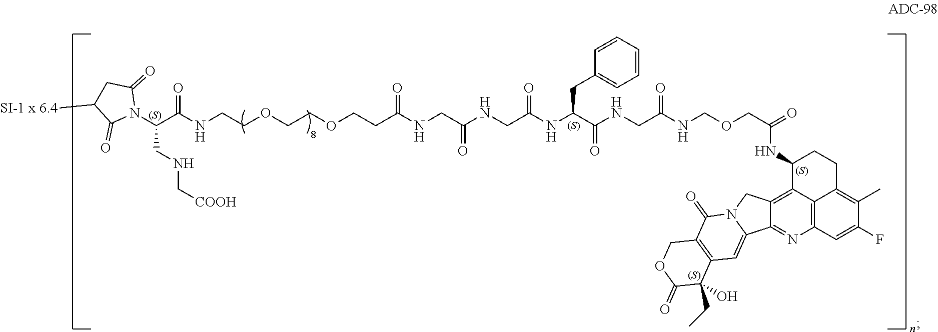

### Example 193

ADC-99 was prepared according to the general-purpose coupling method of Example 93,

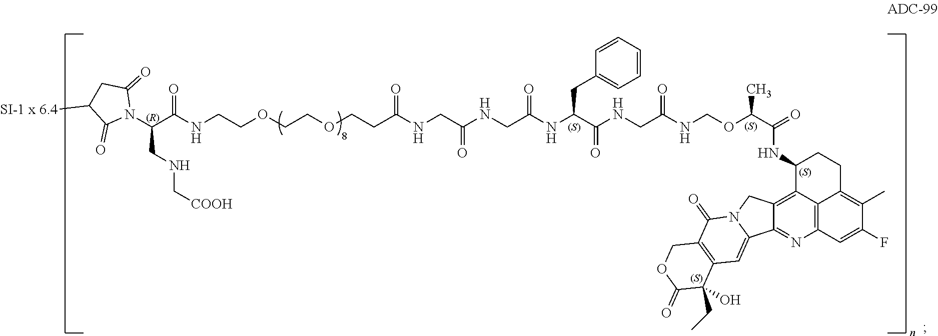

### Example 194

ADC-100 was prepared according to the general-purpose coupling method of Example 93,

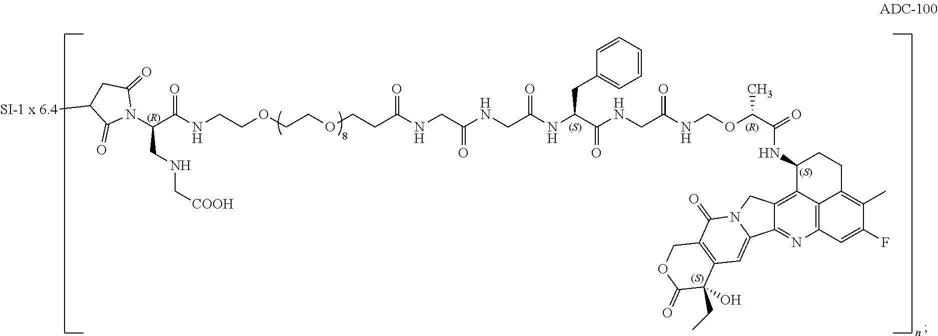

### Example 195

ADC-101 was prepared according to the general-purpose coupling method of Example 93,

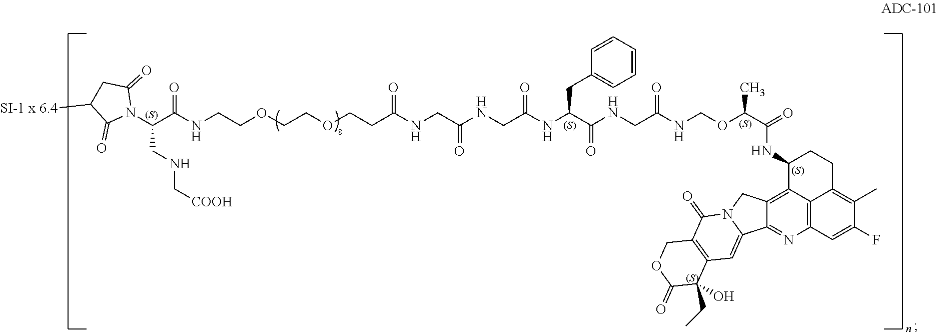

### Example 196

ADC-102 was prepared according to the general-purpose coupling method of Example 93,

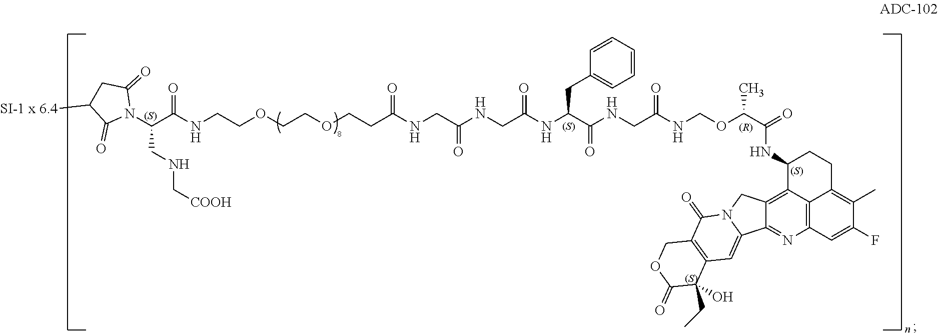

### Example 197

ADC-103 was prepared according to the general-purpose coupling method of Example 93,

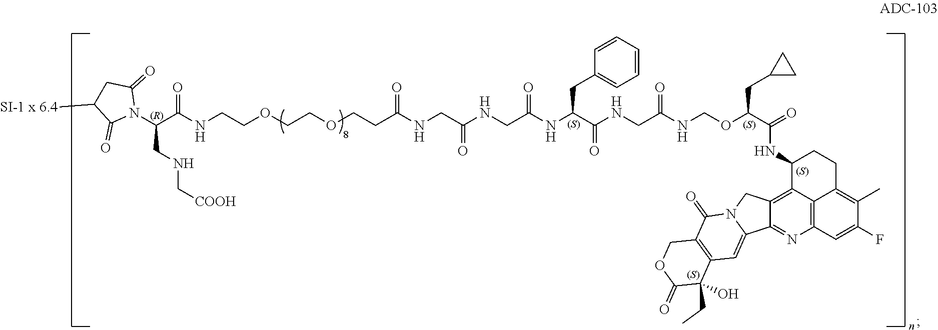

### Example 198

ADC-104 was prepared according to the general-purpose coupling method of Example 93,

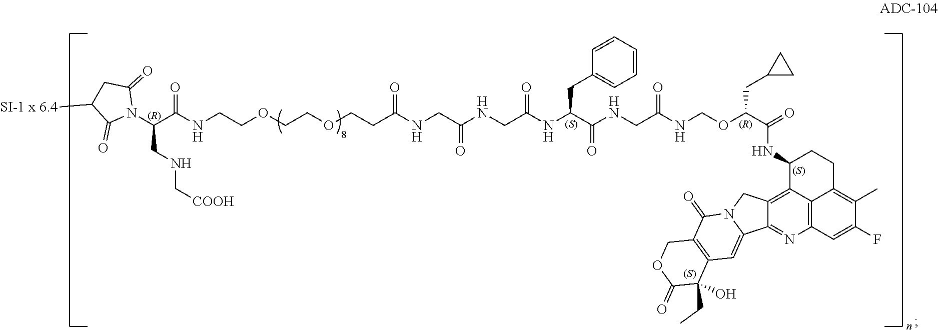

### Example 199

ADC-105 was prepared according to the general-purpose coupling method of Example 93,

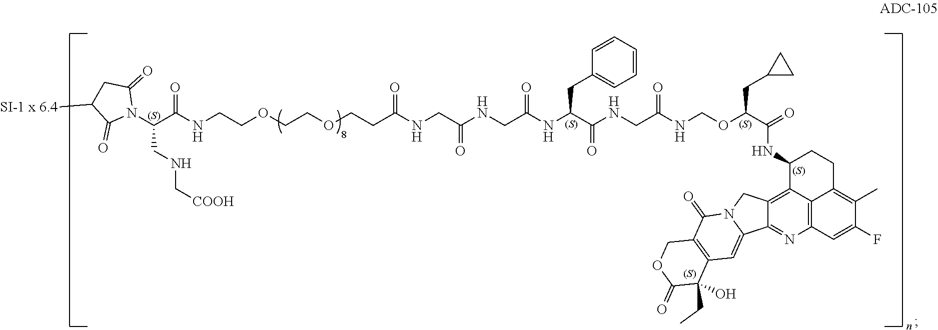

### Example 200

ADC-106 was prepared according to the general-purpose coupling method of Example 93,

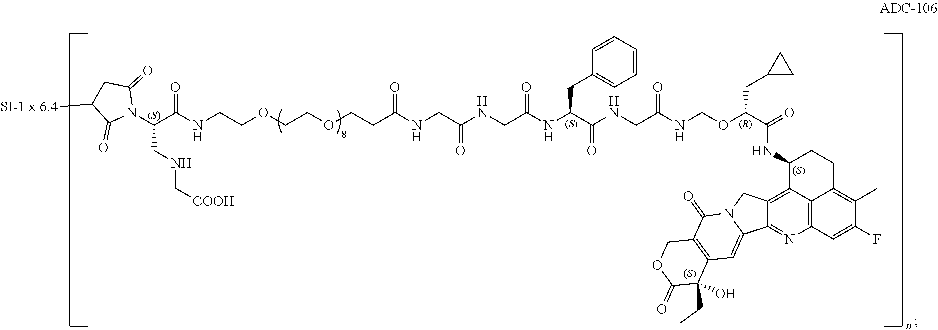

### Example 201

ADC-DS was prepared from compound 45 according to the general-purpose coupling method of Example 93,

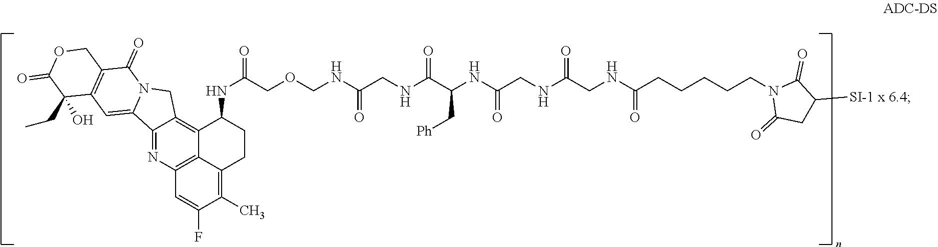

### Example 202

ADC-107 was prepared according to the general-purpose coupling method of Example 94,

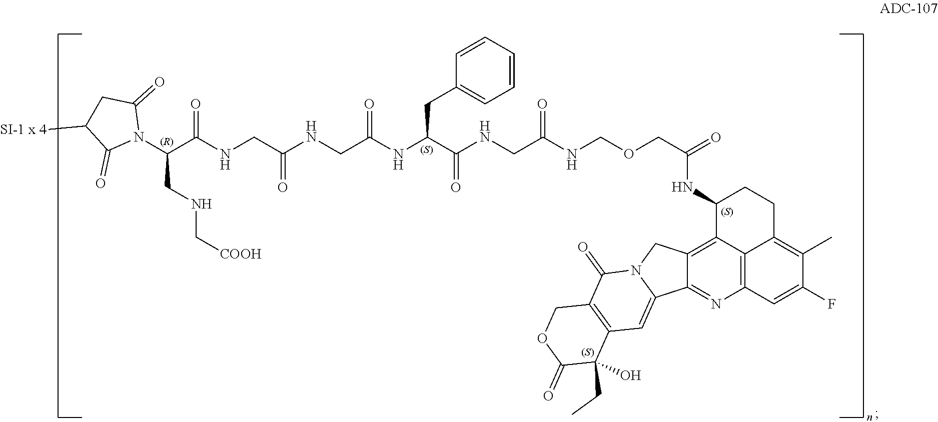

### Example 203

ADC-108 was prepared according to the general-purpose coupling method of Example 94,

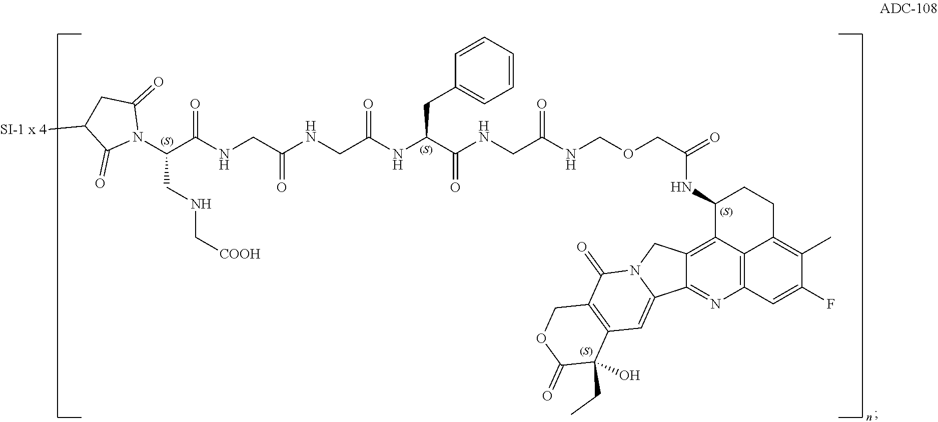

### Example 204

ADC-109 was prepared according to the general-purpose coupling method of Example 94,

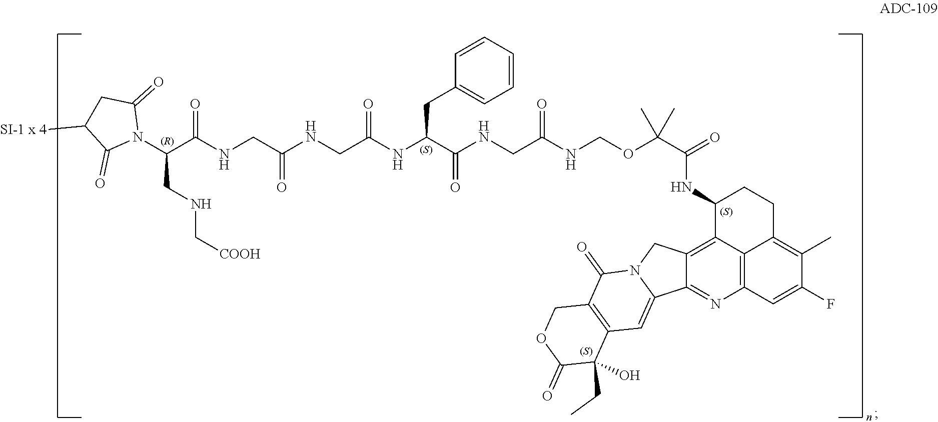

### Example 205

ADC-110 was prepared according to the general-purpose coupling method of Example 94,

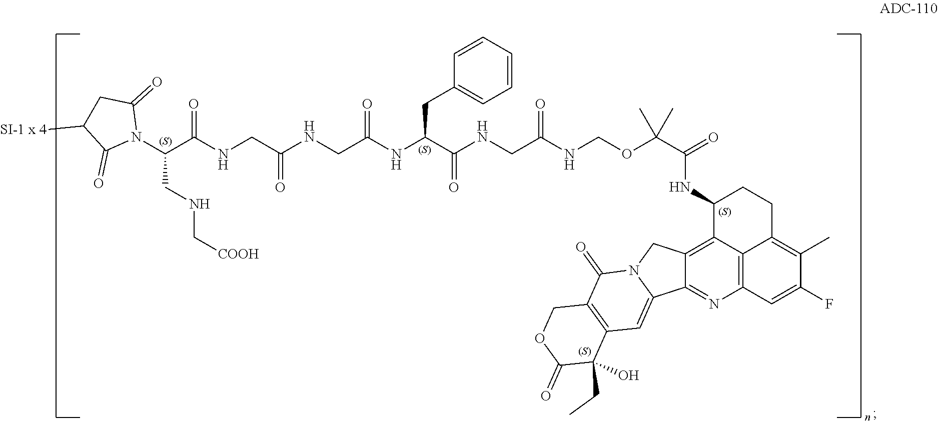

### Example 206

ADC-111 was prepared according to the general-purpose coupling method of Example 94,

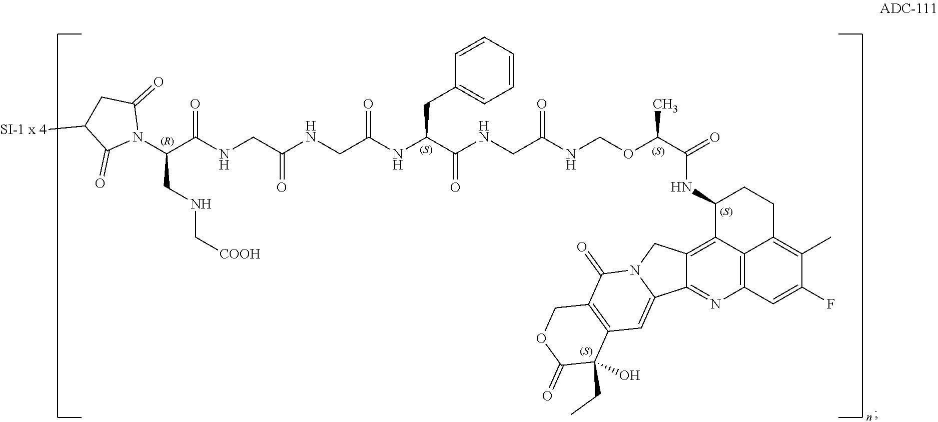

### Example 207

ADC-112 was prepared according to the general-purpose coupling method of Example 94,

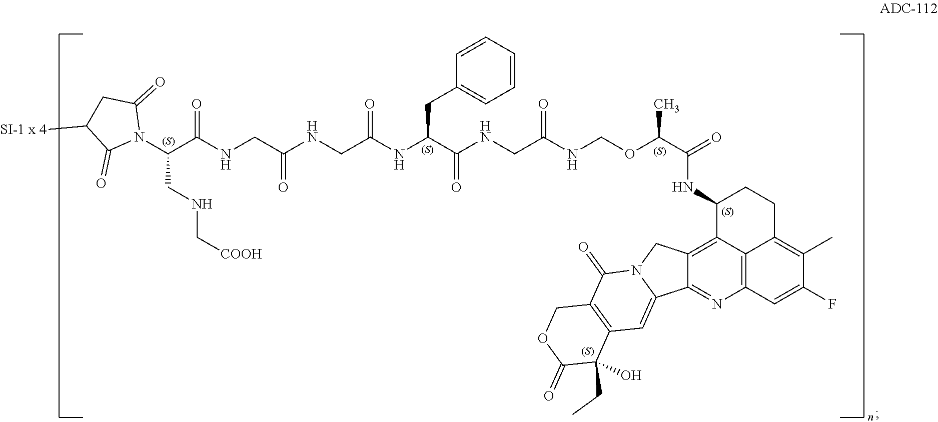

### Example 208

ADC-113 was prepared according to the general-purpose coupling method of Example 94,

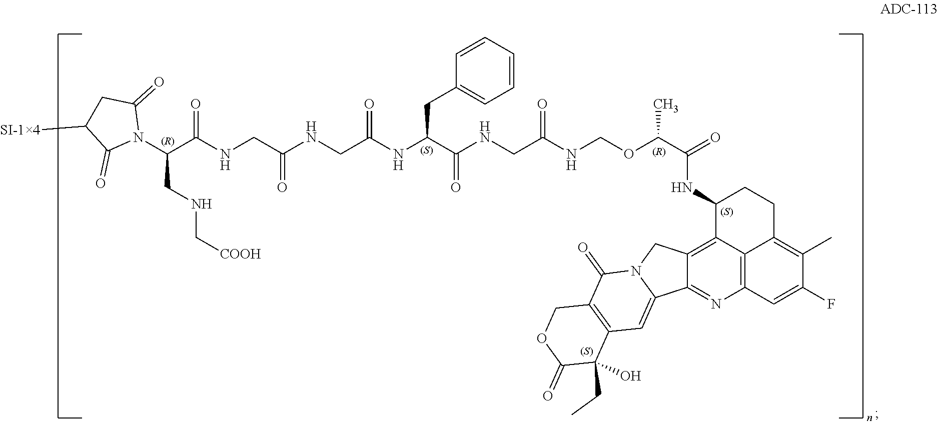

### Example 209

ADC-114 was prepared according to the general-purpose coupling method of Example 94,

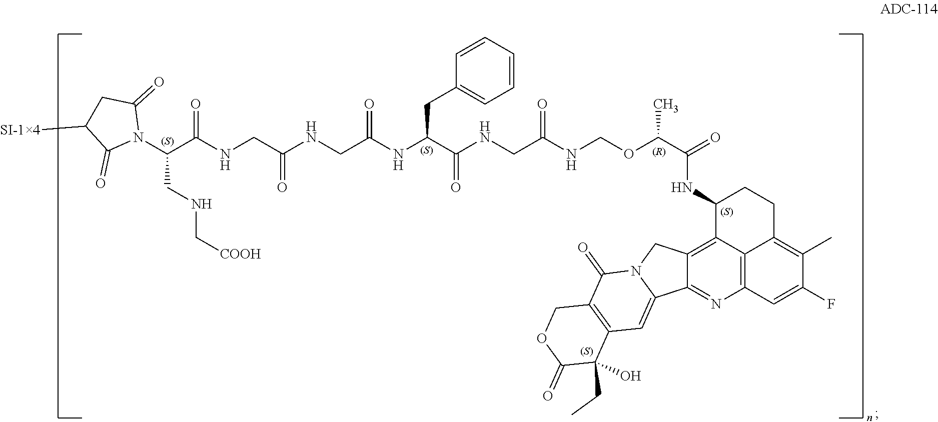

### Example 210

ADC-115 was prepared according to the general-purpose coupling method of Example 94,

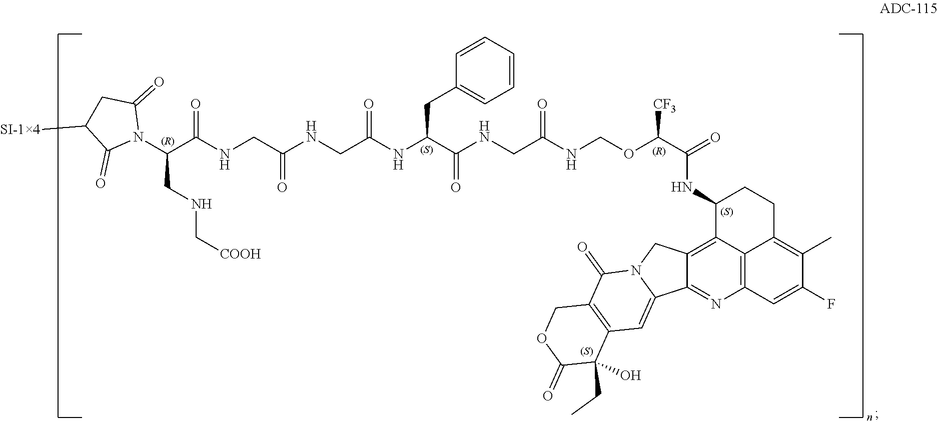

### Example 211

ADC-116 was prepared according to the general-purpose coupling method of Example 94,

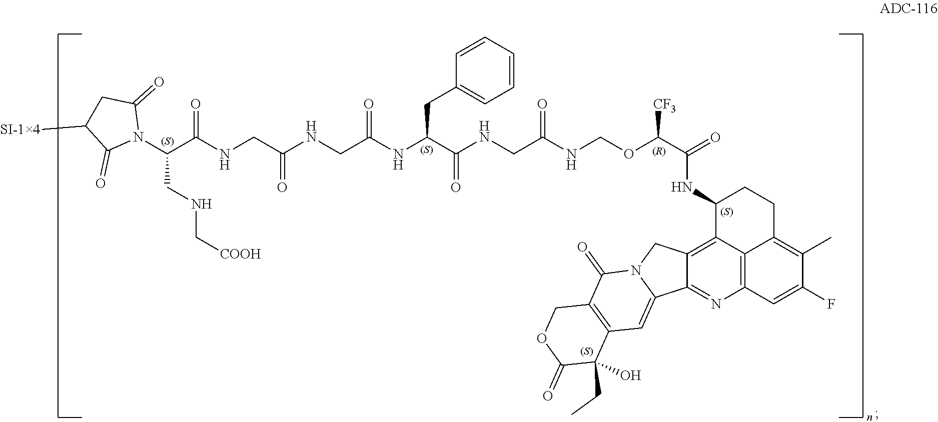

### Example 212

ADC-117 was prepared according to the general-purpose coupling method of Example 94,

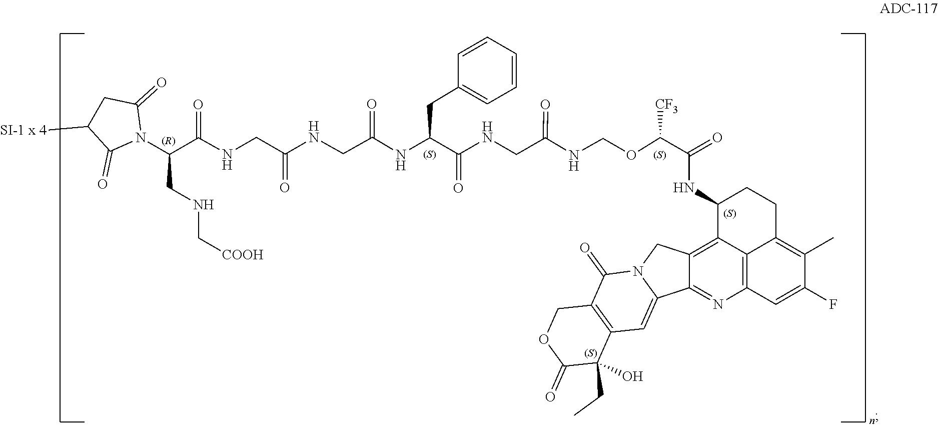

### Example 213

ADC-118 was prepared according to the general-purpose coupling method of Example 94,

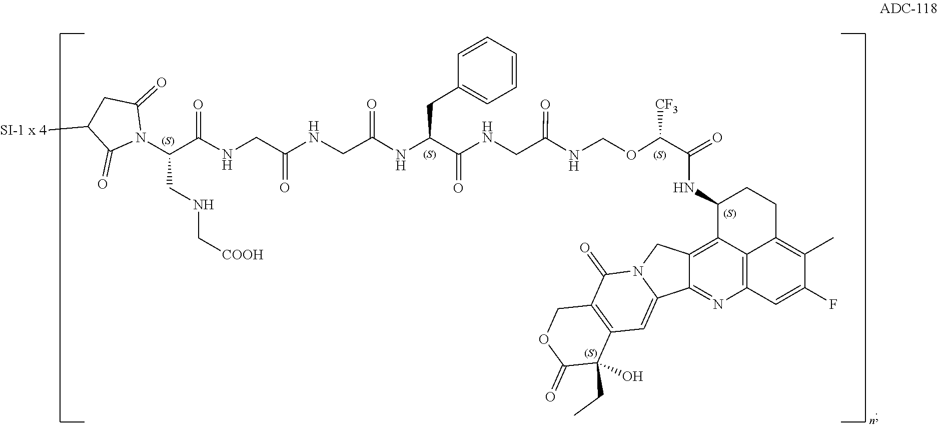

### Example 214

ADC-119 was prepared according to the general-purpose coupling method of Example 94,

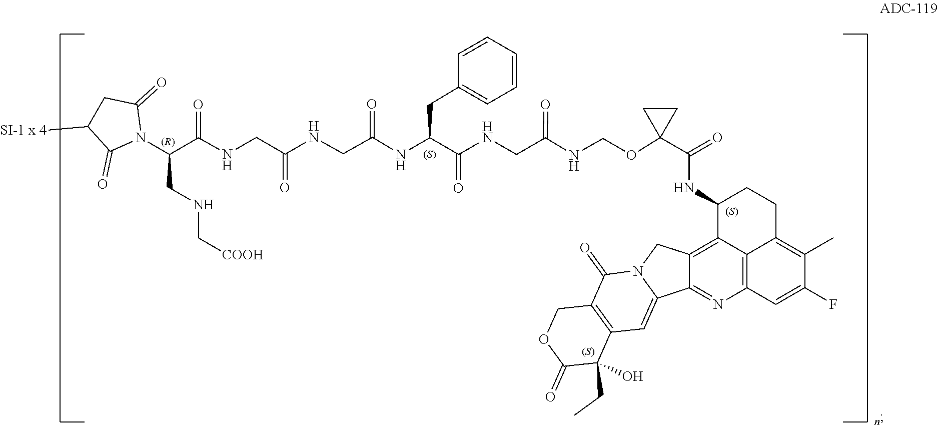

### Example 215

ADC-120 was prepared according to the general-purpose coupling method of Example 94,

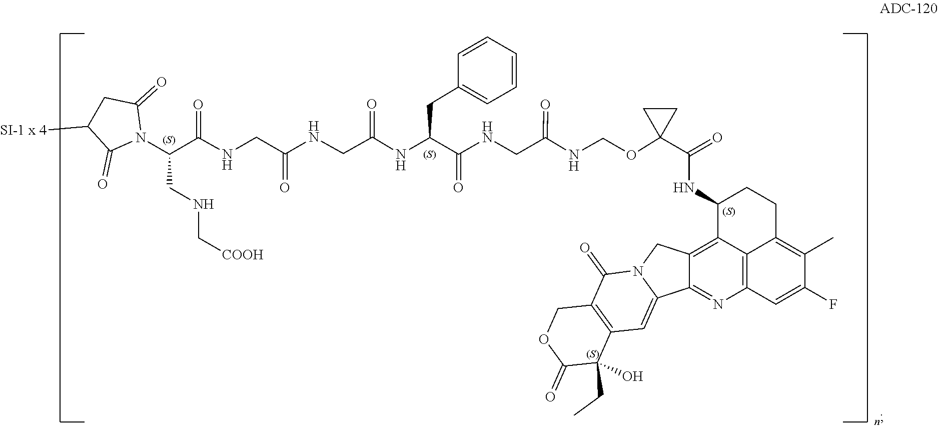

### Example 216

ADC-121 was prepared according to the general-purpose coupling method of Example 94,

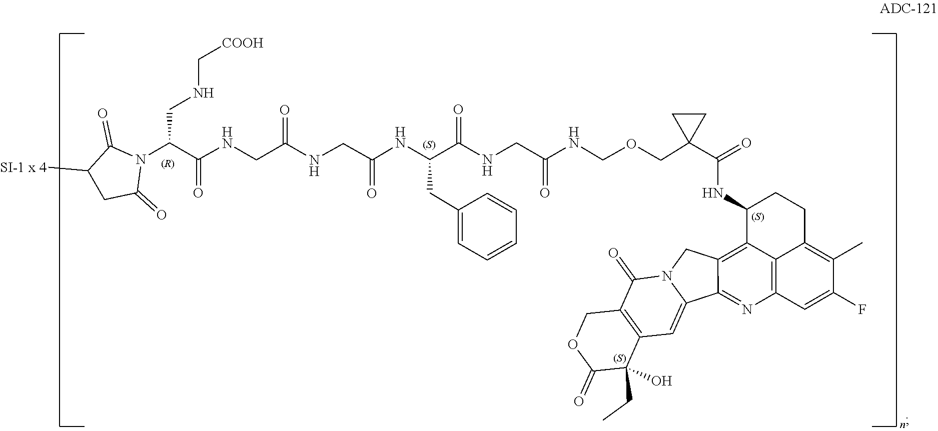

### Example 217

ADC-122 was prepared according to the general-purpose coupling method of Example 94,

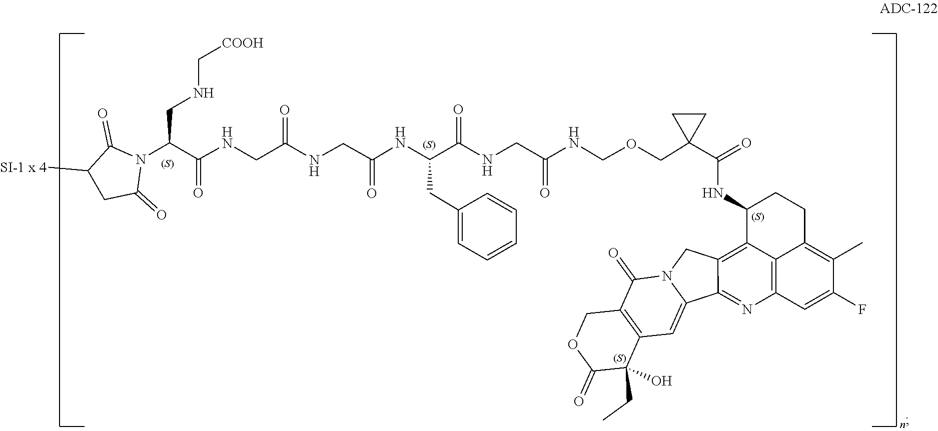

### Example 218

ADC-123 was prepared according to the general-purpose coupling method of Example 94,

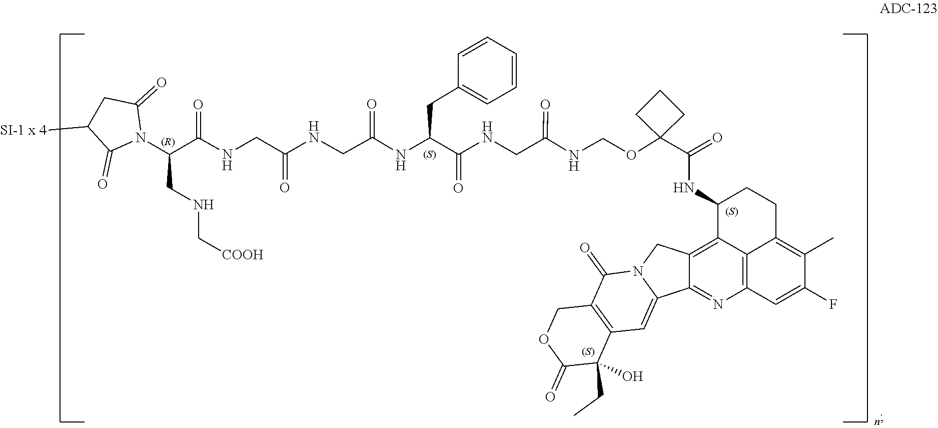

### Example 219

ADC-124 was prepared according to the general-purpose coupling method of Example 94,

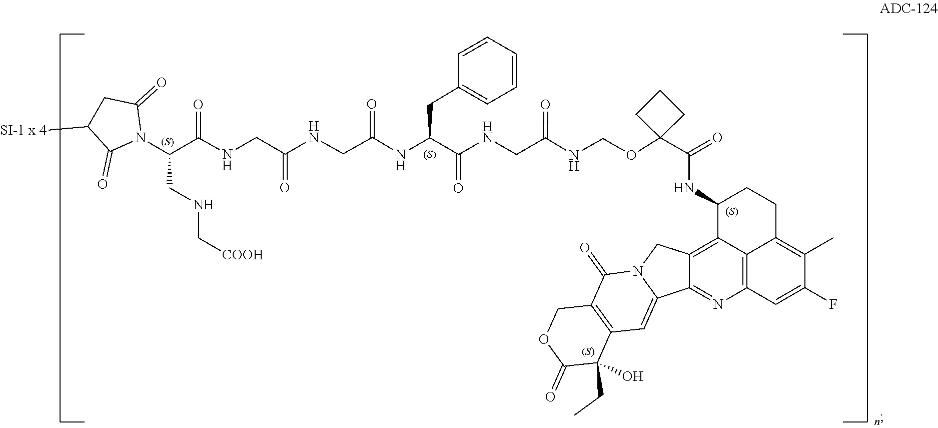

### Example 220

ADC-125 was prepared according to the general-purpose coupling method of Example 94,

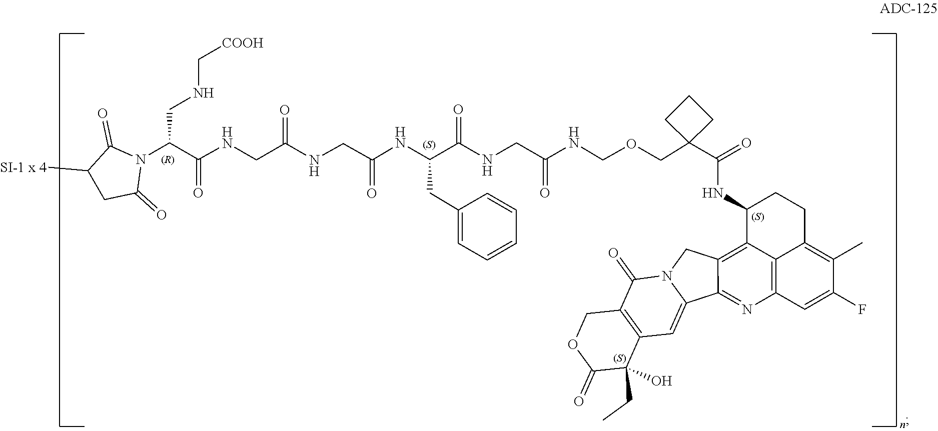

### Example 221

ADC-126 was prepared according to the general-purpose coupling method of Example 94,

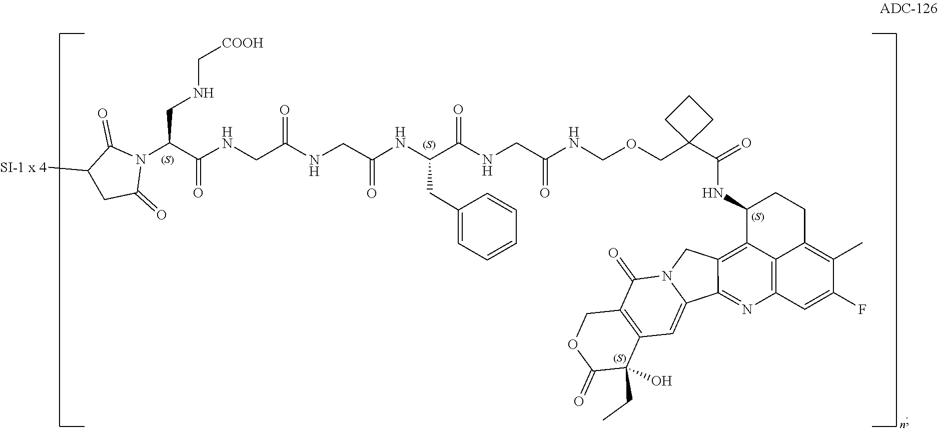

### Example 222

ADC-127 was prepared according to the general-purpose coupling method of Example 94,

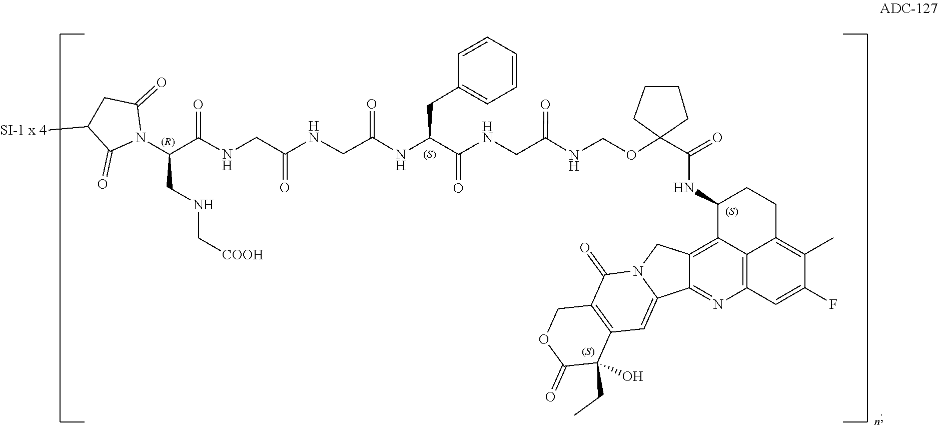

### Example 223

ADC-128 was prepared according to the general-purpose coupling method of Example 94,

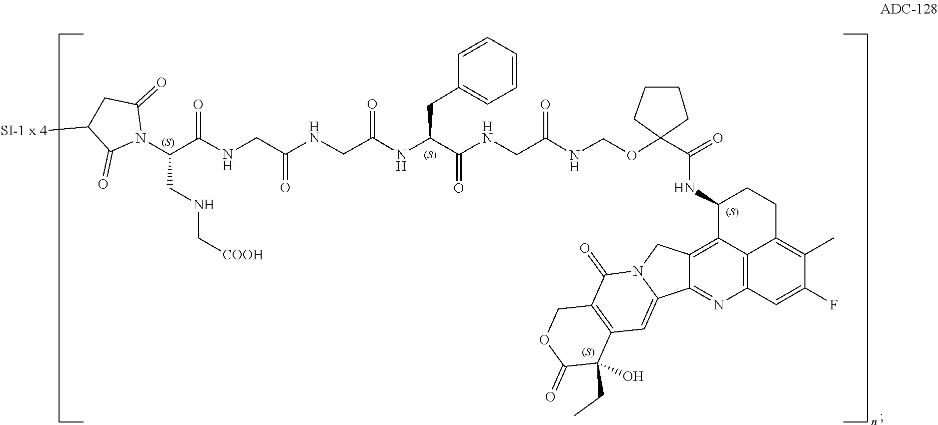

### Example 224

ADC-129 was prepared according to the general-purpose coupling method of Example 94,

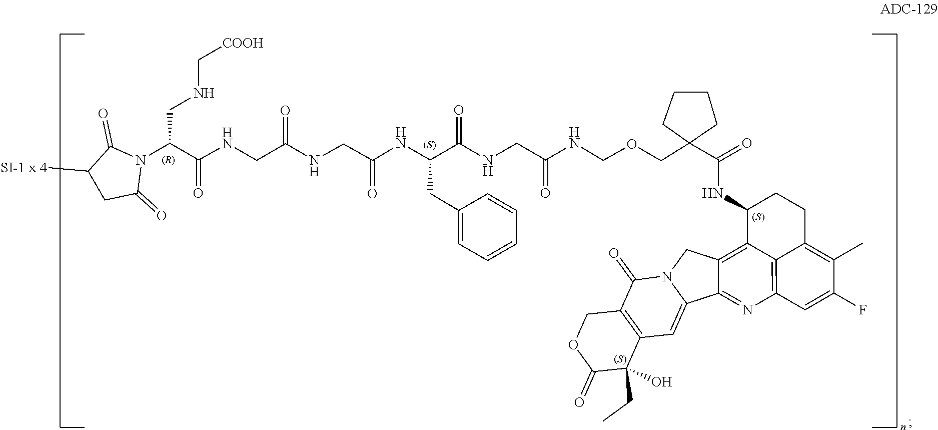

### Example 225

ADC-130 was prepared according to the general-purpose coupling method of Example 94,

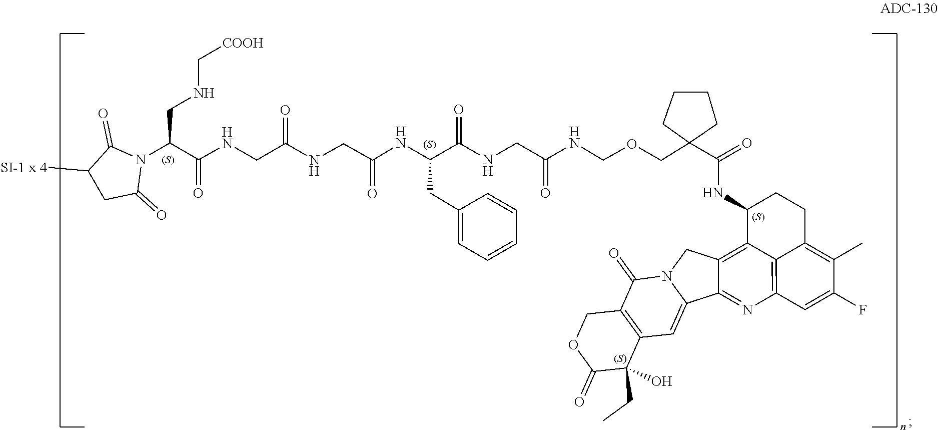

### Example 226

ADC-131 was prepared according to the general-purpose coupling method of Example 94,

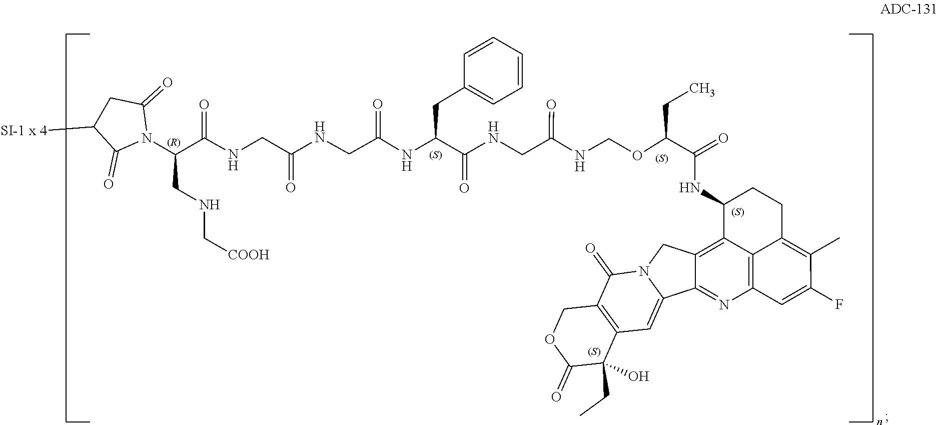

### Example 227

ADC-132 was prepared according to the general-purpose coupling method of Example 94,

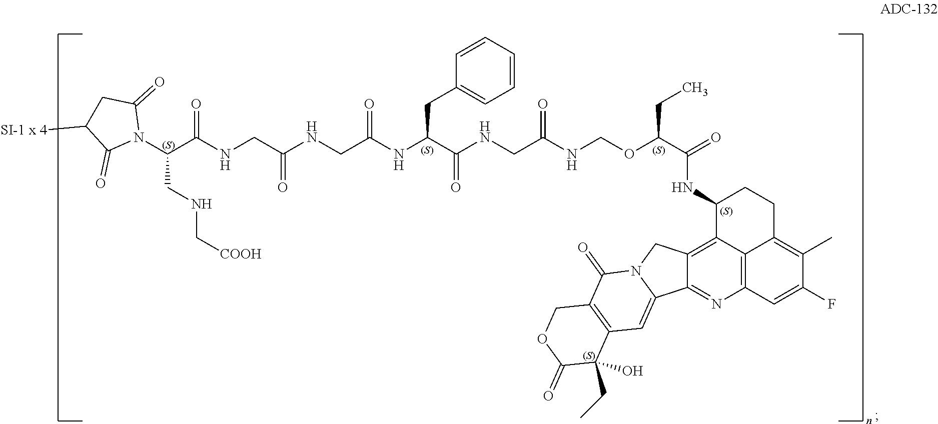

### Example 228

ADC-133 was prepared according to the general-purpose coupling method of Example 94,

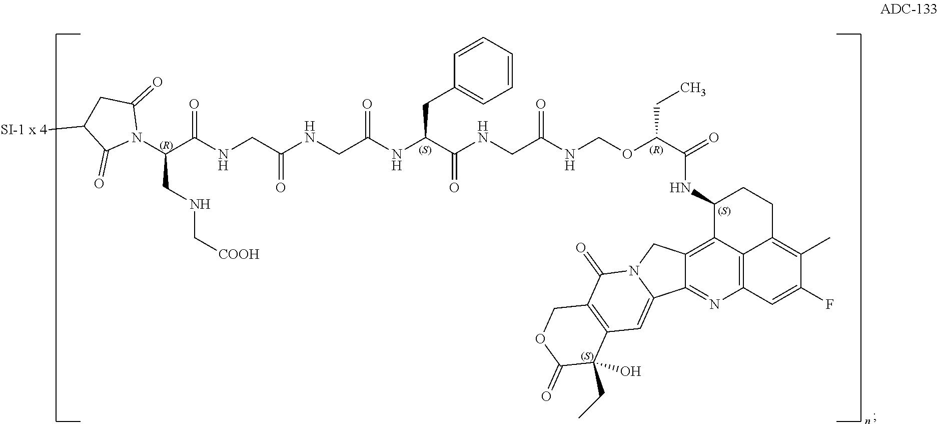

### Example 229

ADC-134 was prepared according to the general-purpose coupling method of Example 94,

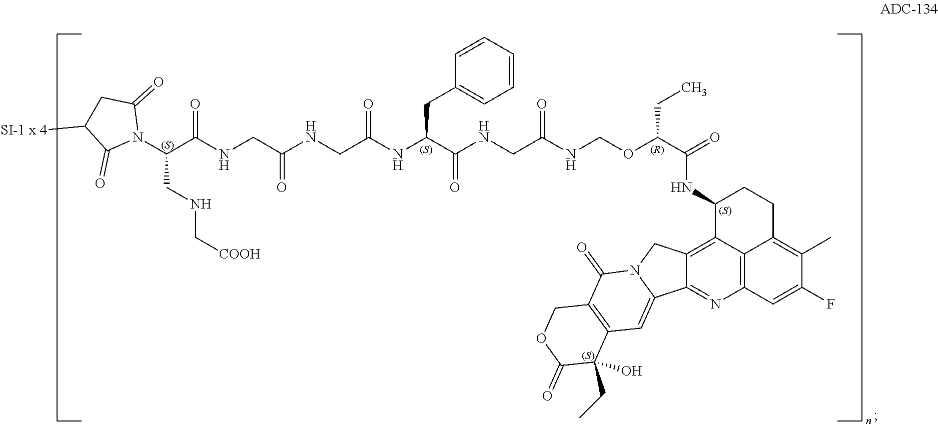

### Example 230

ADC-135 was prepared according to the general-purpose coupling method of Example 94,

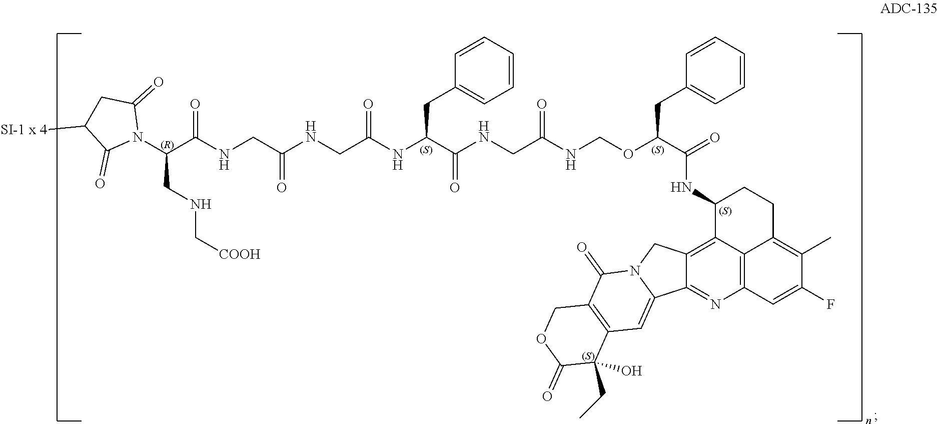

### Example 231

ADC-136 was prepared according to the general-purpose coupling method of Example 94,

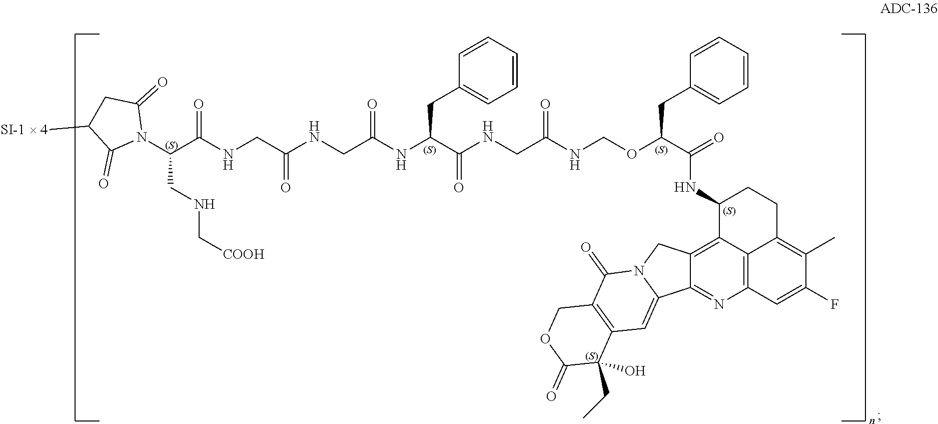

### Example 232

ADC-137 was prepared according to the general-purpose coupling method of Example 94,

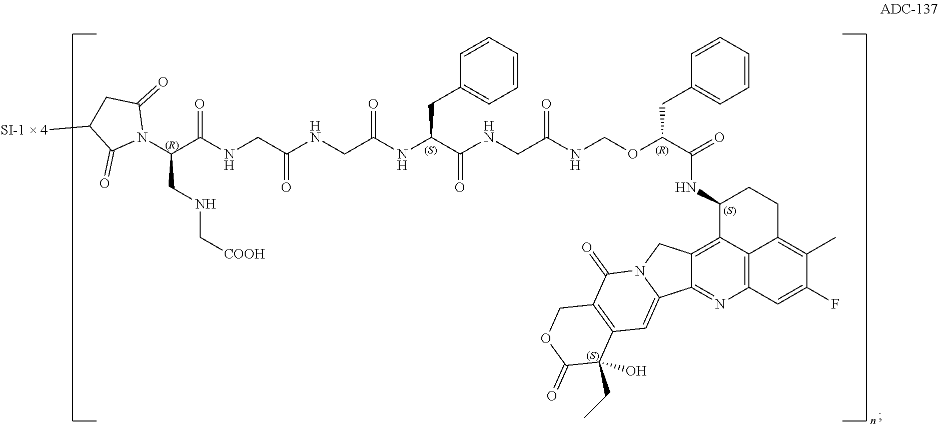

### Example 233

ADC-138 was prepared according to the general-purpose coupling method of Example 94,

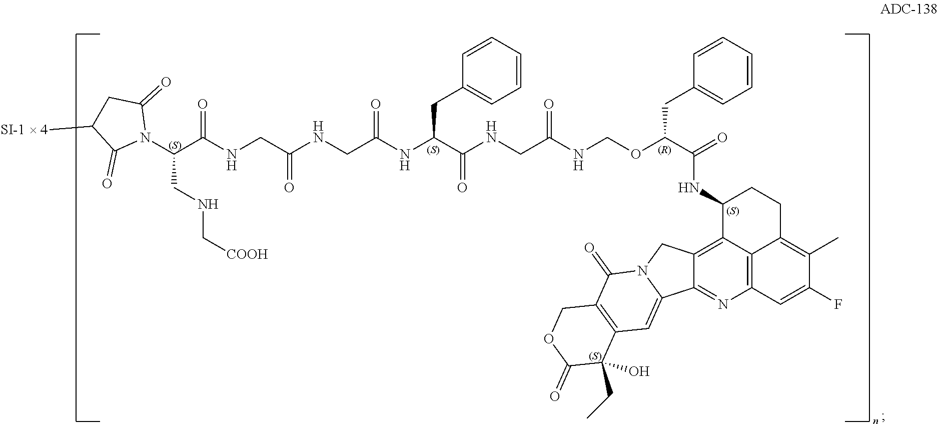

### Example 234

ADC-139 was prepared according to the general-purpose coupling method of Example 94,

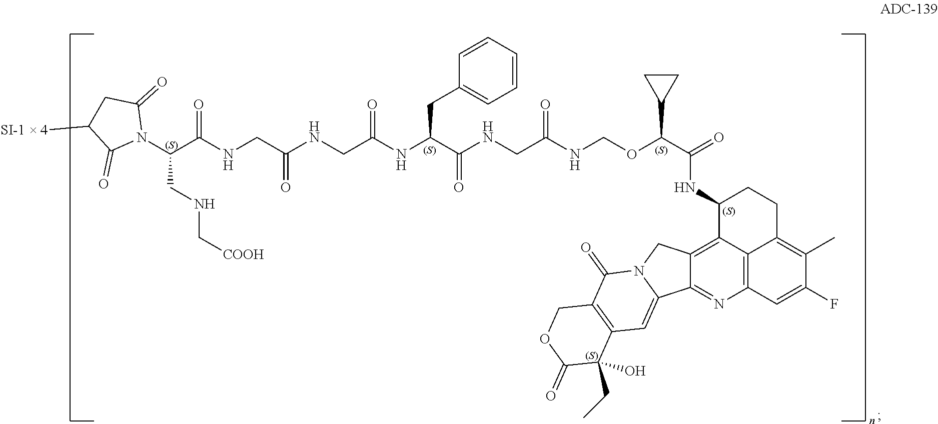

### Example 235

ADC-140 was prepared according to the general-purpose coupling method of Example 94,

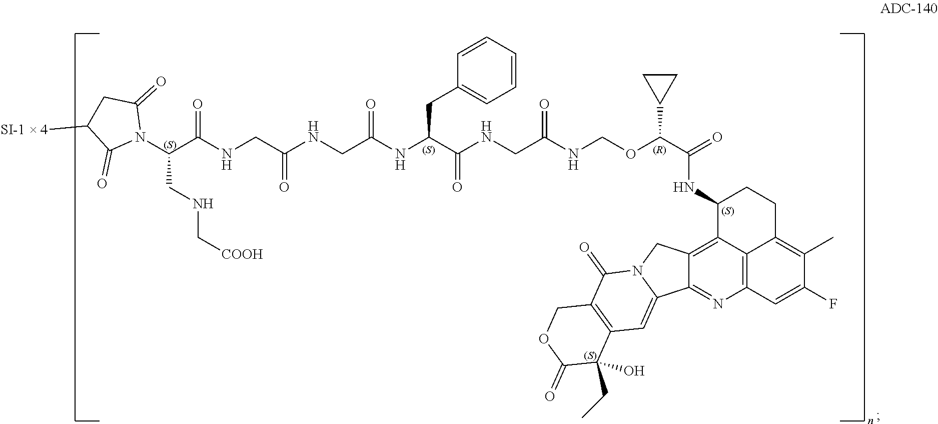

### Example 236

ADC-141 was prepared according to the general-purpose coupling method of Example 94,

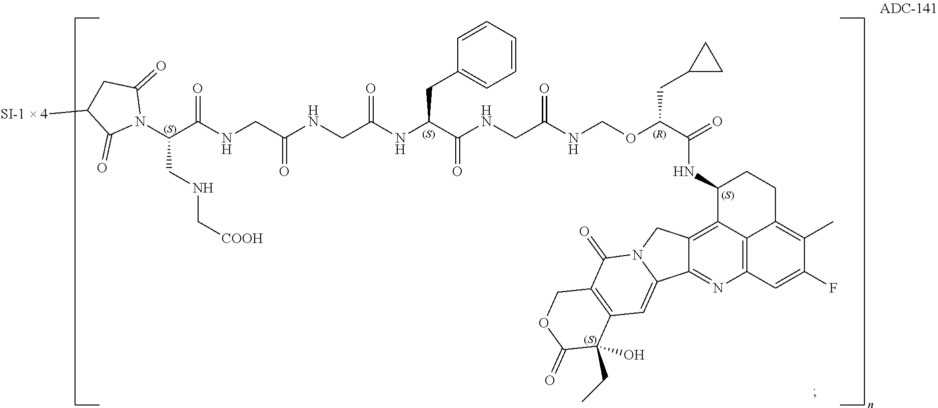

### Example 237

ADC-142 was prepared according to the general-purpose coupling method of Example 94,

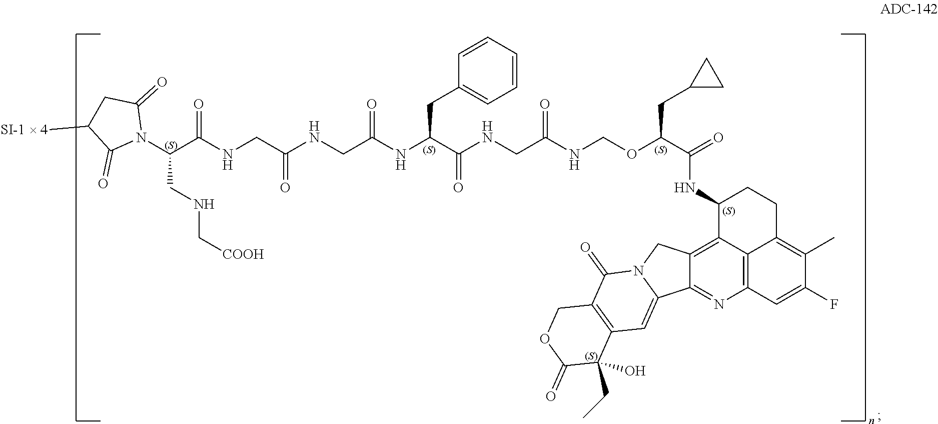

### Example 238

ADC-143 was prepared according to the general-purpose coupling method of Example 94,

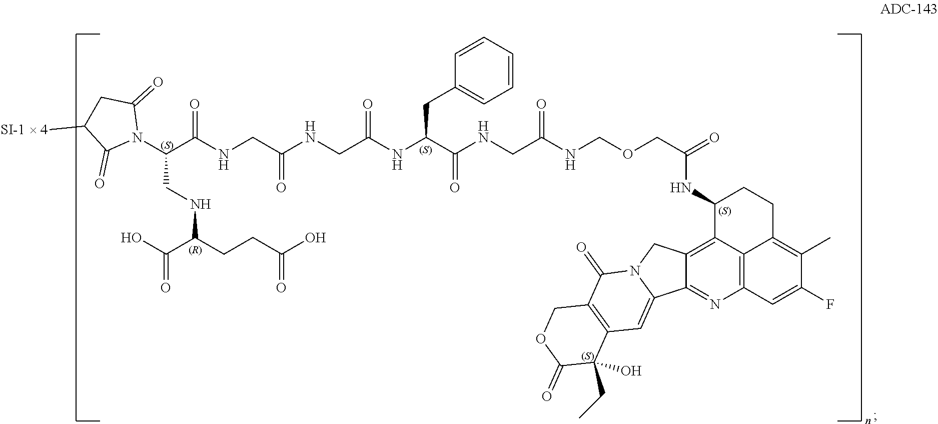

### Example 239

ADC-144 was prepared according to the general-purpose coupling method of Example 94,

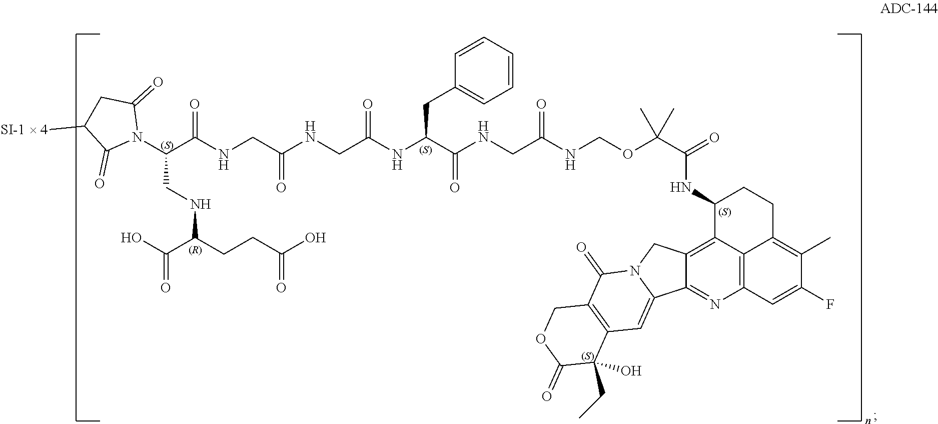

### Example 240

ADC-145 was prepared according to the general-purpose coupling method of Example 94,

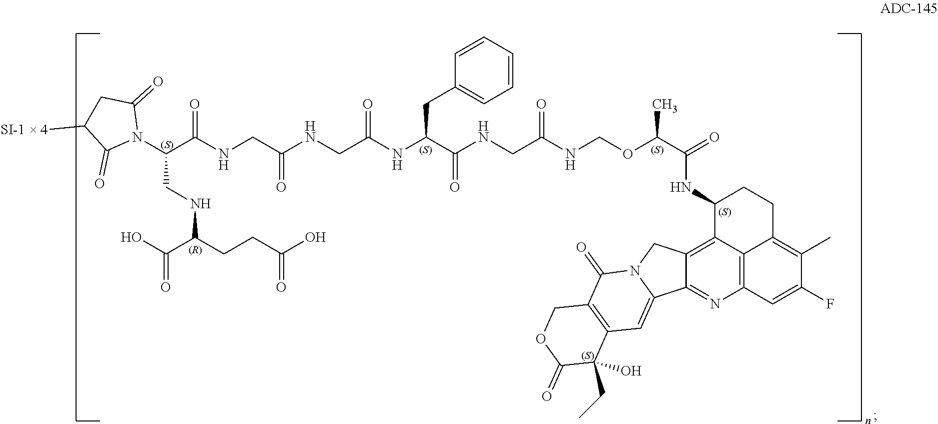

### Example 241

ADC-146 was prepared according to the general-purpose coupling method of Example 94,

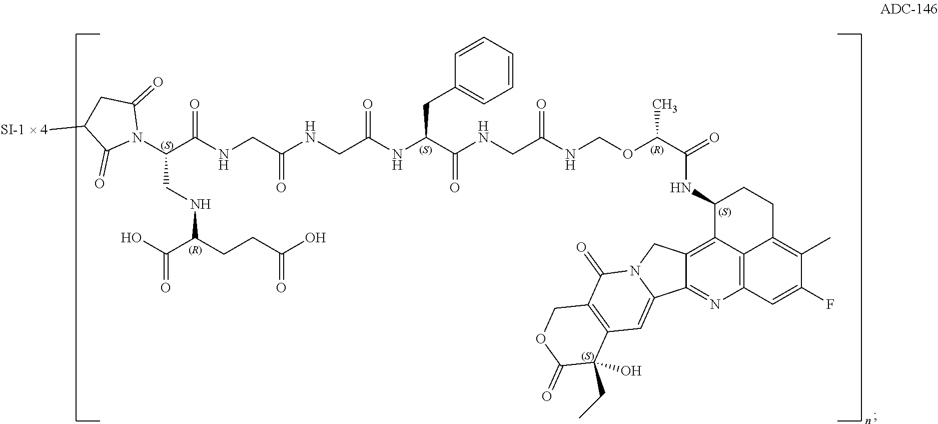

### Example 242

ADC-147 was prepared according to the general-purpose coupling method of Example 94,

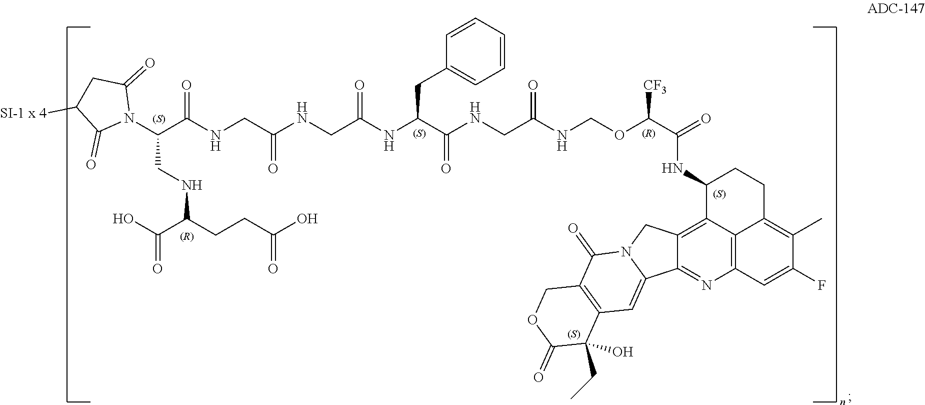

### Example 243

ADC-148 was prepared according to the general-purpose coupling method of Example 94,

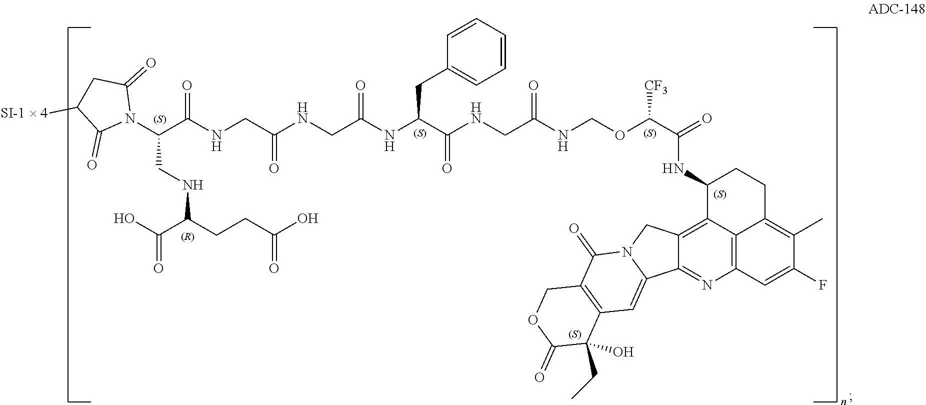

### Example 244

ADC-149 was prepared according to the general-purpose coupling method of Example 94,

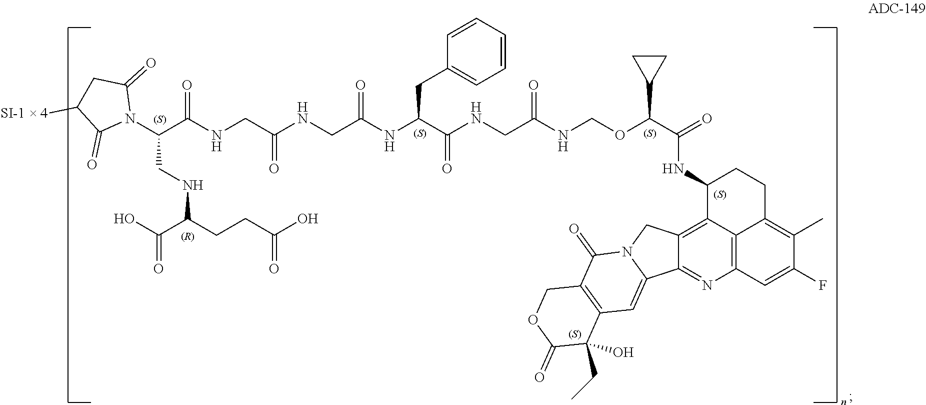

### Example 245

ADC-150 was prepared according to the general-purpose coupling method of Example 94,

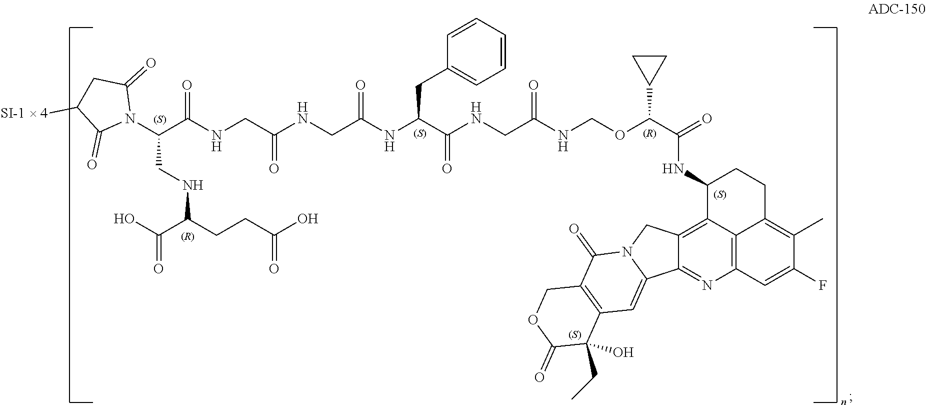

### Example 246

ADC-151 was prepared according to the general-purpose coupling method of Example 94,

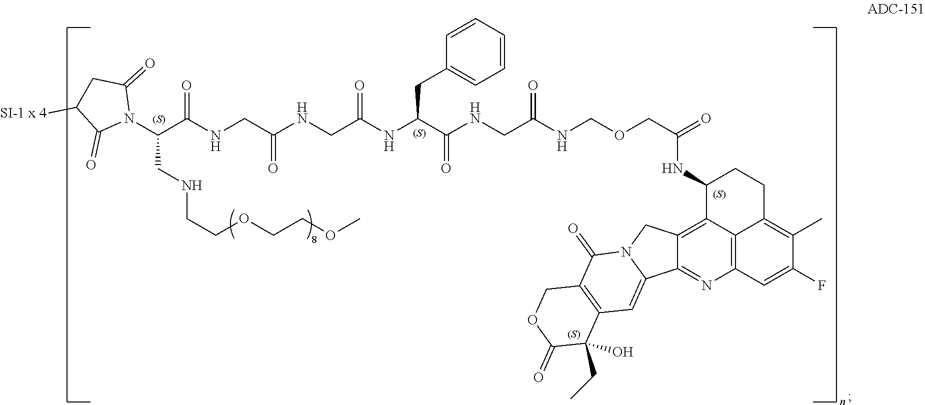

### Example 247

ADC-152 was prepared according to the general-purpose coupling method of Example 94,

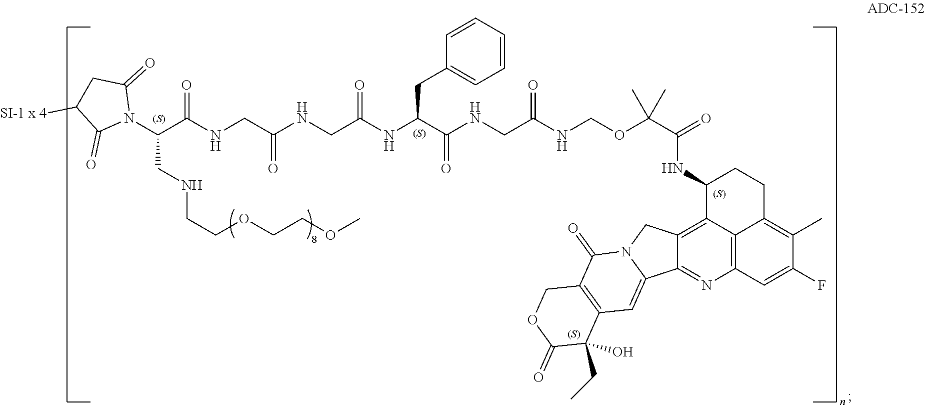

### Example 248

ADC-153 was prepared according to the general-purpose coupling method of Example 94,

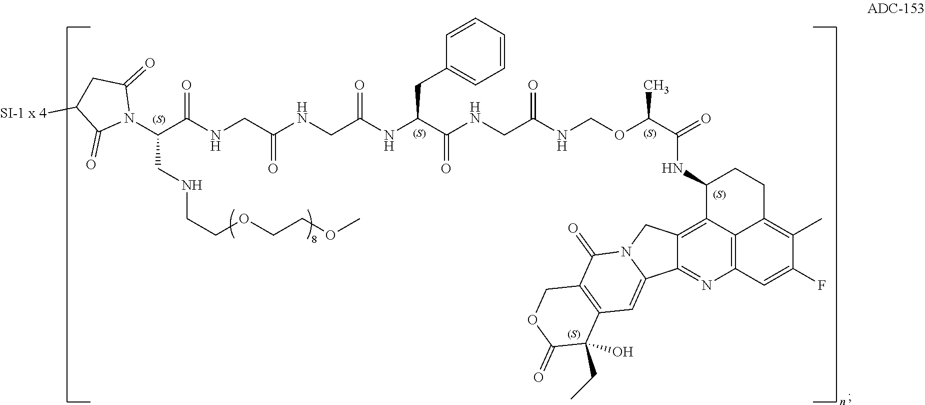

### Example 249

ADC-154 was prepared according to the general-purpose coupling method of Example 94,

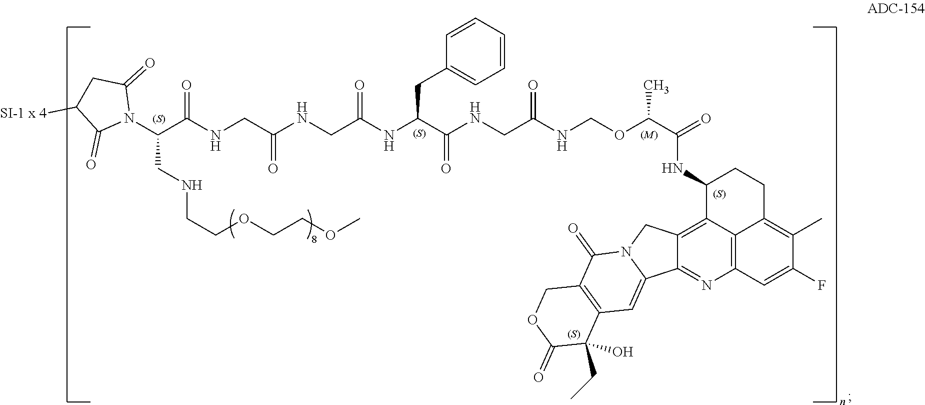

### Example 250

ADC-155 was prepared according to the general-purpose coupling method of Example 94,

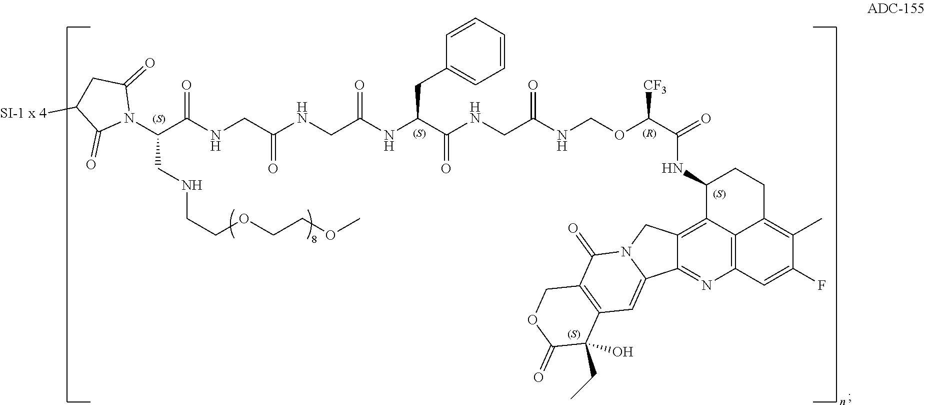

### Example 251

ADC-156 was prepared according to the general-purpose coupling method of Example 94,

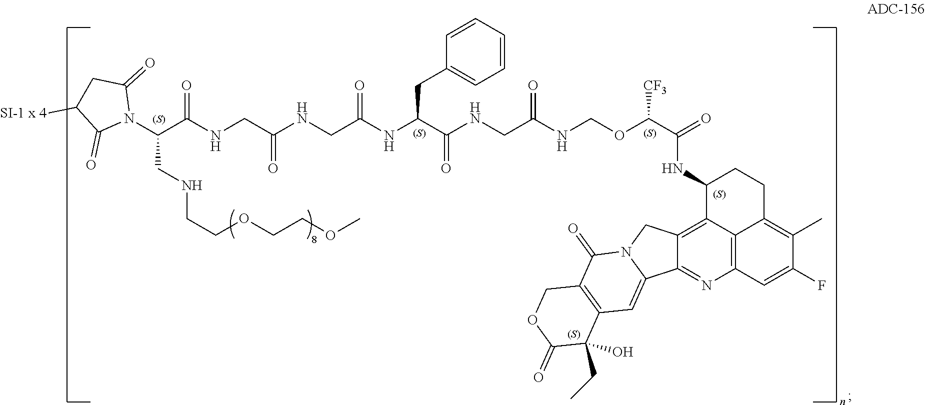

### Example 252

ADC-157 was prepared according to the general-purpose coupling method of Example 94,

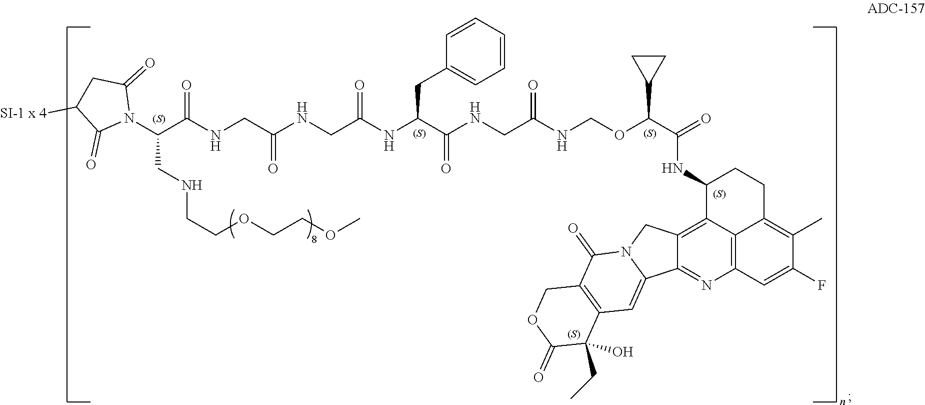

### Example 253

ADC-158 was prepared according to the general-purpose coupling method of Example 94,

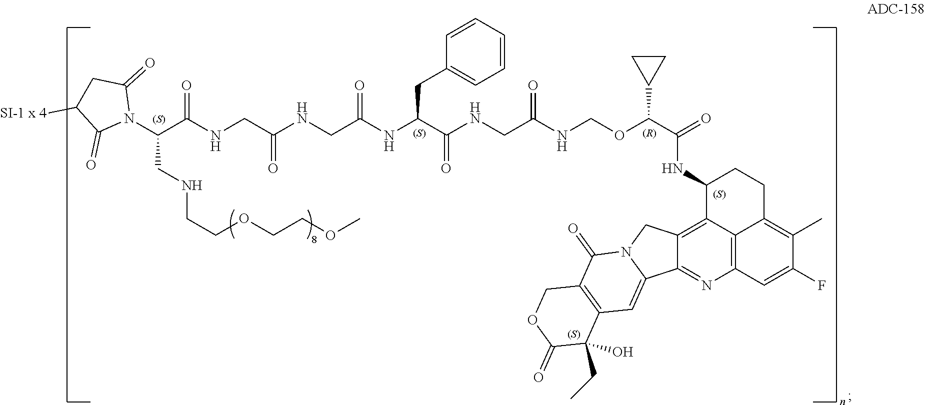

### Example 254

ADC-159 was prepared according to the general-purpose coupling method of Example 94,

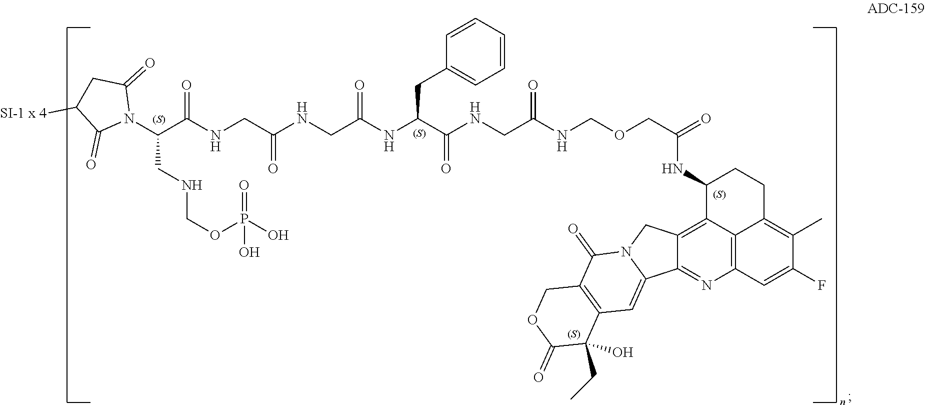

### Example 255

ADC-160 was prepared according to the general-purpose coupling method of Example 94,

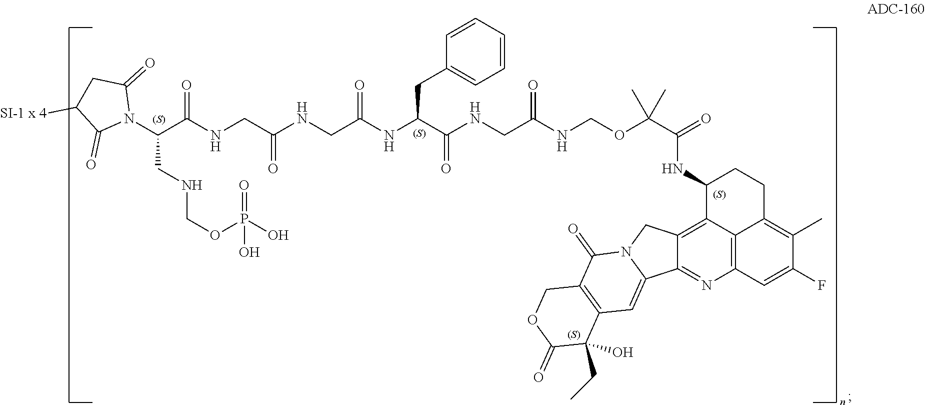

### Example 256

ADC-161 was prepared according to the general-purpose coupling method of Example 94,

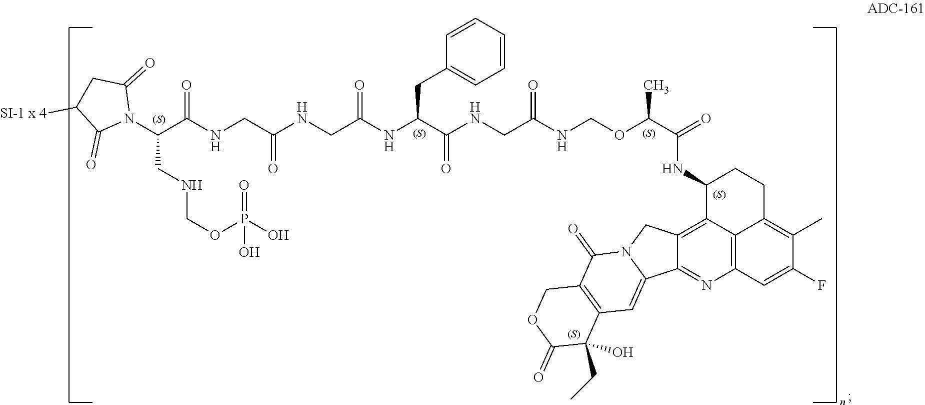

### Example 257

ADC-162 was prepared according to the general-purpose coupling method of Example 94,

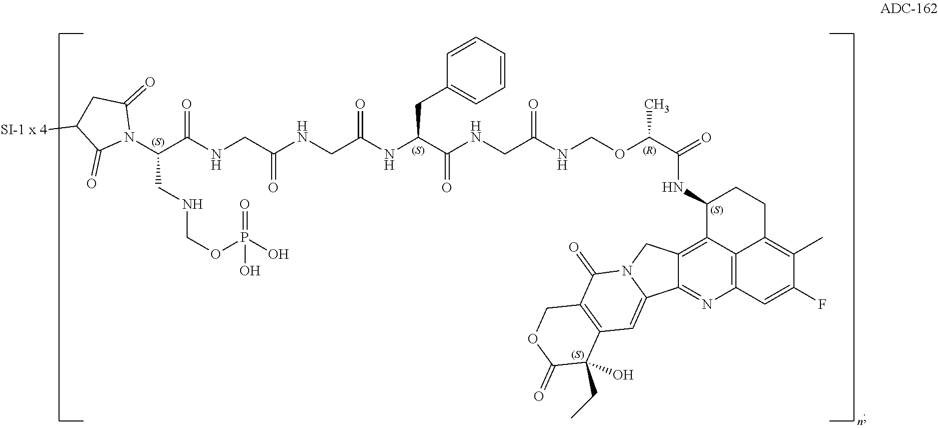

### Example 258

ADC-163 was prepared according to the general-purpose coupling method of Example 94,

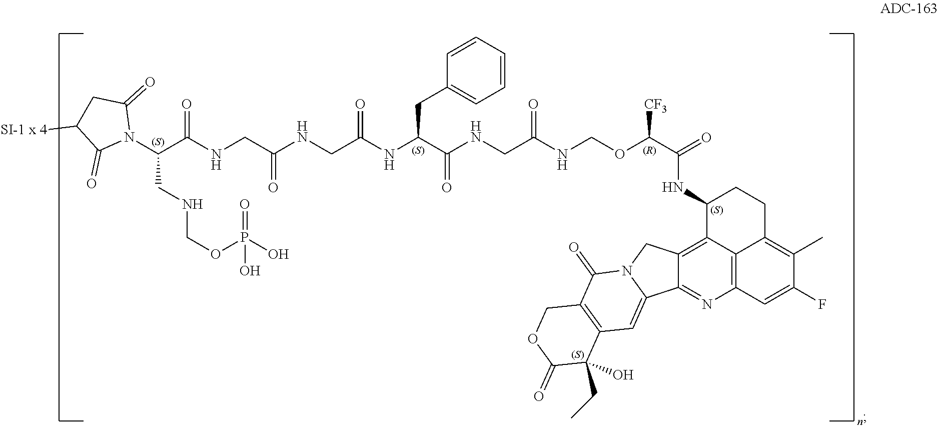

### Example 259

ADC-164 was prepared according to the general-purpose coupling method of Example 94,

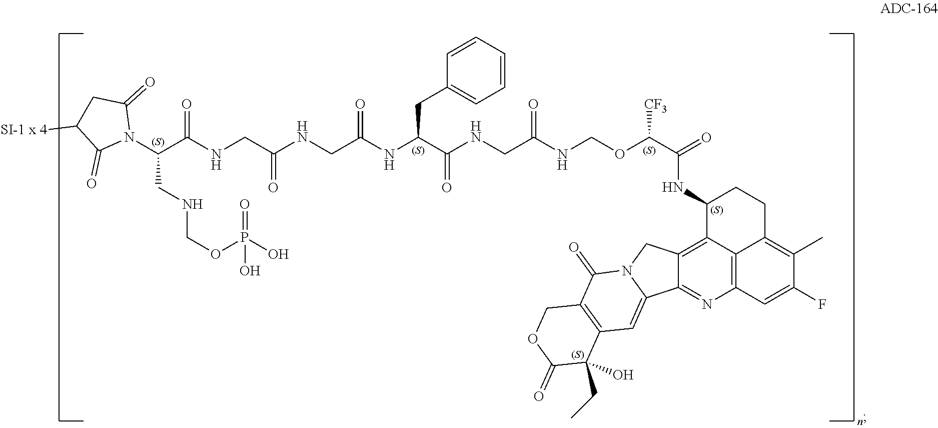

### Example 260

ADC-165 was prepared according to the general-purpose coupling method of Example 94,

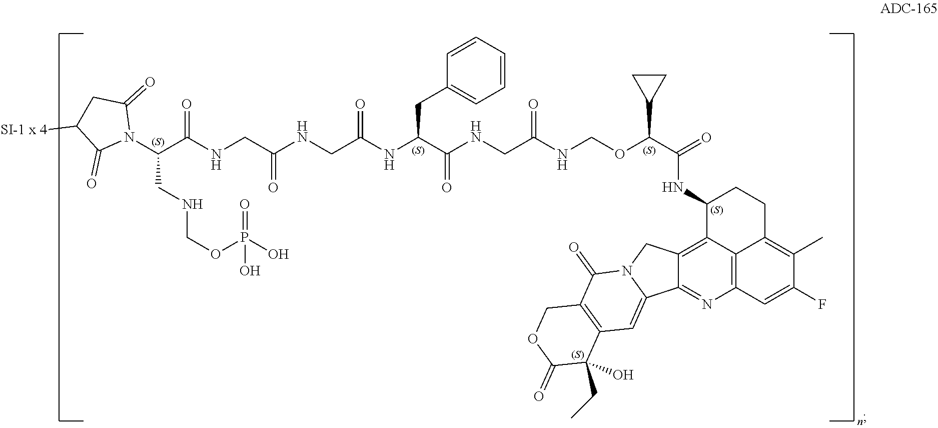

### Example 261

ADC-166 was prepared according to the general-purpose coupling method of Example 94,

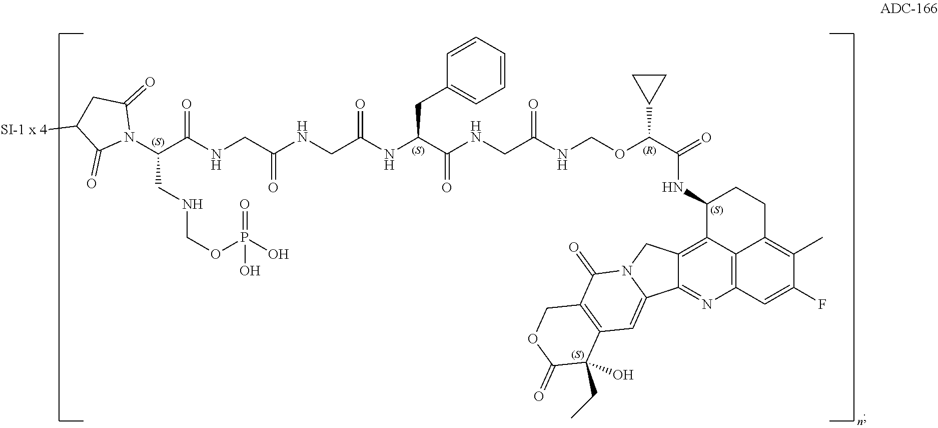

### Example 262

ADC-167 was prepared according to the general-purpose coupling method of Example 94,

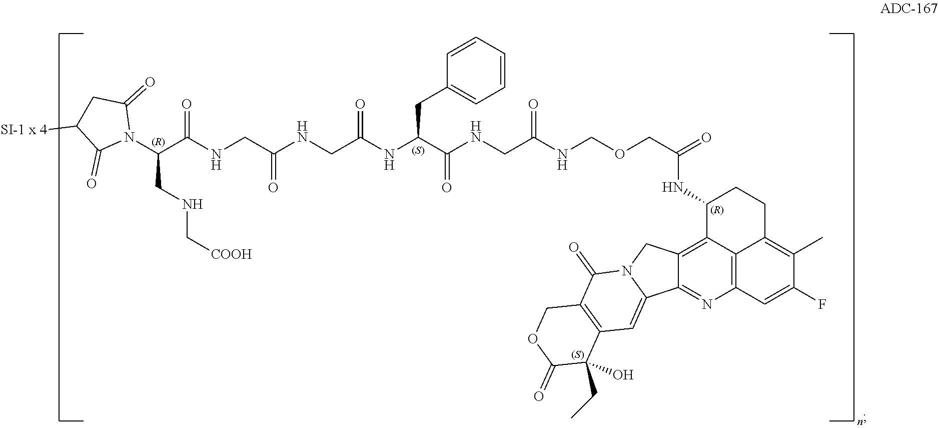

### Example 263

ADC-168 was prepared according to the general-purpose coupling method of Example 94,

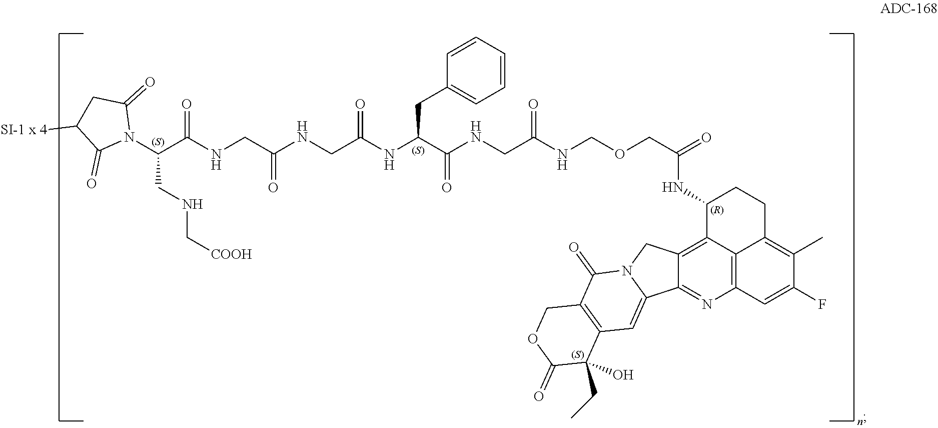

### Example 264

ADC-169 was prepared according to the general-purpose coupling method of Example 94,

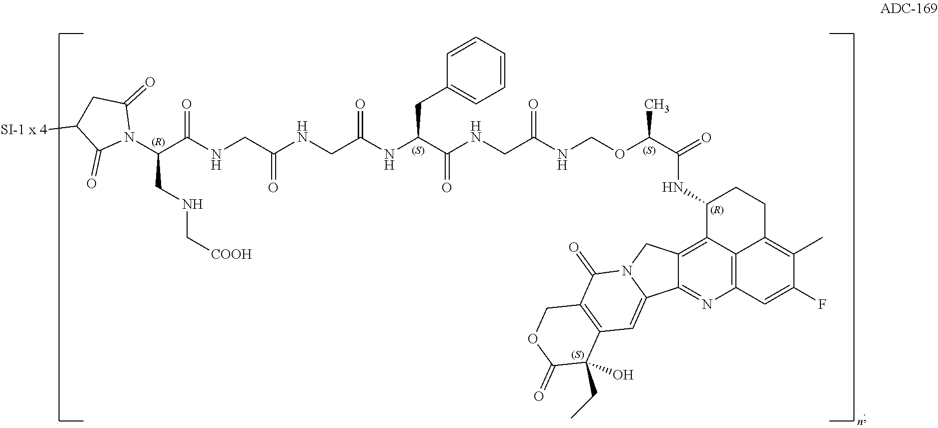

### Example 265

ADC-170 was prepared according to the general-purpose coupling method of Example 94,

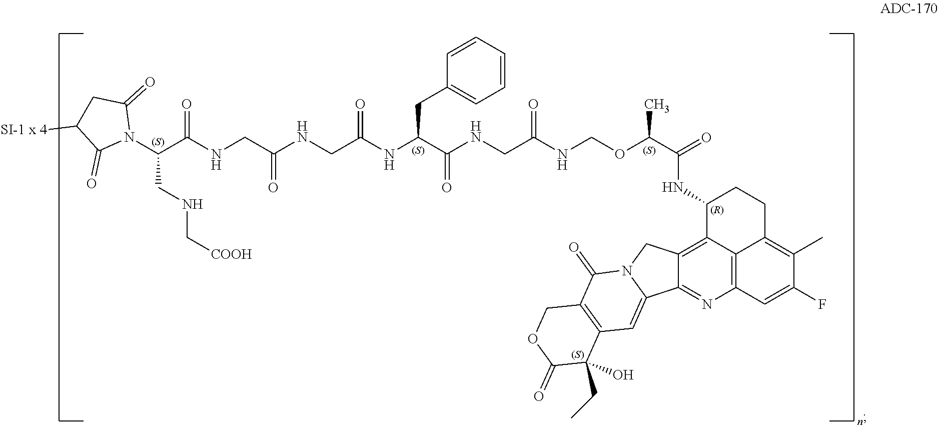

### Example 266

ADC-171 was prepared according to the general-purpose coupling method of Example 94,

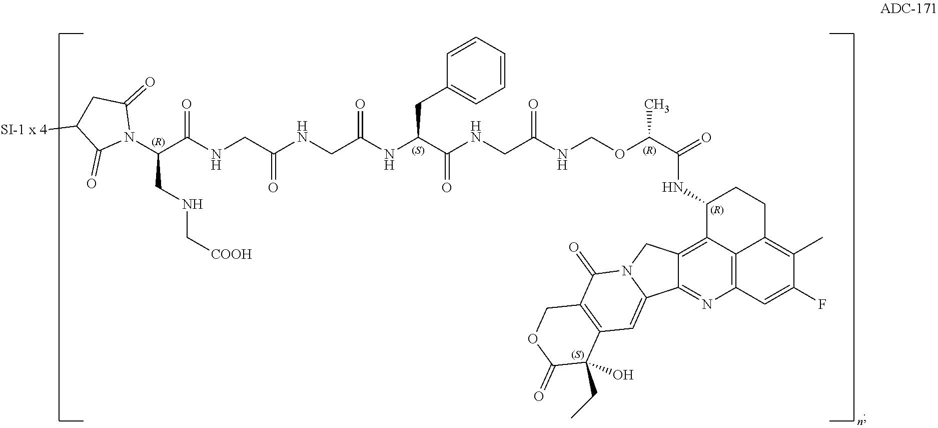

### Example 267

ADC-172 was prepared according to the general-purpose coupling method of Example 94,

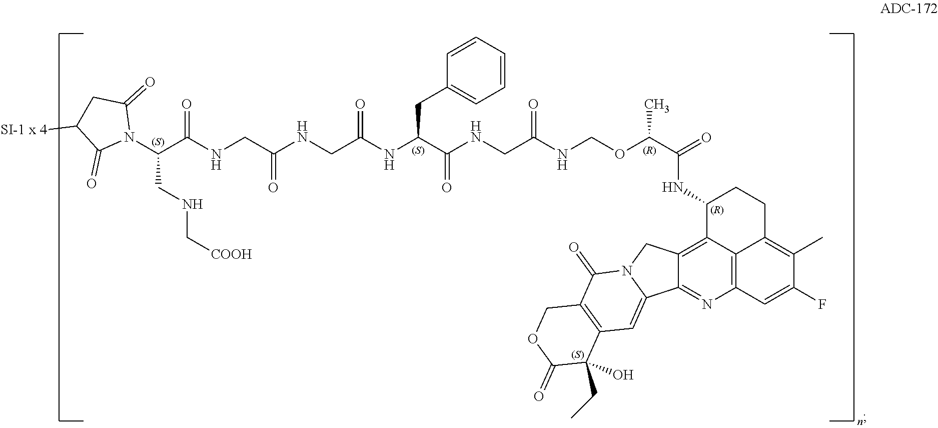

### Example 268

ADC-173 was prepared according to the general-purpose coupling method of Example 94,

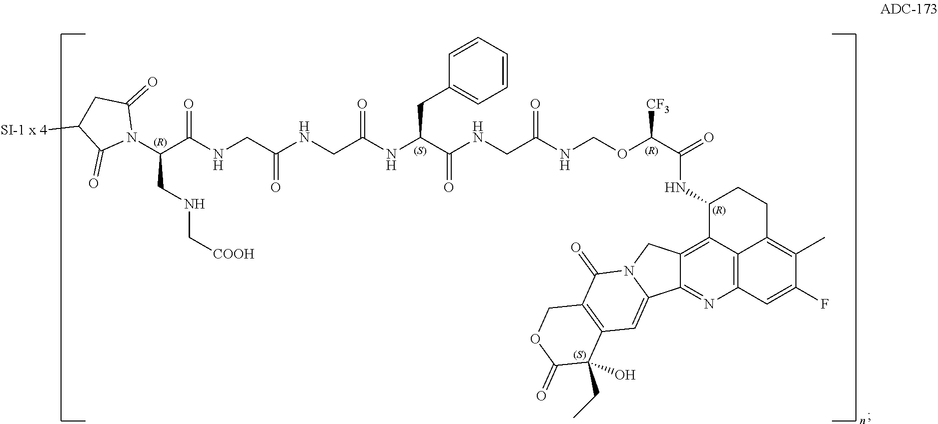

### Example 269

ADC-174 was prepared according to the general-purpose coupling method of Example 94,

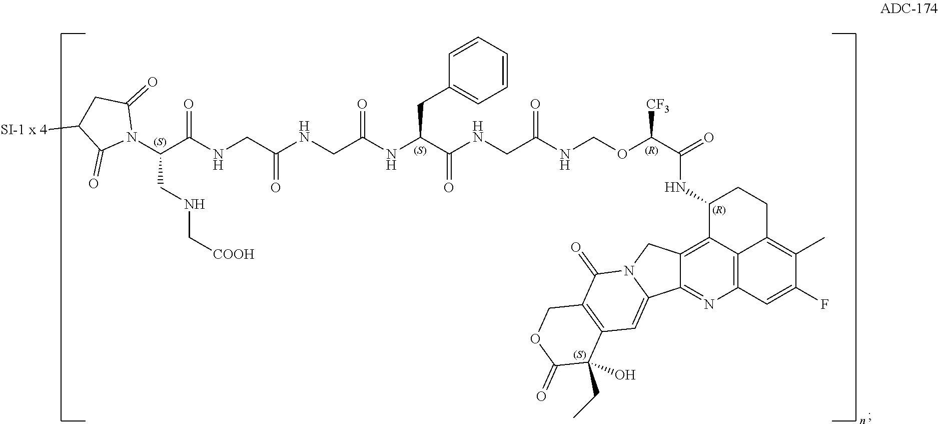

### Example 270

ADC-175 was prepared according to the general-purpose coupling method of Example 94,

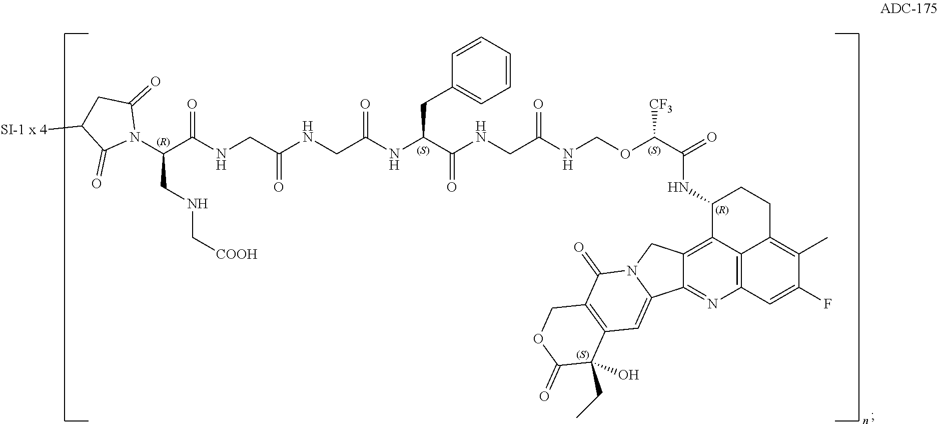

### Example 271

ADC-176 was prepared according to the general-purpose coupling method of Example 94,

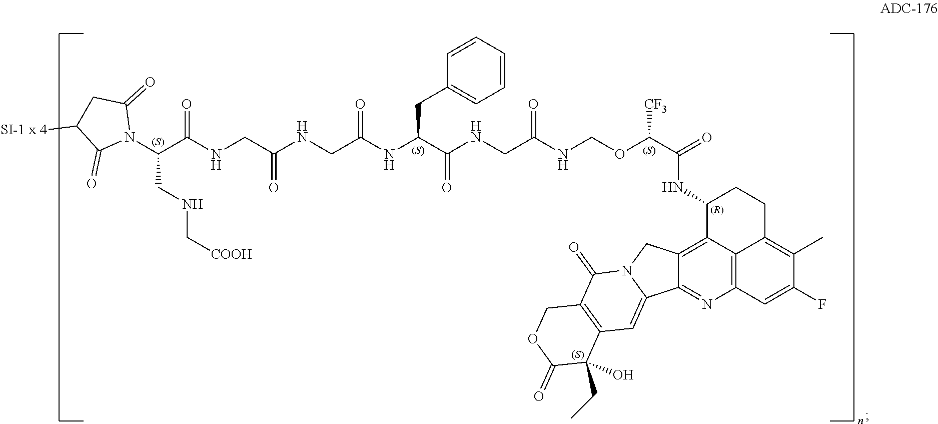

### Example 272

ADC-177 was prepared according to the general-purpose coupling method of Example 94,

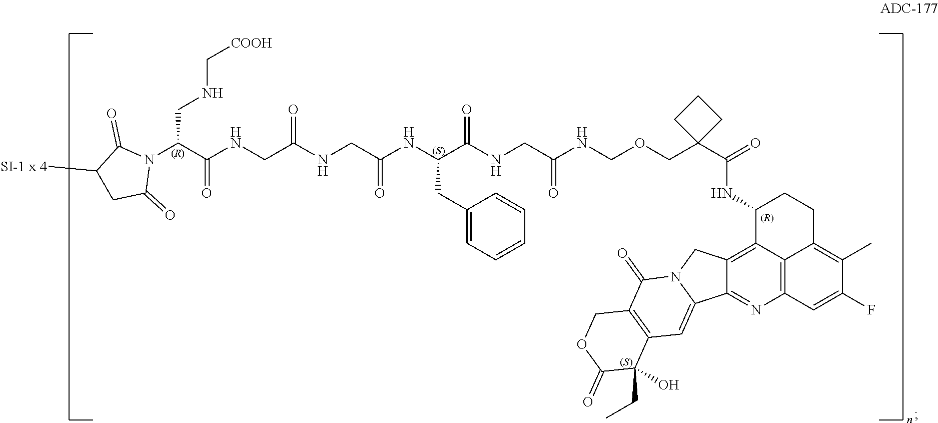

### Example 273

ADC-178 was prepared according to the general-purpose coupling method of Example 94,

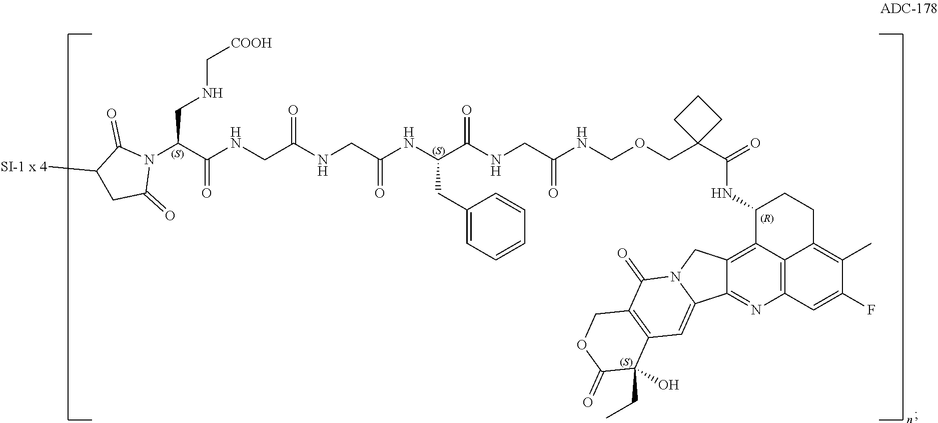

### Example 274

ADC-179 was prepared according to the general-purpose coupling method of Example 94,

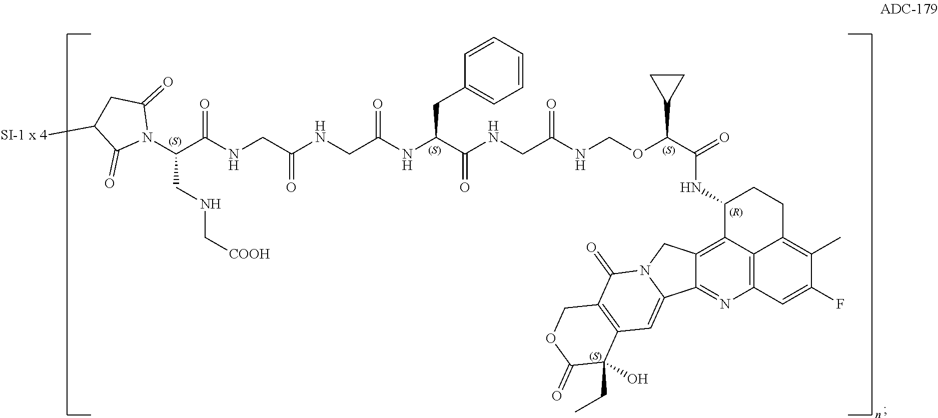

### Example 275

ADC-180 was prepared according to the general-purpose coupling method of Example 94,

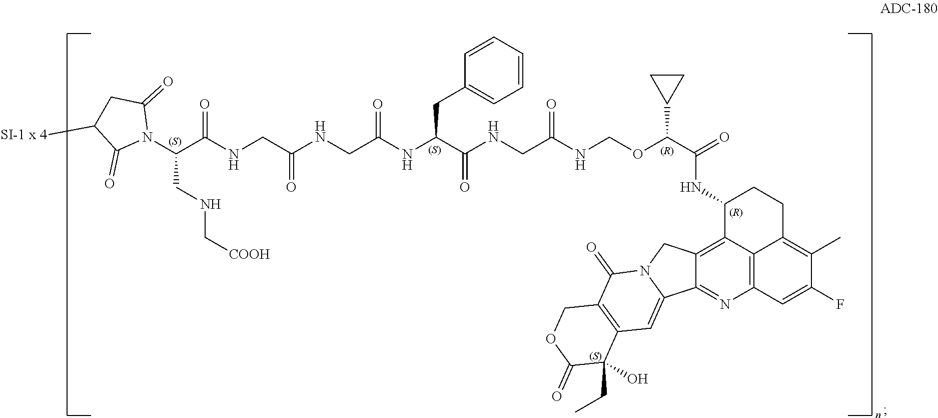

### Example 276

ADC-181 was prepared according to the general-purpose coupling method of Example 94,

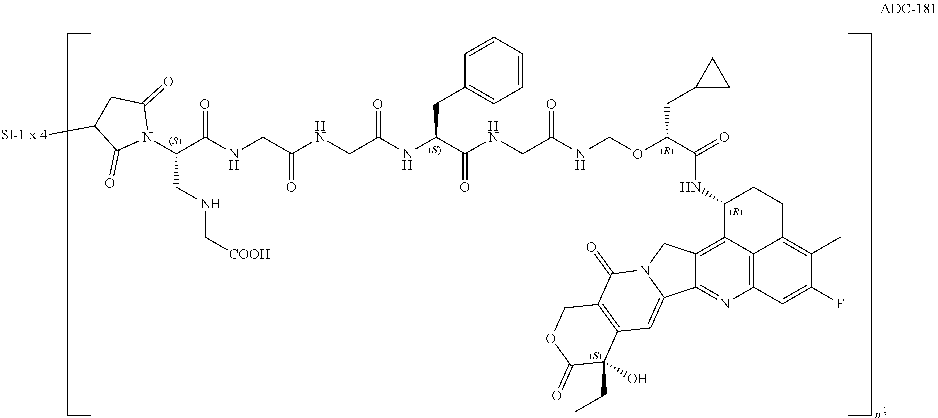

### Example 277

ADC-182 was prepared according to the general-purpose coupling method of Example 94,

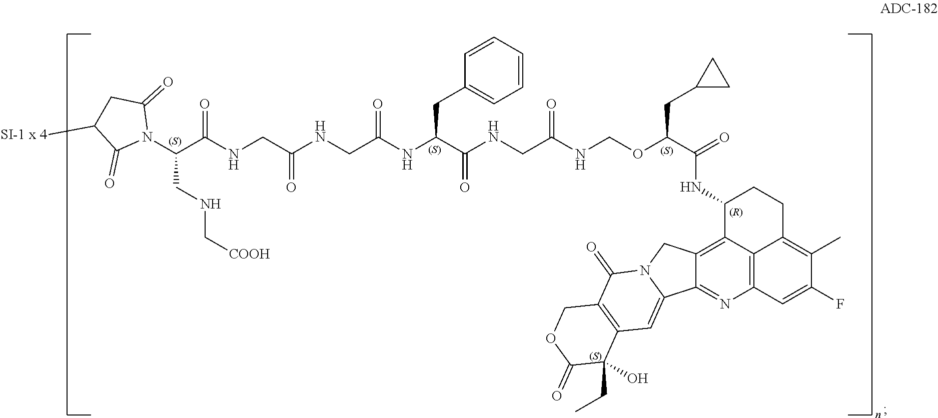

### Example 278

ADC-183 was prepared according to the general-purpose coupling method of Example 94,

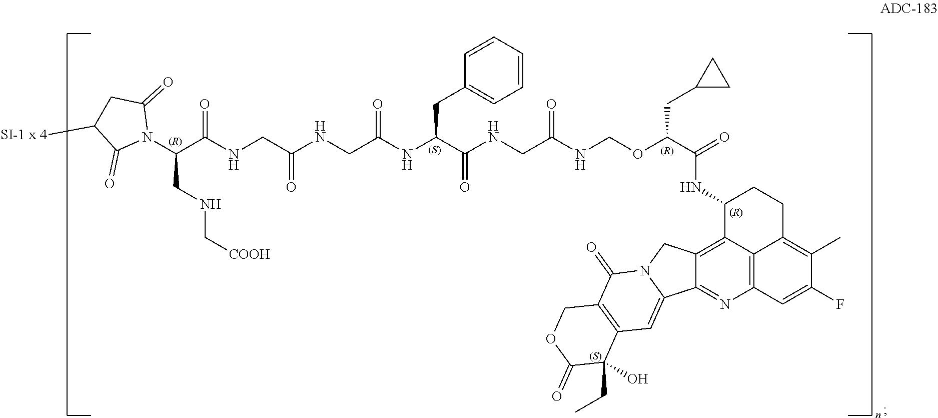

### Example 279

ADC-184 was prepared according to the general-purpose coupling method of Example 94,

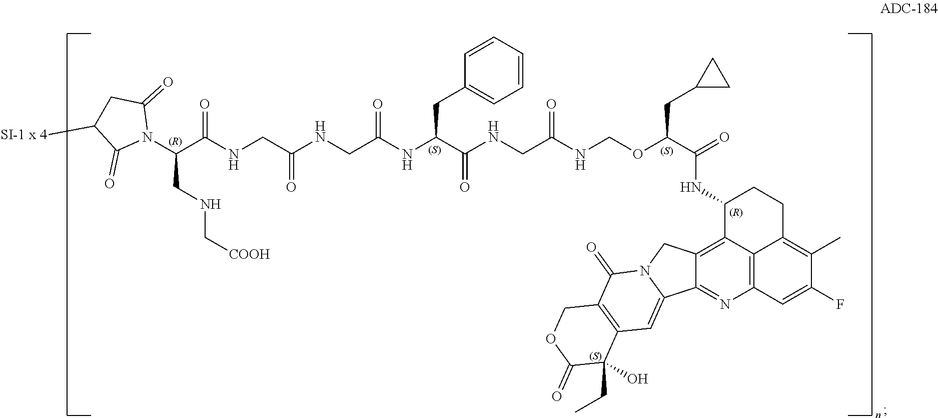

### Example 280

ADC-185 was prepared according to the general-purpose coupling method of Example 94,

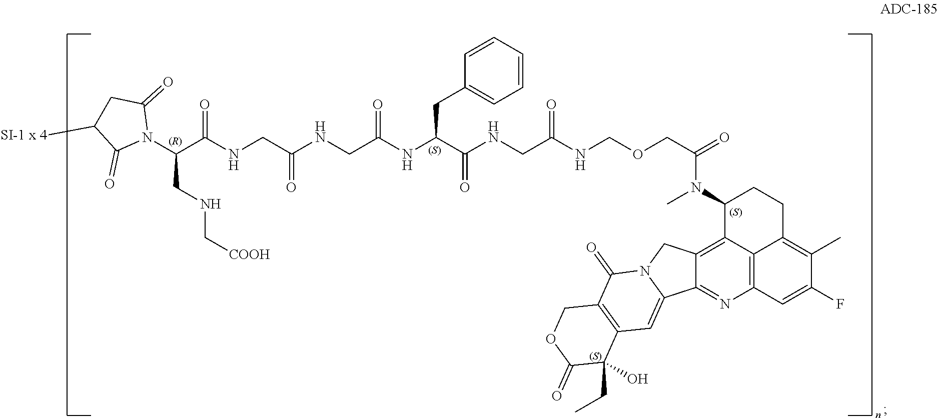

### Example 281

ADC-186 was prepared according to the general-purpose coupling method of Example 94,

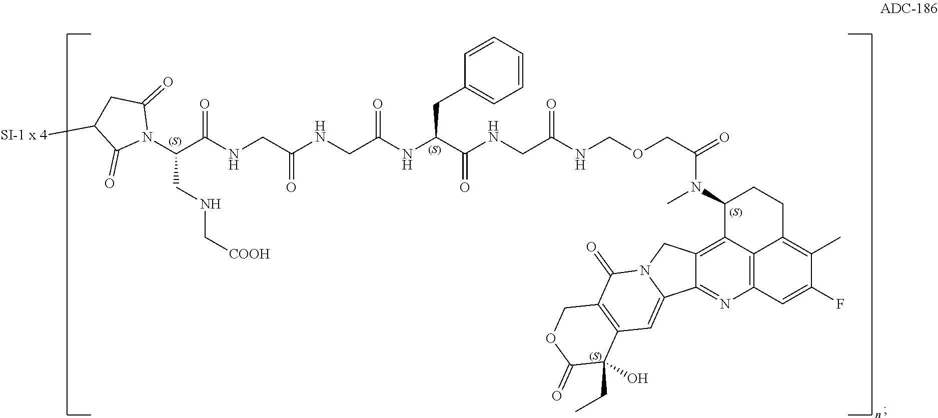

### Example 282

ADC-187 was prepared according to the general-purpose coupling method of Example 94,

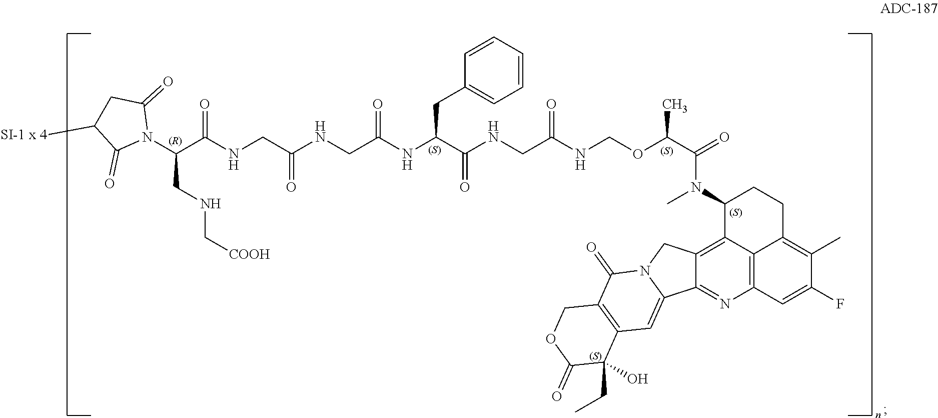

### Example 283

ADC-188 was prepared according to the general-purpose coupling method of Example 94,

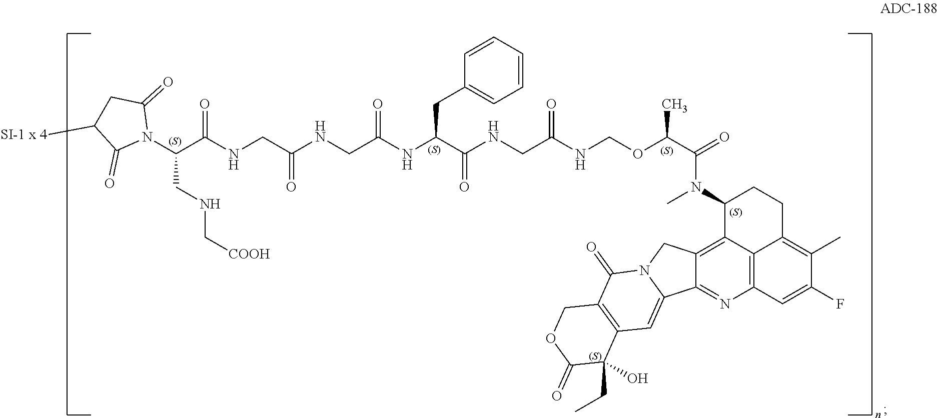

### Example 284

ADC-189 was prepared according to the general-purpose coupling method of Example 94,

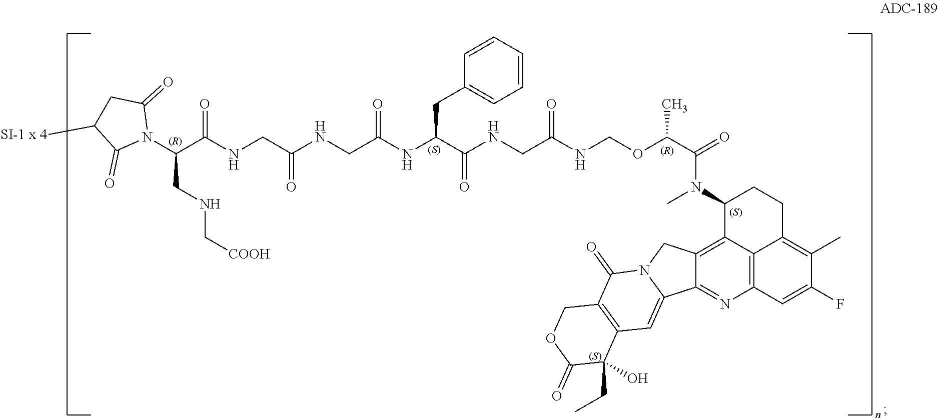

### Example 285

ADC-190 was prepared according to the general-purpose coupling method of Example 94,

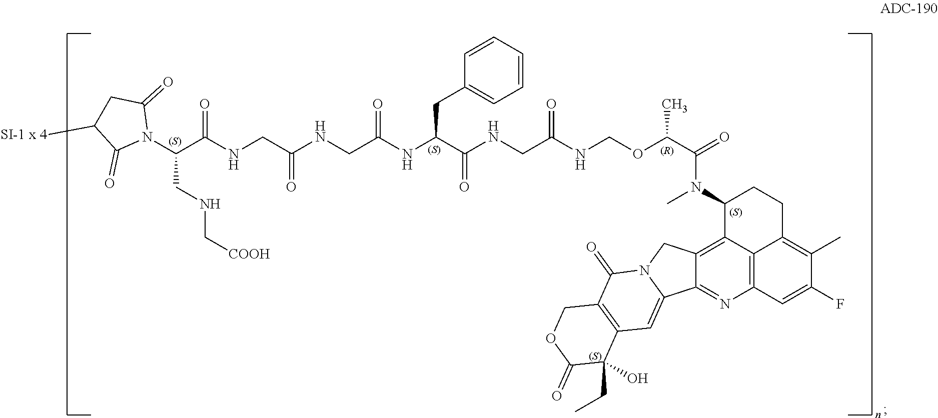

### Example 286

ADC-191 was prepared according to the general-purpose coupling method of Example 94,

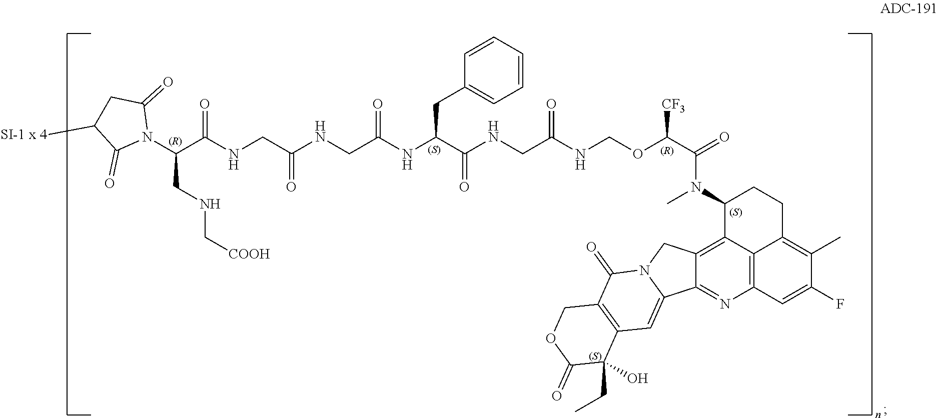

### Example 287

ADC-192 was prepared according to the general-purpose coupling method of Example 94,

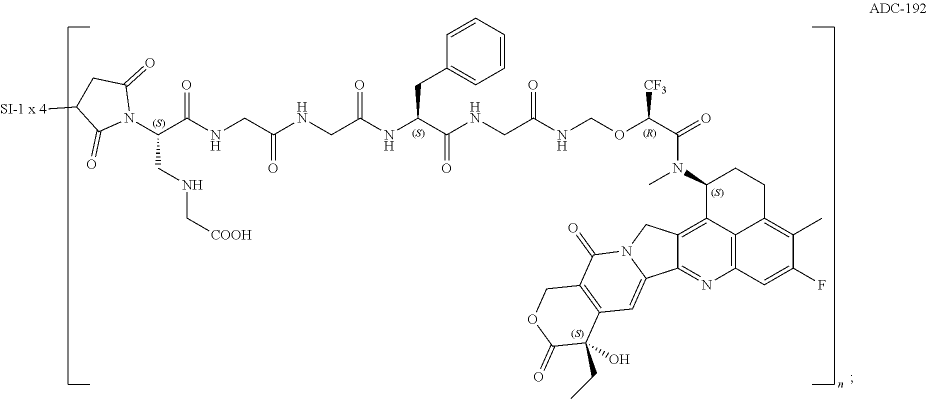

### Example 288

ADC-193 was prepared according to the general-purpose coupling method of Example 94,

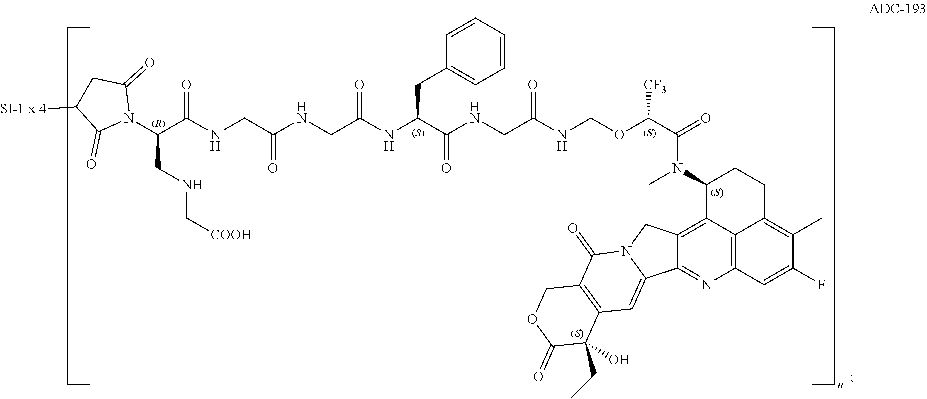

### Example 289

ADC-194 was prepared according to the general-purpose coupling method of Example 94,

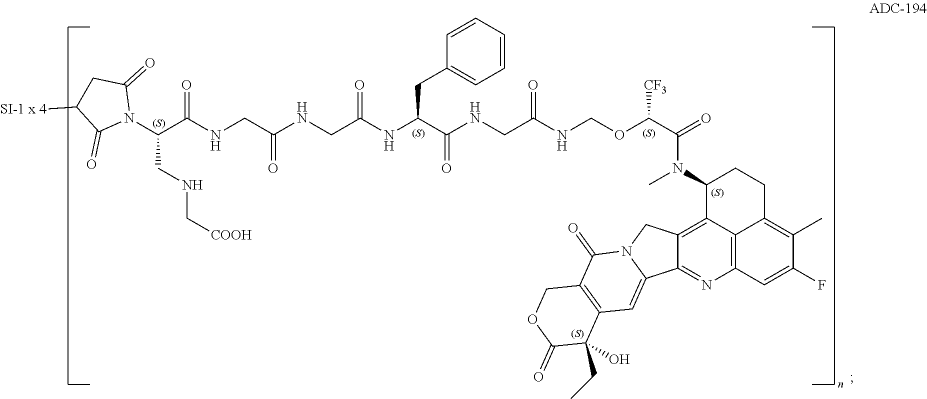

### Example 290

ADC-195 was prepared according to the general-purpose coupling method of Example 94,

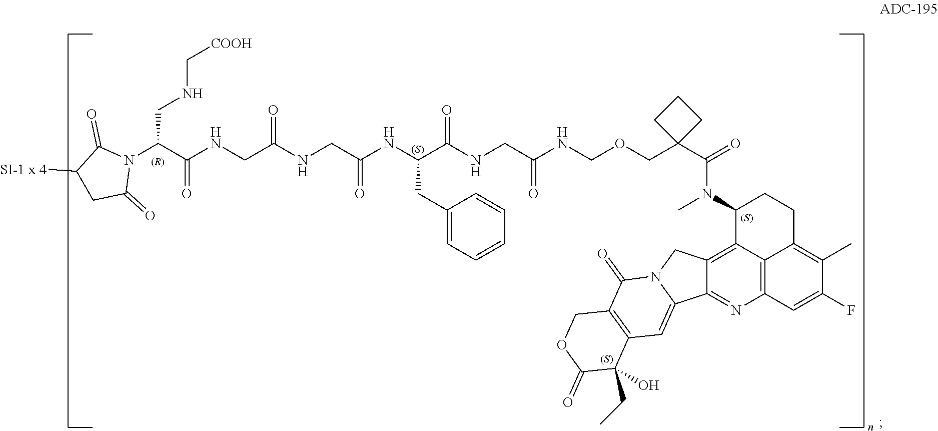

### Example 291

ADC-196 was prepared according to the general-purpose coupling method of Example 94,

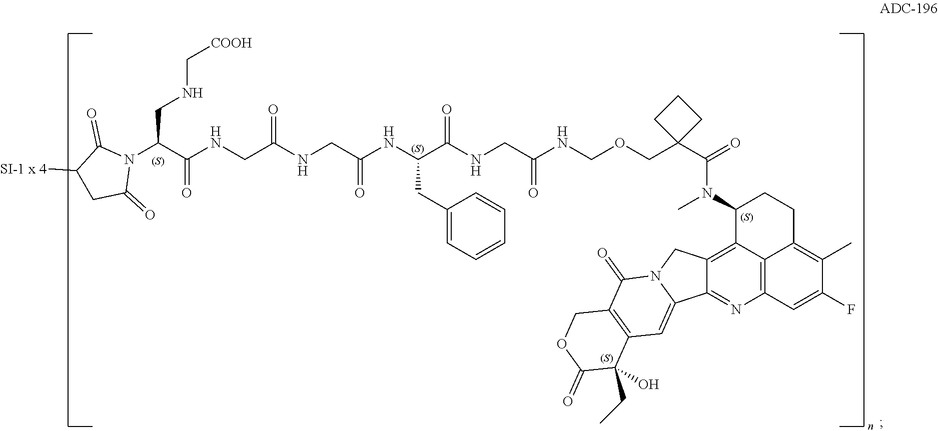

### Example 292

ADC-197 was prepared according to the general-purpose coupling method of Example 94,

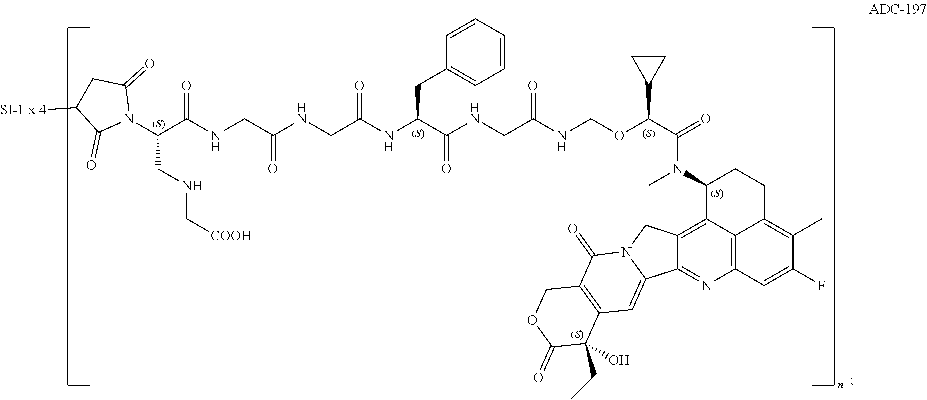

### Example 293

ADC-198 was prepared according to the general-purpose coupling method of Example 94,

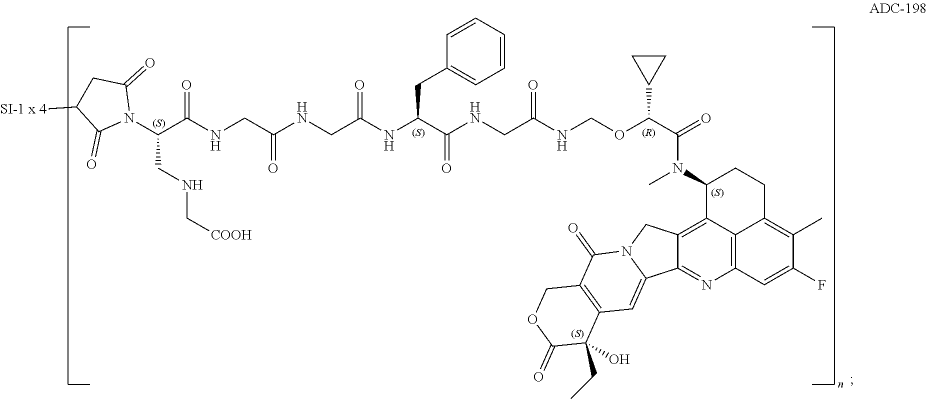

### Example 294

ADC-199 was prepared according to the general-purpose coupling method of Example 94,

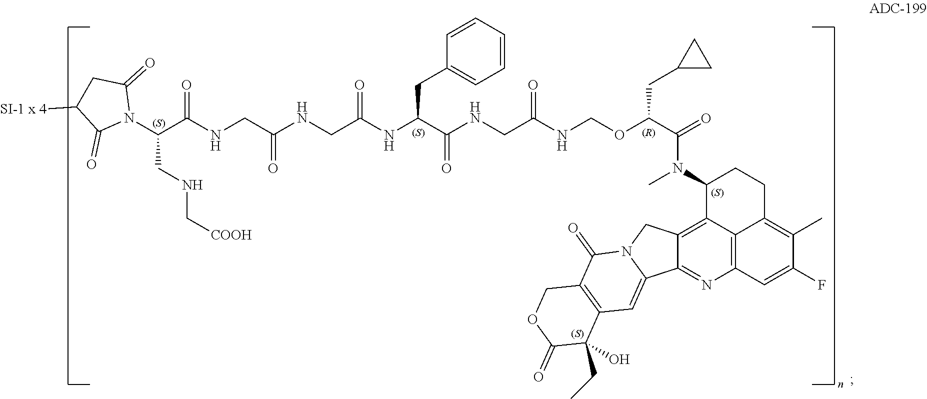

### Example 295

ADC-200 was prepared according to the general-purpose coupling method of Example 94,

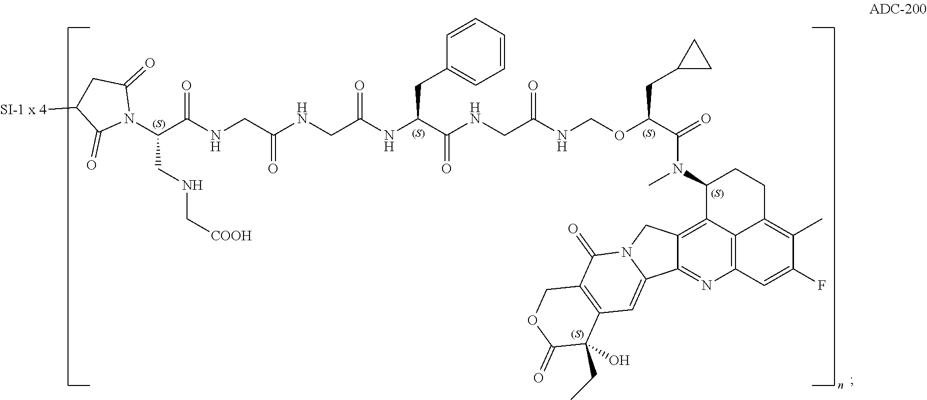

### Example 296

ADC-201 was prepared according to the general-purpose coupling method of Example 94,

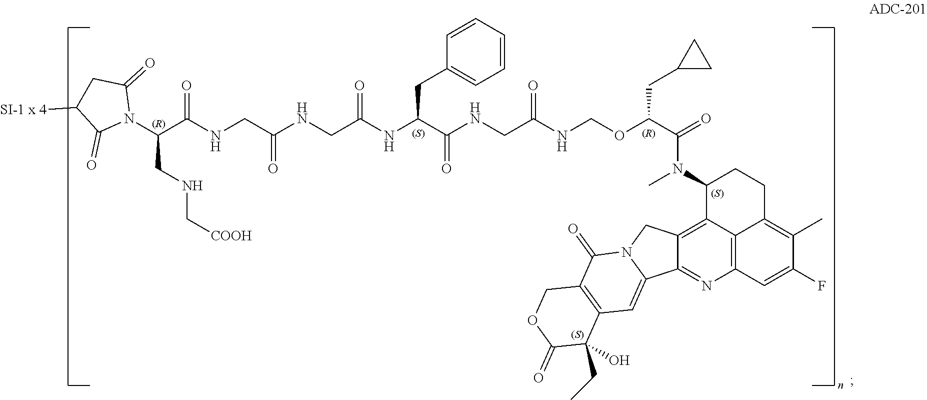

### Example 297

ADC-202 was prepared according to the general-purpose coupling method of Example 94,

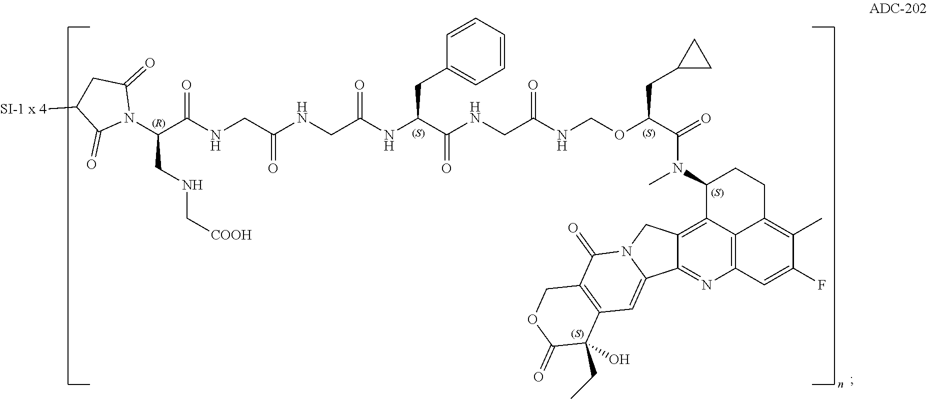

### Example 298

ADC-203 was prepared according to the general-purpose coupling method of Example 94,

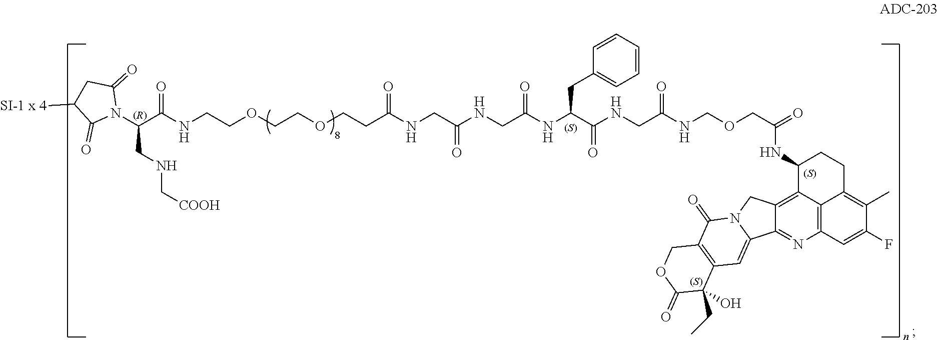

### Example 299

ADC-204 was prepared according to the general-purpose coupling method of Example 94,

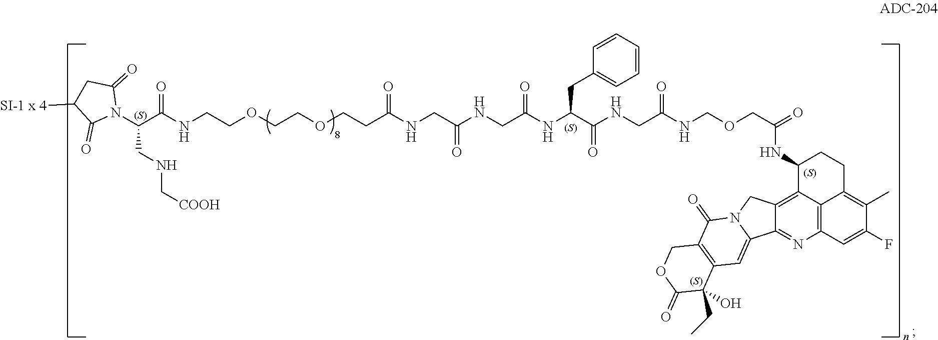

### Example 300

ADC-205 was prepared according to the general-purpose coupling method of Example 94,

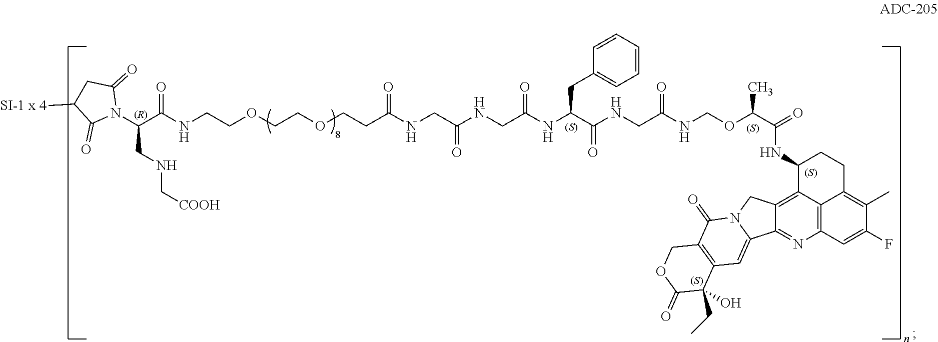

### Example 301

ADC-206 was prepared according to the general-purpose coupling method of Example 94,

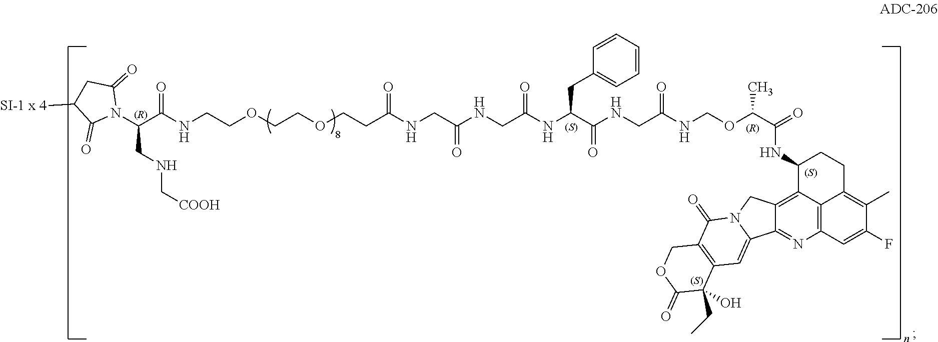

### Example 302

ADC-207 was prepared according to the general-purpose coupling method of Example 94,

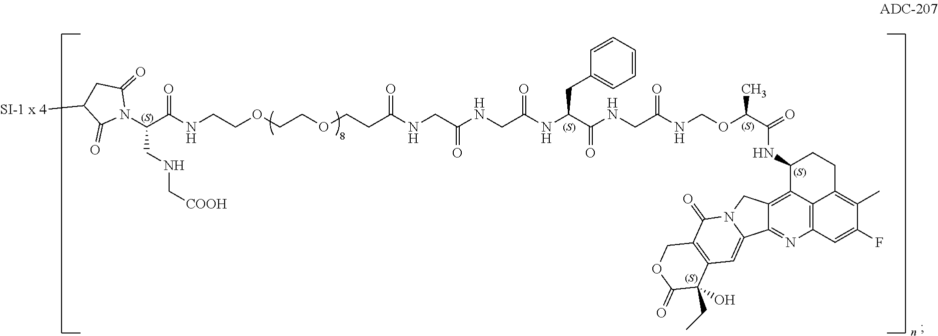

### Example 303

ADC-208 was prepared according to the general-purpose coupling method of Example 94,

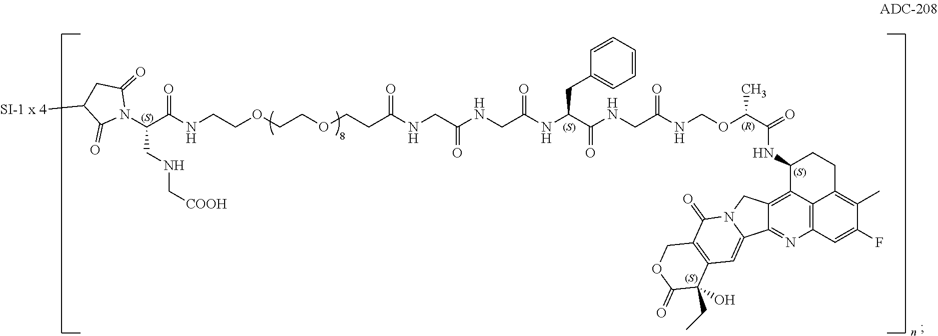

### Example 304

ADC-209 was prepared according to the general-purpose coupling method of Example 94,

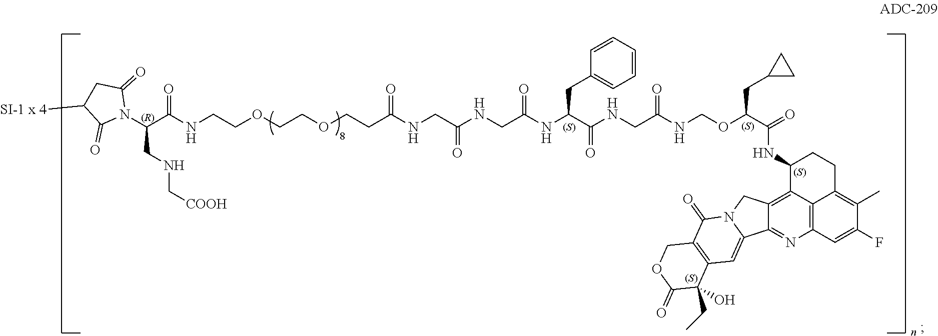

### Example 305

ADC-210 was prepared according to the general-purpose coupling method of Example 94,

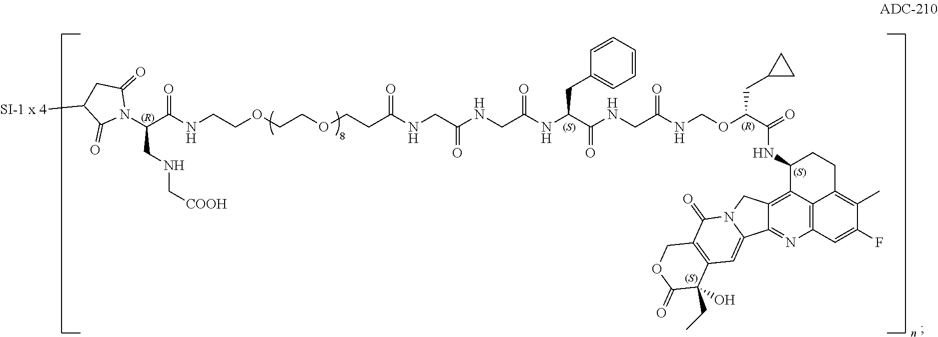

### Example 306

ADC-211 was prepared according to the general-purpose coupling method of Example 94,

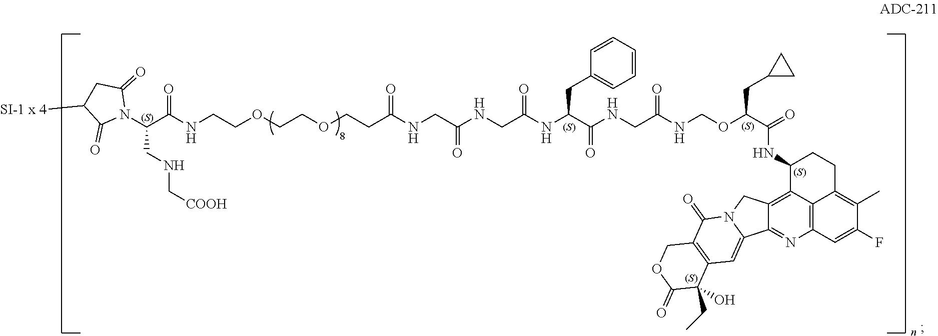

### Example 307

ADC-212 was prepared according to the general-purpose coupling method of Example 94,

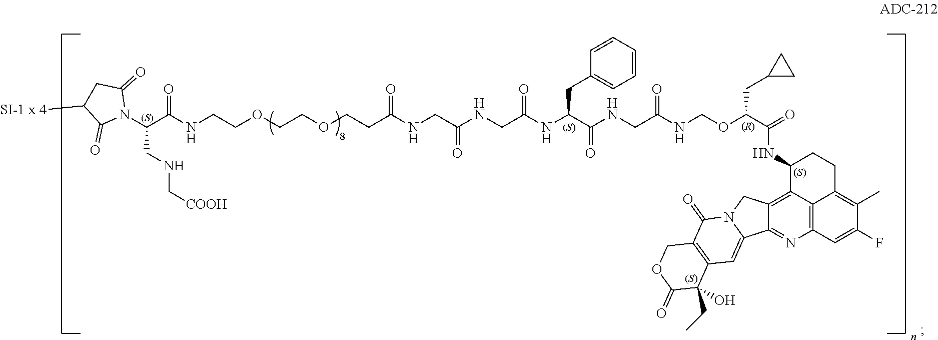

### Example 308

ADC-213 was prepared from compound 45 according to the general-purpose coupling method of Example 94,

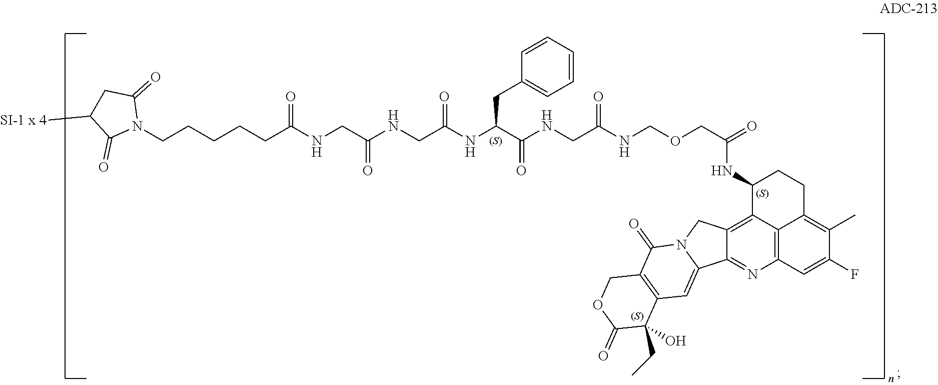

### Example 309

ADC-214 was prepared from compound 5A according to the general-purpose coupling method of Example 93,

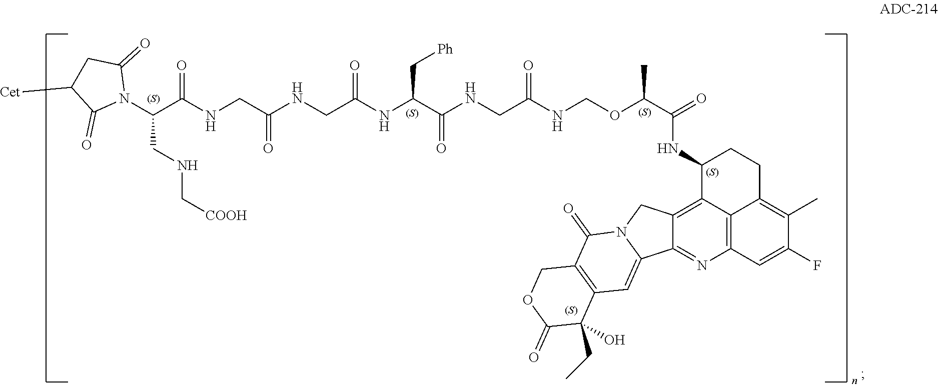

### Example 310

ADC-215 of HER3 was prepared from compound 5A according to the general-purpose coupling method of Example 93,

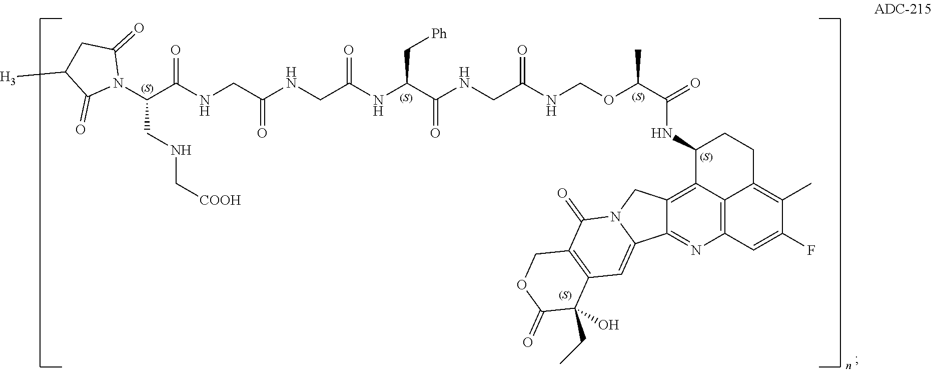

where the H3 antibody is the portion of the SI-1×6.4 antibody knockout targeting EGFR.

### Example 311

ADC-216 to ADC-223 were prepared according to the general-purpose coupling method of Example 93,

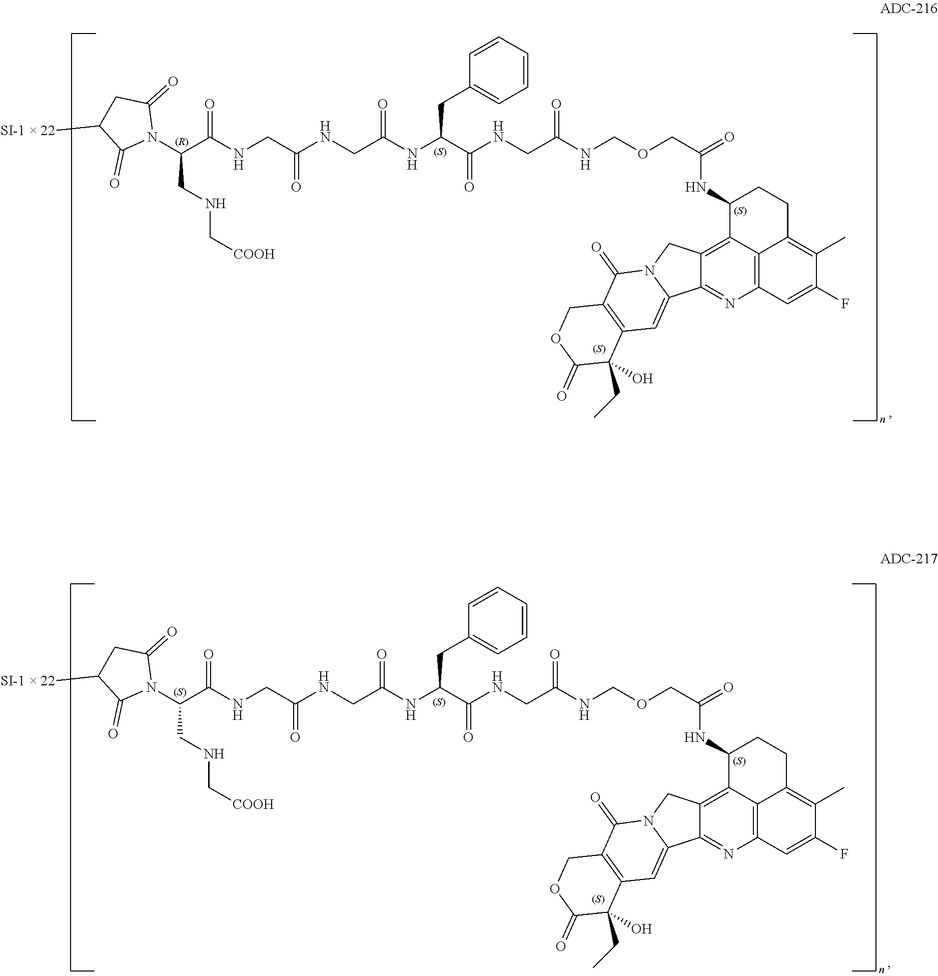

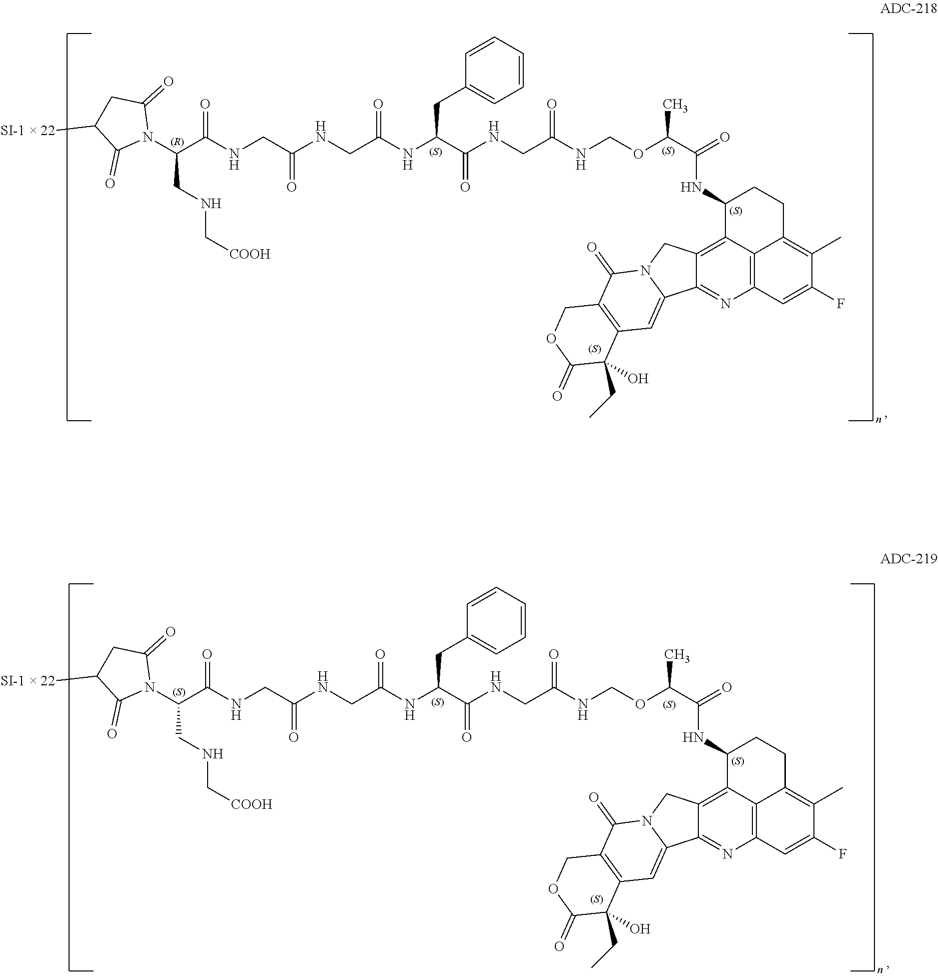

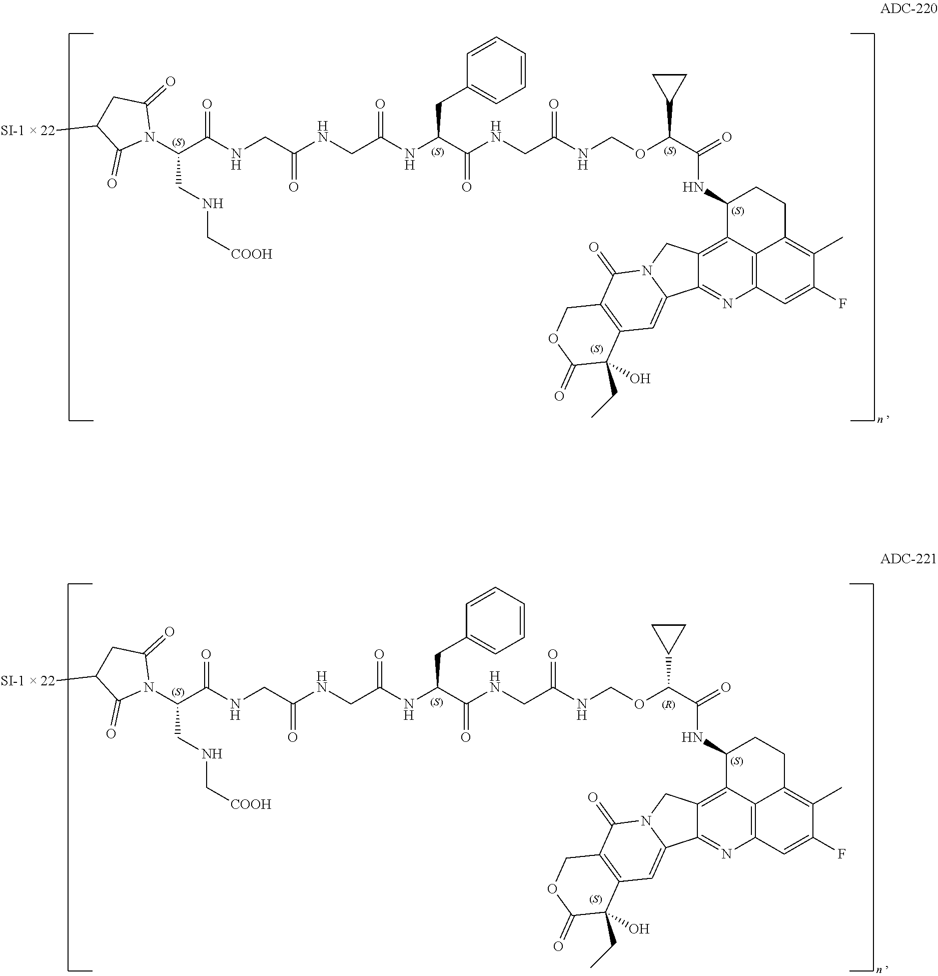

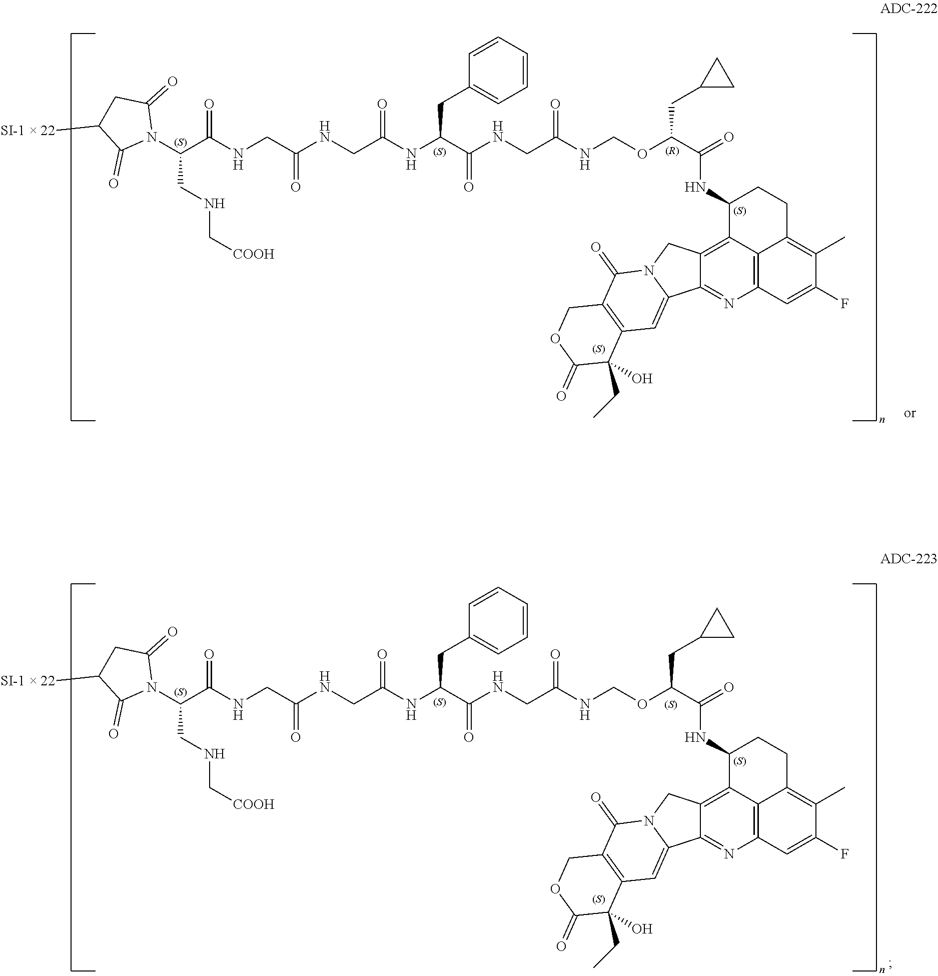

### Example 312

ADC-224 to ADC-231 were prepared according to the general-purpose coupling method of Example 93,

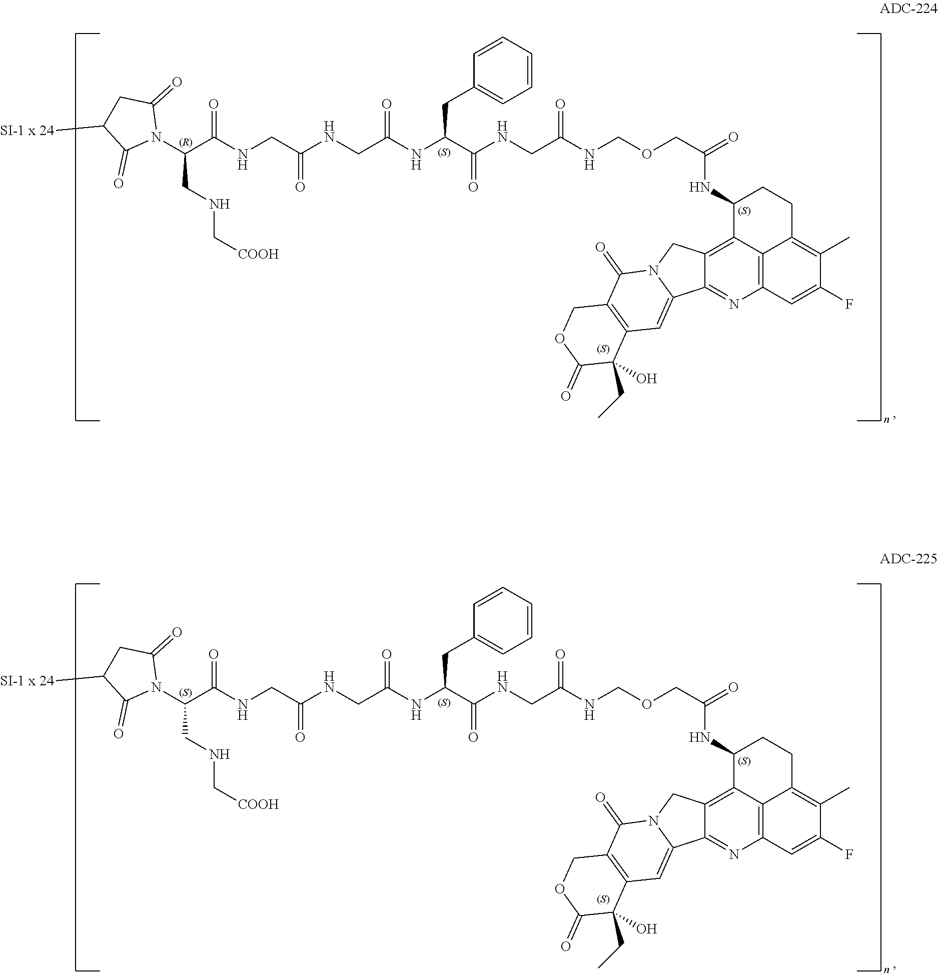

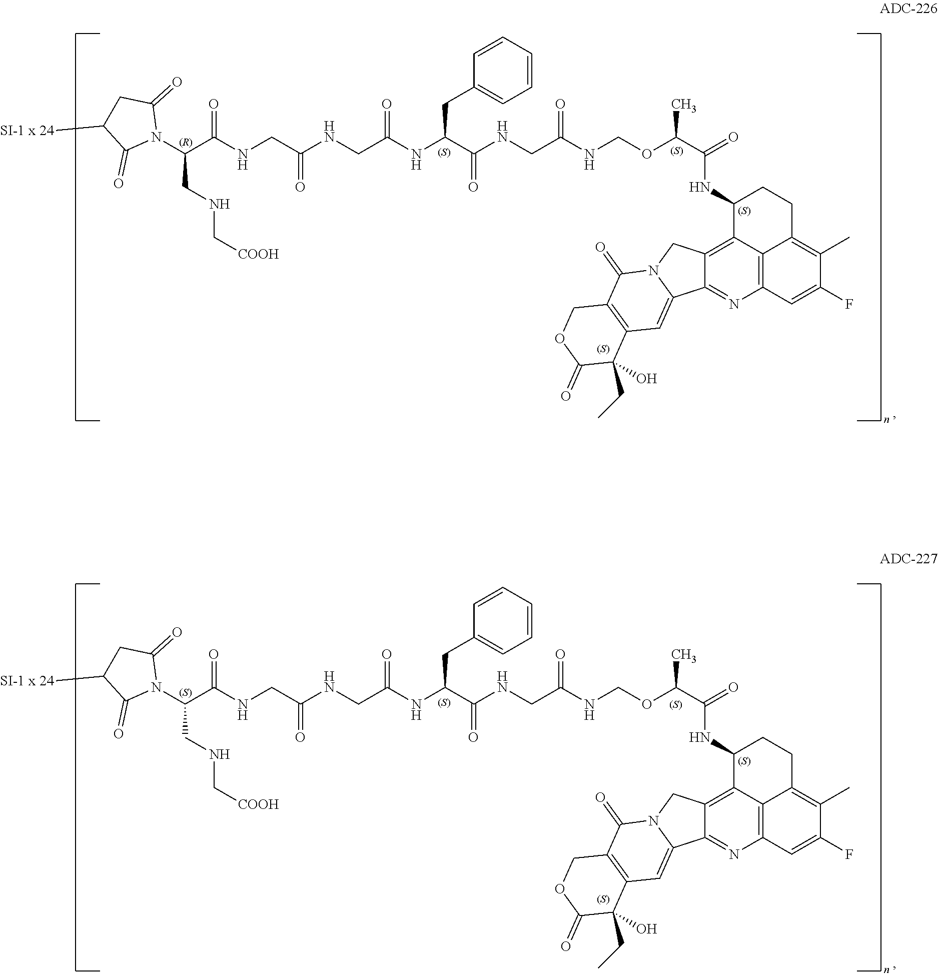

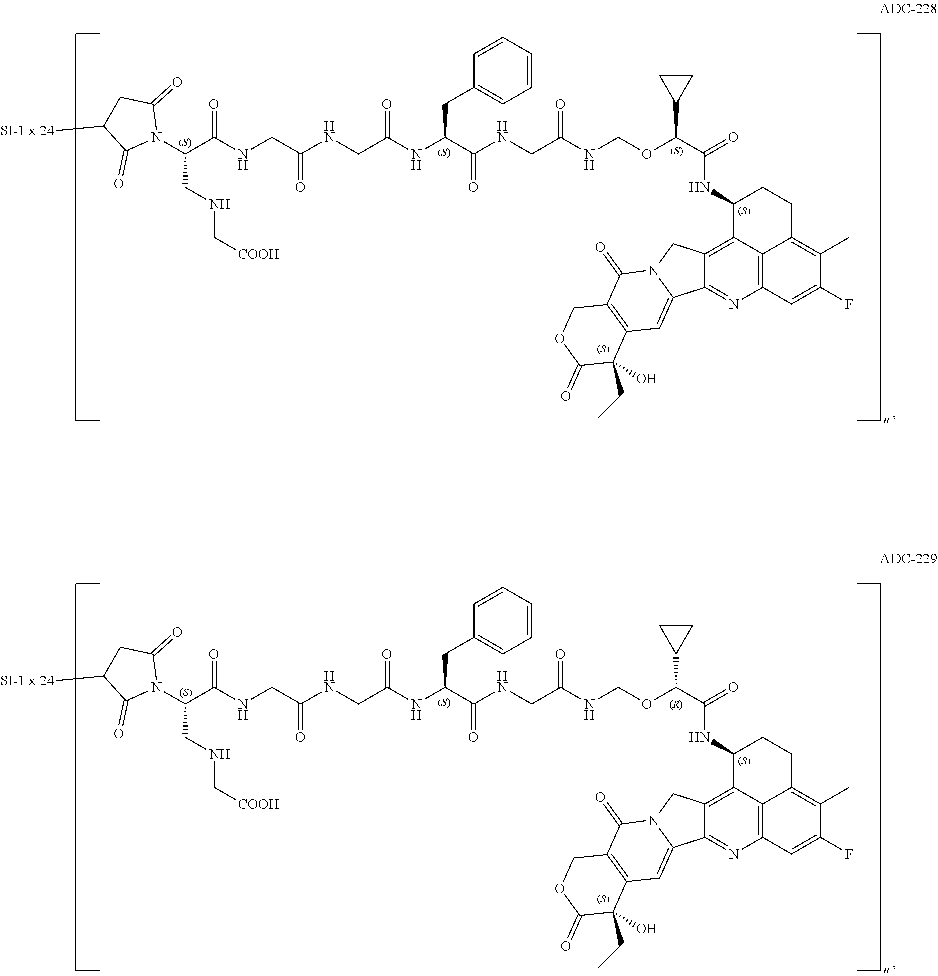

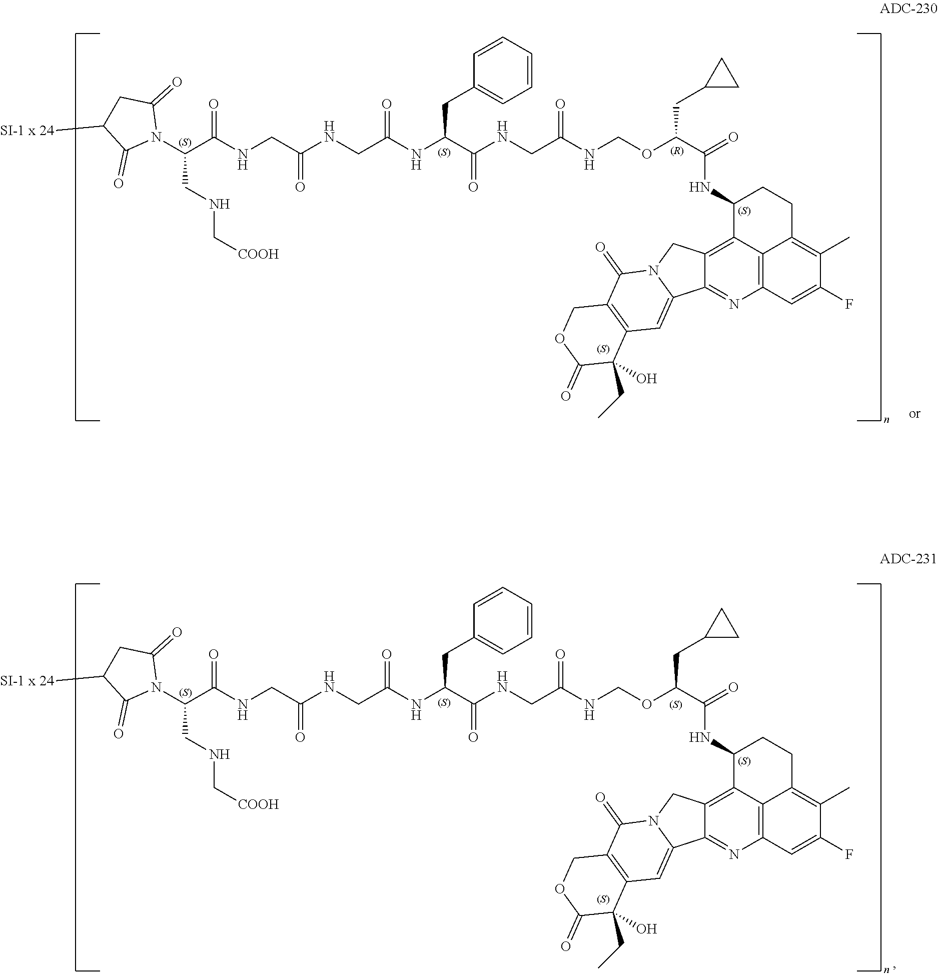

### Example 313

ADC-232 to ADC-239 were prepared according to the general-purpose coupling method of Example 94,

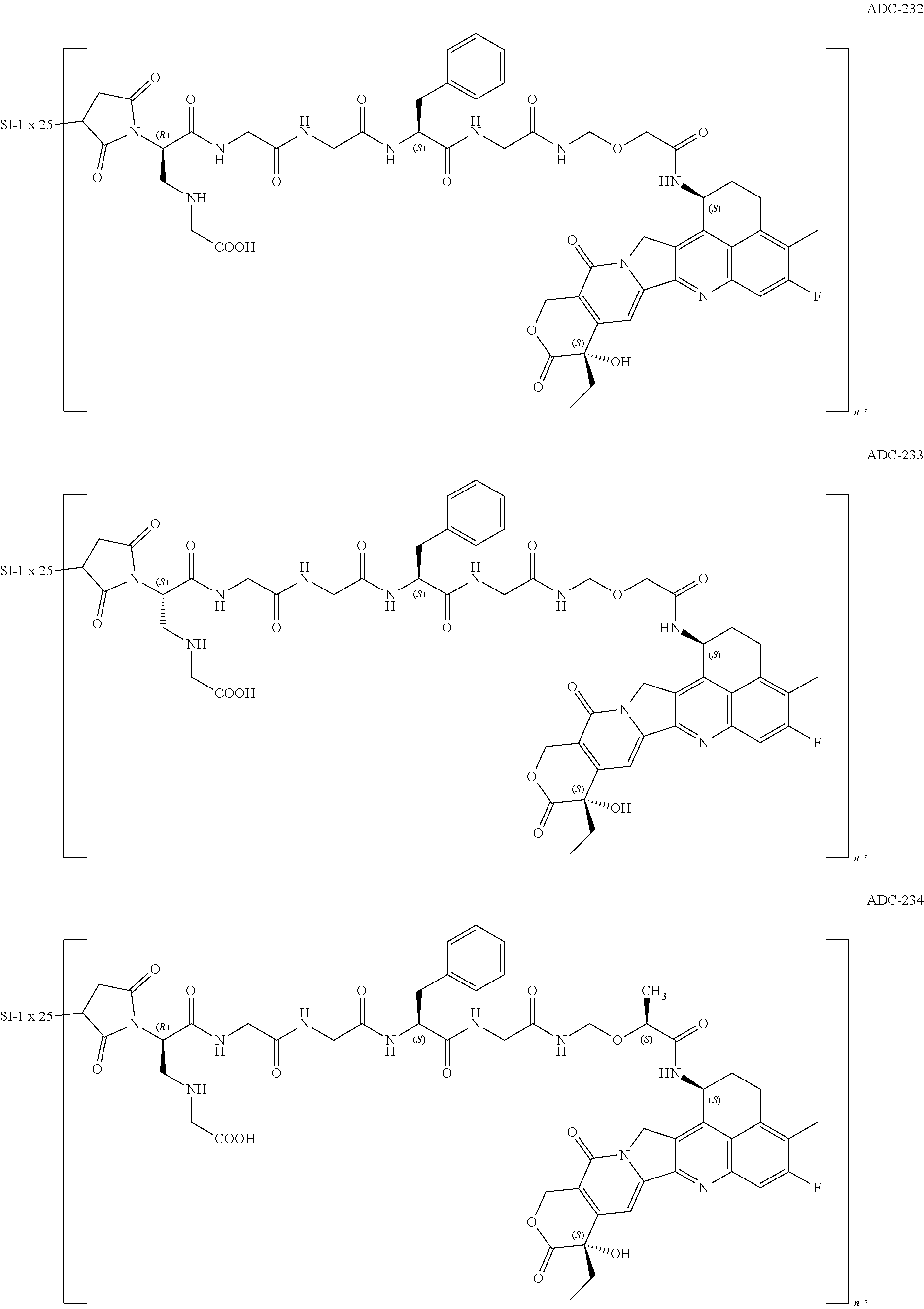

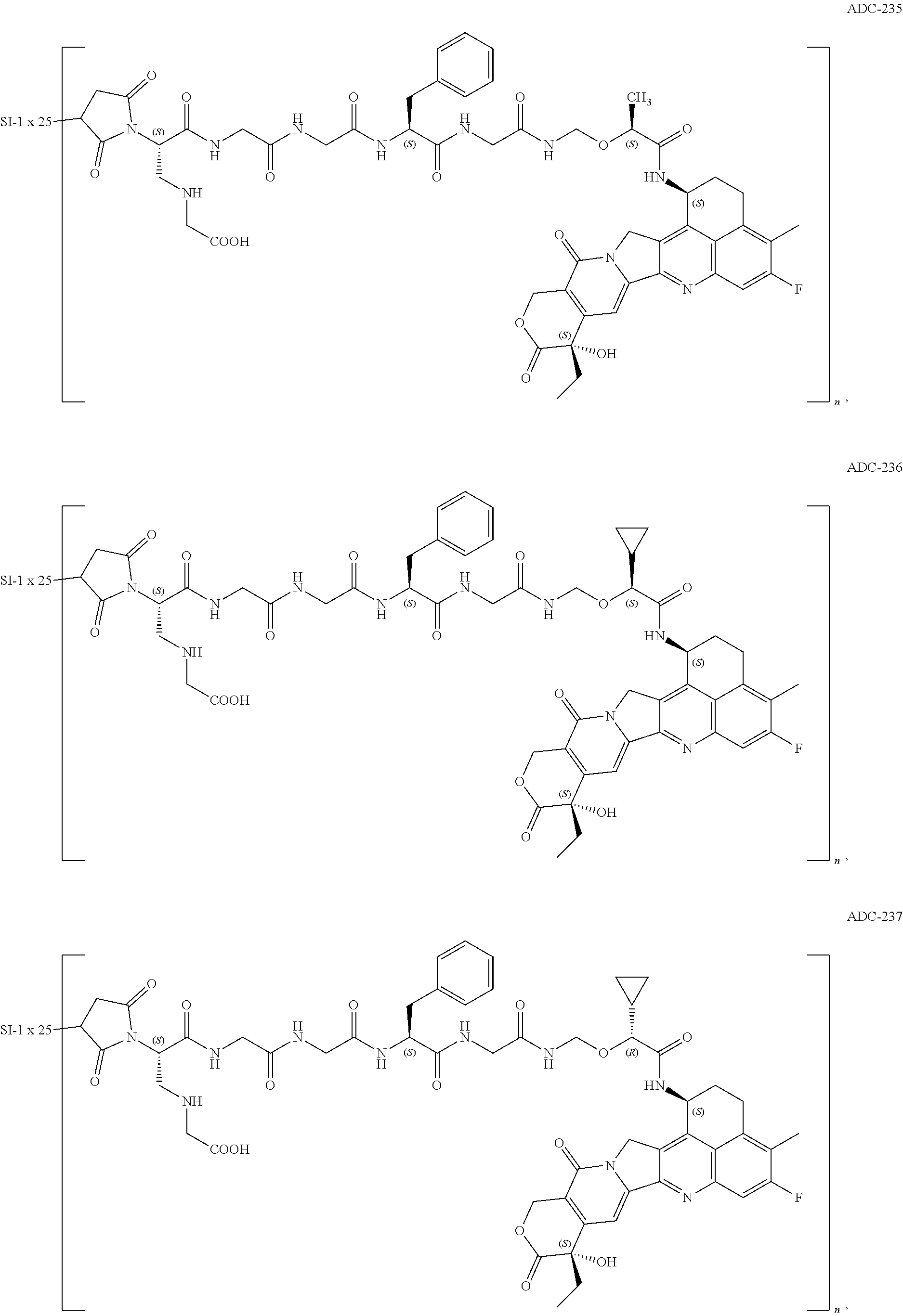

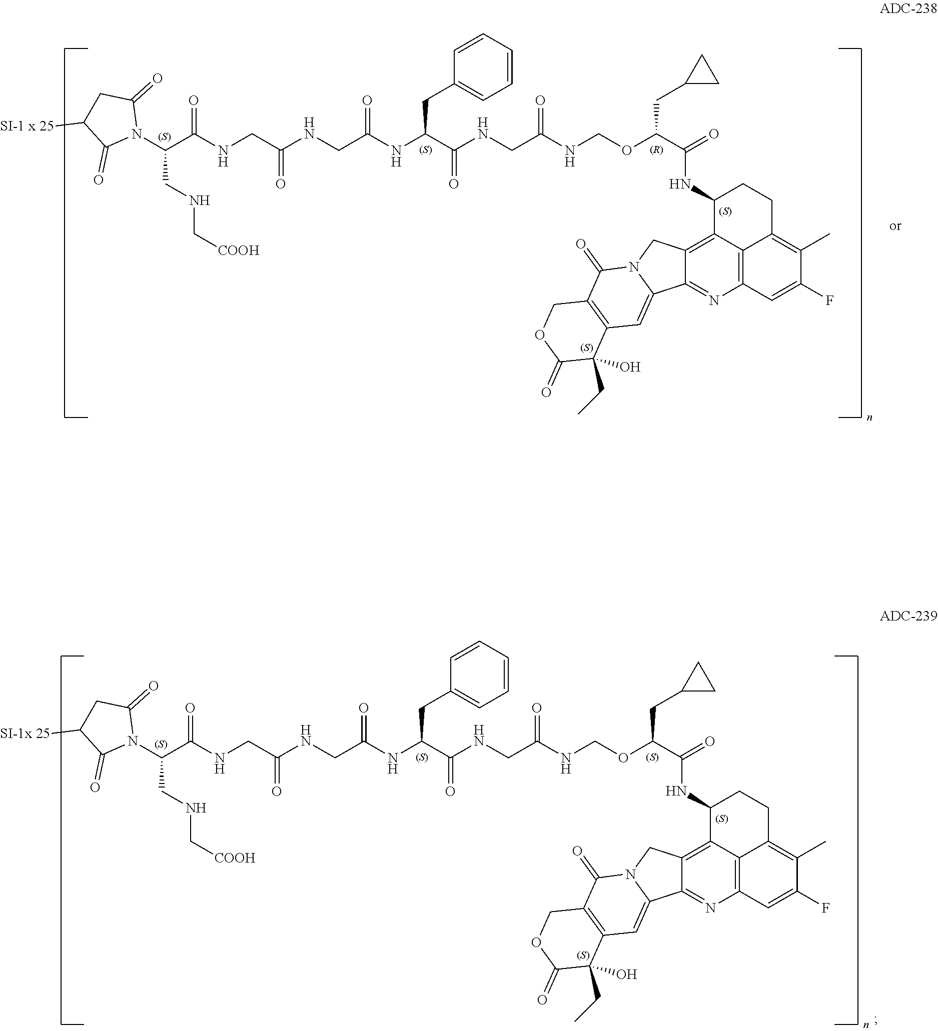

### Example 314

ADC-240 to ADC-247 were prepared according to the general-purpose coupling method of Example 94,

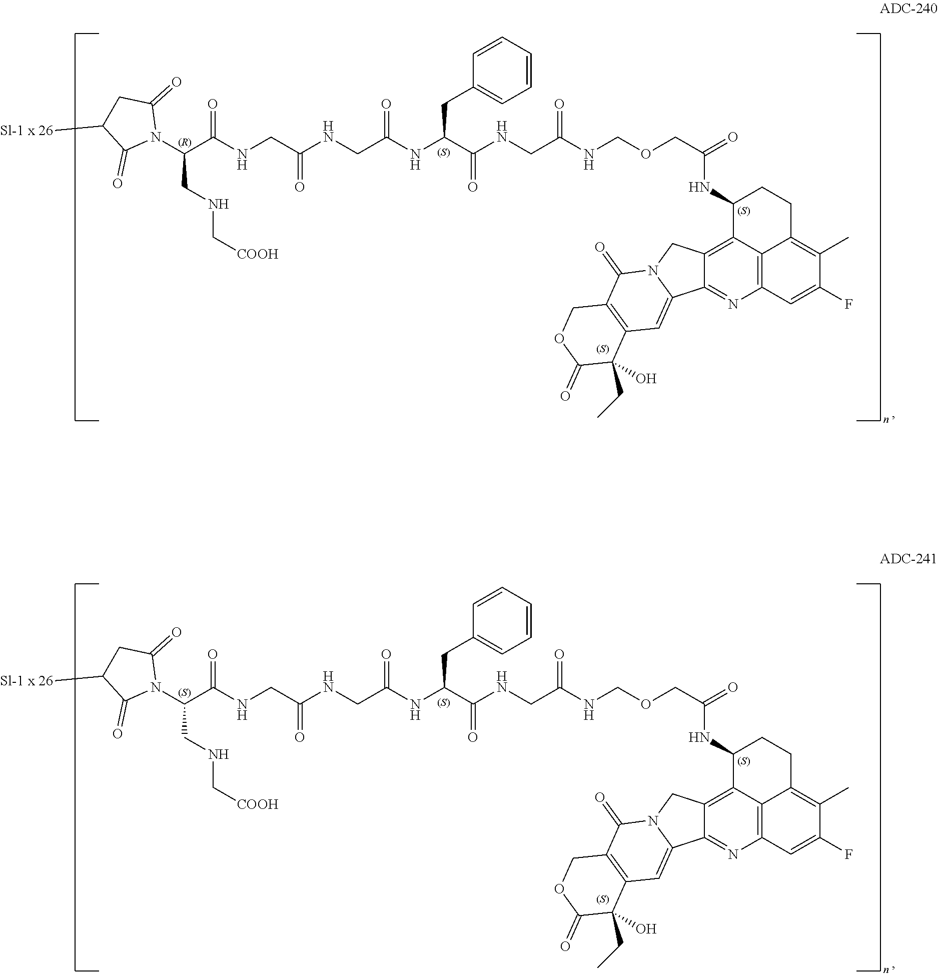

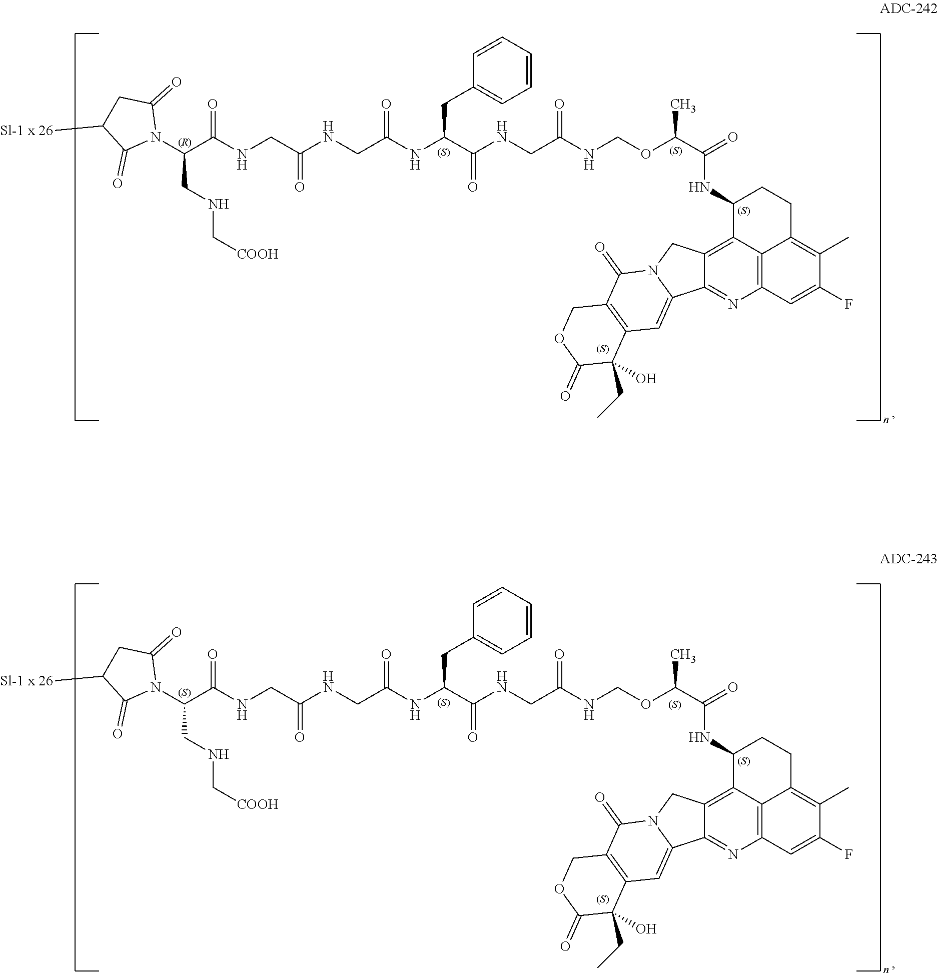

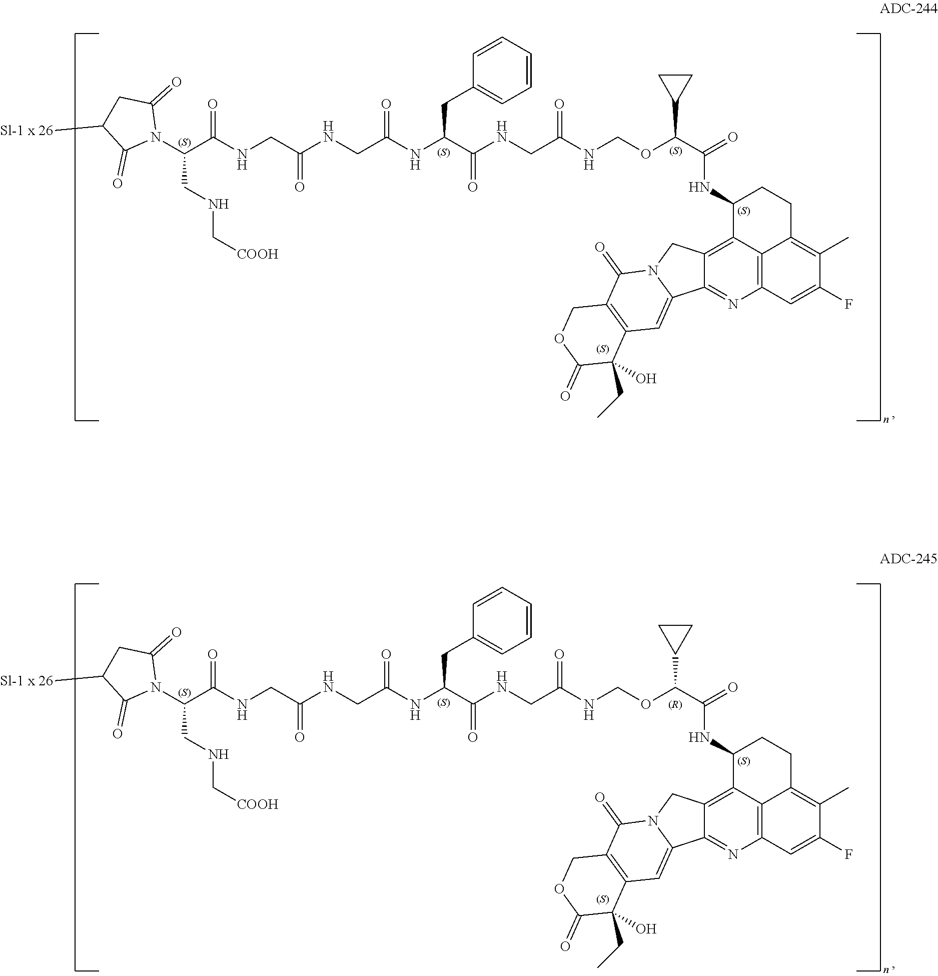

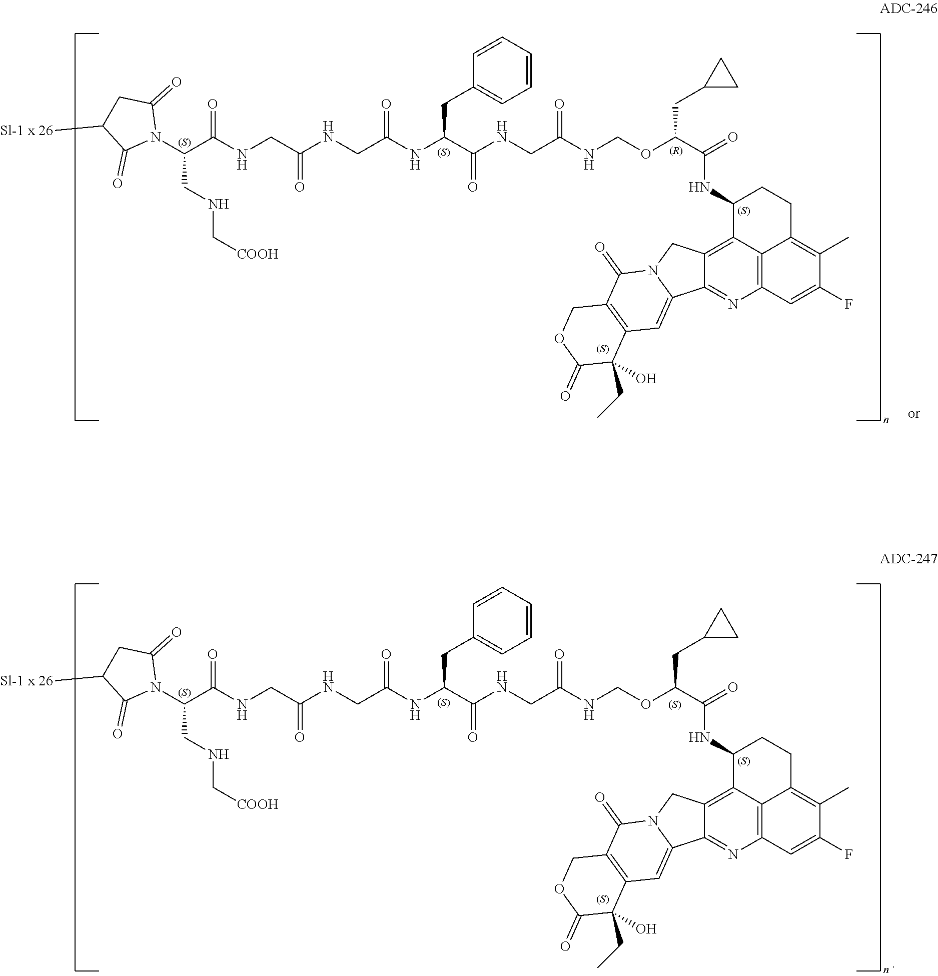

### Example 315

The monomer rate was determined using the SEC-HPLC method:
Chromatography column: Biocore SEC-300 5 m, 4.6×300 mm
Manufacturer: NanoChrom, Item No.: B213-050030-04630m
Mobile phase: 50 mM PB+300 mM NaCl+200 mM Arg+5% IPA, pH=6.5

TABLE 1
Methodological parameters

| Parameters | Settings |
|---|---|
| Flow rate | 0.3 mL/min |
| Wavelengths | 214 nm and 280 nm |
| Column temperature | 30° C. |
| Sample plate temperature | room temperature |
| Injection volume | 20 ug |
| Maximum pressure | 150 bar/15 MPa/2175 PSI |
| Gradient | isocratic |
| Operating time | 20 minutes |

TABLE 2
Monomer rate data for the disclosed ligand-drug conjugate
(ADC) disclosed in the present application

| | | | Monomer |
|---|---|---|---|
| Molecule name | Degradation % | Aggregate % | rate % |
| ADC-1 | 0.01 | 1.60 | 98.39 |
| ADC-2 | 0.0 | 1.51 | 98.49 |
| ADC-5 | 0.0 | 2.43 | 97.57 |
| ADC-6 | 0.0 | 2.42 | 97.58 |
| ADC-7 | 0.12 | 2.16 | 97.72 |
| ADC-8 | 0.07 | 1.37 | 98.56 |
| ADC-10 | 0.05 | 1.35 | 98.60 |
| ADC-11 | 0.11 | 1.51 | 98.38 |
| ADC-12 | 0.05 | 1.62 | 98.33 |
| ADC-13 | 0.08 | 1.30 | 98.62 |
| ADC-14 | 0.02 | 1.49 | 98.49 |
| ADC-48 | 0.06 | 1.71 | 98.23 |
| ADC-52 | 0.05 | 1.56 | 98.39 |
| ADC-DS | 0.0 | 2.82 | 97.18 |
| ADC-64 | 0.0 | 1.16 | 98.84 |
| ADC-68 | 0.01 | 0.44 | 98.55 |
| ADC-75 | 0.0 | 1.61 | 98.39 |
| ADC-81 | 0.2 | 1.59 | 98.39 |
| ADC-87 | 0.07 | 1.30 | 98.63 |
| ADC-96 | 0.08 | 1.32 | 98.60 |
| ADC-102 | 0.05 | 1.31 | 98.64 |
| ADC-108 | 0.0 | 6.36 | 93.64 |
| ADC-112 | 1.2 | 2.81 | 95.99 |
| ADC-113 | 0.03 | 1.22 | 98.75 |
| ADC-120 | 0.0 | 1.73 | 98.27 |
| ADC-122 | 0.01 | 1.44 | 98.55 |
| ADC-129 | 0.13 | 2.54 | 97.33 |
| ADC-130 | 0.37 | 2.18 | 97.45 |
| ADC-131 | 0.60 | 1.00 | 98.40 |
| ADC-132 | 0.03 | 2.22 | 97.75 |
| ADC-133 | 0.53 | 1.37 | 98.10 |
| ADC-145 | 0.11 | 1.24 | 98.65 |
| ADC-158 | 0.98 | 2.89 | 96.13 |
| ADC-160 | 0.10 | 1.45 | 98.45 |
| ADC-172 | 0.65 | 1.31 | 98.04 |
| ADC-179 | 0.1 | 1.06 | 98.84 |
| ADC-188 | 0.11 | 1.33 | 98.56 |
| ADC-193 | 0.11 | 1.51 | 98.38 |
| ADC-206 | 0.09 | 1.32 | 98.59 |
| ADC-214 | 0.03 | 1.18 | 98.79 |
| ADC-215 | 0.0 | 3.01 | 96.99 |
| ADC-219 | 0.0 | 7.35 | 92.65 |
| ADC-227 | 0.06 | 4.5 | 95.44 |
| ADC-235 | 0.1 | 6.83 | 93.07 |

CONCLUSION: The ADC disclosed in the present invention is characterized by a low degradation rate and a low aggregation rate, and has the excellent property of a high monomer rate.

### Example 316

The drug antibody ratio DAR was determined using the RP-HPLC method:
Chromatography column name: Proteomix RP-1000 4.6×100 mm 5 μm 1000A Manufacturer: Sepax

TABLE 3
Methodological parameters

| Parameters | Settings | | | |
|---|---|---|---|---|
| Mobile phase | A: 0.1% TFA aqueous solution; B: 0.1% TFA | | | |
| | acetonitrile solution | | | |
| Flow rate | 0.5 mL/min | | | |
| Wavelength | 214 nm and 280 nm | | | |
| Column | 65° C. | | | |
| temperature | | | | |
| Sample plate | room temperature | | | |
| temperature | | | | |
| Injection volume | 25 ug | | | |
| Maximum pressure | 100 bar/10 MPa/1450 PSI | | | |
| | Time | Flow rate | Mobile phase | Mobile phase |
| | (min) | (mL/min) | A (%) | B (%) |
| Gradient | 0.0 | 0.5 | 75 | 25 |
| | 3 | 0.5 | 75 | 25 |
| | 28 | 0.5 | 50 | 50 |
| | 30 | 0.5 | 5 | 95 |
| | 32 | 0.5 | 5 | 95 |
| | 33 | 0.5 | 75 | 25 |
| | 40 | 0.5 | 75 | 25 |

TABLE 4
Detailed data on ADC drug-antibody coupling ratio (DAR)

| Sample | Batch | | Sample | Batch | |
|---|---|---|---|---|---|
| Name | number | RP-DAR | Name | number | RP-DAR |
| ADC-5 | 20201028 | 7.65 | ADC-120 | 20200708 | 7.83 |
| ADC-6 | 20200923 | 6.99 | ADC-122 | 20200708 | 6.97 |
| ADC-10 | 20200104 | 7.38 | ADC-137 | 20191125 | 7.44 |
| ADC-12 | 20191125 | 7.61 | ADC-140 | 20200708 | 7.52 |
| ADC-18 | 20201028 | 7.71 | ADC-142 | 20201028 | 7.50 |
| ADC-23 | 20200104 | 7.19 | ADC-143 | 20201028 | 7.61 |
| ADC-24 | 20200104 | 7.09 | ADC-159 | 20200708 | 7.28 |
| ADC-36 | 20200708 | 7.44 | ADC-160 | 20200708 | 7.89 |
| ADC-41 | 20201028 | 7.81 | ADC-168 | 20201028 | 7.24 |
| ADC-42 | 20201028 | 7.55 | ADC-169 | 20201028 | 7.89 |
| ADC-55 | 20200708 | 7.54 | ADC-170 | 20201028 | 7.55 |
| ADC-59 | 20200708 | 7.57 | ADC-171 | 20201028 | 6.91 |
| ADC-64 | 20201028 | 7.42 | ADC-176 | 20200708 | 7.32 |
| ADC-70 | 20200104 | 7.60 | ADC-183 | 20200708 | 7.18 |
| ADC-71 | 20200104 | 7.50 | ADC-184 | 20200708 | 7.49 |
| ADC-72 | 20200104 | 7.42 | ADC-185 | 20200708 | 7.30 |
| ADC-88 | 20200708 | 7.31 | ADC-186 | 20200708 | 7.77 |
| ADC-96 | 20191125 | 7.75 | ADC-190 | 20200708 | 7.26 |
| ADC-100 | 20200708 | 7.56 | ADC-199 | 20191125 | 7.42 |
| ADC-108 | 20200708 | 7.62 | ADC-214 | 20201028 | 7.85 |
| ADC-112 | 20201116 | 7.46 | ADC-215 | 20211013 | 7.38 |
| ADC-219 | 20211015 | 7.14 | ADC-227 | 20211013 | 7.46 |
| ADC-235 | 20211013 | 7.26 | ADC-243 | 20211027 | 7.38 |

CONCLUSION: The ADC disclosed in the present invention has the excellent property of high DAR value, which can significantly increase the concentration of the drug at the target site location at the same dose of ADC drug administered.

### Example 317

ADC maintained the affinity of the corresponding original bispecific antibodies SI-1×6.4, SI-1×4, SI-1×22, SI-1×24, SI-1×25 and SI-1×26 for EGFR and HER3:

The relative affinities of SI-1×6.4 versus ADC-6 and SI-1×4 versus ADC-112 for EGFR and HER3 were compared by double-antigen sandwich ELISA. The specific steps were as follows:

Recombinant EGFR-His*6 antigen-coated plates were closed with 1% bovine serum protein; then SI-1×6.4, ADC-6, SI-1×4, and ADC-112 were diluted, respectively, and then diluted with a starting concentration of 5,000 ng/mL in successive 3-fold gradients, for a total of 11 concentrations; the samples were incubated on the coated ELISA plates for a certain period of time, followed by biotin-labeled HER3-Fc antigen incubation, followed by streptavidin HRP-labeled incubation; finally, TMB color development was performed, followed by sulfuric acid solution termination, and the absorbance value at 450 nm was detected on a microplate reader. The assay results were plotted against concentration for 0D450 nm.

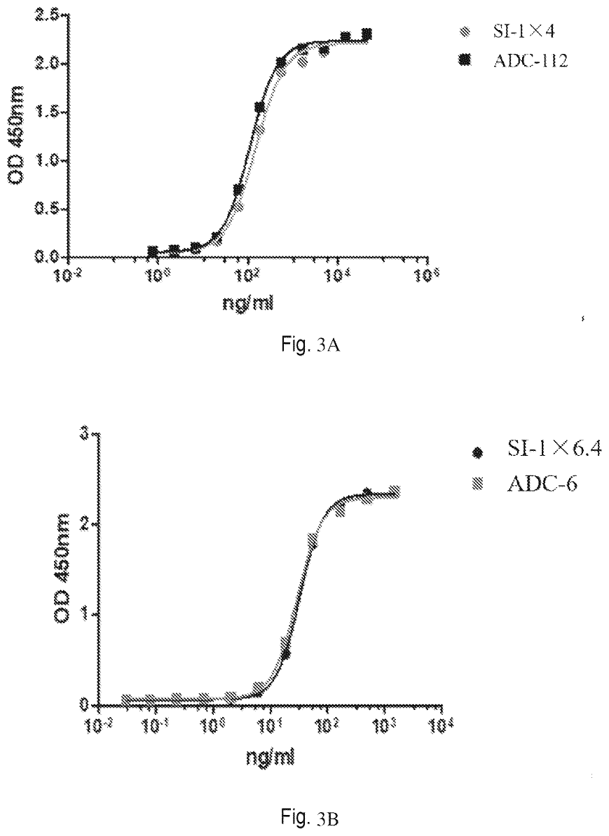

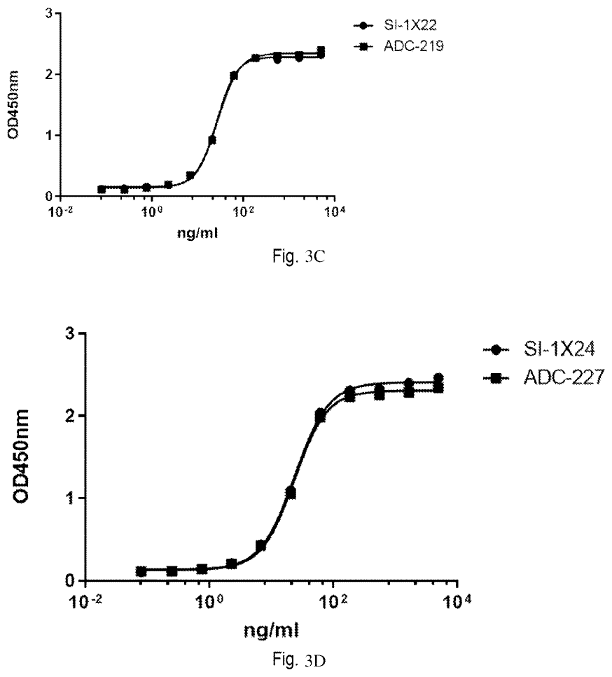

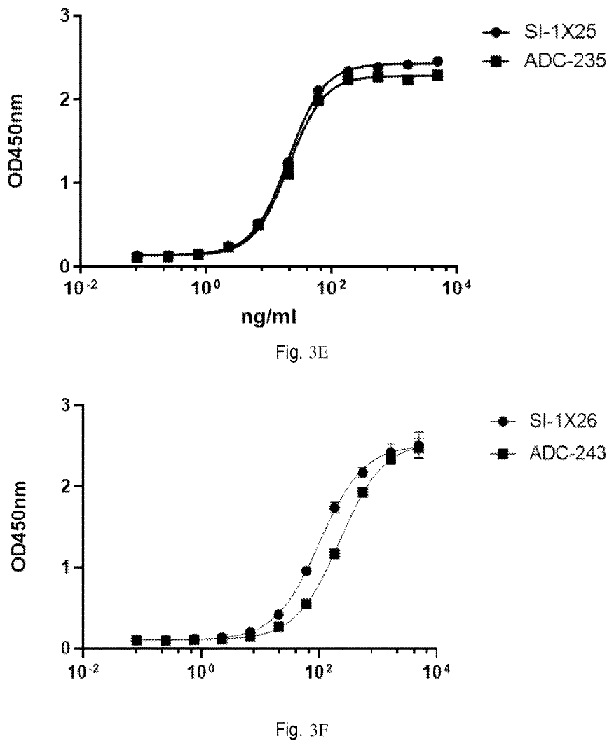

CONCLUSION: As shown in the accompanying FIGS. 3A and 3B, after coupling, ADC-6 and ADC-112 maintained similar affinities to those of SI-1×6.4 and SI-1×4, respectively, with no significant difference in EC50 values; indicating that coupling of toxins to SI-1×6.4 and SI-1×4 did not affect their affinity for the antigen.

Similarly, using a similar testing method to that described above, the results are shown in FIG. 3C, FIG. 3D, FIG. 3E, and FIG. 3F; after coupling, ADC-219, ADC-227, ADC-235, and ADC-243 retained similar affinities to those of SI-1×22, SI-1×24, SI-1×25, and SI-1×26, respectively, with no significant difference in the EC50 values; indicating that coupling of toxins to SI-1×22, SI-1×24, SI-1×25 and SI-1×26 did not affect their affinity for the antigen.

### Example 318

### In Vitro Pharmacodynamic Assays:

A variety of human-derived tumor cell lines (human epidermal cancer cells A431, human in situ pancreatic adenocarcinoma cells BXPC-3, human pharyngeal squamous cell carcinoma cells FaDu, human lung cancer squamous cell carcinoma cell line HARA-B, human non-small cell lung cancer cells HCC827, and human colon cancer cells SW620) were utilized as experimental models for evaluating in vitro efficacy of the ADC-coupled drug in the present invention. A certain number of tumor cells were inoculated in a 96-well plate, and the gradient-diluted test antibody and the corresponding ADC drug were added to the cells, followed by 5 days of treatment; the cell viability was measured using Alamar Blue or MTS, and the inhibitory effect of the test antibody and ADC on the tumor cell lines was evaluated by calculating the IC50. The starting concentration of the antibody drug was 500 nM, and the dilution was 7-fold, totaling 8 concentration points, and the treatment was carried out for 5 days. The final calculation method was survival rate=(experimental group−blank)/(control group−blank group)×10000, then Graph Pad Prism was used for curve fitting, to calculate the half inhibitory concentration (IC50) as well as Efficacy (0%).

TABLE 5
In vitro efficacy of six naked antibodies (SI-1 × 4, SI-1 × 6.4, SI-1 × 22,
SI-1 × 24, SI-1 × 25, SI-1 × 26) and six ADCs (ADC-112, ADC-6,
ADC-219, ADC-227, ADC-235, ADC-243) in A431
A431

| Groups | IC₅₀ (nM) | Groups | Efficacy (%) |
|---|---|---|---|
| ADC-112 | 4.95 | ADC-112 | 85.63 |
| ADC-6 | 0.98 | ADC-6 | 87.88 |
| ADC-219 | 0.75 | ADC-219 | 89.37 |
| ADC-227 | 0.77 | ADC-227 | 86.13 |
| ADC-235 | 0.81 | ADC-235 | 86.86 |
| ADC-243 | 3.61 | ADC-243 | 89.63 |
| SI-1 × 4 | >500 | SI-1 × 4 | 31.99 |
| SI-1 × 6.4 | 6.98 | SI-1 × 6.4 | 62.18 |
| SI-1 × 22 | 4.47 | SI-1 × 22 | 69.01 |
| SI-1 × 24 | 6.23 | SI-1 × 24 | 69.98 |
| SI-1 × 25 | 5.54 | SI-1 × 25 | 75.89 |
| SI-1 × 26 | >500 | SI-1 × 26 | 44.63 |

TABLE 6
In vitro efficacy of six naked antibodies (SI-1 × 4, SI-1 × 6.4, SI-1 × 22,
SI-1 × 24, SI-1 × 25, SI-1 × 26) and six ADCs (ADC-112, ADC-6,
ADC-219, ADC-227, ADC-235, ADC-243) in BXPC-3
BxPC-3

| Groups | IC₅₀ (nM) | Groups | Efficacy (%) |
|---|---|---|---|
| ADC-112 | 46.75 | ADC-112 | 61.96 |
| ADC-6 | 12.29 | ADC-6 | 61.02 |
| ADC-219 | 16.58 | ADC-219 | 65.64 |
| ADC-227 | 32.48 | ADC-227 | 68.85 |
| ADC-235 | 15.69 | ADC-235 | 68.24 |
| ADC-243 | 16.69 | ADC-243 | 65.96 |
| SI-1 × 4 | >500 | SI-1 × 4 | N/A |
| SI-1 × 6.4 | >500 | SI-1 × 6.4 | N/A |
| SI-1 × 22 | >500 | SI-1 × 22 | N/A |
| SI-1 × 24 | >500 | SI-1 × 24 | N/A |
| SI-1 × 25 | >500 | SI-1 × 25 | N/A |
| SI-1 × 26 | >500 | SI-1 × 26 | N/A |

TABLE 7
In vitro efficacy of six naked antibodies (SI-1 × 4, SI-1 × 6.4, SI-1 × 22,
SI-1 × 24, SI-1 × 25, SI-1 × 26) and six ADCs (ADC-112, ADC-6,
ADC-219, ADC-227, ADC-235, ADC-243) in FaDu
FaDu

| Groups | IC₅₀ (nM) | Groups | Efficacy (%) |
|---|---|---|---|
| ADC-112 | 1.01 | ADC-112 | 74.09 |
| ADC-6 | 0.18 | ADC-6 | 74.31 |
| ADC-219 | 0.21 | ADC-219 | 74.68 |
| ADC-227 | 0.25 | ADC-227 | 72.44 |
| ADC-235 | 0.28 | ADC-235 | 74.09 |
| ADC-243 | 0.40 | ADC-243 | 72.63 |
| SI-1 × 4 | >500 | SI-1 × 4 | 23.34 |
| SI-1 × 6.4 | >500 | SI-1 × 6.4 | 38.92 |
| SI-1 × 22 | >500 | SI-1 × 22 | 39.45 |
| SI-1 × 24 | >500 | SI-1 × 24 | 36.18 |
| SI-1 × 25 | >500 | SI-1 × 25 | 41.65 |
| SI-1 × 26 | >500 | SI-1 × 26 | 34.43 |

TABLE 8
In vitro efficacy of six naked antibodies (SI-1 × 4, SI-1 × 6.4, SI-1 × 22,
SI-1 × 24, SI-1 × 25, SI-1 × 26) as well as six ADCs (ADC-112,
ADC-6, ADC-219, ADC-227, ADC-235, ADC-243) in HARA-B.
HARA-B

| Groups | IC₅₀ (nM) | Groups | Efficacy (%) |
|---|---|---|---|
| ADC-112 | 14.73 | ADC-112 | 60.67 |
| ADC-6 | 3.89 | ADC-6 | 73.43 |
| ADC-219 | 2.37 | ADC-219 | 73.4 |
| ADC-227 | 2.45 | ADC-227 | 71.07 |
| ADC-235 | 3.18 | ADC-235 | 73 |
| ADC-243 | 9.93 | ADC-243 | 64.77 |
| SI-1 × 6.4 | >500 | SI-1 × 6.4 | 39.27 |
| SI-1 × 22 | >500 | SI-1 × 22 | 43.36 |
| SI-1 × 24 | >500 | SI-1 × 24 | 43.65 |
| SI-1 × 25 | >500 | SI-1 × 25 | 44.73 |
| SI-1 × 26 | >500 | SI-1 × 26 | 36.55 |
| SI-1 × 4 | >500 | SI-1 × 4 | 44.32 |

TABLE 9
In vitro efficacy of six naked antibodies (SI-1 × 4, SI-1 × 6.4, SI-1 × 22,
SI-1 × 24, SI-1 × 25, SI-1 × 26) and six ADCs (ADC-112,
ADC-6, ADC-219, ADC-227, ADC-235, ADC-243) in HCC827.
HCC827

| Groups | IC₅₀ (nM) | Groups | Efficacy (%) |
|---|---|---|---|
| ADC-112 | 2.35 | ADC-112 | 73.10 |
| ADC-6 | 1.06 | ADC-6 | 71.31 |
| ADC-219 | 0.65 | ADC-219 | 71.21 |
| ADC-227 | 0.51 | ADC-227 | 66.61 |
| ADC-235 | 0.43 | ADC-235 | 68.22 |
| ADC-243 | 2.11 | ADC-243 | 69.56 |
| SI-1 × 4 | >500 | SI-1 × 4 | 18.52 |
| SI-1 × 6.4 | 2.08 | SI-1 × 6.4 | 60.02 |
| SI-1 × 22 | 1.42 | SI-1 × 22 | 63.69 |
| SI-1 × 24 | 1.48 | SI-1 × 24 | 66.70 |
| SI-1 × 25 | 1.32 | SI-1 × 25 | 69.43 |
| SI-1 × 26 | >500 | SI-1 × 26 | 28.44 |

TABLE 10
In vitro efficacy of six naked antibodies (SI-1 × 4, SI-1 × 6.4, SI-1 × 22,
SI-1 × 24, SI-1 × 25, SI-1 × 26) and six ADCs (ADC-112, ADC-6,
ADC-219, ADC-227, ADC-235, ADC-243) in SW620.
SW620

| Groups | IC₅₀ (nM) | Groups | Efficacy (%) |
|---|---|---|---|
| ADC-112 | 5.82 | ADC-112 | 70.72 |
| ADC-6 | 4.51 | ADC-6 | 75.35 |
| ADC-219 | 4.20 | ADC-219 | 75.56 |
| ADC-227 | 5.78 | ADC-227 | 70.76 |
| ADC-235 | 4.69 | ADC-235 | 73.94 |
| ADC-243 | 3.91 | ADC-243 | 69.68 |
| SI-1 × 4 | >500 | SI-1 × 4 | 4.02 |
| SI-1 × 6.4 | >500 | SI-1 × 6.4 | 0.24 |
| SI-1 × 22 | >500 | SI-1 × 22 | 3.45 |
| SI-1 × 24 | >500 | SI-1 × 24 | 5.61 |
| SI-1 × 25 | >500 | SI-1 × 25 | 5.92 |
| SI-1 × 26 | >500 | SI-1 × 26 | 6.47 |

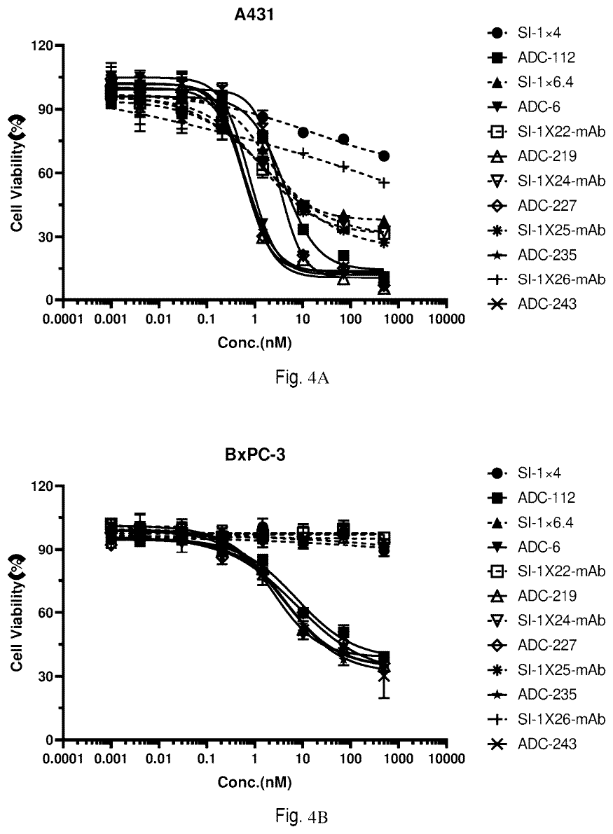

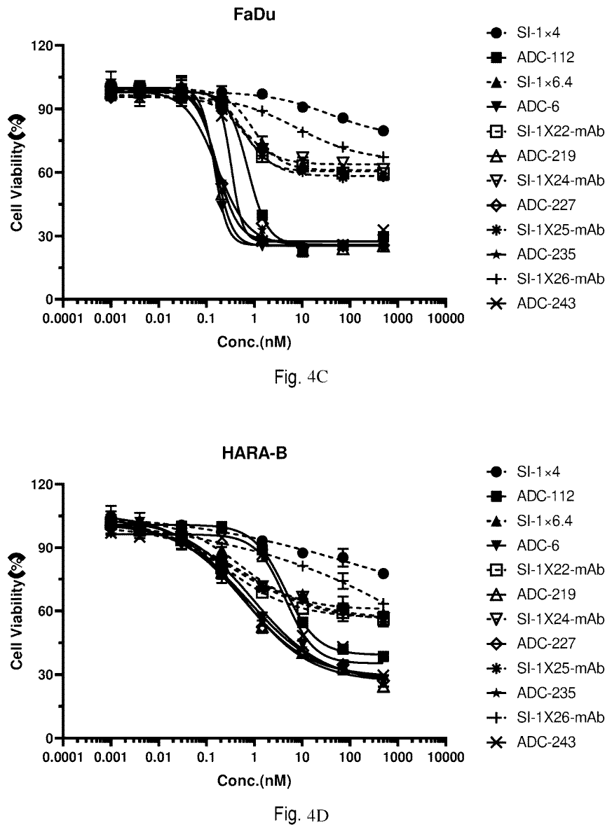

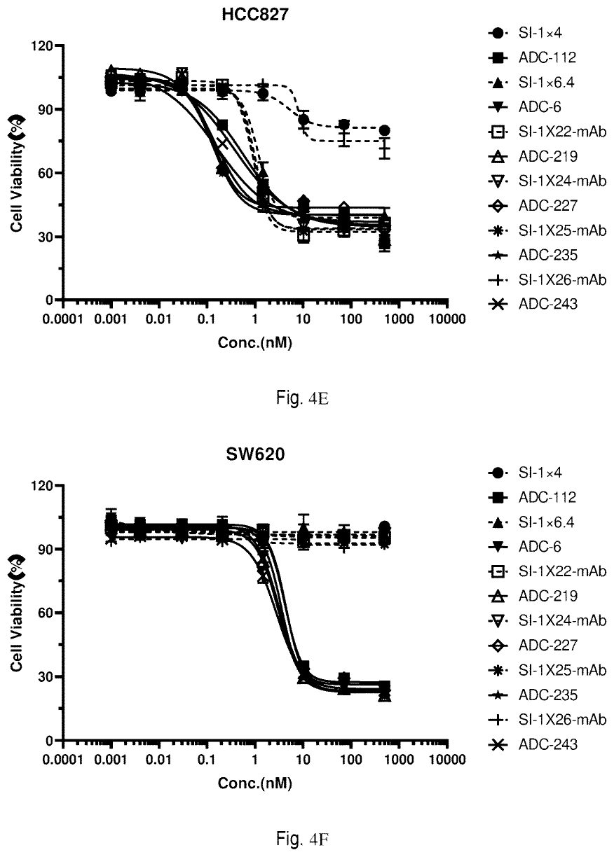

CONCLUSION: In human epidermal cancer cells A431, human in situ pancreatic adenocarcinoma cells BXPC-3, human pharyngeal squamous cell carcinoma cells FaDu, human lung cancer squamous cell carcinoma cell line HARA-B, human non-small-cell lung cancer cells HCC827, and human colon cancer cells SW620, six ADCs (ADC-112, ADC-6, ADC-219, ADC-227, ADC-235, ADC-243) all showed more sensitive efficacy and stronger tumor cell growth inhibition relative to their respective corresponding naked antibodies (SI-1×4, SI-1×6.4, SI-1×22, SI-1×24, SI-1×25, SI-1×26) (FIG. 4A-FIG. 4F and Table 5-Table 10).

### Example 319

### In Vivo Efficacy Testing:

In the present invention, BALB/c nude mice were subcutaneously inoculated with a variety of human tumor cell lines (A431, SW620, A431+SW620) as experimental models to evaluate the in vivo efficacy of ADC-coupled drugs. A certain number of tumor cell lines were inoculated subcutaneously in BALB/c nude mice, and when the tumor volume grew to 150-300 mm³, the antibodies and corresponding ADC drugs were injected into the tail vein; the drugs were administered once a week four times, with continuous observation, and tumor measurements were performed twice a week to evaluate the inhibitory effect of the test antibodies and ADCs on the tumor cell lines.

### Conclusion:

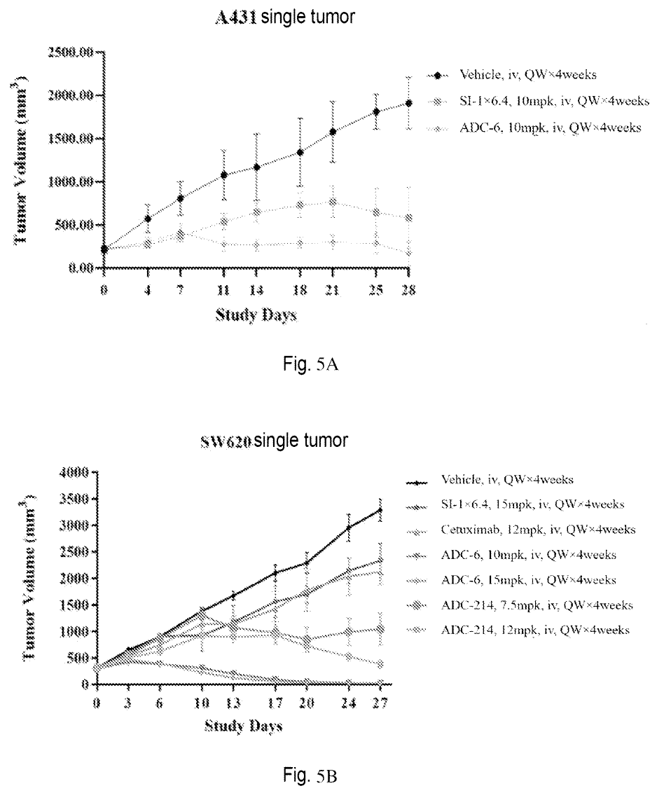

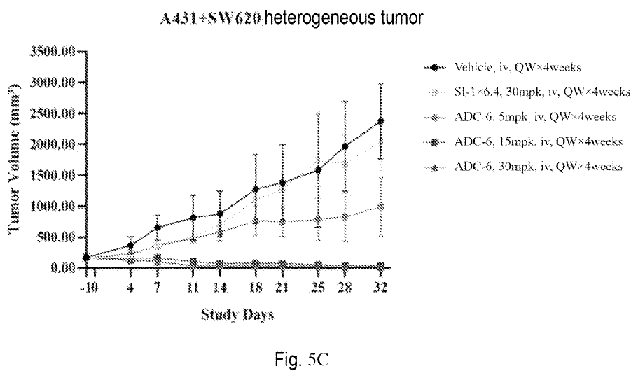

In the A431 single-tumor model with high expression of EGFR, 10 mg/kg of ADC-6 (DAR=8) exhibited stronger tumor inhibition relative to SI-1×6.4 naked antibody (FIG. 5A).

In the SW620 single-tumor model with low EGFR expression, 10 and 15 mg/kg of ADC-6 also exhibited stronger tumor suppression relative to 15 mg/kg of SI-1×6.4 naked antibody; and was stronger than the tumor suppression of Cet-ADC (ADC-214, DAR=8) at a dose corresponding to the molecular molar concentration (FIG. 5B).

In A431+SW620 heterogeneous tumors, 5, 15, and 30 mg/kg of ADC-6 similarly demonstrated stronger tumor inhibition relative to 30 mg/kg of SI-1×6.4 naked antibody (FIG. 5C).

### Example 320

### In Vivo Efficacy Testing:

BALB/c Nude mice subcutaneously inoculated with human epidermal cancer cells A431 and human pancreatic cancer cells BxPC3 were utilized as experimental models in the present invention to evaluate the in vivo efficacy of ADC-coupled drugs. A certain number of tumor cell lines were inoculated subcutaneously on the right shoulder side of female BALB/c Nude mice, and when the average tumor volume grew to 180-250 mm³, the corresponding ADC drugs were injected into the tail vein once a week for four consecutive administrations under continuous observation, and the tumor volume was measured twice a week to evaluate the inhibitory effect of the test ADC drugs on the growth of the tumors.

$$\% change to D 0 = ( Dn - D 0 ) / D 0 * 100 ;$$

$$\% TGI = 1 - [ changes of tumor volume in treatment group / changes of tumor volume in control group ] \times 100.$$

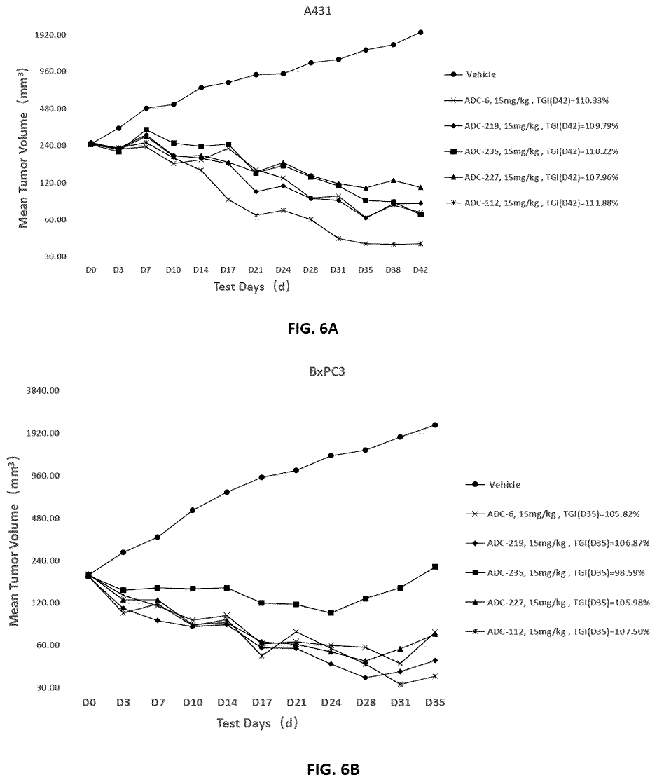

In the EGFR high-expressing A431 single-body tumor model, 42 days after the first administration, each test ADC drug caused significant tumor growth inhibition (P<0.05) in the BALB/c-Nude mouse subcutaneous graft tumor model of human epidermal cancer cells A431, wherein ADC-6, ADC-219, ADC-235, ADC-227 and ADC-112 had comparable tumor-suppressing effects, all of them showing strong tumor-suppressing effects (FIG. 6A).

In the BxPC3 single-body tumor model with medium expression of EGFR, 35 days after the first administration, each test ADC drug caused significant tumor growth inhibition (P<0.05) in the human epidermal cancer cell BxPC3 BALB/c-Nude mouse subcutaneous graft tumor model, wherein ADC-6, ADC-219, ADC-235, ADC-227 and ADC-112 had comparable tumor-suppressing effects, all showing strong tumor-suppressing effects (FIG. 6B).

### Example 321

### In Vitro Efficacy of Payload Compound 5A:

### 1) Experimental Materials:

Cells: Cells for testing were obtained from the Cell Bank of the Chinese Academy of Sciences;
Cell culture medium DMEM: Gibco;
FBS: BIOWEST.

### 2) Preparation of Culture Medium:

Growth medium (with 10% FBS, Penicillin/streptomycin (100 U/mL);
Detection medium (with 1% FBS, Penicillin/streptomycin (100 U/mL).

### 3) Operation:

Turn on the UV light of the biosafety cabinet 30 min in advance, and then turn on the ventilation for 3 min. Put the growth medium, detection medium, D-PBS and pancreatin into a 37° C. constant-temperature water bath to preheat, and then sterilize the surface with alcohol, and put into the biosafety cabinet. Place cells with about 80% confluence in the biosafety cabinet, draw off the old medium, rinse with D-PBS, draw and discard, digest with pancreatin for 2-3 min, and then the growth medium for neutralization, and centrifuge at 1200 rpm for 3 min. Draw off the centrifugation supernatant, mix evenly with 4 mL of detection medium, and collect 100 uL for counting (wherein 50 uL of cell fluid is taken out, 50 uL of Trypan Blue Stain is added and mixed evenly, followed by counting). Plate spreading was performed according to a pre-optimized cell spreading density; 80 ul/well was spread in a 96-well plate, with only 80 uL of detection medium being added to wells E11 and F11, and 150 uL of DPBS being added to the edge wells. 24 h after spreading the plate, diluted antibody was added, 20 uL per well, and a control was set up; only 20 uL of detection medium was added to the 11th column, and 2 duplicate wells were set up for each concentration; after addition, uniform mixing was performed on a cell vortex mixer, at 550 rpm for 3 min.

Dilution of solution: Use detection medium to prepare 300 μL of a test sample solution with a starting concentration of 5 uM in the first column of a V-shaped 96-well plate, add 240 μL of detection medium to the second to 10th columns respectively, take 60 uL from the evenly mixed first column and add it to the second column, mix uniformly up and down with a multi-channel pipette 10 times, discard the pipette tips, and perform operations for the next 7 concentrations in turn.

### 4) Detection:

After 4 days, take out the MTS reagent, thaw at room temperature in the dark, then mix thoroughly and evenly using a vortex mixer, then add 20 μL of CellTiter One Solution Reagen MTS reagent per 100 L of cell culture volume along the side wall of the wells in the biosafety cabinet, gently tap the plate surface to mix the MTS solution evenly, and then put into a cell culture incubator and leave to incubate for 2 h in the dark. At the end of the reaction, take out the 96-well plate and measure the OD490 nm absorbance value in the microplate reader, and record, organize, analyze and store the data.

### 5) Results:

Table 11 illustrates that compound 5A (Payload) showed good inhibition of the following solid tumor cells and hematoma cells.

TABLE 11
In vitro inhibitory effect of compound 5A on human non-small
cell lung adenocarcinoma cells H1975, human non-small cell lung
cancer cells HCC827, human epidermal cancer cells A431, human
gastric cancer cells NCI-N87, human in situ pancreatic adenocarcinoma
cells BXPC-3, human epidermal cancer cells A431 + human
colon cancer cells SW620, human breast cancer cells ZR-75-1,
human plasma cell leukemia cells H929, human multiple myeloma
cells RPMI8226, human leukemia cells JJN-3, human breast cancer
cells MDA-MB-361 and human breast cancer cells SK-BR-3.

| Group | IC50 (nM) | Efficacy % |
|---|---|---|
| H1975 | 970.33 | 76.78 |
| HCC827 | 655.60 | 80.57 |
| A431 | 438.77 | 84.97 |
| NCI-N87 | 1023.92 | 63.93 |
| BxPC-3 | 320.09 | 64.4 |
| A431 + SW620 | 604.87 | 68.26 |
| ZR-75-1 | 557.95 | 54.79 |
| H929 | 23.94 | 99.03 |
| RPMI8226 | 147.00 | 93.61 |
| JJN-3 | 36.11 | 80.59 |
| MDA-MB-361 | 572.13 | 62.34 |
| SK-BR-3 | 732.43 | 70.9 |

### Example 322: In Vitro Efficacy Data for ADC-6 and ADC-214

The in vitro efficacy of ADC-coupled drugs was evaluated using an experimental model of two human tumor cell lines (human poorly differentiated lung cancer squamous cell carcinoma cell line Oka-c-1, human lung squamous cell carcinoma cells SK-MES-1).

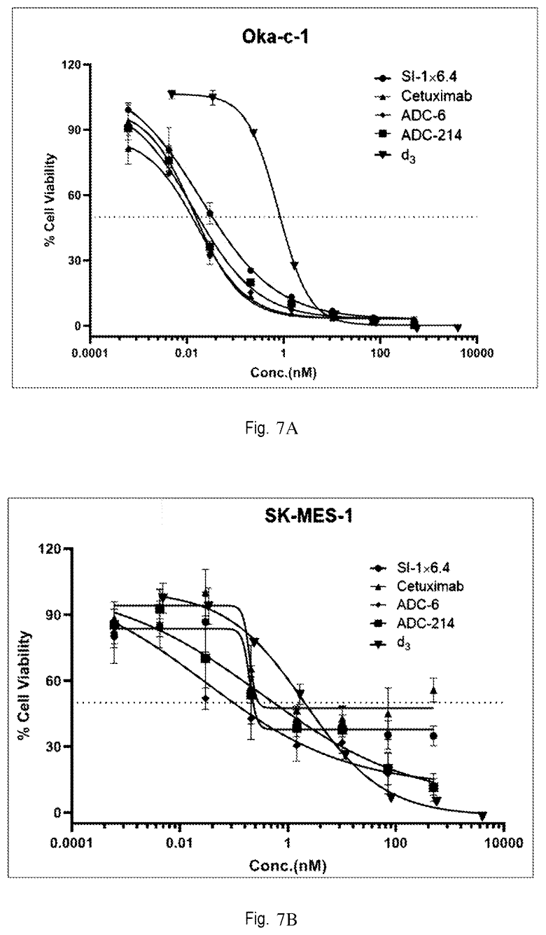

Inoculate a certain number of tumor cells in a 96-well plate, add the gradient-diluted test antibody and the corresponding ADC drug to the cells, treat for 5 days, measure the cell viability using Alamar Blue or MTS, and evaluate the inhibitory effect of the test antibody, the ADC and the small molecule drug d3 on the tumor cell lines by calculating the IC50. The starting concentration of the antibody drug was 500 nM, and the dilution was 7-fold, totaling 7 concentration points, and the treatment was carried out for 5 days. The final calculation method was survival rate=(experimental group−blank)/(control group−blank group)×100%, followed by curve fitting using Graph Pad Prism, and calculation of the half inhibitory concentration (IC50). The results are shown in FIGS. 7A and 7B and Table 12.

TABLE 12
In vitro efficacy of SI-1 × 6.4, Cetuximab, ADC-6,
ADC-214 and d3 on human poorly differentiated lung
cancer squamous cell carcinoma cell line Oka-c-1 and
human lung squamous cell carcinoma cells SK-MES-1.

| | IC50 (nM) | | | | |
|---|---|---|---|---|---|
| Group | SI-1 × 6.4 | Cetuximab | ADC-6 | ADC-214 | d₃ |
| Oka-c-1 | 0.033 | 0.015 | 0.013 | 0.017 | 0.846 |
| SK-MES-1 | 0.227 | 0.289 | 0.097 | 0.542 | 2.044 |

1.-49. (canceled)

50. An antibody-drug conjugate of formula I: 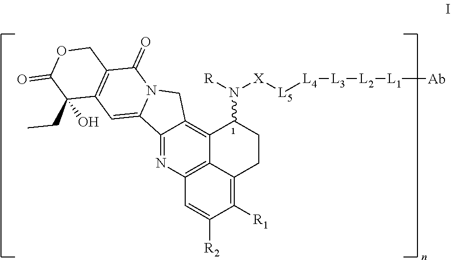
or a pharmaceutically acceptable salt or solvate thereof,
wherein:
Ab is a bispecific antibody or antigen-binding fragment thereof which simultaneously targets two different epitopes or targets;
L₁ is 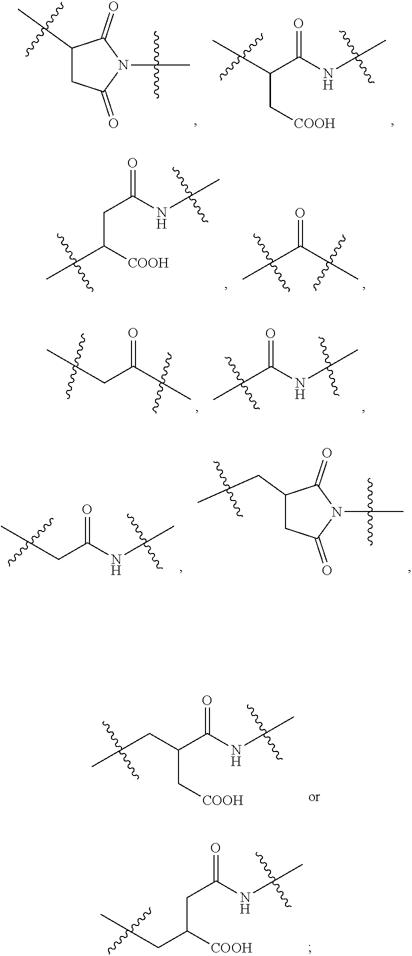
L₂ has the structure shown in formula A below: 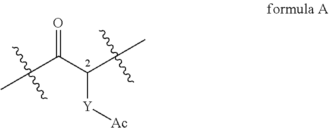
wherein Y is a scaffold selected from C1-C6 alkyl, substituted C1-C6 alkyl, or C3-C8 cycloalkyl; Ac is a hydrophilic structural unit; and the carbon No. 2 attached to Y has absolute chirality in the R configuration or the S configuration;
L₃ is present or absent, and when present, L₃ is 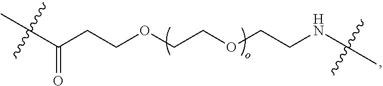
o is 1, 2, 3, 4, 5, 6, 7, 8, 9, or 10;
L₄ is an enzymatic cutting unit;
L₅ is a linking unit;
the chiral carbon atom No. 1 attached to N in formula I has absolute chirality in the R configuration or the S configuration;
R is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, C6-C10 aryl, substituted C6-C10 aryl, 5-10-membered heteroaryl, and substituted 5-10-membered heteroaryl;
R₁ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7 membered heterocyclyl, substituted 3-7 membered heterocyclyl, C6-C10 aryl, substituted C6-C10 aryl, 5-10 membered heteroaryl, and substituted 5-10 membered heteroaryl;
R₂ is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, carboxyl, 3-7 membered heterocyclyl, substituted 3-7 membered heterocyclyl, C6-C10 aryl, substituted C6-C10 aryl, 5-10 membered heteroaryl, and substituted 5-10 membered heteroaryl;
X is selected from —C(O)—CRₐR_{b}—(CR₃R₄)ₘ—O—, —C(O)—CRₐR_{b}—(CR₃R₄)ₘ—NH— or —C(O)—CRₐR_{b}—(CR₃R₄)ₘ—S—;
Rₐ and R_{b} are each independently selected from a hydrogen atom, a deuterium atom, a halogen, a C1-C6 alkyl, a deuterated C1-C6 alkyl, a halogenated C1-C6 alkyl, a C3-C8 cycloalkyl, a C3-C8 cycloalkyl C1-C6 alkyl, a C6-C10 aryl C1-C6 alkyl, a C1-C6 alkoxy C1-C6 alkyl, a 3-7 membered heterocyclic group, a substituted 3-7 membered heterocyclic group, a C6-C10 aryl, substituted C6-C10 aryl, 5-10 membered heteroaryl, and substituted 5-10 membered heteroaryl;
alternatively, Rₐ, R_{b} and the carbon atoms to which they are connected form a C3-C8 cycloalkyl group, a C3-C8 cycloalkyl C1-C6 alkyl group, a 3-7-membered heterocyclic group, or a substituted 3-7-membered heterocyclic group;
R₃, R₄ are identical or different and are each independently a hydrogen atom, deuterium atom, halogen, C1-C6 alkyl, halogenated C1-C6 alkyl, deuterated C1-C6 alkyl, C1-C6 alkoxy, hydroxyl, amino, cyano, nitro, hydroxy C1-C6 alkyl, C3-C8 cycloalkyl, 3-7-membered heterocyclyl, or substituted 3-7-membered heterocyclyl;
alternatively, R₃, R₄ and the carbon atoms connected thereto form a C3-C8 cycloalkyl group, a C3-C8 cycloalkyl C1-C6 alkyl group, a 3-7-membered heterocyclic group, or a substituted 3-7-membered heterocyclic group;
m is 0, 1, 2, 3, or 4; and
nis 1,2,3,4,5,6,7,8,9, or 10.

51. A linker-drug compound of formula III: 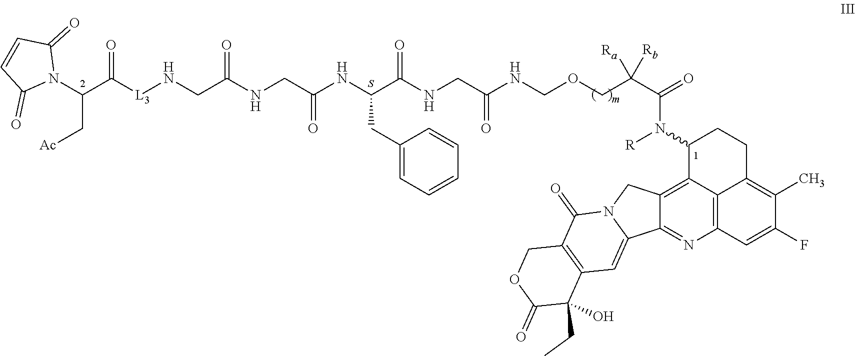
or a pharmaceutically acceptable salt or solvate thereof,
wherein:
R is selected from hydrogen atom, deuterium atom, halogen, C1-C6 alkyl, substituted C1-C6 alkyl, deuterated C1-C6 alkyl, C3-C8 cycloalkyl C1-C6 alkyl, C1-C6 alkoxy C1-C6 alkyl, C6-C10 aryl, substituted C6-C10 aryl, 5-10-membered heteroaryl, and substituted 5-10-membered heteroaryl;
Rₐ is selected from hydrogen atom, deuterium atom, halogen, C1-C6 alkyl, deuterated C1-C6 alkyl, halogenated C1-C6 alkyl, C3-C8 cycloalkyl, C3-C8 cycloalkyl C1-C6 alkyl, C1-C6 alkoxy C1-C6 alkyl, 3-7 membered heterocyclic group, substituted 3-7 membered heterocyclic group, C6-C10 aryl, substituted C6-C10 aryl, 5-10 membered heteroaryl, and substituted 5-10-membered heteroaryl;
R_{b} is selected from hydrogen atom, deuterium atom, halogen, C1-C6 alkyl, deuterated C1-C6 alkyl, halogenated C1-C6 alkyl, C3-C8 cycloalkyl, C3-C8 cycloalkyl C1-C6 alkyl, C1-C6 alkoxy C1-C6 alkyl, 3-7 membered heterocyclic group, substituted 3-7 membered heterocyclic group, C6-C10 aryl, substituted C6-C10 aryl, 5-10 membered heteroaryl, and substituted 5-10-membered heteroaryl;
alternatively, Rₐ, R_{b} and the carbon atoms attached thereto form a C3-C8 cycloalkyl group, a C3-C8 cycloalkyl C1-C6 alkyl group, a 3-7-membered heterocyclic group, or a substituted 3-7-membered heterocyclic group;
L₃ is present or absent, and when L₃ is present, it is 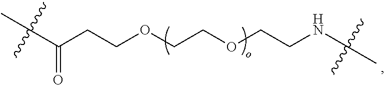
o is 1, 2, 3, 4, 5, 6, 7, 8, 9, or 10;
the 1-position or 2-position chiral carbon atom has two chiral configurations: the R absolute configuration or S absolute configuration;
Ac is a hydrophilic structural unit; and
m is 0 or 1.

52. A method for treating or preventing cancer, comprising administering to a subject in need thereof a therapeutically or prophylactically effective amount of the antibody-drug conjugate of claim 50.

53. A method for treating or preventing cancer, comprising administering to a subject in need thereof a therapeutically or prophylactically effective amount of the linker-drug compound of claim 51.

54. The method of claim 52, wherein the antibody-drug conjugate is: 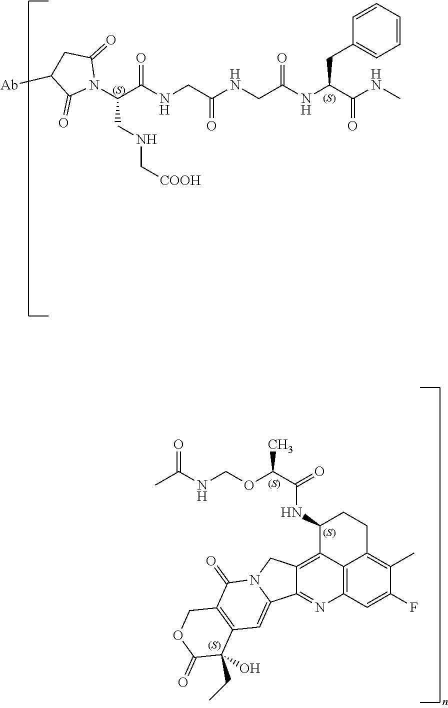
or a pharmaceutically acceptable salt thereof, wherein each instance of 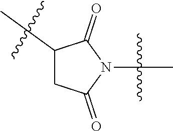 [SMILES: *C1CC(=O)N(*)C1=O]
is independently and optionally replaced by 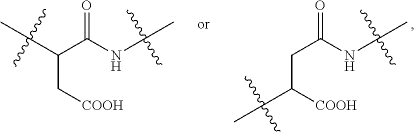
wherein Ab is a bispecific antibody or antigen-binding fragment thereof, wherein the Ab comprises an IgG having binding specificity for EGFR and a single-chain Fv (scFv) having binding specificity for HER3, wherein the IgG comprises a heavy chain and a κ light chain, wherein the heavy chain of the IgG comprises a heavy chain CDR1 comprising the amino acid sequence of SEQ ID NO:29, a heavy chain CDR2 comprising the amino acid sequence of SEQ ID NO:30, and a heavy chain CDR3 comprising the amino acid sequence of SEQ ID NO:31, and wherein the κ light chain of the IgG comprises a light chain CDR1 comprising the amino acid sequence of SEQ ID NO:25, a light chain CDR2 comprising the amino acid sequence of SEQ ID NO:26, and a light chain CDR3 comprising the amino acid sequence of SEQ ID NO:27; and
wherein the scFv comprises a heavy chain CDR1 comprising the amino acid sequence of SEQ ID NO:32, a heavy chain CDR2 comprising the amino acid sequence of SEQ ID NO:33, a heavy chain CDR3 comprising the amino acid sequence of SEQ ID NO:34, a light chain CDR1 comprising the amino acid sequence of SEQ ID NO:35, a light chain CDR2 comprising the amino acid sequence of SEQ ID NO:36, and a light chain CDR3 comprising the amino acid sequence of SEQ ID NO:37, and
wherein n is 1, 2, 3, 4, 5, 6, 7, 8, 9, or 10.

55. The method of claim 54, wherein the heavy chain of the IgG comprises a heavy chain variable region comprising the amino acid sequence of SEQ ID NO:38, and wherein the κ light chain of the IgG comprises a light chain variable region comprising the amino acid sequence of SEQ ID NO:28; and
wherein the scFv comprises a heavy chain variable region comprising the amino acid sequence of SEQ ID NO:39; and a light chain variable region comprising the amino acid sequence of SEQ ID NO:40.

56. The method of claim 52, wherein the antibody-drug conjugate is: 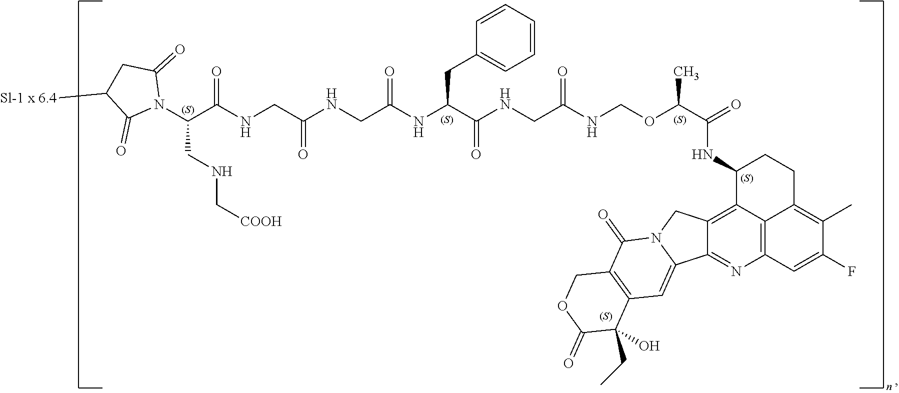
or a pharmaceutically acceptable salt thereof, wherein each instance of 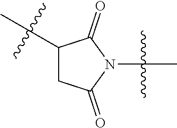 [SMILES: *C1CC(=O)N(*)C1=O]
is independently and optionally replaced by 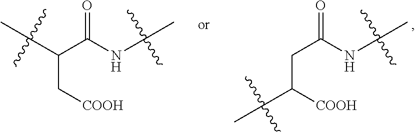 [SMILES: *NC(=O)C(*)CC(=O)O ; *NC(=O)CC(*)C(=O)O]
wherein SI-1×6.4 is a bispecific antibody comprising two IgG1 heavy chains, two κ light chains, and two scFvs, wherein the first IgG1 heavy chain and the first scFv form a construct comprising the amino acid sequence of SEQ ID NO: 4; the second IgG1 heavy chain and the second scFv form a construct comprising the amino acid sequence of SEQ ID NO: 4; the first κ light chain comprises the amino acid sequence of SEQ ID NO: 2; and the second κ light chain comprises the amino acid sequence of SEQ ID NO: 2, and
wherein n is 1, 2, 3, 4, 5, 6, 7, 8, 9, or 10.

57. The method of claim 56, wherein n is 6, 7, or 8.

58. The method of claim 56, wherein n is 8.

59. The method of claim 56, wherein the moiety of 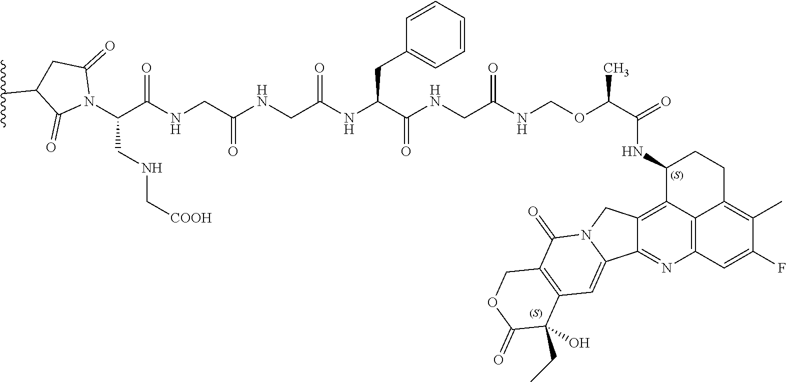
is covalently attached to one or more sulfur atoms of one or more cysteine residues of SI-1×6.4, and wherein each instance of 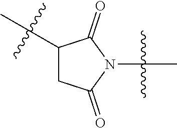 [SMILES: *C1CC(=O)N(*)C1=O]
is independently and optionally replaced by

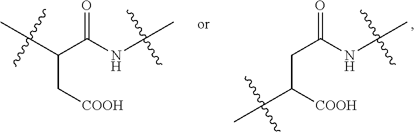 [SMILES: *NC(=O)C(*)CC(=O)O ; *NC(=O)CC(*)C(=O)O]

60. The method of claim 59, wherein the one or more cysteine residues are derived from the opening of one or more interchain disulfide bonds of SI-1×6.4.

61. The method of claim 52, wherein the antibody-drug-conjugate is: 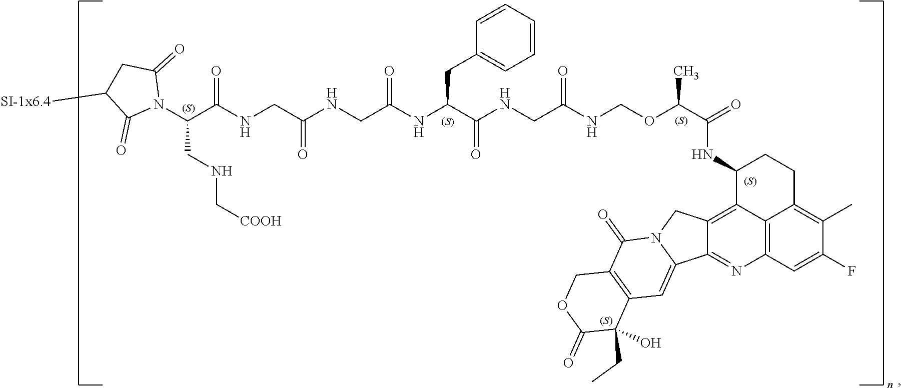
wherein each instance of 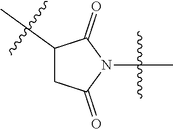 [SMILES: *C1CC(=O)N(*)C1=O]
is independently and optionally replaced by 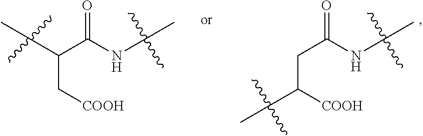 [SMILES: *NC(=O)C(*)CC(=O)O ; *NC(=O)CC(*)C(=O)O]
wherein SI-1×6.4 is a bispecific antibody comprising two IgG1 heavy chains, two κ light chains, and two scFvs, wherein the first IgG1 heavy chain and the first scFv form a construct comprising the amino acid sequence of SEQ ID NO: 4; the second IgG1 heavy chain and the second scFv form a construct comprising the amino acid sequence of SEQ ID NO: 4; the first κ light chain comprises the amino acid sequence of SEQ ID NO: 2; and the second κ light chain comprises the amino acid sequence of SEQ ID NO: 2, and
wherein n is 5, 6, 7, or 8.

62. The method of claim 61, wherein n is 6, 7, or 8.

63. The method of claim 61, wherein n is 8.

64. The method of claim 61, wherein the moiety of 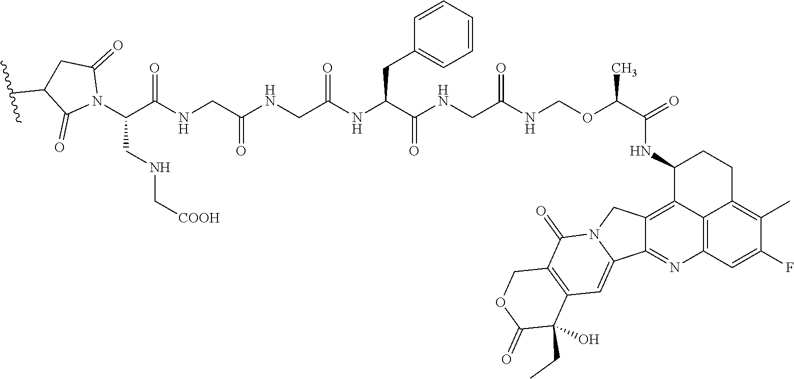 [SMILES: *C1CC(=O)N([C@@H](CNCC(=O)O)C(=O)NCC(=O)NCC(=O)N[C@@H](Cc2ccccc2)C(=O)NCC(=O)NCO[C@@H](C)C(=O)N[C@H]2CCc3c(C)c(F)cc4nc5c(c2c34)Cn2c-5cc3c(c2=O)COC(=O)[C@]3(O)CC)C1=O]
is covalently attached to one or more sulfur atoms of one or more cysteine residues of SI-1×6.4, and wherein each instance of 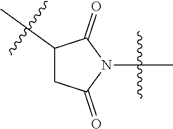 [SMILES: *C1CC(=O)N(*)C1=O]
is independently and optionally replaced by

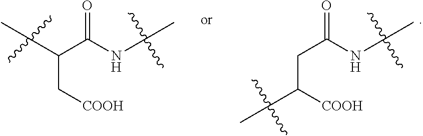 [SMILES: *NC(=O)C(*)CC(=O)O ; *NC(=O)CC(*)C(=O)O]

65. The method of claim 64, wherein the one or more cysteine residues are derived from the opening of one or more interchain disulfide bonds of SI-1×6.4.

66. The method of claim 52, wherein the antibody-drug conjugate is a pharmaceutically acceptable salt of: 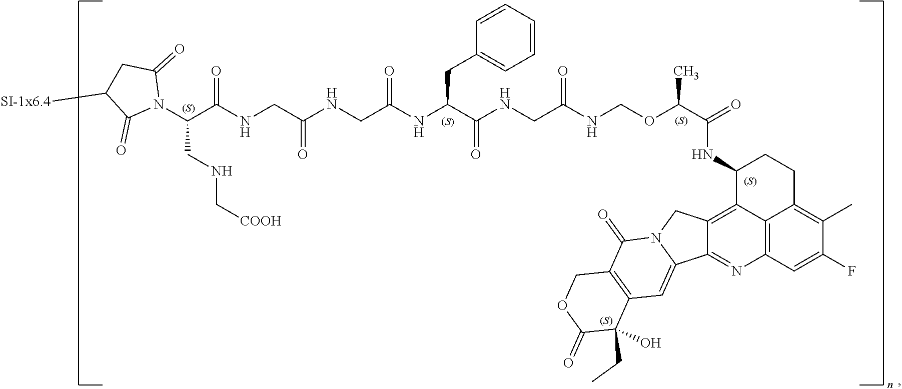
wherein each instance of 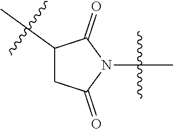 [SMILES: *C1CC(=O)N(*)C1=O]
is independently and optionally replaced by 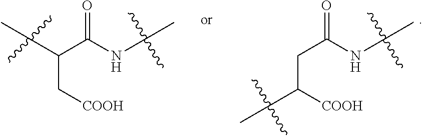 [SMILES: *NC(=O)C(*)CC(=O)O ; *NC(=O)CC(*)C(=O)O]
wherein SI-1×6.4 is a bispecific antibody comprising two IgG1 heavy chains, two κ light chains, and two scFvs, wherein the first IgG1 heavy chain and the first scFv form a construct comprising the amino acid sequence of SEQ ID NO: 4; the second IgG1 heavy chain and the second scFv form a construct comprising the amino acid sequence of SEQ ID NO: 4; the first κ light chain comprises the amino acid sequence of SEQ ID NO: 2; and the second κ light chain comprises the amino acid sequence of SEQ ID NO: 2, and
wherein n is 5, 6, 7, or 8.

67. The method of claim 52, wherein the antibody-drug conjugate is administered in a pharmaceutical composition comprising a multiplicity of the antibody-drug conjugates and a pharmaceutically acceptable carrier, wherein the average drug-to-antibody ratio (“DAR”) of the pharmaceutical composition is from 6 to 8.

68. The method of claim 52, wherein the cancer is adenocarcinoma, ovarian cancer, cervical cancer, uterine cancer, prostate cancer, renal cancer, urethral cancer, bladder cancer, liver cancer, gastric cancer, endometrial cancer, salivary gland cancer, esophageal cancer, lung cancer, non-small cell lung cancer, breast cancer, colon cancer, rectal cancer, colorectal cancer, bone cancer, skin cancer, thyroid cancer, pancreatic cancer, melanoma, glioma, neuroblastoma, glioblastoma multiforme, sarcoma, or lymphoma and leukemia.

69. The method of claim 68, wherein the cancer is non-small cell lung cancer.
